(19) United States
(12) Patent Application Publication
(10) Pub. No.: US 2023/0210453 A1
(43) Pub. Date: Jul. 6, 2023

(54) ORTHODONTIC APPLIANCE COMPLIANCE MONITORING SYSTEMS, DEVICES, AND METHODS

(71) Applicant: Biolux Research Holdings Inc, Anieres (CH)

(72) Inventors: Peter Robert Brawn, Lehi, UT (US); Paul MATHEWS, Langley, WA (US); Robert MARSANYI, Clinton, WA (US); Ben MATTISON, Vancouver (CA)

(21) Appl. No.: 17/309,459

(22) PCT Filed: Nov. 26, 2019
(86) PCT No.: PCT/US2019/063197

§ 371 Date: Mar. 6, 2023

Related U.S. Application Data
(60) Provisional application No. 62/772,288, filed on Nov. 28, 2018.
(60) Provisional application No. 62/890,361, filed on Aug. 22, 2019.

(51) Int. Cl. A61B 5/00; A61C 19/06; A61N 5/06; A61C 7/08; A61B 5/01

(52) U.S. Cl. CPC A61B 5/4833; A61C 19/06; A61N 5/0603; A61C 7/08; A61B 5/01; A61B 5/0008; A61N 2005/0606; A61N 2005/0651; A61N 2005/0643

(57) ABSTRACT

The present invention relates generally to systems, devices, and methods for tracking compliance of orthodontic appliance usage, and more specifically to systems, devices, and methods for tracking compliance of orthodontic appliance usage using a compliance device and/or a light therapy apparatus. The invention provides compliances device that can be configured to be disposed within a patient's mouth. In some embodiments, the compliance device can comprise a sensor configured to detect an input associated with whether an orthodontic appliance is at least partially disposed within the patient's mouth at a first time and at a second time, with the second time being subsequent to the first time.

### CROSS-REFERENCE TO RELATED APPLICATIONS

This application claims the benefit of U.S. Provisional Application No. 62/772,288, filed Nov. 28, 2018, and U.S. Provisional Application No. 62/890,361, filed Aug. 22, 2019, each of which is incorporated by reference herein in its entirety.

### FIELD OF THE INVENTION

The disclosure relates generally to systems, devices, and methods for tracking compliance of orthodontic appliance usage, and more specifically to systems, devices, and methods for tracking compliance of orthodontic appliance usage using a compliance device and/or a light therapy apparatus.

### BACKGROUND OF THE INVENTION

Orthodontics involves the movement of teeth through bone. By applying pressure to a tooth, bone can be broken down at a leading edge of the tooth to facilitate tooth movement. New bone is then created at a trailing edge of the tooth. Bone is resorbed in (e.g., broken down) in areas of pressure between a tooth root and periodontium, and bone is deposited (created) in areas of tension between a tooth root and periodontium. Pressure can cause resorption and tension can cause deposition regardless of where they occur along a tooth root surface. Movement of teeth through bone is slow based on the speed of the remodeling process while teeth are undergoing orthodontic treatment, thereby necessitating treatments of long duration in order to achieve the desired tooth position. Tooth movement in adults is slower than tooth movement in adolescents. Long-term orthodontic treatment can have an increased risk of root resorption, gingival inflammation and dental caries. Moreover, movement of teeth through bone can be uneven, as teeth might “tip” due to the force applied, i.e., the crown of the tooth can move in the desired direction more quickly than the root of the tooth, resulting in tipping of the tooth. When teeth move “bodily” through the bone, i.e., in a more or less perpendicular orientation relative to the bone, the teeth move without tipping or with only a low degree of tipping.

Methods for increasing the rate of tooth movement without damage to the tooth and periodontium have been sought. For example, acceleration of tooth movement can be achieved by the local injection of prostaglandin, the active form of vitamin D3, and osteocalcin around the alveolar socket. These substances might increase the rate of tooth movement, but might also cause side effects such as local pain and discomfort for a patient during the process of injection. In a recent study, more than 65% of the subjects in North America have been shown to be deficient vitamin D serum levels. In these vitamin D-deficient subjects, bone metabolism and remodeling can be adversely affected.

A significant amount of malocclusion in dental patients is caused by lack of sufficient horizontal or vertical growth of the mandibular bone. This can result in the lower teeth being positioned too far in a posterior direction, leading to an increased overjet and retrusive chin. In some situations, the mandibular bone can be too far forward or backward, and it can be desirable to move or remodel the mandibular bone. In other situations, it can be desirable for the maxillary bone to be remodeled.

Typical treatments involve surgical advancement or dental compensatory orthodontic treatment. Some more recent therapy involves the functional repositioning of the mandibular bone forward using an intra-oral orthodontic appliance. This repositioning of the mandibular bone creates remodeling of the temporomandibular joint (TMJ) and also some tooth movement as compensation to the forces. The problem, however, with this approach is that it can take up to 12 months to correct the mandibular position. Additionally, such approaches appear to have much less effectiveness in adults or non-growing adolescents.

### SUMMARY OF THE INVENTION

Described herein are compliance devices, systems, and methods for measuring compliance, and methods for making the same. The invention provides compliances device that can be configured to be disposed within a patient's mouth. In some embodiments, the compliance device can comprise a sensor configured to detect an input associated with whether an orthodontic appliance is at least partially disposed within the patient's mouth at a first time and at a second time, with the second time being subsequent to the first time. The compliance device can be configured to be coupled to an orthodontic appliance or to a tooth of the patient, and to store data associated with the input detected by the sensor at the first time and the second time. In some embodiments, the compliance device can comprise a transceiver or a light emitter.

In some embodiments, the compliance device can be configured to be disposed within a patient's mouth and can comprise a sensor, a power source, and a transceiver. The sensor can be configured to detect whether an orthodontic appliance is disposed within the patient's mouth. The power source can be configured to be recharged based on light received from a light therapy apparatus when disposed within the patient's mouth. The transceiver can be configured to wirelessly communicate to the light therapy apparatus a signal that includes data associated with detection by the sensor.

The invention further provides orthodontic appliances that can comprise a compliance device. In some embodiments, the orthodontic appliance can comprise a compliance device and can be configured to fit within a patient's mouth. The compliance device can be configured to detect a temperature of the patient's mouth at a first time and a second time, with the second time being subsequent the first time. The compliance device can be configured to optically and wirelessly communicate to a light therapy apparatus when disposed within the patient's mouth a signal associated with the temperature of the patient's mouth at the first time and the temperature within the patient's mouth at the second time.

The invention further provides methods. In some embodiments, the method can comprise allowing a light therapy apparatus to receive from a compliance device a signal. In The light therapy apparatus can comprise a mouthpiece and a plurality of light emitters and the mouthpiece and the plurality of light emitters can be configured to be disposed within a patient's mouth. The compliance device can be coupled to a removable orthodontic appliance that can be configured to be disposed within the patient's mouth, or to a tooth of the patient. The compliance device can be configured to detect a temperature of the patient's mouth at a first time and a second time, with the second time being subsequent the first time. The signal can comprise data associated with the temperature of the patient's mouth at the first time and the second time.

### INCORPORATION BY REFERENCE

Each publication, patent, and patent application referenced in this specification is herein incorporated by reference in its entirety.

### BRIEF DESCRIPTION OF THE DRAWINGS

Reference is now made to the following detailed description and the accompanying drawings of which:

FIG. 1 is an isometric view of an embodiment of a light-therapy apparatus useful for providing light therapy to specified regions of a patient's maxillary or mandibular alveolar bone.

FIG. 2 is a front view of the embodiment shown in FIG. 1.

FIG. 3 is a top view of the embodiment shown in FIG. 1.

FIG. 4 is a right isometric view of the embodiment shown in FIG. 1.

FIG. 5 is a schematic cross-sectional view through a portion of a light source having a light emitter and a reflector.

FIG. 6 is a top view of a programmable controller for use with a light-therapy apparatus.

FIG. 7A is a partial cross-sectional view of a support arm of an embodiment of a light-therapy apparatus showing the engagement between a track engaging ridge on a light source and a track formed in the support arm.

FIG. 7B is a partial cross-sectional view of a support arm of an embodiment of a light-therapy apparatus showing the engagement between a track engaging ridge on a heat sink and a track formed in the support arm.

FIG. 8A shows a first view of a light-therapy apparatus in accordance with an embodiment of the invention.

FIG. 8B is a rear view of the light-therapy apparatus of FIG. 8A.

FIG. 8C is a side view of the light-therapy apparatus of FIG. 8A.

FIG. 8D is a front view of the light-therapy apparatus of FIG. 8A.

FIG. 9A is a perspective view of a light-therapy apparatus having an intra-oral tray, an extra-oral bridge, and left and right side extra-oral LED arrays.

FIGS. 9B, 9C and 9D are respectively a cross-section, a front side elevation and a rear elevation of a light source having a cooling fan, a heat sink and two arrays of light emitters.

FIG. 10 is a side view of a portion of the light-therapy apparatus of FIG. 9A with the end of the extra-oral bridge attached to the extra-oral LED array and with the extra-oral LED array in a first orientation and a second orientation (shown in broken lines) with respect to the extra-oral bridge.

FIG. 11 is a view from the front-left side of the light-therapy apparatus of FIG. 9A with a portion of the extra-oral LED array detached.

FIG. 12 is a view from the rear right side of a portion of the light-therapy apparatus of FIG. 9A.

FIG. 13 is a view from the left rear side of a portion of the light-therapy apparatus FIG. 9A with the intra-oral tray detached.

FIG. 14 is a perspective view of a light-therapy apparatus according to an embodiment in which an LED array is supported by a head-set.

FIG. 15 is a side view of the light-therapy apparatus of FIG. 14.

FIG. 16 is a perspective view of a light-therapy apparatus according to an embodiment in which an LED array is supported by a head-set.

FIG. 17 is a front view of at least one LED array, and a connector detached from the head-set.

FIG. 18 is a front view of an external light-therapy apparatus having two LED arrays, a hinge-like member, and an attaching means.

FIG. 19 is a cross-sectional view of an LED array mounted onto a substrate.

FIG. 20 is a cross-sectional view of an LED array detached from the substrate.

FIG. 21A is a perspective view of a light-therapy apparatus in accordance with another embodiment of the invention.

FIG. 21B shows a close up of an example of how a light source is supported in the light-therapy apparatus.

FIG. 22A shows an obverse view of a controller in accordance with another embodiment of the invention.

FIG. 22B shows a reverse view of the controller.

FIGS. 23 and 24 are perspective views of intra-oral light-therapy apparatuses according to an embodiment of the invention.

FIG. 25A is a sectional view of the apparatus of FIG. 1, taken along line X-X.

FIG. 25B is a side view of a portion of an intra-oral light-therapy apparatus according to an embodiment of the invention.

FIGS. 26-30 are schematic diagrams of intra-oral light-therapy apparatuses according to an embodiment of the invention.

FIG. 31 is a schematic diagram of an intra-oral light-therapy apparatus according to an embodiment of the invention.

FIG. 32 is a sectional view of a portion of the apparatus of FIG. 31.

FIG. 33 is a top perspective view of a portion of the apparatus of FIG. 31.

FIG. 34 is a sectional view of a portion of the apparatus of FIG. 31.

FIG. 35 is a schematic diagram of an intra-oral light-therapy apparatus according to an embodiment of the invention.

FIG. 36 is a side view of the apparatus of FIG. 35.

FIG. 37 is a side view of the apparatus of FIG. 35 and a charging station.

FIGS. 38 and 39 are schematic diagrams of intra-oral light-therapy apparatuses according to an embodiment of the invention.

FIG. 40A is a top view of a portion of an intra-oral light-therapy apparatus according to an embodiment of the invention.

FIG. 40B is a front view of a portion of an intra-oral light-therapy apparatus according to an embodiment of the invention.

FIG. 41 is a top view of a portion of an intra-oral light-therapy apparatus according to an embodiment of the invention.

FIG. 42 is a perspective view of an intra-oral light-therapy apparatus according to an embodiment of the invention.

FIG. 43 is a sectional view of the apparatus of FIG. 42 taken along line A-A.

FIG. 44 is a bottom view of the apparatus of FIG. 42.

FIG. 45 is a side view of an intra-oral light-therapy apparatus according to an embodiment of the invention in use within an oral cavity.

FIGS. 46 and 47 are side and front views, respectively, of portions of intra-oral light-therapy apparatuses according to embodiments of the invention.

FIG. 48 is perspective view of an intra-oral light-therapy apparatus according to an embodiment of the invention.

FIGS. 49-51 are top views of intra-oral light-therapy apparatuses according to embodiments of the invention.

FIGS. 52-55 are perspective views of an intra-oral light-therapy apparatus according to an embodiment of the invention.

FIGS. 56 and 57 are top and rear views, respectively, of the apparatus of FIGS. 52-55 in a powered (i.e., “on”) operational state.

FIG. 58 is a perspective view of the apparatus of FIGS. 56 and 57 coupled to an electronic device.

FIG. 59 is an image of the apparatus of FIGS. 52-58 disposed in the oral cavity of and in use by a patient.

FIG. 60 is an image of an intra-oral light-therapy apparatus according to an embodiment of the invention disposed in the oral cavity of and in use by a patient.

FIG. 61 is an image of an upper arch of a patient prior to light therapy treatment using the intra-oral light-therapy apparatus of FIG. 60.

FIG. 62 is an image of the patient's upper arch of FIG. 61 after light therapy treatment using the intra-oral light-therapy apparatus of FIG. 60.

FIG. 63 is an image of the upper arch of a patient prior to light therapy treatment using the intra-oral light-therapy apparatus of FIG. 60.

FIG. 64 is an image of the patient's upper arch of FIG. 63 after light therapy treatment using the intra-oral light-therapy apparatus of FIG. 60.

FIG. 65 is a rear view of an intra-oral apparatus according to an embodiment of the invention.

FIG. 66 is a front view of the intra-oral apparatus of FIG. 65.

FIGS. 67 and 68 are side and top views of an intra-oral apparatus according to an embodiment of the invention.

FIG. 69 is a perspective view of a portion of the intra-oral apparatus of FIG. 67.

FIGS. 70A and 70B are bottom-rear and top-rear perspective views of a portion of the intra-oral apparatus of FIG. 67.

FIGS. 71 and 72 are perspective and front views of the intra-oral apparatus of FIG. 67 and an external station according to an embodiment of the invention.

FIG. 73 is a front view of the external station of FIG. 71.

FIG. 74 is a schematic illustration of a portion of the intra-oral apparatus of FIG. 67.

FIG. 75 is a top view of an intra-oral apparatus according to an embodiment of the invention.

FIG. 76 is a side view of a portion of an intra-oral apparatus according to an embodiment of the invention.

FIGS. 77 and 78 are end and perspective views of the intra-oral apparatus of FIG. 76.

FIG. 79 is a schematic illustration of a system including the intra-oral apparatus of FIG. 76 according to an embodiment of the invention.

FIG. 80 is a schematic illustration of an intra-oral apparatus according to an embodiment of the invention.

FIG. 81 is a graphical illustration of individual rates of maxillary tooth movement for study group participants.

FIG. 82 is a graphical illustration of individual rates of mandibular tooth movement for study group participants.

FIG. 83 is a perspective view of a comparative extra-oral light therapy apparatus.

FIGS. 84 and 85 are perspective views of a light therapy apparatus according to an embodiment of the invention.

FIG. 86A is a right side view of the light therapy apparatus of FIG. 84. The left side view of the light therapy apparatus of FIG. 84 is a mirror image to the right side view.

FIG. 86B is a cross-sectional view of the apparatus of FIG. 86A, taken along line B-B in FIG. 86A.

FIG. 87 is a rear perspective view of the light therapy apparatus of FIG. 84.

FIG. 88 is a perspective view of the light therapy apparatus of FIG. 84 in an inverted position.

FIG. 89 is a perspective view of a portion of the light therapy apparatus of FIG. 84.

FIG. 90 is a front view of the portion of the light therapy apparatus of FIG. 89.

FIG. 91 is a top view of the portion of the light therapy apparatus of FIG. 89.

FIG. 92 is a bottom view of the portion of the light therapy apparatus of FIG. 89.

FIG. 93 is a perspective view of a portion of the light therapy apparatus of FIG. 84.

FIG. 94 is a rear view of the light therapy apparatus of FIG. 84.

FIG. 95 is a schematic view of a portion of the light therapy apparatus of FIG. 84.

FIGS. 96A-96F are electrical schematics of portions of the light therapy apparatus of FIG. 84. FIG. 96A illustrates an accelerometer of the light therapy apparatus of FIG. 84 according to embodiments. FIG. 96B illustrates a microcontroller of the light therapy apparatus of FIG. 84 according to embodiments. FIG. 96C illustrates a switch of the light therapy apparatus of FIG. 84 according to embodiments. FIG. 96D illustrates an induction coil for wireless charging of the light therapy apparatus of FIG. 84 according to embodiments. FIG. 96E illustrates a temperature sensor of the light therapy apparatus of FIG. 84 according to embodiments. FIG. 96F illustrates a capacitive sensor of the light therapy apparatus of FIG. 84 according to embodiments.

FIG. 97 is a perspective view of a portion of the light therapy apparatus of FIG. 84.

FIGS. 98-108 are logic flow charts representing code configured to be executed by the light therapy apparatus of FIG. 84, according to various embodiments.

FIG. 109 is a perspective view of an external station according to an embodiment, within which the light therapy apparatus of FIG. 84 can be disposed.

FIG. 110 is a rear view of the external station of FIG. 109.

FIG. 111 is a top view of a bottom portion of the external station of FIG. 109.

FIG. 112 is a perspective view of the bottom portion of the external station of FIG. 109.

FIG. 113 is a schematic illustration of electronic components of the external station of FIG. 109.

FIG. 114 is a side view of a barrier implant implanted at an extraction site according to an embodiment of the invention.

FIGS. 115-125 are images of sample display screens of an external electronic device according to an embodiment of the invention.

FIGS. 126-127 are rear perspective and rear views of a light therapy apparatus according to an embodiment of the invention.

FIG. 128 is a top view of a light therapy apparatus according to an embodiment of the invention.

FIG. 129 is a perspective view of the light therapy apparatus of FIG. 128.

FIG. 130 is a top perspective view of the light therapy apparatus of FIG. 128.

FIG. 131 is a rear view of the light therapy apparatus of FIG. 128.

FIG. 132 is a perspective view of the light therapy apparatus of FIG. 128.

FIG. 133 is a front view of a portion of a light therapy apparatus according to an embodiment of the invention.

FIGS. 134-135 are rear perspective and rear views of a light therapy apparatus according to one or more embodiments.

FIG. 136 is a top view of a light therapy apparatus according to one or more embodiments.

FIG. 137 is a perspective view of the light therapy apparatus of FIG. 136.

FIG. 138 is a top perspective view of the light therapy apparatus of FIG. 136.

FIG. 139 is a rear view of the light therapy apparatus of FIG. 136.

FIG. 140 is a perspective view of the light therapy apparatus of FIG. 136.

FIGS. 141A-141F are illustrative embodiments of a light therapy apparatus.

FIG. 142 is a flow chart of a method according to an embodiment of the invention.

FIG. 143 is an image of a sample display screen of an external electronic device according to an embodiment of the invention.

FIGS. 144A, 144B, 145A, and 145B are tables of individual patient data during a space closure phase of orthodontic treatment for study group participants.

FIGS. 146-148 are bar graphs showing an average number of days per aligner for individual patients in the study described in Example 12.

FIG. 149 is a schematic of one or more embodiments of the light therapy apparatus disclosed herein.

FIG. 150 is a schematic of the crossover trial design and orthodontic procedures described in Example 13.

FIG. 151 is a consort chart of the patients screened for the study set forth in Example 13.

FIGS. 152A and 152B show two cases treated with orthodontic brackets and wires, and FIGS. 152C and 152D show two cases treated with intra-oral light therapy. FIG. 152A—Baseline (Day 0); LII is 8.80 mm. FIG. 152B—Day 131; LII is 0.00 mm. FIG. 152C—Baseline (Day 0); LII is 9.07 mm. FIG. 152D—Day 50; LII is 0.00 mm.

FIG. 153 illustrates magnitude of tooth movement in response to Vitamin D supplementation and intra-oral light therapy in rats.

FIG. 154 illustrates MicroCT analyses of bone specimens in response to TM with or without Vitamin D supplementation and intra-oral light therapy in rats.

FIG. 155 illustrates osteocalcin expression around first molars in the TM+PBM+VitD group (group 5) in response to intra-oral light therapy, vitamin D administration, and conventional tooth movement in rats.

FIG. 156A is a perspective view of a light therapy apparatus according to an embodiment of the invention.

FIG. 156B is a top view of the light therapy apparatus of FIG. 156A.

FIG. 156C is a back/posterior view of the light therapy apparatus of FIG. 156A.

FIG. 156D is a side view of the light therapy apparatus of FIG. 156A.

FIG. 157A is a perspective view of a portion of a light therapy apparatus according to an embodiment of the invention.

FIG. 157B is an exploded view of the portion of the light therapy apparatus of FIG. 157A.

FIG. 157C is a top view of a light therapy apparatus according to an embodiment.

FIG. 157D is a perspective view of the light therapy apparatus of FIG. 157C.

FIG. 157E is a first side view of the light therapy apparatus of FIG. 157C.

FIG. 157F is a rear view of the light therapy apparatus of FIG. 157C.

FIG. 157G is a second side view of the light therapy apparatus of FIG. 157C.

FIGS. 157H-I show example views of a patient's mouth (upper dental arch and lower arch), with an overlaid outline of a light therapy apparatus disposed therein.

FIGS. 158A-158C are schematic illustrations of embodiments of compliance monitoring systems.

FIG. 159 is a schematic illustration of a portion of the compliance monitoring system of FIG. 1, according to an embodiment.

FIG. 160 is a side view of a compliance device and an orthodontic appliance, according to an embodiments.

FIG. 161A is a perspective schematic illustration of an embodiment of a compliance device showing some dimensions of the device, according to some embodiments.

FIG. 161B illustrates exemplary front, top, and side views of an illustrative compliance device, and an exemplary actual size of an illustrative compliance device, according to some embodiments. FIG. 161C depicts an illustrative compliance device coupled to a user's tooth.

FIG. 162A illustrates placement of an orthodontic appliance with a compliance device on teeth of a user. FIG. 162B illustrates optical transmission between compliance monitoring of the orthodontic appliance-compliance device setup of FIG. 162A using and a light therapy apparatus, according to some an embodiments, with the light therapy apparatus being spaced apart from the orthodontic appliance of FIG. 162A in the direction of arrow A-A for illustration purposes.

FIG. 163 illustrates a method for tracking compliance of orthodontic appliance use, according to some embodiments.

FIGS. 164A and 164B depict an illustrative probe useful with a compliance device, according to some embodiments.

FIG. 165 depicts a cross-sectional view of an illustrative compliance monitoring device, according to some embodiments.

### DETAILED DESCRIPTION OF THE INVENTION

Throughout the following description specific details are set forth in order to provide a more thorough understanding to persons skilled in the art. However, well known elements might not have been shown or described in detail to avoid unnecessarily obscuring the disclosure. Accordingly, the description and drawings are to be regarded in an illustrative, rather than a restrictive, sense.

The terms “about” and “approximately” as used herein in connection with a referenced numeric indication means the referenced numeric indication plus or minus up to 10% of that referenced numeric indication. For example, the language “about 50” units or “approximately 50” units means from 45 units to 55 units.

The verb “surround” as used herein means to be spaced less than about one (1) centimeter of a target object. For example, oral tissue that surrounds a tooth is spaced less than about 1 cm from the tooth. In one or more embodiments, the apparatuses, systems and methods disclosed herein are useful for preventing or minimizing inflammation that is less than about 1 cm from a tooth.

The term “patient” as used herein refers to any living subject that can receive medical, including orthodontic, treatment. A patient can be, for example, a mammal such as a human. The patient can be an adult patient or a child patient. In one or more embodiments, the patient can be a living subject that receives light treatment, e.g., light administered to the patient intra-orally using an intra-oral apparatus described herein.

In some embodiments, the patient is an adolescent or a pre-adolescent. In some such embodiments, the adolescent is undergoing a growth spurt. In some embodiments, the patient is a living subject that receives light treatment, e.g., light administered to the patient extra-orally or intra-orally. In some such embodiments, the patient wears an orthodontic appliance (e.g., a functional appliance or another appliance). The orthodontic appliance can be worn or otherwise donned during the time the patient receives light treatment (e.g., during bone remodeling treatment). In other embodiments, however, the patient had worn, or previously wore, an orthodontic appliance prior to being administered with an effective amount of light transdermally or nontransdermally to a region of the patient's oral or maxillofacial bone, muscle, or soft tissue, or to one or more teeth. In yet other embodiments, the patient wears an orthodontic appliance subsequent to being administered with an effective amount of light transdermally or nontransdermally to a region of the patient's oral or maxillofacial bone, muscle, or soft tissue, or to one or more teeth. In some embodiments, the patient's oral or maxillofacial bone, muscle, or soft tissue comprises the patient's maxillary or mandibular alveolar bone.

The term “user” as used herein includes, but is not limited to, a patient, an orthodontist, another orthodontic-care provider, an orthodontic device or appliance manufacturer, an orthodontic device or appliance wholesaler, and an orthodontic device or appliance retailer.

As used herein, “alveolar mucosa” refers to oral mucosa that is immediately apical to the mucogingival junction. Alveolar soft tissue (such as alveolar mucosa) is distinguished from gum tissue (or “gingiva”), for example, by the patient's mucogingival junction. The mucogingival junction is a line of demarcation between gum tissue and the alveolar mucosa, and gum tissue is therefore distinct from alveolar mucosa.

The term “root area” as used herein refers to a portion of a patient's anatomy that includes the anatomic length and width of a tooth root, as well as at least a portion of peripheral tissue that facilitates attachment of the tooth to the alveolar bone within which the tooth sits. The peripheral tissue can include the periodontal ligament and the boney socket in which the periodontal ligament is disposed and which surround the tooth. The root area can include tissue extending from the gum line to a depth of about 10 mm to about 22 mm, depending on the type of tooth. The root area can also include an area within a particular distance (e.g., at a distance from about 0.1 cm to about 3 cm) of the root area of each tooth, unless the context clearly indicates otherwise. The dimensions of a root area can vary depending on the particular subject tooth. References to the root area herein can include at least a portion of the root area or the entirety of the root area, unless the context clearly indicates otherwise.

As used herein, the term “flexibility” relates to an object's resistance to deflection, deformation, and/or displacement by an applied force. For example, a mouthpiece or oral structure with greater flexibility is less resistant to deflection, deformation, and/or displacement when exposed to a force than a mouthpiece or oral structure with lower flexibility. A mouthpiece with higher stiffness can be characterized as being more rigid (or less flexible) than a mouthpiece with lower stiffness. In one or more embodiments, the flexibility and/or deformability of an object can be characterized by the object's linear flexibility and/or deformability. Linear flexibility and/or deformability can be characterized in terms of the amount of force applied to the object and the resulting distance through which a first portion of the object deflects, deforms, and/or displaces with respect to a second portion of the object.

Flexibility is an extensive property of the object being described, and thus is dependent upon the material from which the object is formed and particular physical characteristics of the object (e.g., shape and boundary conditions). For example, the flexibility of an object can be increased or decreased by selectively including in the object a material having a desired modulus of elasticity. The modulus of elasticity is an intensive property of the constituent material and describes an object's tendency to elastically (i.e., non-permanently) deform in response to an applied force. A material having a high modulus of elasticity will not deflect as much as a material having a low modulus of elasticity in the presence of an equally applied force. Thus, the flexibility of the object can be increased, for example, by introducing into the object and/or constructing the object of a material having a low modulus of elasticity. In another example, the flexibility of the object can be increased or decreased by changing the flexural modulus of a material of which the object is constructed. Flexural modulus is used to describe the ratio of the applied stress on an object in flexure to the corresponding strain in the outermost portions of the object. The flexural modulus, rather than the modulus of elasticity, is used to characterize particular materials, for example plastics, that do not have material properties that are linear over a range of conditions. An object with a first flexural modulus is less elastic and has a greater strain on the outermost portions of the object than an object with a second flexural modulus lower than the first flexural modulus. Thus, the flexibility of an object can be increased by including in the object a material having a low flexural modulus.

The flexibility of an object can also be increased or decreased by changing a physical characteristic of the object, such as the shape or cross-sectional area of the object. For example, an object having a length and a cross-sectional area can have a greater flexibility than an object having an identical length but a greater cross-sectional area. Thus, the flexibility and/or stiffness of the object can be increased by increasing and/or changing the shape of the cross-sectional area of the object. In one or more embodiments described herein, the flexibility of an apparatus is increased via the inclusion of a notch in a flange of the apparatus.

The term “substantially rigid” as used herein to describe an apparatus, or a component thereof, means that the various dimensional parameters associated with the apparatus or component (e.g., length, depth, thickness, curvature, angle, etc.) remain about the same when the apparatus is manipulated and/or otherwise used as described herein.

The term “transparent” as used herein relates to an object's ability to transmit light therethrough. The transparency of an object is directly related to the absence of (or very low amounts of) scattering of light within the object. An object is said to be “substantially transparent” if the object allows visible light to be transmitted therethrough such that another object can be distinctly seen through the subject object. In another embodiment, an object is said to be “substantially transparent” if the object permits transmission of at least sixty percent of incident light in a visible range through a portion of the object, as measured by any applicable test, such as ASTM D-1746, ASTM D-1003 or the like. In yet other embodiments, an object is said to be “substantially transparent” if the object permits transmission of at least seventy percent of incident light in a visible range through a portion of the object. In yet other embodiments, an object is said to be “substantially transparent” if the object permits transmission of at least eighty percent of incident light in a visible range through a portion of the object. In yet other embodiments, an object is said to be “substantially transparent” if the object permits transmission of at least ninety percent of incident light in a visible range through a portion of the object. In yet other embodiments, an object is said to be “substantially transparent” if the object permits transmission of at least ninety-five percent of incident light in a visible range through a portion of the object. In yet other embodiments, an object is said to be “substantially transparent” if the object permits transmission of at least ninety-nine percent of incident light in a visible range through a portion of the object.

Systems, devices, and methods for tracking compliance of orthodontic appliance usage are provided herein. A compliance device according to an embodiment that is configured to be disposed within a patient's mouth comprises a sensor configured to detect at a first time and at a second time, subsequent to the first time, an input associated with whether an orthodontic appliance is at least partially disposed within the patient's mouth. The compliance device is configured to be coupled to the orthodontic appliance or to a tooth of the patient. The compliance device is configured to store data associated with input detected by the sensor at the first time and the second time. The compliance device comprises a transceiver or a light emitter. The compliance device can comprise a casing configured to be bonded to the tooth. The input can be a detection of a capacitance change.

A system according to an embodiment comprises a compliance device as described herein and a light therapy apparatus. The light therapy apparatus comprises a mouthpiece configured to be disposed within the patient's mouth proximate to or in contact with the compliance device. The light therapy apparatus comprises a transceiver or a light emitter, which is configured to receive a wireless signal from the compliance device. The signal comprises data associated with input detected by the sensor at the first time and the second time. In some embodiments, the system further comprises an orthodontic appliance configured to be at least partially disposed within the patient's mouth, and the compliance device is coupled to the orthodontic appliance. In some embodiments, the signal is a first signal, the input comprises an indication of a capacitance change, a pressure, magnetism, or a physiological parameter, and the light therapy apparatus is configured to wirelessly transmit a second signal comprising data associated with the input to an external electronic device. In some embodiments, the sensor is configured to detect a capacitance change produced by the orthodontic appliance when the orthodontic appliance is disposed within a predetermined distance from the compliance device. In some embodiments, the compliance device includes a magnet configured to be bonded to the tooth, and the sensor is configured to detect magnetism produced by the appliance when the appliance is disposed within a predetermined distance of the magnet. In some embodiments, the orthodontic appliance is an aligner, and at least a portion of the aligner is optically transparent or optically translucent. In some embodiments, the orthodontic appliance is a removable aligner. In some embodiments, the orthodontic appliance is a first orthodontic appliance of a plurality of orthodontic appliances, the compliance device is a first compliance device, and the system further comprises a second compliance device coupled to a second orthodontic appliance of the plurality of orthodontic appliances, wherein the second compliance device (i) does not comprise a sensor configured to detect the input or (ii) is configured to not detect the input at either the first time or the second time. In some embodiments, the input is the physiological parameter, and the second compliance device (i) does not comprise a sensor configured to detect the physiological parameter and (ii) is configured to not detect the physiological parameter at either the first time or the second time. In some embodiments, the compliance device comprises a light emitter configured to optically communicate the signal to the light therapy apparatus. In some embodiments, the light emitter is a first light emitter, the light therapy apparatus comprises a second light emitter that is different than the first light emitter, and the second light emitter is configured to optically receive, from the first light emitter, the signal from the compliance device. In some embodiments, the light therapy apparatus is configured to provide power to the compliance device via light emitted by the second light emitter. In some embodiments, the first emitter and the second emitter are light emitting diodes (LEDs). In some embodiments, the compliance device further comprises a rechargeable power source, and the compliance device is configured to use light energy received from the light therapy apparatus to charge the power source.

In some embodiments, a compliance device is configured to be disposed within a patient's mouth and comprises a sensor. The sensor is configured to detect whether an orthodontic appliance is disposed within the patient's mouth. The compliance device comprises a power source that is configured to be recharged based on light received from a light therapy apparatus when disposed within the patient's mouth. The compliance device further comprises a transceiver that is configured to wirelessly communicate to the light therapy apparatus a signal that includes data associated with detection by the sensor. In some embodiments, the compliance device is configured to be coupled to the orthodontic appliance or to a tooth of the patient. In some embodiments, a system comprises the compliance device as described herein and a light therapy apparatus.

An orthodontic appliance, according to an embodiment, that is configured to be disposed within a patient's mouth comprises a compliance device. The compliance device is configured to detect a temperature of the patient's mouth at a first time and a second time, the second time is subsequent the first time. The compliance device is configured to optically and wirelessly communicate to a light therapy apparatus when disposed within the patient's mouth a signal associated with the temperature of the patient's mouth at the first time and the temperature within the patient's mouth at the second time. In some embodiments, the compliance device is embedded in the orthodontic appliance.

A method, according to an embodiment, comprises allowing a light therapy apparatus to receive from a compliance device a signal. The light therapy apparatus comprises a mouthpiece and a plurality of light emitters, and the mouthpiece and the plurality of light emitters are configured to be disposed within a patient's mouth. The compliance device is coupled to (i) a removable orthodontic appliance that is configured to be disposed within the patient's mouth or (ii) a tooth of the patient, the compliance device being configured to detect a temperature of the patient's mouth at a first time and a second time, the second time being subsequent the first time. The signal comprises data associated with the temperature of the patient's mouth at the first time and the second time. In some embodiments, the signal is a first signal, and the method further comprises allowing the light therapy apparatus to wirelessly transmit to an external electronic device a second signal that comprises data associated with the temperature of the patient's mouth at the first time and the second time. In some embodiments, the orthodontic appliance is a first orthodontic appliance of a plurality of orthodontic appliances, the compliance device is a first compliance device coupled to the first orthodontic appliance, and the method further comprises (1) allowing the light therapy apparatus to optically receive from a second compliance device an indication of additional temperature information. The second compliance device is coupled to a second orthodontic appliance of the plurality of orthodontic appliances, and (2) generating compliance information based on the data from the first signal and second signal. In some embodiments, the compliance device comprises a first light emitter that is configured to optically communicate the signal to the light therapy apparatus and the light therapy apparatus comprises a second light emitter that is configured to optically receive the signal. In some embodiments, the compliance device comprises a rechargeable power source that is operably coupled to the first light emitter, and the method further comprises irradiating light to the first light emitter from that second light emitter such that the compliance device converts the light to energy capable of charging the rechargeable power source. In some embodiments, the removable orthodontic appliance is an optically transparent aligner.

In some embodiments, a method for regulating bone remodeling comprises disposing an orthodontic appliance within an oral cavity of the patient such that the orthodontic appliance is coupled to one or more teeth of the patient in need of bone remodeling. The method also comprises administering to the patient an effective amount of (a) light from one or more light emitters of a light-therapy apparatus and (b) vitamin D. In some embodiments, the orthodontic appliance is removably coupled to the one or more teeth of the patient. In some embodiments, the orthodontic appliance is fixedly coupled to the one or more teeth of the patient. In some embodiments, regulating bone remodeling is increasing movement of one or more teeth toward alignment.

In some embodiments, the light-therapy apparatus is an extra-oral light-therapy apparatus. In some embodiments, the extra-oral light therapy apparatus is removably coupled to the face of the patient. In some embodiments, the extra-oral light therapy apparatus comprises the one or more light emitters.

In some embodiments, the light therapy apparatus is an intra-oral light therapy apparatus. In some embodiments, the intra-oral light-therapy apparatus comprises a mouthpiece that comprises one or more light emitters. In some embodiments, the intra-oral light therapy apparatus comprises a mouthpiece configured to fit within a patient's mouth. The intra-oral therapy apparatus also comprises a mouthpiece including a bite tray and a flange coupled to the bite tray, and the one or more light emitters is disposed within the flange. In some embodiments, the method further comprises disposing the mouthpiece into the patient's mouth.

In some embodiments, the vitamin D is administered orally. In some embodiments, the vitamin D is in the form of calcitrol.

In some embodiments, the orthodontic appliance comprises an aligner configured for one or both of (a) inducing tooth movement of one or more teeth toward alignment and (b) accelerating tooth movement of the one or more teeth toward alignment.

As will be described in more detail herein, in some embodiments, the patient receives vitamin D treatment in addition to the light treatment. The vitamin D can be administered to the patient prior to, concurrently with, or subsequent to the patient receiving light treatment. In some embodiments, the patient can wear an orthodontic appliance prior to, concurrently with, or subsequent to receiving vitamin D treatment similar to the manner in which the orthodontic appliance was worn prior to, concurrently with, or subsequent to the light treatment. In some embodiments, the patient is not administered with vitamin D, but receives light treatment. In other embodiments, the patient does not receive light treatment, but is administered with vitamin D.

In accordance with some aspects of the invention, methods are provided for regulating bone remodeling. Bone remodeling is one or both of deposition and resorption of bone. In some instances, bone remodeling can include a change in the bone's geometry. The bone can be a patient's skull, spine, pelvis or femur, or one or more teeth. Bone can also be from the patient's oral or maxillofacial region, which includes the maxillary bone, the mandibular bone, the temporal bone, and the like.

Apparatuses and Methods for Extra-Oral Light Therapy

In one embodiment, a method for regulating bone remodeling comprises administering an effective amount of light to oral or maxillofacial bone, muscle, or soft tissue, or to one or more teeth of a patient (also referred to herein as “light treatment”). The oral or maxillofacial bone, muscle, or soft tissue of the patient can include the maxillary bone, maxillary alveolar bone, mandibular bone, mandibular alveolar bone, temporal bone, jaw muscle, jaw soft tissue, or one or more teeth of the patient. As such, the effective amount of light can be administered, for example, to a region of the patient's maxillary bone, mandibular bone, or temporal bone. As will be described in more detail herein, the light can be administered transdermally from an extra-oral light source or nontransdermally from an extra-oral or intra-oral light source.

The method for regulating bone remodeling can also comprise allowing a force to be exerted on the oral or maxillofacial bone, muscle, or soft tissue, or one or more teeth of a patient in need thereof. The force can be exerted before, during, or after the light is administered. Stated another way, the light can be administered before, during, or after the force is exerted. The force can be, for example, a heavy force or a force exerted by an orthodontic appliance, such as a functional appliance or another appliance. In some embodiments, however, the method does not comprise allowing a force to be exerted.

The method for regulating bone remodeling can further comprise administering an effective amount of vitamin D to a patient in need thereof. In other words, the patient is administered both an effective amount of vitamin D and an effective amount of light. In some embodiments, however, the effective amount of vitamin D is administered to the patient in lieu of administering the effective amount of light. As will be described in more detail herein, the vitamin D can be administered before, during, or after the force is exerted and/or before, during, or after the light is administered. In some embodiments, however, the method does not comprise allowing a force to be exerted.

In some embodiments, methods for regulating bone remodeling can also include non-orthodontic embodiments. In some embodiments, methods for regulating bone remodeling can include implant placement, grafting, other bony surgeries, orthopedic surgeries, or spinal surgeries. In some such embodiments, an effective amount of light is administered to the patient. The effective amount of light can be administered to the region of the patient's body where the bone remodeling occurs without application of the present methods. Alternatively, the effective amount of light can be administered to a region of the patient's body where the bone remodeling does not occur without application of the present methods. The effective amount of light can be administered locally to a region of the patient's body. Alternatively, the effective amount of light can be administered systemically. Without being bound by theory, light administered to one region of the body can produce bioactive molecules, such as nitric oxide (NO), which can circulate through the bloodstream and throughout the entire body. In this manner, the light can affect regions of the body that are not directly irradiated with light. More details about nitric oxide are set forth in the following publications, which are each incorporated by reference herein in their entirety: Akin, et al. (2004). “Effects of Nitric Oxide in orthodontic tooth movement in rats,” Am. J. Orthod. Dentofacial Orthop., 126(5): 608-14; Houreld, et al. (2010). “Irradiation at 830 nm stimulates nitric oxide production and inhibits pro-inflammatory cytokines in diabetic wounded fibroblast cells,” Lasers in Surgery and Medicine, 42: 494-502: Moriyama, et al. (2009), “In vivo effects of low level laser therapy on inducible nitric oxide synthase,” Lasers in Surgery and Medicine, 41: 227-231; Shirazi, et al. (2002), “The Role of Nitric Oxide in Orthodontic Tooth Movement in Rats,” Angle Orthod, 72(3): 211-15: Samoilova et al. (2008), “Role of Nitric Oxide in the Visible Light-Induced Rapid Increase of Human Skin Microcirculation at the Local and Systemic Level: 1. Diabetic Patients,” Photomedicine and Laser Surgery, 26(5): 433-442; and Samoilova et al. (2008), “Role of Nitric Oxide in the Visible Light-Induced Rapid Increase of Human Skin Microcirculation at the Local and Systemic Levels: II. Healthy Volunteers,” Photomedicine and Laser Surgery, 26(5): 443-449.

In embodiments where the methods comprise administering vitamin D, the vitamin D can be administered at the region of the patient's body where the bone remodeling occurs. Alternatively, the vitamin D can be administered to a region of the patient's body where the bone remodeling does not occur. The vitamin D can be administered locally to a region of the patient's body. Alternatively, vitamin D can administered systemically.

In some embodiments, the administration of vitamin D enhances (by increasing the rate of, or accelerating) bone metabolism, particularly in the context of accelerating bone remodeling. In some embodiments, administration of vitamin D increases osteoclastic activity. In some embodiments, administration of vitamin D increases bone resorption, and causes faster tooth movement. Bone is resorbed in the path of tooth movement, enabling the tooth to move. In some embodiments, the administration of vitamin D (with or without the administration of light, with or without the exertion of a force) will increase the density and total volume of bone in typically bony skeletons.

In some embodiments, regulating bone remodeling comprises reducing, minimizing or preventing tooth-root resorption. In some embodiments, a method for reducing, minimizing or preventing tooth-root resorption comprises allowing a force to be exerted on one or more teeth of a patient in need thereof, administering vitamin D to the patient and administering an effective amount of light to oral or maxillofacial bone, muscle, or soft tissue, or one or more teeth of the patient, wherein the light is administered before, during, or after the force is exerted. In some embodiments, regulating bone remodeling comprises reducing bone resorption or inflammatory dentin or cementum resorption of the tooth root or periodontium. In some embodiments, methods for reducing bone resorption or inflammatory dentin or cementum resorption of the tooth root or periodontium comprises allowing a force to be exerted on one or more teeth of a patient in need thereof, administering vitamin D to the patient and administering an effective amount of light to oral or maxillofacial bone, soft tissue, or muscle, or one or more teeth of the patient, wherein the light is administered before, during, or after the force is exerted. In some embodiments, methods for preventing or minimizing inflammation of tissue surrounding one or more teeth upon which forces are or were exerted are provided and comprise allowing a force to be exerted on one or more teeth of a patient in need thereof, administering vitamin D to the patient and administering an effective amount of light to oral or maxillofacial bone, muscle, or soft tissue, or one or more teeth of the patient, wherein the light is administered before, during, or after the force is exerted.

Bone remodeling is generally necessary for tooth movement. Accordingly, the invention further provides methods for regulating tooth movement. In one embodiment, methods for regulating tooth movement comprise administering an effective amount of vitamin D and an effective amount of light to a patient in need thereof. Light can be administered to the patient in any manner described herein. Vitamin D can likewise be administered to the patient in any manner described herein. In some embodiments, the method for regulating tooth movement comprises allowing an orthodontic appliance to exert a force on an oral or maxillofacial region of the patient. In some embodiments, the method does not comprise administering an effective amount of vitamin D to the patient. In other embodiments, the method does not comprise administering an effective amount of light to the patient.

In some embodiments, a functional appliance can cause tooth movement by exerting one or more forces on the teeth. One or more teeth, or one or more groups of teeth can move as an oral or maxillofacial bone remodels and changes orientation or position. In some embodiments, the methods for oral or maxillofacial bone remodeling are useful to increase the rate of tooth movement. In some embodiments, a functional appliance can be installed on one or more of the patient's teeth. An orthodontic appliance can be installed on one or more of the patient's teeth after the functional appliance is installed on the patient's teeth. An orthodontic appliance can be installed on one or more of the patient's teeth after the functional appliance is removed the patient's teeth. The orthodontic appliance can cause tooth movement by exerting forces on the teeth.

The methods described herein are useful for repositioning a mandibular bone. Such repositioning can comprise moving the mandibular bone forward in an anterior direction or moving it backward in a posterior direction. The methods described herein are also useful for moving the maxillary bone or mandibular bone forward or backward, lengthening or shortening the maxillary bone or mandibular bone, or adjusting the angle of the mandibular bone or maxillary bone. In some instances, repositioning or moving a bone can cause muscle tension on joints and other areas of the body. For example, in embodiments where the mandibular bone is repositioned or moved forward using a functional appliance, the bone movement causes muscle tension on the mandibular joint area, or other parts of the mandibular bone. This tension can stimulate osteoblastic activity and bone remodeling, which can lengthen the mandibular bone through bone deposition on the condylar head and glenoid fossa of the temporal bone of the skull. The condyle can have bone deposited on its distal portion and the glenoid fossa can have increased bone at the posterior which serves to change the shape of the temporomandibular joint and cause the mandibular bone to be repositioned permanently as a result. For example, a functional appliance, such as a Herbst appliance, can position a mandible forward by applying force from upper molars to lower molars, creating a muscle tension. Force can be applied to the jaw through the teeth which can be readily manipulated with fixed and removable appliances. In another example, an intra-osseous anchorage such as a titanium mini-implant can exert a force on the mandibular bone or the maxillary bone.

In some embodiments, regulating oral or maxillofacial bone remodeling further comprises using functional jaw orthopedics. Functional jaw orthopedics is a treatment with functional appliances making use of forces created by the head and neck musculature to bring about desired dental, facial, or functional changes. In functional orthopedics, generally, the muscles or tissue of the patient are used to provide orthodontic forces. A functional appliance therefore functions by exerting a force that causes muscle or tissue to exert a force directly on, for example, a tooth such that some aspect of the tooth changes as a result of said force from the muscle or tissue. In one specific example, a patient can wear a functional appliance to reposition his or her jaw, and the resultant position of the jaw exerts a force on surrounding tissue thereby allowing remodeling to occur. Functional changes can include changes in the maxillary bone, the mandibular bone, tooth position, bine and jaw function, and chewing. In contrast to functional appliances, orthodontic appliances function by exerting a force directly on, for example, a tooth to change some aspect of the tooth (e.g., to change the position of the tooth relative to another tooth).

Functional appliances can be fixed, removable, or a combination of fixed and removable. Functional appliances can alter the posture of the mandibular bone and transmit the forces created by the resulting stretch of muscles and soft tissues, and by the change in the neuromuscular environment to the dental and skeletal tissues to produce movement of the teeth and modification to the growth of the jaws and lower face. In some embodiments, regulating oral or maxillofacial bone remodeling comprises regulating a change in oral or maxillofacial bone volume or geometry.

In some embodiments, the force can be an orthopedic force. In some embodiments, an orthopedic force is a force having a magnitude of greater than about 300 grams of force. In other embodiments, an orthopedic force is a force having a magnitude of greater than or equal to about 350 grams of force, greater than or equal to about 400 grams of force, greater than or equal to about 450 grams of force, greater than or equal to about 500 grams of force, greater than or equal to about 550 grams of force, or greater than or equal to about 600 grams of force. In other embodiments, an orthopedic force is a force having a magnitude of less than or equal to about 500 grams of force, less than or equal to about 550 grams of force, less than or equal to about 600 grams of force, less than or equal to about 650 grams of force, less than or equal to about 700 grams of force, less than or equal to about 800 grams of force, less than or equal to about 900 grams of force, or less than or equal to about 1000 grams of force. In other embodiments, an orthopedic force ranges from about 300 grams of force to about 1000 grams of force. In other embodiments, an orthopedic force's lower range is about 300 grams of force, about 350 grams of force, about 400 grams of force, about 500 grams of force, about 600 grams of force or about 700 grams of force. In other embodiments the orthopedic force's upper range is about 500 grams of force, about 550 grams of force, about 600 grams of force, about 650 grams of force, about 700 grams of force, about 800 grams of force, about 900 grams of force, or about 1000 grams of force. In other embodiments, a force that is less than an orthopedic force is exerted on one or more of a patient's teeth. In this embodiment, the force has a magnitude of less than 100 grams of force, for example, a magnitude of about 200 grams of force or about 300 grams of force.

In some embodiments, the magnitude of force is the amount of force exerted on bone. For example, the magnitude of an orthopedic force can refer to the amount of force exerted per tooth. Alternatively, the magnitude of an orthopedic force can refer to the amount of force exerted on a plurality of teeth. The magnitude of force exerted per tooth in the latter instance is the total magnitude of force divided by the number of teeth. For example, if about 600 grams of force are exerted on to two teeth, then the force exerted on each tooth is about 300 grams. In some embodiments, the magnitude of an orthopedic force is the amount of force exerted on oral or maxillofacial bone, muscle, or soft tissue, or one or more teeth of a patient. In some embodiments, the force is exerted on a mandibular bone, maxillary bone, or temporal bone. In some embodiments, the force is exerted on a temporomandibular joint, condyle, or glenoid fossa. A gram of force is a unit of force equal to the magnitude of force exerted on one gram of mass by a force of 9.80665 m/s² (i.e., standard gravity).

In some embodiments, the force is a less-than-orthopedic force. In some embodiments, a less-than-orthopedic force is a force having a magnitude of greater than about 30 grams of force. In other embodiments, a less-than-orthopedic force is a force having a magnitude of greater than or equal to about 50 grams of force, greater than or equal to about 75 grams of force, greater than or equal to about 100 grams of force, greater than or equal to about 150 grams of force, greater than or equal to about 200 grams of force, or greater than or equal to about 250 grams of force. In other embodiments, a less-than-orthopedic force is a force having a magnitude of less than or equal to about 50 grams of force, less than or equal to about 75 grams of force, less than or equal to about 100 grams of force, less than or equal to about 150 grams of force, less than or equal to about 200 grams of force, less than or equal to about 250 grams of force, or less than or equal to about 275 grams of force. In other embodiments, a less-than-orthopedic force ranges from about 30 grams of force to about 300 grams of force. In other embodiments, a less-than-orthopedic force's lower range is about 30 grams of force, about 50 grams of force, about 75 grams of force, about 100 grams of force, about 150 grams of force, about 200 grams of force, or about 250 grams of force. In other embodiments the less-than-orthopedic force's upper range is about 50 grams of force, about 75 grams of force, about 100 grams of force, about 150 grams of force, about 200 grams of force, about 250 grams of force, or about 275 grams of force.

In some embodiments, the force is a heavy force. For example, in some embodiments, a heavy force is a force having a magnitude of greater than about 150 grams of force. In other embodiments, a heavy force is a force having a magnitude of greater than or equal to about 175 grams of force, greater than or equal to about 190 grams of force, greater than or equal to about 200 grams of force, greater than or equal to about 210 grams of force, greater than or equal to about 225 grams of force, or greater than or equal to about 250 grams of force. In other embodiments, a heavy force is a force having a magnitude of less than or equal to about 300 grams of force, less than or equal to about 350 grams of force, less than or equal to about 400 grams of force, less than or equal to about 450 grams of force, less than or equal to about 500 grams of force, less than or equal to about 550 grams of force, or less than or equal to about 600 grams of force. In other embodiments, a heavy force ranges from about 150 grams of force to about 600 grams of force. In other embodiments, the heavy force's lower range is about 175 grams of force, about 190 grams of force, about 200 grams of force, about 210 grams of force, about 225 grams of force or about 250 grams of force. In other embodiments, the heavy force's upper range is about 300 grams of force, about 350 grams of force, about 400 grams of force, about 450 grams of force, about 500 grams of force, about 550 grams of force, or about 600 grams of force. In other embodiments, a force that is less than a heavy force is exerted on one or more of a patient's teeth. In this embodiment, the force has a magnitude of less than 150 grams of force, for example, a magnitude of about 100 grams of force or about 125 grams of force. The magnitude of heavy force can refer to the amount of force exerted per tooth. Alternatively, the magnitude of heavy force can refer to the amount of force exerted on a plurality of teeth. The magnitude of force exerted per tooth in the latter instance is the total magnitude of force divided by the number of teeth. For example, if about 300 grams of force are exerted on to two teeth, then the force exerted on each tooth is about 150 grams.

In some embodiments, a heavy force is a force of sufficient magnitude to cause at least some amount of tooth-root resorption. In some embodiments, a heavy force has sufficient magnitude to have pathophysiological effects, to create a hyalinized zone or tissue death, to cause cell death, or to cause tissue inflammation when the heavy force is exerted without any other form of treatment, such as light treatment. The heavy force can be an excessive pathophysiological force. A pathophysiological force can cause necrosis or root resorption. The heavy force can also cause pressure on the periodontium that can result in ischemia, decreased blood flow, or cell death.

In some embodiments, the force is a less-than-heavy force. In some embodiments, a less-than-heavy force is a force having a magnitude of greater than about 10 grams of force. In other embodiments, less-than-heavy force is a force having a magnitude of greater than or equal to about 20 grams of force, greater than or equal to about 30 grams of force, greater than or equal to about 40 grams of force, greater than or equal to about 50 grams of force, greater than or equal to about 60 grams of force, greater than or equal to about 75 grams of force, or greater than or equal to about 100 grams of force. In other embodiments, less-than-heavy orthopedic force is a force having a magnitude of less than or equal to about 30 grams of force, less than or equal to about 40 grams of force, less than or equal to about 50 grams of force, less than or equal to about 60 grams of force, less than or equal to about 70 grams of force, less than or equal to about 85 grams of force, less than or equal to about 100 grams of force, or less than about 150 grams of force. In other embodiments, a less-than-heavy force ranges from about 10 grams of force to about 150 grams of force. In other embodiments, a less-than-heavy force's lower range is about 10 grams of force, about 20 grams of force, about 30 grams of force, about 40 grams of force, about 50 grams of force, about 60 grams of force, about 75 grams of force, or about 100 grams of force. In other embodiments the less-than-heavy force's upper range is about 30 grams of force, about 40 grams of force, about 50 grams of force, about 60 grams of force, about 70 grams of force, about 85 grams of force, about 100 grams of force, or less than about 150 grams of force. Additional details regarding heavy forces are described in the commonly-owned PCT Application Publication No. WO 12/048423, published Apr. 19, 2012, entitled “Method and Apparatus for Tooth Regulation with Heavy Forces,” which is incorporated herein in its entirety.

The force can be applied to a patient's oral or maxillofacial bone, muscle, or soft tissue, or to one or more teeth. In some embodiments, the force is exerted in a posterior or anterior direction relative to the patient. In some embodiments, the force is exerted normal (e.g., orthogonal or 90 degrees) relative to a side of a bone, such as an oral or maxillofacial bone (e.g., a maxillary bone, mandibular bone, or temporal bone). In some embodiments, the force is exerted at an angle relative to a posterior direction, an anterior direction, or a side of an oral or maxillofacial bone, such as a maxillary bone, mandibular bone, or temporal bone. For example, the force can be exerted at an angle of about 45 degrees, about 60 degrees, about 70 degrees, about 75 degrees, about 80 degrees, about 85 degrees, about 90 degrees, about 95 degrees, about 100 degrees, about 105 degrees, about 110 degrees, about 120 degrees, or about 135 degrees relative to a posterior direction, an anterior direction, or an oral or maxillofacial bone, such as a side of a maxillary bone, mandibular bone, or temporal bone. A force can be exerted normal (e.g., orthogonal or 90 degrees) to, downwards to, or upwards to an oral or maxillofacial bone, such as a maxillary bone, mandibular bone, or temporal bone at any angle. In some embodiments, a proximal force is applied to an oral or maxillofacial bone, such as a maxillary bone, mandibular bone, or temporal bone. In some other embodiments, a distal force is applied to an oral or maxillofacial bone, such as a maxillary bone, mandibular bone, or temporal bone. In some embodiments, a force is exerted on a mesial (e.g., towards front of mouth) side of an oral or maxillofacial bone, such as a maxillary bone, mandibular bone, or temporal bone. In some embodiments, a force is exerted on a distal (e.g., towards back of mouth) side of a maxillary bone, mandibular bone, or temporal bone. A force can be exerted on a buccal (e.g., towards cheek) side of an oral or maxillofacial bone, such as a maxillary bone, mandibular bone, or temporal bone, or a force can be exerted on a lingual (e.g., towards tongue) side of an oral or maxillofacial bone, such as a maxillary bone, mandibular bone, or temporal bone. In some embodiments, a force is applied to a temporomandibular joint (TMJ), condyle, or glenoid fossa.

A force can be applied to one or more teeth. In some embodiments, the force is exerted normal (e.g., orthogonal or 90 degrees) relative to a side of one or more teeth. In some embodiments, the force is exerted at an angle relative to a side of one or more teeth. For example, the force can be exerted at an angle of about 45 degrees, about 60 degrees, about 70 degrees, about 75 degrees, about 80 degrees, about 85 degrees, about 90 degrees, about 95 degrees, about 100 degrees, about 105 degrees, about 110 degrees, about 120 degrees, or about 135 degrees relative to a bone, such as an oral or maxillofacial bone, or relative to a side of one or more teeth. A force can be exerted normal (e.g., orthogonal or 90 degrees) to, downwards to, or upwards to one or more teeth at any angle. In some embodiments, a proximal force is applied to one or more teeth. In some other embodiments, a distal force is exerted in bone, such as oral or maxillofacial bone or to one or more teeth. In some embodiments the force is coronal pressure, which is useful to intrude teeth; in other embodiments the force is apical pressure, which is useful to extrude teeth. In some embodiments, a force is exerted on a mesial (e.g., side of tooth towards front of mouth) side of the tooth. In some embodiments, a force is exerted on a distal (e.g., side of tooth towards back of mouth) side of the tooth. A force can be exerted on a buccal (e.g., side of tooth towards cheek) side of the tooth, or a force can be exerted on a lingual (e.g., side of tooth towards tongue) side of the tooth. A force can be exerted on an occlusal surface of a tooth. A force can be exerted on an incisal surface of a tooth. A force can be exerted on a proximal (mesial/distal surfaces in between teeth) surface of a tooth. A force can be exerted on an apical (e.g., toward a root end) surface of a tooth. In some embodiments, a force exerted on a tooth is translated to be exerted on the mandibular bone or maxillary bone. The force can be exerted by a functional appliance for regulating oral or maxillofacial bone remodeling. In some embodiments, the force can be exerted by an orthodontic appliance for regulating tooth movement.

A force can be directed to move a mandibular bone or maxillary bone forward in an anterior direction. A force can be directed to move a mandibular bone or maxillary bone backward in a posterior direction. A force can be directed to adjust an angle of a mandibular bone or maxillary bone. For example, the angle of a mandibular bone can be adjusted by moving a right side or a left side of a mandibular bone forward or backward. If a right side of a mandibular bone is moved forward or lengthened, while the left side of the mandibular bone maintains the same position or is moved backward or shortened, the mandibular bone can be angled more leftward (e.g., shifted sideways or to the left side). In other words, a force can be directed to move one or more teeth toward a side. A force can also be directed to push one or more teeth toward one another or to push one or more teeth apart.

In some embodiments, a force is exerted at any point or region along an oral or maxillofacial bone, muscle, soft tissue, or one or more teeth. In some embodiments, a force is exerted at or near the top of one or more teeth, i.e., the side of a tooth opposite its root or roots. In some embodiments, a force is exerted at or near the middle of the clinical crown (e.g., exposed to the air, above the gums) of one or more teeth. In other embodiments, a force is exerted at or near the bottom of the clinical crown of one or more teeth, i.e., the clinical crown of a tooth closer to its root. In some embodiments, the force is applied to the root of the one or more teeth. A force can be exerted on one or more of the points or regions described herein on one or more teeth. In some embodiments, a force is exerted along the side of the tooth. In some embodiments, however, a force is exerted at or near a temporomandibular joint, condyle, or glenoid fossa. In some embodiments, a force is exerted on one or more of the right temporomandibular joint, right condyle, or right glenoid fossa; one or more of the left temporomandibular joint, left condyle, or left glenoid fossa; or one or more of both right and left temporomandibular joints, both right and left condyles, and both right and left glenoid fossa. In some embodiments, the force is exerted on the right temporomandibular joint without being exerted on the left temporomandibular joint, the right condyle without being exerted on the left condyle, the right glenoid fossa without being exerted on the left glenoid fossa, the left temporomandibular joint without being exerted on the right temporomandibular joint, the left condyle without being exerted on the right condyle, or the left glenoid fossa without being exerted on the right glenoid fossa. In some embodiments, the force is exerted on mandibular or maxillary alveolar bone. In some embodiments, the force is exerted on an anterior portion of the maxillary bone, mandibular bone, or temporal bone.

Depending on where or for how long the force is exerted, some or no tipping can occur to the tooth. A force can increase the velocity of tooth movement as compared to where no force or a lighter force is exerted. Exertion of a force on the maxillary bone, mandibular bone, temporal bone, or one or more teeth, particularly where the patient is administered with an effective amount of light to his or her maxillary bone, mandibular bone, temporal bone, or one or more teeth can reduce the amount of time of orthodontic treatment that a patient might undergo.

In some embodiments, a force is exerted on one or more teeth of a patient by one or more orthodontic appliances. A functional appliance, for example, can be present on one or more of the patient's teeth, other oral regions of the patient, or the patient's head or face. In some embodiments, the functional appliance exerts a force on oral or maxillofacial bone, muscle, soft tissue, or one or more teeth. The functional appliance can exert a force on only the mandibular bone of the patient. Alternatively, the functional appliance can exert a force only the maxillary bone of the patient. In some embodiments, the functional appliance exerts a force on only the temporal bone of the patient. The functional appliance can exert a force on both the mandibular bone and maxillary bone of the patient. The functional appliance can optionally exert a force on a maxillary bone, mandibular bone, or temporal bone by exerting a force on one or more tooth of the patient. The functional appliance can exert a force on only the jaw muscle. The functional appliance can exert a force on only the jaw soft tissue. The functional appliance can exert a force on only one tooth of the patient. Alternatively, the functional appliance can exert a force on a plurality of teeth of the patient. In another embodiment, the functional appliance can selectively exert a force on less than all the teeth of the patient. The functional appliance can exert a force on one or more teeth of the patient and at least one of the maxillary bone, mandibular bone, or temporal bone of the patient. In some embodiments, a functional appliance can be used for external anchorage, and can be in the form of a temporary anchorage device or in the form of headgear. In some embodiments, the functional appliance or a portion of the functional appliance can be external to the patient's oral cavity. External anchorage can be used to facilitate the exertion of forces to prevent untoward movement of anchorage teeth during use of forces.

In some embodiments, a force is exerted on one or more teeth of a patient by one or more orthodontic appliances. The orthodontic appliance can be present on one or more of the patient's teeth. In some embodiments, the orthodontic appliance exerts a force on one or more teeth. The orthodontic appliance can exert a force on only one tooth of the patient. Alternatively, the orthodontic appliance can exert a force on a plurality of teeth of the patient. In another embodiment, the orthodontic appliance can selectively exert a force on less than all the teeth of the patient.

The patient can wear a functional appliance subsequent to initiating the administration of light. In some embodiments, a force is exerted on oral or maxillofacial bone, muscle, or soft tissue, or one or more teeth of the patient subsequent to initiating the administration of light. In some embodiments, a force is exerted on the oral or maxillofacial bone, muscle, or soft tissue, or one or more teeth of the patient during the administration of light. In some embodiments, a force is exerted on the oral or maxillofacial bone, muscle, or soft tissue, or one or more teeth of the patient prior to initiating the administration of light. For example, the force can be exerted on the oral or maxillofacial bone, muscle, or soft tissue, or one or more teeth of the patient at least about 1 day, 2 days, 3 days, 4 days, 5 days, 6 days, 7 days, 2 weeks, or 3 weeks prior to initiating the administration of light. A force can be exerted on the oral or maxillofacial bone, muscle, or soft tissue, or one or more teeth of the patient from any direction. In some embodiments, the force moves the mandibular bone forward or backwards relative to the maxillary bone, or the maxillary bone forward or backwards relative to the mandibular bone. In some embodiments, the force pushes two or more teeth together or apart, or pushes one or more teeth to one side or area of a patient's mouth.

Regulating oral or maxillofacial bone remodeling can comprise changing the position of the mandibular bone or maxillary bone relative to one another or to the skull of the patient. Regulating oral or maxillofacial bone remodeling can also comprise controlling the position (e.g., forward, backward, sideways or angle) of the mandibular bone or maxillary bone, lengthening or shortening the mandibular bone or maxillary bone, lengthening or shortening a side of the mandibular bone or maxillary bone, altering the shape or dimensions of the mandibular bone or maxillary bone, or regulating (e.g., increasing, decreasing or maintaining) the velocity of the movement of the mandibular bone or maxillary bone relative to one another. For example, regulating oral or maxillofacial bone remodeling can comprise increasing the velocity of oral or maxillofacial bone remodeling.

By repositioning a mandibular bone forward or backwards, muscle tension can be caused on the joint area of the mandibular bone, or other parts of the mandibular bone. This tension can stimulate osteoblastic activity or bone remodeling, which can lengthen the mandibular bone through bone deposition on the condylar head and glenoid fossa of the temporal bone of the skull. Also, the tension can effect dental movement forward of the entire lower arch. In some cases, antagonistic force on the maxillary bone can retard the growth of the maxillary bone and cause remodeling and dental movement posteriorly. This can be desirable in situations where the oral or maxillofacial bone remodeling is regulated in order to remodel the maxillary bone posteriorly. Malocclusion can exist when there is a misalignment of teeth or the upper dental arch and the lower dental arch do not line up. The antagonist force on the maxillary bone can be more or less desirable depending on the severity of the malocclusion and whether the maxillary bone is protrusive. If the maxillary bone is protrusive, it can be desirable to retard maxillary forward growth or even retrude maxillary teeth and the jaw bone. A maxillary headgear can be used to retard or decrease the growth of the maxilla forward. In one example, a functional appliance can be used to reposition a mandibular bone forward while utilizing upper teeth or the maxillary bone as anchorage. An equal and opposite force can be exerted on the maxillary bone, which can lead to dental orthodontic movement and bone remodeling on the maxillary bone.

Some functional appliances (e.g., Bionator or Frankel), can prevent antagonist muscles from pushing on the bone and teeth. This can permit opposite agonist muscles to push on the bone and teeth. Thus, in some embodiments, allowing a force to be exerted on an oral or maxillofacial bone, muscle, or soft tissue, or one or more teeth, can comprise preventing a first group of muscles from exerting a force on the oral or maxillofacial bone, muscle, or soft tissue, or one or more teeth, thereby allowing a second group of muscles to exert the force. Some examples of muscles whose forces can be withheld, include cheek and lip (peri-oral) muscles. Examples of such muscles can include masseters, buccinators, mentalis muscle and orbicularis. This can allow other muscles, such as the tongue, to exert a force on the oral or maxillofacial bone, muscle, or soft tissue, or one or more teeth. In some cases, it can be desirable to prevent the tongue from interfering with and pushing on teeth, so a functional appliance or orthodontic appliance can be inserted to prevent the tongue from pushing on the front teeth during swallowing. This could allow cheek and lip muscles to push on teeth and bone to retract and allow teeth to erupt into a normal position previously presented by an overactive and poorly positioned tongue. In one example, a Frankel appliance can hold the cheek and lip muscles away from the teeth to allow them room to grow into the correct position. While the cheek and lip muscles (opposing muscles) are held away from the teeth, the tongue (an agonist muscle pushing against the teeth from the inside) can push on the teeth, thereby allowing a lower arch, upper arch, or both lower and upper arch to expand without interference from the opposing cheek and lip muscles.

In some embodiments, the force exerted by a functional appliance can prevent muscles of a first group from exerting a first force, or can substantially reduce the amount of the first force, allowing muscles in a second group to exert a second force, which can result in bone remodeling caused by the second force. The muscles in the first group and the muscles in the second group can typically exert forces in different directions. For example, muscles can exert forces anteriorly, posteriorly, laterally to the left, laterally to the right, radially inward, radially outward, upward, or downward. In some embodiments, the muscles of the first group and the muscles of the second group can exert forces in a substantially opposite direction. The muscles in the first group and the muscles in the second group can exert forces in different directions. Alternatively, the force exerted by the functional appliance can alter the angle of the overall force applied to the region by increasing the relative effect of the second force, which can result in bone remodeling caused by the increased magnitude on the second force relative to the first force. Any number of muscle groups (e.g., 1, 2, 3, 4, 5, 6, or more) can exert force in any direction. The force exerted by the functional appliance can prevent one or more of the muscle groups from exerting a force or can reduce the amount of force exerted by one or more groups.

In some embodiments, a functional appliance can keep muscles away from the teeth so that the muscles that oppose those that are withdrawn via the functional appliance then can exert forces on the teeth to cause tooth movement and possible bone remodeling due to “imbalance” of previously balanced muscular pressure. In some embodiments, the functional appliance exerts a force on the oral or maxillofacial muscle or soft tissue in order to keep the muscles away.

In some embodiments, regulating bone remodeling can also comprise regulating tooth movement. Regulating tooth movement can comprise controlling the position of one or more teeth relative to a supporting tissue. Regulating tooth movement can also comprise controlling (e.g., increasing, decreasing, maintaining) the velocity of tooth movement relative to a supporting tissue. For example, regulating tooth movement can comprise increasing the velocity of tooth movement. Regulating tooth movement can also comprise controlling (e.g., increasing, decreasing, maintaining) bodily movement (e.g., less tipping, more tipping) of one or more teeth. Regulating tooth movement can comprise moving one or more teeth bodily. “Bodily” movement can occur when the tooth is generally perpendicular to the bone, versus “tipped” movement, wherein the crown or coronal region of the tooth advances more quickly than the root or apical region of the tooth. Bodily tooth movement can comprise moving a tooth without causing significant tipping of the tooth. By “significant tipping” is meant that about 20% of the tooth does not move in the same lateral direction as the remaining about 80%; in another embodiment about 10% of the tooth does not move in the same lateral direction as the remaining about 90%, in another embodiment about 5% of the tooth does not move in the same lateral direction as the remaining about 95%. Tooth movement can include lateral displacement of one or more teeth. Regulating tooth movement can comprise inducing the tilting or tipping one or more teeth, minimizing or preventing the tilting or tipping one or more teeth, or maintaining an alignment or orientation of the one or more teeth. Regulating tooth movement can also comprise stabilizing tooth movement. In some embodiments, regulating tooth movement can comprise causing one or more teeth to maintain their position. In some embodiments, regulating tooth movement can include a combination of causing the displacement of one or more teeth and causing one or more other teeth to maintain their position.

Light can be administered inter-orally or extra-orally. Light can be administered to a region of the patient's oral or maxillofacial bone, muscle, or soft tissue, or one or more teeth. In some embodiments, light is administered to the maxillary bone, mandibular bone, or temporal bone, or other region of the patient. In some embodiments, the light can be directed to one or more regions of a patient. The region can be within the patient's mouth. The region can be all or a portion of the patient's maxillary bone, mandibular bone, or temporal bone of the skull. The region can be a temporomandibular joint, condyle, or glenoid fossa of the patient. The region can be the right temporomandibular joint, right condyle, or right glenoid fossa; left temporomandibular joint, left condyle, or left glenoid fossa; or both temporomandibular joints, both condyles, or both glenoid fossa of the patient.

Light can be administered to a right temporomandibular joint without being administered to a left temporomandibular joint, a right condyle without being administered to a left condyle, a right glenoid fossa without being administered to a left glenoid fossa, a left temporomandibular joint without being administered to a right temporomandibular joint, a left condyle without being administered to a right condyle, or a left glenoid fossa without being administered to a right glenoid fossa. The region can include a portion of the maxillary bone (e.g., portion of the patient's maxillary alveolar bone), a portion of the mandibular bone (e.g., portion of the patient's mandibular alveolar bone), or alveolar mucosa.

In some embodiments, in addition to being administered to a region of the patient's maxillary bone, mandibular bone, or temporal bone, light can be administered to other regions of the patient. Such regions can include, but are not limited to, one or more teeth (e.g., incisor, canine, premolar, or molar, such as a maxillary central incisor, maxillary lateral incisor, maxillary canine, maxillary first premolar, maxillary second premolar, maxillary first molar, maxillary second molar, maxillary third molar, mandibular central incisor, mandibular lateral incisor, mandibular canine, mandibular first premolar, mandibular second premolar, mandibular first molar, mandibular second molar, or mandibular third molar), a root of one or more teeth (e.g., wherein a root of a tooth can include a portion of one or more roots supporting the tooth, one root supporting the tooth, a plurality of roots supporting the tooth, or all of the roots supporting the tooth), tissue supporting one or more teeth, basal tissue, gingiva, periodontal ligaments, cementum, periodontium, a region of jaw bone or tissue, or at least a portion of the patient's other oral soft tissue or bone tissue. The region can be located on a left side or right side of the patient's face. In some embodiments, one or more regions are located on both the left and right side of the patient's face. In some embodiments, the region can be located on the front side of the patient's face. The region can include one, two, three, four, five, six, seven, eight or more teeth, or tissue surrounding or supporting the teeth. The region can include one or more roots of one, two, three, four, five, six, seven, eight, or more teeth, or periodontium of teeth. In other embodiments, light is not administered to a region outside the patient's maxillary bone, mandibular bone, or temporal bone. In some embodiments, light is not administered to a region outside the patient's oral or maxillofacial bone, muscle, or soft tissue, or one or more teeth. In some embodiments, the region includes the patient's skull, spine, pelvis or femur.

Light can be administered to regions that can include tissue (e.g., alveolar or basal tissue) surrounding or supporting any of the teeth specifically described with or without including the tooth itself. Regions can include teeth or tissue supported by the maxillary bone or teeth supported by the mandibular bone. One or more regions can be adjacent to one another, continuous with one another, or separate from one another. Any discussion herein of regions or examples of regions can apply to any other region or examples of treatment regions provided herein.

In some embodiments, light irradiates a region that can include a portion of tissue (e.g., bone tissue, or soft tissue) or other regions within the patient's oral cavity without irradiating one or more other portions of the patient's oral cavity. In some embodiments, light is administered, directly or indirectly, to one or more temporomandibular joint, condyle, or glenoid fossa of the patient. In some embodiments, light is administered, directly or indirectly, to only one temporomandibular joint, only one condyle, or only one glenoid fossa of the patient. In some embodiments, light is administered, directly or indirectly, to one or more temporomandibular joint, condyle, or glenoid fossa of the patient, without being administered to other regions of the patient's oral cavity, or without being administered to one or more of the patient's teeth, or without being administered to any of the patient's teeth. In some embodiments, light is administered, directly or indirectly, to one or more roots of only one tooth root and to only one periodontium. Alternatively, light is administered, directly or indirectly, to one or more roots of a plurality of teeth and to a plurality of periodontia. Light can be administered, directly or indirectly, to one or more roots of all or less than all the teeth and periodontia in the patient's oral cavity. One or more selected teeth, roots or periodontia can be irradiated, directly or indirectly, with light.

In some embodiments, light irradiates a region that can include a portion of tissue (e.g., bone tissue, or soft tissue) or other regions within the patient's oral cavity at a much greater intensity than it irradiates other portions of the patient's oral cavity. For example, light can irradiate a region at an intensity that is 3×, 5×, 10×, 20×, 50×, or 100× greater than the intensity that irradiates any another region. In some embodiments, the region is the patient's oral cavity or a portion thereof. In some embodiments, light irradiates a portion of a patient's oral or maxillofacial bone, muscle, or soft tissue, or one or more teeth at a greater intensity than that of light that irradiates another portion of the patient's oral or maxillofacial bone, muscle, or soft tissue, or one or more teeth. In one embodiment, light irradiates a portion of a patient's maxillary bone, mandibular bone, or temporal bone, such as the temporomandibular joint, condyle, or glenoid fossa, at a greater intensity than that of light that irradiates any of the patient's teeth. In another embodiment, light irradiates or is focused with a greater intensity on the region where forces are exerted, relative to the region where forces are not exerted. Teeth with lower forces or anchorage teeth can be selectively shielded from light or irradiated at lower light intensity so that they can move less and the anchorage effect can be enhanced. In some embodiments, this is achieved by applying to the patient, or adjusting within the patient, one or more intra-oral or extra-oral light-translucent or light-opaque masks that shield from light one or more non-regions. In some embodiments, light reaching a region has an intensity that is greater than a threshold value. In some embodiments, the threshold value has an intensity as described herein.

The region can be close to a surface within the patient's mouth, or within a soft tissue or bone tissue. The region can be at a depth from the surface of the patient's skin, such as the patient's face. For example, the region can be about 1 nm, about 1 μm, about 10 μm, about 50 μm, about 100 μm, about 200 μm, about 300 μm, about 500 μm, about 750 μm, about 1 mm, about 2 mm, about 3 mm, about 4 mm, about 5 mm, about 7 mm, about 10 mm, about 15 mm, about 20 mm, about 25 mm, about 30 mm, about 40 mm, about 50 mm, about 60 mm, or about 70 mm from the surface of the patient's skin. Light can irradiate a region, which can have an area greater than, less than, or about 1 nm², about 1 μm², about 0.1 mm², about 0.2 mm², about 0.3 mm², about 0.4 mm², about 0.5 mm², about 0.7 mm², about 1 mm², about 10 mm², about 0.2 cm², about 0.5 cm², about 1 cm², about 2 cm², about 3 cm², about 5 cm², about 7 cm², about 10 cm², about 15 cm², about 20 cm², about 25 cm², about 30 cm², about 35 cm², about 40 cm², about 50 cm², about 60 cm², about 80 cm², about 100 cm², about 120 cm², about 140 cm², about 160 cm², about 180 cm² or about 200 cm². Light can irradiate one area, a plurality of areas, a point, or a plurality of points. In some embodiments, light irradiates a particular area without irradiating with significant intensity surrounding areas. For example, light can irradiate a portion of maxillary bone, mandibular bone, or temporal bone without significant amounts of light irradiating teeth on that maxillary bone, mandibular bone, or temporal bone. In one embodiment, the light irradiates a temporomandibular joint, condyle, or glenoid fossa without significant amounts of light irradiating teeth on that maxillary bone, mandibular bone, or temporal bone or other regions of the maxillary bone, mandibular bone, or temporal bone. In another embodiment, light irradiates a particular tooth or set of teeth without significant amounts of light irradiating adjacent teeth. In one embodiment, irradiating a tooth comprises irradiating an exposed surface of the tooth, a tooth root, or a periodontium of the tooth.

In some embodiments, light is administered extra-orally to the patient. Light can be emitted from a light source that contacts the patient's skin. The light source can contact the skin of the patient overlying a region where bone remodeling regulation is intended to occur. In some embodiments, the light source can contact the skin of the patient at the face, neck, torso, arms, or legs of the patient. In some embodiments, light is provided from a light-therapy apparatus, embodiments of which are described below. Light can be emitted from a light source that can include characteristics, features, components, or configurations of any of the light-therapy apparatus embodiments, as described below. The present methods can further comprise providing a light-therapy apparatus. For example, the method for regulating oral or maxillofacial bone remodeling can comprise administering light from a light-therapy apparatus. Light can be provided from any other source, and is not limited to a light-therapy apparatus as described herein.

In some embodiments, light is provided from a light source that can contact the patient's skin (e.g., face). Similarly, light can be emitted from a plurality of light sources that can contact the patient's face. In one embodiment, one or more light sources contact skin of the patient's face overlying a region. For example, one or more light sources can contact skin of the patient's face overlying a portion of a maxillary bone, mandibular bone, or temporal bone, such as a temporomandibular joint, a condyle, or a glenoid fossa. In other words, in some embodiments, the one or more light sources are positioned directly over a right temporomandibular joint, a left temporomandibular joint, a right condyle, a left condyle, a right glenoid fossa, or a left glenoid fossa of the patient. The one or more light sources can contact the skin of the patient overlying a region where bone remodeling or tooth movement regulation is intended to occur. Light can be administered from a light source that can provide pressure on the patient's face. Light can pass through the patient's face to irradiate the region. The region can be located within a patient's oral cavity. In some embodiments, a light emitter is provided externally to the oral cavity. A portion of a patient's face, such as the cheek, skin over the jaw, lips, or chin can be located between the light emitter and the oral cavity. Light can be administered transcutaneously to a region that is located within the patient's oral cavity. The light can transcutaneously pass through the skin of the patient to irradiate the region. Light can pass through the cheek of the patient, the skin overlying the maxillary bone, mandibular bone, or temporal bone of the patient (such as skin overlying a temporomandibular joint of the patient, a condyle of the patient, a glenoid fossa of the patient), the chin of the patient, the lips of the patient, or any other region circumscribed or otherwise defined by the patient's face. In some embodiments, light irradiates a region by manually retaining one or more light sources providing light of one or more wavelengths to one or more regions of a patient. In some embodiments, light irradiates a region only transdermally through the skin of the patient. In some embodiments, light is administered only externally, and is not administered internally. For example, light can be administered only extra-orally, and can not be administered intra-orally. In some alternate embodiments, light is administered internally (e.g., intra-orally) or externally (e.g., extra-orally). In one embodiment, the patient to whom the light is administered has his or her mouth closed.

In other embodiments, the light source does not contact the patient's face or other skin. Extra-oral light can also be administered to the patient wherein a gap exists between a light source and skin of the patient's face. The light source can be in close proximity to the skin of the patient's face without contacting the patient's face. In some embodiments, light is administered from a light source that does not contact a patient's face when the patient's face is relaxed but can contact the face if the patient flexes a portion of the patient's face or tenses the face. In some embodiments, a light source is about 1 mm or less, about 2 mm or less, about 3 mm or less, about 5 mm or less, about 7 mm or less, about 1 cm or less, about 1.5 cm or less, about 2 cm or less, about 2.5 cm or less, or about 3 cm or less away from a patient's face while the patient's face is relaxed or tensed. Light can be emitted from a light source located at a particular distance from a region. In some embodiments, the distance is about 0.1 mm or less, about 0.5 mm or less, about 1 mm or less, about 2 mm or less, about 3 mm or less, about 5 mm or less, about 7 mm or less, about 1 cm or less, about 1.5 cm or less, about 2 cm or less, about 2.5 cm or less, or about 3 cm or less. In some embodiments, a light source is about 0.1 mm, about 0.5 mm, about 1 mm, about 3 mm, about 5 mm, about 7 mm, about 1 cm, about 1.5 cm, about 2 cm and about 2.5 cm, about 2.75 cm, about 3 cm, about 3.5 cm, or about 4 cm away from the region to be treated by or irradiated by an effective amount of light.

In some embodiments, light is administered intra-orally to the patient. For example, the light source can be located within the patient. In some embodiments, the light source can comprise fiber optics that convey light within the patient. In some embodiments, the light source can be located within an orifice of the patient. For example, the light source can be located within the patient's oral cavity. In some embodiments, light is administered directly to a selected region or to a surface overlaying the selected region. In some embodiments, the light source is located outside the patient's oral cavity and the light is administered directly to a selected region or to a surface overlaying the selected region. In some embodiments where light is administered directly to a selected region, the light reaches the selected region without first reflecting from another region. In some embodiments, light is administered to a selected region through the patient's gums or soft tissue. Light need not be administered transdermally or through the patient's face. In some embodiments, the light source contacts the selected region or surface overlying the selected region. For example, the light source, or a light therapy apparatus comprising the light source, can contact a patient's alveolar mucosa, tooth or gum. In some embodiments, light is directed at the selected region through soft tissue.

Light can be administered from a single light source. Alternatively, light can be administered from multiple light sources. Light can irradiate a continuous region or one or more discrete regions. Light can irradiate various regions from different directions. For example, light can be administered from one or both of a right side of a patient's body (e.g., the right side of the patient's face) and from a left side of a patient's body (e.g., the left side of the patient's face). Light can be administered so that it is angled upward toward a region, or can be administered so that it is angled downward to toward a region. In some embodiments, light is administered from one or more stationary sources. For example, a light source can remain stationary during administration. In some embodiments, light is administered from one or more moving light sources. A light source can be displaced, can be angled, can be rotated, or any combination thereof. Light can be administered from a continuously moving source, or can be administered from a discretely or abruptly moving source.

An effective amount of light can be administered. An effective amount of light is an amount of light that is effective to regulate bone remodeling or tooth movement when administered before, during or after an orthodontic appliance, e.g., a functional appliance, exerts a force on oral or maxillofacial bone, muscle or soft tissue, or one or more teeth of a patient, or before, during or after vitamin D is administered to the patient. In some embodiments, bone remodeling also results in or affect tooth movement regulation, tooth-root resorption, bone resorption, inflammatory dentin resorption, cementum resorption, tissue inflammation, or remodeling of maxillary or mandibular bone. The properties can include, but are not limited to: light intensity, light wavelength, light coherency, light range, peak wavelength of emission, light energy density, continuity, pulsing, duty cycle, frequency, duration, or whether a light emitter is on or off.

A method for regulating bone remodeling, such as oral maxillofacial bone remodeling, can further comprise determining an effective dosage of light. The determination can be based on an intended oral or maxillofacial bone remodeling regulation effect. The method can further comprise selecting on or more light properties to provide the effective dosage of light. The method can further comprise receiving instructions from a controller, and emitting light having particular properties. The controller can be any controller described herein or can implement any of the steps described herein.

Light can be administered from one or more light source capable of irradiating light having intended properties. A light source can emit light from one or more light emitters. In some embodiments, a light source comprises about 10 to about 15 emitters, about 15 to about 20 emitters, about 20 to about 30 emitters, about 30 to about 40 emitters, about 40 to about 50 emitters, about 50 to about 70 emitters, or about 70 emitters to about 100 emitters. For example, light can be administered from a light source, which can comprise one or more of the following emitters: a light-emitting diode (LED), which can be present in an array; and a laser, for example a vertical cavity surface emitting laser (VCSEL) or other suitable light emitter such as an Indium-Gallium-Aluminum-Phosphide (InGaAlP) laser, a Gallium-Arsenic Phosphide/Gallium Phosphide (GaAsP/GaP) laser, or a Gallium-Aluminum-Arsenide/Gallium-Aluminum-Arsenide (GaAlAs/GaAs) laser. In one embodiment the light source comprises a plurality of lasers. A plurality of light emitters can emit light at one or more different wavelengths. Alternatively, one or more light emitters can emit light at the same wavelength for a light source. One or more light emitters can be arranged on a light source in any manner, such as a linear array or another arrangement described herein.

An effective amount of light has an intensity that is effective in the present methods. In one embodiment, the light intensity is at least about 10 mW/cm². In other embodiments, the light intensity is about 1 mW/cm² or greater, about 3 mW/cm² or greater, about 5 mW/cm² or greater, about 7 mW/cm² or greater, about 12 mW/cm² or greater, about 15 mW/cm² or greater, about 20 mW/cm² or greater, about 30 mW/cm² or greater, about 50 mW/cm² or greater, about 75 mW/cm² or greater, about 100 mW/cm² or greater, about 200 mW/cm² or greater, about 500 mW/cm² or greater, or about 1 W/cm² or greater. In other embodiments, the light intensity is about 20 mW/cm² or less, about 30 mW/cm² or less, about 50 mW/cm² or less, about 75 mW/cm² or less, about 100 mW/cm² or less, about 200 mW/cm² or less, about 500 mW/cm² or less, about 1 W/cm² or less, about 2 W/cm² or less, about 5 W/cm² or less, or about 10 W/cm² or less. In one embodiment the light intensity ranges from about 1 mW/cm² to about 10 W/cm². In another embodiment, the light intensity's lower range is about 3 mW/cm², about 5 mW/cm², about 7 mW/cm², about 12 mW/cm², about 15 mW/cm², about 20 mW/cm², about 30 mW/cm², about 50 mW/cm², about 75 mW/cm², about 100 mW/cm², about 200 mW/cm², about 500 mW/cm², or about 1 W/cm². In another embodiment, the light intensity's upper range is about 20 mW/cm², about 30 mW/cm², about 50 mW/cm², about 75 mW/cm², about 100 mW/cm², about 200 mW/cm², about 500 mW/cm², about 1 W/cm², about 2 W/cm², about 5 W/cm², or about 10 W/cm². Light can be administered having an intensity falling within a range determined by any of the intensities disclosed herein. In some embodiments the intensity is an average intensity. In some embodiments, the light has an intensity in the range of about 10 mW/cm² to about 60 mW/cm², or about 20 mW/cm² to about 60 mW/cm². In such embodiments, the peak light intensity can about 50 mW/cm² or greater. A peak wavelength is the wavelength at which the highest intensity of light is emitted. In some embodiments, light can be pulsed. In other embodiments, the output of light is continuous. In some embodiments, the light intensity can vary over time in a cyclical or non-cyclical fashion. The light intensity can vary with or without pulsing. In some embodiments, pulse width modulation can be used to effect a desired light intensity. If one or more wavelengths of light are administered, then each wavelength can be administered at its own intensity.

In some embodiments, an effective amount of light includes light having a wavelength that is within in a particular range, or light of a range of wavelengths. The light is not necessarily visible light. For example, the light can include infrared light or near-infrared light. The light can also be provided in the visible light region. Light can be administered having one or more wavelengths ranging from about 620 nm to about 1000 nm. In some embodiments, administered light has one or more wavelengths ranging from about 585 nm to about 665 nm, about 666 nm to about 814 nm, about 815 nm to about 895 nm, about 640 nm to about 680 nm, or about 740 nm to about 780 nm, or any given wavelength or range of wavelengths within those ranges, such as, for example, about 625 nm or about 855 nm, or about 605 nm to about 645 nm, or about 835 nm to about 875 nm. In some embodiments, the administered light has one or more wavelengths from about 605 nm to about 645 nm, or from about 835 nm to about 875 nm. In some embodiments, the administered light has one or more wavelengths from about 615 nm to about 635 nm, or from about 845 nm to about 865 nm. In some embodiments, the wavelengths of the administered light is about 625 nm or about 855 nm. In additional embodiments, the administered light has one or more wavelengths ranging from about 400 nm to about 1200 nm. In particular embodiments, the administered light has one or more wavelengths ranging from about 500 nm to about 700 nm, about 585 nm to about 665 nm, about 605 nm to about 630 nm, about 620 nm to about 680 nm, about 666 nm to about 814 nm, about 815 nm to about 895 nm, about 820 nm to about 890 nm, about 640 nm to about 680 nm, or about 740 nm to about 780 nm. In some embodiments the administered light has one or more wavelengths in one or both of the following wavelength ranges: about 820 to about 890 nm and about 620 to about 680 nm. In some embodiments, the administered light has one or more wavelengths in the ranges of about 820 to about 890 nm and about 620 nm to about 680 nm. In some embodiments, the administered light has one or more wavelengths in the ranges of about 815 to about 895 nm and about 585 to about 665 nm. The administered light can alternatively have one or more wavelengths in one or more of the following ranges: about 613 nm to about 624 nm, about 667 nm to about 684 nm, about 750 nm to about 773 nm, about 812 nm to about 846 nm. In one embodiment, the light wavelength's lower range is about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm. In another embodiment, the light wavelength's upper range is about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 935 nm, about 975 nm, about 1000 nm, about 1050 nm, about 1100 nm, or about 1200 nm.

The wavelengths of light administered can be limited to any of the ranges or limits described herein. Additionally, the wavelengths of light administered with a sufficient intensity to be an effective amount can be limited to any of the ranges or limits described herein.

For example, in some embodiments, light administered to a region does not have wavelengths exceeding one or more of the following: about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm. For example, no light exceeding about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm can be administered to a selected region. In some examples, light administered to a region does not have wavelengths below one or more of the following: about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm. For example, no light below about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm is administered to a selected region. In some embodiments, the light administered does not comprise a wavelength of about 600 nm or less. In some embodiments, the light administered does not comprise a wavelength of about 1000 nm or greater. In some embodiments, the light administered does not comprise a wavelength of about 600 nm or less and does not comprise a wavelength of about 1000 nm or greater.

In some embodiments, light administered to a region with a sufficient intensity to be an effective amount in the present methods does not have wavelengths exceeding one or more of the following: about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm. For example, no light having a sufficient intensity to be an effective amount for oral or maxillofacial bone remodeling and exceeding about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm can be administered to a selected region. In some examples, light administered to a region with a sufficient intensity to be an effective amount in the present methods does not have wavelengths exceeding one or more of the following: about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm. For example, no light having a sufficient intensity to be an effective amount in the present methods and below about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm is administered to a selected region. In some embodiments, the light administered does not comprise a wavelength of about 600 nm or less having a sufficient intensity to be an effective amount for the present methods. In some embodiments, the light administered does not comprise a wavelength of about 1000 nm or greater having a sufficient intensity to be an effective amount for the present methods. In some embodiments, the light administered does not comprise a wavelength of about 600 nm or less having a sufficient intensity to be an effective amount for the present methods and does not comprise a wavelength of about 1000 nm or greater having a sufficient intensity to be an effective amount for the present methods.

In some embodiments, light is administered at one, two, or more of the light ranges described. In some embodiments, light is not administered outside of one, two, or more of the light ranges described. In some embodiments, light is not administered with a sufficient intensity to constitute an effective amount for regulating bone remodeling outside of one, two, or more of the light ranges described. In other embodiments, administered light has other wavelengths, as desired for a particular application. In some embodiments, light having a first set of characteristics (e.g., wavelength, intensity, pulsing, timing) is administered to a first region, and light with a second set of characteristics is administered to a second region. The first region and the second region can be the same region, can partially overlap, or can be different regions. The first set of characteristics can be the same as the second set of characteristics, can partially overlap with the second set, or can all be different from the second set. In one embodiment, one region of a bone (e.g., a maxillary bone, mandibular bone, or temporal bone) receives light within a first wavelength range, while another region of the bone receives light within a second wavelength range. The first and second wavelengths can overlap. Alternatively, the first and second wavelengths do not overlap.

Although examples of light wavelength ranges are provided below for different applications, light having any other light wavelength value, which can include those described herein, can be administered for those applications.

Administering to the maxillary bone, mandibular bone, or temporal bone (e.g., at a temporomandibular joint, condyle, or glenoid fossa) or to any other oral or maxillofacial bone, soft tissue, or muscle, or one or more teeth of a patient light having a wavelength in the range of about 815 nm to about 895 nm, such as about 835 nm to about 875 nm, or about 855 nm in conjunction with a functional appliance and/or administering vitamin D, is useful for increasing the rate of bone remodeling or tooth movement. In another embodiment, intra-orally administering to the maxillary bone, mandibular bone, or temporal bone, or to any other oral or maxillofacial bone, soft tissue, or muscle, or one or more teeth of a patient light having a wavelength in the range of about 815 nm to about 895 nm, such as about 835 nm to about 875 nm, or about 855 nm in conjunction with a functional appliance and/or administering vitamin D, is useful for increasing the rate of bone remodeling. In some embodiments, the soft tissue is alveolar mucosa.

Administering to the maxillary bone, mandibular bone, or temporal bone (e.g., at a temporomandibular joint, condyle, or glenoid fossa) or to any other oral or maxillofacial bone, soft tissue, or muscle, or one or more teeth of a patient light having a wavelength in the range of about 585 nm to about 665 nm, such as about 605 nm to about 645 nm, or about 625 nm in conjunction with a functional appliance and/or administering vitamin D, is useful for regulating bone remodeling. In another embodiment, intra-orally administering to the maxillary bone, mandibular bone, or temporal bone or to any other oral or maxillofacial bone, soft tissue, or muscle, or one or more teeth of a patient light having a wavelength in the range of about 585 nm to about 665 nm, such as about 605 nm to about 645 nm, or about 625 nm in conjunction with a functional appliance and/or administering vitamin D, is useful for bone remodeling. In one embodiment, administration of the light increases the rate of bone remodeling, such as oral or maxillofacial bone remodeling. In some embodiments, the soft tissue is alveolar mucosa.

Administering to the maxillary bone, mandibular bone, or temporal bone (e.g., at a temporomandibular joint, condyle, or glenoid fossa) or to any other oral or maxillofacial bone, soft tissue, or muscle, or one or more teeth of a patient light having a wavelength in the range of about 666 nm to about 814 nm is useful for increasing the rate of bone remodeling. In another embodiment, intra-orally administering to the maxillary bone, mandibular bone, or temporal bone or to any other oral or maxillofacial bone, soft tissue, or muscle, or one or more teeth of a patient light having a wavelength in the range of about 666 nm to about 814 nm in conjunction with a functional appliance and/or administering vitamin D, is useful for increasing the rate of bone remodeling. In some embodiments, the soft tissue is alveolar mucosa.

Administering to the alveolar mucosa and/or teeth of a patient light having a wavelength in the range of about 815 nm to about 895 nm, such as about 835 nm to about 875 nm, or about 855 nm in conjunction with a functional appliance and/or administering vitamin D, is useful for regulating bone remodeling and increasing the rate of movement of teeth. In another embodiment, intra-orally administering to the alveolar mucosa and teeth of a patient light having a wavelength in the range of about 815 nm to about 895 nm, such as about 835 nm to about 875 nm, or about 855 nm in conjunction with a functional appliance and/or administering vitamin D, is useful for regulating bone remodeling and increasing the rate of movement of teeth. In one embodiment administration of the light increases the rate of oral or maxillofacial bone remodeling. In some embodiments, the regulation of oral or maxillofacial bone remodeling can result in the regulation of tooth movement. In one embodiment, increasing the rate of tooth movement does not increase the tipping motion of teeth beyond that which is experienced by orthodontic patients who are not provided with light.

Administering to the alveolar mucosa and/or teeth of a patient light having a wavelength in the range of about 585 nm to about 665 nm, such as about 605 nm to about 645 nm, or about 625 nm in conjunction with a functional appliance and/or administering vitamin D, is likewise useful for regulating bone remodeling. In another embodiment, intra-orally administering to the alveolar mucosa and teeth of a patient light having a wavelength in the range of about 585 nm to about 665 nm, such as about 605 nm to about 645 nm, or about 625 nm in conjunction with a functional appliance and/or administering vitamin D, is likewise useful for regulating bone remodeling. In one embodiment, administration of the light increases the rate of tooth movement.

In one embodiment administration of light having a wavelength in the range of about 585 nm to about 665 nm, in conjunction with a functional appliance and/or administering vitamin D increases the amount or extent of bodily tooth movement to a greater degree than administration with light having a wavelength in the range of about 815 nm to about 895 nm. Administering light having a wavelength in the range of about 585 nm to about 665 nm (e.g., about 625 nm) can result in about 10% to about 50% less tipping than the administration of light having a wavelength in the range of about 815 nm to about 895 nm (e.g., about 855 nm). For example, about 10%, about 15%, about 20%, about 25%, about 30%, about 35%, about 40%, about 45%, or about 50% less tipping can occur. Particular wavelengths of light can minimize tipping.

Thus, in one embodiment administration of light having a wavelength in the range of about 605 nm to about 645 nm, such as about 625 nm, in conjunction with a functional appliance, is useful for facilitating the bodily movement of teeth in orthodontic treatment and optionally increase bone remodeling. In some embodiments the methods further comprise increasing bone remodeling. In another embodiment administration of light having a wavelength in the range of about 835 to about 875 nm, such as about 855 nm, is useful for increasing the rate of movement of teeth for which some degree of tipping movement is desirable or acceptable and also for regulating bone remodeling.

In other embodiments administration of light having a wavelength in the range of about 605 nm to about 645 nm, such as about 625 nm, in conjunction with a functional appliance and/or administering vitamin D, is useful for increasing the quality or degree of bone remodeling, such as oral or maxillofacial bone remodeling. Accordingly the present invention further relates to methods for increasing the quality or degree of oral or maxillofacial bone remodeling, comprising extra-orally administering to a patient in need thereof an effective amount of light transdermally to a region of the patient's oral or maxillofacial bone, muscle, or soft tissue or one or more teeth, such as a maxillary bone, mandibular bone, temporal bone, or other regions as described herein. In other embodiments, the present invention further relates to methods for increasing the quality or degree of oral or maxillofacial bone remodeling, comprising intra-orally administering to a patient in need thereof an effective amount of light to a region of the patient's oral or maxillofacial bone, muscle, or soft tissue, or one or more teeth, such as a maxillary bone, mandibular bone, temporal bone, or other regions as described herein. For example, light can be administered to regions of one or both temporomandibular joint, condyle, glenoid fossa, or oral or maxillofacial bone or tissue.

Bone remodeling can include changes in any bone characteristic, such as, but not limited to, bone shape, bone volume, bone density, or bone mineral content. In some embodiments, bone remodeling can include bone growth or resorption. Adjusting bone growth or bone resorption can result in altering bone shape or position (i.e., tooth movement). Increasing the quality or degree of bone remodeling can aid in adjusting the shape or position of bone (such as a mandibular bone or maxillary bone), or can aid in increasing the retention of teeth in a particular position, for example, in a position resulting from orthodontic treatment or resulting from oral or maxillofacial bone remodeling. Increasing the quality or degree of bone remodeling can aid in decreasing the potential for teeth to move back to a previous position, for example, a position prior to orthodontic treatment or prior to oral or maxillofacial bone remodeling. Thus, administration with light having a wavelength in the range of about 585 nm to about 665 nm, or about 605 nm to about 645 nm, or about 615 nm to about 635 nm, or about 625 nm, optionally also with light in the range of 815 nm to 895 nm, can be useful for stabilizing the movement of teeth prior to, subsequent to or concurrently with bone remodeling or orthodontic treatment.

Accordingly, in other embodiments, the present methods further comprise performing orthodontic treatment, such as installing one or more orthodontic appliances on the patient, prior to, subsequent to or concurrently with the administration of light. In one embodiment, the orthodontic appliance is a retainer device or a passive orthodontic appliance. Other suitable appliances can include, for example, removable retainers, such as a Hawley retainer, or a vacuum formed retainer, or fixed retainers, such as a bonded lingual retainer. These appliances can assist in maintaining tooth position prior to, subsequent to or concurrently with the administration of light, for example by stimulating bone remodeling. In some embodiments, the present methods further comprise regulating oral or maxillofacial bone remodeling, such as installing one or more functional appliances to a patient prior to, subsequent to or concurrently with the administration of light. Administration with light having a wavelength in the range of about 815 nm to about 895 nm, or about 835 nm to about 875 nm, or about 845 nm to about 865 nm, or about 855 nm, can also be useful for stabilizing tooth movement, in one embodiment prior to, subsequent to or concurrently with oral or maxillofacial bone remodeling or orthodontic treatment. In one embodiment, administration of light having wavelengths in the range of about 585 nm to about 665 nm increases bone remodeling to a greater degree or extent that does administration of light having wavelengths in the range of about 815 nm to about 895 nm.

Tooth-root resorption can include breakdown or destruction, or subsequent loss, of the root structure of a tooth. Tooth-root resorption can be caused by differentiation of macrophages into osteoclasts in surrounding tissue which, if in close proximity to the root surface can resorb the root surface cementum and underlying root dentine. Tooth-root resorption can be exacerbated by heavy or supra-physiologic orthodontic forces that exert on periodontal tissue pressure that is higher than the normal physiologic capillary and interstitial pressure. This prevents normal blood flow, which can cause schema (lack of blood supply) and ultimately cell death of soft tissue and bone in the periodontium. These dead tissues, otherwise known as a “hyalinized zone,” are removed through multi-nucleated cells and undermining respiration process and in many cases healthy bone, cementum and dentin are resorbed through this process.

Accordingly, administering light having a particular wavelength, is useful for modulating the speed, quality and type of bone remodeling, such as tooth movement, e.g., bodily or tipped, and for increasing or stabilizing tooth movement. In some embodiments, stabilizing tooth movement can comprise moving one or more teeth with less tipping. Stabilizing tooth movement can also comprise retarding or arresting tooth movements in particular ways. For example, this can comprise minimizing the amount of, or eliminating, slanting (or tipping). Administration of light can also be useful for inducing bone remodeling. Administration of light can also be useful for reducing, minimizing, or preventing tooth root resorption, bone resorption, inflammatory dentin or cementum resorption, or inflammation of tissue.

In some embodiments, the light is administered to substantially the entirety of a patient's body. In some embodiments, the light can be administered to substantially the entirety of a patient's oral or maxillofacial bone, muscle, or soft tissue, or one or more teeth, such as the patient's maxillary and mandibular bone. Alternatively, using a light-therapy apparatus or other suitable apparatus, light of one or more particular wavelengths can be administered to different selected regions of a patient's maxillary and mandibular alveolar bone, or teeth, in order to effect movement of the mandibular bone or maxillary bone or teeth (e.g., anchor (no movement), bodily, or tipped) in one or more regions of a patient's mouth. For example, one or more regions in which it is desired that the maxillary bone or mandibular bone or teeth not be moved or changed, or that the teeth serve as an anchor to facilitate movement of teeth in other selected regions of the patient's jaw, can be optionally screened or masked such that they receive no light. Alternatively, in one or more regions in which it is desired that the maxillary bone or mandibular bone or teeth not be moved or changed do not receive light as light emitters over such regions are turned off. Regions in which it is desired that bone remodeling occur or that teeth be moved bodily can be administered with light having a wavelength in the range of about 585 nm to about 665 nm, in the range of about 605 nm to about 645 nm, about 615 nm to about 635 nm, or about 625 nm. Regions in which it is desired to have bone remodeling or increase tooth movement but permit some tipping of the teeth can be administered with light having a wavelength in the range of about 815 nm to about 895 nm, about 835 nm to about 875 nm, about 845 nm to about 865 nm, or about 855 nm. Bone remodeling or tooth movement can be selectively regulated by administering an effective amount of light having one wavelength to one or more selected regions of a patient's maxillary bone, mandibular bone, temporal bone, and by administering an effective amount of light having a different wavelength to one or more different selected regions of the bone.

In some embodiments, light is administered within a narrow range of wavelengths (e.g., 50 nm or less, 30 nm or less, 20 nm or less, 10 nm or less, 5 nm or less), or at a single wavelength. In some embodiments, light is administered at a limited wavelength range (e.g., 1000 nm or less, 700 nm or less, 600 nm or less, 500 nm or less, 400 nm or less, 300 nm or less, 250 nm or less, 200 nm or less, 150 nm or less, 100 nm or less, or 75 nm or less). In some embodiments, the light administered does not include wavelengths beyond the narrow or limited range of wavelengths. The narrow or limited range of wavelengths can have any of the upper or lower limits of wavelength as described previously. In some embodiments, however, the light administered does not include light with a sufficient intensity to constitute an effective amount having wavelengths beyond the narrow or limited range of wavelengths.

In some embodiments, light is emitted at one, two, or more peak wavelengths of emission. A peak wavelength is the wavelength at which the highest intensity of light is emitted. In some embodiments, light is administered at a range of wavelengths that includes a peak wavelength having the highest intensity within the range. In some embodiments, a peak wavelength is at about 620 nm, about 640 nm, about 650 nm, about 655 nm, about 660 nm, about 665 nm, about 670 nm, about 680 nm, about 690 nm, about 800 nm, about 820 nm, about 830 nm, about 835 nm, about 840 nm, about 845 nm, about 850 nm, about 860 nm, about 870 nm, about 890 nm, about 910 or about 930 nm. In some embodiments, the administered light does not have wavelengths that vary from the peak wavelength by more than about 1 nm, about 2 nm, about 3 nm, about 5 nm, about 10 nm, about 15 nm, about 20 nm, about 30 nm, about 40 nm, about 50 nm, about 75 nm, about 100 nm, about 150 nm, about 200 nm, about 250 nm, about 300 nm, about 400 nm, or about 500 nm.

Where two or more light wavelengths are administered, the light can be administered at any ratio of each wavelength's intensity. For example, light administered at a first wavelength can have an intensity that is about 1.1×, 1.2×, 1.3×, 1.5×, 1.7×, 2.0×, 2.5×, 3.0×, 3.5×, 4.0×, 5.0×, 10×, 12×, 15×, 20×, 30×, 50×, 100× that of light administered at a second wavelength. In some embodiments, the administered light is emitted from one or more light emitters, in another embodiment, from one or more light emitters having a first set of properties and, optionally, from a second set of light emitters having a second set of properties. In other embodiments, the number of light emitters having a first set of characteristics exceeds that of the light emitters having a second set of characteristics. For example, the number of light emitters having the first set of characteristics can be about 1.1×, 1.2×, 1.3×, 1.5×, 1.7×, 2.0×, 2.5×, 3.0×, 3.5×, 4.0×, 5.0×, 10×, 12×, 15×, 20×, 30×, 50×, 100× the number of light emitters having the second set of characteristics, or vice versa.

The light can optionally be substantially monochrome. When light is “substantially monochrome” it consists of a single wavelength or comprises other wavelengths that are emitted at an intensity that is ineffective in the present methods, including for regulating oral or maxillofacial bone remodeling when administered to the oral or maxillofacial bone, muscle, or soft tissue, or one or more teeth of a patient, with or without allowing a functional appliance to exert a force on oral or maxillofacial bone, muscle, or soft tissue, or one or more teeth of the patient. In some embodiments, a substantially monochromatic light is emitted at a narrow range of wavelengths without being emitted at other wavelengths outside the range or without an effective intensity of light being emitted at other wavelengths outside the range. In some embodiments, a substantially monochromatic light is emitted within an about 5 nm or less, about 10 nm or less, or about 20 nm or less wavelength range without being emitted at other wavelengths outside the range or without an effective intensity of light being emitted at other wavelengths outside the range. Administering light from light emitters that emit at multiple wavelengths can allow for irradiation over multiple wavelengths or greater selectivity and precision in administration. The light can optionally comprise incoherent light. In some embodiments, light is administered at a single frequency, light can have a phase that drifts relatively quickly, a pulse of light waves can have an amplitude that changes quickly, or a light wave can encompass a broad range of frequencies.

Light can be administered directly from a light emitter to a predetermined area. Light can be emitted and can travel directly through a patient's skin, such as the patient's face, to a region. In another embodiment, the light is administered intra-orally to a region of the oral tissue (e.g., alveolar mucosa). In some embodiments, light is modified by optics before reaching the patient's face or traveling through the patient's skin. For example, light can be diffused, focused, parallel, reflected, redirected, or filtered after it is emitted and before it reaches the patient's face or travels through the patient's skin. In one embodiment, light of one or more wavelengths is selectively blocked or partially filtered before reaching the patient's face or a region. In some embodiments, light diverges or converges from an emission source before reaching the region. For example, light can diverge in a beam having an included angle θ in the range of about 45-60°. The emitted light diverge to have an included angle θ of 0 to about 15°, 0 to about 30°, 0 to about 45°, 0 to about 60°, 0 to about 75°, 0 to about 90°, or 0 to about 120°.

Light that irradiates the region can optionally have the same or about the same characteristics as light that is emitted. In some embodiments, light that reaches the region does not have the same characteristics as the light that is emitted. One or more of the light characteristics can optionally be altered prior to administration or when it passes through the face of the patient. One or more of the light characteristics can optionally be altered when it passes through optics, such as one or more lenses or mirrors. For example, one or more of the light characteristics can be altered in the range of about ±20% or less, about ±15% or less, about ±10% or less, about ±5% or less, about ±3% or less, about ±1% or less, about ±0.5% or less, or about ±0.1% or less.

An effective dosage of light can have an energy density that irradiates from a light source. For example, an effective dosage of irradiated light can be from about 24 J/cm² to about 200 J/cm². The effective dosage of irradiated light can be administered once or repetitively. In some other embodiments, the effective dosage has an irradiated light energy density that is from about 30 J/cm² to about 100 J/cm². In other embodiments, the dosage of light is about 5 J/cm² or less, about 10 J/cm² or less, about 20 J/cm² or less, about 30 J/cm² or less, about 50 J/cm² or less, about 75 J/cm² or less, about 100 J/cm² or less, about 125 J/cm² or less, about 150 J/cm² or less, about 175 J/cm² or less, or about 200 J/cm² or less. The dosage of light can be about 1 J/cm² or more, about 5 J/cm² or more, about 10 J/cm² or more, about 20 J/cm² or more, about 25 J/cm² or more, about 30 J/cm² or more, about 40 J/cm² or more, about 50 J/cm² or more, about 60 J/cm² or more, about 75 J/cm² or more, about 100 J/cm² or less, about 125 J/cm² or more, about 150 J/cm² or more, or about 175 J/cm² or more. The dosage of irradiated light can be in a range bounded by any of the energy density values described herein. The dosage of light can be increased, for example, by using a light source that emits light having a relatively higher average intensity, or by increasing the duration of administration of light.

An effective dosage of light can have an energy density that reaches a region, such as the mandibular bone, maxillary bone, or temporal bone. For example, an effective dosage of light that reaches a region can be from about 0.5 J/cm² to about 100 J/cm². The effective dosage of light that reaches the region can be administered once or repetitively. In some other embodiments, the effective dosage has an irradiated light energy density that is from about 1 J/cm² to about 50 J/cm². In other embodiments, the dosage of light is about 0.5 J/cm² or less, about 1 J/cm² or less, about 2 J/cm² or less, about 5 J/cm² or less, about 10 J/cm² or less, about 15 J/cm² or less, about 20 J/cm² or less, about 30 J/cm² or less, about 40 J/cm² or less, about 50 J/cm² or less, about 70 J/cm² or less, about 80 J/cm² or less, about 90 J/cm² or less, or about 100 J/cm² or less. The dosage of light can be about 0.5 J/cm² or more, about 1 J/cm² or more, about 2 J/cm² or more, about 3 J/cm² or more, about 5 J/cm² or more, about 10 J/cm² or more, about 15 J/cm² or more, about 20 J/cm² or more, about 30 J/cm² or more, about 40 J/cm² or more, about 50 J/cm² or less, about 60 J/cm² or more, about 70 J/cm² or more, or about 80 J/cm² or more. The dosage of light that reaches the region can be in a range bounded by any of the energy density values described herein.

The duration over which the effective dosage, which is optionally repetitive, is administered can range from about 10 minutes to about 40 minutes. In some embodiments, the dosage is administered over a period of time equaling about 30 seconds or more, about 1 minute or more, about 2 minutes or more, about 3 minutes or more, about 5 minutes or more, about 7 minutes or more, about 10 minutes or more, about 15 minutes or more, about 20 minutes or more, about 25 minutes or more, about 30 minutes or more, about 40 minutes or more, about 50 minutes or more, about 1 hour or more, about 1 hour 15 minutes or more, about 1 hour 30 minutes or more, or about 2 hours or more. In other embodiments, the dosage is administered over a period of time equaling about 3 minutes or less, about 5 minutes or less, about 10 minutes or less, about 15 minutes or less, about 20 minutes or less, about 25 minutes or less, about 30 minutes or less, about 35 minutes or less, about 40 minutes or less, about 50 minutes or less, about 1 hour or less, about 1 hour 15 minutes or less, about 1 hour 30 minutes or less, about 2 hours or less, or about 4 hours or less. Alternatively, the dosage can be administered in a range of time within any of the time values described herein. Such light therapy can include light emission that has been provided externally, such as, for example, extra-orally. In some embodiments, one or more internal, such as, for example, intra-oral, light blocking masks or shades can be used. An internal or oral mask can block one or more wavelengths of light, or can reduce the intensity of one or more wavelengths of light, from reaching a region covered by the internal or oral mask. This can include an upper arch (e.g., maxillary teeth), lower arch (e.g., mandibulary teeth), right side of the mandibular bone, left side of the mandibular bone, right side of the maxillary bone, left side of the maxillary bone, right side of the temporal bone, or the left side of the temporal bone, such as the right temporomandibular joint, left temporomandibular joint, right condyle, left condyle, right glenoid fossa, or left glenoid fossa. A mask can be provided for any oral or maxillofacial bone, muscle, or soft tissue, or one or more teeth. Accordingly in other embodiments the methods further comprise applying an intra-oral or extra-oral shade or mask to the patient. The intra-oral or extra-oral shade or mask can be applied prior to or concurrently with the administration of light.

Any time period (or duration) can be provided between dosages. For example, the time period between dosages can be on the order of seconds, minutes, hours, days, weeks, months, quarter of a year, or years.

The effective dosage, which in some embodiments is repetitive, can be administered with any desired frequency, e.g., four times daily, three times daily, twice daily, daily, every second day, weekly, biweekly, monthly, or quarterly. In some embodiments, dosage is administered at regular intervals (e.g., daily), while in other embodiments, the dosage is not administered at regular intervals (e.g., administration can occur 2 times a week at any time during the week). In one embodiment, light is administered in the morning and at night. Light can be administered throughout the time period that a patient is undergoing bone remodeling or tooth movement. In some embodiments, a patient undergoes orthodontic treatment in addition to undergoing bone remodeling or tooth movement. Orthodontic treatment can occur prior to, subsequent to, or concurrently with oral or maxillofacial bone remodeling. Light can be administered throughout the time period that a patient is undergoing orthodontic treatment, or following treatment to stabilize tooth movement. For example, light can be administered after a functional appliance or an orthodontic appliance is applied, removed, adjusted, after an appointment, or after an active phase, as described herein. It can be desirable to administer light with greater frequency, e.g. four times daily, three times daily, twice daily, daily or every second day, while a patient is undergoing orthodontic treatment. Where light is being administered for oral or maxillofacial bone remodeling, for stabilizing tooth movement or to reduce tooth-root resorption, treatments of reduced frequency, e.g. weekly, biweekly, monthly, or quarterly, can be used to minimize inconvenience to patients.

Light can be administered for any length of time. In some embodiments, light is administered on the order of weeks, months, quarters, or years. For example, light can be administered while an orthodontic appliance, such as a functional appliance, exerts a force on one or more teeth. One or more dosages of light can be administered over a period of time during which a patient is undergoing oral or maxillofacial bone remodeling during which a functional appliance exerts a force on one or more teeth. In some embodiments, one or more dosages of light can be administered over a period of time during which a force is exerted on one or more teeth, during which a patient is wearing an orthodontic appliance that itself can exert a force, such as a heavy force, or during which a patient is undergoing orthodontic treatment during which a force, such as a heavy force, can be applied. In some embodiments, while a patient is undergoing orthodontic treatment or is wearing an orthodontic appliance, a patient is administered with light. Administration of light, which can include regular, irregular, continuous or discontinuous administration of light, can be on the order of days, weeks, months, quarters, or years. In some embodiments, light is administered over a plurality of days, weeks, months, quarters, or years. In some embodiments, light is administered over a plurality of sessions. In some embodiments, one or more hours, days, weeks, months, quarters, or years occur between sessions.

If the light emitters are pulsed, then their duty cycle can be adjusted as desired; e.g., light can be administered with a duty cycle of about 10%, about 20%, about 30%, about 40%, about 50%, about 60%, about 70%, about 80%, or about 90%. The pulsing can occur with any frequency. For example, light can be pulsed every picosecond, nanosecond, microsecond, millisecond, second, multiple seconds, or minutes. Frequencies can include, but are not limited to, about 1 mHz, about 10 mHz, about 50 mHz, about 100 mHz, about 500 mHz, about 1 Hz, about 2 Hz, about 5 Hz, about 10 Hz, about 15 Hz, about 20 Hz, about 25 Hz, about 30 Hz, about 35 Hz, about 40 Hz, about 50 Hz, about 70 Hz, about 100 Hz, about 200 Hz, about 500 Hz, or about 1 kHz. Any of the aforementioned characteristics of light emission (e.g., whether the light is on or off, continuous or pulsed, duty cycle, frequency, intensity, wavelength) can be varied or maintained. Where the light is emitted from a source having a controller, any characteristics of light emission can be varied or maintained in accordance with instructions from its controller.

Where the light is emitted from one or more lights, light can be controlled so that the number of lights that are on or off at a given period can be individually controllable. For example, in some embodiments, a light source is turned on or off relative to other light sources. Various light sources can be modulated individually (e.g., one or more properties of a particular light source can be varied) or otherwise individually controlled, to expose individual sections of a patient to a desired energy density. In some embodiments, light sources can be modulated individually, to expose individual sections of a patient's bone or other regions to a desired energy density. In some embodiments, light sources can be modulated individually, to expose individual sections of a patient's oral or maxillofacial bone, muscle, or soft tissue, or one or more teeth, such as a maxillary bone, mandibular bone, temporal bone, or other regions to a desired energy density. This can be desirable when it is desirable to administer light to different regions. Thus, the position of light being administered can be varied. In another embodiment, different types of light sources are turned on or off relative to other light emitters. For example, at some times, light emitted in a first wavelength range can be turned on while light emitted in a second wavelength range can be turned off, vice versa, or both types of light emitters can be turned on or off. Thus, the wavelength of light being administered can be varied. In some embodiments, the intensity of light being administered is varied (e.g., by turning some light sources on or off, or varying the intensity emitted by the light sources). Administering light selectively can enable an increased anchorage effect (by reason of lower tooth mobility) of teeth which are not exposed to any light, which can thereby permit for more precise bone remodeling or movement of teeth during which light is administered. In some embodiments, the soft tissue is alveolar mucosa.

In some embodiments, where infrared light is administered to a region, a visible light is also emitted. In one embodiment, the visible light is bright, e.g., uncomfortable for a patient to look at. The bright visible light can deter users or patients from looking into a light source when it is operating, can provide a perceptible indication that a light is being emitted, and can be useful in properly positioning a light source. The visible light can be, but is not necessarily, of a wavelength range that is beneficial in the present methods, including for light therapy or regulating oral or maxillofacial bone remodeling. In some embodiments, the ratio of the intensities of the visible and infrared components of the light is 1 part or less visible light to 5 parts or more infrared light. In other embodiments, the ratio of the intensities of visible and infrared components is about 1 part or more visible light to 5 parts or more infrared light, 1 part or more visible light to 3 parts infrared light, 1 part or more visible light to 2 parts infrared light, 1 part or more visible light to 1 part infra red light, 2 parts or more visible light to 1 part infrared light, 3 parts or more visible light to 1 part infrared light, 5 parts or more visible light to 1 part infrared light, 10 parts or more visible light to 1 part infrared light, or substantially no infrared light. In some embodiments, light is emitted within a range that includes wavelengths less than an order of magnitude relative to one another. Alternatively, the range can include wavelengths emitted at one, two, three or more orders of magnitude relative to one another.

The region and desired light characteristics can vary from patient to patient. A physician, dentist, other health-care provider or patient can determine a light treatment regimen for a patient.

In some instances, it is desirable to administer light to less than all regions of the patient's bone. For example, in some instances, it can be desirable to administer light to less than all regions of the patient's maxillary or mandibular bone, for example, if it is desired that teeth or other regions should not be moved (e.g. it can be desired to regulate the movement of only the mandibular bone of a patient, or only the maxillary bone, or to use certain teeth as an anchor when regulating the movement of other teeth by not administering light to, e.g., blocking light from, the anchor teeth). Administering light to selected regions of the patient's oral or maxillofacial bone, muscle, or soft tissue, or muscle, or one or more teeth can comprise causing light to irradiate one or more selected regions of the patient's oral or maxillofacial bone, muscle, or soft tissue, or one or more teeth, such as tooth roots through the bone.

In one embodiment, light is selectively administered to less than all regions of the patient's mouth before, during, or after the exertion of a force. In one embodiment, light is not administered to an anchor region or tooth. In this embodiment, an orthodontic appliance, such as a functional appliance, is located between the anchor region or tooth and one or more selected bone region sought to be remodeled. The orthodontic appliance can exert a force on the selected bone region, for example, on another tooth. In some embodiments, the force is a heavy force. In some embodiments, an effective amount of light is administered to the selected bone region or other tooth and not to the anchor region or anchored tooth. The administration of light can increase the rate of the selected bone remodeling region or velocity (or rate of movement) of the other tooth and reduce, minimize, or prevent root resorption of the other tooth, while not increasing the rate of bone remodeling of the non-selected regions or velocity of the anchor tooth.

It can also be desirable to administer light of different wavelengths to different regions of the patient's oral or maxillofacial bone, such as a maxillary bone, mandibular bone, or temporal bone or teeth, if it is desired to differentially manipulate the movement or remodeling of the patient's teeth or oral or maxillofacial bone, such as the maxillary bone, mandibular bone, or temporal bone. In some embodiments, the right and left sides of a patient's mandible are not in need of the same about of remodelling. For example, the right side of the patient's mandible might need to be remodeled and lengthened by 5 mm whereas the left side might need to be remodeled and lengthened by 2 mm. To shorten the period of time required for the mandible to become symmetric, the right side of the mandibular can receive light treatment while the left side does not. In another example, light of a first wavelength can be administered to a first region and light of a second wavelength can be administered to a second region. The first and second wavelengths can include any wavelengths described elsewhere herein, such as about 585 nm to about 665 nm, about 666 nm to about 814 nm or about 815 nm to about 895 nm.

Light can be administered over an area (also referred to herein as a “light irradiation area”). For example, in some embodiments, light is administered to a region with an area. In some such embodiments, light characteristics remain uniform over the area. For example, light intensity can be uniform over the area. In other embodiments, however, light characteristics vary over the area. For example, light intensity can vary over the area.

Light can be administered to a light irradiation area of any size and shape. For example, a region, such as a specified region of the patient's maxillary bone, mandibular bone, or temporal bone can have any size or shape. The light irradiation area can have one or more dimensions (e.g., length, width, diameter) that range from about 1 to about 80 mm, or from about 1 to about 70 mm. In some embodiments, the light irradiation area has one or more dimensions (e.g., length, width, diameter) that range from about 1 to about 3 mm, about 3 to about 5 mm, about 5 to about 7 mm, about 7 to about 10 mm, about 10 to about 15 mm, about 15 to about 20 mm, about 20 to about 25 mm, about 25 to about 30 mm, about 30 to about 35 mm, about 35 to about 40 mm, about 40 to about 50 mm, about 50 to about 60 mm, or about 60 to about 80 mm.

A light irradiation area can have any shape, which can include, but is not limited to, a substantially rectangular shape, square shape, triangular shape, hexagonal shape, octagonal shape, trapezoidal shape, circular shape, elliptical shape, crescent shape, cylindrical shape or half-circle. In some embodiments, the dimensions of a light source is about the same as dimensions for a light irradiation area. In other embodiments, the dimensions of a light source is greater than the dimensions of a light irradiation area. Alternatively, the dimensions of a light source can be less than the dimensions of the light irradiation area. The relative areas of a light source and light irradiation area can depend on any angle, which can be a parallel, convergence, or divergence angle, at which light is emitted.

In some embodiments, an effective dosage of light is provided in a treatment regimen. The treatment regimen can be used in the present methods, including a method to regulate oral or maxillofacial bone remodeling or tooth movement through the administration of an effective dosage of light. The treatment regimen can also be used in a method for adjusting the position of a mandibular or maxillary bone, lengthening or shortening a mandibular bone, or any other form of oral or maxillofacial bone remodeling. In some embodiments, treatment regimens regulate bone remodeling of a mandibular or maxillary bone, or more tooth, upon which forces are or were exerted. The treatment regimen can also be used in a method for remodeling maxillary or mandibular bone. The treatment regimen can further be used in a method for reducing tooth-root resorption. In other embodiments, a treatment regimen can be provided for preventing or minimizing tooth-root resorption. Treatment regimens are provided for methods for reducing, preventing or minimizing bone resorption or inflammatory dentin or cementum resorption of the tooth root or periodontium. In some embodiments, treatment regimens are useful for reducing, preventing or minimizing inflammation of tissue surrounding one or more teeth upon which forces are or were exerted.

In one embodiment, a typical treatment regimen provides a dose of light daily. Each of the daily doses of light can be administered over a period lasting from a few minutes to about an hour. For example, daily ½ hour doses of light can be effective and are not unduly inconvenient for patients. A single daily dose can be as effective as dividing the same dose into multiple sessions administered at different times during the day. Some treatment regimens can comprise administering light in 5 treatments per week for 12 weeks. Each treatment can last ½ hour and irradiate the tissues of a patient's jaw with light having wavelengths of 660 nm and 840 nm. The 660 nm light can have an intensity of about 20 mW/cm² at the skin's surface. The 840 nm light can have an intensity of about 10 mW/cm² at the skin's surface. These treatment regimens can enhance bone density.

Other treatment regimens can comprise administering light in daily treatments for 21 days. Each treatment lasts between 20 minutes and one hour and irradiates the tissues of a patient's jaw with light having a wavelength of 618 nm and an intensity of 20 mW/cm² at the skin's surface. These treatment regimens can accelerate healing of bone grafts.

Another treatment regimen can comprise a twice-daily administration of light for six months. In one embodiment the light is administered from a light-therapy apparatus. Light can be administered at a wavelength of about 660 nm or about 840 nm, or at both wavelengths. The intensity of the light can be about 20 mW/cm² at the skin's surface. A functional appliance can be present in the patient's mouth while the light is administered. Subsequent to the first 6 month period, a second 6 month period can be provided where light is administered once every other day. The same functional appliance or one or more orthodontic appliances can be present in the patient's mouth at this time. The administration of light can optionally become less frequent or be administered at a lower intensity as treatment progresses.

Another treatment regimen can comprise administering light to a tooth having an orthodontic appliance and subsequently adjusting the orthodontic appliance. An orthodontic appliance can be installed on the patient's teeth prior to, subsequent to, or concurrently with the installation of a functional appliance. In some embodiments, adjusting an orthodontic appliance increases or alters the magnitude of a force exerted on one or more teeth. Adjusting an orthodontic appliance can alter the direction of a force exerted on one or more teeth. Light can be administered to one or more selected teeth for up to an hour prior to adjusting an orthodontic appliance. Adjusting the orthodontic appliance can cause a force to be exerted on the one or more teeth. Adjusting the orthodontic appliance can change the magnitude or direction, or both, of the force exerted. Adjusting the orthodontic appliance can comprise tightening, loosening or replacing one or more of the appliance's wires, springs or clastic devices. Different sizes, materials, or shapes of such components can be used. Light can then be administered daily to the one or more selected teeth, until the next adjustment of the orthodontic appliance. This administration of light can reduce, minimize, or prevent tooth-root resorption, bone resorption, tissue inflammation, periodontium resorption or cementum resorption.

Another treatment regimen can comprise administering vitamin D to a patient, administering light to a region of the mandibular bone, maxillary bone, or temporal bone such as a temporomandibular joint, condyle, or glenoid fossa, having a functional appliance and subsequently adjusting an orthodontic appliance, such as a functional appliance. In some embodiments, adjusting a functional appliance (or any other orthodontic appliance) increases or decreases the magnitude of a force exerted on one or more teeth, mandibular bone, maxillary bone, or temporal bone. Adjusting a functional appliance also can alter the direction of a force exerted. Light can be administered to one or more selected regions for up to an hour prior to adjusting a functional appliance (or any other orthodontic appliance). Adjusting the functional appliance (or any other orthodontic appliance) can cause a force to be exerted on the one or more teeth, mandibular bone, maxillary bone, or temporal bone. Adjusting the functional appliance (or any other orthodontic appliance) can change the magnitude or direction, or both, of the force exerted. Adjusting the functional appliance (or any other orthodontic appliance) can comprise tightening, loosening or replacing one or more of the appliance's wires, springs or elastic devices. Different sizes, materials, or shapes of such components can be used. Light can then be administered daily to the one or more selected region, until the next adjustment of the functional appliance. This administration of light can regulate oral or maxillofacial bone remodeling. In some embodiments, the administration of light regulates tooth movement. For example, the administration of vitamin D and administration of light can increase the rate of bone remodeling or tooth movement. This can decrease the amount of time that a functional appliance (or any other orthodontic appliance) is worn or needs to be worn by a patient.

The present methods can further comprise controlling temperature of the patient's skin (such as the patient's face) or of any light source that contacts or is close to a patient's skin or a region. For example, the method can comprise cooling, heating, or maintaining the temperature at a patient's face. A patient's face can be contacted with a temperature control mechanism, which can cause the removal or provision of heat. In some embodiments, heat can be generated by the light source. In some embodiments, the temperature of the light source can be controlled. A temperature control mechanism can communicate with the light source. Heat can be removed from or provided to the light source. Any embodiments for temperature regulation described herein can be used within the method. The method can further comprise measuring a temperature at a patient's face or at a light source. Temperature regulation can optionally occur in response to one or more temperature measurements made.

In one embodiment, regulating bone remodeling occurs prior to, subsequent to or concurrently with orthodontic treatment useful for regulating tooth movement of a patient. In one embodiment, the administration of light is repetitive.

Oral or maxillofacial bone remodeling can occur at the mandibular bone, maxillary bone, or temporal bone. In some embodiments, oral or maxillofacial bone remodeling can occur at a joint, such as the temporomandibular joint. The some embodiments, oral or maxillofacial bone remodeling can occur at a condyle or glenoid fossa. The regulation of oral or maxillofacial bone remodeling can result in the repositioning of the mandibular bone or maxillary bone, the lengthening or shortening of the mandibular bone or maxillary bone, or altering the angle, shape, or dimensions of the mandibular bone or maxillary bone.

Oral or maxillofacial bone remodeling can comprise installing a functional appliance in a patient. A functional appliance can be present on one or more teeth of a patient. The methods can comprise installing a functional appliance in a patient, such as installing the appliance on one or more teeth, the patient's gums, the patient's maxillary or mandibular bone, or other oral or maxillofacial features of the patient, adjusting a functional appliance of the patient, or can comprise removing a functional appliance from the patient. A treatment for oral or maxillofacial bone remodeling can include a period of time during which the functional appliance is installed in the patient. In some embodiments, treatment for oral or maxillofacial bone remodeling can include a period of time after the functional appliance has been installed in or removed from the patient. In some embodiments, treatment for oral or maxillofacial bone remodeling can include a period of time preceding the installation of a functional appliance. In other embodiments treatment for oral or maxillofacial bone remodeling includes a period of time prior to, during, or subsequent to the exertion of a force on oral or maxillofacial bone, muscle, soft tissue, or one or more teeth, such as mandibular bone, maxillary bone, temporal bone, or on one or more oral muscles that can prevent the oral muscles from exerting a force on the one or more teeth, mandibular bone, maxillary bone, temporal bone. Treatment for oral or maxillofacial bone remodeling can include a period of time while a patient is seeing or consulting with an orthodontist or other dental specialist.

Treatment for oral or maxillofacial bone remodeling, including methods for regulating such remodeling, can comprise an active stage and a passive stage. An active stage can comprise some time during which a functional appliance is installed in and/or on the patient. In some embodiments, an active stage includes a time during which a force is exerted on a tooth, mandibular bone, maxillary bone, temporal bone. An active stage can include a period during which the patient is undergoing one or more adjustments to the patient's functional appliance. A passive stage can include a period after a functional appliance has been removed from the patient. In some embodiments, a passive stage includes a period during which a functional appliance is installed, but is no longer undergoing adjustments. In some embodiments, a passive stage includes a period during which there is no further muscular tension on the jaw or teeth when the functional appliance is in position, which typically occurs after a period of treatment and bone remodeling. In some embodiments, a passive stage includes a period during which a functional appliance is not providing force to effect bone remodeling. Instead, the passive stage can include a period during which a functional appliance is installed in a patient and that maintains the maxillary bone or mandibular bone in its position. Any stage of oral or maxillofacial bone remodeling can last on the order of days, weeks, months, quarters, or years.

In some embodiments, an orthodontic treatment is provided prior to, subsequent to, or concurrently with a treatment for oral or maxillofacial bone remodeling. An orthodontic treatment can cause one or more teeth to move or maintain its position relative to a supporting maxillary bone or mandibular bone, or can include regulation of tooth movement. In some embodiments, orthodontic treatment comprises aligning teeth. Orthodontic treatment can comprise treating malocclusion, which can occur when teeth fit together improperly, for example, as a result of their individual positions or positions of underlying jaw bone as they relate to one another. Malocclusion can be treated using light therapy or tooth movement regulation according to the methods described herein. Accordingly, the present invention further relates to methods for treating or preventing malocclusion, comprising extra-orally administering to a patient in need thereof an effective amount of light transdermally to a region of the patient's maxillary or mandibular alveolar bone. In another embodiment, methods for treating or preventing malocclusion comprise intra-orally administering to a patient in need thereof an effective amount of light to a region of the patient's maxillary or mandibular alveolar bone. The methods for treating or preventing malocclusion can further comprise administering an effective amount of vitamin D.

An orthodontic treatment can comprise removably coupling an orthodontic appliance to one or more teeth of a patient. In some embodiments, orthodontic treatment can occur via a functional appliance which can result in tooth movement while regulating oral or maxillofacial bone remodeling. An orthodontic appliance can be present on, or removably coupled to, one or more teeth of a patient. The methods can comprise installing an orthodontic appliance to a patient, such as installing the orthodontic appliance to one or more teeth of the patient, adjusting an orthodontic appliance of the patient, or can comprise removing an orthodontic appliance from the patient. In some embodiments, an orthodontic appliance can be installed or removed prior to, subsequent to, or concurrently with the installation or removal of a functional appliance. Orthodontic treatment can include a period of time during which the orthodontic appliance is installed in the patient. In some embodiments, orthodontic treatment can include a period of time after the orthodontic appliance has been installed in or removed from the patient. In some embodiments, orthodontic treatment can include a period of time preceding the application of an orthodontic appliance. In other embodiments orthodontic treatment includes a period of time prior to, during, or subsequent to the exertion of a force on one or more teeth. Orthodontic treatment can include a period of time while a patient is seeing or consulting with an orthodontist.

In some embodiments, orthodontic treatment includes an active stage and a passive stage. An active stage can include some time during which an orthodontic appliance is installed in the patient. In some embodiments, an active stage can include a time during which a force is exerted on a tooth to effect tooth movement. In some embodiments, the force exerted on a tooth during an active stage is a heavy force. An active stage can include a period during which the patient is undergoing one or more adjustments to the patient's orthodontic appliance. A passive stage can include a period after an appliance has been removed from the patient. In some embodiments, a passive stage can include a period during which an appliance is installed but is no longer undergoing adjustments. In some embodiments, a passive stage can include a period during which an orthodontic appliance no longer exerts a force on the teeth. In some embodiments, a passive stage can include a period during which an orthodontic appliance is not providing force to effect movement of a tooth. Instead, the passive stage can include a period during which an orthodontic appliance is installed in a patient and that maintains one or more teeth in its position. In some embodiments, any stage of orthodontic treatment can last on the order of days, weeks, months, quarters, or years.

In some embodiments, orthodontic treatment and bone remodeling or tooth movement occurs concurrently. In some embodiments, oral or maxillofacial bone remodeling results in tooth movement. Force can be exerted on one or more tooth, any region of the jaw, or any other region of the mouth or head. Force can be exerted by a functional appliance or an orthodontic appliance. In some embodiments, the force is a heavy force. Bone remodeling can involve altering the position or morphology of bone, including the jaw bone. For example, a jaw bone can be moved forward, or can be lengthened. Other examples of bone remodeling, as described herein, can also be applicable. In some embodiments, oral or maxillofacial bone remodeling occurs in conjunction with regulating tooth movement. Accordingly, the present methods are useful for bone remodeling. Present methods can further comprise bone remodeling. Light can be administered to a region, such as a region of a jaw bone, or any other oral or maxillofacial bone, muscle, or soft tissue, and is useful for bone remodeling. Accordingly, the invention further provides methods for inducing bone remodeling, comprising extra-orally administering an effective amount of light transdermally or intra-orally to a region of oral tissue of a patient in need thereof. Light therapy can be provided in conjunction with oral or maxillofacial bone remodeling, and can increase the rate of oral or maxillofacial bone remodeling. For example, applying an effective amount of light as described in the present methods can reduce the amount of time to achieve the same degree of bone remodeling without light by about 10%, about 20%, about 30%, about 40%, about 50%, about 60%, about 70%, about 80%, or about 90%. Light treatment can promote bone remodeling which can increase the rate of bone remodeling of the maxillary or mandibular bone or tooth movement. This can allow heavier forces to be used, which could accelerate tooth movement even more than with lighter forces. Such forces can be exerted by one or more appliances.

The present methods can be performed on a patient prior to being applied with one or more functional appliances or orthodontic appliances, during a time when the patient wears one or more functional appliances or orthodontic, or after one or more functional appliances or orthodontic appliances has been removed from the patient. A functional appliance or orthodontic appliance can be fixed or movable with respect to a patient's teeth. Orthodontic appliances can include, for example, fixed active appliances such as pin and tube appliances, appliances using wires or brackets or springs, ribbon arch appliances, Begg lightwire appliances, edgewise appliances, pre-adjusted edgewise appliances, self-ligating edgewise appliances, hi-helix, tri-helix, quad-helix, rapid maxillary expansion appliance (RPE); removable active appliances such as expansion and labial segment alignment appliance INVISALIGN™; or orthodontic headgear including reverse headgear and conventional headgear; and other types of orthodontic apparatus.

In one embodiment, the orthodontic appliance is fixed. Examples of fixed orthodontic appliances include pin and tube appliances, ribbon arch appliances, Begg Lightwire appliances, edgewise appliances, pre-adjusted edgewise appliances, self-ligating edgewise appliances, hi-helix appliances, tri-helix appliances, quad helix appliances, rapid maxillary expansion appliances (RME), or pin stripe appliances. Fixed orthodontic appliances can be fixed to the patient's teeth during orthodontic treatment. In another embodiment, the orthodontic appliance is removable. Examples of removable orthodontic appliances include Active Hawley appliances, Invisalign aligners, aligners, fan expanders, or sagittal appliances.

In some embodiments, the functional appliance is a mandibular repositioner or any other intra-oral device that repositions the mandible to create tension on tissue to stimulate bone remodeling or tooth movement. Some examples of mandibular repositioners are Herbst, Twin Block, Fixed Twin Block, Bonded Twin Block, Biobloc, Forsus Fatigue (e.g., EZ2), Xbow (Crossbow), mandibular anterior repositioning appliance (Mara), Bass Dynamax, Bionator, Open Face Activator, Activator, Woodside Activator, Frankel (e.g., Frankel I, II, III, IV, V), Teuscher appliance, Andreson appliance, 3-Way Sagittal, Lower Schwartz, 3 Way Expander, jaw repositioning appliances, removable orthotic appliances, Series 2000®, BioPedic Appliance, Rick-A-Nator™, Ritto Appliance, Eureka Spring™, Twin Force Bite Corrector™, Alpern Class II Closers, Rapid palatal expander, Tandem™, facemask, reverse pull headgear, and conventional orthodontic headgear.

In one embodiment, the functional appliance is fixed. A fixed functional appliance can be cemented, for example, on one or more teeth. Some examples of fixed functional appliances include Herbst, Fixed Twin Block, Bonded Twin Block, Forsus Fatigue (e.g., EZ2), Xbow (Crossbow), Series 2000®, BioPedic Appliance, Rick-A-Nator™, Ritto Appliance, Eureka Spring™, Twin Force Bite Corrector™, Alpern Class II Closers, and Rapid palatal expander. In another embodiment, the functional appliance is removable. Some examples of removable functional appliances include Twin Block, Biobloc, mandibular anterior repositioning appliance (Mara), Bass Dynamax, Bionator, Open Face Activator, Activator, Woodside Activator, Frankel (e.g., Frankel I, II, III, IV, V), Teuscher appliance, Andreson appliance, 3-Way Sagittal, Lower Schwartz, 3 Way Expander, jaw repositioning appliances, and removable orthotic appliances. In some embodiments, the functional appliance is a combination fixed-removable functional appliance. A combination fixed-removable functional appliance can include one or more component that is fixed to a patient's teeth and one or more component that is removable from the fixed component. Some examples of combination fixed-removable functional appliances include Tandem™, a facemask, reverse pull headgear, and conventional orthodontic headgear.

In some embodiments, the functional appliance is a Class II corrector. Some examples of Class II correctors include Herbst, Twin Block, Forsus Fatigue, and Mara. In other embodiments, the functional appliance is a Class I corrector that is useful for creating and bony and dental expansion of crowded and lower arches. In other embodiments, the functional appliance is a Class III corrector that is useful for stimulating maxillary forward growth, or retruding or limiting mandibular growth.

In some embodiments, the functional appliances reposition a patient's mandibular bone anteriorly. The functional appliance can be a fixed functional mandibular repositioner. Examples of such functional appliances are a Herbst, Twin Block, Bonded Twin Block, Biobloc, and Bass Dynamax. Light can be administered to a temporomandibular joint, condyle, or glenoid fossas of temporal bone to remodel. In some embodiments, the functional appliances expand the jaw (e.g., using muscular pressure or lack of muscular forces to allow teeth to move and/or bone to remodel). Examples of such functional appliances can include Bionator, Open Face Activator, Activator, Woodside Activator, or Frankel. Light can be administered to alveolar bones and teeth, as these appliances can cause orthodontic movement of teeth as well as bone remodeling. In some embodiments, the functional appliances control growth of the maxillary bone or mandibular bone. Examples of such functional appliances can include a facemask, or reverse pull headgear. Light can be administered to apical areas of the jaw, which can cause some orthodontic movement, but primarily remodels and provides anterior movement of maxillary bone. In some embodiments, the functional appliances exert a force on, or cause bone remodeling at, a temporomandibular joint, condyle, or glenoid fossa of a patient.

In some embodiments, a functional appliance or an orthodontic appliance comprises steel wires, nickel titanium wires, or titanium molybdenum wires. In some embodiments, the functional appliance or orthodontic appliance comprises wires or springs that are of a high gauge. Some examples of wires that a functional appliance or orthodontic appliance can comprise are stainless steel or nickel-titanium wires having wire dimensions of:

| 0.0160″ square | 0.406 mm square |
|---|---|
| 0.0160″ × 0.0220″ | 0.406 mm × 0.559 mm |
| 0.0170″ square | 0.432 mm square |
| 0.0170″ × 0.0220″ | 0.432 mm × 0.559 mm |
| 0.0170″ × 0.0250″ | 0.432 mm × 0.635 mm |
| 0.0180″ square | 0.457 mm square |
| 0.0180″ × 0.0220″ | 0.457 mm × 0.559 mm |
| 0.0180″ × 0.0250″ | 0.457 mm × 0.635 mm |
| 0.0190″ square | 0.483 mm square |
| 0.0190″ × 0.0250″ | 0.483 mm × 0.635 mm |
| 0.0200″ square | 0.508 mm square |
| 0.0210″ × 0.0250″ | 0.533 mm × 0.635 mm |

Nickel-titanium closed or open-coil springs can be used. Some examples can include an elastomeric power chain, which can be capable of providing 100-800 grams of force, or intra-arch elastics. In some embodiments, the functional appliance or the orthodontic appliance comprises an elastic material. A functional appliance or an orthodontic appliance can exert a force on one or more teeth of the patient. The functional appliance or orthodontic appliance can cause one or more teeth to move or maintain its position. A functional appliance can cause bone remodeling of an oral or maxillofacial bone, or one or more tooth, such as a mandibular bone, maxillary bone, or temporal bone.

Installing, adjusting, or removing of an appliance, such as a functional appliance or orthodontic appliance, can occur before or after administering an effective dosage of light. In some embodiments, the effective amount of light aids in regulating or accelerating the movement of teeth during orthodontic treatment with an orthodontic appliance, or regulating or accelerating bone remodeling during oral or maxillofacial bone remodeling with a functional appliance. The effective amount of light can be useful for reducing the amount of time an orthodontic appliance is worn during an orthodontic treatment, or that a functional appliance is worn during treatment for oral or maxillofacial bone remodeling. For example, according to the methods of the present invention, the application of light can reduce treatment time (e.g., wearing a functional appliance or orthodontic appliance) by up to about 10%, about 20%, about 30%, about 40%, about 50%, about 60%, about 70%, about 75%, about 80%, about 85%, or about 90% of the treatment time. For example, administering light having a wavelength in the range of about 585 nm to about 665 nm (e.g., about 625 nm) can reduce the amount of time that a patient wears appliances (e.g., functional appliances or orthodontic appliance) by about 5% to about 90%, for example, by about 5%, about 10%, about 20%, about 30%, about 40%, about 50%, about 60%, about 70%, about 75%, about 80%, about 85%, or about 90%. Administering light having a wavelength in the range of about 815 nm to about 895 nm (such as, for example, about 855 nm) can reduce the amount of time that a patient wears appliances (e.g., functional appliances or orthodontic appliances) by about 5% to about 90%, for example, by about 5%, about 10%, about 20%, about 30%, about 40%, about 50%, about 60%, about 70%, about 75%, about 80%, about 85%, or about 90%.

In some embodiments, administering an effective amount of light with desired light characteristics results in the overall acceleration of treatment. For example, a treatment can include the installation of a functional appliance, the removal of the functional appliance, and the installation of an orthodontic appliance. By combining the use of a functional appliance and an orthodontic appliance, the overall treatment time can be reduced. Furthermore, increased control on the bone remodeling and tooth movement can be delivered. This can be particularly advantageous during a patient's adolescent growth phase.

Administering light having a wavelength in the range of about 585 nm to about 665 nm (such as, for example, about 625 nm) can result in a rate of bone remodeling that is about 5% to about 90% faster than the rate of bone remodeling without the administration of light. For example, the rate of bone remodeling can be about 5%, about 10%, about 20%, about 30%, about 40%, about 50%, about 60%, about 70%, about 75%, about 80%, about 85%, or about 90% faster than the rate of bone remodeling without the administration of light. Similarly, administering light having a wavelength in the range of about 585 nm to about 665 nm (such as, for example, about 625 nm) can result in a rate of tooth movement that is about 5% to about 90% faster than the rate of tooth movement without the administration of light. For example, the rate of tooth movement can be about 5%, about 10%, about 20%, about 30%, about 40%, about 50%, about 60%, about 70%, about 75%, about 80%, about 85%, or about 90% faster than the rate of tooth movement without the administration of light.

Administering light having a wavelength in the range of about 815 nm to about 895 nm (such as, for example, about 855 nm) can result in a rate of bone remodeling that is about 5% to about 60% faster than the rate of bone remodeling resulting from the administration of light having a wavelength in the range of 585 nm to about 665 nm (such as, for example, about 625 nm). In one example, the rate of bone remodeling can be about 5%, about 10%, about 20%, about 30%, about 40%, about 50%, about 55%, or about 60% faster than the rate of bone remodeling resulting from the administration of light having a wavelength in the range of 585 nm to about 665 nm (such as, for example, about 625 nm). Similarly, administering light having a wavelength in the range of about 815 nm to about 895 nm (such as, for example, about 855 nm) can result in a rate of tooth movement that is about 5% to about 60% faster than the rate of tooth movement resulting from the administration of light having a wavelength in the range of 585 nm to about 665 nm (e.g., about 625 nm). In one example, the rate of tooth movement can be about 5%, about 10%, about 20%, about 30%, about 40%, about 50%, about 55%, or about 60% faster than the rate of tooth movement resulting from the administration of light having a wavelength in the range of 585 nm to about 665 nm (e.g., about 625 nm).

Administering light having a wavelength in the range of about 815 nm to about 895 nm (such as, for example, about 855 nm) can result in a rate of bone remodeling that is about 5% to about 95% faster than the rate of bone remodeling without the administration of light. For example, the rate of bone remodeling can be about 5%, about 10%, about 20%, about 30%, about 40%, about 50%, about 60%, about 70%, about 75%, about 80%, about 85%, about 90%, or about 95% faster than the rate of bone remodeling without the administration of light. Similarly, administering light having a wavelength in the range of about 815 nm to about 895 nm (such as, for example, about 855 nm) can result in a rate of tooth movement that is about 5% to about 95% faster than the rate of tooth movement without the administration of light. For example, the rate of tooth movement can be about 5%, about 10%, about 20%, about 30%, about 40%, about 50%, about 60%, about 70%, about 75%, about 80%, about 85%, about 90%, or about 95% faster than the rate of tooth movement without the administration of light.

Orthodontic treatments, particularly those that comprise the use of an orthodontic appliance, can exert forces, such as heavy forces, on one or more teeth. This can result in a rate of tooth movement that is about 5% to about 80% faster than the rate of tooth movement without the exertion of heavy forces. For example, the exertion of heavy forces in one or more teeth can increase the rate of tooth movement by about 5%, about 10%, about 20%, about 30%, about 40%, about 50%, about 60%, about 70%, about 75%, or about 80%. Heavy forces can result in tooth-root resorption, bone resorption, inflammatory resorption of dentin, cementum resorption, or tissue inflammation.

In some embodiments, the administration of an effective amount of light can aid in reducing, preventing or minimizing tooth-root resorption when a heavy force is allowed to be exerted on one or more teeth. The effective amount of light can be useful for reducing the amount of tooth-root resorption as compared to when a heavy force is allowed to be exerted on one or more teeth without administering the effective amount of light. For example, according to the methods of the present invention, the administration of light can reduce tooth-root resorption by up to about 1%, about 2%, about 3%, about 5%, about 7%, about 10%, about 15%, about 20%, about 25%, about 30%, about 40%, about 50%, about 60%, about 70%, about 80%, or about 90%. Reducing tooth-root resorption, particularly while applying heavy forces, can allow for a reduction of the amount of time for orthodontic treatment, or the amount of time that a patient wears an orthodontic appliance. Administering an effective amount of light can reduce the amount of time that a patient wears orthodontic appliances by about 5% to about 90%, for example, by about 5%, about 10%, about 20%, about 30%, about 40%, about 50%, about 60%, about 70%, about 75%, about 80%, about 85%, or about 90%.

In some embodiments, administration of an effective amount of light can aid in reducing, preventing or minimizing bone resorption or inflammatory dentin or cementum resorption of the tooth root or periodontium. The effective amount of light can be useful for reducing bone resorption or inflammatory dentin or cementum resorption of the tooth root and periodontium, as compared to when a heavy force is allowed to be exerted on one or more teeth without administering the effective amount of light. For example, according to the methods of the present invention, the administration of light can reduce bone resorption or inflammatory dentin or cementum resorption of the tooth root or periodontium by up to about 1%, about 2%, about 3%, about 5%, about 7%, about 10%, about 15%, about 20%, about 25%, about 30%, about 40%, about 50%, about 60%, about 70%, about 80%, or about 90%. Reducing bone resorption or inflammatory resorption of dentin or cementum resorption of the tooth root or periodontium while exerting heavy forces can reduce the amount of time for orthodontic treatment, or amount of time that a patient wears an orthodontic appliance. Administering an effective amount of light can reduce the amount of time that a patient wears orthodontic appliances by about 5% to about 90%, for example, by about 5%, about 10%, about 20%, about 30%, about 40%, about 50%, about 60%, about 70%, about 75%, about 80%, about 85%, or about 90%.

In some embodiments, administration of the effective amount of light can aid in reducing, preventing or minimizing inflammation of tissue surrounding one or more teeth upon which heavy forces are or were exerted. The effective amount of light can be useful for reducing the amount of inflammation of tissue surrounding one or more teeth upon which heavy forces are or were exerted, as compared to when a heavy force is allowed to be exerted on one or more tooth without administering the effective amount of light. In one embodiment, according to the methods of the present invention, the administration of light can reduce inflammation of tissue surrounding one or more teeth upon which heavy forces are or were exerted by up to about 1%, about 2%, about 3%, about 5%, about 7%, about 10%, about 15%, about 20%, about 25%, about 30%, about 40%, about 50%, about 60%, about 70%, about 80%, or about 90%. Reducing inflammation of tissue surrounding one or more teeth upon which heavy forces are or were exerted while applying heavy forces can reduce the amount of time for orthodontic treatment, or amount of time that a patient wears an orthodontic appliance. Administering an effective amount of light can reduce the amount of time that a patient wears an orthodontic appliance by about 5% to about 90%, for example, by about 5%, about 10%, about 20%, about 30%, about 40%, about 50%, about 60%, about 70%, about 75%, about 80%, about 85%, or about 90%.

The light can be administered in accordance with a treatment regimen. For example, a functional appliance (or other orthodontic appliance) can be installed prior to, concurrently with, and/or subsequent to extra-orally or intra-orally administering the light. A functional appliance can also be removed prior to, concurrently with, and/or subsequent to extra-orally or intra-orally administering the light. Moreover, a functional appliance can be adjusted prior to, concurrently with, and/or subsequent to extra-orally or intra-orally administering the light.

The functional appliance can exert a force on oral or maxillofacial bone, soft tissue, or muscle, or one or more teeth, such as a mandibular bone, maxillary bone, or temporal bone of the patient. The functional appliance can exert the force subsequent to, concurrently with, or prior to the administration of light. The functional appliance can exert the force subsequent to, concurrently with, or prior to initiation of the administration of light. The functional appliance can exert the force subsequent to, concurrently with, or prior to the initiation of a light treatment regimen. The functional appliance can exert the force subsequent to, concurrently with, or prior to the initiation of a light treatment session. In some embodiments, the functional appliance exerts the force one or more seconds, one or more minutes, one or more hours, one or more days or one or more weeks subsequent to administering the light. The light can be administered for any length of time. In some embodiments, the functional appliance exerts the force one or more seconds, one or more minutes, one or more hours, one or more days or one or more weeks subsequent to initiating light administration. In some embodiments, the functional appliance exerts the force one or more seconds, one or more minutes, one or more hours, one or more days or one or more weeks subsequent to ending light administration.

Light can be administered for any period of time before, during, or after the functional appliance exerts the force. For example, light can be administered for about 1 minute, about 2 minutes, about 3 minutes, about 5 minutes, about 10 minutes, about 15 minutes, about 20 minutes, about 30 minutes, about 45 minutes, about 1 hour, about 90 minutes, about 2 hours, about 3 hours, about 4 hours, or about 6 hours prior to, during, or after the functional appliance exerts the force. In some embodiments, light is administered at any amount of time prior to, during, or after the initiation of the exertion of a force. For example, light can be administered about 1 minute, about 2 minutes, about 3 minutes, about 5 minutes, about 10 minutes, about 15 minutes, about 20 minutes, about 30 minutes, about 45 minutes, about 1 hour, about 90 minutes, about 2 hours, about 3 hours, about 4 hours, about 6 hours, about 12 hours, about 1 day, about 36 hours, about 2 days, about 3 days, about 4 days, about 1 week, about 2 weeks, or about 1 month prior to, during, or after the initiation of the exertion of a force.

Administering light prior to initiating or exerting a force, as described herein, can be part of a pretreatment regimen. In some embodiments, however, no such pretreatment occurs and the functional appliance exerts a force prior to any light being administered. The functional appliance can exert a force, for example, at one or more seconds, one or more minutes, one or more hours, one or more days or one or more weeks prior to administering the light. Thus, a follow-up treatment of light can be provided after the functional appliance exerts the force. In other embodiments, the functional appliance exerts the force during the administration of light as similarly described herein, or at one or more stages of the administration of light.

In some embodiments, the functional appliance exerts the force at the same region as the region that is administered with light. For example, the functional appliance can exert the force on a temporomandibular joint, and light can be administered to the temporomandibular joint. In other embodiments, the functional appliance exerts the force on a different region from the region that is administered with light. For example, the functional appliance can exert the force on one or more teeth, and light can be administered to one or both temporomandibular joint, condyle, or glenoid fossa. In some embodiments, allowing the functional appliance to exert a force on a region other than the region administered with light can result in allowing a force to be exerted to a region that is administered with light. For example, a functional appliance can exert a force on one or more teeth, causing a mandibular bone to be shifted correspondingly forward, which can result on a force being exerted at a mandibular joint, where a light can be administered.

In one embodiment, the effective dosage of light has a density that ranges from about 24 J/cm² to about 200 J/cm², and has a wavelength that ranges from about 585 nm to about 665 nm, from about 666 nm to about 814 nm, or from about 815 nm to about 895 nm. Administration of light having a wavelength in the range of about 585 nm to about 665 nm can be useful for promoting bone remodeling, bodily movement of teeth or minimize tipping of teeth, or any combination thereof. Administration of light having a wavelength in the range of 815 nm to about 895 nm can be useful for increasing the velocity of teeth through the patient's bone or the velocity of bone remodeling. Administration of light in the range of about 585 nm to about 665 nm, or about 815 nm to about 895 nm can be useful for promoting bone remodeling at one or more region of the mandibular or maxillary bone. In some other examples, an effective dosage of light can have any of the light characteristics as described herein.

Teeth in a region of the patient's maxillary or mandibular alveolar bone to which light is not administered can be used as an anchor to facilitate movement of teeth in the selected region. Teeth in a region of the patient's maxillary or mandibular alveolar bone, the mandibular bone, the maxillary bone, or any other oral or facial feature can be used as an anchor to facilitate bone remodeling in the mandibular bone or maxillary bone. In some embodiments the light is administered to the patient's face. In other embodiments, the light is administered directly to a specific region of the patient's maxillary or mandibular alveolar bone, one or both temporomandibular joint, one or both condyle, one or both glenoid fossa, or to any other region of a patient's mandibular bone, maxillary bone, or temporal bone. In some embodiments where light is administered directly to a specific region, the light reaches the specific region without first reflecting from another region.

In some embodiments, methods are provided for regulating bone remodeling, comprising extra-orally or intra-orally administering to a patient in need thereof an effective dosage of light having a first wavelength to a selected first region of the patient's maxillary bone, mandibular bone, or temporal bone, and extra-orally or intra-orally administering an effective dosage of light having a second wavelength to a selected second region of the patient's maxillary bone, mandibular bone, or temporal bone. In one embodiment, the regulating occurs prior to, subsequent to or concurrently with orthodontic treatment of a patient. In one embodiment, the effective dosage of light having a first wavelength is a repetitive dosage. In another embodiment, the effective dosage of light having a second wavelength is a repetitive dosage. Regions other than the maxillary, mandibular, or temporal bone can receive the first or second wavelength of light. In one embodiment, the effective dosage of light is in the range of 24 J/cm² to 200 J/cm². The first wavelength can be in the range of about 585 nm to about 665 nm, and the second wavelength can be in the range of about 666 nm to about 814 nm or about 815 nm to about 895 nm. In other examples, an effective dosage of light can have any light characteristics as described herein. In one embodiment the light is administered to the patient's face.

In some embodiments, methods for oral or maxillofacial bone remodeling can also result in regulating tooth movement, comprising extra-orally administering to a patient in need thereof an effective dosage of light having a first wavelength to a selected first region of the patient's maxillary bone, mandibular bone, or temporal bone and extra-orally administering an effective dosage of light having a second wavelength to a selected second region of the patient's maxillary bone, mandibular bone, or temporal bone. In one embodiment, regulating of oral or maxillofacial bone remodeling occurs prior to, subsequent to or concurrently with orthodontic treatment of a patient. In one embodiment, the effective dosage of light having a first wavelength is a repetitive dosage. In another embodiment, the effective dosage of light having a second wavelength is a repetitive dosage. Regions other than alveolar bone can receive the first or second wavelength of light. In one embodiment, the effective dosage of light is in the range of 24 J/cm² to 200 J/cm². The first wavelength can be in the range of about 585 nm to about 665 nm, and the second wavelength can be in the range of about 666 nm to about 814 nm or about 815 nm to about 895 nm. In other examples, an effective dosage of light can have any light characteristics as described herein. In one embodiment, the light is administered through the patient's face.

In some embodiments, the present methods comprise administering light until treatment for oral or maxillofacial bone remodeling is complete. Treatment for oral or maxillofacial bone remodeling can be deemed complete after appointments with an orthopedic or other specialist are completed, after the movement of a mandibular bone or maxillary bone has been stabilized to substantially remain in the same position without the aid of a functional appliance, or during a passive stage of treatment for oral or maxillofacial bone remodeling as described in greater detail herein. Light can be administered to the region before, during, after, or any combination thereof, a functional appliance is installed, adjusted, or removed. The functional appliance can be installed, adjusted, or removed before, during, after, or any combination thereof, the application of light. In some embodiments, a force can be exerted when the functional appliance is installed or adjusted, or for a period of time following such installation or adjustment.

In some embodiments, the methods for oral or maxillofacial bone remodeling are useful for orthodontic treatment. The methods can comprise installing an orthodontic appliance, removing an orthodontic appliance or adjusting an orthodontic appliance. In other embodiments, the methods comprise administering light until orthodontic treatment is complete. Orthodontic treatment can be deemed complete after appointments with an orthodontic specialist are completed, after the movement of one or more teeth has been stabilized to substantially remain in the same position without the aid of an orthodontic appliance, or during a passive stage of orthodontic treatment as described in greater detail herein. Light can be administered to the region before, during, after, or any combination thereof, an orthodontic appliance is installed, adjusted, or removed. The orthodontic appliance can be installed, adjusted, or removed before, during, after, or any combination thereof, the application of light. In some embodiments, a force can be exerted when the orthodontic appliance is installed or adjusted, or for a period of time following such installation or adjustment.

As described herein, the speed of bone remodeling, e.g., the repositioning, or altering of one or more dimensions of a bone, can be regulated (e.g., accelerated or decelerated) by the administration of light. In one embodiment, the present methods are useful for effecting bone remodeling, which can occur concurrently with regulating bone remodeling, such as tooth movement. Bone remodeling can be enhanced by administering light according to the present methods. The light can be administered before, during or after treatment for oral or maxillofacial bone remodeling. The light can be emitted from a light-therapy apparatus, such as described herein. Bone remodeling can include bone growth or bone resorption. This can include osteoblast or osteoclast activation. Bone remodeling can require osteoclastic and osteoblastic activity. In one embodiment, the administration of light according to the present methods stimulates osteoclasts or osteoblasts and, accordingly, stimulates osteoclastic and osteoblastic activity. The administration of light can increase the rate of tooth movement that can accompany bone remodeling.

For example, the present methods can also comprise applying, adjusting or removing a tooth mask or other oral mask. A tooth mask can be applied or removed prior to, during, or after the administration of light. Light can be administered to a region before, during, after, or any combination thereof, an oral mask or tooth mask is applied, adjusted, or removed. In some embodiments, one or more of a patient's teeth, or other region of the patient's mouth or face, or other region of the patient's body, such as the patient's oral cavity, can be at least partially covered with a mask that can block at least some of the light. A mask can block one or more wavelengths of light. In some embodiments, a mask can completely block one or more wavelength of light, and in other embodiments, the mask can reduce the amount or intensity of light reaching the teeth or other region of the patient's mouth, face, or body. In some embodiments, the intensity of the light administered to the teeth, or other region of the patient's mouth, face, or body, can be zero, or can be less than the intensity of the light emitted from a light source.

In accordance with another aspect of the invention, the methods for tooth movement regulation can regulate the bone remodeling. For example, the methods for tooth movement regulation can increase the rate of bone remodeling. In some embodiments, bone remodeling can facilitate or otherwise aid in tooth movement regulation (e.g., can increase the velocity or quality of movement, or can stabilize tooth movement). For example, bone remodeling can occur prior to, during or following tooth movement. Bone remodeling can include bone growth, bone strengthening or bone resorption. For example, during bone remodeling, bone mineral density (BMD) can increase, bone volume (BV) can increase, bone mineral content (BMC) can increase, and the ratio of bone volume to total volume (BV/TV) or bone density can increase. Higher BV/TV can indicate denser bone, where less bone remodeling can occur, which is desirable after bone remodeling or tooth movement has occurred to enhance the stability, for example, of the maxillary bone or mandibular bone or teeth. Other examples of parameters that can be affected during bone remodeling can include trabecular bone surface, bone quality, osteoclastic activity (e.g., osteoclast and preosteoclast counts), bone resorption. Light therapy can enhance existing cellular processes. Bone remodeling can occur in any bone tissue or oral region. For example, bone remodeling can occur in a portion or all of a maxillary alveolar bone, in mandibular alveolar bone, around one or more teeth, a temporomandibular joint, a condyle, a glenoid fossa, or any other mandibular bone, maxillary bone, or temporal bone. In some embodiments, bone remodeling can occur around one or more teeth, which can include a periodontium. In some embodiments, the region around one or more teeth can be within about 1 mm, about 2 mm, or about 3 mm from the surface of the teeth.

In some embodiments, light therapy according to the present methods can also result in treating or preventing jaw osteonecrosis. Accordingly, the present methods are useful for treating or preventing jaw osteonecrosis. Accordingly, the invention further provides methods for treating or preventing jaw osteonecrosis, comprising extra-orally administering to a patient in need thereof an effective amount of light transdermally to a region of the patient's maxillary or mandibular alveolar bone. Treating or preventing jaw osteonecrosis can comprise reversing osteonecrosis through the use of light therapy according to the methods described herein. Jaw osteonecrosis can occur with respect to any bone tissue. For example, jaw osteonecrosis, can occur with respect to a portion or all of a maxillary alveolar bone, mandibular alveolar bone, or one or more teeth. In some embodiments, methods for treating or preventing jaw osteonecrosis further comprise administering to the patient an effective amount of vitamin D.

In some embodiments, light therapy according to the present methods can also result in reducing, minimizing, or preventing tooth-root resorption, bone resorption, inflammatory resorption of dentin or cementum resorption, or inflammation of tissue. Accordingly, the present methods are useful for reducing, minimizing, or preventing tooth-root resorption, bone resorption, inflammatory dentin or cementum resorption, or inflammation of tissue. Accordingly, the invention further provides methods for reducing, minimizing, or preventing tooth-root resorption, bone resorption, inflammatory dentin or cementum resorption, or inflammation of tissue, comprising allowing a force to be exerted on one or more teeth of a patient in need thereof; and administering an effective amount of light to the maxillary or mandibular alveolar bone of the patient, wherein the light is administered before, during, or after the force is exerted. Such methods can be used or useful in conjunction with forces applied to one or more tooth. In some embodiments, methods for reducing, minimizing, or preventing tooth-root resorption, bone resorption, inflammatory resorption of dentin or cementum resorption, or inflammation of tissue further comprise administering to the patient an effective amount of vitamin D. In some embodiments, the region to which light is administered is any oral tissue, such as soft tissue or bone tissue. In some embodiments, the oral tissue is that on which oral surgery was performed. The present methods are useful for treating tissue after oral surgery. The oral surgery can be periodontal surgery or that relating to bone grafts. The oral tissue can be: a portion or all of tissue supporting one or more teeth, the gums, a maxillary alveolar bone, mandibular alveolar bone, or one or more teeth. Accordingly, the invention further provides methods for treating tissue after oral surgery, comprising extra-orally administering to a patient in need thereof an effective amount of light transdermally to a region of the patient's oral tissue on which oral surgery was performed. The present methods are also useful for increasing the rate of oral-tissue healing following oral surgery. Accordingly the invention further provides methods for increasing the rate of oral-tissue healing following oral surgery, comprising extra-orally administering to a patient in need thereof an effective amount of light transdermally to a region of the patient's oral tissue on which oral surgery will be performed. In some embodiments, the methods further comprise performing oral surgery on the oral tissue. The oral surgery can be performed prior to or subsequent to the administration of light therapy according to the present methods. In some embodiment, the region of light administration can be the alveolar bone. In some embodiments, the light administration occurs extra-orally, and light is transdermally administered to the region. In some embodiments, the light administration can occur intra-orally, and the light can be directly administered to the region. In some embodiments, the administration occurs for about 20 minutes. In some embodiments, the wavelength of administered light is about 625 nm. In some embodiments, the light can be administered following oral surgery, prior to oral surgery, or during oral surgery.

In other embodiments, the invention relates to methods for healing tissue surrounding or adjacent to one or more dental implants, for example, endosseous dental implants, or accelerating osseo-integration of endosseous dental implants, comprising extra-orally administering to a patient in need thereof an effective amount of light transdermally to a region of the patient's maxillary or mandibular alveolar bone. In other embodiments, the methods comprise intra-orally administering to a patient in need thereof an effective amount of light to a region of the patient's maxillary or mandibular alveolar bone. In one embodiment, these methods can be performed according to the teachings disclosed herein for the methods for regulating tooth movement.

In some embodiments, the present methods can further comprise applying a substance to a region, or in the proximity of a region, before, during, or after the administration of light. In some embodiments the methods can exclude the application of a substance to a region, or in the proximity of a region, before, during, or after the administration of light, or before, during, or after the exertion of forces. In some embodiments, a substance can already occur at a region naturally. In some embodiments, the methods can optionally comprise applying a substance to at least a portion of the face overlying a region before, during, or after the administration of light. In some embodiments the methods for regulating bone remodeling, such as methods for regulating tooth movement, can exclude the application of a substance to at least a portion of the face overlying a region before, during, or after the administration of light. Optionally, light can be administered before, during, or after the administration of a substance. In some embodiments, light is administered only without the administration of a substance. The substance can enhance or inhibit the effects of the light administration. In one embodiment, the substance is a visible-light- or infrared-light-absorbing substance, such as a dye. One or more light characteristics, such as wavelength of light, can be selected in response to the presence or application of the substance.

Vitamin D

The present methods can further comprise administering vitamin D. Vitamin D is essential for normal bone metabolism—it promotes calcium absorption and bone resorption and maintains the necessary calcium and phosphate levels for bone formation. Patients deficient in vitamin D have an increased risk of bone loss and bone fracture, among many other risks. Insufficient vitamin D levels can also interfere with osteoclastic activity, which is essential to tooth movement, resulting in slower tooth movement. Thus, administering vitamin D can be an important part of orthodontic treatment.

The vitamin D can be vitamin D1, D2, D3, D4, D5, 1,25-dihydroxycholecalciferol, or mixtures thereof. In some embodiments, the vitamin D supplements other vitamin D sources for the patient.

The vitamin D can be administered orally, via transdermal gel, by a patch, by a cream, by injection, by electrophoresis, or by insolation. Where the present methods further comprise administering vitamin D, in some embodiments, the vitamin D is not administered by insolation. In some embodiments, the vitamin D is administered via a vitamin D conveyance. For example, the vitamin D can be present in a composition suitable for oral administration, for example, a pill, capsule, tablet, chewable, gel, or liquid. In other embodiments, the vitamin D is administered transdermally. In one example, the vitamin D can be administered transdermally via a transdermal gel, cream, ointment, liquid, or paste that can be applied to the skin, gums, or any soft tissue. In another example, vitamin D can be administered transdermally via insolation, such as exposure to ultraviolet (UV) rays from the sun or artificially through tanning beds. The vitamin D can also be administered transdermally via a patch or microneedle on the skin, gums, or other soft tissue of the patient. In some embodiments, the vitamin D is be administered by injection using a syringe or needle at the skin, gums, or other soft tissue (such as, for example, oral tissue) of the patient. The injection can be intradermal, subcutaneous, intramuscular, intravenous, intraosseous, or intraperitoneal. In some embodiments, the vitamin D is administered electrophetically. The vitamin D can be applied, for example, to the surface of the skin, gums, or any other soft tissue, and a weak electrical current can drive the compound through the tissue.

Any combination of the various vitamin D administration techniques described herein can be employed. For example, a patient can be orally administered with vitamin D also receive an injection of vitamin D as part of the administration process. In some embodiments, the administered vitamin D increases or maintains the vitamin D blood serum levels. In other embodiments, the administered vitamin D increases or maintains local vitamin D levels where the vitamin D is administered.

In some embodiments, the vitamin D is administered to a region, or in the proximity of a region. The region can be, for example, an oral region. The region can be, for example, on or in the proximity of oral or maxillofacial bone, muscle, or soft tissue. The region can be on or in the proximity of one or more tooth, the mandibular bone, the maxillary bone, or the temporal bone. In some embodiments, the vitamin D is orally administered, for example, via an oral composition that comprises vitamin D. In other embodiments, the vitamin D is administered locally to a region. The region can be on the skin of the patient overlying the patient's face, jawbone, lips, cheek, or chin. The region can be on the right side, the left side, a central region, or any combination thereof, of the patient's body such as, for example, the patient's face. The region can be within the patient's oral cavity. For example, the region can be the gums of the patient, or any other oral soft tissue. The region need not be an oral region; rather, the region can be, for example, on the neck, arm, leg, or torso of the patient. In some embodiments, the vitamin D can be administered systemically to the patient. For example, the vitamin D can be administered via insolation through a tanning bed that surrounds the patient's body. The region can include any area previously described.

In some embodiments, the vitamin D is administered to a region that is the same as or in the proximity of a region that is administered with light. In some embodiments, the vitamin D is administered to the same region that is administered with light. In some other embodiments, the vitamin D is administered to a region having the same, greater, or smaller size than the region administered with light. The vitamin D can be administered to a region adjacent to a region administered with light. In some embodiments, vitamin D is administered to a region within about 1 mm, about 2 mm, about 3 mm, about 5 mm, about 7 mm, about 10 mm, about 15 mm, about 20 mm, about 25 mm, about 30 mm, about 40 mm, about 50 mm, about 60 mm, about 70 mm, about 10 cm, about 15 cm, about 20 cm, about 30 cm, about 50 cm from a region that is administered with light. In other embodiments, the vitamin D is administered to a region that different from the region that is administered with light. In some embodiments vitamin D is not administered to a region that is administered with light. In some embodiments, vitamin D is administered to a region other than the region that is administered with light. In some embodiments, vitamin D is administered systemically, which can encompass the region administered with light. In some instances, the vitamin D is administered systemically, raising overall vitamin D levels, which can include vitamin D levels in the region administered with light.

In some embodiments, the vitamin D is administered to a region that is proximate to a region upon which a force is exerted. The force can be, for example, a heavy force, a force exerted by an orthodontic appliance, or a force exerted by a functional appliance. In some embodiments, the vitamin D is administered to the same region upon which a force is exerted. In some embodiments, the region where the vitamin D is administered and the region upon which the force is exerted are the same size. In other embodiments, however, the size of the region where the vitamin D is administered is different from the size of the region upon which the force is exerted. The region where the vitamin D is administered can be, for example, smaller or larger than the region upon which the force is exerted. In some embodiments, the vitamin D is administered to a region adjacent to a region upon which a force is exerted. The vitamin D can be administered to a region, for example, within about 1 mm, about 2 mm, about 3 mm, about 5 mm, about 7 mm, about 10 mm, about 15 mm, about 20 mm, about 25 mm, about 30 mm, about 40 mm, about 50 mm, about 60 mm, about 70 mm, about 10 cm, about 15 cm, about 20 cm, about 30 cm, about 50 cm of a region upon which a force is exerted.

In some embodiments, the vitamin D is administered to a region that is different from the region upon which a force is exerted. In other words, the vitamin D is not administered to a region upon which a force is exerted. In some embodiments, vitamin D is administered systemically and can encompass the region upon which a force is exerted. For example, in some instances, the vitamin D is administered systemically and raises overall vitamin D levels, including the vitamin D levels in the region upon which a force is exerted.

The present methods can include administering an effective amount of vitamin D to a patient in need thereof, and administering an effective amount of light to oral or maxillofacial bone, muscle, or soft tissue, or one or more teeth of the patient. The present methods can include administering an effective amount of light to the maxillary or mandibular alveolar bone of the patient. In some embodiments, the effective amount of vitamin D can be administered to an oral region of the patient. Alternatively, the effective amount of vitamin D can be administered systemically to the patient. The light can be intra-orally or extra-orally administered. In some embodiments, the method can further comprise testing the patient to determine his or her vitamin D level. For example, the patient can undergo blood testing to determine the patient's vitamin D level. If necessary, a patient can receive a vitamin D supplement or treatment. Light can be administered to the alveolar mucosa and/or teeth in conjunction with orthodontic treatment and normal or higher vitamin D levels, which can accelerate orthodontic tooth movement.

The present methods can comprise administering an effective amount of vitamin D to a patient and providing a light therapy apparatus. The light therapy apparatus can be a light therapy apparatus as described in further detail below. The methods can optionally comprise determining whether the patient is vitamin D deficient. The methods can optionally comprise measuring the patient's vitamin D blood serum level. In some embodiments, if the patient's vitamin D blood serum level is below a predetermined threshold, the patient can administer or be administered with a dosage of vitamin D. In some embodiments, the dosage of vitamin D is determined based on the patient's blood serum level and administered to the patient. The dosage of vitamin D to be administered to the patient can be determined, for example, based on the patient's blood serum level, so that the patient is administered with an effective amount of vitamin D. For example, if the patient is very deficient in vitamin D (i.e., has very low vitamin D blood serum levels), the patient can receive a greater dosage of vitamin D than if the patient is only slightly deficient in vitamin D (i.e., has higher vitamin D blood serum levels). In other embodiments, regardless of the vitamin D blood serum level, if the patient is vitamin D deficient, the patient receives the same vitamin D dosage. In yet other embodiments, a dosage of vitamin D is administered to the patient even if the patient is not vitamin D deficient. In embodiments where the patient is vitamin D deficient, the length of vitamin D treatment can vary depending on the degree of vitamin D deficiency.

The vitamin D can be administered in one or more dosages. In some embodiments, as described herein, a dosage of vitamin D is an effective amount of vitamin D. In other embodiments, a single dosage of vitamin D can be greater than or less than an effective amount of vitamin D. A dosage of vitamin D can be provided for a period of time. For example, the vitamin D can be administered daily. In some embodiments, the vitamin D is administered every hour, several times a day, once a day, once every several days, once a week, once every few weeks, once a month, once every few months, once a quarter, or with any other frequency. Vitamin D can be administered on a regular basis (e.g., every 6 hours, every day, every 10 days), or can be provided at irregular intervals (e.g., twice one day, skip a day, once the next day). In some embodiments, vitamin D is administered on an as-needed basis.

In some embodiments, the dosage is greater than about, is less than about, or is about 100 IU, about 200 IU, about 400 IU, about 500 IU, about 600 IU, about 800 IU, about 1,000 IU, about 1,200 IU, about 1,500 IU, about 1,600 IU, about 2,000 IU, about 2,500 IU, about 3,000 IU, about 4,000 IU, about 5,000 IU, about 6,000 IU, about 7,000 IU, about 8,000 IU, about 9,000 IU, about 10,000 IU, about 12,000 IU, about 15,000 IU, about 17,000 IU, about 20,000 IU, about 25,000 IU, about 30,000 IU, about 40,000 IU, about 50,000 IU, about 70,000 IU, about 100,000 IU, about 150,000 IU, about 200,000 IU, about 300,000 IU, about 400,000 IU, about 500,000 IU, about 600,000 IU, or about 800,000 IU. In some embodiments, the dosage amount varies each time the vitamin D is administered to the patient. In other embodiments, the dosage amount is a daily amount of vitamin D administered to the patient. In other embodiments, the dosage amount is the total vitamin D amount administered for a treatment regimen. For example, a daily oral dosage of vitamin D can range from 400 IU to 6,000 IU per day. In another example, a daily oral dosage of vitamin D can range from 2,000 IU to 6,000 IU per day. A daily oral supplement of 2,000 IU to 6,000 IU of vitamin D in adults has been shown to increase blood levels of vitamin D to 40 ng/mL within 3 months. In some regimens, higher initial dosages of vitamin D have shown increases in vitamin D blood levels. The dosage of vitamin D can be a single dose of 600,000 IU of oral vitamin D. Based on one clinical trial, a single dose of 600,000 IU of oral vitamin D was comparable to a dose of 20,000 IU per day of oral vitamin D for 30 days. In another embodiment, the dosage is 20,000 IU per day of oral vitamin D for 30 days.

The dosage of vitamin D can be sufficient to raise the vitamin D blood level between about 40 to about 60 ng/mL of venous blood. The dosage of vitamin D can be sufficient to raise vitamin D blood level to at least about, no more than about, or to about 20 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, or about 80 ng/mL. In some embodiments, the dosage of vitamin D is sufficient to raise the vitamin D blood level by any amount. For example, the dosage of vitamin D can be sufficient to raise the vitamin D blood level by about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, or about 60 ng/mL. The vitamin D blood level can be raised to a desired level or by a desired amount within a period of time. For example, the period of time can be within one or more days, one or more weeks, one or more months, or one or more years. For example, a dosage of vitamin D administered daily can raise vitamin D blood serum levels to a desired level within 30 days, or within 3 months.

Vitamin D can be administered to the patient prior to, concurrently with, or subsequent to administering light therapy to the patient. Vitamin D can be administered to the patient prior to initiation of the light therapy administration, or prior to the completion of the light therapy administration. In some embodiments, a dosage of vitamin D is administered at a period of time (e.g., seconds, minutes, hours, days, weeks, months) prior to initiation of the light therapy administration or prior to completion of the light therapy administration. In some embodiments, a dosage of vitamin D is administered at a period of time (e.g., seconds, minutes, hours, days, weeks, months) subsequent to initiation of the light therapy administration or subsequent to completion of the light therapy administration. In some embodiments, a vitamin D treatment regimen (which can span one or more doses of vitamin D) is initiated or completed prior to initiation of light therapy administration or prior to completion of light therapy administration. In other embodiments, the vitamin D treatment regimen is initiated or completed subsequent to the initiation of light therapy administration or subsequent to completion of light therapy administration. The vitamin D treatment regimen can be in progress during light therapy administration.

Vitamin D can be administered to the patient prior to, currently with, or subsequent to engaging a light therapy apparatus with the patient. Vitamin D can also be administered to the patient prior to removing a light therapy apparatus from the patient. In some embodiments, a dosage of vitamin D can be administered at a period of time (e.g., seconds, minutes, hours, days, weeks, months) prior to engaging a light therapy apparatus with the patient or prior to removing a light therapy apparatus from the patient. In some embodiments, a dosage of vitamin D is administered at a period of time (e.g., seconds, minutes, hours, days, weeks, months) subsequent to engaging a light therapy apparatus with the patient or subsequent to removing a light therapy apparatus from the patient. In some embodiments, a vitamin D treatment regimen (which can span one or more doses of vitamin D) is initiated or completed prior to engaging a light therapy apparatus with the patient or prior to removing a light therapy apparatus from the patient. In other embodiments, the vitamin D treatment regimen is initiated or completed subsequent to engaging a light therapy apparatus with the patient or subsequent to removing a light therapy apparatus from the patient. The vitamin D treatment regimen can be in progress during light therapy administration.

Vitamin D can be administered to the patient prior to, currently with, or subsequent to exerting a force on one or more teeth of the patient. The force can be, for example, a heavy force, a force exerted by an orthodontic appliance, or a force exerted by a functional appliance. In some embodiments, the force can be less than a heavy force. In some embodiments, the vitamin D is administered to the patient prior to initiation of exerting a force on one or more teeth of the patient, or prior to the completion of exerting a force on one or more teeth of the patient. In some embodiments, a dosage of vitamin D is administered at a period of time (e.g., seconds, minutes, hours, days, weeks, months) prior to initiation of exerting a force on one or more teeth of the patient or prior to completion of exerting a force on one or more teeth of the patient. In other embodiments, a dosage of vitamin D is administered at a period of time (e.g., seconds, minutes, hours, days, weeks, months) subsequent to initiation of exerting a force on one or more teeth of the patient or subsequent to completion of exerting a force on one or more teeth of the patient. In some embodiments, a vitamin D treatment regimen (which can span one or more doses of vitamin D) is initiated or completed prior to initiation of exerting a force on one or more teeth of the patient or prior to completion of exerting a force on one or more teeth of the patient. In other embodiments, the vitamin D treatment regimen is initiated or completed subsequent to the initiation of exerting a force on one or more teeth of the patient or subsequent to completion of exerting a force on one or more teeth of the patient. The vitamin D treatment regimen can be in progress while exerting a force on one or more teeth of the patient.

Vitamin D can be administered to the patient prior to, concurrently with, or subsequent to installing one or more orthodontic appliances on the patient's teeth. In some embodiments, the vitamin D is administered to the patient prior to removing one or more orthodontic appliances from the patient's teeth. In some embodiments, a dosage of vitamin D is administered at a period of time (e.g., seconds, minutes, hours, days, weeks, months) prior to installing one or more orthodontic appliances on the patient's teeth or prior to removing one or more orthodontic appliances from the patient's teeth. In other embodiments, a dosage of vitamin D is administered at a period of time (e.g., seconds, minutes, hours, days, weeks, months) subsequent to installing one or more orthodontic appliances on the patient's teeth or subsequent to removing one or more orthodontic appliances from the patient's teeth. In some embodiments, a vitamin D treatment regimen (which can span one or more doses of vitamin D) is initiated or completed prior to installing one or more orthodontic appliances on the patient's teeth or prior to removing one or more orthodontic appliances from the patient's teeth. In other embodiments, the vitamin D treatment regimen is initiated or completed subsequent to the installing one or more orthodontic appliances on the patient's teeth or subsequent to removing one or more orthodontic appliances from the patient's teeth. The vitamin D treatment regimen can be in progress while an orthodontic appliance is installed on the patient's teeth.

The administration of vitamin D can increase the amount of tooth movement compared to treatment methods where vitamin D is not administered. The administration of vitamin D can also increase the rate of tooth movement compared to treatment methods where vitamin D is not administered. In some embodiments, the administration of vitamin D increases the velocity of tooth movement by about 5%, about 10%, about 15%, about 20%, about 25%, about 30%, about 40%, about 50%, about 60%, about 70%, about 80%, about 90%, or by any percentage falling within about 1% to about 90%, relative to treatment methods for regulating tooth movement that do not comprise administering vitamin D. In some embodiments, the administration of vitamin D increases the rate of bone remodeling compared to treatment methods where vitamin D is not administered. In some embodiments, the administration of vitamin D increases the velocity of bone remodeling by about 5%, about 10%, about 15%, about 20%, about 25%, about 30%, about 40%, about 50%, about 60%, about 70%, about 80%, about 90%, or by any percentage falling within about 1% to about 90%, relative to treatment methods for regulating bone remodeling that do not comprise administering vitamin D.

The administration of vitamin D can reduce the amount of time that the patient undergoes orthodontic treatment. The administration of vitamin D can also reduce the amount of time that a force is exerted on one or more teeth of the patient. In some embodiments, the administration of vitamin D reduces the amount of time that a patient undergoes orthodontic treatment or that a force is exerted on one or more teeth of the patient by about 5%, about 10%, about 15%, about 20%, about 25%, about 30%, about 40%, about 50%, about 60%, about 70%, about 80%, about 90%, or by any percentage falling within about 1% to about 90%, relative to treatment methods that do not comprise administering vitamin D.

The administration of vitamin D can increase the rate of bone remodeling compared to treatment methods where vitamin D is not administered. The administration of vitamin D can also increase the rate of one or both of bone deposition and resorption compared to treatment methods where vitamin D is not administered. In some embodiments, the administration of vitamin D increases the rate of one or both of bone deposition or resorption by about 5%, about 10%, about 15%, about 20%, about 25%, about 30%, about 40%, about 50%, about 60%, about 70%, about 80%, about 90%, or by any percentage falling within about 1% to about 90%, relative to treatment methods that do not comprise administering vitamin D.

Light Therapy Apparatuses and Systems

An aspect of the invention relates to light-therapy apparatuses. The light-therapy apparatuses are useful for administering an effective amount of light (for example, to the oral or maxillofacial bone, muscle, or soft tissue or to one or more teeth of a patient) and, accordingly, useful in the present methods for regulating bone remodeling or tooth movement. The light-therapy apparatuses are also useful for regulating movement of teeth: for reducing, minimizing or preventing tooth-root resorption; for reducing, minimizing or preventing bone resorption or inflammatory dentin or cementum resorption of a tooth root or periodontium; for reducing, preventing or minimizing inflammation of tissue surrounding one or more teeth upon which forces are or were exerted; for performing craniofacial surgery; for performing oral or maxillofacial surgery; for performing orthognathic surgery: for bone remodeling; or for treating or preventing jaw osteonecrosis, periodontitis, or malocclusion. Apparatuses and systems as described herein can also be applied to treat a variety of conditions including: conditions treated by orthodontics, conditions treated by orthopedics, application of forces on one or more teeth, stimulation and acceleration of healing after oral surgery or periodontal surgery, stimulation of the healing of wounds at the locations of bone grafts, healing and acceleration of ossco-integration of endosscous dental implants; or any other applications as described elsewhere herein. In one embodiment, the application to jaw osteonecrosis permits treatment of a condition for which existing treatments are highly invasive. Treating osteonecrosis using light therapy is significantly more cost-effective and comfortable for the patient than existing surgical treatment options. A light-therapy apparatus useful for methods for regulating bone remodeling, tooth movement or other methods described herein, can have other effects. For example, extra-oral administration of light to the condylar portion of the mandible can increase its growth or cause its expansion.

A light therapy system is provided and comprises a light-therapy apparatus. A light therapy system can also optionally comprise an oral appliance, such as an orthodontic appliance, or oral or tooth mask. In some embodiments, the orthodontic appliance can be a functional appliance. Any orthodontic appliance, including any functional appliance, as described herein, can be part of the light therapy system. An oral or tooth mask can block or partially filter one or more wavelength of light from a region covered by the mask. For example, a tooth mask can cover one or more teeth. The tooth mask can cover one or more mandibular or maxillary tooth. An oral mask can cover any region of the mouth. For example, an oral mask can cover one or more teeth, or one or more portion of the gums. An oral mask or tooth mask can be formed of a transparent, translucent, or opaque. An oral mask or tooth mask can block all wavelengths, reduce the intensity of all wavelengths, filter only some wavelengths, or reduce the intensity of only some wavelengths. In some embodiments, an oral mask or tooth mask can alter one or more light characteristics.

A light therapy system can also optionally include an external controller or a computer (or any other device described below) in communication with a controller.

Any embodiments of a light-therapy apparatus as described herein can be incorporated within the light therapy system. The light-therapy apparatus can optionally comprise one or more support features that can engage with a portion of a patient's face or head. In another embodiment the light-therapy apparatus engages with the mouth of the patient. The light-therapy apparatus can also comprise one or more light sources, wherein the one or more light sources can each comprise one or more light emitters. The light therapy system can also comprise a controller that controls the operation of the light-therapy apparatus. The controller can control the wavelength, intensity or duration of light emitted by the light-therapy apparatus or the position of its components. The controller can control any other light characteristics. The controller can be integral to or separate from the light-therapy apparatus. The light therapy system provides light and, accordingly, is useful in the present methods.

In some embodiments, a light therapy system comprises one or more other appliances. For example, a functional appliance can be installed within or external to an oral cavity of the patient. In another embodiment, an oral mask or tooth mask can be applied within the oral cavity of the patient. A light therapy system can include oral appliances or inserts that are within the oral cavity of the patient.

The light-therapy apparatus can be fixed or movable with respect to the functional appliance, oral or tooth mask, or any other appliance.

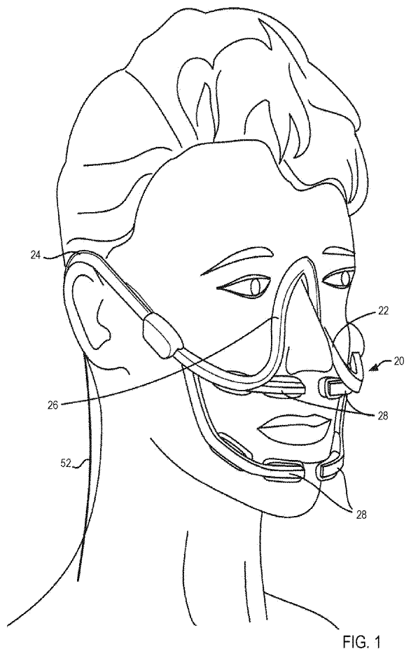

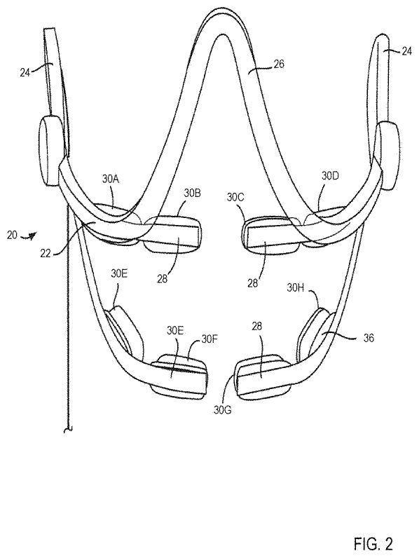

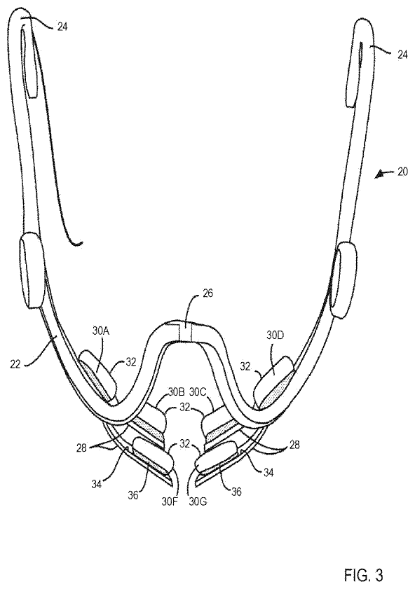

An embodiment of a light-therapy apparatus 20 is shown in FIGS. 1-4. FIG. 1 is an isometric view of an embodiment of a light-therapy apparatus useful for providing light to one or more specified regions of a patient's maxillary or mandibular bone. FIG. 2 is a front view of the embodiment shown in FIG. 1. FIG. 3 is a top view of the embodiment shown in FIG. 1. FIG. 4 is a right isometric view of the embodiment shown in FIG. 1. The light-therapy apparatus can be useful for providing light to any region described herein.

Light-therapy apparatus 20 has a frame 22 which is sized and/or shaped to engage with one or more features of a patient's face. Features of a patient's face can include, but are not limited to, the patient's ears, nose, nostrils, mouth, lips, chin, jaw, cheek, brow, or forehead. The light-therapy apparatus 20 can have a frame 22 that optionally engages with other features of a patient's head or portion of their anatomy. For example, the frame can engage with the crown of the patient's head, the top or back of the patient's head, the neck, or shoulders.

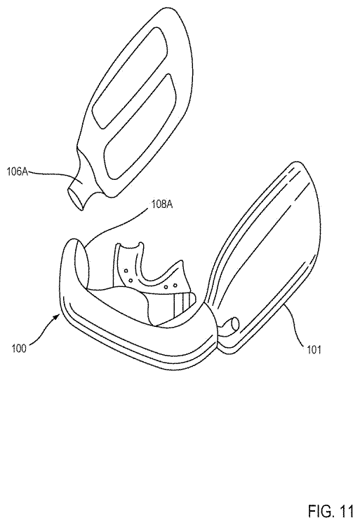

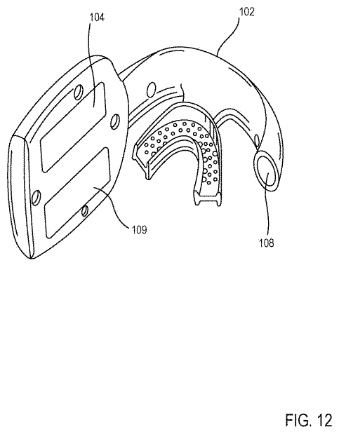

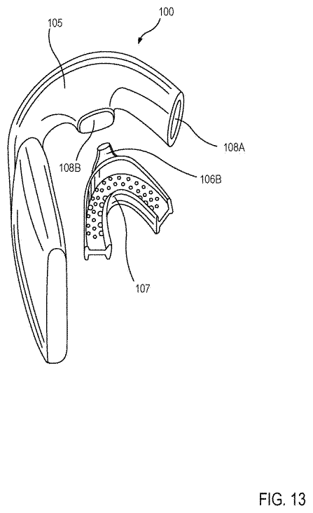

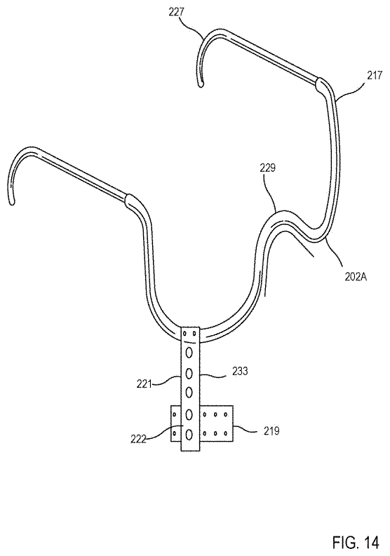

In the illustrative embodiment illustrated in FIGS. 14, frame 22 is shaped to provide ear-engaging portions 24, a nose-engaging portion 26, and support arms 28. A frame can engage with features of a patient's face by conforming to the shape of the feature, wrapping around the feature, overlying the feature, grasping the feature, adhering to the feature or providing pressure or weight to the feature. In some embodiments, frame 22 is formed as an integral unit. In other embodiments, frame 22 is formed from two or more separate pieces of material, which are suitably joined to provide frame 22. In some embodiments, frame 22 includes more than one type of material; for example, support arms 28 can be made from a material that is different from other portions of frame 22. Alternatively, the frame 22 can be formed of the same type of material.

Support arms 28 can be disposed so that they are overlying and contacting a patient's face, directly over the patient's jawbone when light-therapy apparatus 20 is worn in a use configuration by a patient. Portions 24 and 26 facilitate retention of light-therapy apparatus 20 on the facial area of a patient, while support arms 28 support a plurality of light sources 30 (also shown as light sources 30A-30H in some figures), as described herein. Support arms 28 can also facilitate engagement of light-therapy apparatus 20 on the facial region of a patient, e.g., by providing a biasing force inwardly against a patient's face. Other suitable configurations of frame 22 in addition to the illustrated embodiment are useful for securing light-therapy apparatus 20 to a patient's face and to support light sources 30 at the desired locations and with the desired orientations. The frame can support one or more light sources so that they contact the patient's face. The frame can be positioned so that the light source contacts the skin of a portion of the face overlying the region.

The frame 22 can include one or more support arms 28 that can be formed of an elongated portion. The support arms can be straight, curved, or bent in order to engage with a patient's face as desired. In some embodiments, the frame 22 includes other shaped portions that can include surfaces that can be flat, curved, or bent, that can cover one or more portion of the face. In one embodiment, the frame 22 can be curved over the bridge of a patient's nose, or curved around their cars. The frame can curve around the mouth or around a portion of the mouth.

FIG. 2 provides an example of a frame 22 where four elongated support arms extend around the mouth. For example, one, two or more support arms can be provided below the mouth. The support arms can be configured to lie over the patient's face, directly above the patient's jaw. One, two or more elongated support arms can be provided above the mouth or below the nose. The support arms can form two tracks, an upper track above the mouth, and a lower track below the mouth. In another embodiments, only one track is provided, which can be above the mouth, below the mouth, or in line with the mouth. Alternatively, additional tracks can be provided; for example, multiple support arm tracks can be provided above the mouth, below the mouth, or in line with the mouth. The support arms can lie over a right side or a left side of the patient's face. In some embodiments, an elongated support arm can form a continuous piece lying over both a right side and left side of a patient's face. Alternatively, separate elongate portions can be provided for a right side and left side of a patient's face. Elongate portions can optionally overlie a central region of the patient's face. In some embodiments, elongate portions do not overlie a central region of the patient's face. Any discussion herein of elongated support arms can also apply to support arms or other portions of the frame 22 that can have other shapes. Any arrangement of support arms can be applied to any of the light-therapy apparatus embodiments described herein.

In some embodiments, a support arm can include a support feature. In some embodiments, at least one of a right side of the support or left side of the support can comprise a support feature. In some embodiments, both the right and left side of the support can comprise support features. A support feature can allow one or more component of the light-therapy apparatus to removably engage with the support. In some embodiments, the support feature can allow the one or more components to move relative to the support while being engaged with the support. In some embodiments, the one or more components can comprise a light emitter, a light source, a secondary support, a hinge, or a light assembly. The support feature can be a track. In some embodiments, a track can include a slot, channel, groove, or other female feature which can be configured to accept a protrusion, ridge, or any other male feature, which can be provided on a component, such as a light source, a secondary support, a hinge, or a light assembly. In one embodiment, the track can be formed on an inner surface portion of the support (e.g., side of the support closer to a patient's face when in use). Alternatively, the track can be provided on an outer surface portion of the support (e.g., side of the support further from the patient's face when in use). In some embodiments, the track can be provided through the support. Alternatively, a support feature, such as a track, can have male features that can engage with a female feature of a component. Interlocking features can be provided between the support and one or more component.

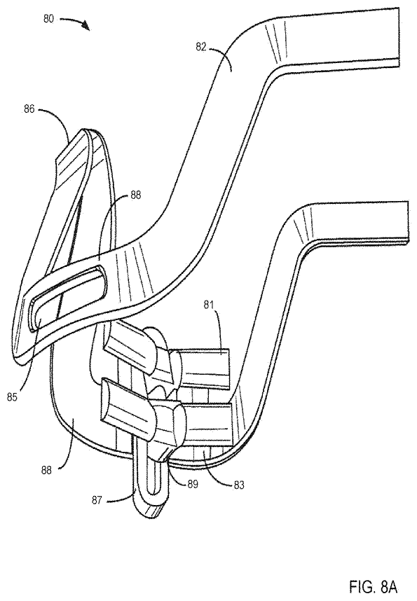

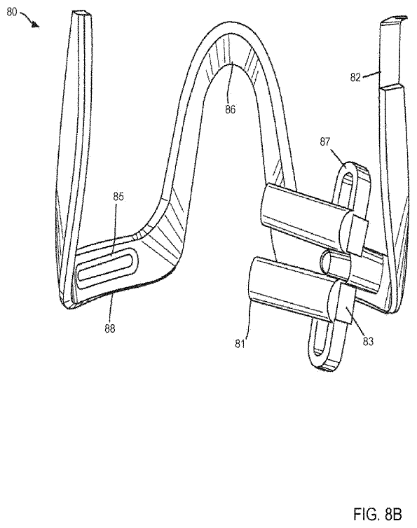

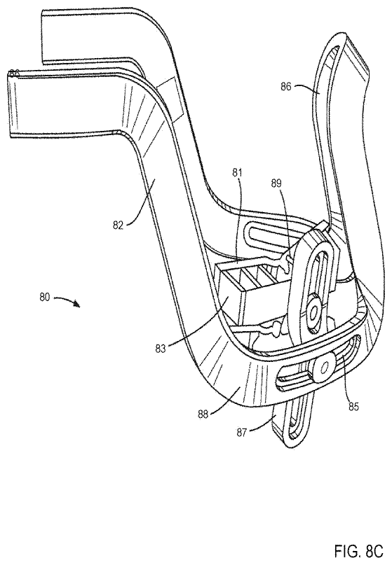

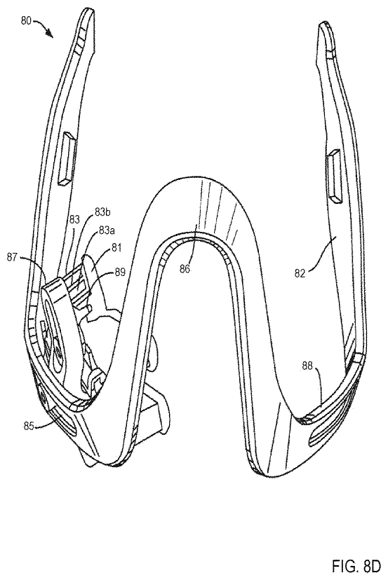

FIGS. 8A-8D show another embodiment of a light-therapy apparatus 80. The light-therapy apparatus 80 can have a frame 82 which is sized and/or shaped to engage with features of a patient's face. The frame 82 can optionally be shaped to engage with features of a patient's head or another portion of the patient's anatomy. Alternatively, the frame 82 is not shaped to engage with other features of the patient's head or other portions of the patient's anatomy.

In some embodiments, the frame 82 can be shaped to provide ear engaging portions, a nose engaging portion 86, and support arms 88. In some embodiments, the frame 82 can be formed as an integral unit. For example, the ear engaging portions, the nose engaging portion, and the support arms can be formed of a continuous integral unit. In one instance, the ear engaging portions, the nose engaging portion, and the support arms can form a single continuous elongated piece. In other embodiments, frame 82 can be formed from two or more separate pieces of material, which are suitably joined to provide frame 82. In some embodiments, one support arm per side of the face can be provided. Alternatively, multiple support arms per side of the face can be provided. One or more support arm can be engaged with the nose engaging portion or ear engaging portion.

Support arms 88 can be disposed so that they are adjacent to a patient's face overlying the jawbone or so that they are in the proximity of a patient's jawbone when light-therapy apparatus 80 is worn in a use configuration by a patient. In some embodiments, the support arms can be positioned so that one more light source 81 can contact the patient's face over the patient's jawbone or contact any other selected region of a patient's face. In some embodiments, the support arms can be configured to position one or more light source over one or more temporomandibular joint, condyle, or glenoid fossa of the patient. The light source can be positioned over a right temporomandibular joint, a left temporomandibular joint, a right condyle, a left condyle, a right glenoid fossa, or a left glenoid fossa of the patient. Portions, such as an ear engaging portion, nose engaging portion 26, or any other portion of a frame that can engage with features of a patient's face, can facilitate retention of light-therapy apparatus 80 on the facial area of a patient, while support arms 88 supports one or a plurality of light sources 81 (also shown as light sources 81A-81D in some figures), as described herein. Support arms 88 can also facilitate engagement of light-therapy apparatus 80 on the facial region of a patient, e.g., by providing a biasing force inwardly against a patient's face. Other suitable configurations of frame 82 in addition to the illustrated embodiment could be used to secure light-therapy apparatus 80 to a patient's face and to support light sources 81 at the desired locations and with the desired orientations. Other features, configurations, or components, as described in other embodiments, can be incorporated within this embodiment.

A frame, for any embodiment of a light-therapy apparatus, can be constructed from any suitable material; for example, lightweight plastic, steel, aluminum, copper, copper clad materials (such as aluminum or steel), nickel, titanium, silver, iron, other suitable metal or plastic, tubular plastic, plastic composite embedded with metal particles, graphite, graphite-epoxy, or any combinations or alloys thereof. The frame or portions of frame can optionally include a resin covering or suitable padding to cushion a patient's face. The frame can be made from flexible material, or from material which is thermally conductive. If a frame is made from a thermally conductive material such as, for example, aluminum, the frame can be capable of dissipating heat from one or more light sources, described below.

A frame can be made from a material which provides the frame with flexibility or which permits the frame to be conformed to the anatomical features of a particular patient's face. The frame or other components of the light-therapy apparatus can be bent in one or two dimensions. They can be moldable to conform to contours of the patient's face. A physician, dentist, orthodontist, therapist, technician or other individual, including a patient, can initially “fit” a particular light-therapy apparatus to a particular patient by adjusting and conforming that particular light-therapy apparatus to the anatomical features of that particular patient to provide an individualized fit. The material of which the frame is constructed can be sufficiently resilient to retain the individualized fit over the course of orthodontic therapy for that particular patient, and yet sufficiently flexible to permit that particular light-therapy apparatus to be re-adjusted (e.g. in response to complaints of discomfort from a patient) or adjusted to fit a different patient.

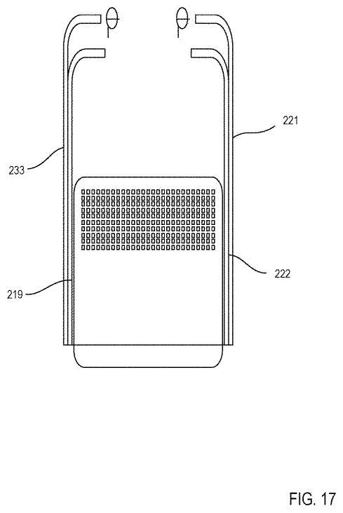

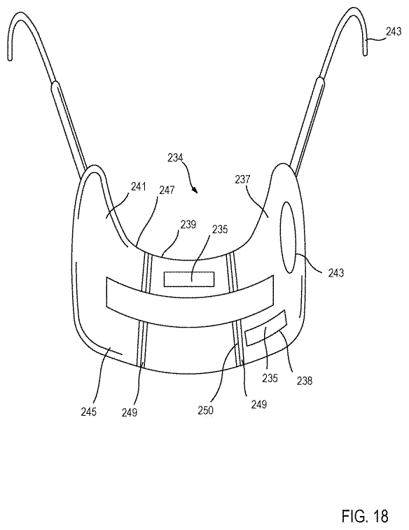

Any description, components, features, details of an embodiment of a light-therapy apparatus can be applied to any other embodiment of a light-therapy apparatus, and vice versa. For example, modifications to any device of FIGS. 1-4 (e.g., a frame 22 or light source 30 as provided in FIGS. 14) can be made to any of FIGS. 8A-8D (e.g., frame 82 or light source 81 in FIGS. 8A-8D), FIG. 9, FIG. 14, FIG. 17, or FIG. 18.

Providing a flexible frame 22 can also facilitate light source 30 contacting the cheek of a patient by support arms 28 (i.e., support arms 28 can bias light source 30 against the desired region of light administration on a patient's face, directly over his or her jawbone). In some embodiments, the morphology of the frame or the support arms, can cause the light source to contact a portion of a patient's face when the light-therapy apparatus is in use, e.g., when the light-therapy apparatus is worn by a patient. Other features can bias the light source, e.g., by providing pressure, to contact a portion of the patient's face, including but not limited to, elastic components, springs, inflatable portions, moving mechanical portions. Such bias can be provided when the patient's face is relaxed or when the patient's face is tensed. Bias of light source 30 on the cheek of a patient can depress the soft tissue, which can increase the effective transmission of light through the tissue. Thus, in some embodiments, it can be desirable for a light source to contact the skin of a patient's face or depress the skin of the patient's face.

In other embodiments, a gap can be provided between a light source and a skin of the patient's face. The frame can be configured to provide the gap between the light source and the patient's face. The light source can be in close proximity to the skin of the patient's face without contacting the patient's face. In some embodiments, the light source does not contact a patient's face when the patient's face is relaxed but can contact the face if the patient flexes a portion of the patient's face or tenses the face. In some embodiments, a light source can be about 1 mm or less, 2 mm or less, 3 mm or less, 5 mm or less, 7 mm or less, 1 cm or less, 1.5 cm or less, 2 cm or less, 2.5 cm or less, 3 cm or less, or any distance described herein, away from a patient's face while the patient's face is relaxed.

In some embodiments, the light source can contact a translucent or transparent material, such as a gel or solid film that contacts the patient's face. The frame can be configured so that the translucent or transparent material contacts the patient's face when the apparatus is in use. In some embodiments, the light source can include an exterior surface formed of a translucent or transparent material, such as a gel or solid film that contacts the patient's face. One or more light emitters of the light source can contact that exterior surface. Alternatively, a gap can be provided between the light emitters and the exterior surface. In some embodiments, the translucent or transparent material filters light of one or more particular wavelengths. In some other embodiments, the material dissipates heat generated by operation of the light source.

In some embodiments, a light emitter provided on a light source can be positioned at a distance from a region. The frame can be configured so that the light source is at a distance from the region. The region can be within a patient's oral cavity. In some embodiments, the light emitter can be provided external to the oral cavity. A portion of a patient's face, such as the cheek, lips, or chin can be lie between the light emitter and the oral cavity when the device is in use. A light emitter can be positioned at about 0.1 mm or less, about 0.5 mm or less, about 1 mm or less, about 2 mm or less, about 3 mm or less, about 5 mm or less, about 7 mm or less, about 1 cm or less, about 1.5 cm or less, about 2 cm or less, about 2.5 cm or less, about 3 cm or less, or any distance described herein, from a region.

Optionally, regions of greater flexibility than the remainder of frame can be provided between light sources or at other suitable locations on frame, to allow frame to be bent to provide a better fit around the facial area. Regions of greater flexibility can be provided, for example, by forming the region of greater flexibility from a portion of material that is thinner than the remainder of frame, by forming the region of greater flexibility from a material that is more flexible than the remainder of frame, or by providing hinge-like members (e.g., a thin crease or other bend line set into the material of which frame is constructed) within the frame. Other examples of how flexibility can be provided, can include using a bendable material, using a stretchable elastic material, using a spring, including multiple components that can slide or move relative to one another, that can unfold relative to one another, using telescoping features, including one or more joint (e.g., ball and socket, hinges), or having parts that can lock to one another at different size options. The frame can be adjustable to fit patients with different sized or shaped heads. In some embodiments, a frame size can be selected based on the size or shape of a patient's head.

In some embodiments, at least one light source 30 is secured to frame 22 in order to emit light towards a patient when light-therapy apparatus 20 is in the use position. Light source 30 is disposed extra-orally, i.e., outside of a patient's oral cavity, when light-therapy apparatus 20 is in the use position. When in use, the light source irradiates through the skin of a patient's face. Light can reach a region that is within a patient's oral cavity by transcutaneously irradiating through the skin. In some embodiments, when in use, light from a light source 30 is not configured to directly irradiate into the oral cavity, and reaches the oral cavity only through the skin. In one embodiment, light can reach a region only transdermally.

A light-therapy apparatus can have one or more light source capable of emitting light in the wavelengths described herein. The light provided by the light source is not necessarily visible light—any desired wavelength can be used. For example, light emitted by the light source can include infrared light or near-infrared light. The light source can also irradiate in the visible light region. For example, the light source can be configured to irradiate light falling within or ranging from about 400 nm to about 1200 nm. In particular embodiments, the light source can be configured to irradiate light falling within or ranging from about 500 to about 700, about 585 nm to about 665 nm, about 605 nm to about 630 nm, about 620 nm to about 680 nm, about 815 nm to about 895 nm, about 815 to about 895 nm, about 820 nm to about 890 nm, about 640 nm to about 680 nm, or about 740 nm to about 780 nm. In some embodiments, the wavelengths can fall within or range from about 605 nm to about 645 nm, or from about 835 nm to about 875 nm. In some embodiments, the wavelengths can fall within or range from about 615 nm to about 635 nm, or from about 845 nm to about 865 nm. In some embodiments, the wavelengths can be about 625 nm or about 855 nm. In some embodiments, a light source can be configured to emit light at one, two, or more of the light ranges described. In some embodiments, a light source does not emit light outside one, two, or more of the light ranges described. In other embodiments, light emitters can be configured to irradiate light having other wavelengths, as desired for a particular application. The light sources described herein can emit light at any of the wavelengths described herein.

In some embodiments a light source can be capable of emitting light at one, two, or more peak wavelengths of emission. A peak wavelength can be the wavelength at which the highest intensity of light is emitted. In some embodiments, light can be emitted at a range of wavelengths and the peak wavelength can be the wavelength with the highest intensity within the range. In some embodiments, a peak wavelength can be provided at about 620 nm, about 640 nm, about 650 nm, about 655 nm, about 660 nm, about 665 nm, about 670 nm, about 680 nm, about 690 nm, about 800 nm, about 820 nm, about 830 nm, about 835 nm, about 840 nm, about 845 nm, about 850 nm, about 860 nm, about 870 nm, or about 890 nm. The light sources described herein can emit light having any of the wavelength characteristics described herein.

A light source can be any suitable light source, which can include one, two, three, four, five, six, seven, eight, or more light emitters. In some embodiments, a light source comprises about 10 to about 15 emitters, about 15 to about 20 emitters, about 20 to about 30 emitters, about 30 to about 40 emitters, about 40 to about 50 emitters, about 50 to about 70 emitters, or about 70 emitters to about 100 emitters. For example, a light source can comprise a light-emitting diode (LED) (e.g., gallium arsenide (GaAs) LED, aluminium gallium arsenide (AlGaAs) LED, gallium arsenide phosphide (GaAsP) LED, aluminium gallium indium phosphide (AlGainP) LED, gallium(III) phosphide (GaP) LED, indium gallium nitride (InGaN) I gallium(III) nitride (GaN) LED, or aluminium gallium phosphide (AlGaP) LED), which can be present in an array; or a laser, for example a vertical cavity surface emitting laser (VCSEL) or other suitable light emitter such as an Indium-Gallium-Aluminum-Phosphide (InGaAlP) laser, a Gallium-Arsenic Phosphide/Gallium Phosphide (GaAsP/GaP) laser, or a Gallium-Aluminum-Arsenide/Gallium-Aluminum-Arsenide (GaAlAs/GaAs) laser. In one embodiment the light source comprises a plurality of lasers. A plurality of light emitters capable of emitting light at several different wavelengths can be used for light source 30. Alternatively, one or more light emitters capable of emitting light at the same wavelength can be used for the light source. One or more light emitters can be arranged on a light source in any manner. For example, a plurality of light emitters can be arranged in one or more rows or columns. The rows or columns can form an array, or a staggered set of rows or columns, concentric shapes. Light emitters can be provided from any commercially available source, and can include but are not limited to Optowell XH85 vcsel, ULM Vcsel, or Osram MID LED.

A light source 30 can be of any size and shape useful to irradiate through a patient's face a specified region of the patient's maxillary or mandibular alveolar bone. For example, in some embodiments, the light source 30 can have a height of about 9-10 mm along a vertical axis tangential to a patient's face, and a width in the range of about 15-18 mm along a horizontal axis tangential to a patient's face, as measured when light-therapy apparatus 20 is in the use configuration. One or more dimensions of a light source range from about 1-70 mm. In some embodiments, one or more dimensions of a light source range from about 1-3 mm, about 3-5 mm, about 5-7 mm, about 7-10 mm, about 10-15 mm, about 15-20 mm, about 20-25 mm, about 25-30 mm, about 30-35 mm, about 35-40 mm, about 40-50 mm, or about 50-60 mm.

A light source can have any shape, which can include, but is not limited to, a substantially rectangular shape, square shape, triangular shape, hexagonal shape, octagonal shape, trapezoidal shape, circular shape, elliptical shape, crescent shape, cylindrical shape or half-circle. A light source can have rounded or pointed corners. In some embodiments, the dimensions of a light source can be about the same as dimensions for a region area. In other embodiments, the dimensions of a light source can be greater than the dimensions of a region area. Alternatively, the dimensions of a light source can be less than the dimensions of the region area. The relative areas of a light source and region can depend on a parallel, convergence, or divergence angle at which light is emitted.

In some embodiments, each of the light sources within a light-therapy apparatus can be the same size or shape. In other embodiments, the light sources can have different sizes or shapes. Light source size or shape can be selected to administer a desired distribution of light to a region. A light source can have one type of light emitter. Alternatively, a light source can have two, three, four, five, or more different types of light emitters. Each light source can have a different light emitter or combination of light emitters, or can have the same light emitter or combination of light emitters. For example, each light source can have LEDs emitting light within the range of about 585 nm to about 665 nm, and LEDs emitting light within the range of about 815 nm to about 895 nm. In another embodiment, a first light source can have LEDs emitting from about 585 to about 665 nm, while a second light source can have LEDs emitting from about 815 to about 895 nm.

In some embodiments, one or more light source can include a substrate supporting the one or more light emitters. For example, one or more light source can comprise an array of light emitters mounted on a flexible sheet of material that will hold a shape when it is bent. The flexible material can advantageously comprise a metal sheet that can serve as a heat sink or thermal path to a heat sink. The flexible sheet can be molded to conform to the contours of a patient's face while the light-therapy apparatus is being fitted or is in use. The substrate can also include a cushioned material that can contact a patient's face without causing discomfort.

In some embodiments, light emitters of different characteristics (e.g., wavelength, intensity, pulsing, size), can be provided for a light source. In some embodiments, the different light emitters can be evenly interspersed within a light source. For example, light emitters of a first wavelength can be evenly interspersed within light emitters of a second wavelength. Alternatively, different light emitters can be localized. For example, light emitters of a first wavelength can be provided within a first region of a light source, and light emitters of a second wavelength can be provided within a second region of the light source.

A plurality of light sources 30 can be disposed on frame 22 to administer light of the desired wavelength substantially uniformly to desired regions of a patient's face, so as to irradiate, in one embodiment through the face, the patient's maxillary or mandibular bone, such as the maxillary or mandibular alveolar bone, one or more temporomandibular joint, one or more condyle, one or more glenoid fossa, or any other region as described elsewhere herein. Any number of light sources can be disposed on a frame. For example, one, two, three, four, five, six, seven, eight, nine, ten, eleven, twelve, thirteen, fourteen, fifteen, sixteen, seventeen, eighteen, nineteen, twenty, or more light sources can be provided for a light-therapy apparatus. The light sources can be distributed along any portion of the frame. In some embodiments, the same number of light sources can be provided on the right side and the left side of the frame. Alternatively, different numbers of light sources can be provided the right and left sides of the frame. One, two, three or more light sources can be positioned to administer light to a region. In some embodiments, the light administered by light sources to a particular region can be the same for each light source, or can vary.

One or more of the light sources can be removable. In some embodiments, all of the light sources are removable, while in other embodiments, one or more of the light sources are not removable. In some embodiments, none of the light sources are removable. Different types of light sources can be used to provide a desired light with a desired distribution to a region. For example, different light sources can be used for different applications, such as different stages of orthodontic or orthopedic treatment. For example, a first light source providing light at a first wavelength range can be used for one purpose, and a second light source providing light at a second wavelength range can be used for the same or for a different purpose. Or a first light source having a first size or shape can be used instead of or in conjunction with a second light source having a second size or shape. Additional light sources can be added or removed. Different light sources can be added or removed during the course of a treatment, such as an orthodontic treatment, bone remodeling treatment, or any of the other treatments disclosed herein, or during the course of preventing one or more abnormal conditions disclosed herein.

Each individual light source 30 can be separately configured or separately controllable, to provide light of a specified wavelength or intensity to a specific region of a patient's jawbone, or any other region for a desired period. In one embodiment the light is provided through the patient's face.

In some embodiments, one or more groups or subgroups of light sources can be separately configured or separately controllable, while all light sources belonging to the group or subgroup provide light of the same wavelength or intensity. In another embodiment, all light sources belonging to a light-therapy apparatus can be controlled together.

In some embodiments, a light-therapy apparatus can be configured to administer light to only some regions of the patient's maxillary or mandibular alveolar bone, if it is desired that teeth in other regions do not need to be moved (e.g. it can be desired to move only the upper teeth of a patient, or only the lower teeth, or to use certain teeth as an anchor when moving other teeth by administering no light to the anchor teeth). The light-therapy apparatus can also be capable of providing light of different wavelengths to different regions of the patient's maxillary or mandibular alveolar bone, if it is desired to differentially manipulate the movement of a patient's teeth, as described below. For example, light of a first wavelength can be administered to a first region and light of a second wavelength can be administered to a second region. The first and second wavelengths can include any wavelengths described elsewhere herein, such as about 585 nm to about 665 nm, and about 815 nm to about 895 nm, respectively.

In some embodiments, light can be administered to a region that can include a portion of tissue (e.g., bone tissue, or soft tissue) or other regions within the patient's oral cavity without being administered to other portions of the patient's oral cavity. In some embodiments, light can be administered to a region that can include a portion of tissue (e.g., bone tissue, or soft tissue) or other regions within the patient's oral cavity at a much greater intensity than it is administered to other portions of the patient's oral cavity. For example, 3×, 5×, 10×, 20×, 50×, or 100× greater intensity of light can be administered to a region, than another portion of the patient's oral cavity. In some embodiments, this is achieved by applying to the patient one or more intraoral or extra-oral light-translucent or light-opaque masks that shield from light one or more non-regions. In some embodiments, light reaching a region can have an intensity that is greater than a threshold value. In some embodiments, the threshold value can be at an intensity as described herein.

A patient can position light-therapy apparatus 20 herself or himself to accurately and repeatedly irradiate a desired location in the patient's dental and maxillofacial areas when light-therapy apparatus 20 is in a use position. Consistent positioning of light-therapy apparatus 20 during the course of a patient's treatment can make therapy more effective and repeatable, and ease of use of light-therapy apparatus 20 can facilitate patient compliance with a given treatment regimen.

In the embodiment illustrated in FIGS. 1-4, a plurality of light sources 30A, 30B, 30C, 30D, 30E, 30F, 30G, and 30H are disposed at symmetrical locations about frame 22. In other embodiments, a plurality of light sources 30 can be disposed asymmetrically about frame 22, the position of light sources 30 on frame 22 can be adjustable, or one or more than one light source 30 can be removable, to permit light-therapy apparatus 20 to be configured to administer, in one embodiment through the patient's face, light to a specific region or regions of a patient's maxillary or mandibular bone, such as specific regions of the patient's maxillary or mandibular alveolar bone, temporomandibular joint, condyle, or glenoid fossa. For example, each light source 30 can be configured to irradiate the bone surrounding a specific number of teeth, for example two or three teeth, at a specific location.

In use, light is emitted from an inner surface 32 of one or more light source 30 extra-orally towards a desired area. As used herein, the term “inner surface” refers to the surface of an element that is closest to the facial regions of a patient when light-therapy apparatus 20 is in the use position. Inner surface 32 can have rounded edges 33, as shown for example in FIGS. 7 A and 7B, and can include a clear resin window covering the light emitters, to provide greater comfort for a patient when light-therapy apparatus 20 is in the use position and when the light emitter's contact the patient's face.

Any suitable light emitter can be used for the one or more light source 30. In some embodiments, light is emitted by arrays of discrete LEDs. The LEDs can be arranged in any of a wide variety of patterns. For example, the LEDs can be arranged in staggered parallel rows to maximize the density of LEDs in the LED array. The LEDs can be arranged to achieve substantially uniform optical intensity over the light-emitting inner surface 32 of one or more light source 30. Alternatively, the LEDs can be clustered or distributed to provide varying optical intensities over an area of a light source. In some embodiments, each array can comprise 5 to about 20 LEDs or other light emitters. In some embodiments, each array can comprise about 20 to about 50 or more LEDs or other light emitters. In other embodiments, light from one or more light source 30 can be emitted by one or more than one VCSEL. A plurality of VCSELs can be disposed in an array on a light source 30. The VCSELs can be disposed in aligned or staggered parallel rows. In another embodiment, a combination of different types of light emitters, such as LEDs and VCSELs can be provided for the same light source.

A light-therapy apparatus can be configured to provide light with a desired light intensity. In one embodiment the average light intensity produced by a light source 30 is at least about 10 mW/cm². In other embodiments, the average light intensity produced by a light source is be about 1 mW/cm² or greater, about 3 mW/cm² or greater, about 5 mW/cm² or greater, about 7 mW/cm² or greater, about 12 mW/cm² or greater, about 15 mW/cm² or greater, about 20 mW/cm² or greater, about 30 mW/cm² or greater, about 50 mW/cm² or greater, about 75 mW/cm² or greater, about 100 mW/cm² or greater, about 200 mW/cm² or greater, about 500 mW/cm² or greater, or about 1 W/cm² or greater. In other embodiments, the average light intensity produced by a light source can be about 20 mW/cm² or less, about 30 mW/cm² or less, about 50 mW/cm² or less, about 75 mW/cm² or less, about 100 mW/cm² or less, about 200 mW/cm² or less, about 500 mW/cm² or less, about 1 W/cm² or less, or about 2 W/cm² or less. In some embodiments, a light source 30 has an average intensity that is, or can be adjusted to be, in the range of about 10 mW/cm² to about 60 mW/cm², or about 20 mW/cm² to about 60 mW/cm². In some embodiments, the output of light source 30 is pulsed. In such embodiments, the peak light intensity can be significantly higher than about 50 mW/cm². In other embodiments, the output of light is continuous. In some embodiments, the light intensity can vary over time in a cyclical or non-cyclical fashion. The light intensity can vary with or without pulsing. In some embodiments, the light intensity can vary with pulse width modulation. Any other light intensity described herein can be provided by the light-therapy apparatus.

The light emitters can be controllable so that the number of lights that are on or off at a given period can be individually controllable. For example, each light emitter can be on or off relative to other light emitters. This can be desirable when it is desirable to administer light to different regions. Thus, the light-therapy apparatus can alter the position of light being administered. In another embodiment, each light emitter can be on or off relative to other light emitters. For example, at some times, light emitters emitting in a first wavelength range can be on while light emitters emitting in a second wavelength range can be off, vice versa, or both types of light emitters can be on or off. Thus, the wavelength of light being administered can be varied. In some embodiments, the intensity of light being administered can be varied (e.g., by turning some light emitters on or off, or varying the intensity emitted by the light emitters). If the light emitters are pulsed, their duty cycle can be adjustable; e.g., light emitters can be capable of having a duty cycle of about 10%, about 20%, about 30%, about 40%, about 50%, about 60%, about 70%, about 80%, or about 90%. The light emitters can be capable of pulsing can occur with any frequency. For example, light emitters can be pulsed on the order of every picosecond, nanosecond, microsecond, millisecond, second, multiple seconds, or minutes. Light emitters can provide light with frequencies of about 1 mHz, about 10 mHz, about 50 mHz, about 100 mHz, about 500 mHz, about 1 Hz, about 2 Hz, about 5 Hz, about 10 Hz, about 15 Hz, about 20 Hz, about 25 Hz, about 30 Hz, about 35 Hz, about 40 Hz, about 50 Hz, about 70 Hz, about 100 Hz, about 200 Hz, about 500 Hz, or about 1 kHz. The light-therapy apparatus can be controllable so that any of the aforementioned characteristics of light emission (e.g., whether the light is on or off, continuous or pulsed, duty cycle, frequency, intensity, wavelength) can be varied or maintained in accordance with instructions from a controller.

The light-therapy apparatus can be capable of emitting light with varying intensities. Any ratio of intensities can be provided for light emitted at any of the wavelengths. For example, light emitted at a first wavelength can have about a 1.1×, 1.2×, 1.3×, 1.5×, 1.7×, 2.0×, 2.5×, 3.0×, 3.5×, 4.0×, 5.0×, 10×, 12×, 15×, 20×, 30×, 50×, 100× intensity compared to a light emitted at a second wavelength. In some embodiments, the same number of light emitters having a first set of characteristics and a second set of characteristics can be provided. In other embodiments, more light emitters having a first set of characteristics can be provided than light emitters having a second set of characteristics. For example, about 1.1×, 1.2×, 1.3×, 1.5×, 1.7×, 2.0×, 2.5×, 3.0×, 3.5×, 4.0×, 5.0×, 10×, 12×, 15×, 20×, 30×, 50×, 100× light emitters having the first set of characteristics can be provided as light emitters having the second set of characteristics.

One or more light source 30 can include optical elements such as one or more lenses or reflectors to focus and direct light from the light source 30 onto a selected area. Any type of optical lens or reflector can be used. For example, an optical lens can be used to collimate the light, diffuse the light, or focus the light. In some embodiments, one or more Fresnel lenses or telecentric lenses can be used. Any type of reflector can be used. A lens can be provided to cause light divergence, or light convergence. For example, one or more mirrors can be incorporated. The mirrors can be used to assist with scattering, redirecting, or focusing the light. Such optical elements can be suitably encapsulated in plastic or similar material, which can be transparent, translucent or opaque. The plastic or other encapsulating material can form an exterior surface of a light source. The light emitters or optical elements can be provided within an interior portion of the light source. Alternatively, encapsulating materials need not provided, and the optical elements or the light emitters can be provided as an exterior surface of a light source. In some embodiments, there can be a gap between a light emitter and an encapsulating material. A gap can exist between a light emitter and an exterior surface of the light source.

An exterior surface of a light source can contact a patient's face. For example, an encapsulating material for a light source can contact a patient's face. In other examples, optics, such as a lens optionally contacts the patient's face. In some embodiments, a light emitter can contact the face directly, while in other embodiments, the light emitter does not contact the face directly.

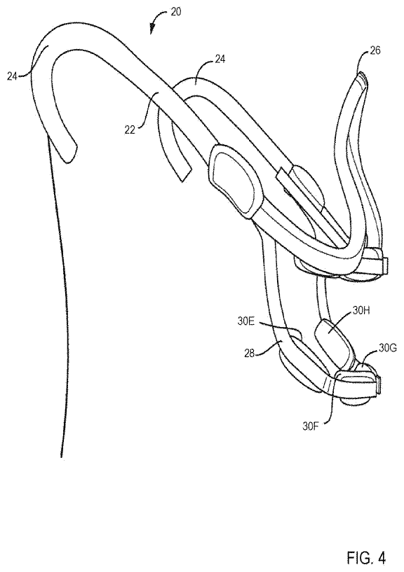

FIG. 5 shows a portion of a light source 30. In the illustrated embodiment, a light emitter 38 (which can, for example, comprise a junction in a light-emitting diode or other light-emitting semiconductor device) is located adjacent to a reflective backing 40. A curved light-reflecting recess 42 is provided adjacent to light emitter 38. Light from light emitter 38 is reflected in recess 42 to form a beam. The beams from all light emitters of a light source 30 can combine to irradiate the selected tissues. The area covered by the beam will depend upon the tissues which it is desired to treat. In some embodiments, the beam of light emitted by a light source 30 diverges to cover an area of tissue larger than the area of the light-emitting part of a light source 30. In other embodiments the emitted light converges to provide increased light intensity at the location of the tissues that it is desired to treat. In some embodiments, the emitted light diverges in a beam having an included angle θ in the range of about 45° to about 60°. The emitted light can form a diverging to have an included angle θ of 0° to about 15°, 0° to about 30°, 0° to about 45°, 0° to about 60°, 0° to about 75°, 0° to about 90°, or 0° to about 120°.

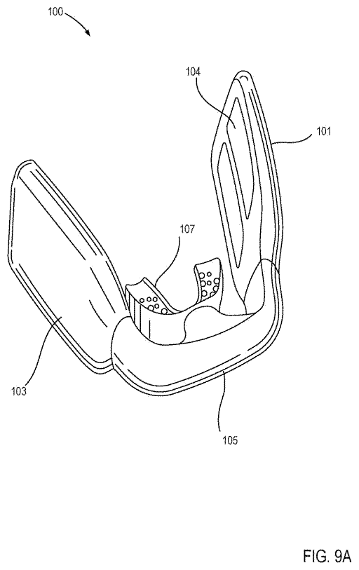

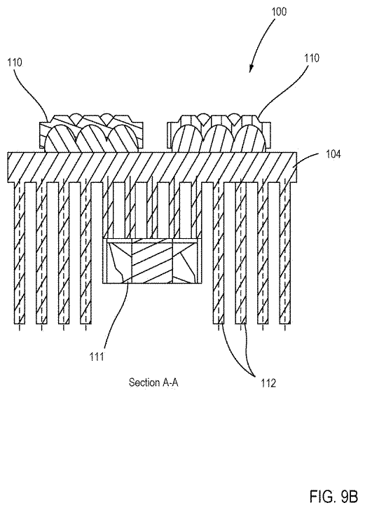

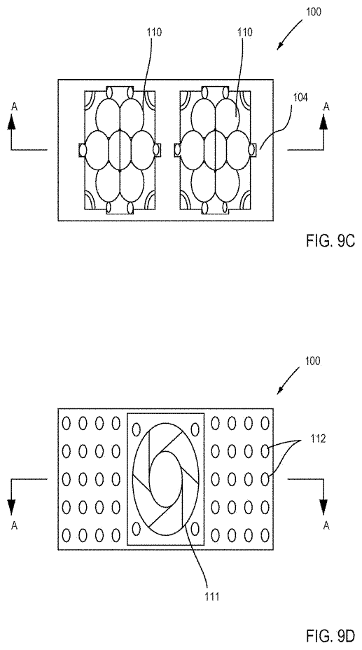

Since LEDs and other light emitters can emit heat when they are operated, it can be desirable to provide a suitable mechanism for dissipating the heat to prevent any parts of light-therapy apparatus 20 that are proximate to a patient's skin from getting too hot. In some embodiments, heat is dissipated by passive cooling, such as, for example, provision of appropriate heat sinks or permitting air to flow freely around light sources 30. Heat sinks 36 are an example of passive cooling. Heat sinks can be in thermal communication with one or more light source. In one embodiment, one or more light source can comprise thermally-conductive LED wafers mounted on a suitable heat sink. Heat from the LED wafers can be conducted into the heat sink and dissipated (see, e.g., FIGS. 9B, 9C and 9D as shown and as described below).

In some embodiments, one or more light source 30 can include a forced air, liquid, or solid state cooling system. In one embodiment, a heat sink has pins projecting from its face that is away from LED arrays. A fan causes air to flow past pins to carry away excess heat. Other fluids, such as other gases, or water or other liquids, can be driven past the pins to assist with carrying away excess heat.

A cooling system allows for administration of light without the danger of potential burns to the patient and allows for greater efficiency and control of the apparatus. A cooling system can be installed on light-therapy apparatus 20 in any suitable manner. The cooling system can be in thermal contact with one or more light source. In some embodiments, a cable recess (illustrated as 64A or 64B in FIGS. 7 A and 7B) can be provided within one or more light source 30 to accommodate aspects of a cooling system or cables that can be used with or form part of light-therapy apparatus 20.

In one embodiment, as shown in FIGS. 8A-8D, a cooling mechanism 83 can be provided. In one embodiment, the cooling mechanism can contact one or more light source 81, and can be formed of a conductive material. The cooling mechanism can conduct heat from the one or more light source and dissipate the heat to the surroundings. The cooling mechanism can function as a heat spreader or heat sink. The cooling mechanism can have an increased surface area by including one or more open region 83a disposed between one or more solid region 83b. A fluid is optionally forced through the cooling mechanism.

In one embodiment that can use either passive or active cooling, or both, support arms 28 can be constructed from milled aluminum, and one or more light source 30 can be constructed so as to be engageable with a track formed on the inner surface 34 of support arms 28, as shown for example in FIG. 7 A. One or more light source 30 can be engageable with a track 60 formed in the inner surface 34 of support arms 28 via a track-engaging ridge 62A formed on the one or more light source 30. Track 60 and track-engaging ridge 62A can have any suitable complementary configuration and orientation to retain one or more light source 30 against support arms 28 and oriented toward a wearer's face when light-therapy apparatus 20 is in the use position. One or more light source 30 can be slideable within track 60, to facilitate the positioning of light source 30. One or more light source 30 can alternatively be coupled to support arms 28 in any other suitable manner, such as by a clip, clamp, adhesive, thermally conductive adhesive, hook and loop fastener, or any other connection mechanism. In some embodiments, one or more light source 30 can be integrally formed with support arms 28.

In some embodiments, the track can have a fixed position relative to the rest of the frame. In one embodiment, a track can be a shaped feature within the frame. In other embodiments, the track can be adjustable to the rest of the frame. For example, the track can be formed of a material that can allow a user to bend the track to a desired configuration, and can stay at that configuration. In other embodiments, adjustment features, such as hinges, joints, or other moving parts can allow a user to adjust a track position.

One or more light source can slide along a length of the track. Alternatively, light sources can be attached or removed at different points along the track. In some embodiments, light sources can be attached or removed only at certain locations along the track (e.g., discrete portions that accept the light sources). Alternatively, one or more light source can be attached or removed at any point along the track. Thus, one or more light source can be displaced.

In some embodiments, one or more light sources can be applied to the frame so that they have a fixed orientation. Alternatively, the one or more light sources can be rotatable relative to the frame. Depending on the dimensions of a light source, this can allow variation in the region receiving light. One or more light source can be rotatable about one or more axis. For example, one or more light source can be rotatable about a first axis that is about parallel, i.e., ranging from +18° to −18° of being parallel, with the support arm, about a second axis that is perpendicular to the support arm, or about a third axis that is perpendicular to both the first and second axis. In some embodiments, one or more light source can be supported by a hinge, pivot, or other configuration that can allow one axis of rotation. In other embodiments, multiple hints, pivots, or other mechanisms can be provided that can allow for two or more axes of rotation. In another embodiment, one or more light source can be supported by a ball and socket joint that can provide multiple degrees of freedom. The orientation of one or more light source relative to the frame can be manually adjusted. A user can turn one or more light source to a desired orientation. Alternatively, the orientation of one or more light source can be remotely controlled. For example, one or more actuator can be provided that can cause one or more light source to turn to a desired orientation. Actuators can operate based on a signal received from a controller. The signal can be received via a wired connection or wirelessly, as described herein.

In another embodiment, as shown in FIGS. 8A-8D, one or more light source 81 can be configured to slide along a support arm 88. For example, a support arm on the right side of the face and a support arm on the left side of the face can include a track 85 that can enable a light assembly to slide along the track. The track can be parallel to the support arm. Alternatively, the track can be provided at some non-parallel angle to the support arm. In some embodiments, the track or support arm can have a substantially horizontal orientation when the apparatus is in use. A light assembly can include one or more light source 81, temperature control system 83 or vertical track 87. In some embodiments, one or more light assembly can be provided on a right support arm or one or more light assembly can be provided on a left support arm. In some embodiments, a support arm does not include a light assembly. A track on a support arm can be about horizontal, i.e., ranging from +18° to −18° of being horizontal. In alternate embodiments, the track can have any other orientation, which can include a vertical track, slanted travel, or curved track. In some embodiments, one, two, three, or more tracks can be provided on a support arm. The position of a light assembly relative to a support arm can be manually adjusted. For example, a user can push the light assembly to a desired position along the support arm. Alternatively, the position of the light assembly can be remotely controlled. For example, one or more actuator can be provided that can cause the light assembly to move to a desired position. Actuators can include, but are not limited to, motors, solenoids, linear actuators, pneumatic actuators, hydraulic actuators, electric actuators, piezoelectric actuators, or magnets. Actuators can cause the light assembly to move based on a signal received from a controller.

In some embodiments a vertical track 87 can be provided. The vertical track can be about perpendicular. i.e., ranging from +9° to −9° of being perpendicular, to a track along a support arm 88. Any description herein of the vehicle track can be applied to any other secondary track of any orientation that can be in communication with a track on a support arm. The vertical track can be adjustable relative to a track on the support arm. For example, the vertical track can slide along the track along the support arm. In some embodiments, the vertical track can be removable or attachable to the support arm, such as on the track along the support arm. In some embodiments, the vertical track can be attachable at one or more location along the support arm. Such locations can be discrete or continuous. One, two, three, four, or more vertical tracks can be attachable to the support arm simultaneously. The position of a vertical track relative to a support arm can be manually adjusted. For example, a user can push the vertical track to a desired position along the support arm. Alternatively, the position of the light assembly can be remotely controlled. For example, one or more actuator can be provided that can cause the light assembly to move to a desired position. The actuator can respond to a signal from a controller. The vertical track is optionally rotatable relative to the support arm. For example, the vertical track can be rotatable so that it is no longer vertically oriented, but can be horizontally oriented, or provided at a slant. The vertical track can be rotated manually. Alternatively, one or more actuator can be provided that can cause the vertical track to rotate to a desired position. The actuator can respond to one or more signal from a controller.

One or more light source 81 can be configured to slide along a vertical track. Alternatively, one or more light source can be attachable or removable from the vertical track at discrete or continuous locations. The position of one or more light source relative to a vertical track can be manually adjusted. For example, a user can push one or more light source to a desired position along the vertical track. Alternatively, the position of one or more light source can be remotely controlled. For example, one or more actuator can be provided that can cause one or more light source to move to a desired position. One or more light source can have a fixed orientation relative to the vertical track. Alternatively, it can be rotatable about a first axis, second axis, or third axis, such as those previously described. One or more light source can be manually oriented, or can have an actuator that orients the light source in response to a signal received from a controller. In one embodiment, one or more light source can be attached to a vertical bar 89 that can allow the light source to rotate about the bar within a limited range. This can allow the light source to have a desired position relative to a patient's face when in use. In one embodiment, two light sources can be provided along a vertical track. In alternate embodiments of the invention, the vertical track need not be perpendicular to a support arm and vertical. For example, a secondary track can be provided at any angle relative to the support arm (e.g., at about 15 degrees, about 30 degrees, about 45 degrees, about 60 degrees, about 75 degrees, or about 90 degrees relative to the support arm). In some embodiments, the secondary track can have a fixed orientation relative to the support arm. Alternatively, the secondary track can be rotatable relative to the support arm.

In some embodiments, one or more light source can rotate or move relative to the secondary track. For example, a hinge, pivot, ball and socket joint, or other type of mechanism can be provided that can allow one or more light source to rotate relative to the second track. In some embodiments, one or more light source can rotate within a limited range. In some embodiments, the relative position of one or more light source can be adjusted manually. For example, one or more light source can contact a patient's face and the position of the light source can conform to the contours of the patient's face. For example, the relative angle of the light source can conform to the patient's face. In other embodiments, one or more actuator can be provided to adjust the position of one or more light source. An actuator can operate in response to a signal received from a controller. In some embodiments, the position of one or more light source can be locked so that once a desired configuration for the light source has been set, it is not be adjusted manually. Alternatively, one or more light source can be responsive to force, so that a patient or other individual can be able to adjust the position of the light source.

In some embodiments, a third track, or fourth track can be provided. In one embodiment, a third track can be provided on a secondary track, or a fourth track can be provided on a third track. The support arm can comprise any number of tracks that provide various degrees of flexibility in the locations of one or more light source. In other embodiments, the support arm comprises one or more other components or configurations which can include but are not limited to bars, notches, slides, elastics, or holes.

A heat sink 36 can interpose one or more light source 30 and inner surface 34 of support arms 28. Heat sink 36 can, for example, be made of copper, aluminum, or other suitable thermally conductive material, to enhance dissipation of heat from light source 30. With reference to FIG. 7B, heat sink 36 can be engageable with track 60 formed in the inner surface 34 of support arms 28 via a track-engaging ridge 62B formed on heat sink 36. Track 60 and track-engaging ridge 62B can have any suitable complementary configuration and orientation to retain heat sink 36 against support arms 28, and to retain light source 30 oriented toward a wearer's face when light-therapy apparatus 20 is in the use position. Heat sink 36 can alternatively be coupled to support arms 28 in any suitable manner, rather than via engagement with track 60 through optional track-engaging ridge 62B. For example, heat sink 36 can be coupled to light source 30 by a clip, clamp, adhesive, thermally conductive adhesive, hook and loop fastener, or any other connection mechanism. In some embodiments, heat sink 36 can be integrally formed with either or both of light source 30 or support arms 28. In some embodiments, a heat sink can be coupled to each light source.

A gas, liquid, or solid state cooling system can be provided on support arms 28 to maintain light source 30 at a suitable temperature, or passive cooling means can be employed as previously described. In some embodiments, the temperature of the inner surface 32 of light source 30 can be maintained below a temperature of about 41° C., in one embodiment, from about 20° C. to about 35° C. A cable recess, illustrated for example as 64A or 64B (FIGS. 7 A and 7B) can be provided in light source 30 to accommodate cables for carrying electricity to light source 30 or components of a gas or liquid cooling system. An optional sensor or a controller 50 as described below can be provided, to automatically switch off any light source if the temperature of inner surface 32 or some other designated portion of that particular light source 30 exceeds a predetermined value.

The temperature of a light source can be varied or maintained to maintain or approach a desired temperature. For example, a cooling system can be used to reduce the temperature of a light source and prevent it from becoming too hot. In some situations, a temperature control system can be provided that can prevent a light source from being too cold or too hot. A desired temperature range can be preset. The desired temperature range can be fixed or adjustable. In some embodiments, a desired temperature range can range about ±10° C., about ±7° C., about ±5° C., or about ±3° C. of the ambient air temperature, or range about ±10° C., about ±7° C., about ±5° C., or about ±3° C. of the skin temperature of the user wearing the apparatus.

In some embodiments, light-therapy apparatus 20 is disposed and supported exclusively or substantially external to a mouth of a patient. A light-therapy apparatus which is supported exclusively or substantially external to a mouth of a patient can facilitate the use of that light-therapy apparatus optionally with one or more of a wide variety of intra-oral orthodontic devices. For example, orthodontic appliances, such as those disclosed herein, can be provided as intra-oral orthodontic devices and employed in the present apparatuses or methods. In other embodiments, a portion of light-therapy apparatus 20 can be disposed within a mouth of a patient, to assist in securing or positioning light-therapy apparatus 20 on a patient's face or head. For example, bite wings or an intra-oral tray which is supported in position by having a patient hold the intra-oral tray between her or his upper and lower teeth can be coupled to light-therapy apparatus 20 to assist in retaining or supporting the apparatus. An example of a suitable intra-oral tray is described in PCT publication numbers WO2009/000075 and WO 2006/087633, both of which are incorporated by reference herein in their entirety. In some embodiments, an intra-oral device can comprise one or more light sources or be capable of intra-orally administering light to a region. In some embodiments, light can be administered to a region intra-orally or extra-orally or both. In other embodiments, light is administered to a region only extra-orally, and is not administered to a region intra-orally. In some embodiments, light can only be administered to a region transdermally through the skin of the patient.

FIGS. 9A-9D and 10-13 show an illustrative light-therapy apparatus 100 that comprises an extra-oral light source 104 having a right side 101 and a left side 103 (as viewed from the front of the apparatus), an extra-oral bridge 105, and an intra-oral tray 107. Intra-oral tray 107 registers to a patient's teeth. A light source 104 is rigidly connected to intra-oral tray 107 by extra-oral bridge 105. Alternatively, some flexibility can be provided between the intra-oral tray and the extra-oral bridge. Therefore, a patient can position a light source 104 accurately and repeatedly to irradiate a desired location in the patient's dental or maxillofacial areas by inserting intra-oral tray 107 into his or her mouth and biting intra-oral tray 107 so that it registers to at least some of the patient's teeth. This stabilizes light-therapy apparatus 100 and positions a light source 104 at a desired position. The consistent alignment and targeting of light from the light source 104 during subsequent treatments makes the treatments more repeatable.

In the illustrated embodiment, extra-oral bridge 105 is removable from an extra-oral light source 104 and intra-oral tray 107. Providing a light-therapy apparatus 100 having major components that are detachably connectable to one another adds versatility. A design which permits the major components of the light-therapy apparatus to be disassembled and reassembled while preserving alignment of extra-oral light source 104 to intra-oral tray 107 has the advantage that the apparatus can be disassembled for storage or transportation and then used immediately after assembly. FIG. 11 shows light-therapy apparatus 100 with extra-oral light source left side 103 detached from extra-oral bridge 105.

Extra-oral bridge 105, extra-oral light source right side 101, and extra-oral light source left side 103 can be secured together via a suitable connector. For example, extra-oral bridge 105, the extra-oral light source right side 101, and the extra-oral light source left side 103 can be connected by inserting male connector portions 106A of the extra-oral light source right and left sides 101 and 103 into corresponding female connector portions 108A of extra-oral bridge 105 (see FIG. 11). Suitably, the suitable connector allows extra-oral light source right and left sides 101 and 103 to be detached from extra-oral bridge 105 for case of use and flexibility.

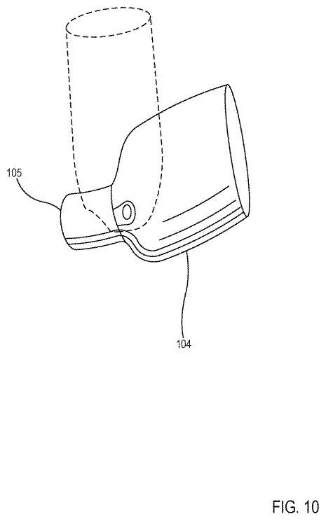

In some embodiments, extra-oral light source right and left sides 101 and 103 are rotatable between a sagittal orientation (as shown in FIG. 10) and a vertical orientation (indicated in dotted outline in FIG. 10). Light source right and left sides 101 and 103 can be locked at a desired angle of rotation by any suitable mechanism. This permits light source right and left sides 101 and 103 to be arranged so that the light that they emit fully covers the desired treatment areas.

Intra-oral tray 107 can be connected to extra-oral bridge 5 by way of another suitable connector. In the embodiment illustrated in FIG. 13, a male portion 106B of intra-oral tray 7 is removably received in a female portion 108B of extra-oral bridge 105. Where intra-oral tray 7 is removable from extra-oral bridge 105, extra-oral bridge 105 can be reused for other patients (after suitable sterilization). Intra-oral tray 107 can be disposed of after it is no longer required by a patient. In some embodiments, extra-oral bridge 105 is non-removably attached to intra-oral tray 107.

Intra-oral tray 107 can be inserted into a patient's mouth and can be suitably shaped to fit around a patient's teeth. Intra-oral tray 107 can register with a few selected teeth (for example, intra-oral tray 107 can comprise a bite tab) or can fit around the patient's full set of teeth. In one embodiment, the intra-oral tray 107 comprises a frame of a plastic or other suitable material that can serve as a skeleton for a settable material. The intra-oral tray frame can be perforated to aid retention of the settable material. The intra-oral tray frame can comprise extra-oral bridge 105 or a connector to connect to extra-oral bridge 105. The intra-oral tray can be optionally provided, and other securing means for an extra-oral bridge can be provided. For example, frames, as described elsewhere herein, can support an extra-oral bridge or extra-oral light source relative to the patient's face.

Prior to being used in the administration of light, a frame for intra-oral tray 107 can be filled with a suitable settable material (for example a clear vinyl siloxane gel or similar material) which sets around the patient's teeth and subsequently allows repeatable alignment of intra-oral tray 107 in the patient's mouth. Where intra-oral tray 107 could be in the path of light as it travels from light source 104 to selected tissues, the material of intra-oral tray 107 should be transparent to the light.

Extra-oral bridge 105 can conform around the jaw line of a patient. The light source right and left sides 101 and 103 can be respectively positioned on the right and left sides of a patient's face along the patient's jaw line. Extra-oral bridge 105 can be adjustable to permit alignment of light source left and right sides 101 and 103 with selected areas to be irradiated. Light source left and right sides 101 and 103 are extra-oral (outside of the patient's oral cavity). Light can pass from left and right sides 101 and 103 through tissues of the patient's lips and cheeks into selected areas on the patient's gums or in the patient's jaws. Light can be administered transcutaneously through the patient's face to any region as disclosed herein.

In some embodiments, one or more light source 104 emits light toward the patient. Any light source, with any configuration of light emitters as described anywhere else herein can be used. In some embodiments, a light source 104 has an inner surface 109 (see FIG. 12) that is placed near or against the patient's skin adjacent to the tissues that it is desired to treat. In some embodiments, one or more light source can contact the patient's face. The one or more light source can contact the portion of the face overlying a desired region. Light is emitted is from inner surface 109 toward the area of treatment. In some embodiments, left and right sides 101 and 103 of light source 4 each have a length similar to a significant fraction of the length of a human jaw. For example, left and right sides 1 and 3 can each have a length of about 20 mm to about 90 mm in some embodiments and about 25 to about 45 mm or about 60 mm in some embodiments. A light source can have any other dimensions, including those disclosed herein. In cases where a light source 104 is intended to treat or prevent a localized condition, then light source 104 can be smaller in extent. In some embodiments, light source 104 has optics that emit light in the form of diverging beams. The light source is usable with optics as described herein. In such cases, light source 104 can be somewhat smaller than the area of tissues to be treated because light from light source 104 can diverge as it passes through the tissues of the patient's lips and cheeks before reaching the tissues of the jaw and or gums.

Light source 104 can be wide enough to irradiate both upper and lower jaws of a patient simultaneously although in some embodiments light source 104 can be narrower. For example, light source 104 has a width in the range of about 12 mm to about 40 mm in some embodiments (e.g. about 15 to about 17 mm in some embodiments). In some embodiments, a light source irradiates only an upper jaw or a lower jaw, or portions thereof.

In some embodiments, the light source 104 includes thermally-conductive LED wafers mounted on a suitable heat sink. In use, heat from the LED wafers can be conducted into the heat sink and dissipated. For example, referring to FIGS. 9B, 9C and 9D, light source 104 can comprise one or more arrays 110 of LEDs that are mounted to a heat sink 104. Heat sink 104 can have pins 112 projecting or otherwise extended from its face and away from LED arrays 110. A fan 111 can be used to cause air to flow past pins 112 to carry away excess heat. In some embodiments, the light source can include any suitable mechanism described herein for dissipating the heat to prevent one or more portions of light-therapy apparatus 100 that are proximate to a patient's tissue from getting so hot as to burn the tissue or cause significant patient discomfort.

While the invention is described herein as usefully employing LEDs, other light emitters such as lasers could suitably be employed. The character of the light emitted by light source right and left sides 101 and 103 will depend upon the nature of the LEDs or other light emitters in light source 104. It is generally desirable that the emitted light include light in the wavelength range of 620 nm to 1000 nm. In some embodiments the emitted light includes light having a wavelength in at least one of the following wavelength ranges: about 820 to about 890 nm or about 620 to about 680 nm. In some embodiments, light having a wavelength in the ranges of about 820 to about 890 nm and about 620 to about 680 nm can be provided. Light having wavelengths corresponding to or falling within one or more of the following ranges can be particularly effective: about 613 nm to about 624 nm, about 667 nm to about 684 nm, about 750 nm to about 773 nm, about 812 nm to about 846 nm, or any other wavelengths described elsewhere herein. The range about 613 nm to about 624 nm corresponds to a band at which reduced cytochrome c oxidase absorbs light. The range about 812 nm to about 846 nm corresponds to a band at which oxidized cytochrome c oxidase absorbs light. Light sources can be configured to provide light of any other wavelength as described herein.

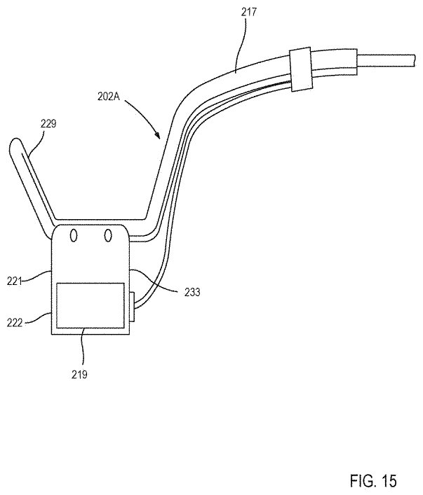

FIGS. 14 and 15 show a light-therapy apparatus 202A having a head-set style arrangement. Light-therapy apparatus 202A comprises a head-set 217 and at least one extra-oral light source 219 mounted to head-set 217 by way of a suitable connector 221. Head-set 217 can have the general form of a frame for eyeglasses. In the illustrated embodiment, headset 217 has arms 227 that fit above and around the patient's ears and a frame 229 that fits over the bridge of the patient's nose. Head-set 217 can also include lenses (not shown). Suitably, the lenses can be made of a material that blocks radiation at wavelengths emitted by light source 219 so that the patient's eyes are protected from the radiation. Light source 219 can comprise an array of LEDs or other light emitters.

When head-set 217 has been adjusted to fit an individual patient, frame 229 registers with the bridge of the patient's nose and arms 227 sit on the patient's ears. Head-set 217 is configured to sit on the patient's head in the same way each time it is put on. Head set 217 can be adjusted for fit by adjusting arms 227 which can be made of a firm, resilient material that allows for some flexibility for a better and more secure fit for individual users. In some embodiments, arms 227 can also be adjusted horizontally along their axis. Frame 229 can also be adjustable, for example, by bending to allow for a better and more secure fit. An elastic keeper such as an elastic strap can be provided to hold head-set 217 in place during use.

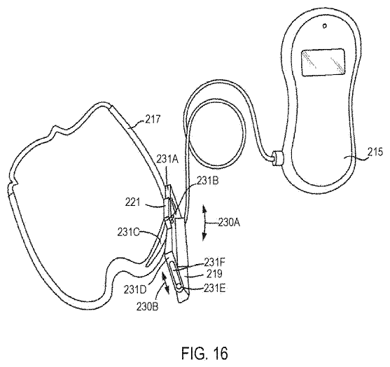

In the embodiment shown in FIG. 16, connector 221 permits the position of light source 219 to be adjusted both along a horizontal axis 230A and a vertical axis 230B relative to head-set 217. A yoke 231A is mounted to head-set 217 by screws 231B which pass through slot 231C. The position of light source 219 in horizontal direction 230A can be adjusted by loosening screws 231B, sliding yoke 231A to a desired position along slot 231C and retightening screws 231B. Light source 219 is connected to arms 231D of yoke 231A by screws 231E which pass through slots 231F. The vertical position of light source 219 can be adjusted by loosening screws 231E, sliding light source 219 up or down along slots 231F to a desired vertical position and then retightening screws 231E. Any other mechanism, including those described elsewhere herein, can be used to allow the light source position to be altered vertically or horizontally.

In the illustrated embodiment slot 231C is curved when viewed from above. Slot 231C generally follows the curvature of a typical maxillary bone such that light source 219 can effectively be applied against the patient's skin for a range of positions of light source 219 along slot 231C. Since the lower portions of people's faces are typically narrower than upper portions, connector 221 can hold light source 219 so that it is tilted with its lower edge projecting more in the direction of the patient than its upper edge. In some embodiments the angle of tile of light source 219 is adjustable. Head-set 217 can be adjusted so that light source 19 is biased against the patient's face when head set 217 is being worn by a patient. When the apparatus is in use, the light source can be contacting the patient's face. The light source can contact the region of the face overlying the region, thereby administering light transdermally to the region.

Many alternative designs for connector 221 can be provided. For example, connector 221 can comprise a bar, rod or similar device that can be clamped or otherwise fastened to head-set 217 and a clip or similar mechanism that fastens light source 219 to the bar, rod or similar device.

As shown in FIG. 17, in some embodiments light source 219 can be removably detached from headset 217. This can be convenient for storage or transportation of light-therapy apparatus 202A. When the apparatus is in use, the light source can contact a patient's face.

In another embodiment, head-set 217 comprises an adjustable strap (not shown) which fits around the crown of a patient's head for securing the extra-oral light-therapy apparatus 202A. The adjustable strap can also fit around a patient's chin and extend back to the crown and around the crown of a patient's head. The adjustable strap can be made of a flexible, elastic woven material.

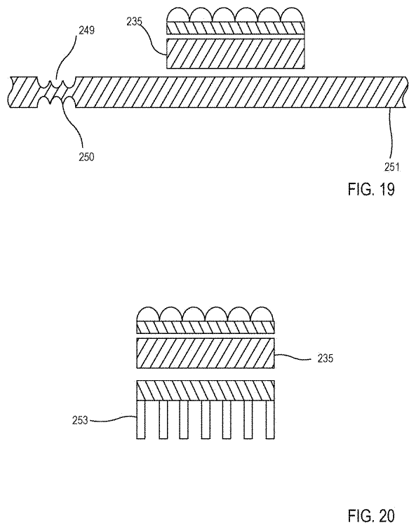

FIG. 18 shows a light-therapy apparatus 234 comprising at least one light source 235. Light source 235 comprises at least one light emitter, for example an LED array, mounted on a thin molded substrate 251 (FIG. 19). More than one array of light emitters can be provided in light source 235. For example, the light source 235 shown in FIG. 18 has two arrays of LEDs. Arrays 236 of light emitters can be arranged in lower level 245 and an upper level 247. The upper and lower levels can be separately controlled. The upper and lower levels respectively irradiate tissues of the upper and lower jaws. An attaching means 243 is provided for securing the apparatus to the area of treatment.

A power source and controller, which can comprise a programmable controller 215 as described herein, operate light source 235 to emit light according to a desired protocol. In some embodiments, the light-therapy apparatus can be removably coupled to the power source. For example, the light-therapy apparatus can be connected and disconnected to an external power source, such as a battery, using, for example, a cable, inductive coupling, or any other suitable means.

In the illustrative apparatus 234 shown in FIG. 18, light source 235 has a right section 237, a center section 239 and a left section 241. Right section 237 and the left section 241 are respectively supported on the right and left sides of a patient's face. One or more light sources can contact a patient's face when the apparatus is worn by the patient. A light source 235 as shown in FIG. 18 can be supported by way of any suitable attaching means including: a head-set 217 as described herein; an intra-oral tray 107 which can comprise a full tray or one or more bite tabs as described herein; an adhesive such as double-sided adhesive tape; a strap or set of straps; or supporting or attachment mechanisms.

The LED arrays can be removably attached to light source 235 by suitable connectors 238 such as ribbon connectors or can be more permanently integrated into light source 235 as illustrated in FIG. 19. Providing removable, repositionable LED arrays on a light source 235 permits LED arrays to be arranged on light source 235 so as to optimally irradiate selected tissues. LED arrays can be concentrated to irradiate selected tissues while areas of light source 35 that overlie non-selected tissues do not need to have any LED arrays.

FIG. 20 shows a cross-section of an LED array 236 of external light-therapy apparatus 234 detached from substrate 251. A clip or similar attaching means 253 allows the at least one LED array 236 to be mounted onto substrate 251. Substrate 251 can serve as a heat sink as described herein. Substrate 251 can be made of aluminum or similar metal that is a good heat conductor. Substrate 251 can be moldable (i.e., flexible in one or two dimensions so that it can be formed to follow contours of a patient's face and, once formed, retains its shape).

Hinge-like members 249 can be provided between arrays 236 to allow light source 235 to be bent to provide a better fit around the facial area. Hinge-like member 249 can comprise a thin crease 250 or other bend line set into the substrate material, as illustrated in FIG. 19. Hinge-like member 249 allows the center section 239 to fit around a patient's mouth and the right section 237 and the left section 241 to fit around a patient's face.

The apparatus can be applied by fitting a support to a patient. The support can comprise a head-set, intra-oral tray, a bite tab, one or more straps, one or more nose piece, one or more ear piece, or any other support or attachment mechanism. When the support has been fitted so that it can be repeatably worn by the patient one or more light sources can be attached to the support at locations where light from the light sources can irradiate a treatment area.

A treatment regimen can then be established. The physician, dentist, or therapist at her or his office or a patient at her or his home can optionally employ the apparatus in one or more methods of the invention.

Other embodiments, configurations, components, steps, or features can be incorporated in the invention. See, e.g., U.S. Patent Publication No. 2007/0248930 and U.S. Patent Publication No. 2006/0200212, which is hereby incorporated by reference in its entirety.

To calibrate the light-therapy apparatus, a sensor useful for measuring optical proximity (not shown) can be provided, positioned at a location that will be adjacent to, or substantially adjacent to, the skin of a patient (e.g., of an extra-oral light therapy apparatus) when the light-therapy apparatus is in the use position. The sensor can measure the optical proximity, for example, by measuring optical power reflected from the skin of the patient, with the sensor positioned in close proximity to, e.g., adjacent, one or more of the light therapy emitters. In some embodiments, the optical geometry (i.e., the position of the optoelectronic components, such as the sensor and the light therapy emitters) is selected such that the sensor's signal level declines, in some embodiments rapidly, with distance between the optoelectronic components and the skin of the patient. In some embodiments, if the value measured is outside of a predetermined range (e.g. because light-therapy apparatus has been displaced from a patient's head), the sensor can automatically pause a treatment or the emission of light from light source. Pausing treatment or the emission of light if light-therapy apparatus is displaced from a patient's head can minimize the risk of accidental injury, e.g., due to exposure of a patient's eyes to light from light source.

In some embodiments, depending on a signal from the optical proximity sensor, a controller can determine whether one or more light characteristic is to be maintained or adjusted (e.g., increased or decreased). Light characteristics can include, but are not limited to, light intensity, light wavelength, light coherency, light range, peak wavelength of emission, continuity, pulsing, duty cycle, frequency, duration, or whether a light emitter is on or off.

The light source can be configured to emit light that is substantially monochrome in some embodiments, although this is not mandatory. Providing light emitters that emit at multiple wavelengths allows for irradiation over multiple wavelengths for greater biological activity and greater selectivity and precision in administration. The light source can emit incoherent light, although this is not mandatory. In some examples, light can be provided at a single frequency, light can have a phase that drifts quickly, pulse of light waves can have an amplitude that changes quickly, or a light wave with a broad range of frequencies can be provided. The light can be administered continuously or pulsed at suitable frequencies and duty cycles. The light source can be configured to administer any of these light characteristics as described herein.

In some embodiments a light source emits light that includes infrared light, and the light source also emits light that includes visible light. The visible light, particularly visibly bright visible light, deters users from looking into light source 30 when it is operating, provides a perceptible indication that the apparatus is operating, and can be useful in properly positioning the light-therapy apparatus 20 described herein with reference to FIGS. 1-7B. The visible light can be, but is not necessarily, in a wavelength range that is beneficial for light therapy. In some embodiments, the ratio of the intensities of the visible and infrared components of the light is 1 part or less visible light to 5 parts or more infrared light. In some embodiments, a light source can comprise light emitters emitting light over a range of wavelengths. In some embodiments, the range can include wavelengths less than an order of magnitude. Alternatively, the range can include wavelengths emitted at one, two, three or more orders of magnitude.

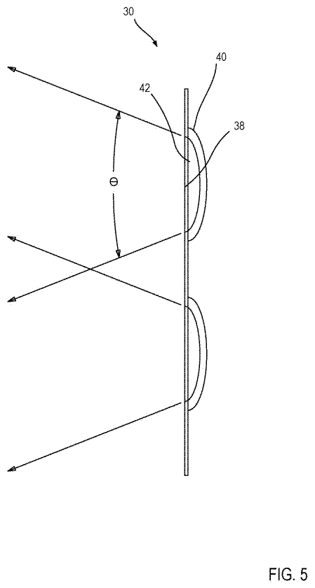

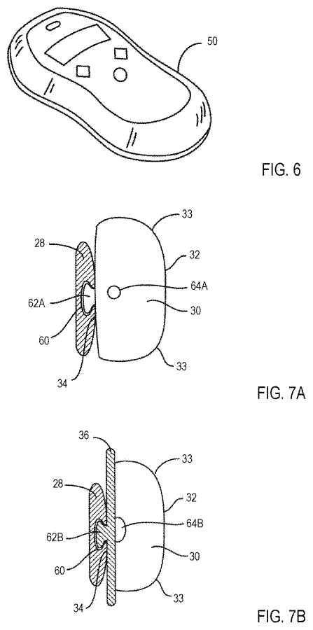

FIG. 6 illustrates an example of a programmable controller 50 of a type that can be used to control the operation of light-therapy apparatus 20. Although controller 50 is described in this illustrative embodiment as being programmable, it is not necessary that controller 50 be programmable. For example, a controller can have controls that allow various parameters to be set, such as light wavelength, light intensity, light pulsing, light duty cycle, light frequency, or light duration, and can appropriately activate light emitters of one or more light sources 30 in response to an appropriate signal. A controller can control light emissions with any light characteristics, which can include those described herein. Each of the light sources, e.g. light sources 30A-30H shown in FIG. 2, can be regulated independently by one or more controllers 50. A physician, dentist, orthodontist, therapist, technician or other professional can set those controls or program controller 50 so that an appropriate treatment is delivered when a patient initiates delivery of the treatment. Alternatively, the patient who is receiving the treatment might set controls. In some embodiments, the controls can include preset programs that can be suited to particular situations. In other embodiments, one or more parameter can be individually adjusted or entered.

In some embodiments, as shown in FIG. 6, a programmable controller can be a handheld device. Alternatively, the programmable controller can be part of another device or in communication with another device, such as a computer, which can include a personal computer, server computer, or laptop computer: personal digital assistants (PDAs) such as a Palm-based device or Windows CE device: phones such as cellular phones or location-aware portable phones (such as GPS); a roaming device, such as a network-connected roaming device; a wireless device such as a wireless email device or other device capable of communicating wireless with a computer network; or any other type of network device that can communicate over a network. Any discussion herein of computers or any other devices can apply to other devices, including controllers. A device can have a memory that can include tangible computer readable media that can include code, logic, instructions to perform any steps, calculations, algorithms, or execute programs or pre-stored instructions.

Programmable controller 50 can be a separate, remote unit or can be directly connected to or integrated with a light source 30. The programmable controller can connected to or integrated with any portion of the light-therapy apparatus, which can include a local controller, actuation mechanism, frame, or any other part of the controller.

A cable (not shown) can be provided to connect light-therapy apparatus 20 to programmable controller 50, a source of electricity for light source 30, or a suitable heating or cooling system. In some embodiments, wired communication can be provided between the programmable controller and the light-therapy apparatus. In other embodiments, the programmable controller and the light-therapy apparatus can communicate wirelessly. Examples of wireless signals can include, but are not limited to, radio-frequency (e.g., RFID) signals, bluetooth, or control-area-network (CAN) messages.

In some embodiments, controller 50 can comprise a microprocessor, data store, power supply, clock and associated electronic circuitry. A power source can include an external power source or an internal power source. For example, power can be provided by an electric plug. The plug might be in communication with a grid/utility, generator, or energy storage system. In some embodiments, the power source might be a renewable power source. The power source can be an energy storage system, such as a battery, ultracapacitor, or fuel cell. In some embodiments, the power source can be portable. In some embodiments, the power source as described herein can be separate from the controller 50, and the light-therapy apparatus and the power source can be configured to be releasably coupled to one another (e.g., via a power cable).

Control parameters are stored in the data store. A controller can comprise a memory that can include tangible computer readable media that can include code, logic, instructions to perform any steps, calculations, algorithms, or execute programs or pre-stored instructions. Programmable controller 50 operates light source 30 according to the parameters in the data store. The parameters can specify one or more of: treatment duration; wavelength or wavelengths of light emitted by light emitters 38; light intensity of particular wavelength or wavelength ranges during the treatment; whether light emitters 38 operate continuously or are pulsed; if light emitters 38 are pulsed, the rate at which light emitters 38 are pulsed: if light emitters 38 are pulsed, the duty cycle at which light emitters 38 are pulsed, light coherency of the light emitters 38, or any other light characteristic as described herein. The light emitters within the same light source can have the same light parameters. Alternatively, there can be light emitters of different light parameters within the same light source.

If light-therapy apparatus 20 has sets of light emitters 38 having different characteristics (e.g. sets of LEDs that emit light at different wavelengths or sets of light sources 30 that irradiate selected tissues in different locations) then separate control parameters can be provided for different sets of the light emitters 38 or light sources 30. In some embodiments, different sets of parameters are specified for different segments (intervals) of a light treatment. For example, light therapy treatments can be defined for a set of intervals each lasting from a few seconds to a few hundred seconds or a fraction of an hour. Different parameters can be specified for each of the intervals. The intervals are not necessarily equal in length. In some embodiments, a clock of a controller can assist in determining whether a predefined time interval has passed.

In some embodiments, different sets of parameters can be specified for different areas of light-therapy apparatus 20. In some cases, some light sources 30 of light-therapy apparatus 20 can be turned off because the treatment plan for a patient does not require light of particular wavelength or light at all wavelengths to be administered at locations corresponding to those parts of the light-therapy apparatus 20. For example, with reference to FIG. 2, programmable controller 50 can be programmed such that only light sources 30A, 30B, 30C and 30D are activated for a particular treatment regime in which it is desired that light therapy be administered only to a patient's upper teeth. Alternatively, programmable controller 50 can be programmed such that only light sources 30A, 30D, 30E and 30H are activated for a particular treatment regime in which it is desired that light be administered only to a patient's molars. Various other combinations and permutations of the activation of various light sources disposed about light-therapy apparatus 20 in any suitable configuration can be devised and implemented, depending on the desired application. In some embodiments, light-therapy apparatus 20 is configured (i.e. light sources 30 are positioned and oriented) so as to provide substantially uniform illumination of substantially the entire maxillary and mandibular alveolar bone or teeth of a patient. The light-therapy apparatus can be configured to provide substantially uniform illumination to other regions of the patient. The regions can optionally be limited to alveolar bone or basal bone.

A physician, dentist, orthodontist, therapist, assistant, technician, or other professional can program a patient's treatment regimen into programmable controller 50. This can be done, for example, with the aid of suitable software running on a computer that is in data communication with programmable controller 50 or by way of a suitable user interface built into programmable controller 50. In some embodiments, programming a treatment regimen can include specifying desired values for one or more parameter of light treatment. Programming a treatment regimen can also include specifying timing associated with the one or more parameters of light treatment. For example, a treatment regimen can be programmed so that for the first several minutes, light is provided at a first wavelength, and for the next several minutes, light is provided at a second wavelength. In some embodiments, default values can be provided. A user can be able to adjust the default values to create a customized light treatment regimen. In other embodiments, no default values are provided and a user can enter different parameter values.

Programmable controller 50 can have one or more pre-set programs built in. As an alternative to, or as an aid to programming controller 50, the physician, dentist, orthodontist, therapist or other professional can select a pre-set program that is appropriate for controlling light-therapy apparatus 20 to administer light to a patient. Such pre-set programs can be provided for particular types or stages of orthodontic treatment. In some embodiments, a pre-set program can be selected, and a user can modify the pre-set program as desired. For example, a user can be able to deviate from a pre-set program by adjusting any of the following: timing, light wavelength, light intensity, light pulsing or continuous, light duty cycle, light frequency, which lights are on or off, location of light source, or any other parameter that is described herein.

In some embodiments, a program can be determined prior to using the light-therapy apparatus. For example, after a user has created or selected a program, the light-therapy apparatus can be used, and one or more light source can emit light. In some embodiments, once a program is being implemented or a light-therapy apparatus is in use, the light treatment regimen is not be altered. In other embodiments, a light treatment regimen can be altered while the light-therapy apparatus is in use. For example, while light is being emitted, the light intensity can be adjusted, the light pulsing or continuous characteristics, the light wavelength, light selection, or location of the light source relative to a patient's face can be adjusted. The treatment regimen can be adjusted via the controller or a device in communication with the controller. In some embodiments, a patient wearing a light-therapy apparatus can adjust the treatment regimen. In other embodiments, physician, dentist, orthodontist, therapist, technician, assistant, or other professional can adjust the treatment regimen.

A user can interact with a user interface to program a controller, select a program or adjust a value of a program. Any user interface known in the art can be utilized. For example, a programmable controller can include one or more button, pointing device (e.g., mouse, joystick, trackball), keyboard, switch, knob, dial, touchscreen, or video display. The user interface can be provided to the controller directly, or can be provided to a device (e.g., computer) that can be in communication with the controller. A controller can include a display that can provide information to the user about selected parameters, timing or pre-set programs.

Programmable controller 50 can maintain a log of treatments that have been administered. For example, controller 50 can log the date and time that each treatment was initiated, the duration of the treatment, and whether or not the treatment was completed. The date and time can be logged based on a clock associated with the programmable controller. One or more timestamp can be provided indicating timing. The log can indicate parameters associated with the treatment. The log can be stored within a memory of the programmable controller. Alternatively, the log can be stored within a memory of a device in communication with the programmable controller, such as a computer.

The log can be accessed by a user to view log data. In one embodiment, the log can be accessed by a dentist, physician, orthodontist, technician, or patient who uses the light-therapy apparatus. A user can access the log directly from a controller or a device in communication with the controller. A user can access the log from any device that can be in communication with a device that stores the log data. The controller or devices can communicate directly with one another or over a network. The network can include a local area network, or a wide area network, such as the Internet.

This log can be subsequently reviewed by a dentist, physician, orthodontist or other medical professional to evaluate whether or not the patient has complied with a prescribed treatment regimen. The log can be displayed to a screen or other video display of a device. The log can track the times and durations of light therapy treatments administered by light-therapy apparatus 20 and can also track other features such as operating temperatures, operational status, treatment parameters, timing, or any combination thereof.

In some embodiments, a programmable controller 50 has a button or other suitable user patient interface that allows a patient to initiate a treatment according to previously-set parameters in the data store. In some embodiments, the patient interface is very simple such that minimal instruction is required to explain to a patient how to use light-therapy apparatus 20. Programmable controller 50 can include an audible or visual indicator that generates a signal to remind a patient that it is time for a treatment (or that a scheduled treatment is overdue).

In some embodiments, a treatment regimen can be pre-selected or programmed at the same device (e.g., controller, computer) through which a patient can initiate a treatment. Alternatively, a treatment regimen can be pre-selected or programmed at a different device (e.g., controller, computer) through which a patient can initiate a treatment. In some embodiments, communications can be provided between the controller and another device (e.g., computer) that can allow one or more treatment program to be delivered to the controller. In some embodiments, two-way communications can be provided between the controller and another device. In other embodiments, one-way communications can be provided from the other device to the controller or vice versa.

A patient can use light-therapy apparatus 20 at home or in another location by operating programmable controller 50 to initiate delivery of a treatment. The patient can use the light-therapy apparatus while at an appointment with a medical professional, or at a laboratory or clinic. Alternatively, a patient can use this apparatus while not at an appointment with a medical professional, or at a laboratory or clinic. The patient can use this apparatus while the patient is mobile or traveling.

Programmable controller 50 can comprise circuitry that monitors temperature at one or more locations in light source 30. The circuitry can monitor a signal modulated by a temperature sensor in light source 30. In some embodiments, the temperature sensor can be a thermocouple, thermistor, or resistance temperature sensor (RTD). In other embodiments, programmable controller 50 can monitor e.g. the current and voltage driving light emitters (e.g., LEDs, lasers) in light source 30. The current/voltage relationship can be temperature-dependent. Thus, by monitoring the current/voltage relationship programmable controller 50 can determine whether the light emitter (e.g., LED, laser) is at an undesirably high temperature. A temperature sensor can also be used to determine whether a light source or light assembly, or any component thereof is at an undesirably high temperature. Furthermore, the temperature sensor can determine whether a light emitter, light source, or light assembly has an undesirably low temperature. A temperature sensor can be used to determine whether any part of a light-therapy apparatus falls within a desired temperature range.

Programmable controller 50 can shut off or reduce current to any particular light source (e.g. one or more of light sources 30A-30H) when it detects that the temperature of that light source is undesirably high (or is trending towards being undesirably high). The programmable controller can also shut off or reduce current to any particular light emitter (e.g., one or more light emitter can be provided for a light source) if the controller detects that the temperature at that light emitter is undesirably high. Alternatively, the programmable controller can shut off or reduce current to a group or subgroups of light emitters or light sources if the temperature of a particular light emitter or light source is too high. For example, the programmable controller can shut off or reduce current to all light sources if a temperature is too high.

If light-therapy apparatus 20 is provided with a cooling apparatus, controller 50 can increase the operation of the cooling apparatus when it detects that the temperature of light source 30 is above a desired level. If increasing operation of the cooling apparatus does not bring the temperature of a light source or light emitter or any other portion of a light-therapy apparatus to a desired level, one or more light emitters or light sources can be shut off or reduced.

Shut-off or current reducing steps can occur automatically when a temperature threshold is reached. In some embodiments, a user can define the temperature threshold. In other embodiments the temperature threshold can be pre-set. In some embodiments, an alarm or alert can be provided when a temperature threshold is reached, and a user can manually shut off or reduce current to a light source or light emitter. In some embodiments, a temperature measurement can be displayed to a user.

FIG. 21A is a perspective view of a light-therapy apparatus in accordance with another embodiment of the invention. The light-therapy apparatus can optionally have one or more ear pieces 90 configured to fit around the patient's ear. The length of the ear pieces can be adjustable relative to the frame 82. In some embodiments, an ear switch 93 or mechanism can be used to allow the ear piece to adjust relative to the frame. In some embodiments, a vertical portion 91 of the frame can extend downwards from the frame. A support arm 88 can extend downwards from the vertical portion of the frame. In some embodiments, the support arm is adjustable relative to the vertical portion of the frame. The support arm can move up or down relative to the vertical portion of the frame. A support switch 92 or other mechanism can be used to allow the support arm to adjust relative to the vertical portion of the frame. A vertical hinge 89 can connect to a secondary support 96 that can slide along the support arm in a track 85. A screw 97 or other mechanical feature can be used to maintain or adjust the position of the secondary support relative to the track. The screw can be loosened to allow the secondary support to slide along the track 85 or tightened to keep the secondary support in place. In some embodiments, an upper vertical hinge 89a can be provided above the secondary support and a lower vertical hinge 89b can be provided below the secondary support. One or more light source 81 can be provided on the vertical hinge. In some embodiments, at least one light source is provided on the upper vertical hinge 89a and at least one light source is provided on the lower vertical hinge 89b. The light source can slide up and down the vertical hinge, or rotate on a vertical axis relative that is parallel to the vertical hinge. In some embodiments, a screw 98 or other mechanical feature can be used to maintain or adjust the position of the light source relative to the vertical hinge. The screw can be loosened to allow the light source to slide or rotate relative to the vertical hinge, or tightened to keep the light source in place. One or more wire 94 can be connected to a light source 81. The wire conveys signals to the light sources to control the light emitted from the light source. A wire 95 can connect the head set to a controller 52. The wire can provide electrical signals that can provide power to the light source, or instructions on when specific light sources should be on or off.

FIG. 21B shows a close up of an example of how a light source is supported in the light-therapy apparatus. A secondary support 96 can be positioned along a track 85 on a support arm 88. A screw 97 or other mechanical feature can allow the secondary support to maintain or adjust its position along the track. In some embodiments, a support arm can have one or more ridges 88a along the length of the track. The ridges can allow the secondary support to slide or snap into certain positions along the length of the track. One or more vertical hinge 89a, 89b can extend from the secondary support. In some embodiments, a vertical hinge can extend upwards 89a or can extend downwards 89b from the secondary support. Alternatively, a vertical hinge can extend upwards only, or downwards only. One or more grooves 99 or indentations can be provided on the vertical hinge. The grooves can provide positions for a light source to be affixed to the vertical hinge.

FIG. 22A shows an obverse view of a controller 52 in accordance with another embodiment of the invention. The controller can have a power button 53. The controller can have one or more display screen 54. The display screen can have one or more indicia 55. The indicia can be of time, battery level, wavelengths of light, settings, intensity, or any other information associated with the operation of a light therapy apparatus. FIG. 22B shows a reverse view of the controller. The controller can have a clip 56 that allows a patient to clip the controller onto an article of clothing. The controller can also have one or more wire connector 57 can connect to the light therapy apparatus or one or more power source.

Another aspect of the invention further provides for a light therapy kit comprising a light-therapy apparatus as described herein and instructions for use in the present methods. The kit can further comprise a light source that is separate from the light-therapy apparatus. The light sources can be disposable, so that they can be easily replaced after a given amount of use. In some embodiments, a light-therapy apparatus and light sources can be individually packaged or can be packaged together.

The kit can also comprise a programmable controller as described herein. The kit can further comprise any components useful for the controller to operate. For example, the kit can comprise a component to power the controller or light-therapy apparatus. The kit can also comprise a component that allows the controller to operably connect with a light-therapy apparatus.

The kit can also comprise software, an algorithm, or a set of computer readable media that can provide instructions to a controller. The software, algorithm, or set of computer readable media can be provided on a memory medium. The memory medium can be a removable or portable, such as a CD, USB flash drive, or external hard drive.

The kit can be conveniently packaged and can be commercially available. The kit can also include written instructions for use or maintenance of items therein.

In use, a physician, dentist, orthodontist, therapist or other professional can program a patient's prescribed treatment regimen into a programmable controller 50 (see FIG. 6, for example). Programmable controller 50 (or controller 52 in FIGS. 21 and 22) controls parameters of a light therapy treatment to be administered by light-therapy apparatus 20. For example, controller 50 can control the duration of the treatment, wavelength or wavelengths of light administered, light intensity, pulse frequency, or any other light or treatment characteristics. Programmable controller 50 runs a patient's prescribed treatment regimen causing the at least one light source 30 to emit pulsed or continuous light of specified wavelengths according to the prescribed parameters onto the treatment area of a patient's maxillary or mandibular alveolar bone. The treatment area can include any other regions described herein. This can include alveolar bone, basal bone, or teeth. Light can be administered mostly only to the treatment area. Light-therapy apparatus 20 can provide effective, stabilized repeatable, accurate, programmable, and consistent light therapy for a desired treatment to specifically administer light of a desired wavelength or wavelengths to a particular treatment region at a substantially uniform intensity. Scattering of light as it enters a patient's soft tissues can also cause the beam of light to diverge, resulting in uniform illumination of the patient's soft or hard tissue.

In accordance with another aspect of the invention, a light-therapy apparatus can be used in a method for administering light to a region of a patient's oral tissue. The methods can include providing a light-therapy apparatus comprising a support sized and/or shaped to engage with features of the patient's face and one or more light source supported by the support, engaging the support with one or more features of the patient's face, determining whether the position of one or more light source needs to be adjusted in order to administer a desired intensity of light to the region, depending on said determination, varying or maintaining the position of the one or more light source, and administering light to the region.

The light-therapy apparatus can optionally be an apparatus as described in any of the embodiments herein. The light-therapy apparatus can include a support that can be engaged with one or more features of the patient's face. For example, the light-therapy apparatus can engage with features of a patient's face by conforming to the shape of the feature, wrapping around the feature, overlying the feature, grasping the feature, adhering to the feature or providing pressure or weight to the feature. For example, the light-therapy apparatus can include an ear-engaging portion that can wrap around the back of the patient's ear. In another embodiment, the light-therapy apparatus can include a nose-engaging portion that can rest on the bridge of the patient's nose.

A method for administering light to a region can also include determining whether the position of one or more light source needs to be adjusted in order to administer a desired intensity of light to the region. Such determination can be made manually or automatically. For example, the patient or a medical professional can determine the position of a light source when the light-therapy apparatus is worn. The patient or medical professional can determine the relative position of the light source to a desired region. The light-therapy apparatus comprises one or more sensor. In some embodiments, the sensor can be a temperature sensor or an optical proximity sensor. In another embodiment, a sensor can determine the relative position of the light source with respect to the region. Determining whether a light characteristic needs to be adjusted in order to administer a desired light to the region can be based on one or more signal from the one or more sensor.

Depending on said determination, the position of the one or more light source can be varied or maintained. The position of the light can be varied manually or automatically. For example, a patient or medical professional can manually move a light source. In another embodiment, one or more actuator can be provided in communication with a controller. The controller can provide one or more signal to the actuator, thereby causing the actuator to move or maintain its position. The light source can be displaced, rotated, or tilted to provide a desired intensity of light to a region. In some embodiments, the light source can be pressed against the patient's face above the region, and the position of the light source can be set to that location. In some embodiments, after the position of a light source is adjusted, the light source can remain at that position in the absence of any outside force. In some embodiments, a light source can be locked into a position after it is adjusted, so that the light source can remain in that position even if a force is exerted on it.

In some embodiments, after a light has been set to a desired position, the method can include administering light to the region. In some other embodiments, light can be administered before or while the light is being set to a desired position. In some embodiments, a light-therapy apparatus can be engaged with the patient, the light source can be positioned, and the light can be administered without removing the light-therapy apparatus from the patient. In some embodiments, the light-therapy apparatus can be engaged with the patient, the light source can be positioned, and the light-therapy apparatus can be removed from the patient. This can be a series of steps for fitting the light-therapy apparatus to the patient. The light-therapy apparatus can subsequently be re-engaged with the patient and light can be administered to the patient. This can include steps for administering the light to the patient, after fitting the light-therapy apparatus to the patient. The light sources can already be positioned to administer light to the region. In some embodiments, light can be administered to the patient on multiple occasions following a single fitting.

In some embodiments, the method can include varying the position of one or more light source by adjusting the position of the light along the length of the support. The method the method can also include varying the position of one or more light by rotating the light source about an axis. The axis can be vertical, horizontal, or provided at any other orientation.

In some embodiments, light therapy apparatuses can be provided which are particularly suitable for intra-oral administration of light to one or more regions within a patient's oral cavity or mouth, such as a region of the patient's maxillary or mandibular alveolar bone. An intra-oral light therapy apparatus can incorporate one or more features or components of one or more embodiment of a light source or light therapy apparatus described herein. In one embodiment an intra-oral light therapy apparatus irradiates light having one or more characteristics of light described herein.

Examples of intra-oral light therapy devices can include a laser beam delivered by an optical fiber to a point of irradiation. In one embodiment, a low-energy laser source, such as a gallium-aluminum-arsenide laser can be used. See, e.g., Kawasaki, et al., “Effects of Low-Energy Laser Irradiation on Bone Remodeling During Experimental Tooth Movement in Rats,” Lasers in Surgery and Medicine 26:282-291 (2000); Cruz, et al., “Effects of Low-Intensity Laser Therapy on the Orthodontic Movement Velocity of Human Teeth: A Preliminary Study,” Lasers in Surgery and Medicine 35: 117-120 (2004); Abi-Ramia, et al., “Effects of LowLevel Laser Therapy and Orthodontic Tooth Movement on Dental Pulp in Rats,” Angle Orthodontist, 80(1): 116-122 (2010), which are hereby incorporated by reference in their entirety. Additional examples of intra-oral light emitting devices include U.S. Patent Publication No. 2007/0121786, U.S. Patent Publication No. 2008/0113313, U.S. Patent Publication No. 2009/0011380, U.S. Patent Publication No. 2009/0323370, which are hereby incorporated by reference in their entirety.

Other examples of intra-oral light therapy devices can include an oral tray that fits over one or more tooth or gums. In another embodiment, an oral tray need not fit over one or more tooth, but can be contoured to fit within a patient's oral cavity. Light from a light source can be transmitted to one or more teeth, or gum or mucosal tissue overlying one or more tooth root, via the oral tray. In some embodiments, the tray reflects or conveys light from a natural source (e.g., sun) or man-made source (e.g., lasers, LEDs, or light sources having any of the characteristics previously mentioned). In some embodiments, a light source is embedded within the tray or attached to the tray. In other embodiments, the intra-oral therapy devices include a cap-like structure that can fit over one or more tooth, or gum or mucosal tissue overlying one or more tooth root. The cap-light structure can transmit light from a distal light source. Alternatively, the cap-like structure comprises a light source provided therein. In some embodiments, the intra-oral light therapy devices are handheld devices that can provide or direct light to one or more tooth, or gum or mucosal tissue overlying one or more tooth root. The light can be provided from a proximal or distal light source. In some embodiments, the handheld devices comprise or otherwise utilize fiber optics. The light-providing portion of the handheld device can be held adjacent to a tooth, gums, or mucosal tissue overlying a tooth root. In some embodiments, the light providing portion of the handheld device can be located within a patient's oral cavity. See, e.g., U.S. Pat. No. 2,884,926: U.S. Patent Publication No. 2008/0255498; U.S. Patent Application No. 2006/0085052; U.S. Patent Publication No. 2008/0032252, which are hereby incorporated by reference in their entirety.

In some embodiments, a functional appliance and a light therapy apparatus are provided separately. Alternatively, a functional appliance can be integrally combined with a light therapy apparatus. A functional appliance-light therapy combination apparatus can have one or more removable components, or be integrally formed.

In some embodiments, a light therapy apparatus as described herein is useful for administering light intra-orally. Thus, a light therapy apparatus can be configured to provide light extra-orally or intra-orally or both. An intra-oral light therapy apparatus can be used in conjunction with an extra-oral light therapy apparatus as described herein.

In some embodiments, a light-therapy system comprises a light therapy apparatus and a vitamin D conveyance, configured to deliver an effective amount of vitamin D to the patient. In some embodiments, the vitamin D conveyance is attached to the support of the light-therapy apparatus. In some embodiments, the vitamin D conveyance contacts the patient's face when the light-therapy apparatus is worn by the patient. The vitamin D conveyance can be detachable from the support of the light-therapy apparatus. Alternatively, the vitamin D is not detachable from the light-therapy apparatus. In other embodiments, the vitamin D conveyance is separate from the light therapy apparatus.

The vitamin D conveyance can contain vitamin D. In some embodiments, the vitamin D conveyance contains vitamin D1, D2, D3, D4, D5, 1,25-dihydroxycholecalciferol, or mixtures thereof.

In some embodiments, the vitamin D conveyance is at least one of the following: a liquid, a transdermal gel, a patch, a cream, or a container comprising an injection pin or needle. The vitamin D conveyance can be configured to administer the vitamin D transdermally. The vitamin D conveyance can be configured to administer the vitamin D orally. The vitamin D conveyance can be configured to administer the vitamin D via injection. The vitamin D conveyance can be configured to administer the vitamin D via insolation.

In some embodiments, the vitamin D conveyance can selectively administer vitamin D to the patient. For example, the vitamin D conveyance can receive a signal in order to administer the vitamin D to the patient. In some embodiments, the vitamin D conveyance can receive a signal in order to stop administering the vitamin D to the patient. In some embodiments, the vitamin D conveyance only administers vitamin D while it is receiving a signal, or once it has received a signal. In some embodiments, the vitamin D conveyance can administer a fixed dosage amount of vitamin D to the patient. In some embodiments, the vitamin D conveyance can administer varying amounts of vitamin D, depending on a signal it receives. In some embodiments, the vitamin D conveyance administers varying amounts of vitamin D depending on a measured vitamin D level in the patient. In some embodiments, the signal is received from a controller. In some embodiments, the controller is local or remote to the vitamin D conveyance.

In some embodiments, the vitamin D conveyance automatically administers vitamin D to the patient. In some embodiments, the vitamin D conveyance automatically administers vitamin D to the patient while the conveyance contacts the patient. In some embodiments, the vitamin D conveyance automatically administers vitamin D to the patient while it contacts the patient's skin, such as the face.

In some embodiments, the vitamin D conveyance can allow vitamin D to be administered to the patient based on patient action or discretion. The patient can orally ingest a capsule, pill, liquid or other form of ingestible vitamin D.

In some embodiments, the vitamin D conveyance can be positioned over or contacting a region. The vitamin D conveyance can be positioned over or contacting oral or maxillofacial bone, muscle, or soft tissue, or one or more bone. The vitamin D conveyance can be positioned over the patient's mandibular bone, maxillary bone, temporal bone, or one or more teeth, or skin overlying the mandibular bone, maxillary bone, temporal bone, or one or more teeth.

The vitamin D conveyance can be attached to or incorporated into any of the light therapy apparatus components as previously described. In some embodiments, the vitamin D conveyance can be attached to or incorporated into one or more light source. For example, a light source can have a pad or patch that can convey vitamin D transdermally. A vitamin D cream, gel, ointment, or liquid can be on a pad. When the light source contacts the patient's face, the pad can also contact the patient's face. When the pad contacts the patient's face, vitamin D can be administered transdermally to the patient. In another example, one or more microneedles can be on a patch. When the light source contacts the patient's face, the patch can also contact the patient's face. When the patch contacts the patient's face, vitamin D can be administered transdermally to the patient. In another example, a light source can have one or more additional light emitters interspersed between the light emitters for administering phototherapy. The additional light emitters can be used to provide vitamin D via insolation. The additional light emitters can emit UV light.

In some embodiments, additional components can be provided to the light therapy apparatus. For example, the light therapy apparatus can have a vitamin D reservoir and a passageway fluidically connecting the reservoir to a target region. For example, a straw or tube can be provided that can deliver a liquid form of vitamin D to the patient's oral cavity. In another example, one or more microchannels can deliver vitamin D to a pad or patch that conveys vitamin D to the patient.

The vitamin D conveyance can be separate or separable from the light therapy apparatus components previously described. For example, a pad or patch that can convey vitamin D transdermally can be provided separately from the light therapy apparatus. A vitamin D cream, gel, ointment, or liquid vehicle can exist on be contained in a pad that contacts the patient's face. When the pad contacts the patient's face, vitamin D can be administered transdermally to the patient. In another example, one or more microneedles can be on a patch that contacts the patient's face. When the patch contacts the patient's face, vitamin D can be administered transdermally to the patient. In some embodiments, a UV light source can be provided separately from the light therapy apparatus. The UV light source can provide vitamin D via insolation.

In some embodiments, the vitamin D conveyance has a fixed location. During administration of vitamin D, the vitamin D conveyance can remain in the same location relative the patient's face. In some embodiments, the vitamin D can be at the same location relative to the patient's face whenever the light therapy apparatus is worn by the patient. In other embodiments, the vitamin D conveyance can have a variable location. In some embodiments, the vitamin D conveyance can remain at the same location relative to the patient's face during the vitamin D administration but can be moved before or after the administration of the vitamin D to another location. The vitamin D conveyance can move while the light therapy apparatus is worn by the patient. In other embodiments, the vitamin D conveyance can move relative to the patient's face during vitamin D administration.

Apparatuses and Methods for Intra-Oral Light Therapy

In one aspect, the invention provides an intra-oral apparatus, which includes a housing, an emitter and electronic circuit. The housing is configured to fit within a patient's mouth. The emitter is at least partially encased within the housing, and is configured to emit an effective amount of a light to a region associated with the alveolar soft tissue when the housing is disposed within the mouth. The electronic circuit is operatively coupled to the emitter, and is configured to control the emitter when the housing is disposed within the mouth and the apparatus is in use during orthodontic treatment.

The apparatus is useful for regulating tooth movement or for maintaining or improving oral tissue health. For example, the apparatuses described herein are useful in various embodiments in methods for regulating tooth movement. In one or more embodiments, the apparatuses are useful for treating patients with reduced periodontal bone support, for example, to increase bone volume. In one or more embodiments, the increase in bone volume is an increase in alveolar bone volume. In yet another embodiment, the apparatuses described herein are useful for hardening bone adjacent to bone roots, for example, in a patient who has undergone a bone graft surgery.

The apparatus described herein in one aspect, is useful in combination with an orthodontic appliance, such as brackets and wires, or one or more individual appliances, such as one or more incremental position adjustment appliances that effect incremental repositioning of individual teeth. Furthermore, in one or more embodiments, any light therapy apparatus shown and described herein is useful with any suitable orthodontic appliance, including, but not limited to, one or more incremental position adjustment appliances such as one or more substantially transparent aligners marketed under the trademark INVISALIGN™ (Align Technology, Inc., San Jose, Calif.). Such incremental position adjustment appliances are orthodontic appliances configured to move a patient's teeth and generally include one or more appliances (trays) wherein one or more of the appliances includes a shell having a cavity shaped to receive and resiliently reposition teeth from one tooth arrangement to a successive tooth arrangement. Each appliance of the set of appliances is in some embodiments worn by the patient in a predetermined sequence or order, and for a specified amount or period of time. In one or more embodiments, a patient undergoing light therapy with one of the light therapy apparatuses described herein together with incremental position adjustment appliance therapy undergoes the incremental position adjustment appliance therapy for about 8 to about 12 hours per day, as compared to about 20 to about 22 hours per day for a patient that is subjected to incremental position adjustment appliance therapy without light therapy. In another embodiment, the switching rate of the incremental position adjustment appliances for a patient undergoing light therapy with one of the light therapy apparatuses described herein, is accelerated as compared to a patient subjected to only incremental position adjustment appliance therapy (and no light therapy).

In one or more embodiments, the incremental position adjustment apparatus includes an aligner such as, for example, a transparent aligner such as INVISALIGN™, marketed by Align Technology, Inc., San Jose, Calif.

In one or more embodiments, the invention provides apparatuses that include a mouthpiece configured to fit within a patient's mouth. The mouthpiece includes a bite tray and a flange coupled to the bite tray. The flange spans from a first end of the bite tray to a second end of the bite tray. The flange is substantially rigid with respect to the bite tray, such that an angle formed between an inner face of the flange and an upper surface of the bite tray when the mouthpiece is disposed within the mouth is substantially unchanged, i.e., stays about the same angular value. The apparatus also includes light emitters disposed within the flange. The light emitters are configured to emit light to the patient's oral tissue when the mouthpiece is disposed within the mouth. In one or more embodiments, the light emitters are a single row of light emitters such as LEDs.

The invention further provides methods for regulating tooth movement, maintaining oral tissue health or improving oral tissue health. The methods include administering to a patient in need thereof an effective amount of light from the emitter of the apparatus.

In one or more embodiments, the invention further provides methods for orthodontic treatment that include disposing a mouthpiece of a light-therapy apparatus into a patient's mouth. The mouthpiece including a bite tray, a single flange, and a light emitter. The flange is coupled to the bite tray, the flange spanning from a first end of the bite tray to a second end of the bite tray. The light emitters are disposed within the flange. The flange is configured to be substantially rigid with respect to the bite tray during the disposing such that an angle between an inner face of the flange and an upper surface of the bite tray is substantially unchanged from before the disposing to after the disposing, i.e., stays about the same angular value. The disposing includes exerting a force, via the flange, on one or more teeth of the patient. The methods also include administering to the patient an effective amount of light from the light emitters. In one or more embodiments, the light emitters are a single row of light emitters such as LEDs.

The apparatuses are useful for increasing the rate of oral-tissue healing following oral surgery.

The invention further provides methods for increasing the rate of oral-tissue healing following oral surgery. The methods include administering to a patient's tissue on which oral surgery was performed and which is in need of healing an effective amount of light from the emitter of the apparatus.

In one or more embodiments, an apparatus includes a housing, an emitter and an electronic circuit. The housing is configured to fit within a patient's mouth. The emitter is optically coupled to the housing, and is configured to emit an effective amount of a light to the alveolar soft tissue when the housing is disposed within the mouth. The electronic circuit is operatively coupled to the emitter, and is configured to control the emitter when the housing is disposed within the mouth and the apparatus is in use during orthodontic treatment.

The apparatus is useful for regulating tooth movement or for maintaining or improving oral tissue health.

The invention further provides methods for regulating tooth movement, maintaining oral tissue health or improving oral tissue health. The methods include administering to a patient in need thereof an effective amount of light from the emitter of the apparatus.

The invention provides systems, including a first portion and a second portion. The first portion is configured to be disposed within a patient's mouth. A first emitter coupled to the first portion is configured to emit an effective amount of a light at a first wavelength to the alveolar soft tissue when the first portion is disposed within the mouth. An electronic circuit is operatively coupled to the first emitter, and is configured to control the first emitter when the first portion is disposed within the mouth and the apparatus is in use during a first stage of an orthodontic treatment. The first stage begins at a time TO. The second portion is different from the first portion, and is configured to be disposed within the patient's mouth. A second emitter is coupled to the second portion and is configured to emit an effective amount of a light at a second wavelength, different than the first wavelength, to the alveolar soft tissue when the second portion is disposed within the mouth. The electronic circuit is operatively coupled to the second emitter, the electronic circuit configured to control the second emitter when the second portion is disposed within the mouth and the apparatus is in use during a second stage of the orthodontic treatment. The second stage is subsequent to the first stage, and begins at a time T>0.

In one or more embodiments, the methods include receiving, each day for a predetermined number of days, an indication associated with contact between an orthodontic appliance and a tissue within an oral cavity of a patient. The orthodontic appliance is one of one or more orthodontic appliances that is removably coupled to the teeth of the patient. A treatment period associated with each orthodontic appliance from the plurality of orthodontic appliances is determined based on the indication. The methods further include producing a signal associated with the treatment period.

In one or more embodiments, the methods include disposing a first orthodontic appliance within an oral cavity of a patient such that the first orthodontic appliance is removably coupled to the teeth of the patient. A period specific to the patient is determined. The first orthodontic appliance is maintained within the oral cavity for the period. The methods further include disposing a second orthodontic appliance within the oral cavity of the patient after the period such that the second orthodontic appliance is removably coupled to the teeth of the patient.

The systems are useful for administering light therapy to alveolar soft tissue of a patient.

In one or more embodiments, an apparatus includes a mouthpiece configured to fit within a patient's mouth. The mouthpiece includes a bite tray and a flange coupled to the bite tray. The flange includes a top edge continuously spanning from a first end of the bite tray to a second end of the bite tray without a notch present therein. The flange is substantially rigid with respect to the bite tray. The apparatus also includes a light emitter disposed within the flange. The light emitter is configured to emit light to the patient's oral tissue when the mouthpiece is disposed within the mouth.

In one or more embodiments, an apparatus includes a mouthpiece configured to fit within a patient's mouth. The mouthpiece includes a bite tray and a flange coupled to the bite tray. The flange spans from a first end of the bite tray to a second end of the bite tray. The flange is substantially rigid with respect to the bite tray and has a height of about 1 cm, of about 1.2 cm, of about 1.5 cm, of about 1.8 cm, of about 2.1 cm, of about 2.4 cm, of about 2.7 cm or of about 3 cm, including all values, ranges and subranges in between. The apparatus also includes a light emitter disposed within the flange. The light emitter is configured to emit light, directly or indirectly, to the patient's oral tissue when the mouthpiece is disposed within the mouth.

In one or more embodiments, an apparatus includes a mouthpiece configured to fit within a patient's mouth. The mouthpiece includes a bite tray and a flange coupled to the bite tray. The flange spans from a first end of the bite tray to a second end of the bite tray. The flange is substantially rigid with respect to the bite tray. The apparatus also includes a single row of light emitters disposed within the flange. The row of light emitters spans from a first end of the flange proximal to the first end of the bite tray to a second end of the flange proximal to the second end of the bite tray. The row of light emitters is configured to emit light, directly or indirectly, to the patient's oral tissue when the mouthpiece is disposed within the mouth.

In one or more embodiments, a method for orthodontic treatment of a patient with reducted periodontal bone support includes disposing a mouthpiece of a light-therapy apparatus into a patient's mouth. The mouthpiece including a bite tray, a single flange, and a light emitter. The flange is coupled to the bite tray and spans from a first end of the bite tray to a second end of the bite tray. The disposing includes exerting a force, via the flange, on one or more teeth of the patient. The method also includes administering to the patient an effective amount of light from the light emitter.

Intra-Oral Light Treatment Apparatuses

One or more embodiments described herein relate to exposing the alveolar soft tissue, e.g., the alveolar mucosa, to light (e.g., having an intensity from about 10 to about 200 mW/cm²). Administering the light can modify the rate of tooth movement, increase the rate of healing, or provide one or more other orthodontic benefits. For example, administering light to an extraction site can increase the rate of healing and slow the movement of a tooth into the site. One or more embodiments described herein include an intra-oral light-therapy apparatus configured to administer light to one or more portions of the patient's alveolar soft tissue. Such an apparatus can be useful prior to, during or subsequent to orthodontic treatment and/or prior to, during or subsequent to oral surgery. In one or more embodiments, as disclosed herein, an apparatus can be used to administer light to a patient for 1 minute to 60 minutes per day. In other embodiments, the apparatus can contact a patient's oral mucosa for minutes, hours, day, weeks, months, or years, and one or more of the apparatus's emitters can irradiate light during at least some time during that period.

In one or more embodiments, an apparatus is configured to contact and/or be conformal with the alveolar soft tissue of any human patient, in other embodiments, an apparatus can be configured to be conformal with the alveolar soft tissue of a specific human patient. For example, the apparatus can be configured to be conformal with any human patient's, or to a specific human patient's, particular dental geometry, for example, using information obtained from CT scans (e.g., cone beam CT scans), models of the patient's jaw, intra-oral digital scanned models, and/or photographs of the patient's jaw. More specifically, the placement of LEDs within an apparatus to be positioned within the patient's mouth can be custom designed, using CAD/CAM design applications, for example, based on information obtained from one or more of the foregoing methods. In other embodiments, a standardized apparatus can be selected from one or more apparatuses configured to conform generally to human patients' oral anatomical features. In some such embodiments, the standardized apparatus can be adjusted to conform to a specific human patient's features.

In one or more embodiments, the apparatuses comprise a flange in which one or more light emitters are embedded, wherein the flange is configured to contact and/or be conformal with the alveolar soft tissue of any human patient; in other embodiments, the flange in which one or more light emitters are embedded is configured to be conformal with the alveolar soft tissue of a specific human patient. For example, the flange in which one or more light emitters are embedded can be configured to be conformal with any human patient's, or to a specific human patient's, particular dental geometry, for example, using information obtained from CT scans (e.g., cone beam CT scans), models of the patient's jaw, intra-oral digital scanned models, and/or photographs of the patient's jaw. More specifically, the placement of LEDs within a flange of an apparatus to be positioned within the patient's mouth can be custom designed, using CAD/CAM design applications, for example, based on information obtained from one or more of the foregoing methods.

As used herein, the phrases “conform[s] to” and “conformal with” refer to the property of an apparatus or its flange contacting, and adopting the same or substantially the same shape as or a complementary shape to, a surface of a wearer/patient of the apparatus. For example, when the light therapy apparatus is worn by a user, one or more flanges of the light therapy apparatus can contact the alveolar mucosa of the user, and deform to mimic or substantially mimic the shape (or complementary shape) of the alveolar mucosa of the user. The deformation can be facilitated, for example, by a material property of the material from which the one or more flanges are composed, and can include protruding in one or more regions thereof and/or recessing in one or more regions thereof. The light therapy apparatus can further be configured such that, when the light therapy apparatus is removed from the user's mouth, the one or more flanges of the light therapy apparatus return or substantially return to their original shape.

In one or more embodiments, an apparatus is configured to deflect, bend and/or deform to conform to the oral anatomy of a patient. For example, in one or more embodiments, an apparatus includes a mouthpiece configured to transition between a first configuration when the mouthpiece is outside of the patient's mouth and a second configuration when the mouthpiece is inside of the patient's mouth. Further, in one or more embodiments, an apparatus includes a mouthpiece and a light emitter. In one or more embodiments, the mouthpiece is configured to fit within a patient's mouth and includes a bite tray and a flange coupled to the bite tray. In one or more embodiments, an inner face of the flange forms a first angle with an upper surface of the bite tray, for example, when the mouthpiece is outside of the patient's mouth. In one or more embodiments the flange is deflectable with respect to the bite tray such that a second angle is formed between the inner face of the flange and the upper surface of the bite tray when the mouthpiece is disposed within the patient's mouth. In one or more embodiments, the light emitter is disposed within the flange and is configured to emit light to the patient's oral tissue when the mouthpiece is disposed within the mouth. In other embodiments, an apparatus is configured to be substantially rigid, and to resist deformation. For example, in one or more embodiments, an apparatus includes a mouthpiece configured to maintain its configuration when the mouthpiece is outside of the patient's mouth and when the mouthpiece is inside of the patient's mouth.

In one or more embodiments, a method for regulating tooth movement comprises disposing a mouthpiece of a light-therapy apparatus into a patient's mouth. In one or more embodiments, the mouthpiece includes one or more of a bite tray, a flange, and a light emitter. In one or more embodiments, the flange is coupled to the bite tray and optionally includes the light emitter therein. In one or more embodiments, the flange is configured to move with respect to the bite tray during the disposing such that an angle between an inner face of the flange and an upper surface of the bite tray has a first value before the disposing and a second value after the disposing. In one or more embodiments, the method further comprises administering to the patient in need thereof an effective amount of light from the light emitter.

In one or more embodiments, the apparatus is configured to administer light therapy based on a customized dosage, e.g., a dosage that is customized for a particular patient. Younger patients can have less dense bone than older patients. Density of the patient's bone can be measured, for example, using computed tomography (CT), in one or more embodiments, cone beam CT, prior to light therapy administration. In one or more embodiments, the patient's bone density can be measured by irradiating the patient's teeth and measuring the amount of light that penetrates the teeth (e.g., using an apparatus similar to or such as that depicted in FIG. 18A.) Once the patient's bone density is determined, an optimal dosage of light can be determined for achieving the desired tooth movement.

In one or more embodiments, the apparatus can include a bite pad to improve patient comfort when the apparatus is in contact with the patient's alveolar soft tissue and/or for positioning of the apparatus in a patient's mouth.

In one or more embodiments, the apparatus is useful in combination with an appliance that exerts a force on the patient's teeth and/or on muscular tissue such as buccal and labial cheeks, tongue, etc. In one or more embodiments, the apparatus of the invention is useful in combination with more than one appliance that exerts a force on one or more teeth of the patient of about 1 g, about 5 g, about 10 g, about 50 g, about 100 g, about 200 g, about 300 g, about 400 g, about 500 g, about 600 g, about 700 g, about 800 g, about 900 g, about 1000 g, about 1100 g or about 1200 g, including all values, ranges and subranges in between. Exerting one or more forces to the gum region, including on one or more of the patient's teeth, and intra-orally administering light to a patient's alveolar soft tissue can increase the rate of tooth movement, increase the rate of healing of oral tissue and provide other orthodontic benefits. In one or more embodiments, one or more of the forces exerted is a heavy force. In one or more embodiments, one or more of the appliances exerting a force is a functional appliance. Heavy forces and functional appliances are disclosed herein.

In one or more embodiments, the appliance, such as an incremental position adjustment appliance as disclosed herein is made from a material that enables the appliance to conform to the shape of the patient's teeth and/or other oral tissue. Examples of suitable materials include, but are not limited to, polymers such as polyurethane, silicone (including soft silicone), thermoplastics, and/or the like.

In other embodiments, the apparatus is useful in combination with an orthodontic appliance, such as, but not limited to, an aligner.

In one or more embodiments, a method for orthodontic treatment comprises disposing a mouthpiece of a light-therapy apparatus into a patient's mouth. The mouthpiece includes a bite tray, a flange, and a light emitter. In one or more embodiments, the flange is coupled to the bite tray and optionally includes the light emitter therein. In one or more embodiments, the flange is configured to move with respect to the bite tray during the disposing such that an angle between an inner face of the flange and an upper surface of the bite tray has a first value before the disposing and a second value after the disposing. In other embodiments, the flange is configured to be substantially rigid with respect to the bite tray during the disposing such that an angle between an inner face of the flange and an upper surface of the bite tray is substantially unchanged i.e., stays about the same angular value, from before the disposing to after the disposing. In one or more embodiments, the method further comprises administering to the patient an effective amount of light from the light emitter.

In one or more embodiments, the apparatus is useful in combination with an appliance or other suitable conveyance that is configured to deliver vitamin D to the patient. Vitamin D treatment raises the vitamin D blood serum levels of the patient and, when combined with intra-oral light treatment, can increase the rate of tooth movement, increase the rate of healing of oral tissue and provide other orthodontic benefits. Vitamin D treatment is disclosed herein.

In one or more embodiments, an apparatus of the invention is useful in combination with one or more appliances that exert a force on the patient's oral tissue, such as a patient's tooth, or with an appliance (or other suitable conveyance) that is configured to deliver vitamin D to the patient. In this manner, the patient receives light treatment and vitamin D treatment and also has forces exerted, for example, on one or more teeth.

In one or more embodiments, an apparatus is configured to detect and/or send a signal when a mouthpiece is disposed within a patient's mouth. In this manner, the apparatus can initiate and/or control delivery of any of the methods for light therapy disclosed herein. For example, in one or more embodiments, a light-therapy apparatus includes one or more of a mouthpiece, a bill and an electronics assembly. In one or more embodiments, the mouthpiece is configured to be disposed within a mouth of a patient, and includes a series of light emitters therein. In one or more embodiments, the series of light emitters is a single row of light emitters, such as, for example, a single row of LEDs. In one or more embodiments, the series of light emitters is configured to emit light to alveolar soft tissue of the patient. In one or more embodiments, the bill is coupled to an anterior end of the mouthpiece, and is configured to be disposed externally to the mouth when the mouthpiece is disposed within the mouth. In one or more embodiments, the least a portion of the electronics assembly is disposed within the bill. In one or more embodiments, the electronics assembly is configured to control operation of the light emitters. In one or more embodiments, the electronics assembly is further configured to send a first signal to cause a first light emitter to emit light. In one or more embodiments, the electronics assembly is configured to receive a second signal from a second light emitter, the second signal associated with the light emitted from the first light emitter. The electronics assembly is configured to detect when the mouthpiece is disposed within the mouth based on the second signal. In some embodiments, the electronics assembly is self-contained. Alternatively or in addition, in some embodiments, the electronics assembly is one or more of: hermetically sealed, waterproof, or water-resistant.

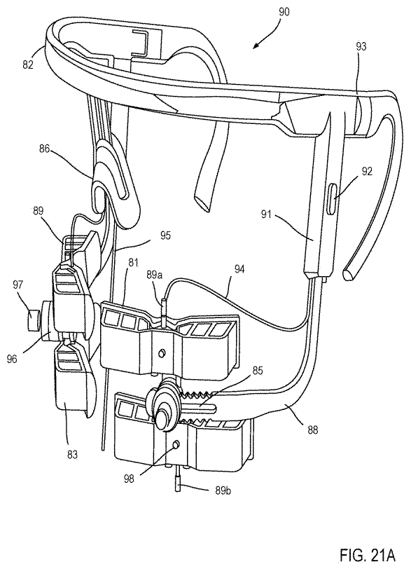

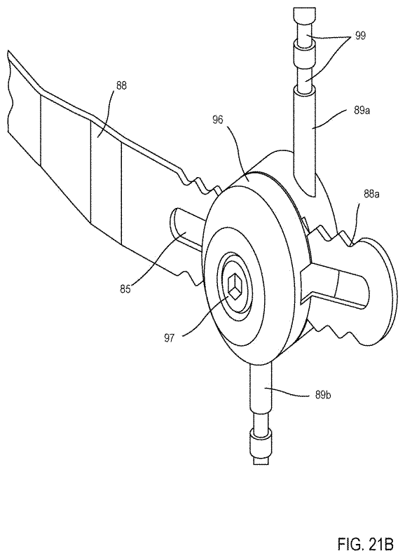

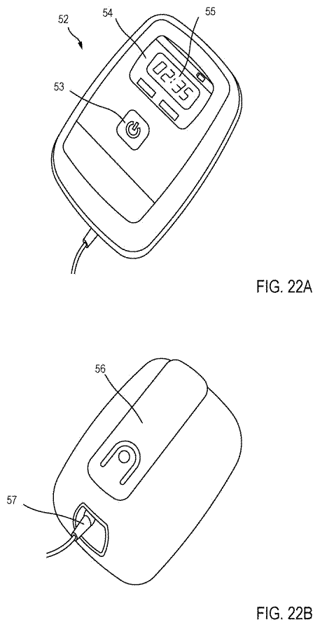

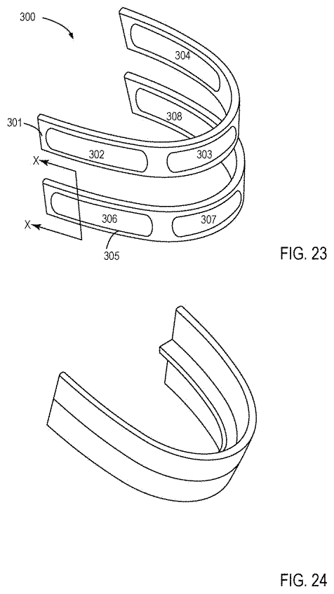

FIGS. 23 and 24 are schematic diagrams of embodiments of intra-oral light-therapy apparatuses of the invention. As shown in FIG. 23, an apparatus 300 includes an upper panel 301 having subpanels 302, 303, and 304, and a lower panel 305 having subpanels 306, 307, and 308. Panels 302, 303, and 304 are configured to be disposed near the root area of one or more teeth of the upper jaw. In one or more embodiments, subpanels 302, 303, and 304 can be configured to be disposed adjacent to the upper buccal alveolar soft tissue. For example, in one or more embodiments, the subpanels 302, 303, and 304 are in contact with the upper buccal alveolar soft tissue; whereas, in other embodiments, the subpanels 302, 303, and 304 are not in contact with the upper buccal alveolar soft tissue but are at a particular distance (e.g., from 0.1 cm to 3 cm) of the upper buccal alveolar soft tissue. The alveolar soft tissue can include, for example, the alveolar mucosa. In other embodiments, subpanels 302, 303, and 304 can be configured to be disposed adjacent to the upper lingual alveolar soft tissue. For example, in one or more embodiments, the subpanels 302, 303, and 304 are in contact with the upper lingual alveolar soft tissue; whereas, in other embodiments, the subpanels 302, 303, and 304 are not in contact with the upper lingual alveolar soft tissue but are at a particular distance (e.g., from 0.1 cm to 3 cm) of the upper lingual alveolar soft tissue. Similarly stated, in one or more embodiments, subpanels 302, 303, and/or 304 can be configured to be disposed posterior to the maxillary root area, while in other embodiments, subpanels 302, 303, and/or 304 can be configured to be disposed anterior to the maxillary root area. Similarly, subpanels 306, 307, and/or 308 can be configured to be disposed adjacent to anterior and/or posterior mandibular root area.

In other embodiments, the panels (or other portions of the apparatus) can be disposed at any of the various regions or areas described herein. For example, although in one or more embodiments, the panels are described herein as being in contact with or at a particular distance (e.g., from 0.1 cm to 3 cm) of the upper buccal or lingual alveolar soft tissue, in other embodiments, the panels are configured to be in contact with and/or at a particular distance (e.g., from 0.1 cm to 3 cm) of the lower buccal or lingual alveolar soft tissue. In still other embodiments, an apparatus can include a plurality of panels of which at least a first portion are configured to be in contact with or at a particular distance (e.g., from 0.1 cm to 3 cm) of the upper buccal or lingual alveolar soft tissue, and of which at least a second portion is configured to be in contact with or at a particular distance (e.g., from 0.1 cm to 3 cm) from the lower buccal or lingual alveolar soft tissue when the first portion is in contact with or at the particular distance of the upper alveolar soft tissue.

In one or more embodiments, an apparatus is configured to be disposed only adjacent to the maxillary or mandibular root area. For example, in one or more embodiments, the apparatus is in contact with the maxillary or mandibular root area; whereas, in other embodiments, the apparatus is not in contact with the maxillary or mandibular root area but is at a particular distance (e.g., from 0.1 cm to 3 cm) of the maxillary or mandibular root area. Similarly stated, although FIG. 23 depicts the apparatus 300 including an upper portion (panel 301) and a lower portion (panel 305), in other embodiments, the apparatus has only an upper portion or only a lower portion. Although the apparatus is shown with six panels, in other embodiments, the apparatus can have one or more panels. For example, a single panel can be configured to cover at least a portion of the maxillary and/or mandibular root area or the entirety. In other embodiments, one or more panels can be disposed adjacent to the root area of each tooth. For example, in one or more embodiments, one or more panels are in contact with the root area of each tooth; whereas, in other embodiments, one or more panels are not in contact with the root area of each tooth but are at a particular distance (e.g., from 0.1 cm to 3 cm) of the root area of each tooth.

Although in one or more embodiments the panels described herein cover at least some of the anatomical dimensions (e.g., length) of most tooth roots, variation in soft tissue and boney architecture of individual patients might prevent the panel from covering the apical extent of some tooth roots. In such cases, apical portions of the teeth can receive lower energy density. In one or more embodiments, however, the panels include an embedded LED array that is configured to direct light in the direction of such apical portion(s) or is configured to otherwise increase the intensity in the apical portions of the panels.

In one or more embodiments, such as the embodiment depicted in FIG. 24, an apparatus is configured to wrap over the teeth, such that a first portion of the apparatus is disposed adjacent to the anterior root area and a second portion of the apparatus is disposed adjacent to the posterior root area. In such embodiments, the apparatus is relieved over the anatomical crowns in order to provide freedom of tooth movement when the apparatus is in operation. For example, in one or more embodiments, the first portion of the apparatus is in contact with the anterior root area and/or the second portion of the apparatus is in contact with the posterior root area; whereas, in other embodiments, the first portion of the apparatus is not in contact with the anterior root area and/or the second portion of the apparatus is not in contact with the posterior root area but the first portion and/or the second portion of the apparatus is/are at a particular distance (e.g., from 0.1 cm to 3 cm) of the anterior root area or posterior root area, respectively.

FIG. 25A depicts a cross section of the apparatus of FIG. 23 taken along line X-X. The apparatus 300 can include one or more wires 312, a reflective backing 311, a circuit 313, and one or more emitters 332, encased in the panel 305. In one or more embodiments, the one or more emitters 332 can be partially encased within the panel 305 such that at least a portion of the one or more emitters 332 are exposed and can contact, for example, the alveolar soft tissue of the patient when the apparatus is in the patient's mouth. The panel 305 can be constructed of a substantially transparent, flexible, and/or soft polymer, such as a silicone. In other embodiments, the panel 305 can be a rigid plastic, such as an acrylic. The panel 305 can be shaped to cover a specific region of the patient's mouth when the apparatus is worn by the patient. For example, the panel 305 can have a width and a length effective to cover at least four of the patient's tooth roots. A portion of the panel 309 can have a rounded and/or teardrop shape to provide for patient comfort and to allow the apparatus to adapt to the flange area. The portion of the panel 309 can have any shape that does not include sharp or acute edges as such edges would irritate or be uncomfortable in the depth of the vestibule of the patient's mouth.

In one or more embodiments, the panel 305 is at least partially encased in the apparatus, which can have a shape similar to a mouth guard or to a clear dental aligner and can be constructed of any material suitable for use in the mouth. In this manner, the components encased in the panel 305 are also at least partially encased in the apparatus. In one or more embodiments, the panel 305 is fully encased in the apparatus. The panel 305 and components encased therein can be fluidically sealed within the apparatus so that saliva or another fluid cannot contact the panel 305. Sealing the panel 305 in this manner can provide safety benefits, extend the life of the intra-oral apparatus, and/or require less maintenance. For example, if the panel 305 is not fluidically sealed within the apparatus, then the apparatus might require frequent maintenance to clean fluids and other buildup from the panel 305.

The emitters 332 can be any suitable device that is operable to emit light. The emitters 332 can be, for example, light emitting diodes (LEDs). In one or more embodiments, the emitters 332 are optical fibers (or portions thereof) that emit light. In one or more embodiments, the emitters 332 are devices that are connected to and receive light input from one or more optical fibers. The panel 305 can include any combination of the LED and optical fiber emitters disclosed herein. In one or more embodiments, the emitters 332 can emit monochromatic light having a wavelength of about 620 nm. In other embodiments, the emitters 332 can emit monochromatic light having a wavelength of about 850 nm. In yet other embodiments, the emitters 332 can be configured to emit a light having a wavelength ranging from about 600 nm to about 1200 nm, emit light at more than one wavelength, progress through a range of wavelengths, and/or emit a broad spectrum light or any suitable wavelength or wavelengths. The emitters 332 can be configured to emit light having any wavelength or characteristic described herein. Such wavelengths and characteristics of light are described in more detail herein.

The emitters 332 can be positioned and arranged within the panel 305 in any suitable manner. The emitters 332 can be arranged, for example, so that they cover and irradiate light to a specific region of the mouth when the apparatus is worn by the patient. In one example, each emitter 332 is positioned over and irradiates light to a different tooth root. In another example, the emitters 332 are grouped together into sets so that one set of emitters is positioned over and irradiates light to a first region of the patient's mouth (e.g., a tooth root) while another set of emitters is positioned over and irradiates light to a second, different region of the patient's mouth (e.g., another tooth root). In this manner, the apparatus and the emitters 332 within each corresponding panel 305 can be customized for a specific patient so that particular needs of the orthodontic treatment are met. As noted herein, the panels, and thus the emitters 332, can be in contact with or at a particular distance from the alveolar soft tissue or tooth root. A light dose emitted by the emitters 332 can be more effective for regulating tooth movement the closer the emitters 332 are to the alveolar soft tissue or tooth root, due to a loss of energy that can occur over a distance between the emitters 332 and the tissue or root. In one or more embodiments, however, the power density of light emitted by emitters 332 can be maximized by positioning the emitters 332 in contact with and/or at the particular distance from the tissue or root, as described herein.

In one or more embodiments, as shown in FIG. 25B, a light emitting array 320 has a first portion 321 and a second portion 322. The first portion 321 of the light emitting array 320 is configured to include a greater density of light emitters than a density of light emitters in the second portion 322. In one or more embodiments, the first portion 321 of the light emitting array 320 includes a tighter weave on a portion of a light mat. The first portion 321 of the light emitting array 320 (or light mat) can be disposed at an apical portion of the array. The increased power density at the apex resulting from the tighter weave of the light mat permits a shorter extension to a flange as the increased power at least partially compensates for a shorter extension.

Referring again to FIG. 25A, the circuit 313 can be, for example, a flexible circuit. In one or more embodiments, the circuit 313 includes a controller (not shown) operable to control the operation of one or more emitters 332. In other embodiments, the circuit 313 can be coupled to a controller, such as an intra-oral or extra-oral controller. In one or more embodiments, the controller can independently control each of the one or more emitters 332. For example, using the circuit 313, the controller can collectively and/or individually control the on/off state, the intensity, the frequency, the pulse, the duty factor, and/or any other suitable parameter of the one or more emitters 332. Any one of these parameters can be changed while the apparatus is in use. By powering on one or more of the emitters 332, the controller enables the one or more emitters 332 to emit light and thereby accelerate bone remodeling and/or tooth movement. By powering off one or more of the emitters 332, the controller minimizes the movement of the teeth in the area, as bone remodeling will not have been accelerated by the light. In one or more embodiments, one or more emitters 332 within panel 305 can be on while one or more other emitters 332 within panel 305 are off. In one or more embodiments, when the apparatus is in use, one or more emitters 332 can start in the on state and then, at some later time, switch to the off state. By increasing or decreasing the intensity of the light, the controller can increase or decrease the dosage of light to the patient. A dosage of light is based on intensity and time so, in some instances, increasing the intensity of the light allows a decrease in the amount of time that the light needs to be administered to the patient. As a practical matter, there is a biological threshold of both minimum time and intensity in order to produce a therapeutic result. The controller can operate the emitters 332 at or above this threshold. In one or more embodiments, the intensity of light emitted from one or more emitters 332 within the panel 305 can be increased while the intensity of light emitted from one or more other emitters 332 within the panel 305 is decreased. This increase and decrease can occur, for example, while the apparatus is in use.

The controller can control the frequency and duty factor so that higher peak intensities can be achieved. High peak intensities can be useful in thicker tissues and/or when dosages of light need to be administered at greater depths. In one or more embodiments, a first emitter within the panel 305 can be disposed adjacent to and targeting a bone region that is deeper beneath the alveolar soft tissue than the bone region that a second emitter within the panel 305 is targeting. In these embodiments, the controller can program or control the first emitter so that it emits light having a higher peak intensity than the second emitter. Controlling the duty factor can also protect the emitters from overheating. For example, the controller can operate one or more emitters 332 at a 25% duty factor and at a frequency of 100 Hz such that the emitters 332 are ON for 1/400th of a second and then OFF for 3/400ths of a second. The OFF time would allow the emitters 332 to cool down, thereby avoiding any potential performance degradation associated with higher temperatures.

As disclosed herein, the controller can individually and selectively control the various light emission characteristics of each emitter 332 within the panel 305 and, as a result, each emitter 332 can operate independently of the other emitters 332 within the panel 305. Specifically, each emitter 332 within the panel 305 can emit light having different characteristics, if needed. The panel 305, therefore, can irradiate light at more than one wavelength or otherwise irradiate light having multiple different characteristics. In other embodiments, the controller can collectively control the various light emission characteristics of the emitters 332 within the panel 305. In some instances, all of the emitters 332 within the panel 305 are controlled so that they emit light having the same characteristics. These emitters 332, however, can be operated and controlled independently of emitters within other panels of the apparatus. For example, the emitters 332 can emit light having a wavelength of 850 nm while the emitters within another panel (e.g., subpanel 304 shown in FIG. 23) emit light having a wavelength of 650 nm. Emitted light characteristics, therefore, can vary from panel to panel within the apparatus. In other instances, the emitters 332 within the panel 305 can form groups and the emitters within each group are collectively controlled. For example, the panel 305 can include two groups of emitters 332: the first group of emitters can emit light at a first wavelength and the second group of emitters can emit light at a second, different wavelength. The panel 305, and the emitters 332 therein, can be customized for a specific patient so that an effective amount or dosage of light is administered to the patient and specific regions within the mouth are targeted. This customization can be useful when, for example, one region of the mouth undergoes a different light treatment than another region of the mouth.

In one or more embodiments, the apparatus can include an internal power source, such as a battery (not shown). In other embodiments, the apparatus can include a port, such that the circuit 313 can be coupled to an external power source.

In one or more embodiments, the circuit 313 can include one or more sensors (not shown) to detect the temperature of the apparatus, the patient's alveolar soft tissue and/or the patient's root area. For example, a thermistor or similar temperature measuring device can be placed in the circuitry 313 to monitor the temperature of the emitters 332 (e.g., an LED array) and panel 305 as well as measure the temperature inside the patient's mouth. This information can serve as a method for obtaining temperature-related information as well as monitoring patient compliance. When the circuits are placed in the mouth (i.e., circuit 313 and the circuits from the remaining panels of the apparatus) and when the apparatus emits light, the temperature of the emitters will rise from pre-treatment ambient temperature closer to normal body temperature. By monitoring the change in temperature, the controller can monitor the period of time that the emitters 332 are in the mouth, based on the period of time the temperature is elevated and close to body temperature. Alternatively, as described in more detail with reference to FIGS. 40A-40C, a photodetector can be placed in the circuit 313 and/or with the emitters 332 to measure a property. e.g., wavelength or intensity, of light reflected from the alveolar soft tissue. This configuration can serve as a method for monitoring patient compliance and also serve as a failsafe mechanism to ensure that the emitters 332 do not operate unless the apparatus is within the mouth of the patient.

The reflective backing 311 can be a metallic foil or other suitable reflective material operable to cause the light emitted by the emitters 311 to be directed in a desired direction, in one or more embodiments, substantially one direction, e.g., no more than about 1 to about 10 degrees of a specific direction. For example, the reflective backing 311 can define the back of the apparatus, such that the light is directed towards the alveolar soft tissue or root area of the patient (e.g., the region beneath the alveolar soft tissue that includes bone and roots).

The wires 312 can be super-elastic wires operable to cause the apparatus to conform to the alveolar soft tissue and/or gingiva. In one or more embodiments, the wires 312 can produce a relatively large orthodontic and/or orthopedic force, such as a force operable to urge one or more teeth to move. The force can be, for example, from about 10 to about 1000 grams of force. In one or more embodiments, the force is a heavy force. In other embodiments, the apparatus can be a portion of and/or be coupled to a separate intra-oral apparatus, such as orthodontic braces, retainers and/or any other suitable functional appliance. In some such embodiments, the separate intra-oral apparatus can produce a force in combination with or in lieu of the wires 312 producing a force.

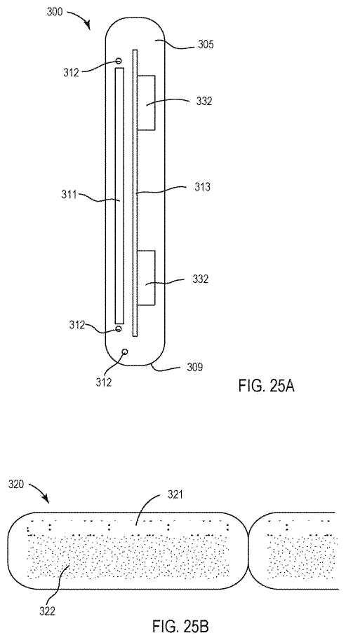

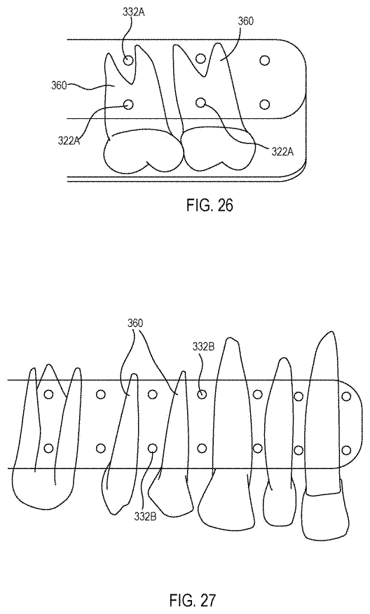

FIGS. 26-28 are schematic diagrams of an apparatus of the invention. As shown in FIG. 26, in one or more embodiments, one or more emitters 332A can be disposed over the roots of one or more teeth 360. In other embodiments, as shown in FIG. 27, one or more emitters 332B can be disposed between the roots of one or more teeth 360. In such embodiments, a mask can be applied to the apparatus and/or the root area of each tooth 360 to prevent the root area of the teeth 360 from being exposed to the light. As is disclosed herein, the mask blocks the light irradiated from the one or more emitters 332A and/or 332B so that little or none of the light reaches the area covered by the mask. The mask can be a tooth mask. The mask can be opaque and/or reflective. In one or more embodiments, the mask includes an adhesive surface so that the mask can be placed and adhered to an outer surface of the apparatus at a location where light is desired to be blocked. In one or more embodiments, the mask is in the form of a sticker. The adhesive surface of the mask can contact and/or cover one or more panels (or a portion thereof). In one or more embodiments, the opposing outer surface of the mask (or a portion thereof) can contact the alveolar soft tissue (e.g., the alveolar mucosa) when the mask is adhered to the apparatus and the apparatus is in the patient's mouth. In one or more embodiments, more than one mask can be applied to the apparatus and/or the root area of each tooth 360 to prevent the root area of the teeth 360 from being exposed to the light. In some such embodiments, more than one type of mask can be applied. For example, both an opaque mask and a reflective mask can be applied to the apparatus.

In other embodiments, as shown in FIG. 28, one or more emitters 332 can be operable to irradiate the maxillary suture, for example, the midline of the maxillary suture. In one or more embodiments, the one or more emitters 332C emit light directed towards the maxillary suture before, during, and/or after an orthopedic force is exerted on the maxillary suture. The orthopedic force can be exerted by an orthodontic appliance, such as, for example, a Rapid Maxillary Expansion (RME) appliance. A RME appliance can exert orthopedic forces on the patient's molars to open up and expand the maxillary suture for skeletal expansion of the upper jaw (as opposed to an orthodontic expansion where only the teeth move). Light therapy can be useful in these embodiments to accelerate the rate at which the maxillary bone grows and the gaps caused by the skeletal expansion are filled. In one or more embodiments, the present methods are useful for accelerating the fill of bone and/or decreasing the potential for relapse or narrowing of the maxillary arch after orthodontic appliance removal. In one or more embodiments, the one or more emitters 332C emit light directed towards the midline of the palate such that boney regeneration is simulated through light therapy. In one or more embodiments, the apparatus shown in FIG. 28 is customized to fit around the RME appliance or other like fixed orthodontic expander. In one or more embodiments, the apparatus shown in FIG. 28 includes the one or more emitter 332C that irradiate the maxillary suture as well as one or more other emitters that irradiate the alveolar soft tissue.

The emitters 332A, 332B and/or 332C can operate in a manner similar to the emitters 332 depicted in FIG. 25A.

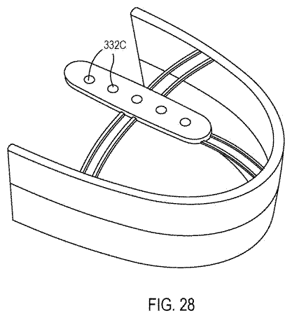

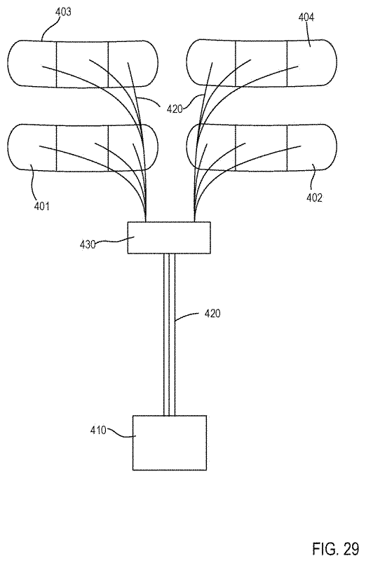

FIG. 29 is a schematic diagram of an apparatus, according to an embodiment. The apparatus includes four panels 401, 402, 403, 404, a light source 410, one or more optical fiber cables 420, and a controller 430. The panels 401, 402, 403, and/or 404 can be configured to be disposed adjacent to the root area of the upper and/or lower jaw. For example, in one or more embodiments, the panels 401, 402, 403, and/or 404 are in contact with the root area of the upper and/or lower jaw; whereas, in other embodiments, the panels 401, 402, 403, and/or 404 are not in contact with the root area of the upper and/or lower jaw but are at a particular distance (e.g., from 0.1 cm to 3 cm) of the root area of the upper and/or lower jaw. Such configurations can eliminate the need to place electronics in the oral cavity.

The light source 410 can be operable to emit light. For example, in one or more embodiments, the light source 410 can output monochromatic light. For example, the light source 410 can be a laser, an LED, and/or any other suitable light source. The light source 410 can be configured to emit a light having a wavelength ranging from about 600 nm to about 1200 nm, emit light output at more than one wavelength, progress through a range of wavelengths, and/or emit a broad spectrum light output or any suitable wavelength or wavelengths. The light source 410 can output light with any wavelength or characteristic described herein.

The light can be conveyed from the light source 410 to the controller 430 via one or more optical fibers 420. The controller 430 can be, for example, an optical switch. The controller 430 can be operable to selectively transmit light from the light source 410 to the panels 401, 402, 403, and/or 404 via one or more optical fibers 420. For example, the controller 430 can collectively and/or individually control the on/off state, the intensity, the frequency, the pulse, the duty factor, and/or any other suitable parameters of the light that is delivered to the panels 401, 402, 403, and/or 404. The controller 430 can operate similar to the controller depicted in FIG. 25A.

In one or more embodiments, more than one optical fiber 420 can be directed to each panel. The optical fiber can terminate adjacent to (e.g., from 0.1 cm to 3 cm) or at the root area, similar to the apparatuses shown and described with reference to FIGS. 26 and 27. Thus, each optical fiber can direct light from the light source 410 to the root area. By providing more than one fiber 420, light from the source 410 can be directed and/or controlled to irradiate a specific portion of the root area. In this way, the controller 430 can selectively apply light to the root area of one or more teeth, similar to the emitters 332, 332A, and/or 332B as shown and described herein with reference to FIGS. 25, 26 and 27.

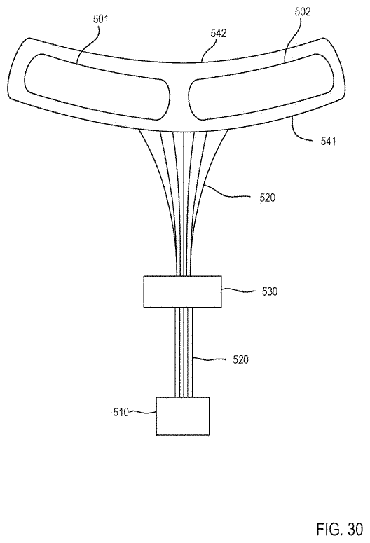

FIG. 30 is a schematic diagram of an apparatus, according to an embodiment. The apparatus includes two panels 501, 502, a light source 510, an optical fiber ribbon 520, and a controller 530. The panels 501 and 502 can be configured to be disposed adjacent to the root area of the upper jaw as well as the root area of the lower jaw. As is disclosed herein, the panels 501 and 502, for example, can first be disposed adjacent to the root area of the upper jaw: then, after orthodontic treatment of the upper jaw is complete, the panels 501 and 502 can be removed from the upper jaw and placed on the lower jaw such that they are disposed adjacent to the root area of the lower jaw. In one or more embodiments, the panels 501, 502 are in contact with the root area of the upper and/or lower jaw; whereas, in other embodiments, the panels 501, 502 are not in contact with the root area of the upper and/or lower jaw but are at a particular distance (e.g., from 0.1 cm to 3 cm) of the root area of the upper and/or lower jaw. Such configurations can eliminate the need to place electronics in the oral cavity.

The panels 501, 502 can define an upper portion 542. The cross-section of the upper portion 542 can be rounded and/or teardrop shaped (similar to the portion of the panel 309 depicted in FIG. 25A) to provide for patient comfort and to allow the apparatus to adapt to the flange area of the upper and lower jaw. For example, as is disclosed herein, the apparatus can be worn on the upper jaw so that the upper portion 542 of the panels 501, 502 adapt to the upper flange area; then, the apparatus can be removed from the upper jaw, flipped upside down, and then installed on the lower jaw such that the upper portion 542 is now disposed in the lower flange area. In this manner, the upper portion 542 of the panels 501, 502 is configured to fit and adapt to both the upper and lower flange area of the patient's mouth. In one or more embodiments, the panels 501, 502 can also define a lower portion 541 that has a rounded and/or teardrop shaped cross-section so that, for example, the apparatus can be removed from the upper jaw and installed on the lower jaw without having to flip the apparatus. Here, the lower portion 541, as opposed to the upper portion 542, is disposed and configured to fit in the lower flange area.

The portions 541, 542 of the panels 501, 502 can have any shape that does not include sharp or acute edges as such edges would irritate or be uncomfortable in the depth of the vestibule of the patient's mouth. In one or more embodiments, the portions 541 and/or 542 have a shape or cross-sectional shape that disperses forces and minimizes pressure points which would cause discomfort for the patient. In one or more embodiments, the portions 541 and/or 542 have a thicker cross-section than the remaining portions of the panels 501, 502. In this manner, the portions 541 and/or 542 can deflect the delicate mucosal soft tissue and allow full extension of the flanges with little or no discomfort to the patient. More specifically, the portions 541 and/or 542 can deflect buccal tissue away from the alveolar mucosa.

The light source 510 can be operable to emit light in the same manner as the light source 410 in reference to FIG. 29. The light can be conveyed from the light source 510 to the controller 530 via the optical fiber ribbon 520. The controller 530 can be operable to selectively transmit light from the light source 510 to the panels 501 and/or 502 via the optical fiber ribbon 520 in the same manner as the controller 430 in reference to FIG. 29. For example, the controller 530 can collectively and/or individually control the on/off state, the intensity, the frequency, the pulse, the duty factor, and/or any other suitable parameters of the light that is delivered to the panels 501 and/or 502.

The optical fiber ribbon 520 can be coupled to the apparatus, as shown in FIG. 30, such that one or more optical fibers of the ribbon 520 are electrically connected and/or directed to each panel 501, 502. For example, one or more of the optical fibers in the ribbon 520 can terminate adjacent to (e.g., from about 0.1 cm to 3 cm) or at the root area, similar to the apparatuses shown and described with reference to FIGS. 26 and 27. Thus, each optical fiber of the ribbon 520 can direct light from the light source 510 to the root area in the same manner as the optical fibers 420 in reference to FIG. 29. The optical fibers of the ribbon 520 can be configured to optically couple the panels 501, 502 together. The optical fiber ribbon 520 can have one or more optical fibers for example, in one or more bundles, therein. For example, the ribbon 520 can have anywhere from 1 fiber to 500 fibers for each panel 501, 502 depending on the specific light emission technology or pattern used for the treatment. The optical fiber ribbon 520 can have any suitable shape and/or size such that the ribbon can comfortably extend from the apparatus to outside the patient's mouth. The ribbon 520 can, for example, have a width of about 0.5 cm to about 1.0 cm. Although the apparatus of FIG. 30 is illustrated and described as having a single ribbon that electrically couples to both panels 501 and 502, in other embodiments, the apparatus includes more than one ribbon. For example, in one or more embodiments, the apparatus includes two ribbons. In one or more embodiments, one ribbon can be electrically connected to the panel 501 and the other ribbon can be separately electrically connected to the panel 502. In one or more embodiments, the ribbon 520 is a woven fiber-optic fabric. More specifically, the ribbon 520 in this embodiment can be comprised of one or more optical fibers that are woven into a fabric. In one or more embodiments, light from the woven fiber optic fabric is emitted at about 90 degrees or is emitted perpendicular to the plane of the fabric. An example of a woven fiber optic fabric that is useful with the apparatus of FIG. 30 is LightMat®, which is commercially available from Lumitex, Inc. (http://www.lumitex.com/).

As disclosed herein, the apparatus in FIG. 30 can be installed on either the upper or lower jaw. In one or more embodiments, the apparatus in FIG. 30 is installed during orthodontic treatment. For example, at the outset of the treatment, the apparatus can be installed on the upper jaw such that the upper portion 542 of the apparatus is disposed in the upper flange area. Then, at a later time during the treatment, the patient can remove the apparatus from the upper jaw and install the apparatus on the lower jaw for the remainder of the treatment. In one or more embodiments, the apparatus can be installed on the lower jaw such that the lower portion 541 of the apparatus is disposed within the lower flange area. In this embodiment, the apparatus remains right-side up. In another embodiment, however, the apparatus can be installed on the lower jaw such that the upper portion 542 of the apparatus is disposed within the lower flange area. In other words, after the patient removes the apparatus from his or her upper jaw, he or she rotates the apparatus 180 degrees so that it is upside down and then installs the apparatus on the lower jaw. The upper portion 542, in this embodiment, fits both the upper and lower flange area.

In one or more embodiments, the apparatus includes an electronic device, such as a position sensor, that can determine the position or orientation of the apparatus relative to the patient's mouth. More specifically, in embodiments where the apparatus is turned upside down (e.g., rotated 180 degrees) for installment on the lower jaw, the apparatus can include an electronic device that determines the apparatus's position or orientation in a patient's oral cavity during an orthodontic treatment. For example, the sensor can determine whether the apparatus is being worn on the upper jaw or on the lower jaw. Such an electronic device can be useful in monitoring compliance during orthodontic treatment. In one or more embodiments, the electronic device can be one or more switches, sensors, and/or the like.

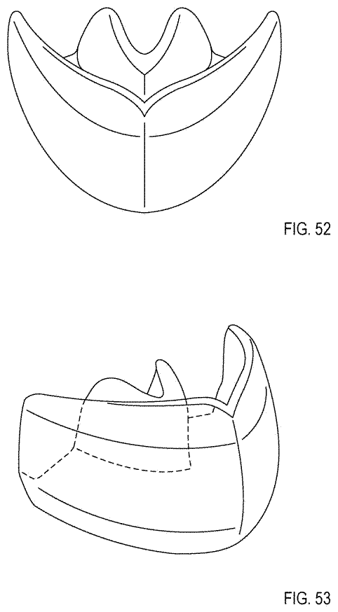

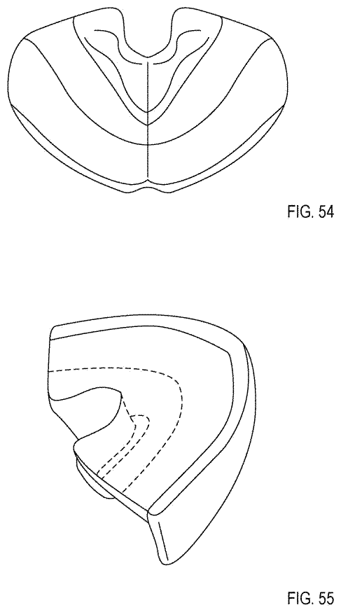

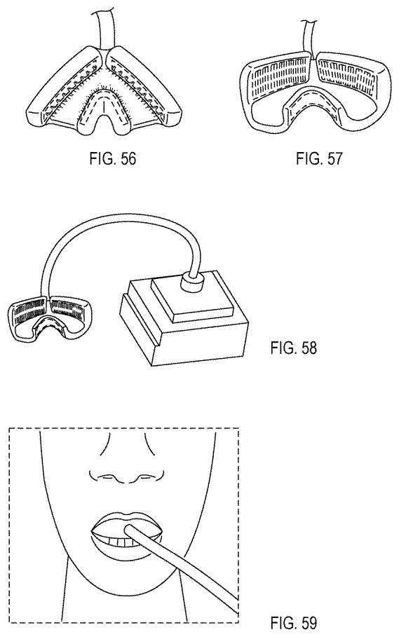

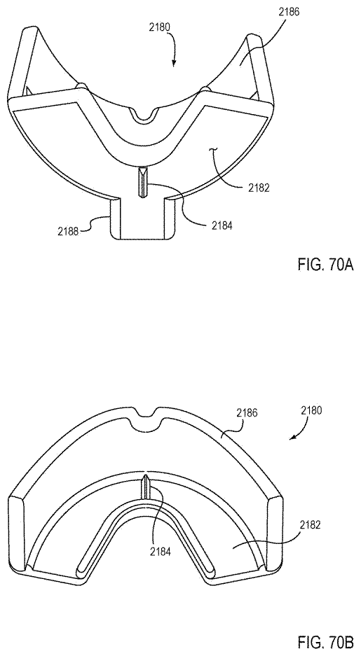

Any of the apparatus illustrated and described herein with reference to FIGS. 23-30 can have any number of panels, which can operate and function in any manner described herein. Although not necessarily illustrated, in one or more embodiments, the panels can be coupled together and/or encapsulated within one or more units. For example, the panels 403 and 404 in FIG. 29 can be coupled together and/or encapsulated in a single unit, similar to a mouth guard that fits the upper teeth. The panels 401 and 402 can likewise be coupled together and/or encapsulated within a single unit, similar to a mouth guard that fits the lower teeth. Example of mouth guards including light emitting panels are shown in FIGS. 52-59 and FIGS. 65-72. It should be noted that although the mouth guard is shown in FIG. 59 positioned with respect to the upper teeth of the patient, the mouth guard is also configured to be positioned with respect to the lower teeth of the patient. In another example, the panels 403 and 404 can be coupled together such at least a portion of the panel 403 overlaps a portion of the panel 404. The panel 403 in this example can emit light at the same wavelength as or at a wavelength different from the panel 404. Furthermore, power output and light treatment intensity can be increased by the layering or overlapping of one or more panels.

In one or more embodiments, any of the intra-oral apparatuses described herein can include a handheld controller that houses one or more of a microprocessor, menu-driven software and an LCD screen. The controller can be programmed to calculate and/or monitor one or more light therapy sessions and their duration. A user interface can display session information to the patient so that, for example, the patient is aware of the number of sessions completed and the time remaining in each session. The controller can use any suitable power supply including, for example, a UL-certified power supply. In one or more embodiments, the intra-oral apparatus can include four treatment arrays, each of which can include a flexible printed circuit board and a set of LEDs mounted to a contoured heat sink and infrared-transmissible lens, in one or more embodiments, a plastic lens, and having conductive cables that attach to the controller.

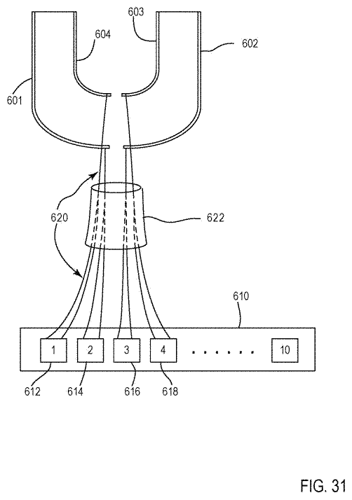

FIG. 31 is a schematic diagram of an apparatus, according to an embodiment of the invention. The apparatus can be configured for intra-oral light therapy of a patient. The apparatus includes four panels 601, 602, 603, and 604, a light source 610, and optical fibers 620. The panels 601, 602, 603 and 604 can be configured to be disposed adjacent to the root area of the upper jaw as well as the root area of the lower jaw, for example, in a similar manner as described herein in reference to FIGS. 23-30. More specifically, panels 601 and 602 can be disposed adjacent to the anterior root area of the upper jaw (or lower jaw), and panels 603 and 604 can be disposed adjacent to the posterior root area of the upper jaw (or lower jaw). In other words, the panels 601, 602, 603, 604 can be configured to be disposed anterior (for panels 601, 602) and posterior (for panels 603, 604) to each of the maxillary root area and the mandibular root area. In this manner, in use, the panels 601, 602 can be configured to emit light in a direction towards the panels 603,604, and panels 603, 604 can be configured to emit light in a direction towards the panels 601, 602. The panels 601, 602, 603 and 604, for example, can first be disposed adjacent to the respective anterior or posterior root area of the upper jaw; then, after orthodontic treatment of the upper jaw is complete, the panels 601, 602, 603 and 604 can be removed from the upper jaw and placed on the lower jaw such that they are disposed adjacent to the respective anterior or posterior root area of the lower jaw. In one or more embodiments, the panels 601, 602, 603 and 604 are in contact with the root area of the upper and/or lower jaw; whereas, in other embodiments, the panels 601, 602, 603 and 604 are not in contact with the root area of the upper and/or lower jaw but are at a particular distance (e.g., from 0.1 cm to 3 cm) of the root area of the upper and/or lower jaw. In one or more embodiments, the panels 601, 602 can be configured to be disposed adjacent to (e.g., in contact with or at a particular distance from) the upper and/or lower buccal lingual alveolar soft tissue and panels 603, 604 can be configured to be disposed adjacent to the upper and/or lower lingual alveolar soft tissue. Such configurations can eliminate the need to place electronics in the oral cavity.

The panels can be similar in one or more respects or identical to any panel described herein, including, for example, those described in reference to FIGS. 22-30. Each panel 601, 602, 603, 604 is associated with a bundle of optical fibers 620 that extend to the light source 610. More specifically, each panel 601, 602, 603, 604 is associated with an emitter 632 of the light source 610 via a bundle of optical fibers 620. In this manner, each panel 601, 602, 603, 604, and any housing (not shown in FIG. 31) to which the respective panel is coupled, is optically coupled to the emitter 632 of the light source 610.

The light source 610 can be operable to emit light in the same manner as the light source 410 in reference to FIG. 29 and/or light source 510 in reference to FIG. 30. The light source 610 can include, for example, one, two three, four or more (e.g., ten) LEDs (including, for example, the LEDs 612, 614, 616, 618 shown in FIG. 31). At least a portion of the light source 610, for example, including the LEDs 612, 614, 616, 618, can be disposed in an external housing of the apparatus, at least a portion of which is configured to be disposed extra-orally when the panels 601, 602, 603, 604 are disposed in the oral cavity adjacent the root area as described herein. For example, the external housing of the apparatus can be extended through an opening formed by the patient's lips when the panels 601, 602, 603, 604 are disposed in the oral cavity adjacent the root area as described herein.

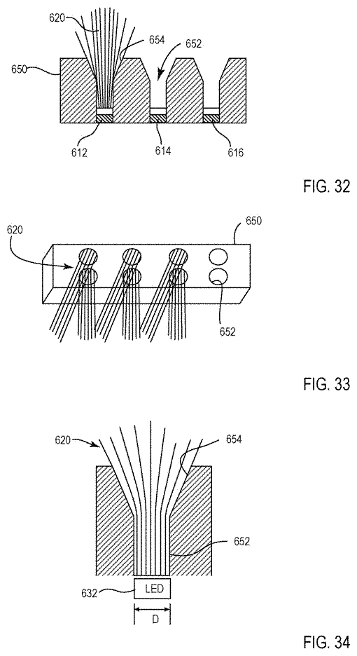

The apparatus can include a manifold 650 defining one or more openings 652 therethrough. Each opening 652 of the manifold 650 can include a tapered surface portion 654 such that at least a portion of the opening 652 is funnel-shaped. A bundle of optical fibers 620 extending between the light source 610 (e.g., one of the LEDs 612, 614, 616, 618) and one of the panels (e.g., panel 601) can be disposed through the opening 652 of the manifold 650. As illustrated in FIG. 32, ends of the optical fibers 620 in the bundle are seated over, or adjacent, the LED 612. For example, in one or more embodiments, ends of the optical fibers 620 in the bundle are seated over an LED package (e.g., LED package 632, shown in FIG. 34, which can include, for example, LED 612) or other suitable light source. At least one of the portion of the opening 652 of the manifold 650 proximate to the LED package (or other emitter) 632, or the LED package (or other emitter) 632, can have a diameter equal to or narrower than a diameter of the optical fiber bundle. For example, as shown in FIGS. 33 and 34, each of the optical fiber bundle 620 and the manifold 650 opening 652 proximate to the LED package 632 has a diameter D. The use of the funnel-shaped manifold allows for organization of the optical fibers 620 into groups of smaller bundles, thereby eliminating any need for bulky on-board ferrules. Such organization of the optical fibers 620 via the manifold 650 also provides for addressing of an individual panel 601, 602, 603, 604, as described in more detail herein.

The light can be conveyed from the LEDs (e.g., LED 612, 614, 616, 618) of the light source 610 to the panels 601, 602, 603 and/or 604 via the optical fibers 620. For example, a controller (not shown in FIG. 31) can collectively and/or individually control the on/off state, the intensity, the frequency, the pulse, the duty factor, and/or any other suitable parameters of the light that is delivered to the panels 601, 602, 603 and/or 604.

The optical fibers 620 can be coupled to the apparatus, as shown in FIG. 31, such that bundles of optical fibers 620 are electrically connected and/or directed to each panel 601, 602, 603, 604. For example, the optical fibers 620 can be coupled such that proximal ends of the optical fibers 620 are coupled, or are otherwise adjacent, at least one of the light source 610 and the manifold 650 and such that distal ends of the optical fibers 620 are coupled, or are otherwise adjacent, to one or more panels 601, 602, 603 and/or 604. One or more of the optical fibers 620 (i.e., the distal end of one or more of the optical fibers) can terminate adjacent to (e.g., from about 0.1 cm to 3 cm) or at the root area, similar to the apparatuses shown and described with reference to FIGS. 26 and 27. Thus, each optical fiber 620 can direct light from the light source 610 to the root area in the same manner as the optical fibers 420 in reference to FIG. 29. The optical fibers 620 can be configured to optically couple the panels 601, 602, 603 and/or 604 together. The optical fibers 620 can be bundled in any suitable number of fibers. For example, each panel 601, 602, 603 and/or 604 can be associated with a bundle of anywhere from 1 fiber to 500 fibers depending on the specific light emission technology or pattern used for the treatment. The optical fibers 620 can have any suitable shape and/or size such that the fibers can comfortably extend from the panel to the light source 610 disposed outside the patient's mouth. The optical fibers 620 can, for example, have a collective width of about 0.5 cm to about 1.0 cm. A collar 622 can be disposed about one or more of the bundles of optical fibers 620 to maintain fiber bundles together. The apparatus shown in FIGS. 31-34 can be installed on either the upper or lower jaw, for example, during orthodontic treatment.

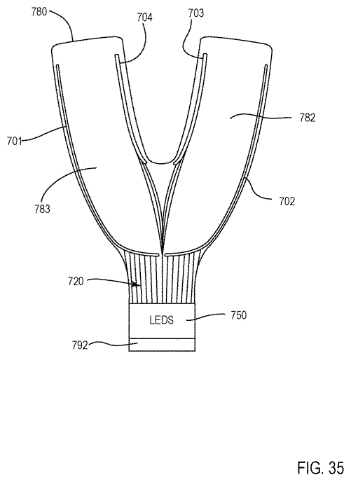

FIG. 35 is a schematic diagram of an apparatus, according to an embodiment of the invention. The apparatus can be configured for intra-oral light therapy of a patient. The apparatus can be similar in one or more respects or identical to other intra-oral light-therapy apparatuses described herein, including, for example, the apparatus described herein with reference to FIGS. 31-34. The apparatus includes an intra-oral housing 780 and an external housing 790 that extends from a front portion of the intra-oral housing (e.g., an anterior portion thereof, for example a midpoint or center point of a front/anterior surface of the intra-oral housing) such that at least a portion of the housing is disposed extra-orally when the intra-oral housing is disposed within the oral cavity.

The intra-oral housing 780 includes one or more panels 701, 702, 703, 704. The panels 701, 702, 703, 704 can include light emitting arrays, fiber mats, organic LEDs (“OLEDs”), or any suitable combination of the foregoing. The panels 701, 702, 703, 704 can be configured to be disposed within the patient's oral cavity in any manner described herein with reference to panels 601, 602, 603, 604.

The intra-oral housing 780 can be connected between the panels 701, 702 configured to be positioned adjacent the anterior root area of the jaw (or the buccal alveolar soft tissue) and the panels 703, 704 configured to be positioned adjacent the posterior root area of the jaw (or the lingual alveolar soft tissue). In one or more embodiments, the intra-oral housing 780 includes a lower portion configured to extend between a lower portion (not shown in FIG. 35) of the panel 701 and a lower portion (not shown in FIG. 35) of the panel 704, and similarly between a lower portion (not shown in FIG. 35) of the panel 702 and a lower portion (not shown in FIG. 35) of the panel 703. In this manner, the intra-oral housing 780 can include recessed portions 782, 783 defined by the lower portion of the intra-oral housing and an upper portion of the intra-oral housing including the panels 701, 702, 703, 704. The recessed portions 782, 783 can be configured to receive, or be disposed about, at least a portion of the patient's dentition. More specifically, the recessed portions 782, 783 are configured to have a depth sufficient to receive at least a portion of the patient's dentition in the lower portion of the intra-oral housing such that the upper portion of the intra-oral housing including the panels 701, 702, 703, 704 is disposed adjacent and/or in contact with the alveolar soft tissue or the root area of the upper and/or lower jaw.

Optical fibers 720 extend between the panels 701, 702, 703, 704 and a light source (not shown in FIG. 35) disposed in the external housing 790 (e.g., at a front portion of the external housing configured to remain outside the oral cavity when the apparatus is in use) such that one or more optical fibers are electrically connected and/or directed to each panel 701, 702, 703, 704. For example, one or more of the optical fibers 720 can terminate adjacent to (e.g., from 0.1 cm to 3 cm) or at the root area, similar to the apparatuses shown and described with reference to FIGS. 26 and 29. Thus, each optical fiber 720 can direct light from the light source to the root area in the same manner as the optical fibers 420 described in reference to FIG. 31. The optical fibers 720 can be configured to optically couple any combination of the panels 701, 702, 703, 704 together. The apparatus can have any suitable number of optical fibers. For example, the apparatus can have anywhere from 1 fiber to 500 fibers for each panel 701, 702, 703, 704 depending on the specific light emission technology or pattern used for the treatment. The optical fibers 720 can be connected to the light source by a manifold 750. The manifold can be similar in one or more respects or identical to the manifold 650 described with respect to FIGS. 32-34, and thus is not described in detail herein.

The light source can be similar in one or more respects or identical to the light source 610 described herein with reference to FIGS. 31-34, and thus is not described in detail herein. The light source can be operable to emit light. For example, in one or more embodiments, the light source can output monochromatic light. For example, the light source can be or include one or more of a laser, an LED, and/or any other suitable light source. The light source can be configured to emit a light having a wavelength ranging from about 600 nm to about 1200 nm, or at any wavelength or wavelength range disclosed herein; emit light output at more than one wavelength; progress through a range of wavelengths; and/or emit a broad spectrum light output or any suitable wavelength or wavelengths. The light source can output light with any wavelength or characteristic described herein.

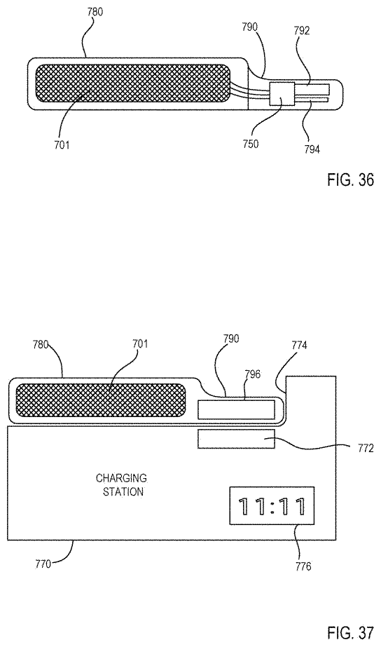

The external housing 790 includes a power source 792 and an electronic circuit 794, as shown in FIG. 36. The power source 792 can be a battery, including, for example, a rechargeable battery. The electronic circuit 794 can include a circuit board. The electronic circuit 794 and any associated electronics can be configured to control the apparatus, i.e., during an orthodontic treatment. For example, the electronic circuit is configured to control at least one of an operational state of the light source and/or optical fibers 720, a wavelength, an intensity, a frequency, or a duration of light emission. Because the apparatus does not require any physical connection to external components during the treatment (e.g., does not require connection to an external light source, external controller, or external power source), the apparatus can be characterized as being self-contained.

The apparatus can be configured to determine whether the apparatus is in an upright or upside down (e.g., rotated 180 degrees) position or orientation (i.e., whether the apparatus is oriented with respect to the upper jaw or the lower jaw). For example, in one or more embodiments, the external housing 790 includes at least one of a position sensor, a gyroscope and an accelerometer. The gyroscope and/or the accelerometer can be include one or more sensors configured to determine the position (or orientation) of the apparatus.

In one or more embodiments, the apparatus is a component of a system that also comprises a charging station 770, as shown in FIG. 37. The charging station 770 defines a receiving portion 774 configured to receive at least a portion of the apparatus (e.g., at least a portion of the intra-oral housing 780 and/or the external housing 790), a connection assembly 772, and a display 776. The connection assembly 772 is configured to facilitate charging (or recharging) of the power source 792 disposed in the external housing 790. In one or more embodiments, the connection assembly 772 provides for a physical or wired connection for coupling to a connection assembly 796 of the apparatus to facilitate charging of the power source 792. For example, the connection assembly 772 can include a socket disposed on one of the apparatus or the charging station 770 and a corresponding plug disposed on the other of the apparatus or the charging station. In one or more embodiments, the connection assembly 772 is configured for wirelessly charging the power source 792. For example, the connection assembly 772 can be configured to inductively charge the power source 792. The display 776 of the charging station 770 is configured to display information associated with the apparatus and/or the charging station. For example, the display 776 can be configured to display information related to a status or amount of the charge of the power source 792, parameters associated with a treatment protocol, and/or instructions for using one of the charging station 770 or the apparatus. In one or more embodiments, the charging station 770 is configured for uni-directional or bi-directional communication with the apparatus. In this manner, information associated with the treatment protocol and/or treatment history (e.g., patient usage or compliance with the prescribed treatment protocol), including updates including any changes to the treatment protocol and/or treatment history since the most recent information transfer between the apparatus and the charging station.

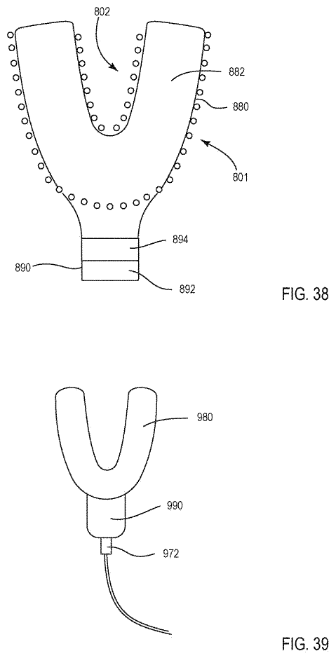

FIG. 38 is a schematic illustration of an apparatus, according to an embodiment. The apparatus can be configured for intra-oral light therapy of a patient. The apparatus can be similar in one or more respects, and include components similar in one or more respects, or identical, to the apparatus and associated components described herein, including those described herein with reference to FIGS. 35-37. For example, the apparatus includes an intra-oral housing 880 and an external housing 890. The intra-oral housing 880 is configured to be disposed in an oral cavity. The external housing 890 is extended from a front of the intra-oral housing 880 such that at least a portion of the external housing can extend through an opening formed by the patient's lips and such that at least a portion of the external housing is outside of the oral cavity when the intra-oral housing is disposed in the oral cavity.

The intra-oral housing 880 can be configured to be positioned within the oral cavity in any suitable manner described herein. The intra-oral housing 880 includes a first light emitting array 801 configured to be disposed adjacent the anterior root area of an upper and/or lower jaw (and/or the buccal alveolar soft tissue) and a second light emitting array 802 configured to be disposed adjacent the lingual root area of an upper and/or lower jaw (and/or the lingual alveolar soft tissue). For example, from a top view, as schematically shown in FIG. 38, the intra-oral housing 880 can have a shape similar to the shape of a U or a horseshoe. Thus, stated another way, the first light emitting array 801 is disposed on an outer portion of the U-shape of the intra-oral housing 880, and the second light emitting array 802 is disposed on an inner portion of the U-shape of the intra-oral housing. In this manner, in use, the light emitting arrays 801, 802 can be configured to emit light in a direction towards the light emitting arrays 803,804, and light emitting arrays 803, 804 can be configured to emit light in a direction towards the light emitting arrays 801, 802.

The light emitting arrays 801, 802 are at least partially embedded in a material of which the intra-oral housing 880 is constructed. The intra-oral housing 880 can be constructed of any suitable material, including, for example, silicone or another soft, e.g., malleable, material. For example, the light emitting arrays 801, 802 can include LEDs. OLEDs, light emitting semiconductors, or any suitable combination thereof, at least partially embedded in the material of which the intra-oral housing 880 is constructed. In one or more embodiments, the light emitting arrays 801, 802 are fully embedded in the intra-oral housing 880 material.

The intra-oral housing 880 can define a recessed portion 882 in a similar manner as that described with respect to recessed portion 782 in reference to FIG. 35, and thus the recessed portion 782 is not described in detail herein.

The external housing 890 includes a power source 892, electronic circuit 894, and an orientation-sensing mechanism (not shown in FIG. 38). In this manner, the apparatus can be characterized as being self-contained. The power source 892 (e.g., a battery) is configured to provide power to the light emitting arrays 801, 802 via the electronic circuit 894. The electronic circuit 894 can be configured to control the apparatus, e.g., during an orthodontic treatment. The orientation-sensing mechanism is configured to determine a position or orientation of the apparatus, e.g., whether the apparatus is upright for positioning with respect to the upper jaw or upside down for positioning with respect to the lower jaw. The orientation-sensing mechanism can include at least one of a position sensor, a gyroscope (e.g., a semi-gyroscope) and an accelerometer.

The apparatus can be configured for use with a charging station, such as charging station 770, in a similar manner as the apparatus described herein with reference to FIGS. 35-37.

Although the apparatuses described with respect to FIGS. 35-38 have been described as being configured for use with a charging station (e.g., charging station 770), in other embodiments, a self-contained apparatus can be differently configured for charging the power source and/or controlling the emission of light by the apparatus. For example, referring to FIG. 39, an apparatus includes an intra-oral housing 980 configured to be disposed in an oral cavity and an external housing 990 configured to extend through an opening formed by the patient's lips such that at least a portion of the external housing is outside of the oral cavity when the intra-oral housing is disposed in the oral cavity. The intra-oral housing 980 can be similar in one or more respects, and can include components that are similar or identical to those of any intra-oral housing or apparatus for intra-oral light therapy described herein. The external housing 990 can be similar in one or more respects, and can include components that are similar or identical to those of external housings 790 and 890 described herein.

A power source (not shown in FIG. 39) in the external housing 990 is configured to be charged via a connector 972 (e.g., a USB mini- or microplug). The apparatus can be electronically linked, or paired, with an external electronic device, such as a mobile phone, including smartphones (e.g., an iPhone®, or an Android™ based device). The apparatus can be configured for at least one of wireless uni-directional or wireless bi-directional communication with the external electronic device, such as via a Bluetooth® or other wireless connection. For example, the apparatus can be configured to transmit to the external electronic device information associated with patient usage and/or treatment protocol compliance, and can be configured to receive from the external electronic device information associated with a medical, e.g., an orthodontic, treatment. An application loaded onto the external electronic device can be useful for monitoring and controlling the orthodontic treatment using the apparatus and/or to record and review patient usage history and/or prescribed treatment protocol compliance history.

In one or more embodiments, an apparatus according to an embodiment is configured to detect an amount of light, e.g., its intensity or duration that is irradiated at, absorbed by or reflected by a patient's periodontia (e.g., a portion of the root area of the upper and/or lower jaw and/or the alveolar soft tissue) by the apparatus. In this manner, for example, an apparatus according to an embodiment can be configured to assess patient compliance with a prescribed orthodontic treatment protocol, as described herein. Referring to FIG. 40A, an apparatus according to an embodiment can include a light emitting array 1101, which can be similar or identical to any light emitting array described herein, and one or more photodetectors 1102. In one or more embodiments, the light emitting array 1101 includes one or more emitters operable to irradiate a region of or associated with a portion of the root area of the upper and/or lower jaw and/or the alveolar soft tissue. At least a portion of the photodetectors 1102, such as one or more sensors of the photodetectors, are configured to be positioned within the oral cavity to detect transmission or reflection of light (i.e., photons) emitted by the light emitting array 1101 from the alveolar soft tissue (and associated alveolar mucosa). For example, the photodetectors 1102, or a sensor thereof, can be positioned on a palatial surface within the oral cavity, and can be in electrical communication with a portion of the photodetector disposed outside of the oral cavity. Detection of the light transmission by the photodetectors 1102 during an orthodontic treatment activates the photodetectors every few seconds. The apparatus can be configured to power off if no reflected light is detected by the photodetectors for a predetermined period of time. The apparatus can be configured to store a record of the history of light detection and of the apparatus being powered off because of a lack of detection. Such usage information can be useful to determine whether the patient is compliant with a prescribed orthodontic treatment protocol. This configuration can also serve as a failsafe mechanism to ensure that the light emitting array 1101 does not operate unless the apparatus is within the mouth of the patient. This configuration can also be useful for obtaining information about a patient's bone density, where the information can be useful for customizing a dosage of light therapy to be administered to a patient, as described herein. The foregoing compliance assessment mechanism can be included in, or otherwise incorporated into, any apparatus for intra-oral light therapy described herein.

In another example, referring to FIG. 40B, an apparatus according to an embodiment is configured to detect an amount of light, e.g., its intensity or duration, that is irradiated at, absorbed by or reflected by a patient's periodontia (e.g., a portion of the root area of the upper and/or lower jaw and/or the alveolar soft tissue) by the apparatus. For example, the apparatus can be configured to determine an energy density that is irradiated at, absorbed by or reflected by the root area from an amount or dosage of light to which the root area was exposed. The apparatus is also configured to determine whether an amount of light being emitted by the apparatus should be adjusted based on the detected amount, intensity and/or duration of light (or energy density) is irradiated at, absorbed by or reflected by the patient's periodontia, as described herein.

In one or more embodiments, the apparatus includes a mouthpiece having a first flange 1110 and a second flange 1116. The first flange 1110 includes one or a plurality of light emitters 1111, and is configured to be disposed adjacent the buccal side of a first portion of the root area of the upper and/or lower jaw and/or the alveolar soft tissue (generally designated as tissue T) when the mouthpiece is disposed within the patient's oral cavity. In one or more embodiments, the one or plurality of light emitters 1111 can be at least partially or wholly enclosed in the first flange. In one or more embodiments, the one or plurality of light emitters 1111 is disposed on a surface of the first flange. The one or plurality of light emitters 1111 are positioned such that light emitted therefrom is directed to the first portion of the root area of the upper and/or lower jaw and/or the alveolar soft tissue. The one or plurality of light emitters 1111 is configured to be in electrical communication with a controller 1114, such as via pathway 1113. In this manner, the controller 1114 can control parameters (e.g., duration, intensity, and wavelength) affecting the emission of light by the one or plurality of light emitters 1111.

The second flange 1116 of the mouthpiece includes one or more photodetectors 1112, and is configured to be disposed adjacent the palatial or lingual side of a second portion, opposite to the first portion, of the root area of the upper and/or lower jaw and/or the alveolar soft tissue when the mouthpiece is disposed within the patient's oral cavity (and the first flange is disposed adjacent the buccal side of the first portion of the root area of the upper and/or lower jaw and/or the alveolar soft tissue). The photodetector 1112 can be at least partially or wholly enclosed within the second flange 1116. The photodetector 1112 is configured to receive light passed through the root area of the upper and/or lower jaw and/or the alveolar soft tissue between the first portion and the second portion. The photodetector 1112 is configured to be in electrical communication with the controller 1114, such as via pathway 1118. The photodetector 1112 is configured to convey information associated with the light received by the photodetector 1112 to the controller 1114. For example, the photodetector 1112 can convey information to the controller 1114 associated with the intensity of light received.

The controller 1114 is configured to execute an algorithm for determining whether a parameter of light emission by the one or plurality of light emitters 1111 should be adjusted, for example, to achieve a target light transmission through the patient's tissue. For example, the controller 1114 can execute the algorithm based on the information associated with the light received by the photodetector 1112 and conveyed to the controller 1114, as well as one or more known parameters (e.g., duration, intensity, and wavelength) associated with the light emission by the one or plurality of light emitters 1111. The controller 1114 can be configured to adjust one or more parameters of light emission by the one or plurality of light emitters 1111 based on the foregoing determination. The parameters of light emission that can be adjusted, or otherwise controlled, by the controller 1114 include an intensity of light emitted by the one or plurality of emitters 1111, a duration of emission of light by the one or plurality of light emitters 1111, one or more wavelengths of light, or one or more of the intensity, duration, and wavelength.

In one or more embodiments, the controller 1114 is configured to determine whether the mouthpiece of the apparatus is positioned with respect to (e.g., adjacent) the maxilla or mandible root areas. For example, the controller 1114 can cause the one or plurality of light emitters 1111 to emit light at a known intensity, duration, or wavelength. The controller 1114 can then receive information from the photodetector 1112 associated with the transmission of light through the root area, and then determine whether the light was transmitted through the maxillary root area or the mandibular root area based on the received information. In other words, the controller 1114 can determine whether the mouthpiece was positioned with respect to the maxilla if the light transmission received by the photodetector 1112 is within a first value range, or whether the mouthpiece was positioned with respect to the mandible if the light transmission received by the photodetector 1112 is within a second value range.

In one or more embodiments, the apparatus is configured to be calibrated prior to or at the beginning of a prescribed treatment regime with respect to each of the mandible and maxilla. In this manner, the mouthpiece is positioned with respect to the maxilla, then light is emitted by the one or plurality of light emitters 1111 and an energy density based on the light transmitted through the maxillary root area is detected by the photodetector 1112. With respect to the maxilla, the value of light transmission or reflection (as the case may be), referred to herein as the Iᵣₐₜᵢₒ, can be calculated as follows, with I_{delivery} being the value (e.g., intensity measured in mW/cm²) of light emitted by the emitter and Iₜᵣₐₙₛₘᵢₛₛᵢₒₙ being the value (e.g., intensity measured in mW/cm²) of light received by the photodetector:

$$\frac{{I}_{delivery ( max )} mW / {cm}^{2}}{{I}_{transmission ( max )} mW / {cm}^{2}} = {γ}_{1 max} mW / {cm}^{2}$$

Similarly, with respect to the mandible, the Iᵣₐₜᵢₒ, can be calculated as follows:

$$\frac{{I}_{delivery ( mnd )} mW / {cm}^{2}}{{I}_{transmission ( mnd )} mW / {cm}^{2}} = {γ}_{1 mnd} mW / {cm}^{2}$$

The Iᵣₐₜᵢₒ can be, for example, based at least in part on photon power density. The controller 1114 can be configured to store an Iᵣₐₜᵢₒ value (i.e., the γ₁ₘₐₓ and/or γ_{1mnd}). In this manner, the apparatus can reference the stored values to determine whether the mouthpiece is optimally positioned with respect to the maxilla or mandible. In a similar manner as described herein with reference to FIG. 40A, the apparatus can be configured to monitor patient compliance throughout the duration of an orthodontic treatment regime. In use, each Iᵣₐₜᵢₒ value can be adjusted based on a patient's range of tolerance according to the following calculation: γ1% range of tolerance. In one or more embodiments, the controller 1114 is configured to adjust the I_{delivery} to achieve a desired Iₜᵣₐₙₛₘᵢₛₛᵢₒₙ, such as by selectively changing (e.g., increasing or decreasing) the I_{delivery} intensity.

Although the Iᵣₐₜᵢₒ is described herein as being measured in mW/cm², in one or more embodiments, the Iᵣₐₜᵢₒ can be measured using a different unit of measurement commensurate with a desired lighting parameter, or characteristic. For example, the Iᵣₐₜᵢₒ can be measured with respect to light wavelength (in, e.g., nanometers). In this manner, the controller 1114 can be configured to, for example, analyze the cellular photo-absorption state as represented by changes in wavelengths absorbed and/or transmitted by chromophores in the patient's tissue.

Referring to FIG. 41, an apparatus according to an embodiment is configured to control an amount, intensity, wavelength and/or duration of light emitted towards a portion of the root area of the upper and/or lower jaw and/or the alveolar soft tissue and to detect an amount, intensity, wavelength and/or duration of light passed through the root area of the upper and/or lower jaw and/or the alveolar soft tissue. The apparatus can be similar in many respects, or identical to, or include components similar in many respects, or identical, to components of, any other apparatus described herein, such as, for example, apparatus described herein with reference to FIGS. 40A and 40B. In one or more embodiments, the apparatus includes a mouthpiece having a first flange 1120 configured to be disposed on the buccal side of the root area and a second flange 1126 configured to be disposed on the palatial or lingual side of the root area.

One or more light emitters (e.g., a plurality of light emitters) 1121 are disposed in the first flange 1120. The one or more light emitters 1121 includes individually addressable (or controllable) sections 1123. Parameters affecting the emission of light (e.g., intensity, duration and/or wavelength) by a section of the one or more light emitters 1121 can be controlled separately from and independently of a different section of the one or more light emitters. Stated another way, the intensity, duration, and/or wavelength of light emitted by the one or more light emitters 1121 can vary among the various sections 1123 of the one or more light emitters. The second flange 1126 includes one or more photodetectors (e.g., a plurality of photodetectors 1122). The plurality of photodetectors 1122 can include two or more discrete photodetectors 1124. In one or more embodiments, the one or more photodetectors 1122 includes a number of photodetectors 1124 equal to the number of sections 1123 in the one or more light emitters 1121. In this manner, each photodetector 1124 can be configured to receive light that was emitted by a corresponding section 1123 of the one or more light emitters 1121 and that passed through the root area between the section 1123 and the photodetector 1124. In this manner, light emission by each section of the one or more light emitters 1121 can be adjusted to accommodate variations in treatment goals for and/or anatomy of different patients, whose alveolar dimensions can vary. These adjustments can be based at least in part on the light received by the corresponding photodetector 1124.

In one or more embodiments, an apparatus including photodetectors for sensing light transmission or reflection, such as the apparatus described herein with reference to FIGS. 40A-41, is configured to perform an initial calibration. For example, in one or more embodiments, to calibrate the apparatus, the patient places the apparatus in the upper arch of the oral cavity. The apparatus registers its orientation (i.e., upright or upside down) using an internal orientation-sensing mechanism (e.g., a gyroscope). A light source, such as one or more LEDs, is activated. One or more optical fibers act as receivers and photodetectors at their distal ends, which are configured to determine photon, or light, transmission or reflection. Activation of the light source and determination of the light transmission or reflection by the receiver/photodetector fibers is repeated, such as for two or three times. An average value of light transmission or reflection is calculated, and the calculated average value can be useful as a threshold or range during the orthodontic treatment. If the amount of light detected becomes inconsistent with the threshold or range, then the apparatus deactivates to stop the orthodontic treatment.

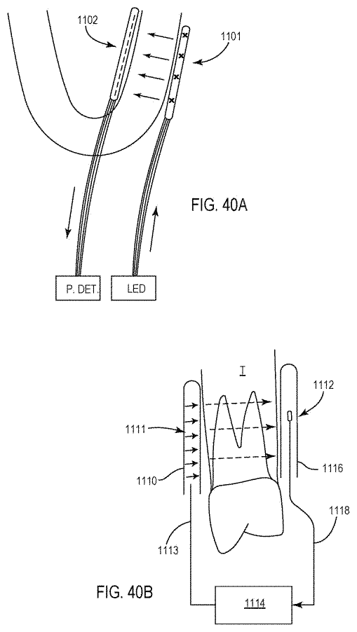

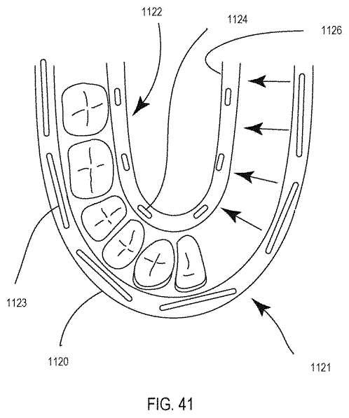

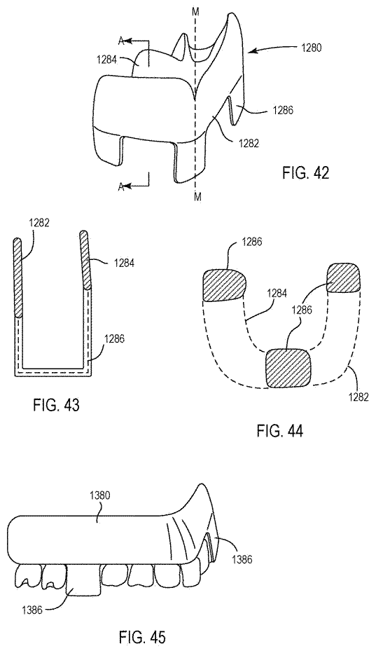

In one or more embodiments, an apparatus includes an intra-oral housing that is contoured to complement curvature and/or other physical attributes of a patient's tissue within the patient's oral cavity. For example, referring to FIGS. 42-44, an apparatus according to an embodiment includes an intra-oral housing 1280 including a front portion 1282 configured to be disposed adjacent buccal alveolar soft tissue of a patient and a rear portion 1284 configured to be disposed adjacent lingual alveolar soft tissue of the patient. A midline M divides left and right sides (also referred to herein as wings or flanges) of the front and rear portions 1282, 1284, respectively, of the intra-oral housing 1280. In one or more embodiments, a height of the left and or right side is configured to correspond to a length including an average incisor root length and a length of the premolars. A portion of the side that is configured to be adjacent the canine teeth can be slightly under-extended compared to, or shorter than, a different (e.g., incisor) portion of the side. A first light emitting array or mat (not shown in FIGS. 42-44) configured to be disposed adjacent the buccal alveolar soft tissue, for example, can have a length substantially equal to a width of two molar teeth. A second, corresponding light emitting array or mat configured to be disposed on the palatial side of the teeth, adjacent the lingual alveolar soft tissue for example, can have a similar length as the first light emitting array. The intra-oral housing 1280 can include a palatial portion or wing 1288, which can be configured to move vertically with respect to the first and second sides, e.g., up against the upper hard palate.

The intra-oral housing 1280 includes one or more distinct segments 1286 that each include a first portion extending (e.g., downwardly) from a lower surface of the front portion 1282 of the intra-oral housing, a second portion extending (e.g., downwardly) from a lower surface of the rear portion 1284 of the intra-oral housing, and a third portion extending (e.g., horizontally) between ends of the first portion and the second portion of the segment 1286. In this manner, the segments 1286 (also referred to as bite pads) are configured to be disposed about at least a portion of crowns of one or more teeth adjacent each segment when the intra-oral housing 1280 is disposed in the oral cavity as described herein. The segments 1286 are laterally spaced apart from each other with respect to the front portion 1282 of the intra-oral housing 1280. In this manner, when the intra-oral housing 1280 is disposed in the oral cavity as described herein, the segments 1286, or bite pads, are disposed about the crowns of fewer than all teeth. In one or more embodiments, a first number of the patient's teeth are covered by the segments 1286 and a second number, greater than the first number, are not covered by the segments. For example, each segment 1286 can have a sufficient height, width, and/or depth for being disposed about the crowns of one or two teeth. In use, the patient can bite down on the segments 1286 during the orthodontic treatment, such as to maintain a position of the intra-oral housing 1280 within the oral cavity. Use of the segments, or bite pads, described herein also serves to reduce bulk associated with the surface area of the apparatus, and thus provides enhanced patient comfort.

The segments 1286 can be constructed of a material similar or identical to or different than that of the front and rear portions 1282, 1284 of the intra-oral housing 1280. For example, the front and rear portions 1282, 1284 of the intra-oral housing 1280 can be constructed of a soft, e.g., malleable, material such as silicone, and the segments 1286 can be constructed of a harder, less malleable material, e.g., silicone, that is overmolded with a soft material, such as the soft silicone.

In the embodiment illustrated in FIGS. 42-44, the intra-oral housing 1280 includes three bite pad segments. The first segment is extended from the lower surfaces of the front and rear portions 1282, 1284 along the midline M. The second and third segments are extended from the lower surfaces of the front and rear portions 1282, 1284 at ends of the left and right sides of the intra-oral housing 1280 opposite the midline M. In other embodiments, however, the segments can extend from a different location along the lower surfaces of the front and rear portions 1282, 1284. For example, as shown in FIG. 45, an apparatus can include an intra-oral housing 1380 including a segment extending from the lower surfaces of front and rear portions of the intra-oral housing at a midline (not shown in FIG. 45) defined by the intra-oral housing, and one or more segments extending from the lower surfaces of the front and rear portions of the intra-oral housing from one or more locations between the midline and left and right ends, respectively, of the intra-oral housing opposite the midline.

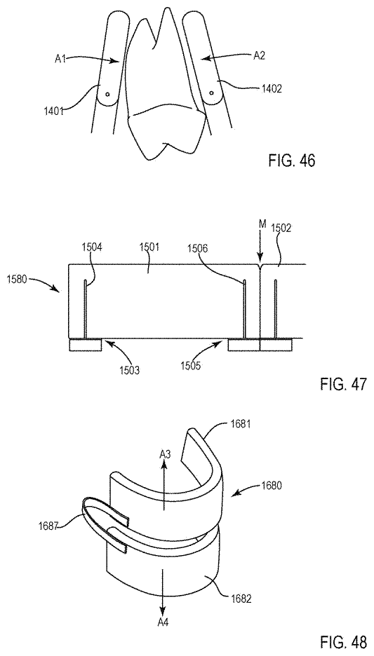

In one or more embodiments, at least a portion of an apparatus is biased towards a portion of the patient's body, which can, for example, help maintain the position of the apparatus with respect to a patient's oral cavity. As shown in FIG. 46, an apparatus can include a first portion 1401 configured to be disposed adjacent a root area of the jaw between the root area and the buccal mucosa, and a second portion 1402 configured to be disposed adjacent the palatial side of the root area of the jaw. The first portion 1401 is biased in a first direction towards the root area, and the second portion 1402 is biased in a second, opposite direction towards the root area. More specifically, the first portion 1401 is spring-loaded such that a free end of the first portion is moved toward and/or can apply a pressure upon the root area in the first direction, indicated by arrow A1, and the second portion 1402 is spring-loaded such that a free end of the second portion is moved toward and/or can apply a pressure upon the root area in the second direction, indicated by arrow A2. In one or more embodiments, the pressure applied by the first portion 1401 and/or the second portion 1402 is sufficient to displace at least a portion of the patient's tissue. The first and second portions 1401, 1402 can be configured to pivot at a joint disposed adjacent the root area, e.g., immediately above the crown of the tooth.

In another example, as shown in FIG. 47, an apparatus can include an intra-oral housing 1580 having a right flange 1501 and a left flange 1502 divided by a midline M. The right flange 1501 and left flange 1502 are each configured to be disposed adjacent a root area of the jaw. The right flange 1501 is biased in a first direction towards the root area. The right flange 1501 is coupled to the apparatus by one or more hinges (e.g., two hinges 1503, 1505, as shown in FIG. 47) and includes one or more wires (e.g., two nitinol or other super-elastic wires, each associated with a respective hinge, as shown in FIG. 47) embedded within the right flange 1501 of the intra-oral housing 1580. For example, the hinges 1503, 1505 can move about a horizontal axis and the nitinol wires 1504, 1506 can be embedded in a silicone of the intra-oral housing 1580 along an axis substantially normal to the horizontal axis of the hinges, e.g., plus or minus 5 degrees of the normal to the horizontal axis. The wires 1504, 1506 are configured to be biased towards the root area. As such, the wires 1504, 1506 push against an apical portion of the right flange and cause the right flange 1501 to push against the tissue of the root area. The left flange 1502 can be configured similarly to the right flange 1501. In one or more embodiments, the hinges 1503, 1505 and/or wires 1504, 1506 can produce a relatively large orthodontic and/or orthopedic force, such as a force operable to urge one or more teeth to move.

In another example, an apparatus according to an embodiment is illustrated in FIG. 48. The apparatus includes an intra-oral housing 1680 having an upper portion 1681 extended between a first end and a second end, and a lower portion 1682 extended between a first end and a second end. The upper and lower portions 1681, 1682 are each configured to be disposed adjacent a root area within an oral cavity. The upper and lower portions 1681, 1682 have a curvature configured to correspond to a curvature of a patient's dentition (e.g., is U or horseshoe shaped). The first end of the upper portion 1681 is coupled to the first end of the lower portion 1682 by a shape retaining member 1687. The shape retaining member 1687 can include, for example, a curved stiff plastic material having shape memory characteristics. The shape retaining member 1687 is biased towards an open configuration in which the first end of the upper portion 1681 is moved in a first direction, indicated by arrow A3, away from the first end of the lower portion 1682 of the intra-oral housing 1680, and the first end of the lower portion 1682 is moved in a second, opposite direction, indicated by arrow A4, away from the first end portion of the upper portion 1681 of the intra-oral housing 1680. The second end of the upper portion 1681 is coupled to the second end of the lower portion 1682 by a similar shape retaining member 1687. As such, the second end of the upper portion 1681 and second end of the lower portion 1682 are biased away from each other towards the open configuration. In this manner, the upper and lower portions 1681, 1682 of the intra-oral housing 1680 are configured to remain adjacent their respective root portions when the patient jaws are opened. The biasing force exerted by the shape retaining member 1687 is sufficient to move the seating of at least a portion of the upper portion 1681 and/or lower portion 1682 toward a depth of the patient's cheek/alveolar mucosa vestibule; such force alone, however, might be insufficient to cause the jaw of the patient to open.

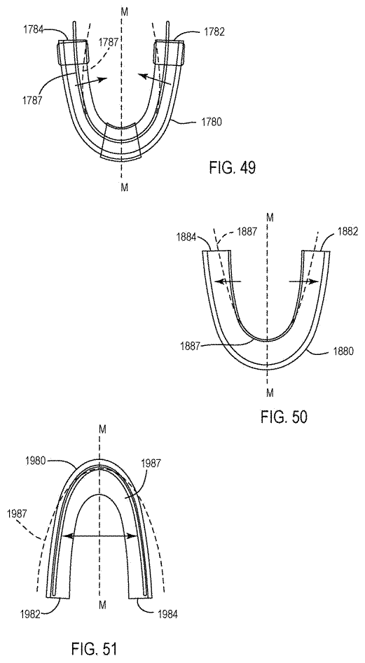

A portion of an apparatus according to an embodiment is illustrated in FIG. 49. The apparatus includes a substantially U or horseshoe shaped intra-oral housing, e.g., has a curvature approximating the curvature of a U or horseshoe shape. The intra-oral housing can be constructed of a soft silicone. A wire 1787 (e.g., nitinol or other super-elastic wire), or other shape retaining member, is embedded in the intra-oral housing. A first end of the wire is disposed at a first end 1782 of the intra-oral housing 1780, and a second end of the wire is disposed at a second, opposite, end 1784 of the intra-oral housing. The first and second ends of the wire are inwardly biased such that the first end of the intra-oral housing and second end of the intra-oral housing are (or can be) moved in a direction towards each other. In other words, the wire is biased to move from an open position in which the first and second ends 1782, 1784 of the intra-oral housing 1780 are disposed a first distance from a midline M of the intra-oral housing, as shown by the solid line in FIG. 49, to a closed position in which the first and second ends 1782, 1784 are disposed a second distance less than the first distance from the midline M of the intra-oral housing, as shown by the dashed line in FIG. 49. In this manner, the wire causes a portion of the intra-oral housing (e.g., left and right portions and/or light emitting panels) to apply a gentle pressure on the buccal side of the root area towards a lingual or palatial side of the root area. Also in this manner, the intra-oral housing 1780 is configured to cause displacement of a portion of oral soft tissue over the tooth root.

In one or more embodiments, as shown in FIG. 50, an apparatus can include an intra-oral housing 1880 having a wire 1887 (e.g., nitinol or other super-elastic wire), or other shape retaining member, configured to bias first and second ends 1882, 1884, respectively, of the intra-oral housing 1880 away from each other, e.g., in a direction opposite to that shown and described with reference to FIG. 49. In other words, the wire 1887 is biased to move from a closed position in which the first and second ends 1882, 1884 are disposed a first distance from a midline M of the intra-oral housing as shown by the solid line in FIG. 50 to an open position in which the first and second ends 1882, 1884 are disposed a second distance greater than the first distance from the midline M of the intra-oral housing as shown by the dashed line in FIG. 50. In this manner, the wire is configured to cause a portion of the intra-oral housing (e.g., left and right portions and/or light emitting panels, not shown in FIG. 50) to apply a gentle pressure on the lingual or palatial side of the root area towards, or in the direction of, the buccal side of the root area. Also in this manner, the intra-oral housing 1880 is configured to cause displacement of a portion of oral soft tissue over the tooth root.

The wire 1887 can be disposed within the intra-oral housing 1880 in any suitable position. For example, as shown in FIG. 50, the wire 1887 can be positioned adjacent an inner curvature of the intra-oral housing 1880. In other embodiments, however, a wire 1987 can be positioned adjacent an outer curvature of an intra-oral housing 1980, as shown in FIG. 51. The wire 1987 can be similar in many respects to wire 1887, for example, in that the wire 1987 can be configured to bias first and second ends 1982, 1984, respectively, of the intra-oral housing 1980 away from each other, e.g., in a direction opposite to that shown and described with reference to FIG. 49 and similar to that shown and described with reference to FIG. 50. In other words, the wire 1987 is biased to move from a closed position (as shown by the solid line in FIG. 51) to an open position (as shown by the dashed line in FIG. 50) in a similar manner as described herein with reference to FIG. 50. Thus, the wire 1987 is configured to cause a portion of the intra-oral housing 1980 to apply a gentle laterally, and outwardly, directly pressure on the lingual or palatial side of the root area.

Although the apparatuses depicted in FIGS. 49-51 are described as including a wire (e.g., a nitinol wire) as the shape retaining member, in other embodiments, a different shape retaining member (or biasing member) can be included in the apparatus. For example, the shape retaining member can include an overmolded plastic insert.

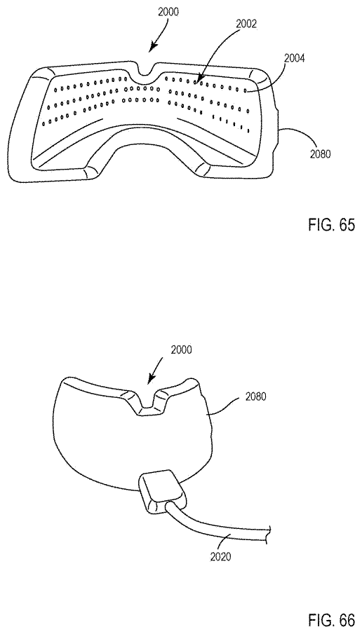

An intra-oral apparatus 2000 according to an embodiment of the invention is illustrated in FIGS. 65 and 66. The apparatus 2000 can be the same as or similar in many respects to, or include components the same as or similar in many respects to, the intra-oral apparatuses described herein. The intra-oral apparatus 2000 includes an intra-oral housing 2080 configured to be disposed in an oral cavity of a patient. In one or more embodiments, the intra-oral housing 2080 is configured to be positioned within the oral cavity of the patient with respect to the upper jaw, the lower jaw, or each of the upper and lower jaws. The intra-oral housing 2080 includes at least one light emitting panel 2002. The light emitting panel 2002 can include one or more light emitters 2004, such as LEDs. The intra-oral apparatus 2000 can be configured to irradiate light in any suitable manner described herein. In one or more embodiments, for example, the intra-oral apparatus 2000 can be configured to irradiate light at a density of about 60 mW/cm².

Although the light emitting panel 2002 is illustrated as including the one or more light emitters 2004 in three parallel rows, in other embodiments, the one or more light emitters can be differently positioned with respect to the light emitting panel and/or the intra-oral housing (e.g., in one or more vertical rows, one or more diagonal rows, a random pattern, or any other suitable configuration). In one or more embodiments, the light emitting panel 2002 is at least partially enclosed within the intra-oral housing 2080. For example, the light emitting panel 2002 can be embedded within the intra-oral housing 2080. The intra-oral housing 2080 can be constructed of any suitable material, including, for example, a soft silicone material. The intra-oral housing 2080 is configured to be electrically coupled to an electronic device, such as a controller (not shown in FIGS. 65-66) as described herein. As shown in FIG. 66, the intra-oral housing 2080 can be coupled to the electronic device by a tether 2020.

The intra-oral apparatus 2000 can be configured for use in an orthodontic treatment, including any such treatment described herein. In one or more embodiments, for example, the intra-oral apparatus 2000 is useful for irradiating at least a portion of the patient's upper jaw for about 3 minutes, the patient's lower jaw for about 3 minutes, or each of the patient's upper and lower jaws for about 3 minutes.

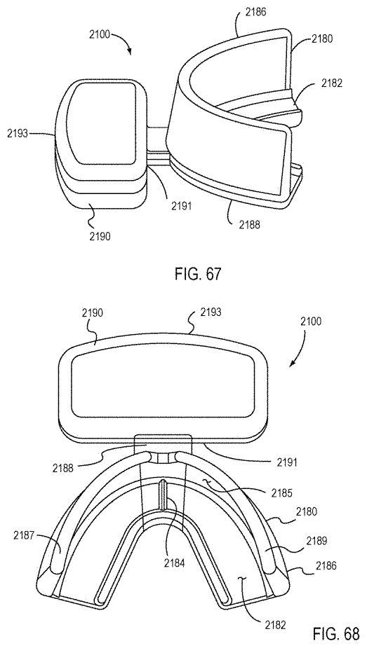

An intra-oral apparatus 2100 according to an embodiment of the invention is illustrated in FIGS. 67-72. The apparatus 2100 can be the same as or similar in many respects to, or include components the same as or similar in many respects to, the intra-oral apparatuses described herein. The intra-oral apparatus 2100 includes an intra-oral housing 2180 configured to be disposed in an oral cavity (e.g., in the mouth) of a patient and an extra-oral housing 2190 (also referred to herein as a “bill”) coupled to the intra-oral housing 2180. The extra-oral housing 2190 is coupled to a front portion of the intra-oral housing 2180. For example, the extra-oral housing 2190 can be coupled to the intra-oral housing 2180 by a protrusion 2188. In this manner, the protrusion 2188 is extended through the opening of the patient's mouth, e.g., through the opening between the patient's lips, such that at least a portion of the extra-oral housing 2190 is disposed exterior to the oral cavity of the patient when the intra-oral housing 2180 is disposed within the oral cavity of the patient. Also in this manner, the extra-oral housing 2190, or bill, can be supported with respect to the patient's mouth by the intra-oral housing 2180 and/or the protrusion 2188 when the intra-oral housing 2180 is disposed within the patient's mouth. The extra-oral housing 2190 is configured to at least partially enclose one or more electronic components of the apparatus 2100, as described in more detail herein.

The intra-oral apparatus 2100 is configured to be useful for light therapy with respect to each of the upper jaw and the lower jaw of the patient. In other words, the intra-oral apparatus 2100 can be configured to administer light therapy with respect to the patient's upper jaw when the apparatus is in an upright position, and can be configured to administer light therapy with respect to the patient's lower jaw when the apparatus is in an inverted position. As such, the intra-oral housing 2180 can be configured to be disposed within the patient's oral cavity with respect to each of the upper and lower jaws of the patient. It should be noted that although the intra-oral apparatus 2100 and intra-oral housing 2180 are described as being in the upright position when configured to be oriented with respect to the upper jaw and in the inverted position when configured to be oriented with respect to the lower jaw, in other embodiments, the intra-oral apparatus 2100 and the intra-oral housing 2180 are in the upright position when configured to be oriented with respect to the lower jaw of the patient, and in the inverted position when configured to be oriented with respect to the upper jaw of the patient.

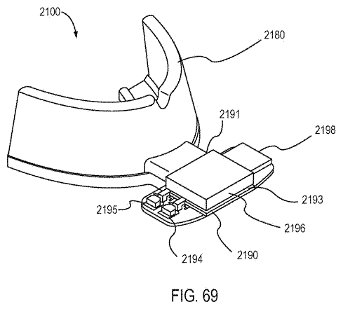

The intra-oral apparatus 2100 can be configured to determine the orientation of the apparatus. Stated another way, the intra-oral apparatus 2100 can be configured to determine if the intra-oral housing 2180 is oriented in the upright or inverted position. For example, the intra-oral apparatus 2100 includes a gyroscope (not shown in FIG. 69) configured to determine if the intra-oral housing 2180 is oriented in the upright or inverted position. The gyroscope is disposed within, or otherwise coupled to, the extra-oral housing 2190 of the apparatus 2100.

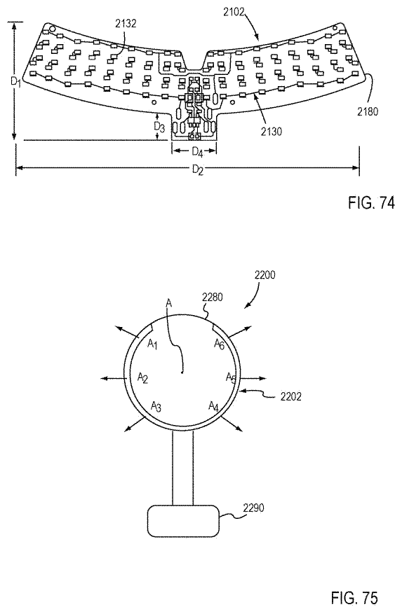

The apparatus 2100 includes at least one battery, or other suitable power source. For example, as shown in FIG. 69, a first battery 2194 and a second battery 2195 are coupled to the extra-oral housing 2190 of the apparatus 2100. For example, the batteries 2194, 2195 can be disposed in the extra-oral housing 2190. Each battery 2194, 2195 can be configured to provide power to one or more light-emitting panels (schematically illustrated in FIG. 74) disposed within the intra-oral housing 2180, as described in more detail herein. The first battery 2194 can include, for example, a rechargeable lithium ion battery. Alternatively or in addition, apparatus 2100 can comprise a charging station that is configured for inductive charging (e.g., to power the apparatus 2100 and/or to charge a battery thereof). For example, the apparatus 2100 can comprise a Qi-based charging coil.

A microprocessor 2196 is coupled to the extra-oral housing 2190 of the apparatus 2100. The microprocessor 2196 can be disposed in the extra-oral housing 2190. The microprocessor 2196 is configured to store information related to the patient's use of the intra-oral apparatus 2100. For example, the microprocessor 2196 can be configured to store information associated with the patient's treatment program and use of the apparatus 2100 during the treatment program, including, for example, a schedule of one or more treatment sessions included in the treatment program, an orientation of the apparatus 2100 during a treatment session, a duration of a treatment session, and a duration between a treatment session and one or more previous treatment sessions. The microprocessor 2196 can also be configured to determine whether the patient's usage of the intra-oral apparatus 2100 is in compliance with the patient's treatment program. In other words, the microprocessor 2196 can be configured to determine whether a patient's history of use (including, for example, a number of treatment sessions applied to the upper and/or lower jaw of the patient, duration of the treatment sessions, whether any treatment session was interrupted, and the like) complies with a schedule of treatment sessions specified by the patient's treatment program, including identifying any deviation from the treatment program. The microprocessor's 2196 determination regarding patient compliance can be based, at least in part, on information received from the proximity detector. For example, the proximity detector can be configured to be activated when the apparatus is placed fully into the patient's mouth. The microprocessor 2196 can be configured to transmit information associated with the patient's usage and/or compliance of the apparatus 2100 with an external device. For example, in one or more embodiments, the microprocessor 2196 is configured to transmit the usage and/or compliance information to the external device (e.g., a mobile phone, personal digital assistant, computer, portable electronic device, or the like) via Bluetooth® or another suitable wireless mechanism. For example, as shown in FIG. 69, a Bluetooth® communication module 2198 can be disposed within the extra-oral housing 2190.

The extra-oral housing 2190 includes a communication mechanism (not shown in FIG. 69) configured to provide indicia of the status of a treatment session. The term “indicia,” is used herein as including the singular (“indicium”) or the plural (“indicia”), unless the context clearly indicates otherwise. The indicia can include one or more of an audible indicia (e.g., a tone, beep, announcement, or the like), a tactile indicia (e.g., a vibration or the like), or a visual indicia (e.g., a light, a displayed message, or the like). More specifically, for example, the extra-oral housing 2190 includes a light indicia that is configured to indicate a status, or stage, of the treatment session. The light indicia is configured to display no light during a first stage of the treatment session, a blinking or pulsed light during a second stage of the treatment session, and/or a solid light during a third stage of the treatment session. The light indicia can be, for example, configured to display a solid light for a first predetermined duration (e.g., 2 minutes and 30 seconds, 2 minutes and 45 seconds, or 2 minutes and 10 seconds) upon initiation of the treatment session. The light indicia can be configured to display the blinking or pulsed light for a second predetermined duration (e.g., 10, 15 or 30 seconds) following the first predetermined duration as a signal to the patient that the treatment session is nearing its end. The light indicia can be configured to display no light when a treatment session is ended (e.g., after 3 minutes from initiation of the treatment session) and the apparatus 2100 is not irradiating light.

In one or more embodiments, the extra-oral housing 2190 has a sufficient length (e.g., between a first end 2191 of the extra-oral housing engaged with the intra-oral housing 2080 and a second, opposite, end 2193 of the extra-oral housing (i.e., the end of the extra-oral housing farthest from the patient's oral cavity when the intra-oral housing is disposed in the patient's oral cavity)) such that at least a portion of the extra-oral housing is visible to the patient when the intra-oral housing is disposed in the patient's oral cavity. In other words, at least a portion of the extra-oral housing 2190, e.g., including the second end 2193 of the extra-oral housing, is within the patient's line of sight when the intra-oral housing 2180 is disposed with the patient's oral cavity. In this manner, the light indicia can be coupled to the extra-oral housing 2190 in a manner such that the light indicia is within the patient's line of sight during the treatment session.

As noted herein, the intra-oral housing 2180 is configured to be positioned within the oral cavity of the patient with respect to the upper jaw, the lower jaw, or is invertible for positioning with respect to each of the upper and lower jaws. The intra-oral housing 2180 can be similar in one or more respects, and include components similar in one or more respects, or identical, to the intra-oral housings described herein, including, for example, the intra-oral housings described herein with reference to FIGS. 35-37 and 65-66. Accordingly, the intra-oral housing 2180 is not described in detail.

The intra-oral housing 2180 includes a lower portion 2182 and an upper portion 2186. The lower portion 2182 has a first plane, and the upper portion 2186 has a second plane different than the first plane. For example, the upper portion 2186 can be substantially vertical (e.g., plus or minus about 5 degrees from the vertical axis or plane), and the lower portion 2182 can be substantially horizontal (e.g., plus or minus about 5 degrees from the horizontal axis or plane) when the intra-oral housing 2180 is disposed within the patient's oral cavity for a treatment session. In this manner, the upper portion 2186 can be disposed adjacent a portion of a side of the patient's teeth and/or adjacent the alveolar mucosa and the lower portion 2182 can be disposed adjacent an occlusal surface of the patient's teeth. For example, the lower portion 2182 can be configured as a bite pad for the patient to bite down upon during a treatment session.

As shown in FIG. 68, the lower portion 2182 of the intra-oral housing 2180 includes a ridge 2184. The ridge 2184 is disposed along a midline of the intra-oral housing 2180 and is elevated with respect to the first plane of the lower portion 2182. The ridge 2184 facilitates positioning of the intra-oral housing 2180 within the patient's oral cavity by the patient when the intra-oral housing 2180 is disposed within the patient's oral cavity. For example, the intra-oral housing 2180 is configured to be positioned within the patient's oral cavity such that the ridge 2184 is disposed between the patient's front central incisors. Proprioception of the patient related to the teeth and periodontium would permit sensory feedback to the patient regarding the position of the ridge 2184 of the intra-oral housing 2180. In this manner, the ridge 2184 facilitates centering of the intra-oral housing 2180 within the oral cavity, thus promoting symmetry of a light therapy treatment on the alveolar mucosa, or other oral tissue, on both sides of the patient's mouth. In other words, in order to promote the symmetrical administration of light therapy to the root area, the intra-oral housing 2180 can be positioned with the midline of the intra-oral housing 2180 seated along the sagittal plane or within (i.e., plus or minus) 5 degrees of the sagittal plane, and the ridge 2184 can facilitate such obtaining such a position.

The upper portion 2186 includes a first (or left) flange 2187 and a second (or right) flange 2189. The flanges 2187, 2189 are each configured to apically displace oral soft tissue. More specifically, the flanges 2187, 2189 are each configured to displace buccal tissue away from the patient's alveolar mucosa. In one or more embodiments, an inner face 2185 of the upper portion 2186 can be spaced apart from the patient's alveolar tissue when the intra-oral housing 2180 is disposed within the patient's mouth and the flanges 2187, 2189 are displacing the buccal tissue. In one or more embodiments, at least a portion of the inner face 2185 of the upper portion 2186 can contact the patient's alveolar tissue when the intra-oral housing 2180 is disposed within the patient's mouth and the flanges 2187, 2189 are displacing the buccal tissue.

The intra-oral housing 2180 can be constructed of any suitable material, including, for example, an elastomeric material (e.g., a soft silicone). More specifically, in one or more embodiments, the intra-oral housing can be fabricated from medical-grade injection molded highly flexible silicone. The ridge 2184 can be constructed of the same material as the intra-oral housing 2180, or at least the same material as the lower portion 2182 of the intra-oral housing 2180. In this manner, when a patient bites together with the upper and lower jaw, the lower portion 2182 of the intra-oral housing 2180, including the ridge 2184, can deform slightly from pressure exerted by an occlusal surface of the patient's teeth. Nonetheless, the ridge 2184 is of sufficient dimensions that the patient should be aware of its position, despite any slight deformation of the lower portion 2182 and/or ridge 2184.

The intra-oral housing 2180 includes at least one light emitting panel 2102 (the circuitry 2130 of which is schematically illustrated in FIG. 74). The light emitting panel 2102 can include one or more light emitters 2132, such as a plurality of LEDs, and a flexible circuit 2130. The intra-oral apparatus 2100 can be configured to irradiate light in any suitable manner described herein to irradiate the alveolar mucosa and/or root area of the patient. The LEDs, or other suitable light emitter(s), can be included in the light emitting panel 2102 in any suitable configuration, including in any configuration described herein. In one or more embodiments, the light emitting panel 2102 is at least partially enclosed within the intra-oral housing 2180. For example, the light emitting panel can be embedded within the intra-oral housing 2180 (e.g., in the inner face 2185 of the upper portion 2186 of the intra-oral housing 2180. As noted herein, the intra-oral housing 2180 can be constructed of a soft silicone material. In this manner, the light emitting panel, and thus any LED or light emitter included in the panel, can be embedded in the silicone material such that the light emitting panel is prevented from engaging a portion of the patient's oral tissue when the intra-oral housing 2180 is disposed within the patient's oral cavity. The light emitting panel 2102 can have any suitable dimensions for being coupled to, or embedded in, the upper portion 2186 of the intra-oral housing 2180. For example, as shown in FIG. 74, the light emitting panel 2102 can have a height D1 and a width D2 greater than the height D1. In one or more embodiments, for example, the panel 2102 has a height of about 31.9 mm and a width of about 92.5 mm. A portion of the height D1 of the panel 2102 can include a lower protrusion that extends downwardly from left and right flanges 2187, 2189 of the intra-oral housing 2180. The protrusion can have a height D3 and a width D4. In one or more embodiments, for example, the protrusion has a height of about 6.9 mm and a width of about 12 mm. Although specific examples of the dimensions of the panel 2102 are provided, such dimensions are presented by way of example only, and not limitation.

The intra-oral housing 2180 can be configured to be disposed within the patient's oral cavity such that an outer surface of the intra-oral housing 2180 is spaced apart from the alveolar soft tissue of the patient. In this manner, the intra-oral housing 2180 is configured to be spaced apart from (i.e., not touch) the alveolar soft tissue of the patient during the treatment session. In one or more embodiments, for example, at least a portion of the intra-oral housing 2180 can be configured to be disposed over at least a portion of the patient's teeth. A first portion of the intra-oral housing 2180 is disposed about the portion of the patient's teeth and a second portion of the intra-oral housing 2180 is disposed proximate to and spaced apart from the alveolar soft tissue when the intra-oral housing 2180 is disposed in the patient's mouth.

In one or more embodiments, at least a portion (e.g., the first portion) of the intra-oral housing 2180 is configured to snap onto, or otherwise snugly fit, at least a portion of the patient's teeth when the intra-oral housing 2180 is disposed in the patient's mouth for the treatment session. For example, at least a portion of the intra-oral housing 2180 can be biased in a manner similar to that described herein with reference to FIGS. 46 and/or 47. In one or more embodiments, the intra-oral housing 2180 can include one or more retractors configured to facilitate opening of the patient's mouth. In one or more embodiments, at least a portion of the intra-oral housing 2180 can be configured to contact at least a portion of the alveolar soft tissue when the intra-oral housing 1280 is disposed within the patient's mouth for the treatment session. In one or more embodiments, at least a portion of the intra-oral housing 2180 is configured to not contact, but be at a particular distance (e.g., from 0.1 cm to 3 cm) from the alveolar soft tissue when the intra-oral housing 2180 is disposed within the patient's mouth for the treatment session.

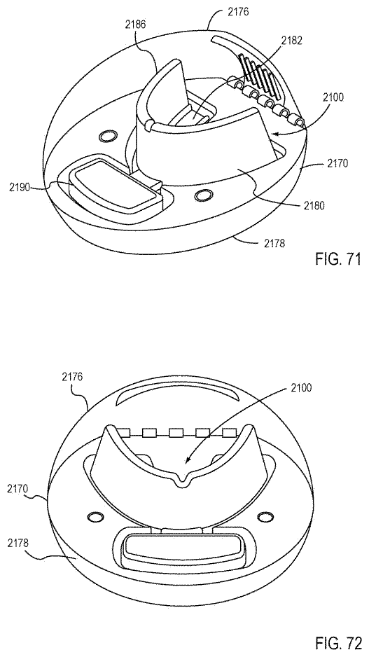

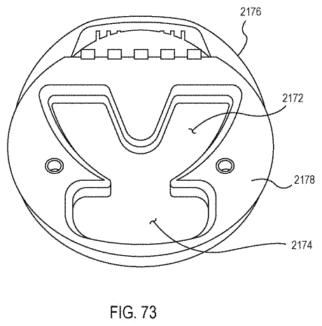

Referring to FIGS. 71-73, the extra-oral housing 2190 can be configured to be disposed on or otherwise coupled to an external station 2170, for example, when the apparatus 2100 is not in use by the patient. The external station 2170 can be, for example a carrying case, charging caddy or station, or the like, or a combination of the foregoing.

The station 2170 includes a base 2178 and a lid 2176 and defines a cavity formed by and between the base 2178 and the lid 2176 when the lid is in a closed position (as shown in FIGS. 71-73). The lid 2176 can be coupled to the base 2178 using any suitable coupling mechanism, for example, using a hinge as shown in FIGS. 71-73. In this manner, the lid 2176 is conveniently moveable between its closed position and an open position (not shown). The base 2178 can define a first recess 2172 configured to receive at least a portion of the intra-oral housing 2180 and a second recess 2174 configured to receive at least a portion of the extra-oral housing 2190. A perimeter of the first recess 2172 can, for example, complement the contour of the intra-oral housing 2180. A perimeter of the second recess 2174 can, for example, complement the contour of the extra-oral housing 2190.

The external station 2170 can be configured to charge the apparatus 2100 when the apparatus 2100 is disposed on or otherwise coupled to the station. In this manner, the battery 2194 can be recharged when the extra-oral housing 2190 is coupled to the charging station. In one or more embodiments, for example, the station 2170 is configured to inductively charge the apparatus 2100. In one or more embodiments, the second end 2193 of the extra-oral housing 2190 includes a connector (not shown in FIGS. 67-74) configured to be coupled to a complementary or mating connector (not shown in FIGS. 67-74) of the external station 2170.

The intra-oral apparatus 2100 can be configured to determine when the intra-oral housing 2180 is disposed within the patient's mouth (i.e., in a manner suitable for the treatment session). In one or more embodiments, for example, the intra-oral apparatus 2100 includes a sensor (not shown in FIGS. 67-74) configured to detect reflection of light off of a patient's oral soft tissue. The sensor can be, for example, a proximity detector included, or embedded, in the flexible circuit 2130. Such a proximity detector can, for example, include any suitable capacitance detection device. The light emitting panel 2102 can be configured to blink or pulse the LEDs included therein, for example, upon removal of the apparatus 2100 from the external station 2170 based, for example, on feedback from the proximity detector. The LEDs can be configured to blink or pulse at a predetermined rate.

At least a portion of light emitted from the pulsing or blinking LEDs towards the oral soft tissue of the patient's mouth is reflected to the intra-oral housing 2180 and is thereby detected by a sensor or other light detection mechanism (generally referred to as a “light sensor” or “photodetector,” not shown in FIGS. 67-74). The light sensor is configured to evaluate the functionality of a portion of the light emitting array 2102 coupled to the left side of the intra-oral housing 2180 and a portion of the light emitting array 2102 coupled to the right side of the intra-oral housing 2180. In this manner, the light sensor facilitates detection of any faulty operation of the apparatus 2100 with respect to each of the left and right sides of the intra-oral housing 2180 before operation of the apparatus 2100 with respect to the patient. Suitable thresholds pertaining to the amount of light detected can be established, and are useful for example to assess whether the LEDs of the light emitting array 2102 are operating properly. The apparatus 2100 can be configured to initiate irradiation of the oral tissue (i.e., begin a treatment session) when the light sensor detects the light reflected from the oral soft tissue. In one or more embodiments, the light sensor is configured to transmit a signal to the microprocessor 2196 to initiate the treatment session when the light sensor detects light reflection (e.g., at or above a predetermined threshold) from the oral soft tissue.

The intra-oral apparatus 2100 can be configured for use in an orthodontic treatment, including any treatment described herein. In one or more embodiments, for example, the intra-oral apparatus 2100 is useful to irradiate at least a portion of the patient's upper jaw for about 3 minutes, the patient's lower jaw for about 3 minutes, or each of the patient's upper and lower jaws for about 3 minutes. More specifically, in one treatment program, the intra-oral apparatus 2100 is useful to administer a light-therapy treatment session in which the oral tissue associated with each of the upper arch of the patient's mouth and the lower arch of the patient's mouth (or vice versa) are consecutively irradiated for 3 minutes per day, for a total treatment session of 6 minutes per day.

During the treatment session, for example, the apparatus 2100 is configured to administer the light therapy using 12 Joules/cm². In one or more embodiments, the 12 Joules/cm² is administered at an intensity of 150 mW/cm² for the three minutes duration. As such, the LEDs tend to remain under a thermal threshold of about 41 degrees Celsius in contact with, or within the particular distance of, oral tissue (and thus under a maximum limit of 43 degrees Celsius). In one or more embodiments, the 12 Joules/cm² can be administered at a higher intensity, such as at an intensity of about 600 mW/cm² for about 20 seconds or about 1 W/cm² for about 12 seconds. In other embodiments, the light is administered at an intensity of about 60-12 mW/cm².

The light is emitted at a wavelength of 850 nm during the treatment session. In one or more embodiments, the light is emitted at a wavelength of 850 nm (±15 nm) during the treatment session. In other words, LEDs can emit light at a blend of wavelengths, and not at a single wavelength like a laser. The peak light emission wavelength (λmax) by the LEDs can be, for example, 855 nm. The treatment sessions can be administered for any suitable period, including, but not limited to, a period of four to twelve months. Such a treatment program can, for example, reduce the duration of an average period a patient is expected to need to use an orthodontic appliance (e.g., braces) to achieve a desired orthodontic result from two years to six months. The foregoing treatment program and/or any treatment program described herein can reduce a duration of an orthodontic treatment administered without light therapy, as described herein, by about 50 percent to about 75 percent.

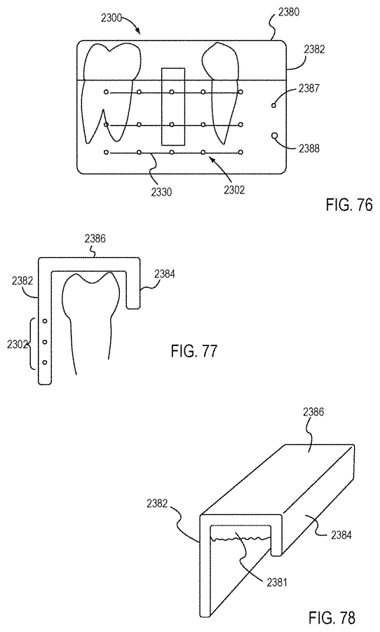

Although the intra-oral housing (e.g., intra-oral housing 780, 880, 980, 1280, 1680, 1780, 1880, 1980, 2080, 2180) have been illustrated and described herein as having an arch shape similar to at least one of the upper or lower arch of a patient's teeth, in other embodiments, an light therapy apparatus can include an intra-oral housing having another suitable configuration. For example, referring to FIG. 75, an intra-oral apparatus 2200 configured to administer light therapy to a patient's oral tissue (e.g., the oral mucosa and/or root area) includes an intra-oral housing 2280 and an extra-oral housing 2290 coupled to the intra-oral housing. The extra-oral housing can be similar in many respects, or identical, to the extra-oral housing 2190 described herein with reference to FIGS. 67-69, and is therefore not described in detail herein. Although the intra-oral housing 2280 is shown and described as being coupled to the extra-oral housing 2290, in other embodiments, the intra-oral housing 2280 can be configured to be electrically coupled to a separate electronic device (e.g., via fiber optic cable(s), or other electronic tether as shown and described with respect to FIG. 66) configured to perform the functions of the components of the extra-oral housing 2290.

The intra-oral housing 2280 includes a light emitting array 2202. The light emitting array 2202 can be the same as or similar in many respects to a light emitting array described herein, and thus is not described in detail with respect to FIG. 65. The intra-oral housing 2280 can be configured to be received in the oral cavity such that a light emitting array 2202 is wholly disposed on the lingual side of the upper and/or lower arches of the patient's teeth. The intra-oral housing 2280 can define a substantially circular perimeter, e.g., having a curvature approximating the curvature of a circle). For example, the intra-oral housing 2280 can be spherical, disc shaped, or the like. For descriptive purposes, the intra-oral housing 2280 can have a shape and can be disposed within a patient's mouth similar to a lollipop. In this manner, the light emitting array 2202 can be disposed adjacent the perimeter of the intra-oral housing 2280 such that the light emitting array 2202 emits light towards the patient's oral tissue (e.g., the oral mucosa and/or the root area) in a direction radiating from a central axis A, as shown by the arrows A1, A2, A3, A4, A5 and A6, in FIG. 75. In one or more embodiments, at least a portion of the intra-oral housing 2280 can be configured to contact at least a portion of the alveolar soft tissue when the intra-oral housing 2280 is disposed within the patient's mouth. In one or more embodiments, at least a portion of the intra-oral housing 2280 is configured to not contact, but be at a particular distance (e.g., from 0.1 cm to 3 cm) from, the alveolar soft tissue when the intra-oral housing 2280 is disposed within the patient's mouth.

Although the intra-oral apparatus have been illustrated and described herein as being configured to administer light therapy to the upper and/or lower arch of a patient's teeth, in one or more embodiments, an intra-oral apparatus is configured to administer light therapy to a portion or section of the patient's oral mucosa (e.g., the alveolar mucosa). For example, referring to FIG. 76, in one or more embodiments, an apparatus 2300 is configured to administer light therapy to three or four teeth of the patient, to a quadrant of the patient's teeth, or to one arch of the patient's teeth. Such an intra-oral apparatus can be beneficial in the case of implantology and/or oral surgery.

The apparatus 2300 includes an intra-oral housing 2380 configured to be disposed within the oral cavity of the patient. The intra-oral housing 2380 defines a first segment 2382, a second segment 2384 and a third segment 2386 coupling the first segment and the second segment. When the intra-oral housing 2380 is disposed within the patient's oral cavity for a treatment session, the first segment 2382 of the intra-oral housing is configured to be disposed (e.g., vertically) between the patient's teeth and the patient's buccal mucosa, the second segment 2384 is configured to be disposed (e.g., vertically) on the lingual side of the crown of the patient's teeth, and the third segment 2386 is configured to be disposed (e.g., horizontally) adjacent and/or on the occlusal surface of the patient's teeth. The second segment 2384 has a sufficient height (i.e., measured in a direction from the occlusal surface to the root area) to inhibit tipping of the intra-oral housing 2380 towards the patient's cheek.

In one or more embodiments, a layer 2381 of moldable material is disposed on an occlusal-facing surface of the third segment 2386 of the intra-oral housing 2380. A moldable impression of the designated teeth can be made using the layer 2381, thus facilitating placement of the intra-oral housing 2380 when the housing is later re-inserted into the oral cavity by the patient (e.g., for a subsequent treatment session).

A flexible circuit 2330 is disposed within the first segment 2382 of the intra-oral housing 2380. The flexible circuit 2330 includes a light emitting array 2302 configured to administer light therapy, in any manner described herein, to the patient's teeth. For example, the flexible circuit 2330 can include a light emitting array 2302 including 15 LEDs, or 15 LEDs per tooth that will be subjected to light therapy. The light emitting array 2302 can includes LEDs configured to administer, or emit, light ranging from about 600 to about 1200 nm. The flexible circuit 2330 of the intra-oral housing 2380 includes a sensor 2387. The sensor 2387 can be the same as or similar in many respect to the sensor shown and described herein with reference to FIG. 25A. The sensor 2387 can be configured to detect the temperature of the apparatus 2300, the patient's alveolar soft tissue and/or the patient's root area. For example, a thermistor or similar temperature measuring device can be placed in the circuit 2330 to monitor the temperature of the light emitting array 2302, as well as measure the temperature inside the patient's mouth. This information can serve as a method for obtaining temperature-related information as well as monitoring patient compliance. When the intra-oral housing 2380, and thus the circuit 2330, is placed in the mouth and when the apparatus 2300 emits light, the temperature of the light emitting array 2302 will rise from pre-treatment ambient temperature to closer to normal body temperature. By monitoring the change in temperature, a controller 2314, described in more detail herein, can monitor the period of time that the light emitting array 2302 is in the oral cavity, based on the period of time the temperature is elevated and close to body temperature.

In one or more embodiments, the circuit 2330 includes a sensor (or proximity detector) that is configured to detect contact of, or a proximity at a particular distance (e.g., from 0.1 cm to 3 cm) from, the first segment and/or light emitted by the light emitting array 2302 with the patient's oral mucosa and/or root area. In this manner, the controller 2314 can detect that the intra-oral housing 2380 is disposed within the patient's oral cavity, and therefore can determine that a treatment session can be initiated.

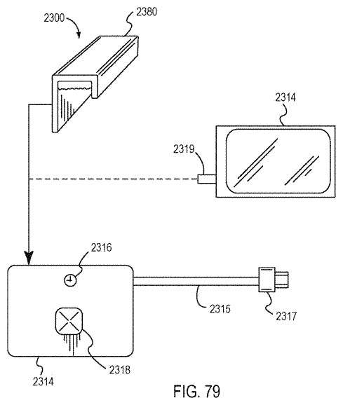

Referring to FIG. 79, the intra-oral housing 2380 is configured to be coupled to an external electronic device, e.g., via a wired or wireless connection. The external electronic device can be configured to provide or convey power to the intra-oral housing 2380, for example, for operation of the light emitting array 2302 during a treatment session. The external electronic device can also be configured to control operation of the light-emitting array 2302.

In one or more embodiments, the external electronic device is a controller 2314. The intra-oral housing 2380 can be removably couplable to the controller 2314. In one or more embodiments, the intra-oral housing 2380 is disposable and the controller 2314 is reusable. In this manner, the intra-oral housing 2380 can be disposed of after a predetermined number of uses and/or after a predetermined period of time, and a second intra-oral housing (not shown) can optionally be used with the controller 2314. The controller 2314 can be electrically coupled to a charger 2317, such as a medical grade USB charger, via a cable 2315, such as a USB cable.

The controller 2314 can be similar in many respects or identical to any controller (e.g., controller 430, 1114) described herein. The controller 2314 can also include many components the same as or similar to those disposed within the extra-oral housing 2190 of apparatus 2100. For example, the controller 2314 can include a microprocessor. Because the microprocessor can be the same as or similar in many respects to any microprocessor described herein (e.g., microprocessor 2196), it is not described in detail herein. The controller 2314 can be preconfigured with a treatment protocol. The controller 2314 includes a button 2318 for initiating a treatment session. The button 2318 can also be configured to, for example, pause or stop a treatment session.

The controller 2314 includes an LED indicator 2316 that can be configured to provide an indicia to the patient of the status of the controller, the intra-oral housing 2380, and/or the treatment program. The LED indicator 2316 can be similar or identical, for example, to the communication mechanism of extra-oral housing 2190, described herein, in that it is configured to indicate a status, or stage, of the treatment session. In one or more embodiments, the LED indicator 2316 is configured to display no light during a first stage of the treatment session, a blinking or pulsed light during a second stage of the treatment session, and/or a solid light during a third stage of the treatment session. The LED indicator 2316 can be, for example, configured to display a solid light for a first predetermined duration (e.g., 2 minutes and 30 seconds, 2 minutes and 45 seconds, or 2 minutes and 10 seconds) upon initiation of the treatment session. The LED indicator 2316 can be configured to display the blinking or pulsed light for a second predetermined duration (e.g., 10, 15 or 30 seconds) following the first predetermined duration as a signal to the patient that the treatment session is nearing its end. The LED indicator 2316 can be configured to display no light when a treatment session is ended (e.g., after 3 minutes from initiation of the treatment session) and the apparatus 2100 is not irradiating light to the patient.

In one or more embodiments, the external electronic device 2314 is a personal electronic device such as a mobile phone (e.g., a smartphone, such as an iPhone®, a tablet, such as an iPad®), a personal digital assistant, or the like. The intra-oral housing 2380 can be coupled to the device 2314 using a connector 2319. In an embodiment in which the device 2390 includes, for example, a smartphone, the smartphone can be configured to perform any operation or function that the controller is configured to perform. For example, the device 2314 can be configured to provide power to the intra-oral housing 2380. In another example, the device 2314 can include an application configured to provide an interface for the patient, control the light-emitting array 2302, and/or record usage information (e.g., compliance information) for subsequent accessing of the information by the patient and/or a physician.

In one or more embodiments, a system includes a first portion configured to administer light therapy to a patient, as described herein, for a first time period, and a second portion configured to administer light therapy to the patient, as described herein, for a second time period different than the first time period. For example, in one or more embodiments, the system includes a plurality of apparatus (or intra-oral housings), such that at least a portion of each apparatus is configured to be disposed within the patient's mouth. Each apparatus of the plurality can include any apparatus or intra-oral housing described herein. For example, the system includes a first intra-oral apparatus and a second intra-oral apparatus distinct from the first intra-oral apparatus. The first apparatus is configured to begin administering light therapy to a patient at a first time period beginning at TO. TO represents the start of a phototherapy session (e.g., corresponding to a date the patient is assigned the first apparatus and/or starts daily usage of the first apparatus). For at least some patients, TO can also represent the day of maxillary bonding and/or the start of an orthodontic treatment. The start of orthodontic treatment can begin, for example, on the day fixed orthodontic brackets and wires are installed on the patient's teeth. The first apparatus can be selected based on a position or configuration of the patient's teeth prior to administration of the light therapy. The first apparatus is configured to administer light at a first wavelength, such as, but not limited to, about 850 nm.

The second apparatus is configured to administer light therapy to a patient at a second time period beginning at T>0, subsequent to T0. In one or more embodiments, the second apparatus is optimally configured to administer light therapy to the patient based on a position of the patient's teeth after administration of at least a portion of the light therapy. For example, the second apparatus can include a light emitting array differently configured from a light emitting array of the first apparatus. The second apparatus can be configured to administer light at a second wavelength different than the first wavelength, such as, but not limited to, about 620 nm. In this manner, the second apparatus can be selected based, at least in part, on tooth movement that occurred during the light therapy administered in combination with the first apparatus and during a time period between T0 and T>0. For example, the first apparatus can be used by the patient at start of the light therapy program and for the first time period, and the second apparatus can be used by the patient beginning about three months after the beginning of the light therapy program and for the second time period. The system can include any suitable number of apparatus, such as two, three, four or more apparatus configured to administer the light therapy. For example, the system can include the first apparatus configured to administer the light therapy beginning at TO, the second apparatus configured to administer light therapy beginning at T1, and a third apparatus configured to administer light therapy beginning at T2.

In another example, the system can include an apparatus having a first light emitting array (e.g., the first portion) and a second light emitting array (e.g., the second portion). The first light emitting array can be configured to administer light at a first wavelength, such as, but not limited to, about 850 nm. The second light emitting array can be configured to administer light at a second wavelength different than the first wavelength, such as, but not limited to, about 620 nm. The first light emitting array and the second light emitting array can be included in a single intra-oral housing. The system, which includes a first portion configured to emit light at the first wavelength for the first time period and at the second wavelength for the second time period, is beneficial at least because it permits a transition during a light therapy program from a higher light wavelength to a lower light wavelength that can help start to increase bone mineralization in the patient's treated area. Such an increase in bone mineralization can facilitate ensuring a more stable result of the moved teeth following an orthodontic treatment.

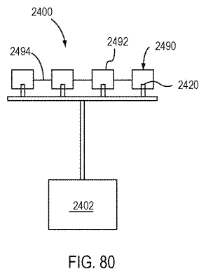

In one or more embodiments, the light therapy apparatus described herein are configured for use in combination with a functional dental appliance, as described in more detail herein. In other embodiments, a light therapy apparatus is integrally formed with a functional appliance configured to exert a force on the teeth of a patient, such as a functional appliance described in more detail herein. For example, referring to FIG. 80, a system 2400 according to an embodiment is configured to regulate tooth movement. The system 2400 includes one or more light emitters (e.g., fiber optical cable(s)) 2420 and an orthodontic bracket system 2490. The light emitters 2420 can be the same as or similar to any light emitter described herein, including, but not limited to, optical fiber cables 420 depicted in FIG. 29. The light emitters 2420 are coupled to the bracket system 2490. For example, the light emitters 2420 can be coupled to one or more brackets 2492 of the bracket system 2490, to one or more wires 2494 of the bracket system 2490, or any combination of the foregoing. In this manner, a separate intra-oral housing is not needed to maintain a position of the light emitters 2420 with respect to the patient's tooth, root area and/or oral mucosa. The light emitters 2420 are couplable to a light source 2402. The light source 2402 can be any suitable light source, including any light source described herein (such as light source 402 depicted in FIG. 29).

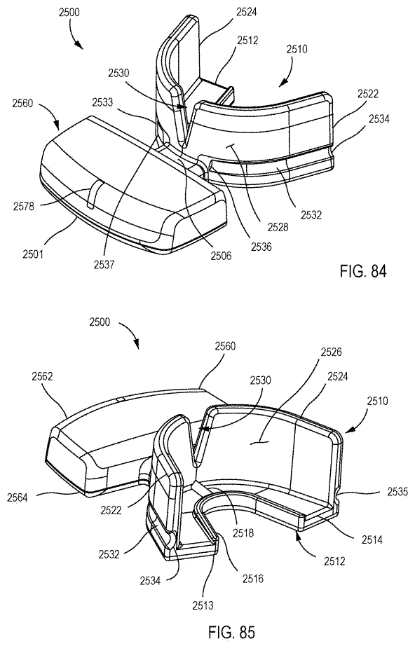

A light therapy system according to an embodiment is illustrated in FIGS. 84-113. The system includes a light therapy apparatus 2500 (see, e.g., FIGS. 84-97) and an external station 2580 (see, e.g., FIGS. 109-113). The light therapy apparatus 2500 is configured to irradiate light in any suitable manner described herein, including, for example, to irradiate the alveolar mucosa and/or root area of the patient. Similarly stated, the light therapy apparatus 2500 is configured to administer light therapy to a patient's teeth and/or oral mucosa. More specifically, the light therapy apparatus 2500 is configured to administer light to the patient's teeth and/or oral mucosa sufficient to accelerate orthodontic movement of the patient's teeth and to reduce the overall treatment time for the patient when undergoing orthodontic treatment. In one or more embodiments, the light therapy apparatus 2500 is configured to administer light to the patient's teeth and/or oral mucosa in an amount sufficient to increase alveolar bone volume and/or bone density. The light therapy apparatus 2500 is useful in combination with traditional orthodontic treatment with an orthodontic appliance, such as brackets, wires, and/or aligners. The light therapy apparatus 2500 can be the same as or similar in many respects to, or include components the same as or similar in many respects to, the intra-oral apparatuses described herein, including, for example, apparatus 2100. The light therapy apparatus 2500 is configured to be disposed in or on the external station 2580 when the apparatus is not in use for a light therapy treatment session, as described in more detail herein.

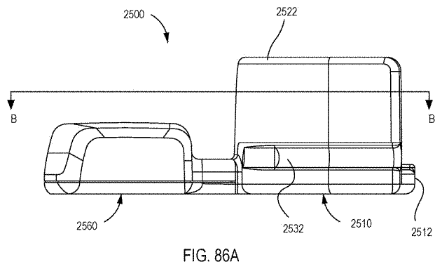

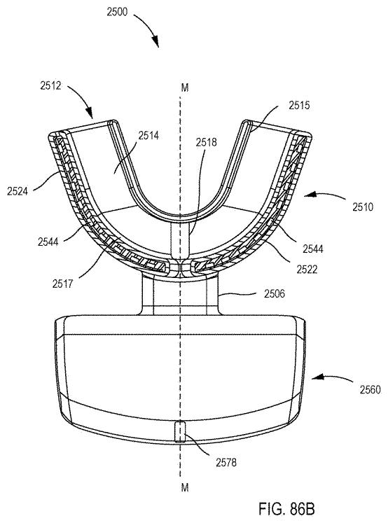

The light therapy apparatus 2500 includes an intra-oral housing 2510 (also referred to herein as a “mouthpiece”) configured to be disposed in an oral cavity (e.g., in the mouth, not shown in FIGS. 84 and 85) of a patient and an extra-oral housing 2560 (also referred to herein as a “bill”) configured to be coupled to the mouthpiece 2510 and disposed externally to the patient's mouth when the mouthpiece 2510 is disposed within the patient's mouth.

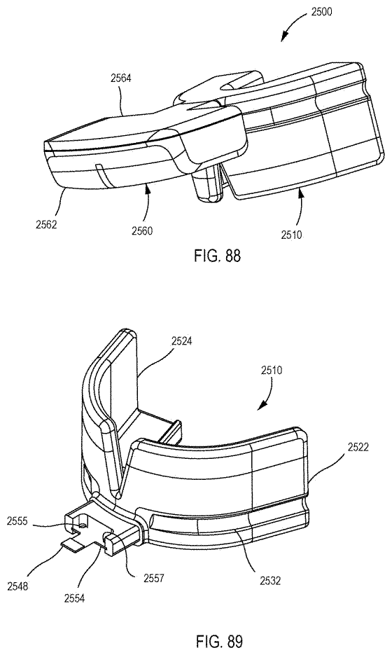

The light therapy apparatus 2500 is configured to be useful for light therapy with the upper jaw and/or the lower jaw of the patient. In other words, the light therapy apparatus 2500 can be configured to administer light therapy with respect to the patient's upper jaw when the apparatus is in an upright position (e.g., as shown in FIG. 84), and can be configured to administer light therapy with respect to the patient's lower jaw when the apparatus is in an inverted position (e.g., as shown in FIG. 88). As such, the mouthpiece 2510 is configured to be disposed within the patient's oral cavity with respect to each of the upper and lower jaws of the patient. Similarly stated, the mouthpiece 2510 is configured matingly adapt to both the upper jaw and the lower jaw, as described herein, thus eliminating the need for a separate mouthpiece for each jaw. It should be noted that although the light therapy apparatus 2500 generally, and the mouthpiece 2510 specifically, can be described as being in the upright position when configured to be oriented with respect to the upper jaw and in the inverted position when configured to be oriented with respect to the lower jaw, in other embodiments, the light therapy apparatus 2500 and the mouthpiece 2510 are in the upright position when configured to be oriented with respect to the lower jaw of the patient, and in the inverted position when configured to be oriented with respect to the upper jaw of the patient.

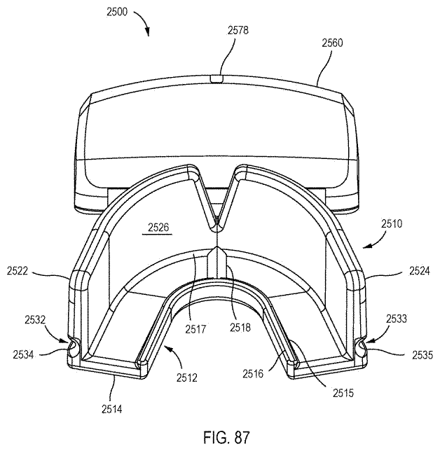

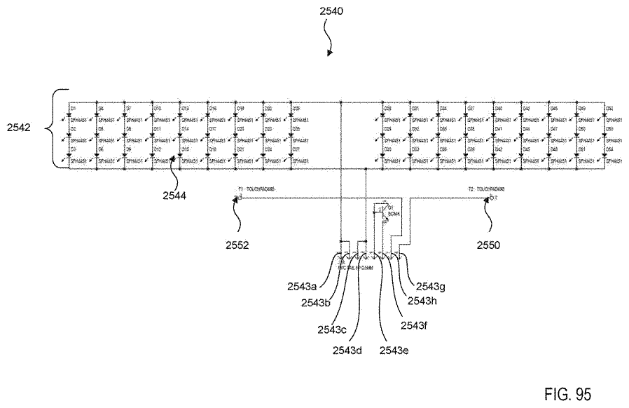

The mouthpiece 2510 can be similar in one or more respects, and include components similar in one or more respects, or identical, to the intra-oral housings described herein, including, for example, the intra-oral housings or mouthpieces described herein with reference to FIGS. 35-37, 65-66 and 67-72. The mouthpiece 2510 includes a bite tray 2512, flanges 2522, 2524, a light array 2542 (see, e.g., FIG. 94), and a support plate 2554 (see, e.g., FIG. 94). The bite tray 2512 is configured to receive at least a portion of the patient's teeth of the upper and/or lower jaw. As such, the bite tray 2512 is generally U-shaped, as shown in FIG. 86B. The bite tray 2512 is configured to facilitate proper positioning of the mouthpiece 2510 within the patient's mouth. The bite tray 2512 generally includes the lower portion of the mouthpiece 2510. The bite tray 2512 includes a bite pad 2514 with an inner perimeter (or side wall) 2515 and an outer perimeter (or side wall) 2517. Flanges 2522, 2524, described in more detail herein, generally define an upper portion of the mouthpiece 2510 and are coupled to the outer perimeter 2517 of the bite pad 2514.

Although one or more mouthpieces (e.g., mouthpiece 2510) are described herein as comprising two flanges (e.g., flanges 2522, 2524) generally disposed on left and right sides with respect to a midline of the mouthpiece (and optionally with respect to a notch generally disposed at the midline), for example flanges that are each disposed proximate an anterior or outer side of the bite tray (e.g., bite tray 2512), the flange in such embodiments can alternatively be referred to as a single flange (e.g., with first and second, or left and right, side portions, and optionally with respect to a notch generally disposed at the midline).

An inner ridge 2516 is coupled to or otherwise formed on the inner perimeter 2515 of the bite pad 2514. The flanges 2522, 2524 of the mouthpiece 2510 and the inner ridge 2516 each extend and/or protrude from the bite pad 2514 in a first direction. As such, when the mouthpiece 2510 is disposed within the patient's mouth, the bite tray 2512 is positioned within the mouth such that the bite pad 2514 is adjacent the occlusal surface of one or more teeth, the flanges 2522, 2524 are disposed between the one or more teeth and buccal tissue, and the inner ridge 2516 is disposed between the one or more teeth and the tongue. More particularly, at least one side of the flange (e.g., a portion including the emitters, as described herein) can contact alveolar mucosa and an opposite side of the flange can contact labial mucosa, when the mouthpiece 2510 is disposed within the patient's mouth. Similarly stated, the bite tray is configured such that when the mouthpiece 2510 is disposed within a mouth, a least a portion of one or more teeth are positioned between the flanges 2522, 2524 and the inner ridge 2516.

The bite tray 2512 can have any thickness suitable for receiving a bite force thereon. In one or more embodiments, the bite pad 2514 can have a constant thickness. In other embodiments, the thickness of the bite pad 2514 can vary spatially. For example, the bite tray 2512, and more specifically, the bite pad 2514, can have a first thickness at an anterior end portion of the bite tray, and a second thickness greater than the first thickness at a posterior end portion of the bite tray. Similarly stated, in one or more embodiments, the thickness of the bite pad 2514 increases along the length of the bite pad 2514 between a first (anterior) portion of the bite pad 2514 and a second (posterior) portion of the bite pad. For example, in one or more embodiments, a thickness of the bite pad 2514 at an anterior portion is between about 5 mm and about 25 mm, and a thickness of the bite pad 2514 at a posterior portion is between about 7 mm and about 27 mm. The increased thickness of the first portion of the bite pad 2514 forces most of the contact between the patient's teeth and the mouthpiece 2510 to be between the posterior teeth (e.g., the molars) and the thicker, second portion of the bite pad 2514. Similarly stated, any pressure exerted by the teeth on the bite tray 2512 will be more concentrated on the thicker portion of the bite pad 2514. Having the greater bite force at the posterior portion of the bite tray 2512 improves patient comfort and helps to avoid damage to the mouthpiece 2510 that might otherwise be caused by the patient's sharper anterior teeth.

Additionally, the increased contact interface between the bite tray 2512 and the posterior teeth also provides for a more universal patient fit, because positional variability of the posterior teeth is often less than that of the anterior teeth. Similarly stated, this arrangement produces a more repeatable (treatment-to-treatment and patient-to-patient) fit because the position of a patient's posterior teeth often varies less amongst different patients' anatomies than the position of the anterior teeth and incisors. In one or more embodiments, the bite pad 2514 is constructed (i.e., is constructed from a material and/or has a sufficient thickness) to withstand a biting force of up to about 340 N. In one or more embodiments, the bite pad 2514 has shear and/or fatigue strength to withstand 50 N of force repeatedly applied to the bite tray 2514.

As shown in FIG. 87, an upper surface of the bite pad 2514 includes a ridge 2518. The ridge 2518 is disposed along a midline M of the mouthpiece 2510 and is elevated with respect to the upper surface of the bite tray 2512 and/or bite pad 2514. The ridge 2518 can extend between the inner perimeter 2515 of the bite pad 2514 and the outer perimeter 2517 of the bite pad 2514, as best shown in FIG. 86B. The ridge 2518 facilitates positioning of the mouthpiece 2510 within the patient's oral cavity. For example, the mouthpiece 2510 is configured to be positioned within the patient's oral cavity such that the ridge 2518 is disposed between the patient's front central incisors (on either the upper jaw or the lower jaw). Proprioception of the patient related to the teeth and periodontium can produce sensory feedback to the patient regarding the position of the ridge 2518 of the mouthpiece 2510.

In this manner, the ridge 2518 facilitates centering of the mouthpiece 2510 within the oral cavity, thus promoting symmetry of a light therapy treatment on the alveolar mucosa, or other oral tissue, on both sides of the patient's mouth. In other words, in order to promote the symmetrical administration of light therapy to the root area, the mouthpiece 2510 can be positioned with the midline M of the mouthpiece 2510 seated along the sagittal plane or within (i.e., plus or minus) 5 degrees of the sagittal plane, and the ridge 2518 can facilitate such positioning in use. The ridge 2518 can have any suitable shape, including, for example, the shape of an inverted V as shown in FIG. 87, such that the point of the V can be disposed between the patient's front central incisors.

As described herein, the upper portion of the mouthpiece 2510 includes a first (or left) flange 2522 and a second (or right) flange 2524. The upper portion (i.e., the flanges 2522, 2524) of the mouthpiece 2510 is disposed transversely with respect to the bite plate 2514. The flanges 2522, 2524 are configured to be disposed, when the mouthpiece 2510 is disposed within the patient's mouth such that the bite tray is adjacent an occlusal surface of the patient's teeth, adjacent a portion of a side of the patient's teeth and/or adjacent the alveolar mucosa. In this manner, the light array 2542, enclosed in the flanges 2522, 2524, as described in more detail herein, can be useful for administering light to the patient's teeth and/or alveolar mucosa.

The flanges 2522, 2524 collectively contain the light array 2542, and are each configured to be disposed between the buccal tissue and the alveolar mucosa. Thus, in use, the flanges 2522 and 2524 displace oral soft tissue to maintain the desired position of the light array 2542 relative to the anatomy of the patient. More specifically, the flanges 2522, 2524 are each configured to displace buccal tissue away from the patient's alveolar mucosa. In one or more embodiments, an inner face 2526 of the flanges 2522, 2524 can be spaced apart from the patient's alveolar tissue when the mouthpiece 2510 is disposed within the patient's mouth and the flanges 2522, 2524 are displacing the buccal tissue. In one or more embodiments, at least a portion of the inner face 2526 of the flanges 2522, 2524 can contact the patient's alveolar tissue when the mouthpiece 2510 is disposed within the patient's mouth and the flanges 2522, 2524 are displacing the buccal tissue.

The flanges 2522, 2524 of the mouthpiece 2510 are configured to be flexible and/or deformable. Similarly stated, the flanges 2522, 2524 are constructed from a material and have geometrical dimensions and/or configurations to provide the desired flexibility, as described herein. Moreover, each of the first and second flanges 2522, 2524 are independently deflectable, movable and/or deformable with respect to the mouthpiece 2510 and/or each other. In this manner, the mouthpiece 2510 can be easily disposed within the oral cavity for a variety of different patients having a variety of different anatomical structures, as described herein.

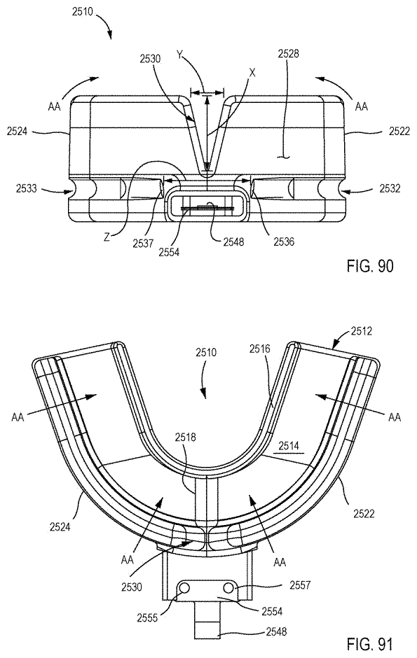

For example, the mouthpiece 2510 includes particular geometric features (e.g., stress concentration risers, areas having a desired bending moment of inertia, etc.) to produce the desired flexibility, deformability and durability in connection with the material(s) from which the mouthpiece 2510 is constructed. As shown, the mouthpiece 2510 defines a notch 2530 and grooves 2532, 2533 configured to permit, or otherwise increase the ability of, the flanges 2522, 2524 to deflect inwardly towards the teeth, gums, jaw, or the like, as shown by the arrows AA in FIG. 90. As shown in FIG. 90, the mouthpiece 2510 defines the notch 2530 at the midline M (see, e.g., FIG. 86B) of the mouthpiece and between upper portions of the first flange 2522 and the second flange 2524. The notch 2530 is configured to permit the independent and/or inward deflection of each of the first flange 2522 and the second flange 2524, for example, in response to pressure from the patient's lip or inner cheek. In particular, the flanges 2522, 2524 are each configured to deflect inwardly with respect to the bite pad 2514. Similarly stated, when the mouthpiece 2510 is outside of the mouth in an undeformed state (i.e., a first configuration), the first flange 2522 and the second flange 2524 are each approximately perpendicular (i.e., at about 90 degrees) to the bite pad 2514. When the mouthpiece 2510 is disposed inside the mouth, the upper portion of the mouthpiece 2510 and/or the flanges 2522, 2524 are sufficiently flexible such that an angle formed between each flange 2522, 2524 and the bite pad 2514 (a “flange angle”) is acute. This “tipping in” allows the flanges 2522, 2524 to conform to the interior surfaces of the mouth, thereby promoting the desired alignment of the light array 2542 relative to the bone and/or teeth. In other embodiments (not shown), the notch 2530 can be absent, and the mouthpiece 2510 can be substantially rigid to prevent deflection of the flanges 2522, 2524.

As shown in FIG. 90, the notch 2530 can be V-shaped, and has a depth X and a width Y at the widest point of the notch. The width Y is at the upper end of the notch adjacent the free end of the flanges 2522, 2524 opposite the bite tray 2512, and is less than the depth X of the notch. The edge of each flange 2522, 2524 that forms a respective side of the notch 2530 tapers towards the point of the V, which point can be aligned with an upper edge of the grooves 2532, 2533. Similarly stated, the portion of the mouthpiece 2510 that defines a lower boundary of the notch 2530 is aligned, in a horizontal plane, with an upper edge of the portion of the mouthpiece 2510 that defines the grooves 2532, 2533. In other embodiments, however, the portion of the mouthpiece 2510 that defines a lower boundary of the notch 2530 can be in any suitable location relative to the grooves 2532, 2533 (e.g., either above or below the grooves).

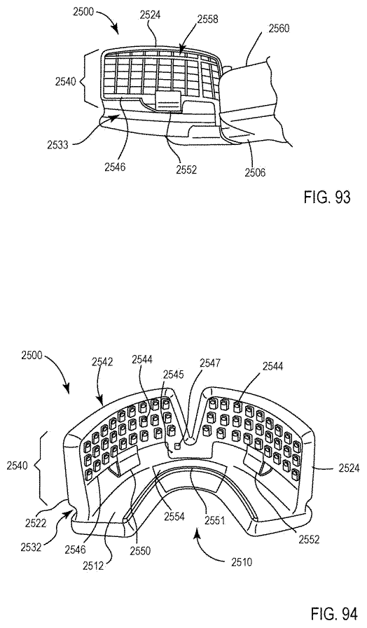

The mouthpiece 2510 defines at least one groove 2533, 2534 defined by a lower outer (or front) surface of each of the first and second flanges 2522, 2524. For example, as shown in FIG. 90, the mouthpiece 2510 includes a first groove 2532 and a second groove 2533, each defined by the outer or front surface 2528 of the mouthpiece 2510. In particular, each groove is disposed at a height between the bite pad 2514 and a lower edge of the flexible circuit board 2546 (see, e.g., FIG. 93). Stated another way, the grooves 2532, 2533 can be defined by a base portion of each of the first and second flanges 2522, 2524. The grooves 2532, 2533 each extend about the outer surface 2528 of the mouthpiece 2510 between the posterior end portion of the mouthpiece 2510 and an anterior end portion of the mouthpiece 2510, such that a first end 2534, 2535 of each groove 2532, 2533, respectively, is at or proximate to the posterior end portion of the mouthpiece 2510 and a second end 2536, 2537 of each groove 2532, 2533, respectively, is at or proximate to the anterior end of the mouthpiece 2510.

As shown in FIG. 90, the second ends 2536, 2537 of the grooves 2532, 2533 can be spaced apart. In other words, the second ends 2536, 2537 of the grooves 2532, 2533 do not necessarily meet at the anterior end of the mouthpiece 2510. Similarly stated, the grooves 2532, 2533 are noncontiguous and/or do not share a common boundary. For example, the second ends 2536, 2537 of the grooves 2532, 2533 can be spaced apart by a width of a bridge 2506 extending from the front of the mouthpiece 2510. In another example, as shown in FIG. 90, the second ends 2536, 2537 of the grooves 2532, 2533 can be spaced apart by a distance Z (which can be at least as great as the width Y of the notch 2530). The grooves 2532, 2533 can have any suitable shape, including, for example, that of a semi-circle or U-shape. The grooves 2532, 2533 produce a hinge-like structure (i.e., a “living hinge”) about which the flanges 2522, 2524 can rotate, bend and/or deflect. In this manner, the grooves 2532, 2533 and the notch 2530 collectively permit the flanges 2522, 2524 to deflect inwardly, for example, in response to pressure from the patient's lip or inner cheek.

As such, the grooves 2532, 2533 and the notch 2530 collectively facilitate the transition of the mouthpiece 2510 between a first configuration and a second configuration. When the mouthpiece 2510 is in the first configuration, the angle formed between each flange 2522, 2524 and the bite pad 2514 (the “flange angle”) has a first value. When the mouthpiece 2510 is in the second configuration, the flange angle has a second value that is different from the first value. In particular, the mouthpiece 2510 can be moved to the second configuration when disposed within the patient's mouth. In one or more embodiments, the second value is less than the first value (i.e., the flanges 2522, 2524 “tip” inward when the mouthpiece 2510 is inserted into the mouth). In one or more embodiments, the flange angle is approximately 90 degrees when the mouthpiece is in the first configuration and is acute when the mouthpiece is in the second configuration. In one or more embodiments, the flange angle is about 80 degrees (e.g., the flanges 2522, 2524 tip inward by about 10 degrees) when the mouthpiece is in the second configuration. In other embodiments, the flange angle is between about 75 degrees and about 80 degrees (e.g., the flanges 2522, 2524 tip inward by between about 10 degrees and 15 degrees). In yet other embodiments, the flange angle is approximately 85 degrees, 75 degrees, 70 degrees, or 65 degrees (e.g., the flanges 2522, 2524 tip inward by about 5 degrees, 15 degrees, about 20 degrees and about 25 degrees, respectively) when the mouthpiece is in the second configuration.

Without wishing to be bound by theory, it is thought that the flexibility of the mouthpiece 2510, and of the flanges 2522, 2544, provides significant advantages. For example, in contrast to mouthpieces constructed of a hard plastic and/or with a permanent set (or shape), the current arrangement allows for easier insertion and better conformance to the oral tissue of the patient. The flexibility of the mouthpiece 2510 also accommodates variation in patient anatomy (whether between two different patients or for the same patient as that patient's anatomy changes over time). For example, some patients have a pronounced overbite and might need more or less than a 10 degree inward deflection (or “tip-in”). In such instances, the mouthpiece 2510 can conform to the internal structure and/or anatomy within the patient's mouth. As another example, as the orthodontia for a patient works over time, the patient's dental anatomy will change. Accordingly, the mouthpiece 2510 can conform to the internal structure and/or anatomy within the patient's mouth to accommodate such change without requiring new mouthpiece moldings or the like. Finally, the flexible design of the mouthpiece 2510 provides greater comfort for the patient than would be provided by mouthpieces constructed of a hard plastic.

Additionally, the flexible nature of the mouthpiece 2510 and/or the flanges 2522, 2524 can provide manufacturing benefits. In particular, fabrication and/or molding of a mouthpiece having an acute angle between the bite surface and the side surface of the flange (i.e., the internal angle of the flange or the “flange angle”) can be difficult. The design of the mouthpiece 2510, however, allows for the molding and/or fabrication to be performed with a flange angle of approximately ninety degrees (or greater), while allowing for an in-use flange angle that is acute (e.g., when the mouthpiece 2510 is in the second configuration, as described herein).

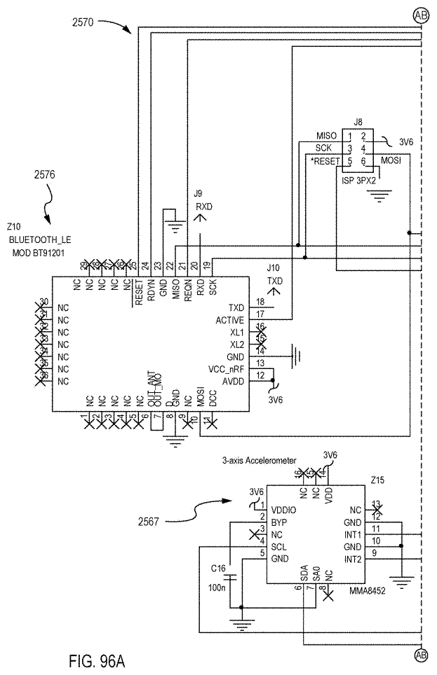

The mouthpiece 2510 of the light therapy apparatus 2500 includes an electronics assembly 2540, generally shown in FIGS. 93 and 94, and schematically in FIG. 95. In one or more embodiments, at least one or more portions of the mouthpiece 2510 are constructed from a substantially transparent material (e.g., silicone) such that one or more components embedded within the mouthpiece 2510 are visible through the mouthpiece 2510. Thus, for purposes of illustration, portions of the mouthpiece 2510, including portions of the first flange 2522, the second flange 2524 and the bite tray 2512 are shown as being transparent in FIGS. 93 and 94 to show portions of the electronics assembly 2540 and the support plate 2554 disposed therein. The electronics assembly 2540 is configured to cooperatively function with the electronics board 2570 disposed within the bill 2560 to produce the light therapy as described herein. As shown, the electronics assembly 2540 of the mouthpiece 2510 is disposed primarily in the first flange 2522 and the second flange 2524. The electronics assembly 2540 comprises a light array 2542, a flexible circuit board 2546, a pair of capacitance sensors 2549, and one or more conductive components for heat transfer, such as the conductive heat transfer tiles 2558. FIG. 95 schematically illustrates at least a portion of the electronics assembly 2540 of the mouthpiece 2510, according to one or more embodiments. The light array 2542 comprises one or more light emitters 2544, such as a plurality of LEDs (only one light emitter 2544 in each flange is identified in FIG. 95). The light emitters 2544 are electrically and/or physically coupled to the flexible circuit board 2546 (see also, FIG. 86B). The flexible circuit board 2546 electrically couples the light emitters 2544 to electronic circuitry in the bill 2560, such as via pathways 2543a, 2543b, 2543c, 2543d (see, e.g., FIG. 95). In this manner, the light emitters 2544 can receive power and/or a signal to produce the desired light, as described herein.

Referring to FIG. 94, the light emitters 2544 are disposed on a first, palatial (or lingual) side of the flexible circuit board 2546. In this manner, the light emitters 2544 are configured to emit light toward a patient's teeth and/or adjacent oral tissue when the mouthpiece 2510 is disposed within the patient's mouth. The light emitters 2544 can be configured to emit light at any suitable intensity, wavelength and/or frequency described herein, and can be arranged in two or more rows or a single row on the flexible circuit board 2546. For example, in one or more embodiments, the light emitters 2544 can be configured to emit light in the infrared or near infrared wavelength range. For example, in one or more embodiments, the light emitters 2544 are configured to emit light at a wavelength of about 850 nm. In one or more embodiments, the light emitters 2544 are configured to emit light at a wavelength of 850 nm±5 nm. The light emitters 2544 can be configured to emit light sufficient deliver light energy to the patient's bone to facilitate and/or perform any of the methods described herein. The light emitters 2544 can be configured to emit light at less than 150 mW/cm².

The light emitters 2544 can be disposed on the flexible circuit board 2546 and/or within the flanges 2522, 2524 in any suitable configuration, including any configuration described herein. As described herein, the flange that comprises the light emitters can comprise a notch. In another embodiment, the flange that comprises the light emitters does not comprise a notch. In one or more embodiments, the light emitters 2544 are LEDs coupled to the flexible circuit board 2546 in two or more parallel rows and/or columns. As schematically shown in FIG. 95, the light array 2542 can comprise about 54 light emitters 2544, or LEDs, with about 27 light emitters embedded in the first flange 2522 and about 27 light emitters 2544 embedded in the second flange 2524. The 27 light emitters 2544 can be arranged in any suitable configuration, including for example in nine evenly spaced columns with three spaced apart LEDs per column. In another embodiment, the light emitters are arranged in individual columns with one LED per column. In a further embodiment, the columns are evenly spaced. The flexible circuit board 2546 and light emitters 2544 can have any suitable dimensions for being coupled to, or embedded in, the flanges 2522, 2524 of the mouthpiece 2510. The light emitters 2544 can be coupled to, or embedded in, the flanges 2522, 2524 of the mouthpiece 2510 such that the light emitters are spaced apart from the bite tray 2512 by a predetermined distance, for example a distance within the range of from about 5 mm to about 35 mm from the bite tray 2512. In some embodiments, the distance is within the range of from about 5 mm to about 25 mm from the bite tray 2512. In this manner, the light emitters 2544 are positioned with respect to the mouthpiece such that the flange contacts alveolar mucosa and the light emitters embedded in the flange overlie alveolar mucosa when the mouthpiece is disposed within the mouth. Also in this manner, the light emitters 2544 can emit light directly to or in other embodiments towards the alveolar mucosa. Light emitted from the light emitters 2544 (e.g., directly) to or in other embodiments towards the alveolar mucosa in the foregoing manner can reach one or more tooth roots. In some embodiments where light is administered directly to the alveolar mucosa, the light reaches the alveolar mucosa without first reflecting from another region. Although the light emitters 2544 are shown as being evenly spaced within the first flange 2522 and the second flange 2524, in other embodiments, the light emitters can be unevenly spaced within the first flange 2522 and/or the second flange 2524. For example, in one or more embodiments, a mouthpiece can comprise a series of light emitters that are spaced apart by a first amount near the anterior portion of the mouthpiece and a second, different amount near the posterior portion of the mouthpiece.

As shown in FIG. 93, the one or more conductive heat transfer tiles 2558 are disposed on a second, or buccal, side of the flexible circuit board 2546 of the mouthpiece 2510. The tiles can be constructed of any suitable conductive material (e.g., copper, aluminum or the like) and are configured to promote heat transfer away from the light therapy apparatus 2500 and/or the light emitters 2544 and to the patient's buccal tissue. Similarly stated, in use, the copper tiles 2558 can transfer heat from the apparatus 2500 to the patient's cheek, where the patient's naturally occurring circulatory system will draw the heat away from the cheek area. In this manner, the tiles 2558 can facilitate the compliance with applicable temperature regulations and industry standards (e.g., IEC 60601, IEC 60601-2-57, IEC 60601-1-11, EN 62471, etc., described herein) during light therapy administration.

The tiles 2558 can be operatively coupled to the light emitters 2544 in any suitable manner. In one or more embodiments, the flexible circuit board 2546 has a layer on its second side that comprises the one or more heat transfer tiles 2558. The tiles 2558 are spaced apart from each other, and thus can be characterized as being discrete. The discrete nature of the tiles 2558 enhances, or at least does not lessen, the flexibility of the flanges 2522, 2524 of the mouthpiece 2510. In other embodiments, however, the tiles 2558 need not be discrete elements.

In one or more embodiments, at least a portion of the flexible circuit board 2546 is disposed within the bridge 2506 of the light therapy apparatus 2500. For example, the flexible circuit board 2546 can comprise a tab portion 2548 disposed in the bridge 2506. The tab portion 2548 of the flexible circuit board 2546 is configured to electrically couple the electronics assembly 2540 of the mouthpiece 2510 with electronic components, described in more detail herein, disposed in the bill 2560. In this manner, the electronic components disposed in the bill 2560 can control operation of the apparatus 2500, and emission of light using the light array 2542, as described in more detail herein.

The mouthpiece 2510 can be constructed of any suitable material, including, for example, an elastomeric material (e.g., a soft silicone). The terms hardness (or softness, as applicable), strength and/or resistance to deformation are used herein to denote a one or more properties associated with the mouthpiece 2510. The properties include, for example, material properties, such as the yield strength, the modulus of elasticity, the modulus of rigidity, the hardness and/or the elongation percentage. The hardness of a material or the mouthpiece 2510 can be characterized as its “durometer,” in reference to the apparatus used to measure the hardness of the types of material used to form mouthpieces.

In one or more embodiments, the mouthpiece 2510 can be fabricated from medical-grade injection-molded, highly flexible and very low durometer silicone. For example, during manufacture of the mouthpiece 2510, the silicone can be overmolded onto at least a portion of the electronics assembly 2540 of the mouthpiece 2510, including one or more of the flexible circuit board 2546, light emitters 2544, and the copper tiles 2558. In this manner, the portion of the electronics assembly 2540, such as one or more of the flexible circuit board 2546, light emitters 2544, and the copper tiles 2558, are fully encapsulated or embedded within the molded silicone. In this manner, the electronics can be protected for repeated applications within the mouth. In one or more embodiments, the silicone can be have a hardness (or softness) of about 22 Shore A. In one or more embodiments, the silicone can be have a hardness (or softness) of about 30 Shore A. Although soft, the silicone is tear resistant, a desirable characteristic because of the sharpness of a patient's teeth (and the anterior teeth in particular). Moreover, the silicone is hydrophobic, and therefore will not absorb water. Suitable silicones include those offered by Bluestar Silicones, East Brunswick, N.J. under the name Silbione®, including Silbione® LSR 4305, Silbione® LSR 4310, Silbione® LSR 4325, and Silbione® LSR 4325 PEX (www.bluestarsilicones.com).

In one or more embodiments, the silicone and/or portions of the mouthpiece 2510 are substantially transparent, such that one or more components embedded within the silicone are visible through the silicone. Moreover, in this manner, the mouthpiece 2510 can provide suitable optical properties for allowing the light produced and/or conveyed by the light emitters 2544 to pass through the mouthpiece 2510 to the desired target tissue. In one or more embodiments, the mouthpiece 2510 and/or the flanges 2522, 2524 can comprise one or more components configured to filter, focus and/or otherwise act upon the light produced by the light emitters 2544. In other embodiments, the mouthpiece 2510 can comprise air gaps between the light emitters 2544 and the surface of the flanges 2522, 2524 to facilitate focusing of the light. As shown in FIG. 86B, however, the mouthpiece 2510 is constructed such that the light emitters 2544 are fully encapsulated or embedded within the molded silicone such that no space or air gap exists between the silicone material and the portion of the electronics assembly 2540. Similarly stated, the mouthpiece 2510 is devoid of an air gap between the light emitters 2544 and the material of the mouthpiece 2510, thus no air gap lensing is needed to produce the desired optical properties of the light produced by the light emitters 2544.

The ridge 2518 can be constructed of the same material as the mouthpiece 2510, or at least the same material as the bite tray 2512 of the mouthpiece 2510. In this manner, when a patient bites together with the upper and lower jaw, the bite tray 2512 of the mouthpiece 2510, including the ridge 2518, can deform slightly from pressure exerted by an occlusal surface of the patient's teeth. Nonetheless, the ridge 2518 is of sufficient dimensions that the patient should be aware of its position, despite any slight deformation of the bite tray 2512 and/or ridge 2518.

The extra-oral housing, or bill, 2560 is coupled to a front portion of the mouthpiece 2510 by the bridge 2506. In this manner, the bill 2560 is disposed exterior to the oral cavity of the patient when the mouthpiece 2510 is disposed within the oral cavity of the patient. Also in this manner, the bill 2560 can be supported with respect to the patient's mouth by the mouthpiece 2510 and/or the bridge 2506 when the mouthpiece 2510 is disposed within the patient's mouth.

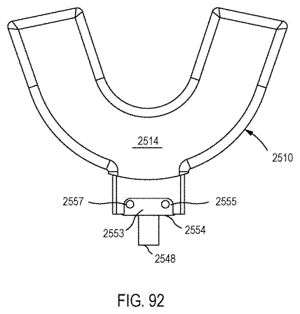

The apparatus 2500 can comprise a support plate 2554. The support plate 2554 is configured to provide structural support to the silicone material of the mouthpiece 2510. The support plate 2554 is configured to help support the bill 2560 with respect to the mouthpiece 2510, for example, when the mouthpiece 2510 is disposed within the patient's mouth. The support plate 2554 has a proximal portion 2551 (see, e.g., FIG. 94) and a distal portion 2553 (see, e.g., FIG. 92). The proximal portion 2551 of the support plate 2554 is coupled to and/or within the mouthpiece 2510. For example, the proximal portion 2551 of the support plate 2554 can be embedded in the silicone material of the bite pad 2514. The support plate 2554 can be substantially planar (e.g., with crests or troughs in the plane having a height no more than 5% of a thickness of the support plate 2554), and the proximal portion 2551 of the support plate 2554 can be disposed within the mouthpiece 2510 such that the support plate is substantially parallel (e.g., plus or minus about 5 degrees) to the upper surface of the bite pad 2514. At least a portion of the support plate 2554 is disposed in the bridge 2506. The distal portion 2553 of the support plate 2554 is configured to couple the mouthpiece 2510 and the bill 2560. For example, the distal portion 2553 of the support plate 2554 can define two apertures 2555, 2557 each configured to receive a protrusion, or guide pin, (not shown in FIGS. 92 and 94) of the bill 2560. In one or more embodiments, at least the distal portion 2553 of the support plate 2554 defining the apertures 2555, 2557 is exposed with respect to (or is not disposed within) the material of the mouthpiece 2510.

The bill 2560 of the light therapy apparatus 2500 comprises a first, or top, portion 2562 and a second, or bottom, portion 2564 and forms a cavity (not shown) therebetween. Although the bill 2560, in combination with the bridge 2506, is shown in FIGS. 84-88 as generally having a T-shape, in other embodiments, the bill 2560 can have any suitable shape. The bill 2560 is configured to at least partially enclose one or more electronic components of the apparatus 2500, as described in more detail herein, which components can be disposed in the cavity of the bill 2560.

The apparatus 2500 comprises at least one battery, or other suitable power source. For example, a battery 2568 is disposed in the cavity of the bill 2560. The battery 2568 can be electrically coupled to and to provide power to one or more electronic components of the bill 2560, including, for example, one or more of an electronics board 2570, a microcontroller 2572, a system clock, a wireless transmitter 2576, and other electronic components of the bill 2560. The battery 2568 is configured to provide power to the electronics assembly 2540 of the mouthpiece 2510. More specifically, the battery 2568 is configured to provide power to the light array 2542 to enable the light emitters 2544 to irradiate light during a treatment session. The battery 2568 can include, for example, a rechargeable lithium ion battery. In one or more embodiments, the battery is a lithium-ion polymer battery, also referred to as a lithium polymer or LIPO battery. In one or more embodiments, the battery 2568 is disposed within the cavity of the bill 2560 between an accelerometer 2567 and the electronics board 2570.

In one or more embodiments, the apparatus 2500 is configured to wirelessly charge, or recharge, the battery 2568. For example, an induction receiver coil 2569 can be disposed in the cavity of the bill 2560. The induction receiver coil 2569 is configured for inductively charging the battery 2568, as described in more detail herein. The induction receiver coil 2569 can comprise, for example, a Qi-based charging coil.

The electronics board 2570 is disposed in the bill 2560 of the apparatus 2500. The electronics board 2570 is electrically coupled to the flexible circuit board 2546 of the mouthpiece 2510 (e.g., via the tab portion 2548 of the flexible circuit board 2546), thereby electrically coupling electronic components of the bill 2560 with the electronic assembly 2540 of the mouthpiece 2510. Electronic circuitry within the bill 2560 electrically couples the electronics board 2570, the microcontroller 2572, the system clock, the wireless transmitter 2576, one or more switches, and other electronic components of the bill 2560.

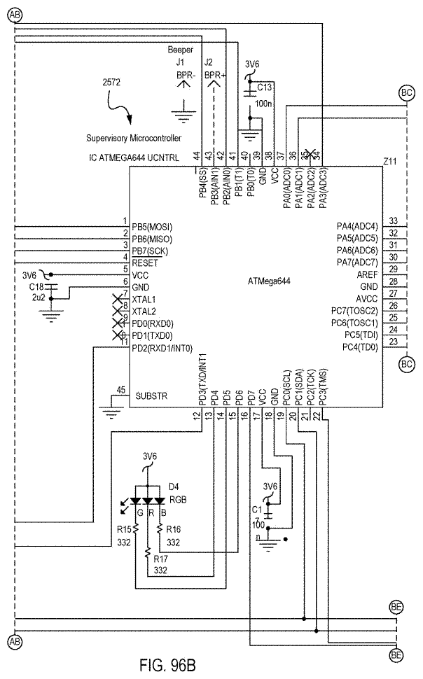

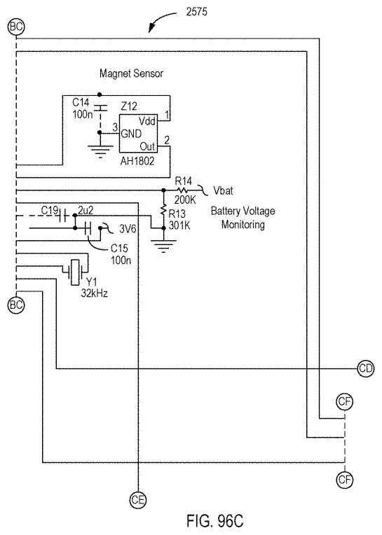

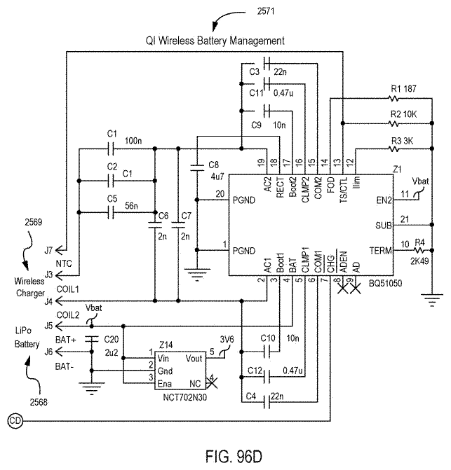

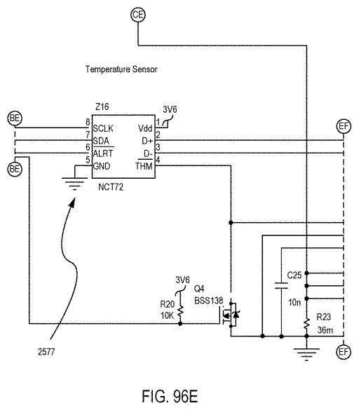

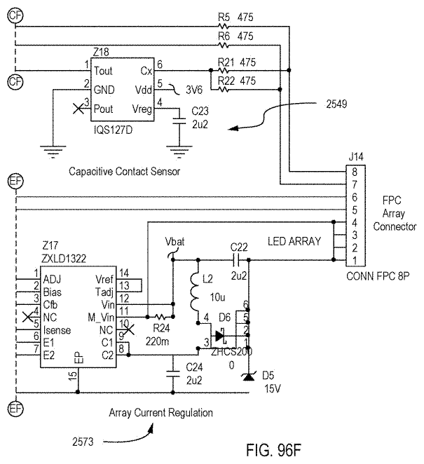

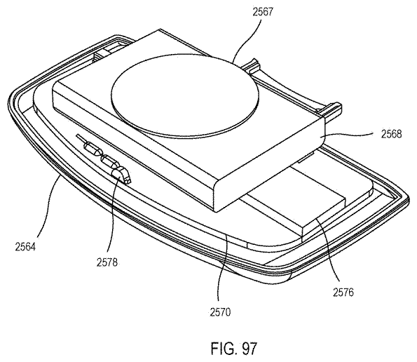

The light therapy apparatus 2500 is configured to detect movement of the apparatus. More specifically, the apparatus 2500 is configured to detect when the apparatus is moved in any one of three axes or dimensions (also referred to herein as three-dimensional movement). Referring to FIGS. 96A-96F, in one or more embodiments, the apparatus 2500 comprises an accelerometer 2567 disposed within the cavity of the bill 2560. The accelerometer 2567 can be, for example, a piezoelectric sensor. The accelerometer 2567 can be coupled to the bill 2560 (e.g., to the first portion 2562 of the bill) by any suitable coupling mechanism, including, but not limited to, an adhesive, such as double-sided tape. The accelerometer 2567 is electrically coupled to the electronics board 2570. In one or more embodiments, two electronic leads or wires (not shown in FIG. 96A-96F or 97) couple the accelerometer 2567 and the electronics board 2570. The accelerometer 2567 is configured to detect three-dimensional, or three-axis, movement of the light therapy apparatus 2500.

The accelerometer 2567 can be configured to send an electrical signal to a microcontroller 2572 of the apparatus 2500 when the three-dimensional movement is detected. The microcontroller 2572 is disposed in the cavity of the bill 2560. The microcontroller 2572 is in electrical communication with the accelerometer 2567, and is configured to receive the electrical signal from the accelerometer 2567. The detected movement of the apparatus 2500 can be useful for controlling the light emissions and/or other aspects of the performance of the apparatus. For example, in one or more embodiments, when the microcontroller 2572 detects movement of the apparatus 2500, the microcontroller 2572 can move the apparatus 2500 from a “sleep” state (in which the light emitters 2544 are prevented from being actuated) to a “wake” state (in which the light emitters 2544 are enabled).

In one or more embodiments, the light therapy apparatus 2500 can be configured to determine the orientation of the apparatus. Stated another way, the light therapy apparatus 2500 can be configured to determine if the mouthpiece 2510 is oriented in the upright or inverted position. For example, in one or more embodiments, the accelerometer 2567 is configured to determine if the apparatus 2500 is oriented in the upright or inverted position. The accelerometer 2567 is configured to send a signal associated with the orientation of the apparatus 2500 to the microcontroller 2572 (see FIG. 96B). In this manner, the electronics board 2570 and/or the microcontroller 2572 can adjust and/or control the operation of the apparatus 2500 as a function of the orientation of the mouthpiece 2510, as described herein.

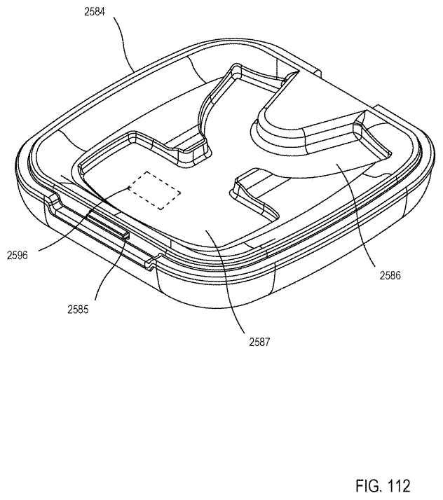

Electronic circuitry disposed in the bill 2560 of the apparatus 2500 comprises a switch 2575 (also referred to herein as a magnet switch or sensor, schematically illustrated in FIG. 96C) that is moveable from a first position (or configuration) to a second position (or configuration). More specifically, the switch 2575 is configured to move from its first position to its second position when the switch is activated by a magnet located in close proximity to the switch. When the switch 2575 is in its second position, the apparatus 2500 is maintained in its sleep state, even if movement is detected by the accelerometer 2567. In one or more embodiments, the external station 2580 comprises the magnet 2596 (see e.g., FIG. 112) configured to activate the switch 2575. In this manner, when the apparatus 2500 is disposed on the external station 2580, the magnet 2596 of the external station 2580 causes the switch to move from its first position to its second position, thereby preventing the apparatus 2500 from moving to its wake state from its sleep state, regardless of whether the accelerometer 2567 detects movement of the apparatus. This helps to prevent inadvertent waking and/or activation of the apparatus 2500, as might otherwise occur if the external station 2580 is moved with the apparatus 2500 disposed therein (e.g., for transport). The microcontroller 2572 is configured to determine whether the switch 2575 is in its first position or its second position. In this manner, the microcontroller 2572 is configured to determine whether the apparatus 2500 is disposed on the external station 2580.

The light therapy apparatus 2500 can also be configured to determine whether the mouthpiece 2510 is disposed within the patient's mouth (i.e., in a manner suitable for the treatment session). In this manner, the apparatus 2500 can be configured to only irradiate light for the treatment session when the apparatus 2500 has determined that the mouthpiece 2510 is disposed in the patient's mouth. In one or more embodiments, for example, the light therapy apparatus 2500 comprises a capacitance detection system configured to detect a capacitance change when the mouthpiece 2580 is disposed within the patient's mouth. Referring to FIGS. 93-96F, the capacitance detection system can comprise a capacitance sensor 2549 (see, e.g., FIG. 96F) disposed in the bill 2560 and configured to be in electrical communication with a first capacitance electrode 2550 and a second capacitance electrode 2552, each coupled to the flexible circuit board 2546 of the mouthpiece 2510. The flexible circuit board 2546 is configured to electrically couple the electrodes 2550, 2552 to the capacitance sensor 2549 in the bill 2560, such as via pathways 2543a-b and 2543c-d, respectively.

At least a portion of the sensors 2550, 2552 can be embedded in the flanges 2522, 2524 of the mouthpiece 2510, for example, in a similar manner as disclosed herein with respect to the light array 2542. The electrodes 2550, 2552 are spaced apart on the flexible circuit board 2546. In one or more embodiments, the electrodes 2550, 2552 are disposed at opposing locations with respect to the flexible circuit board 2546, as shown in FIG. 94, such that the first electrode 2550 is beneath the portion of the light array 2542 embedded in the first flange 2522 and the second electrode 2552 is beneath the portion of the light array 2542 embedded in the second flange 2524. In this manner, the apparatus 2500 is configured to detect a capacitance change bilaterally. In one or more embodiments, each electrode 2550, 2552 is extended posteriorly within the patient's mouth, thus providing for more “horizontal” contact with the patient's oral tissue for more robust sensing of the capacitance change.

The electrodes 2550, 2552 are configured to be disposed in close proximity to the patient's buccal tissue, which has a high capacitance, when the mouthpiece 2510 is disposed within the patient's mouth in preparation for treatment. The patient's saliva or wet buccal tissue can activate the capacitance of each electrode 2550, 2552. The apparatus 2500 is configured to irradiate light only after a predetermined capacitance change has been registered. Stated another way, the apparatus 2500, and the microcontroller 2572 more specifically, is configured to turn on the light emitters 2544 only after the predetermined capacitance change has been registered. The capacitance change is registered by the microcontroller 2572, which is configured to execute an algorithm to register the change in capacitance, when (1) the capacitance change threshold is detected by the capacitance sensor 2549 with respect to each electrode 2550, 2552 (i.e., bilaterally), and/or (2) the capacitance change is detected for a predetermined duration (e.g., for at least 2 seconds). When the microcontroller 2572 registers the capacitance change, a switch (also referred to herein as the “capacitance switch,” not shown in FIG. 96F, but generally included in the capacitance sensor 2549 components) in the bill 2560 and in electrical communication with the microcontroller 2572 is moved from a first position in which the light emitters 2544 are off, to a second position, in which the light emitters 2544 are on, thereby moving the apparatus 2500 to an active state in which the light emitters 2544 are irradiating light.

Because bilateral capacitance change is required to move the switch to its second position, the incidence of false positives that might result from the use of only one electrode is limited. In other embodiments, however, a light therapy apparatus can comprise only a single capacitance electrode. In yet other embodiments, a light therapy apparatus can comprise any other suitable detection mechanism for determining when the mouthpiece 2510 is positioned with the mouth.

Also, the capacitance detection system of the light therapy apparatus 2500 improves patient safety and/or compliance. For example, the light emitters 2544 can be configured to emit infrared light, so it is desirable to prevent emission of the light until the mouthpiece 2510 is properly disposed within the patient's mouth (e.g., to avoid the possibility of emitting radiation that could be harmful to the eyes). The arrangement of the capacitance sensor also eliminates the need for a manual “on/off” switch on the bill 2560. Such manual switches are prone to user error. For example, the patient might accidentally turn the light emitters off when the patient intended to begin a light therapy treatment session, as the patient might not readily discern whether the light emitters are off or on.

In one or more embodiments, the apparatus 2500 is configured to detect reflection of light from a patient's oral soft tissue. The light emitters 2544 can be configured to emit light, such as in a blinking or pulsing manner. The light emitters 2544 can be configured to blink or pulse at a predetermined rate. At least a portion of light emitted from the pulsing or blinking light emitters 2544 towards the oral soft tissue of the patient's mouth is reflected to the mouthpiece 2510 and is thereby detected by a sensor or other light detection mechanism (generally referred to as a “light sensor” or “photodetector,” not shown in FIGS. 84-97). The light sensor is configured to evaluate the functionality of a portion of the light array 2542 coupled to the first flange 2522 of the mouthpiece 2510 and a portion of the light array 2542 coupled to the second flange 2524 of the mouthpiece 2510. In this manner, the light sensor facilitates detection of any faulty operation of the apparatus 2500 with respect to each of the flanges 2522, 2544 of the mouthpiece 2510 before operation of the apparatus 2500 with respect to the patient. Suitable thresholds pertaining to the amount of light detected can be established, and used for example to assess whether the light emitters 2544 of the light array 2542 are operating properly. The apparatus 2500 can be configured to initiate irradiation of the oral tissue (e.g., begin a treatment session) when the light sensor detects the light reflection from the oral soft tissue and/or when the capacitance detection system detects a threshold capacitance change. In one or more embodiments, at least one of the light sensor or the capacitance detection system is configured to transmit a signal to the microcontroller 2572 in the bill 2560 to initiate the treatment session when the light sensor detects light reflected (e.g., at or above a predetermined threshold) from the oral soft tissue or when the capacitance sensor 2549 detects the threshold capacitance change.

The light sensor can also be configured to track the patient's compliance with a treatment program. For example, the light sensor can be configured to transmit a signal to the microcontroller 2572 one or more times that the light sensor detects the light reflection at a suitable threshold, which indicates that the mouthpiece 2510 was disposed in the patient's mouth. In this manner, the microcontroller 2572 can track the occurrences of when the patient placed the mouthpiece into the patient's mouth based on the signal transmitted by the light sensor.

In addition to controlling the operation of the apparatus 2500 based on movement and/or positioning within the mouth, the microcontroller 2572 can also control the operation of the apparatus 2500 and/or the light emitters 2544 based on the temperature of various regions of the apparatus and/or the anatomy of the patient. In particular, as disclosed herein, regulatory requirements and/or industry standards might set limits on the temperature of a medical apparatus in the mouth of a patient. The light therapy apparatus 2500 is configured to ensure the apparatus complies with an applicable regulatory requirement and/or industry standard. A temperature sensor 2577 (schematically shown in FIG. 96E), such as a thermocouple, is disposed in the bill 2560 and is configured to be in communication with the microcontroller 2572. The temperature sensor 2577 is coupled to one or more contacts 2545, 2547, disposed on the flexible circuit board 2546 of the mouthpiece 2510. As schematically shown in FIGS. 95 and 96E, the contacts 2545, 2547 can be coupled to the temperature sensor 2577 via pathways 2543e, 2543f.

The temperature sensor 2577 is configured to measure the temperature of the mouthpiece 2510 (e.g., via the contacts 2545, 2547) during a light therapy treatment session. The temperature sensor is configured to transmit an electrical signal associated with the measured temperature to the microcontroller 2572, or the like). In one or more embodiments, the position of the temperature sensor, or its contacts 2545, 2547, with respect to the flexible circuit board 2546 can vary from a position set forth in a regulation or industry standard. As such, the apparatus 2500 can be configured to execute a temperature algorithm (e.g., via the microcontroller 2572) that is configured to correct and/or adjust the temperature as measured by the temperature sensor based on the difference between the sensor's actual position and the position set forth in the regulation or standard, thereby calculating an adjusted temperature used to control the operation of the light therapy apparatus 2500.

In one or more embodiments, the light therapy apparatus 2500 is configured to temporarily cease light irradiation when a first predetermined temperature threshold is met or exceeded by at least one of the measured temperature or the adjusted temperature. The first predetermined temperature threshold can be a temperature sufficiently high to cause discomfort to the patient, but less than the regulatory (or industry standard) limit. In use, when the first predetermined temperature is met or exceeded, the light emitters 2544 are turned off, and the treatment session is paused. For example, the treatment session can be paused for a cooling period to permit the apparatus' 2500 temperature to drop to at least a predetermined temperature, or to a temperature lower than the first predetermined temperature threshold. For example, during a three minute treatment session, the cooling period can be about 20 seconds or about 30 seconds. If the temperature of the apparatus 2500 is sufficiently reduced, the light emitters 2544 are turned on (e.g., via the microcontroller 2572) and the treatment session is resumed.

In one or more embodiments, the light therapy apparatus 2500 is configured to cease light irradiation when a second predetermined temperature threshold, greater than the first predetermined temperature threshold, is met or exceeded by at least one of the measured temperature or the adjusted temperature. The second predetermined temperature threshold can be a temperature equivalent to the regulatory (or industry standard) limit. In one or more embodiments, the second predetermined temperature threshold ranges from about 45 degrees Celsius to about 55 degrees Celsius. In one or more embodiments, the second predetermined temperature threshold is about 48 degrees Celsius. If the second predetermined temperature threshold is met or exceeded, the light emitters 2544 are turned off, and the treatment session is ended. In such embodiments, the apparatus 2500 does not automatically resume the treatment program when the second predetermined temperature threshold is met or exceeded by at least one of the measured of adjusted temperature of the apparatus 2500.

The light therapy apparatus 2500 is configured to track a patient's compliance with a prescribed treatment program of light therapy treatment sessions. As such the apparatus 2500 is configured to store data associate with the patient's history of usage of the apparatus 2500. The apparatus 2500 can be configured to store data including one or more of (1) a total number of light therapy treatment sessions initiated and/or completed using the apparatus, (2) a total number of days the apparatus was used for administering light therapy treatment sessions, (3) whether the apparatus was used for administering light therapy to the upper or lower jaw (e.g., per treatment session), (4) a duration that light was administered by the apparatus for a particular date and time (e.g., if the patient completed less than a full treatment session, the apparatus can store the duration that light was administered), and (5) the date and time that light was administered using the apparatus 2500, or any combination of the foregoing.

In one or more embodiments, the light therapy apparatus 2500 is useful for treating a patient in combination with an orthodontic appliance, such as an aligner. In one or more embodiments, an aligner typically has a prespecified (e.g., by the manufacturer) duration of disposition that specifies the amount of time a user/patient should wear each aligner, usually for the purpose of effecting a predetermined and estimated amount of treatment (e.g., a prespecified amount of tooth movement affected by the aligner). In one or more embodiments, the use of the light therapy apparatus 2500 in combination with the aligner results in the use of the aligner for a first predetermined duration. During such use (i.e., during the first predetermined duration), the light therapy apparatus 2500 is useful for treating the patient for a second predetermined duration. In one or more embodiments, the same predetermined and estimated amount of orthodontic treatment is applied to the patient by this approach in the first predetermined duration as would have otherwise been applied using aligners alone in the disposition duration. In one or more embodiments, the disposition duration is about 18 hours, about 19 hours, about 20 hours, about 21 hours, about 22 hours, about 23 hours, including all values and subranges in between. In one or more embodiments, the first predetermined duration is about 7 hours, about 8 hours, about 9 hours, about 10 hours, about 11 hours, about 12 hours, about 13 hours, about 14 hours, about 15 hours, about 16 hours, about 17 hours, including all values and subranges in between. In one or more embodiments, the second predetermined duration is at least about 4 hours, at least about 5 hours, at least about 6 hours, at least about 7 hours, at least about 8 hours, at least about 9 about or at least about 10 hours, including all values, ranges and subranges in between. In one or more embodiments, the first predetermined duration is about 25% or less than the disposition duration, about 30% or less, about 35% or less, about 40% or less, about 45% or less, about 50% or less, about 55% or less, about 60% or less, about 65% or less, about 70% or less, about 70% or less, including all values and subranges in between.

The microcontroller 2572 is configured to transmit the stored data (i.e., the patient compliance information) to an external device. For example, the light therapy apparatus 2500 can be configured to transmit the patient compliance information to a mobile phone, personal digital assistant, computer, portable electronic device, the external station 2580, or the like. The apparatus 2500 can comprise a transmitter 2576 disposed in the bill 2560. In one or more embodiments, the transmitter 2576 is a wireless transmitter 2576 configured to wirelessly transmit data to the external device. The wireless transmitter 2576 can be configured to transmit the data via Bluetooth® or another suitable wireless mechanism and/or protocol. In one or more embodiments, the apparatus 2500 is configured for one-way transmission of data to the external device, e.g., via the transmitter 2576. In other embodiments, however, the transmitter is a transceiver, and therefore is configured for bi-directional communication of data with the external device. Bi-directional communication is useful for example for communication between the patient and his or her prescribing orthodontist.

As disclosed herein, the light therapy apparatus 2500 can have any suitable number of operational states or statuses. For example, in one or more embodiments, the light therapy apparatus 2500 can have a sleep state and a wake state. In the sleep state, the apparatus 2500 is prevented from irradiating light for a light therapy treatment session. The apparatus 2500 can be in a low power state during its sleep state, such that there is minimal electrical activity. Even in the sleep state, however, particular electronic components can continue to operate, such as a system clock (not shown). Additionally, in the sleep state, the apparatus 2500 can have a charge status or a communication status. In the charge status, the battery 2568 of the apparatus 2500 is being charged (or recharged), but the apparatus 2500 remains asleep. In the charge status, the wireless transmitter 2576, or other radio mechanism, of the apparatus 2500 is configured to wirelessly transmit information (e.g., patient compliance data) to the external device, however the apparatus remains in its sleep state.

In the wake state, the apparatus 2500 can be configured to have a ready status, an error status, a waiting status, an advertise status, an active status, a cooling status, a paused status, and a done status, or any combination thereof. In one or more embodiments, the apparatus 2500 is configured to be in its communication status when in the wake state instead of, or in addition to, when the apparatus is in the sleep state.

In the ready status, the apparatus 2500 is ready to begin irradiating light for the treatment session, but has not begun irradiating light. For example, in the ready status, the apparatus can be configured to begin the light therapy treatment session upon confirmation that the mouthpiece 2510 is properly disposed within the patient's mouth. In the error status, the apparatus 2500 has detected an error and is prevented from irradiating light for the treatment session. Like in the ready status, the apparatus 2500 in the waiting status is ready to begin irradiating light for the treatment session, but has not begun irradiating light. The apparatus 2500 is configured to enter the waiting status subsequent to the ready status, as described in more detail herein. In the advertise status, the apparatus 2500 is configured to produce an alert to the patient that the apparatus is ready for use in the treatment session. In the active status, the apparatus 2500 is irradiating light for the treatment session. In the cooling status, light irradiation from the apparatus 2500 is interrupted, or paused, for a predetermined period to permit the apparatus to cool down. In the paused status, light administration is temporarily ceased for a non-temperature related reason. In the done status, the apparatus 2500 has completed irradiating light for the treatment session, and therefore ceases light administration. In the communication status, the apparatus 2500 is configured to communicate with an external device.

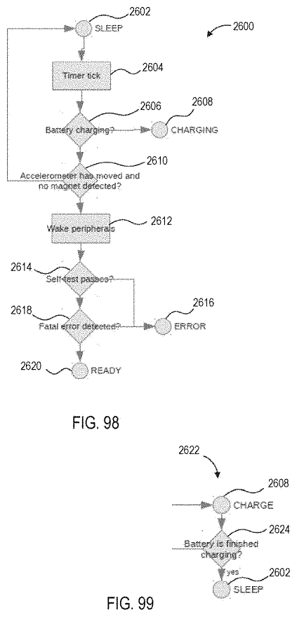

The apparatus 2500 (e.g., via the microcontroller 2572) is configured to execute various algorithms, including executing various logic sequences, to control the state or status of the apparatus. For example, as schematically illustrated in FIG. 98, the apparatus 2500 is configured to execute an algorithm 2600 for determining whether the apparatus can move from the sleep state to the wake state. Although the following descriptions refer to particular portions of the light therapy apparatus 2500, any of the methods described herein can be performed by any of the apparatuses described herein. Referring to FIG. 98, in the sleep state 2602, the system clock operates to keep time 2604. At 2606, the algorithm 2600 queries whether the battery 2568 is charging. If the battery 2568 is being charged, the apparatus 2500 enters the charge status 2608. If the battery 2568 is not charging, the algorithm 2600 queries, at 2610, (1) whether the accelerometer has moved (or detected three-dimensional movement of the apparatus 2500), and (2) whether no magnet (e.g., in the external station 2580) is detected. If the answer to either of query (1) or (2) is negative, i.e., that either the accelerometer has not moved (or detected three-dimensional movement) or that the magnet is detected, the apparatus 2500 remains in the sleep state. If the answer to both query (1) and (2) is affirmative, i.e., that the accelerometer has moved (or detected three-dimensional movement) and that no magnet is detected, peripheral electronics of the apparatus 2500 are awoken, at 2612. When the peripheral electronics are awake, the apparatus 2500 is generally referred to as being in its wake state (not shown in FIG. 98). The apparatus 2500 is then configured to perform a self-test to confirm the functionality of the peripheral electronics. The algorithm 2600 queries, at 2614, whether the apparatus 2500 passes the self-test. If the apparatus 2500 fails the self-test, the apparatus 2500 moves to the error status 2616. If the apparatus 2500 passes the self-test, the algorithm 2600 queries, at 2618, whether a fatal error is detected. If the fatal error is detected, the apparatus 2500 moves to the error status 2616. If the fatal error is not detected, the apparatus 2500 moves to the ready status 2620.

Referring to FIG. 99, in the charge status 2608, the apparatus 2500 (i.e., via the microcontroller 2572) is configured to execute an algorithm 2622 to query, at 2624, whether the battery 2568 has completed charging (e.g., is fully charged or is charged to at least a predetermined charge level). If charging the battery is incomplete, the apparatus 2500 remains in the charge status 2608. If the battery has completed charging, the apparatus 2500 returns to the (non-charging) sleep state 2602.

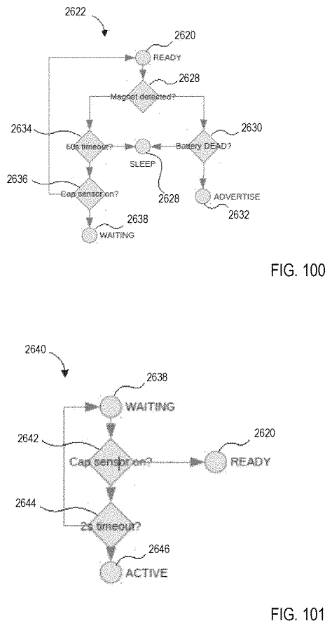

Referring to FIG. 100, in the ready status 2620, the apparatus 2500 is configured to execute an algorithm 2626 to query, at 2628, whether a magnet (e.g., in the external station 2580) is detected. The apparatus 2500 can determine if the magnet is detected based on whether the magnet switch in the bill 2560 is in its first position or its second position, as disclosed herein. If the magnet switch is in its second position, the microcontroller 2572 can determine that the electronic circuitry within which the magnet switch is disposed is complete, and thereby determining that the magnet is detected. If the magnet is detected, the algorithm queries, at 2630 whether the battery 2568 is low or depleted, and in need of charging. If the battery 2568 is low or depleted, the apparatus 2500 enters the advertise status 2632. The apparatus 2500 can be configured to alert the patient or other user that the battery 2568 is low or depleted, and in need of charging, when the apparatus is in the advertise status 2632. For example, in the advertise status, the apparatus 2500 can be configured to provide one or more of an audible indicia or a visual indicia (e.g., via a light color and/or pattern emitted by the light indicator 2578), as described herein. If the battery 2568 is not low or depleted, and therefore not in need of charging, the apparatus 2500 enters the sleep state 2602.

Returning to operation 2628, if the apparatus 2500 determines that the magnet is not detected, the algorithm 2626 then queries, at 2634, whether a predetermined period of time has elapsed. The predetermined period of time is intended to be a sufficient duration during which a patient can remove the apparatus 2500 from the external station 2580 and dispose the apparatus in the patient's mouth to begin a light therapy treatment session. Generally, the predetermined period of time is on the order of seconds or minutes. For example, the predetermined period can be 20 seconds, 30 seconds, 40 seconds, 50 seconds, 60 seconds, 70 seconds, or more. In one or more embodiments, the predetermined period of time is 60 seconds. If the predetermined period of time has elapsed, the apparatus 2500 enters the sleep state 2602. In this manner, the apparatus 2500 is configured to help prevent depletion of the battery 2568 due to the apparatus maintaining itself the ready status 2620 beyond the predetermined period of time. If the predetermined period of time has not elapsed, the algorithm 2626 queries, at 2636, whether the capacitance sensor 2549 is on. Stated another way, the algorithm 2626 queries whether the capacitance sensor 2549 indicates that the electrodes 2550, 2552 are, at the time of the query, detecting a capacitance change. If the capacitance sensor 2549 is on, the apparatus 2500 enters the waiting status 2638. If the capacitance sensor 2549 is not on (stated another way, if at least one of the capacitance electrodes 2550, 2552 does not, at the time of the query, detect a capacitance change, i.e., the sensor 2549 is “off”), the apparatus 2500 remains in the ready status 2620. If the apparatus 2500 remains in the ready status 2620, the algorithm 2626 can be reexecuted until the apparatus enters a different status, such as one of the advertise 2632, sleep 2602, or waiting 2638 statuses.

Referring to FIG. 101, in one or more embodiments, the apparatus 2500 is configured to execute an algorithm 2640 to determine whether the apparatus 2500 has been properly disposed within the patient's mouth. Such information can be conveyed to for example, the patient's prescribing physician via the transmitter 2576. More specifically, when the apparatus 2500 is in the waiting status 2638, the apparatus is configured to execute an algorithm 2640 to query, at 2642, whether the capacitance sensor 2549 is on (e.g., whether the sensor 2549 has detected a capacitance change). In one or more embodiments, the capacitance sensor 2549 is configured to turn on when a capacitance change detected by each capacitance electrode 2550, 2552 is at or exceeds a predetermined threshold. The query, at 2642, is intended to determine whether the apparatus 2500 has been disposed within the patient's mouth in preparation for administration of the light therapy treatment session. If at least one of the capacitance electrodes 2550, 2552 has not detected a capacitance change at or exceeding the predetermined threshold, the capacitance sensor 2549 is off and the apparatus 2500 returns to the ready status 2620.

If it is determined that the capacitance sensor 2549 is on, the algorithm 2640 queries, at 2644, whether a predetermined period of time has elapsed subsequent to the determination that the capacitance sensor 2549 is on. The query, at 2642, is intended to determine whether the apparatus 2500 is properly positioned within the patient's mouth, thereby ensuring substantially balanced light administration to each side of the patient's mouth (e.g., with the light energy difference between the sides being no more than plus or minus about 5%). Generally, the predetermined period of time is a duration on the order of seconds. For example, the predetermined period of time can be a duration ranging from about 1 second to about 5 seconds. In one or more embodiments, the predetermined period of time is a duration of 2 seconds. If the algorithm 2640 determines that the capacitance sensor 2549 was not on for the predetermined period of time, the apparatus 2500 remains in the waiting status 2638. If the capacitance sensor 2549 remained on for the predetermined period of time, the apparatus 2500 enters the active status 2646. In one or more embodiments, the microcontroller 2572 registers the bilateral detection of the capacitance change at or exceeding the predetermined threshold for the predetermined period of time.

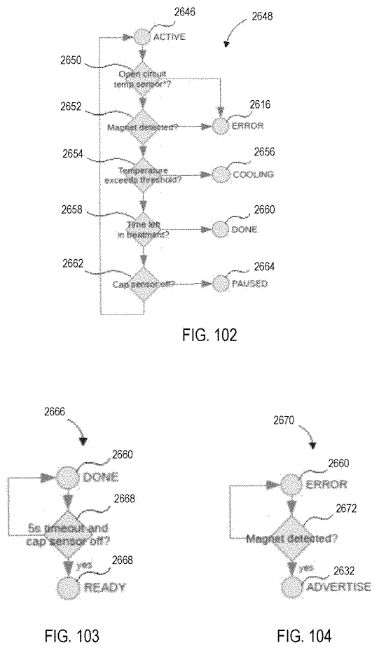

Referring to FIG. 102, when in the active status 2646, the apparatus 2500 is configured to execute an algorithm 2648 to query, at 2650, whether the temperature sensor 2577 (e.g., the thermocouple) is detected or functional. For example, the temperature sensor 2577 can indicate to the microcontroller 2572 that it is functional if the temperature sensor confirms it is able to determine a temperature based on information received from the one or more contacts 2545, 2547 on the flexible circuit board 2546. If the temperature sensor 2577 is not detected or is not functional, the apparatus 2500 enters the error status 2616. If it is determined that the temperature sensor 2577 is detected, or is functional, the algorithm 2648 is configured to query, at 2652, whether the magnet is detected. If it is determined that the magnet is detected, the apparatus 2500 enters the error status 2616. As can be understood in view of the description regarding apparatus 2500 herein, the apparatus 2500 is not intended to be in the active status (in which light can be emitted) and concurrently disposed on the external station 2580 such that the magnet can be detected.

If, at 2652, it is determined that the magnet is not detected, the algorithm 2648 is configured to query, at 2654, whether a temperature of the apparatus 2500 exceeds a predetermined temperature threshold. As disclosed herein, in one or more embodiments, the temperature of the apparatus 2500 is the temperature measured by the temperature sensor 2577. In other embodiments, the temperature of the apparatus 2500 is the adjusted temperature calculated by the microcontroller 2572 based on the measured temperature. If it is determined that the temperature exceeds the predetermined temperature threshold, the apparatus 2500 enters the cooling status 2656.

If it is determined that the temperature does not exceed (i.e., is at or below) the first predetermined temperature threshold, the algorithm 2648 is configured to query, at 2658, whether any time remains for administering the treatment session. As disclosed herein, for example, a treatment session can be about 3 minutes in duration. As such, the algorithm 2648 is configured to determine whether any of the 3 minutes for the treatment session remains, i.e., has not yet been completed. If it is determined that no time remains in the treatment session, the apparatus enters the done status 2660.

If it is determined that time remains in the treatment session, the algorithm 2648 is configured to query, at 2662, whether the capacitance sensor 2549 is off. If the capacitance sensor 2549 is off, the apparatus 2500 enters the paused status 2664. The capacitance sensor 2549 might have turned off, for example, because the patient removed the mouthpiece 2510 from the patient's mouth, or inadvertently shifted the mouthpiece 2510 within the patient's mouth such that at least one electrode 2550, 2552 does not have the threshold capacitance charge. If the capacitance sensor 2549 is on, the apparatus 2500 remains in the active status 2646, and the algorithm 2648 is reexecuted. The algorithm 2648 can be configured to be automatically reexecuted until the apparatus 2500 enters a status other than the active status 2646, such as one of the error status 2616, cooling status 2656, done status 2660, or paused status 2664.

Referring to FIG. 103, when the apparatus 2500 is in the done status 2660, the apparatus 2500 is configured to execute an algorithm 2666 configured to determine if the apparatus should enter the ready status. Specifically, the algorithm 2666 is configured to query, at 2668, whether a predetermined period of time has elapsed since the apparatus 2500 entered the done status 2660. The predetermined period of time can be on the order of seconds, such as, for example, two seconds, three second, four seconds, five seconds, six seconds, or more. In one or more embodiments, the algorithm 2666 is configured to query whether 5 seconds have elapsed since the apparatus 2500 entered the done status. The algorithm 2666 is also configured to query whether the capacitance sensor 2549 is off. The algorithm can be configured to execute both queries, i.e., whether the predetermined period of time has elapsed and whether the capacitance sensor 2549 is off, concurrently. If it is determined that (1) the predetermined period of time has elapsed, and (2) the capacitance sensor 2549 is off, the apparatus 2500 enters the ready status 2620. If either the predetermined time period has not elapsed or the capacitance sensor 2549 is on, the apparatus 2500 remains in the done status 2660, and the apparatus 2500 can be configured to reexecute the algorithm 2666 until the apparatus enters a different status (e.g., the ready status 2620).

Referring to FIG. 104, when the apparatus 2500 is in the error status 2616, the apparatus 2500 is configured to execute an algorithm 2670 to determine if the apparatus should enter a different status, such as the advertise status 2632. The algorithm 2670 is configured to query, at 2672, whether the magnet is detected. If it is determined that the magnet is detected (e.g., if the apparatus 2500 is disposed on the external station 2580), the apparatus 2500 enters the advertise status 2632. If it is determined that the magnet is not detected, the apparatus 2500 remains in the error status 2616. The apparatus 2500 can be configured to reexecute the algorithm 2670 until the apparatus enters a different status (e.g., the advertise status 2632).

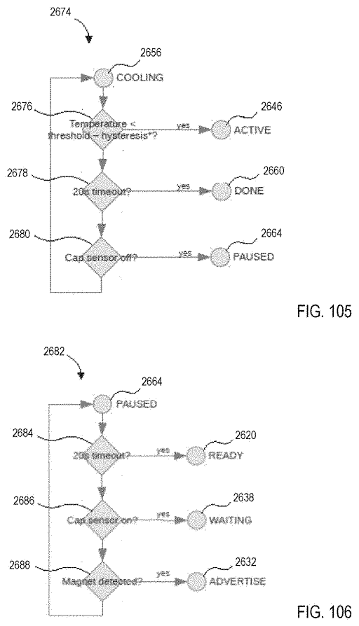

As disclosed herein, when the apparatus 2500 is in the cooling status, light emission from the light array 2542 during a treatment session is, at least temporarily, ceased to permit the apparatus to cool down. The apparatus 2500 can be configured to execute an algorithm to determine if the apparatus should move from the cooling status 2656 to a different status, such as the active status 2646, done status 2660, or paused status 2664. Referring to FIG. 105, the apparatus 2500 is configured to execute an algorithm 2674 to query, at 2676, whether the temperature of the apparatus is less than the predetermined temperature threshold. More specifically, in one or more embodiments, the algorithm 2674 is configured to query, at 2676, whether the temperature of the apparatus 2500 is less than the predetermined temperature threshold value minus a hysteresis value. In the cooling status 2656, temperature of the apparatus 2500 should continue lowering until the apparatus 2500 reaches an ambient temperature of its environment. As such, a real-time temperature of the apparatus 2500 at the moment the apparatus determines an answer to the query, at 2676, might be less than the measured (or adjusted) temperature used to determine the answer to the query. The hysteresis value is a value used to adjust for the time lag between the temperature being measured (or adjusted) and the execution of the algorithm. If it is determined that the temperature of the apparatus 2500 is less than the predetermined temperature threshold, and in one or more embodiments less than the predetermined temperature threshold minus the hysteresis value, the apparatus enters (or returns) to the active status 2646.

If it is determined that the temperature of the apparatus 2500 is not less than the predetermined temperature threshold, and, in one or more embodiments is not less than the predetermined temperature threshold minus the hysteresis value, the algorithm 2674 is configured to query, at 2678, whether a predetermined time period has elapsed since the apparatus 2500 entered the cooling status 2656. The predetermined time period can be on the order of seconds, for example ranging from about 5 seconds to about 30 seconds. In one or more embodiments, the predetermined time period is 20 seconds. If it is determined that the predetermined time period has elapsed, the apparatus 2500 enters the done status 2660.

If it is determined that the predetermined time period has not elapsed, the algorithm 2674 is configured to query, at 2680, whether the capacitance sensor 2549 is off. If it is determined that the capacitance sensor 2549 is off, the apparatus 2500 enters the paused status. If it is determined that the capacitance sensor 2549 is on, the apparatus 2500 remains in the cooling status 2656. The apparatus 2500 can be configured to reexecute the algorithm 2674 until the apparatus enters a different status, such as the active status 2646, the done status 2660, or the paused status 2664.

The apparatus 2500 can be configured to determine whether the apparatus, when in the paused status 2664, should enter a different status, such as one of the ready status 2620, the waiting status 2638, or the advertise status 2632. Referring to FIG. 106, the apparatus 2500 can be configured to execute an algorithm 2682, when the apparatus is in the paused status 2664, configured to query, at 2684, whether a predetermined period of time has elapsed since the apparatus 2500 entered the paused status. The predetermined time period can be on the order of seconds, for example ranging from about 5 seconds to about 30 seconds. In one or more embodiments, the predetermined time period is 20 seconds. If it is determined that the predetermined time period has elapsed, the apparatus 2500 enters the ready status 2620. If it is determined that the predetermined time period has not elapsed, the algorithm 2682 is configured to query, at 2686, whether the capacitance sensor 2549 is on. If it is determined that the capacitance sensor 2549 is on, the apparatus 2500 enters the waiting status 2638. If it is determined that the capacitance sensor 2549 is not on, the algorithm is configured to query, at 2688, whether the magnet is detected. If it is determined that the magnet is detected, the apparatus 2500 enters the advertise status 262. If it is determined that the magnet is not detected, the apparatus 2500 remains in the paused state 2664. The apparatus 2500 can be configured to reexecute the algorithm 2682 until the apparatus enters a different status, such as the ready status 2620, the waiting status 2638, or the advertise status 2632.

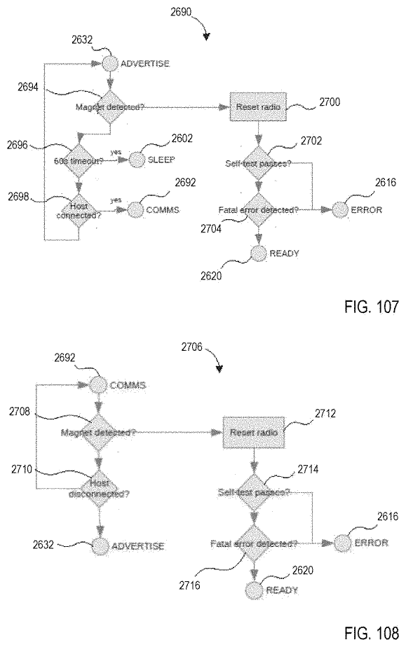

The apparatus 2500 can be configured to determine whether the apparatus, when in the advertise status 2632, should enter a different status, such as one of the sleep status 2602, communication status 2692, error status 2616, or ready status 2620. Referring to FIG. 107, the apparatus 2500 can be configured to execute an algorithm 2690, when the apparatus is in the advertise status 2632, configured to query, at 2694, whether the magnet is detected. As disclosed herein, the magnet can be detected, for example, if the apparatus 2500 is disposed on the external station 2580. If it is determined that the magnet is detected, the algorithm 2690 is configured to query, at 2696 whether a predetermined period of time has elapsed since the apparatus 2500 entered the advertise status 2632. The predetermined time period can be on the order of seconds or minutes, for example ranging from about 20 seconds to about 2 minutes. In one or more embodiments, the predetermined time period ranges from about 45 seconds to about 75 seconds. In one or more embodiments, the predetermined time period is 60 seconds. If it is determined that the predetermined time period has elapsed, the apparatus 2500 enters the sleep status 2602. If it is determined that the predetermined time period has not elapsed, the algorithm 2690 queries, at 2698, whether the external device (also referred to as a host device, e.g., a mobile phone, personal digital assistant, computer, portable electronic device, or the like) is connected to, or is in communication with, the apparatus 2500. If it is determined that the external device is connected to, or is in communication with, the apparatus 2500, the apparatus enters the communication status 2692. If it is determined that the external device is not connected to, or is not in communication with, the apparatus 2500, the apparatus remains in the advertise status 2632. The apparatus 2500 can then reexecute the algorithm 2690.

Returning to the algorithm's query, at 2694, of whether the magnet is detected, if the magnet is not detected, the apparatus 2500 is configured to reset its wireless transmitter, or other radio mechanism, as indicated at 2700. The algorithm 2690 is configured to query, at 2702, whether the apparatus 2500 passes a self-test. In one or more embodiments, as part of the query, the algorithm 2690 can be configured to execute the self-test. In other embodiments, the algorithm 2690 can be configured to cause the apparatus 2500 to execute a different algorithm (not shown) for performing the self-test. The self-test can be configured to determine whether the wireless transmitter 2576, or other radio mechanism, of the apparatus 2500 is functional. If it is determined that the apparatus 2500 did not pass the self-test, the apparatus enters the error status 2616. If it is determined that the apparatus 2500 passes the self-test, the algorithm 2690 is configured to query, at 2704, whether a fatal error is detected. If it is determined that a fatal error is detected, the apparatus 2500 enters the error status 2616. If it is determined that a fatal error is not detected, the apparatus 2500 enters the ready status 2620. The apparatus 2500 can be configured to reexecute at least a portion of the algorithm 2690 until the apparatus enters a different status, such as the sleep status 2602, communication status 2692, error status 2616, or ready status 2620.

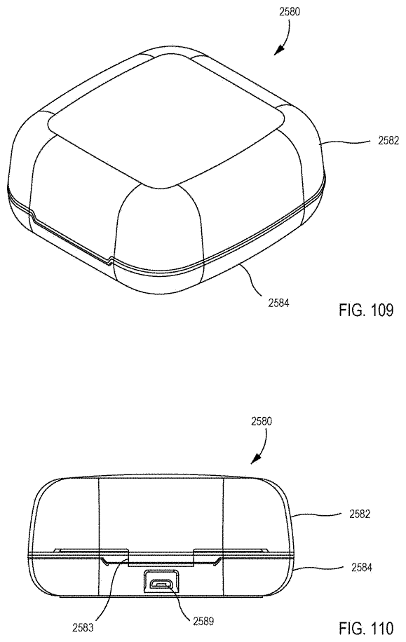

The apparatus 2500 can be configured to determine whether the apparatus, when in the communication status 2692, should enter a different status, such as one of the advertise status 2632, error status 2616, or ready status 2620. Referring to FIG. 108, the apparatus 2500 can be configured to execute an algorithm 2706, when the apparatus is in the communication status 2692, configured to query, at 2708, whether the magnet is detected. As disclosed herein, the magnet can be detected, for example, if the apparatus 2500 is disposed on the external station 2580. If it is determined that the magnet is detected, the algorithm 2706 is configured to query, at 2710, whether the external device (also referred to as a host device, e.g., a mobile phone, personal digital assistant, computer, portable electronic device, or the like) is disconnected from, or is not in communication with, the apparatus 2500. If it is determined that the external device is disconnected from, or is not in communication with, the apparatus 2500, the apparatus enters the advertise status 2632. If it is determined that the external device is not disconnected from, or is in communication with, the apparatus 2500, the apparatus remains in the communication status 2692. The apparatus 2500 can then reexecute the algorithm 2706.

Returning to the algorithm's query, at 2708, of whether the magnet is detected, if the magnet is not detected, the apparatus 2500 is configured to reset its wireless transmitter, or other radio mechanism, as indicated at 2712. The algorithm 2706 is configured to query, at 2714, whether the apparatus 2500 passes the self-test. In one or more embodiments, the algorithm 2706 can be configured to execute the self-test. In other embodiments, the algorithm 2706 can be configured to cause the apparatus to execute a different algorithm (not shown) for performing the self-test. As described herein, the self-test can be configured to determine whether the wireless transmitter 2576, or other radio mechanism, of the apparatus 2500 is functional. If it is determined that the apparatus 2500 did not pass the self-test, the apparatus enters the error status 2616. If it is determined that the apparatus passes the self-test, the algorithm 2706 is configured to query, at 2716, whether a fatal error is detected. If it is determined that a fatal error is detected, the apparatus 2500 enters the error status 2616. If it is determined that a fatal error is not detected, the apparatus 2500 enters the ready status 2620. The apparatus 2500 can be configured to reexecute the algorithm 2706 until the apparatus enters a different status, such as the advertise status 2632, error status 2616, or ready status 2620.

Although the algorithms (e.g., algorithms 2600, 2622, 2626, 2640, 2648, 2666, 2670, 2674, 2682, 2690, 2706) have been illustrated and described herein as executing particular queries or steps in a particular order, in one or more embodiments, particular queries or steps can be differently ordered and/or particular queries or steps can be omitted. For example, in one or more embodiments, the algorithm query regarding whether the apparatus 2500 passes a self-test can be omitted. In another example, in one or more embodiments, the algorithm can query whether the capacitance sensor 2549 is on (or off, as applicable for a particular query), after (e.g., with respect to the waiting status algorithm) or before (e.g., with respect to the ready status algorithm) the query regarding whether a predetermined period of time has elapsed.

Furthermore, although the algorithms (e.g., algorithms 2600, 2622, 2626, 2640, 2648, 2666, 2670, 2674, 2682, 2690, 2706) have been illustrated and described herein as being distinct algorithms, in one or more embodiments, the executable code represented by the algorithms described herein can be included in a single algorithm. In other embodiments, the executable code represented by the algorithms described herein can be included in two or more algorithms. Moreover, the apparatus 2500 can be configured to execute algorithms in addition to those illustrated and described herein. For example, the apparatus 2500 can be configured to execute an algorithm configured to permit a maximum of two treatment sessions to be administered by the apparatus 2500 per day. In another embodiment, the apparatus 2500 can be configured to permit a maximum of four treatments sessions per day (e.g., two treatment sessions per upper and lower arch, per day.

In one or more embodiments, the light therapy apparatus 2500 is configured to provide indicia of a state or status of the apparatus. The term “indicia,” is used herein as including the singular (“indicium”) or the plural (“indicia”), unless the context clearly indicates otherwise. The indicia can include one or more of an audible indicia (e.g., a tone, beep, announcement, or the like), a tactile indicia (e.g., a vibration or the like), or a visual indicia (e.g., a light, a displayed message, or the like). The bill 2560 comprises an indicator light 2578 configured to emit at least one of a pattern of light or a color of light, or both, indicative of the status of the light therapy apparatus 2500 or a treatment session being administered by the apparatus. More specifically, for example, the indicator light 2578 is configured to indicate, based on a combination of (1) the color of the light being emitted and (2) whether the light is being emitted in a solid pattern, a slow blink pattern or a fast blink pattern, a status of the apparatus. The indicator light 2578 can be or comprise a light pipe.

For example, the indicator light 2578 can be configured to emit no light when the apparatus 2500 is turned off. In one or more embodiments, a patient can be instructed to return the apparatus 2500 to the external station 2580 when no light is emitted by the indicator light 2578. The indicator light 2578 can be configured to emit a green, solid light when the apparatus 2500 is ready to begin a treatment session. The indicator light 2578 can be configured to emit a green, fast blink light when the apparatus 2500 is ready to begin a treatment session and should be recharged after the treatment session. The indicator light 2578 can be configured to emit a green, slow blink light when the apparatus 2500 is being charged and/or when the apparatus 2500 is in wireless communication with an application of the external device.

The indicator light 2578 can be configured to emit a blue, solid light when the apparatus 2500 is activated and providing a treatment session. The indicator light 2578 can be configured to emit a blue, fast blink light when the treatment session is paused, but the treatment session can be continued. The indicator light 2578 can be configured to emit a blue, slow blink light when the treatment session is complete and the apparatus 2500 should be removed from the patient's mouth. The indicator light 2578 can be configured to emit a red, blinking light when the battery 2568 is low, indicating that the apparatus 2500 should be returned to the external station 2580 for recharging. The indicator light 2578 can be configured to emit a red, solid light when the apparatus 2500 is has detected an error, indicating that the reset protocol for the apparatus 2500 should be followed by the patient, which can comprise placing the apparatus 2500 on the external station 2580 and/or contacting customer support.

In one or more embodiments, the apparatus 2500 is configured to provide the audible indicia to the patient. For example, in one or more embodiments, the apparatus 2500 is configured to beep to alter the patient that at least one of (1) a treatment session has begun, (2) a treatment session has been paused, (3) the treatment session has ended.

Although the indicator light 2578 has been described as being configured to emit a specific color and pattern (e.g., green, solid) for a specific status (e.g., ready to begin treatment), in other embodiments, the apparatus 2500 can be programmed to cause the indicator light 2578 to emit light in any suitable combination of color and pattern for a variety of statuses.

The light therapy apparatus 2500 can be configured for use in an orthodontic treatment, including any treatment described herein.

In use, upon removal from its case, the light therapy apparatus 2500 is configured to automatically wake up from the sleep state, and go into a ready state (or pre-treatment mode), as described herein. In the ready state, the apparatus 2500 will periodically check for tissue contact. The patient places the mouthpiece 2510 into their mouth and positions the apparatus 2500 comfortably to begin treatment. Correct positioning is achieved by centering the mouthpiece 2510 in the mouth and setting the ridge 2518 in between the patient's central incisors. Once the apparatus 2500 is positioned correctly, the patient will bite down on the bite pad 2514. The light therapy apparatus 2500 detects when tissue contact has been achieved, and will automatically begin treatment, as described in more detail herein.

In one or more embodiments, for example, the light therapy apparatus 2500 is useful to irradiate at least a portion of the patient's upper jaw for about 3 minutes, the patient's lower jaw for about 3 minutes, or each of the patient's upper and lower jaws for about 3 minutes. More specifically, in one treatment program, the light therapy apparatus 2500 is useful to administer a light-therapy treatment session in which the oral tissue associated with each of the upper arch of the patient's mouth and the lower arch of the patient's mouth (or vice versa) are consecutively irradiated for 3 minutes per day, for a total treatment session of 6 minutes per day. During the treatment session, the light can be irradiated concurrently from all light emitters 2544 in the light array 2542. Also during treatment, the light emitters 2544 can include embedded LEDs configured to activate and directly irradiate the alveolar mucosa and alveolar bone. Administration of a first light therapy session of the treatment program can begin on the same date that the patient's orthodontic treatment begins (e.g., the day that brackets and wires are installed on the patient's teeth, e.g., T0).

The light can be emitted at any suitable wavelength or combinations of wavelengths in accordance with the methods described herein. In one or more embodiments, the light is emitted at a wavelength of about 850 nm during the treatment session. In other embodiments, the light is emitted at a wavelength of 850 nm (f 5 nm) during the treatment session. In one or more embodiments, the light emitters 2544 and/or LEDs can emit light at a blend of wavelengths, and not at a single wavelength like a laser. The peak light emission wavelength (λmax) by the LEDs, can be, for example, 855 nm.

The treatment sessions can be administered for any suitable period, including, but not limited to, a period of four to twelve months. In one or more embodiments, use of the light therapy apparatus 2500 to administer light therapy treatment sessions according to a treatment program is continued until the patient's Little's Irregularity Index (“LII”) score, described in more detail herein, is determined to be less than 1 (i.e., T1). The patient's treatment program can continue until the patient's dental malocclusion is completely resolved (e.g. the patient's LII is about zero) and/or an acceptable clinical outcome has been achieved, which, in one or more embodiments, is determined at the patient's final orthodontic appointment during which the orthodontic appliance (e.g., brackets and wires) are uninstalled. In one or more embodiments, use of the light therapy apparatus 2500 to administer light therapy treatment sessions according to a treatment program is continued until the patient's orthodontic appliance is uninstalled from the patient's teeth (i.e., T2). The light therapy apparatus 2500 can be configured to administer the treatment sessions in a clinical setting or in a home environment.

Such a treatment program can, for example, reduce the duration of an average period a patient is expected to need to use an orthodontic appliance (e.g., braces) to achieve a desired orthodontic result. For example, the apparatus 2500 can be useful during the treatment program to reduce the duration of the treatment program from two years to six months. The foregoing treatment program and/or any treatment program described herein can reduce a duration of an orthodontic treatment administered without light therapy, as described herein, by about 50 percent to about 75 percent.

Referring to FIGS. 109-113, the light therapy apparatus 2500 can be configured to be disposed on, in or otherwise coupled to the external station 2580, for example, when the apparatus 2500 is not in use by the patient. The external station 2580 can be, for example a carrying case, charging caddy or station, or the like, or a combination of the foregoing.

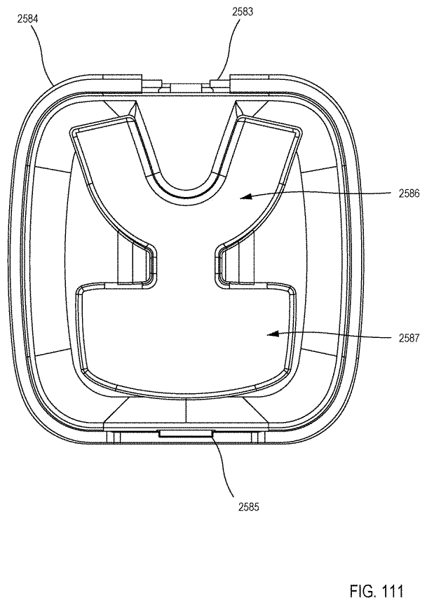

The station 2580 comprises a base 2584 and a lid 2582 and defines a cavity (not shown in FIGS. 109-113) formed by and between the base 2584 and the lid 2582 when the lid is in a closed position (as shown in FIG. 109). The lid 2582 can be coupled to the base 2584 using any suitable coupling mechanism, for example, using a hinge 2583 as shown in FIGS. 110 and 111. In this manner, the lid 2582 is conveniently moveable between its closed position and an open position (not shown).

The base 2584 can comprise a locking mechanism configured to secure the lid 2582 to the base 2584. For example, the base can comprise a latch 2585 configured to matingly engage a recess (not shown) in the lid 2582 to secure the lid in the closed position. The base 2584 and lid 2582 can be constructed of any suitable material, including, for example a plastic such as Bisphenol A-free polypropylene. In one or more embodiments, at least one of the base 2584 and lid 2582 is constructed of a material that is resistance to impact and/or scratches.

The base 2584 can define a first recess 2586 configured to receive at least a portion of the mouthpiece 2510 and a second recess 2587 configured to receive at least a portion of the bill 2560. In one or more embodiments, as shown in FIGS. 111 and 112, the first recess 2586 is in continuous with the second recess 2587. A perimeter of the first recess 2586 can, for example, complement the contour of the mouthpiece 2510. A perimeter of the second recess 2587 can, for example, complement the contour of the bill 2560.

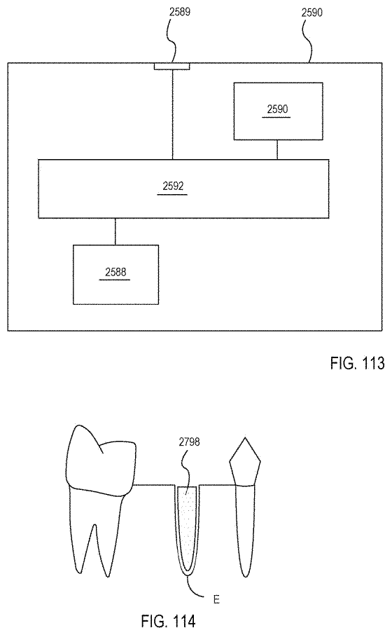

The external station 2580 can be configured to charge the apparatus 2500 when the apparatus 2500 is disposed on or otherwise coupled to the station. In this manner, the battery 2568 can be recharged when the bill 2560 is coupled to the charging station. In one or more embodiments, for example, the station 2580 is configured to inductively charge the apparatus 2500, e.g., by inductively charging the battery 2568 via the induction receiver coil 2569 of the mouthpiece 2510. More specifically, an electronics assembly 2590 (schematically illustrated in FIG. 113) comprises a wireless charger (e.g., an induction transmitter coil) 2588 configured to induce an electromagnetic field in the induction receiver coil 2569, and the induction receiver coil is configured to convert the received energy into an electrical current for charging the battery 2568.

In one or more embodiments, the external station 2580 is configured to be coupled to a computer or other electronic device, such as via a USB, micro-USB or other suitable cable (not shown) received in a port 2589 of the external station 2580. For example, the USB cable can be configured for charging the external station 2580. In other embodiments, the external station 2580 can be configured to be charged by any conventional power supply. In one or more embodiments, external station 2580 is configured to be coupled to a medical grade power supply, such as a medical grade 6 W power supply manufactured by TRUMPower. The power supply can be configured to comply with applicable regulatory standards and/or regulations. For example, the power supply can be configured to comply with IEC 60601-1 and/or other applicable standards. Electronic circuitry 2592 is configured to couple the port 2589 to the induction transmitter coil 2588, thus facilitating the inductive charging of the apparatus 2500 with no electrical connections between the apparatus 2500 and the external station 2580.

In one or more embodiments, the external station 2580 is configured to sanitize or otherwise disinfect at least a portion of the light therapy apparatus 2500. For example, the external station 2580 can comprise a light emitter 2594 configured to emit an ultraviolet light, or other suitable wavelength light, such as a blue light, to disinfect the mouthpiece 2510 when the apparatus 2500 is disposed in the external station 2580. In one or more embodiments, the lid 2582 of the external station 2580 must be in the closed position for the station to emit the disinfecting light. For example, in one or more embodiments, when the lid 2582 is in its closed position, a switch (not shown) is moved to a closed position, thereby completing an electrical circuit including the light emitter 2594 such that the light emitter 2594 can irradiate the light. In one or more embodiments, emission of the disinfecting light can be controlled via the electronics assembly 2590 of the external station 2580.

The external station 2580 comprises a magnet 2596 (schematically illustrated with phantom lines in FIG. 112). As described herein, the magnet 2596 is configured to cause the magnet switch 2575 of the bill 2560 to move from its first position to its second position when the apparatus 2500 is disposed on the base 2584 of the external station 2580.

Although the light therapy apparatus 2500 and external station 2580 have been described herein as being configured to comply with various industry standards, the apparatus 2500 and/or external station 2580 can be configured to comply with additional or alternative industry standards. For example, the apparatus 2500 and external station 2580 can be configured to be compliant with one or more of the following standards, or any combination thereof: IEC/EN 60601-1 Ed. 3.1: 2012—Medical Electrical Equipment Part 1: General requirements for basic safety and essential performance; IEC/EN 60601-1-2 Ed. 3: 2007—Collateral standard: Electromagnetic compatibility—Requirements and tests; EN 62471: 2009—Photobiological safety of lamps and lamp systems; IEC 60601-2-57: Ed. 1.0: 2011—Medical Electrical Equipment Part 2-57: Particular requirements for the basic safety and essential performance of non-laser light source equipment intended for therapeutic, diagnostic, monitoring and cosmetic/aesthetic use; EN 60529 Ed. 2.1: 2001—Degrees of protection provided by enclosures; IEC 60601-1-11—Collateral Standard: Requirements for medical electrical equipment and medical electrical systems used in the home healthcare environment; ISO 10993-1: 2009—Biological evaluation of medical devices—Part 1: Evaluation and testing within risk management process; ISO/BS/EN 14971: 2012—Medical Devices—Application of risk management to medical devices.

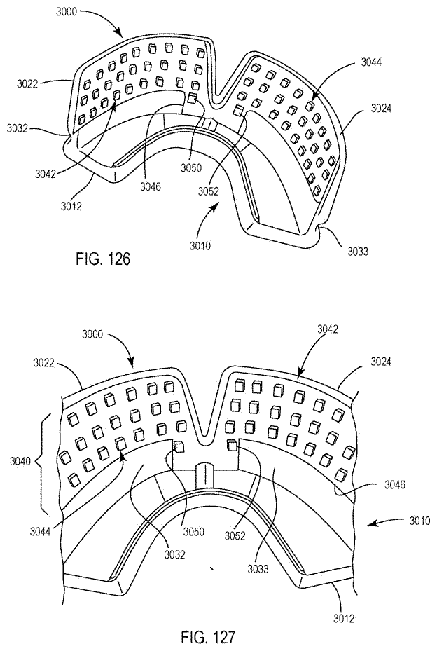

Although light therapy apparatus have been shown and described herein (e.g., light therapy apparatus 2500) as comprising a capacitance detection system to determine whether a mouthpiece (e.g., mouthpiece 2510) is disposed within the patient's mouth (i.e., in a manner suitable for the treatment session), in other embodiments, a light therapy apparatus can comprise a different mechanism to determine whether the mouthpiece is disposed within the patient's mouth. As shown in FIGS. 126-127, in one or more embodiments a light therapy apparatus 3000 comprises a mouthpiece 3010 configured to be disposed within a mouth of a patient. The light therapy apparatus 3000 can be similar, or identical, to light therapy apparatus 2500, and mouthpiece 3010 can be similar to mouthpiece 2510, except as described herein. For example, mouthpiece 3010 comprises a bite tray 3012, flanges 3022, 3024 transversely coupled to the bite tray, an electronics assembly 3040 including a light array 3042 and a flexible circuit board 3046, each of which is similar to a corresponding component of mouthpiece 2510, and thus is not described in detail with respect to mouthpiece 3010. In one or more embodiments, at least one or more portions of the mouthpiece 3010 are constructed from a substantially transparent material (e.g., silicone) such that one or more components embedded within the mouthpiece 3010 are visible through the mouthpiece 3010. Thus, for purposes of illustration, portions of the mouthpiece 3010, including portions of the first flange 3022, the second flange 3024 and the bite tray 3012 are shown as being transparent in FIGS. 126 and 127 to show portions of the electronics assembly 3040 and the structure disposed therein.

The light therapy apparatus 3000 is configured to determine whether the mouthpiece 3010 is disposed within the patient's mouth (i.e., in a manner suitable for a treatment session, as described herein). In this manner, the light therapy apparatus 3000 can be configured to only irradiate light for the treatment session when the apparatus 3000 has determined that the mouthpiece 3010 is disposed in the patient's mouth. The mouthpiece 3010 comprises a first light emitter 3050 and a second light emitter 3052, each of which is configured and positioned to detect or measure an amount of light reflected by a portion of the patient's oral tissue. For example, the first and second light emitters 3050, 3052 can be configured to detect light emitted from the light emitters 3044 of the light array 3042 and reflected by the patient's oral soft tissue. In one or more embodiments, the light emitters 3044 can be configured to emit light, such as in a blinking or pulsing manner. The light emitters 3044 can be configured to blink or pulse at a predetermined rate. At least a portion of light emitted from the pulsing or blinking light emitters 3044 towards the oral soft tissue of the patient's mouth is reflected to the mouthpiece 3010 and is thereby detected by the first and second light emitters 3050, 3052. Suitable optical proximity thresholds (e.g., a detected optical property, e.g., wavelength or intensity, reflected from skin or oral tissue of the patient) can be established. The optical property is useful to assess whether the light emitters 3044 of the light array 3042 (and, by proxy, the mouthpiece 3010) is or are properly disposed in the patient's mouth for administering a treatment session. The apparatus 3000 can be configured to initiate irradiation of the oral tissue (i.e., begin the treatment session) when the first and second light emitters 3050, 3052 detect the light reflection from the oral soft tissue.

Each of the first and second light emitters 3050, 3052, can be coupled to the flexible circuit board 3046 in any suitable location. In one or more embodiments, the first and second light emitters 3050, 3052 are disposed on the flexible circuit board 3046 such that each of the first and second light emitters 3050, 3052 is positioned close to the patient's gum when the mouthpiece 3010 is disposed within the patient's mouth. As shown in FIGS. 126-127, the first and second light emitters 3050, 3052 are each disposed offset from parallel rows and/or columns of light emitters 3044 of the light array 3042. Each of the first and second light emitters 3050, 3052 can be disposed between a bottom row (in the orientation of the apparatus 3000 shown in FIG. 127) of light emitters 3044 and the bite tray 3012 of the mouthpiece 3010. In one or more embodiments, the first and second light emitters 3050, 3052 are substantially level (e.g., protruding or regressed by no more than 2-3 mm) with grooves 3032, 3033 defined by an outer face of the mouthpiece 3010. In use, when light is emitted from the light emitters 3044 of the light array 3042, the first and second light emitters 3050, 3052 each measure and/or produce a signal associated with an amount of light reflected by the patient's oral tissue (e.g., the gum). As such, the first and second light emitters 3050, 3052 act as a sensor configured to detect light. Each of the first and second light emitters 3050, 3052 comprises a diode (not shown in FIGS. 126-127) configured to produce an electrical current associated with the amount of light detected or received. The first and second light emitters 3050, 3052, and their diodes, can be configured to send a signal associated with the measured light to a controller (e.g., in an extra-oral housing or bill) of the light therapy apparatus 3000. In one or more embodiments, the diodes of the first and second light emitters 3050, 3052 is configured to measure reflected light at about 855 nm.

At least a portion of the first and second light emitters 3050, 3052 can be embedded in the flanges 3022, 3024 of the mouthpiece 3010, for example, in a similar manner as disclosed herein with respect to the light array 2542. The first and second light emitters 3050, 3052 are spaced apart on the flexible circuit board 3046. In one or more embodiments, the first and second light emitters 3050, 3052 are disposed at opposing locations with respect to the flexible circuit board 3046, as shown in FIG. 127, such that the first light emitter 3050 is beneath the portion of the light array 3042 embedded in the first flange 3022 and the second light emitter 3052 is beneath the portion of the light array 3042 embedded in the second flange 3024. In this manner, the apparatus 3000 is configured to detect reflected light bilaterally.

As disclosed herein, the first and second light emitters 3050, 3052 are configured to be disposed in close proximity to the patient's oral (e.g., gum) tissue when the mouthpiece 3010 is disposed within the patient's mouth in preparation for treatment. The apparatus 3000 is configured to irradiate light only after light having a predetermined optical property, e.g., wavelength or intensity has been measured. Stated another way, the apparatus 3000, and the controller more specifically, is configured to turn on the light emitters 3044 for a treatment session only after the predetermined optical property of reflected light has been measured. The measured amount of reflected light is registered by the controller, which is configured to execute an algorithm to register the amount of reflected light, when (1) the predetermined optical property of reflected light is detected by each of the first and second light emitters 3050, 3052 (i.e., bilaterally), and/or (2) the predetermined amount of reflected light is detected for a predetermined duration (e.g., for at least 2 seconds).

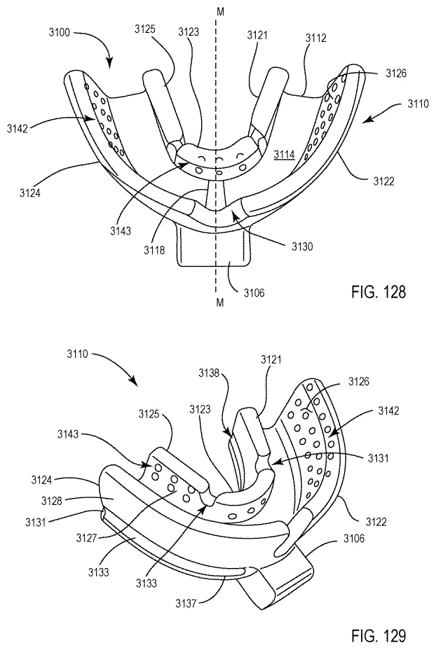

An intra-oral housing 3110 (also referred to herein as a “mouthpiece” of a light therapy apparatus 3100 according to an embodiment is illustrated in FIGS. 128-132. In one or more embodiments, at least one or more portions of the mouthpiece 3110 are constructed from a substantially transparent material (e.g., silicone) such that one or more components embedded within the mouthpiece 3110 are visible through the mouthpiece 3110. Thus, for purposes of illustration, portions of the mouthpiece 3110, including portions of the first flange 3122 and the second flange 3124 are shown as being transparent in FIGS. 128-132 to show portions of the electronics assembly 3140 and the structure disposed therein. The light therapy apparatus 3100 can be included in a light therapy system that is similar in many respects to the light therapy system described herein with respect to FIGS. 84-113. The light therapy apparatus 3100 is configured to irradiate light in any suitable manner described herein, including, for example, to irradiate the alveolar mucosa and/or tooth root area of the patient. Similarly stated, the light therapy apparatus 3100 is configured to administer light therapy to a patient's teeth and/or oral mucosa. More specifically, the light therapy apparatus 3100 is configured to administer light to the patient's teeth and/or oral mucosa sufficient to accelerate orthodontic movement of the patient's teeth and to reduce the overall treatment time for the patient when undergoing orthodontic treatment. The light therapy apparatus 3100 can be the same as or similar in many respects to, or comprise components the same as or similar in many respects to, the intra-oral apparatuses described herein, including, for example, apparatus 2100, apparatus 2500, and apparatus 3000.

The light therapy apparatus 3100 (and any light therapy apparatus described herein) is configured to be useful in combination with traditional orthodontic treatment with an orthodontic appliance, such as brackets and wires, or aligners. Furthermore, in one or more embodiments, any light therapy apparatus shown and described herein can be useful with any suitable orthodontic appliance, including, but not limited to, substantially transparent aligners. Such aligners are orthodontic appliances configured to move a patient's teeth and generally include one or more substantially transparent, removable trays that fit over one or more of the patient's teeth. Each tray of the set of trays is worn by the patient in a predetermined sequence or order, and sometimes for a specified amount or period of time. In particular instances, such aligners or trays generally conform to a patient's teeth but is slightly out of alignment with the starting (e.g., initial) tooth configuration. In this manner, the aligners or trays can exert a force on the teeth. In one or more embodiments, the orthodontic appliance (e.g., brackets and wires or an aligner) is configured to exert a force on one or more of the patient's teeth in an amount (or magnitude) effective to move the patient's teeth towards alignment. In one or more embodiments, the orthodontic appliance is configured to exert a force on one or more of the patient's teeth in an amount (or magnitude) effective to move the patient's teeth, for example, in one or more embodiments, for alignment. Similarly stated, the orthodontic appliance is configured to exert a force to minimize or close a gap or space between the patient's teeth. For example, the orthodontic appliance can be configured to exert an orthodontic force, a less-than-orthodontic force, or a heavy force, as described in detail herein, or a combination thereof, on one or more of the patient's teeth in an amount (or magnitude) effective for tooth movement (e.g., towards alignment or to minimize or close a gap between the patient's teeth).

The intra-oral housing 3110 of the light therapy apparatus 3100 is configured to be disposed in an oral cavity (e.g., in the mouth, not shown in FIGS. 128-132) of a patient. The intra-oral housing 3110 can be configured to be electronically and/or physically coupled to an external controller. In one or more embodiments, for example, the intra-oral housing 3110 is configured to be coupled to an extra-oral housing (or “bill,” not shown in FIGS. 128-132) that is disposed externally to the patient's mouth when the intra-oral housing (or mouthpiece) 3110 is disposed within the patient's mouth. The extra-oral housing can be similar in many respects, or identical, to the extra-oral housing 2560 described herein with respect to FIGS. 84-113, and thus is not described in detail with respect to light therapy apparatus 3100. In other embodiments, the intra-oral housing 3110 is configured to be coupled to the controller via one or more wire or cable connectors (not shown in FIGS. 128-132).

The light therapy apparatus 3100 is configured to be useful for light therapy with the upper jaw and/or the lower jaw of the patient. In other words, the light therapy apparatus 3100 can be configured to administer light therapy with respect to the patient's upper jaw when the apparatus is in an upright position (e.g., as shown in FIG. 131), and can be configured to administer light therapy with respect to the patient's lower jaw when the apparatus is in an inverted position (e.g., not shown in FIGS. 128-132). As such, the mouthpiece 3110 is configured to be disposed within the patient's oral cavity with respect to each of the upper and lower jaws of the patient. Similarly stated, the mouthpiece 3110 is configured to matingly adapt to both the upper jaw and the lower jaw, as described herein, thus eliminating the need for a separate mouthpiece for each jaw. It should be noted that although the light therapy apparatus 3100 generally, and the mouthpiece 3110 specifically, can be described as being in the upright position when configured to be oriented with respect to the upper jaw and in the inverted position when configured to be oriented with respect to the lower jaw, in other embodiments, the light therapy apparatus 3100 and the mouthpiece 3110 are in the upright position when configured to be oriented with respect to the lower jaw of the patient, and in the inverted position when configured to be oriented with respect to the upper jaw of the patient.

The mouthpiece 3110 can be similar in one or more respects, and comprise components similar in one or more respects to the intra-oral housings described herein, including, for example, the intra-oral housings or mouthpieces described herein with reference to FIGS. 35-37, 52-59, 65-72, 84-113 and 126-127. The mouthpiece 3110 comprises a bite tray 3112, flanges 3122, 3124, 3121, 3123, 3125, light arrays 3142, 3143 (see, e.g., FIG. 131). The mouthpiece 3110 can optionally comprise a support plate (not shown in FIGS. 128-132) similar or identical to the support plate 2554 of mouthpiece 2510. The bite tray 3112 is configured to receive at least a portion of the patient's teeth of the upper and/or lower jaw. As such, the bite tray 3112 is generally U-shaped, as shown in FIG. 131. The bite tray 3112 is configured to facilitate proper positioning of the mouthpiece 3110 within the patient's mouth. The bite tray 3112 generally comprises the lower portion of the mouthpiece 3110. The bite tray 3112 comprises a bite pad 3114 with an inner perimeter (or side wall) 3115 and an outer perimeter (or side wall) 3117 (see FIG. 131).

Flanges 3122, 3124, 3121, 3123, 3125, described in more detail herein, generally define an upper portion of the mouthpiece 3110. Outer flanges 3122, 3124 are coupled to the outer perimeter 3117 of the bite pad 3114. Inner flanges 3121, 3123, 3125 are coupled to the inner perimeter 3115 of the bite pad 3114. The flanges 3122, 3124, 3121, 3123, 3125 of the mouthpiece 3110 each extend and/or protrude from the bite pad 3114 in a first direction. As such, when the mouthpiece 3110 is disposed within the patient's mouth, the bite tray 3112 is positioned within the mouth such that the bite pad 3114 is adjacent the occlusal surface of one or more teeth, the outer flanges 3122, 3124 are disposed between the one or more teeth and buccal tissue, and the inner flanges 3121, 3123, 3125 are disposed between the one or more teeth and the tongue and/or palate. Similarly stated, the bite tray 3112 is configured such that when the mouthpiece 3110 is disposed within a mouth, a least a portion of one or more teeth are positioned between the outer flanges 3122, 3124 and the inner flanges 3121, 3123, 3125.

The bite tray 3112 can be similar in many respects, or identical, to the bite tray 2512 described with respect to FIGS. 84-113, and thus is not described in detail with respect to mouthpiece 3110. For example, the bite pad 3114 the bite tray 3112 can have any thickness suitable for receiving a bite force thereon, including a constant or spatially varied thickness as described with respect to bite pad 2514. In another example, the bite tray 3112 (and/or bite pad 3114) can be of any suitable dimensions, including those described herein with respect to bite tray 2512 (and/or bite pad 2514, respectively), and can be constructed of any suitable material, including those described herein with respect to bite tray 2512 (and/or bite pad 2514).

As shown in FIG. 128, an upper surface of the bite pad 3114 comprises a ridge 3118. The ridge 3118 is disposed along a midline M of the mouthpiece 3110 and is elevated with respect to the upper surface of the bite tray 3112 and/or bite pad 3114. The ridge 3118 can extend between the inner perimeter 3115 of the bite pad 3114 and the outer perimeter 3117 of the bite pad 3114. The ridge 3118 facilitates positioning of the mouthpiece 3110 within the patient's oral cavity. For example, the mouthpiece 3110 is configured to be positioned within the patient's oral cavity such that the ridge 3118 is disposed between the patient's front central incisors (on either the upper jaw or the lower jaw). Proprioception of the patient related to the teeth and periodontium can produce sensory feedback to the patient regarding the position of the ridge 3118 of the mouthpiece 3110.

In this manner, the ridge 3118 facilitates centering of the mouthpiece 3110 within the oral cavity, thus promoting symmetry of a light therapy treatment on the alveolar mucosa, or other oral tissue, on both sides of the patient's mouth. In other words, in order to promote the symmetrical administration of light therapy to the root area, the mouthpiece 3110 can be positioned with the midline M of the mouthpiece 3110 seated along the sagittal plane or within (i.e., plus or minus) 5 degrees of the sagittal plane, and the ridge 3118 can facilitate such positioning in use. The ridge 3118 can have any suitable shape, including, for example, the shape of an inverted V, such that the point of the V can be disposed between the patient's front central incisors.

As described herein, the upper portion of the mouthpiece 3110 comprises outer and inner flanges. The outer flanges comprise an outer first (or left) flange 3122 and an outer second (or right) flange 3124. The inner flanges comprise an inner first (or left) flange 3121, an inner second (or middle) flange 3123, and an inner third (or right) flange 3125. Although the outer and inner flanges are shown and described herein as including two and three flanges, respectively, in other embodiments, a mouthpiece can comprise a different number of outer and/or inner flanges.

The upper portion (i.e., the flanges 3122, 3124, 3121, 3123, 3125) of the mouthpiece 3110 is disposed transversely with respect to the bite plate 3114. The flanges 3122, 3124, 3121, 3123, 3125 are configured to be disposed, when the mouthpiece 3110 is disposed within the patient's mouth, such that the bite tray 3112 is adjacent an occlusal surface of the patient's teeth, adjacent a portion of a side of the patient's teeth and/or adjacent the alveolar mucosa. For example, the outer flanges 3122, 3124 can be disposed adjacent a portion of a buccal side of the patient's teeth and/or adjacent a buccal side of the alveolar mucosa. In this manner, a light array 3142 enclosed in the outer flanges 3122, 3124 (also referred to herein as “first light array”), as described in more detail herein, is useful for administering light to the patient's teeth and/or alveolar mucosa (e.g., towards the buccal side of the patient's teeth and/or alveolar mucosa). In another example, the inner flanges 3121, 3123, 3125 can be disposed adjacent a portion of a lingual or palatial side of the patient's teeth and/or adjacent a lingual or palatial side of the alveolar mucosa. In this manner, a light array 3143 enclosed in the inner flanges 3121, 3123, 3125 (also referred to herein as “second light array”), as described in more detail herein, is useful for administering light to the patient's teeth and/or alveolar mucosa (e.g., towards the lingual or palatial side of the patient's teeth and/or alveolar mucosa).

The outer flanges 3122, 3124 collectively contain the first light array 3142, and are each configured to be disposed between the buccal tissue and the alveolar mucosa. Thus, in use, the outer flanges 3122 and 3124 displace oral soft tissue to maintain the desired position of the light array 3142 relative to the anatomy of the patient. More specifically, the outer flanges 3122, 3124 are each configured to displace buccal tissue away from the patient's alveolar mucosa. In one or more embodiments, an inner face 3126 of the outer flanges 3122, 3124 can be spaced apart from the patient's alveolar tissue when the mouthpiece 3110 is disposed within the patient's mouth and the outer flanges 3122, 3124 are displacing the buccal tissue. In one or more embodiments, at least a portion of the inner face 3126 of the outer flanges 3122, 3124 can contact the patient's alveolar tissue when the mouthpiece 3110 is disposed within the patient's mouth and the outer flanges 3122, 3124 are displacing the buccal tissue.

The inner flanges 3121, 3123, 3125 collectively contain the second light array 3143, and are each configured to be disposed between the patient's tongue and/or palate and the alveolar mucosa. Thus, in use, the inner flanges 3121, 3123, 3125 can displace oral soft tissue to maintain the desired position of the second light array 3143 relative to the anatomy of the patient. More specifically, the inner flanges 3121, 3123, 3125 are each configured to displace lingual tissue away from, or otherwise prevent the lingual tissue from contacting, the patient's alveolar mucosa. In one or more embodiments, an inner face 3127 of the inner flanges 3121, 3123, 3125 (see FIG. 129) can be spaced apart from the patient's alveolar tissue when the mouthpiece 3110 is disposed within the patient's mouth and the inner flanges 3121, 3123, 3125 are displacing the lingual tissue. In one or more embodiments, at least a portion of the inner face 3127 of the inner flanges 3121, 3123, 3125 can contact the patient's alveolar tissue when the mouthpiece 3110 is disposed within the patient's mouth and the inner flanges 3121, 3123, 3125 are displacing the lingual tissue.

The flanges 3122, 3124, 3121, 3123, 3125 of the mouthpiece 3110 are configured to be flexible and/or deformable. Similarly stated, the flanges 3122, 3124, 3121, 3123, 3125 are constructed from a material and have geometrical dimensions and/or configurations to provide the desired flexibility, as described herein. Moreover, each of the outer first and second flanges 3122, 3124 and the inner first, second and third flanges 3121, 3123, 3125 is independently deflectable, movable and/or deformable with respect to the mouthpiece 3110 and/or each other. In this manner, the mouthpiece 3110 can be easily disposed within the oral cavity for a variety of different patients having a variety of different anatomical structures, as described herein.

For example, the mouthpiece 3110 includes particular geometric features (e.g., stress concentration risers, areas having a desired bending moment of inertia, etc.) to produce the desired flexibility, deformability and durability in connection with the material(s) from which the mouthpiece 3110 is constructed. As shown, the mouthpiece 3110 defines a notch 3130 and grooves 3132, 3133 configured to permit, or otherwise increase the ability of, the outer flanges 3122, 3124 to deflect inwardly towards the teeth, gums, jaw, or the like (as described herein with respect to mouthpiece 312510 and FIG. 90). As shown in FIGS. 128-131, the mouthpiece 3110 defines the notch 3130 aligned with the midline M of the mouthpiece and between upper portions of the outer first flange 3122 and the outer second flange 3124. The notch 3130 is configured to permit the independent and/or inward deflection of each of the outer first flange 3122 and the outer second flange 3124, for example, in response to pressure from the patient's lip or inner cheek. In particular, the outer flanges 3122, 3124 are each configured to deflect inwardly with respect to the bite pad 3114. Similarly stated, when the mouthpiece 3110 is outside of the mouth in an undeformed state (i.e., a first configuration), the outer first flange 3122 and the outer second flange 3124 are each approximately perpendicular (e.g., at about 90 degrees) to the bite pad 3114. When the mouthpiece 3110 is disposed inside the mouth, the upper portion of the mouthpiece 3110 and/or the outer flanges 3122, 3124 are sufficiently flexible such that an angle formed between each outer flange 3122, 3124 and the bite pad 3114 (an “outer flange angle”) is acute. This “tipping in” allows the outer flanges 3122, 3124 to conform to the interior surfaces of the mouth, thereby promoting the desired alignment of the light array 3142 relative to the bone and/or teeth.

The configuration of the notch 3130, including its shape and dimensions, can be similar in many respects, or identical, to notch 2530 described herein (e.g., with respect to FIG. 90), and thus is not described in detail with respect to mouthpiece 3110. For example, in one or more embodiments, an edge of each outer flange 3122, 3124 that forms a respective side of the notch 3130 tapers towards the point of a V-shape of the notch, which point can be substantially aligned (e.g., plus or minus about 5 degrees) with an upper edge of the grooves 3132, 3133. In another example, in other embodiments, the portion of the mouthpiece 3110 that defines a lower boundary of the notch 3130 can be in any suitable location relative to the grooves 3132, 3133 (e.g., either above or below the grooves).

The mouthpiece 3110 defines at least one groove 3132, 3133 defined by a lower outer (or front) surface of each of the outer first and second flanges 3122, 3124. For example, the mouthpiece 3110 comprises the first groove 3132 and the second groove 3133, each defined by the outer or front surface 3128 of the mouthpiece 3110. The grooves 3132, 3133 can each be similar in many respects, or identical, to grooves 2532, 2533 described with respect to mouthpiece 2510 and FIGS. 84-113. The grooves 3132, 3133 are each disposed at a height between the bite pad 3114 and a lower edge of a flexible circuit board 3146 (see, e.g., FIGS. 129 and 131) of the mouthpiece 3110. Stated another way, the grooves 3132, 3133 can be defined by a base portion of each of the outer first and second flanges 3122, 3124. The grooves 3132, 3133 each extend about the outer surface 3128 of the mouthpiece 3110 between the posterior end portion of the mouthpiece 3110 and an anterior end portion of the mouthpiece 3110, such that a first end 3134, 3135 of each groove 3132, 3133, respectively, is at or proximate to the posterior end portion of the mouthpiece 3110 and a second end 3136, 3137 of each groove 3132, 3133, respectively, is at or proximate to the anterior end of the mouthpiece 3110.

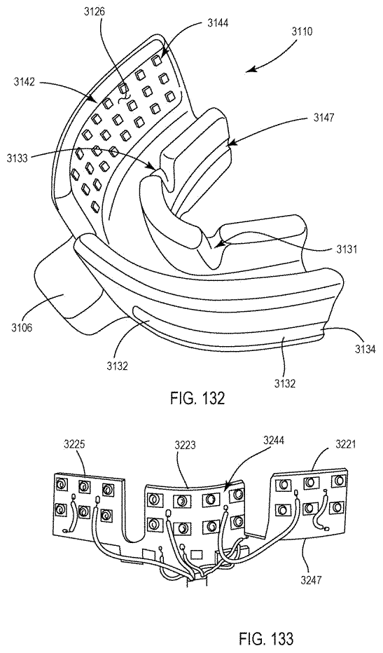

As shown in FIGS. 129 and 132, the second ends 3136, 3137 of the grooves 3132, 3133 can be spaced apart. In other words, the second ends 3136, 3137 of the grooves 3132, 3133 do not necessarily meet at the anterior end of the mouthpiece 3110. Similarly stated, the grooves 3132, 3133 are noncontiguous and/or do not share a common boundary. For example, the second ends 3136, 3137 of the grooves 3132, 3133 can be spaced apart by a width of a bridge 3106 extending from the front of the mouthpiece 3110. In another example, the second ends 3136, 3137 of the grooves 3132, 3133 can be spaced apart by a distance at least as great as a width of the notch 3130. The grooves 3132, 3133 can have any suitable shape, including, for example, that of a semi-circle or U-shape. The grooves 3132, 3133 produce a hinge-like structure (i.e., a “living hinge”) about which the flanges 3122, 3124 can rotate, bend and/or deflect. In this manner, the grooves 3132, 3123 and the notch 3130 collectively permit the flanges 3122, 3124 to deflect inwardly, for example, in response to pressure from the patient's lip or inner cheek.

As such, the grooves 3132, 3133 and the notch 3130 collectively facilitate the transition of the mouthpiece 3110 between a first configuration and a second configuration. When the mouthpiece 3110 is in the first configuration, the angle formed between each flange 3122, 3124 and the bite pad 3114 (the “outer flange angle”) has a first value. When the mouthpiece 3110 is in the second configuration, the outer flange angle has a second value that is different from the first value. In particular, the mouthpiece 3110 can be moved to the second configuration when disposed within the patient's mouth. In one or more embodiments, the second value is less than the first value (i.e., the outer flanges 3122, 3124 “tip” inwardly toward the bite plate 3112 when the mouthpiece 2510 is inserted into the mouth). In one or more embodiments, the outer flange angle is approximately 90 degrees when the mouthpiece is in the first configuration and is acute when the mouthpiece is in the second configuration. In one or more embodiments, the outer flange angle is about 80 degrees (e.g., the outer flanges 3122, 3124 tip inward toward the bite plate 3112 by about 10 degrees) when the mouthpiece is in the second configuration. In other embodiments, the outer flange angle is between about 75 degrees and about 80 degrees (e.g., the outer flanges 3122, 3124 tip inward toward the bite plate 3112 by between about 10 degrees and 15 degrees). In yet other embodiments, the outer flange angle is approximately 85 degrees, 75 degrees, 70 degrees, or 65 degrees (e.g., the outer flanges 3122, 3124 tip inward toward the bite plate by about 5 degrees, 15 degrees, about 20 degrees and about 25 degrees, respectively) when the mouthpiece is in the second configuration.

As shown, the mouthpiece 3110 also defines notches 3131, 3131′ configured to permit, or otherwise increase the ability of, the inner flanges 3121, 3123, 3125 to deflect inwardly towards the bite plate 3112 (or outwardly towards the teeth, gums, jaw, or the like in a direction opposite to the inward deflection described herein with respect to outer flanges 3122, 3124). As shown in FIGS. 129-131, the mouthpiece 3110 defines a first notch 3131 between upper portions of the first and second inner flanges 3121, 3123 of the mouthpiece, and a second notch 3131′ between upper portions of the second and third inner flanges 3123, 3125 of the mouthpiece. Each notch 3131, 3131′ of the inner flanges 3121, 3123, 3125 can be positioned on the mouthpiece 3110 equidistant from the midline M. An edge of the first and second inner flanges 3121, 3123 forms a respective side of the first notch 3131. An edge of the second inner flange 3123 (different from the edge forming a side of the first notch 3131) and an edge of the third inner flange 3123 form respective sides of the second notch 3131′.

The first and second notches 3131, 3131′ are configured to permit the independent and/or inward deflection of each of the inner first, second and third flanges 3121, 3123, 3125, for example, in response to pressure from the patient's tongue. In particular, the inner flanges 3121, 3123, 3125 are each configured to deflect inwardly with respect to the bite pad 3114. Similarly stated, when the mouthpiece 3110 is outside of the mouth in its first configuration, in an undeformed state, the inner first, second and third flanges 3121, 3123, 3125 are each approximately perpendicular to the bite pad 3114. When the mouthpiece 3110 is disposed inside the mouth, the upper portion of the mouthpiece 3110 and/or the inner flanges 3121, 3123, 3125 are sufficiently flexible such that an angle formed between each inner flange 3121, 3123, 3125 and the bite pad 3114 (an “inner flange angle”) is acute. This “tipping in” allows the inner flanges 3122, 3124 to conform to the interior surfaces of the mouth, thereby promoting the desired alignment of the second light array 3143 relative to the bone and/or teeth.

The first and second notches 3131, 3131′ of the inner flanges 3121, 3123, 3125 can have any suitable shape and/or dimension. As shown in FIGS. 129 and 132, an upper portion of the notches 3131, 3131′ can be U-shaped. In one or more embodiments, a lower portion of the notches 3131, 3131′ can be a vertically elongate opening or slit extended from the U-shaped upper portion of each notch 3131, 3131′. In one or more embodiments, the lower portion of each notch 3131, 3131′ can comprise an unbroken portion of material disposed adjacent a lower end of the U-shaped portion. The lower portion of each notch 3131, 3131′ has a thickness less than a thickness of an adjacent inner flange (e.g., flange 3121, 3123, 3125) and is configured to separate, break, and/or otherwise tear, when a deflection force is applied to one or more of the inner flanges 3121, 3123, 3125. In this manner, the lower portion of each notch 3131, 3131′ can be configured, to separate, break, and/or otherwise tear, when the mouthpiece is in its second configuration within the patient's mouth to form a vertically elongate opening or slit extended from the lower end of the U-shaped portion of the notch.

The mouthpiece 3110 can define a groove 3138 defined by a lower outer (or rear) surface of the inner flanges 3121, 3123, 3125. For example, as shown in FIG. 131, the mouthpiece 3110 comprises the groove 3138 defined by an outer or rear surface of the mouthpiece 3110. The groove 3138 can be similar in many respects, or identical, to grooves 3132, 3133. The groove 3138 is disposed at a height between the bite pad 3114 and a lower edge of a flexible circuit board 3147 (see, e.g., FIG. 131) of the mouthpiece 3110. Stated another way, the groove 3138 can be collectively defined by a base portion of each of the first, second and third inner flanges 3121, 3123, 3125, such that such that ends of the groove 3138 are each at or proximate to the posterior end portion of the mouthpiece 3110.

The groove 3138 can have any suitable shape, including, for example, that of a semi-circle or U-shape. The groove 3138 produces a hinge-like structure (i.e., a “living hinge”) about which the inner flanges 3121, 3123, 3125 can rotate, bend and/or deflect. In this manner, the groove 3128 and the first and second notches 3131, 3131′ collectively permit the inner flanges 3121, 3123, 3125 to deflect inwardly with respect to the bite plate (or outwardly with respect to the tongue and/or palate), for example, in response to pressure from the patient's tongue.

As such, the groove 3138 and the first and second notches 3131, 3131′ collectively facilitate the transition of the mouthpiece 3110 between its first configuration and its second configuration. When the mouthpiece 3110 is in the first configuration, the angle formed between each inner flange 3121, 3123, 3125 and the bite pad 3114 (the “inner flange angle”) has a first value. When the mouthpiece 3110 is in the second configuration, the inner flange angle has a second value that is different from the first value. In particular, the mouthpiece 3110 can be moved to the second configuration when disposed within the patient's mouth. In one or more embodiments, the second value is less than the first value (i.e., the inner flanges 3121, 3123, 3125 3124 “tip” inwardly towards the bite plate when the mouthpiece 2510 is inserted into the mouth). In one or more embodiments, the inner flange angle is approximately 90 degrees when the mouthpiece is in the first configuration and is acute when the mouthpiece is in the second configuration. In one or more embodiments, the inner flange angle is about 80 degrees (e.g., the inner flanges 3121, 3123, 3125 tip inward towards the bite plate by about 10 degrees) when the mouthpiece is in the second configuration. In other embodiments, the inner flange angle is between about 75 degrees and about 80 degrees (e.g., the inner flanges 3121, 3123, 3125 tip inward toward the bite plate by between about 10 degrees and 15 degrees). In yet other embodiments, the inner flange angle is approximately 85 degrees, 75 degrees, 70 degrees, or 65 degrees (e.g., the inner flanges 3121, 3123, 3125 tip inward towards the bite plate by about 5 degrees, 15 degrees, about 20 degrees and about 25 degrees, respectively) when the mouthpiece is in the second configuration.

The flexibility of the mouthpiece 3110, and of the flanges 3122, 3124, 3121, 3123, 3125 in particular, provides significant advantages. For example, in contrast to mouthpieces constructed of a hard plastic and/or with a permanent set (or shape), the current arrangement allows for easier insertion and better conformance to the oral tissue of the patient. The flexibility of the mouthpiece 3110 also accommodates variation in patient anatomy (whether between two different patients or for the same patient as that patient's anatomy changes over time). For example, some patients have a pronounced overbite and might need more or less than a 10 degree inward deflection (or “tip-in”). In such instances, the mouthpiece 3110 can conform to the internal structure and/or anatomy within the patient's mouth. As another example, as the orthodontia for a patient works over time, the patient's dental anatomy will change. Accordingly, the mouthpiece 3110 can conform to the internal structure and/or anatomy within the patient's mouth to accommodate such change without requiring new mouthpiece moldings or the like. Finally, the flexible design of the mouthpiece 3110 provides greater comfort for the patient than would be provided by mouthpieces constructed of a hard plastic.

Additionally, the flexible nature of the mouthpiece 3110 and/or the flanges 3122, 3124, 3121, 3123, 3125 provides manufacturing benefits. In particular, fabrication and/or molding of a mouthpiece having an acute angle between the bite surface and the side surface of the flange (i.e., the internal angle of the flange or the “flange angle”) can be difficult. The design of the mouthpiece 3110, however, allows for the molding and/or fabrication to be performed with a flange angle of approximately ninety degrees (or greater), while allowing for an in-use flange angle that is acute (e.g., when the mouthpiece 3110 is in the second configuration, as described herein).

The mouthpiece 3110 of the light therapy apparatus 3100 comprises an electronics assembly 3140, generally shown in FIG. 131. The electronics assembly 3140 can be similar in many respects, or identical to, the electronics assembly 2540 of mouthpiece 2510 described herein (e.g., with respect to FIGS. 93-95). As shown, a first portion of the electronics assembly 3140 of the mouthpiece 3110 is disposed primarily in the flanges 3122, 3124. The first portion of the electronics assembly 3140 comprises a light array 3142 and a flexible circuit board 3146. A second portion of the electronics assembly 3140 is disposed primarily in the inner flanges 3121, 3123, 3125. The second portion of the electronics assembly 3140 comprises a light array 3143 and a flexible circuit board 3147. The light arrays 3142, 3143 each comprise one or more light emitters 3144, 3145, such as a plurality of LEDs. The light emitters 3144, 3145 are electrically and/or physically coupled to the flexible circuit boards 3146, 3147, respectively (only a portion of the flexible circuit board 3147 is shown in FIG. 109). The flexible circuit boards 3146, 3147, respectively, electrically couple the light emitters 3144, 3145, respectively, to electronic circuitry outside of the mouthpiece 3110 (e.g., in an extra-oral housing or via electrical connectors to an external controller, not shown). In this manner, the light emitters 3144, 3145, respectively, can receive power and/or a signal to produce the desired light, as described herein.

Referring to FIG. 131, the light emitters 3144 of the first portion of the electronics assembly 3140 are disposed on a first, palatial (or lingual) side of the flexible circuit board 3146 of the first portion of the electronics assembly. The light emitters 3145 of the second portion of the electronics assembly 3140 are disposed on a buccal side of the flexible circuit board 3147 of the second portion of the electronics assembly. In this manner, the light emitters 3144, 3145 are configured to emit light toward a patient's teeth and/or adjacent oral tissue when the mouthpiece 3110 is disposed within the patient's mouth. Stated another way, the light emitters 3144 are configured to emit light towards the anterior root area of an upper and/or lower jaw and/or the buccal alveolar soft tissue and the light emitters 3145 are configured to emit light towards a posterior root area of an upper and/or lower jaw and/or the lingual alveolar soft tissue.

The light emitters 3144, 3145 can be configured to emit light at any suitable intensity, wavelength and/or frequency described herein. For example, in one or more embodiments, the light emitters 3144, 3145 can be configured to emit light in the infrared or near infrared wavelength range. For example, in one or more embodiments, the light emitters 3144, 3145 are configured to emit light at a wavelength of about 850 nm. In one or more embodiments, the light emitters 3144, 3145 are configured to emit light at a wavelength of 850 nm±5 nm. The light emitters 3144, 3145 can be configured to emit light sufficient deliver light energy to the patient's bone to facilitate and/or perform any of the methods described herein. The light emitters 3144, 3145 can be configured to emit light at less than 150 mW/cm².

The light emitters 3144, 3145 can be disposed on the flexible circuit boards 3146, 3147, respectively, and/or within the flanges 3122, 3124 and the inner flanges 3121, 3123, 3125, respectively, in any suitable configuration, including any configuration described herein. For example, in one or more embodiments, the light emitters 3144, 3145 are LEDs coupled to the flexible circuit boards 3146, 3147 in two or more parallel rows and/or columns. In one or more embodiments, the light emitters 3144 are coupled to the flexible circuit board 3146 of the first portion of the electronics assembly 3140 in three parallel rows, and the light emitters 3145 are coupled to the flexible circuit board of the second portion of the electronics assembly in two parallel rows.

The light array 3142 of the first portion of the electronics assembly 3140 can comprise about 54 light emitters 3144, or LEDs, with about 27 light emitters embedded in the first flange 3122 and about 27 light emitters 3144 embedded in the second flange 3124. The 27 light emitters 3144 can be arranged in any suitable configuration, including for example in nine evenly spaced columns with three spaced apart light emitters, or LEDs, per column. The light array 3143 of the second portion of the electronics assembly 3140 can comprise about 18 or 20 light emitters 3145, or LEDS, with about 6 light emitters embedded in each of the first and third inner flanges 3121, 3125, and 6 light emitters embedded in the second (or middle) panel 3123. The 6 light emitters 3145 can be arranged in any suitable configuration, including, for example, in three evenly spaced columns with two spaced apart light emitters, or LEDs, per column. The flexible circuit boards 3146, 3147 and light emitters 3144, 3145 can have any suitable dimensions for being coupled to, or embedded in, the outer flanges 3122, 3124 and the inner flanges 3121, 3123, 3125, respectively, of the mouthpiece 3110.

Although the mouthpiece 3110 has been shown as including 6 light emitters 3145 embedded in the inner flanges 3121, 3123, 3125, in other embodiments, the mouthpiece can comprise a different number of light emitters in the inner flanges, and each inner flange can comprise a different number of light emitters than another inner flange. For example, as shown in FIG. 133, a flexible circuit board 3247 comprises first and third portions 3221, 3225, each having six (6) light emitters in two evenly spaced rows configured to be disposed in each of a first and third inner flange (not shown in FIG. 133) and a middle portion 3223 having eight (8) light emitters 3244 in two evenly spaced rows and configured to be disposed in a second (or middle) inner flange (not shown in FIG. 133). As also shown in FIG. 133, the flexible circuit board 3247 is configured such that its first portion 3221 is spaced apart from its second portion 3223, and its second portion is spaced apart from its third portion 3225, which facilitates the deflection (“tipping out” or “tipping in”) of the inner flanges.

Returning to FIGS. 128-132, although the light emitters 3144 are shown as being evenly spaced within their respective flanges 3122, 3124 or inner flanges 3121, 3123, 3125, in other embodiments, the light emitters can be unevenly spaced within their respective flanges 3122, 3124 and/or inner flanges 3121, 3123, 3125. For example, in one or more embodiments, a mouthpiece can comprise a series of light emitters that are spaced apart by a first amount near the anterior portion of the mouthpiece and a second, different amount near the posterior portion of the mouthpiece. Light emitters of the series of light emitters can be disposed on or in, e.g., embedded in, the mouthpiece.

The mouthpiece 3110 can be constructed of any suitable material, including, for example, any material described herein with respect to mouthpiece 2510, and thus such material is not described in detail with respect to mouthpiece 3110. For example, the mouthpiece 3110 can be constructed of an elastomeric material (e.g., a soft silicone). In another example, the mouthpiece 3110 can be fabricated from medical-grade injection-molded, highly flexible and very low durometer silicone. In another example, the silicone and/or portions of the mouthpiece 3110 are substantially transparent, such that one or more components embedded within the silicone are visible through the silicone. Moreover, in this manner, the mouthpiece 3110 can provide suitable optical properties for allowing the light produced and/or conveyed by the light emitters 3144, 3145 to pass through the mouthpiece 3110 to the desired target tissue. In one or more embodiments, the mouthpiece 3110, the flanges 3122, 3124, and/or the inner flanges 3121, 3123, 3125 can comprise one or more components configured to filter, focus and/or otherwise act upon the light produced by the light emitters 3144, 3145. In other embodiments, the mouthpiece 3110 can comprise air gaps between the light emitters 3144, 3145 and the surface of the flanges 3122, 3124 and inner flanges 3121, 3123, 3125, respectively, to facilitate focusing of the light. As shown in FIGS. 128-132, however, the mouthpiece 3110 is constructed such that the light emitters 3144, 3145 are fully encapsulated or embedded within the molded silicone such that no space or air gap exists between the silicone material and the first and second portions of the electronics assembly 3140. Similarly stated, the mouthpiece 3110 is devoid of an air gap between the light emitters 3144, 3145 and the material of the mouthpiece 3110, thus no air gap lensing is needed to produce the desired optical properties of the light produced by the light emitters 3144, 3145.

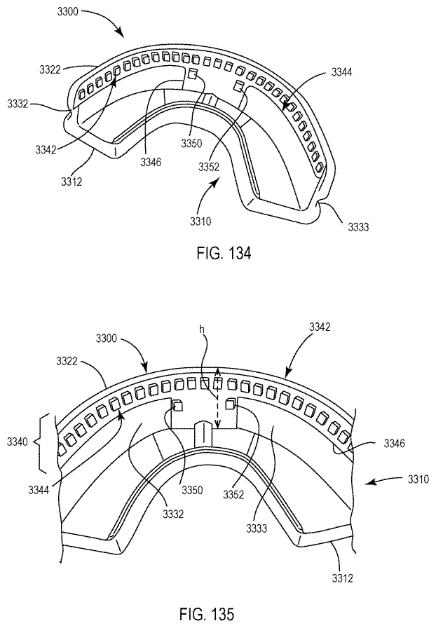

As shown in FIGS. 134-135, in one or more embodiments, the intraoral light therapy apparatus 3300 provided herein comprises a single row of light emitters such as LEDs 3342. The single row of LEDs, in one or more embodiments provides about 50 milliwatt (mW) to about 200 mW of light power per cm², for example about 50 mW to about 150 mW per cm². In a further embodiment, the light emitters of one of the therapy apparatuses provided herein that comprises a single row of light emitters provides about 100 mW per cm² of light power.

As shown in FIGS. 134-135, in one or more embodiments the light therapy apparatus having a single row of light emitters 3300 (i.e., columns of light emitters with a single light emitter, e.g., LED per column) comprises an intra-oral housing or mouthpiece 3310 configured to be disposed within a mouth of a patient. The mouthpiece 3310 comprises a bite tray 3312, a flange 3322 transversely coupled to the bite tray, an electronics assembly 3340 including a light array 3342 and a flexible circuit board 3346. In one or more embodiments, the light therapy apparatus 3300 comprises an extra-oral housing (not shown) configured to be disposed outside the mouth of the patient.

The light emitters are coupled to the electronics assembly such that zones of the light emitters are individually addressable. One or more light emitters (e.g., one or more LEDs) can constitute a zone of light emitters. In one or more embodiments, from about 1 to about 20, from about 1 to about 15, from about 1 to about 10, or from about 1 to about 9, or from about 1 to about 8, or from about 1 to about 7, or from about 1 to about 6, or from about 1 to about 5, or from about 1 to about 4 light emitters are included in a zone of light emitters. It will be understood by one of skill in the art that the number of light emitters harnessed during light therapy will vary depending on patient presentation and ultimate treatment protocol.

The outer flange (also referred to as “buccal flange”) of a single row light emitter light therapy apparatus provided herein can be characterized by a height “h”. In one or more embodiments, the height “h” is about 1 cm, about 1.2 cm, about 1.5 cm, about 1.8 cm, about 2.1 cm, about 2.4 cm, about 2.7 cm or about 3 cm, including all values, ranges and subranges in between. As illustrated in FIGS. 134-135, in one or more embodiments, the outer flange 3322 can be relatively shorter, or have a lower profile, than the flange 3022 of the mouthpiece 3010.

In one or more embodiments, the outer flange 3322 can be relatively more rigid than the flange 3022 and/or 3024, while still providing a comfortable feel and fit to a user/patient. In one or more embodiments, the rigidity of the flange is achieved by not including a notch in the flange. Specifically, in one or more embodiments, the mouthpiece 3310 can comprise a single outer flange 3322 formed between the ends of the mouthpiece 3010 with a continuous top edge, such that there is no notch present, unlike the gap depicted in other embodiments, e.g., in the mouthpiece 3010 between the outer flange 3022 and the outer flange 3024.

As also illustrated in FIGS. 134-135, and as disclosed herein, in one or more embodiments, the light array 3342 comprises a single row of light emitters such as LEDs. The reduced number of light emitters in the mouthpiece 3310 provides for reduced power consumption when compared to the mouthpiece 3010. In one or more embodiments, the number of light emitters in the light array 3342 is about 15, about 18, about 21, about 24, about 27, about 30, about 33, about 36, about 39, about 42, about 45, including all values and subranges in between. In one or more embodiments, the number of light emitters in the light array 3342 is greater than the number of light emitters in a single row of the light array 3042. In this manner, in one or more embodiments, the mouthpiece 3310 has relatively fewer light emitters than the mouthpiece 3010, but has a relatively greater density of light emitters, such that the optical power density during use can be similar, or even greater, with the mouthpiece 3310 than with the mouthpiece 3010. Generally, the power density of any of the light emitters disclosed herein can be about 30 mW/cm², about 40 mW/cm², about 50 mW/cm², about 60 mW/cm², about 80 mW/cm², about 100 mW/cm², about 120 mW/cm², about 140 mW/cm², about 150 mW/cm², including all values and sub-ranges in between.

In one or more embodiments, the outer flange 3312 is sized such that the row of light emitters of the light array 3342 is positioned above the upper gum line of the patient during use. In some embodiments, the outer flange 3312 is sized such that the row of light emitters of the light array 3342 is positioned at the gum line of the patient during use, between the gum line and the most apical portion of the tooth root during use, or at the most apical portion of the tooth root during use.

In one or more embodiments, at least one or more portions of the mouthpiece 3310 are constructed from one or more substantially transparent materials (e.g., silicone) such that one or more components embedded within the mouthpiece 3310 are visible through the mouthpiece 3310. Thus, for purposes of illustration, portions of the mouthpiece 3310, including portions of the outer flange 3322, the bite tray 3312 is shown as being transparent in FIGS. 112 and 113 to show portions of the electronics assembly 3340 and the structure disposed therein. In one or more embodiments, multiple layers of material are used to increase the rigidity of the outer flange 3322.

The light therapy apparatus 3300 is configured to determine whether the mouthpiece 3310 is disposed within the patient's mouth (i.e., in a manner suitable for a treatment session, as described herein). In this manner, the light therapy apparatus 3300 can be configured to only irradiate light for the treatment session when the apparatus 3300 has determined that the mouthpiece 3310 is disposed in the patient's mouth. The mouthpiece 3310 comprises a first light emitter 3350 and a second light emitter 3352, each of which is configured and positioned to detect or measure an amount of light reflected by a portion of the patient's oral tissue. For example, the first and second light emitters 3350, 3352 can be configured to detect light emitted from the light emitters 3344 of the light array 3342 and reflected by the patient's oral soft tissue. In one or more embodiments, the light emitters 3344 can be configured to emit light, such as in a blinking or pulsing manner, and can be addressable individually, or in groups. The light emitters 3344 can be configured to blink or pulse at a predetermined rate. At least some light emitted from the pulsing or blinking light emitters 3344 towards the oral soft tissue of the patient's mouth is reflected to the mouthpiece 3310 and is thereby detected by the first and second light emitters 3350, 3352. Suitable optical proximity thresholds (e.g., related to an optical property, e.g., wavelength or intensity, of light reflected from skin of the patient) can be established and used in assessing whether the light emitters 3344 of the light array 3342 (and, by proxy, the mouthpiece 3310) are properly disposed in the patient's mouth for administering a treatment session. The apparatus 3300 can be configured to initiate irradiation of the oral tissue (i.e., begin the treatment session) when the first and second light emitters 3350, 3352 detect the light reflection from the oral soft tissue.

Each of the first and second light emitters 3350, 3352, can be coupled to the flexible circuit board 3346 in any suitable location. In one or more embodiments, the first and second light emitters 3350, 3352 are disposed on the flexible circuit board 3346 such that each of the first and second light detectors 3350, 3352 is positioned close to the patient's gum when the mouthpiece 3310 is disposed within the patient's mouth. As shown in FIGS. 134-135, the first and second light emitters 3350, 3352 are each disposed offset from a single parallel row of light emitters 3344 of the light array 3342. Each of the first and second light detectors 3350, 3352 can be disposed between the single row (in the orientation of the apparatus 3300 shown in FIG. 135) of light emitters 3344 and the bite tray 3012 of the mouthpiece 3010. In one or more embodiments, the first and second light emitters 3350, 3352 are substantially level with grooves 3332, 3333 (e.g., protruding or regressed by no more than 2-3 mm) defined by an outer face of the mouthpiece 3310. In use, when light is emitted from the light emitters 3344 of the light array 3342, the first and second light emitters 3350, 3352 each measure and/or produce a signal associated with an amount of light reflected by the patient's oral tissue (e.g., the gum). As such, the first and second light emitters 3350, 3352 act as a sensor configured to detect reflected light. Each of the first and second light emitters 3350, 3352 in one or more embodiments, comprises a diode (not shown in FIGS. 134-135) configured to produce an electrical current associated with the amount of light detected or received. The first and second light emitters 3350, 3352, and their one or more diodes, can be configured to send a signal associated with the measured reflected light to a controller (e.g., in an extra-oral housing or bill) of the light therapy apparatus 3300. In one or more embodiments, the one or more diodes of the first and second light emitters 3350, 3352 are configured to measure reflected light at about 815 nm, about 820 nm, about 825 nm, about 830 nm, about 835 nm, about 840 nm, about 845 nm, about 850 nm, about 855 nm, about 860 nm, about 865 nm, about 870 nm, about 875 nm, about 880 nm, about 885 nm, about 890 nm, about 895 nm, about 900 nm, including all values and subranges in between.

At least a portion of the first and second light emitters 3350, 3352 can be fully or at least partially embedded in the outer flange 3322 of the mouthpiece 3310, for example, in a similar manner as disclosed herein with respect to the light array 2542. The first and second light emitters 3350, 3352 are spaced apart on the flexible circuit board 3346. In one or more embodiments, the first and second light emitters 3350, 3352 are disposed at opposing locations with respect to the flexible circuit board 3346, as shown in FIG. 135. In this manner, the apparatus 3300 is configured to detect reflected light bilaterally.

As disclosed herein, the first and second light emitters 3350, 3352 are configured to be disposed in close proximity (e.g., based on measurement of reflected light in response to irradiance) to the patient's oral (e.g., gum) tissue when the mouthpiece 3310 is disposed within the patient's mouth in preparation for treatment. The apparatus 3300 is configured to irradiate light only after a predetermined property, e.g., wavelength or intensity, of reflected light has been measured. Stated another way, the apparatus 3300, and the controller more specifically, can be configured to turn on the light emitters 3344 for a treatment session only after the predetermined property of reflected light has been measured. The measured amount of reflected light is registered by the controller, which is configured to execute an algorithm to register the property of reflected light, when (1) the predetermined property of reflected light is detected by each of the first and second light emitters 3350, 3352 (i.e., bilaterally), and/or (2) the predetermined property of reflected light is detected for a predetermined duration (e.g., for at least 2 seconds).

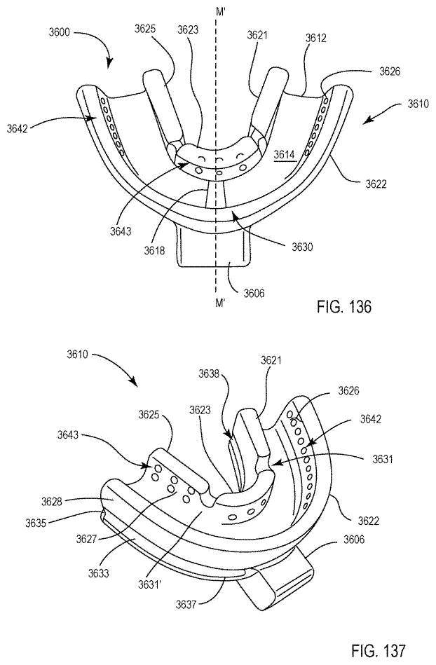

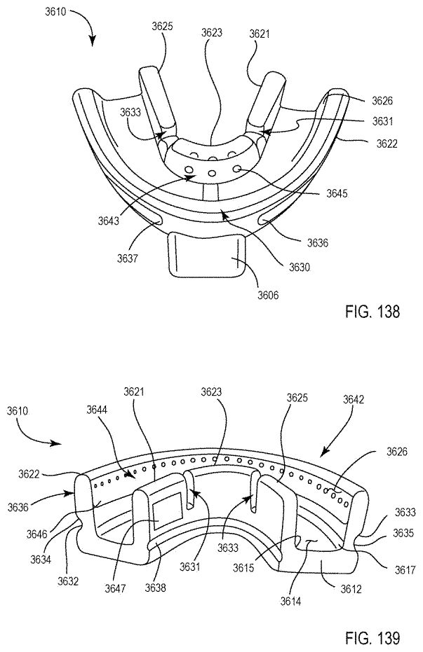

An intra-oral housing 3610 (also referred to herein as a “mouthpiece”) of a light therapy apparatus 3600 according to an embodiment is illustrated in FIGS. 136-140. In one or more embodiments, at least one or more portions of the mouthpiece 3610 are constructed from a substantially transparent material (e.g., silicone) such that one or more components embedded within the mouthpiece 3610 are visible through the mouthpiece 3610. Thus, for purposes of illustration, portions of the mouthpiece 3610, including portions of the outer flange 3622, are shown as being transparent in FIGS. 136-140 to show portions of the electronics assembly 3640 and the structure disposed therein. The light therapy apparatus 3600 can be included in a light therapy system that is similar in many respects to the light therapy system described herein with respect to FIGS. 84-113. The light therapy apparatus 3600 is configured to irradiate light in any suitable manner described herein, including, for example, to irradiate the alveolar mucosa and/or tooth root area of the patient. Similarly stated, the light therapy apparatus 3600 is configured to administer light therapy to a patient's teeth and/or oral mucosa. More specifically, the light therapy apparatus 3600 is configured to administer light to the patient's teeth and/or oral mucosa sufficient to accelerate orthodontic movement of the patient's teeth and to reduce the overall treatment time for the patient when undergoing orthodontic treatment. In this manner, aspects of bone health can be improved by usage of the light therapy apparatus 3600, alone or in combination with use of an orthodontic appliance, when compared with use of an orthodontic appliance in the absence of light therapy.

The light therapy apparatus 3600 can be the same as or similar in many respects to, or comprise components the same as or similar in many respects to, the intra-oral apparatuses described herein, including, for example, apparatus 2100, apparatus 2500, apparatus 3000, and apparatus 3100. However, as described herein, the light therapy apparatus 3600 comprises a single row of light emitters in contrast to previously described embodiments. In addition, the light therapy apparatus 3600 has an increased rigidity in one or more embodiments, due to a flange with a continuous top edge, i.e., a notch is not present.

The light therapy apparatus 3600 (and any light therapy apparatus described herein) is configured to be useful in combination with traditional orthodontic treatment with an orthodontic appliance, such as brackets and wires, or aligners. Furthermore, in one or more embodiments, any light therapy apparatus shown and described herein can be used with any suitable orthodontic appliance, including, but not limited to, substantially transparent aligners. Such aligners are orthodontic appliances and are described herein.

The intra-oral housing 3610 of the light therapy apparatus 3600 is configured to be disposed in an oral cavity (e.g., in the mouth, not shown in FIGS. 128-132) of a patient. The intra-oral housing 3610 can be configured to be electronically and/or physically coupled to an external controller. In one or more embodiments, for example, the intra-oral housing 3610 is configured to be coupled to an extra-oral housing (or “bill,” not shown in FIGS. 136-140) that is disposed externally to the patient's mouth when the intra-oral housing (or mouthpiece) 3610 is disposed within the patient's mouth. The extra-oral housing can be similar in many respects, or identical, to the extra-oral housing 2560 described herein with respect to FIGS. 84-113, and thus is not described in detail with respect to light therapy apparatus 3600. In other embodiments, the intra-oral housing 3610 is configured to be coupled to the controller via one or more wire or cable connectors (not shown in FIGS. 136-140).

The light therapy apparatus 3600 is configured to be useful for light therapy with the upper jaw and/or the lower jaw of the patient. In other words, the light therapy apparatus 3600 can be configured to administer light therapy with respect to the patient's upper jaw when the apparatus is in an upright position (e.g., as shown in FIG. 139), and can be configured to administer light therapy with respect to the patient's lower jaw when the apparatus is in an inverted position (not shown in FIGS. 136-140). As such, the mouthpiece 3610 is configured to be disposed within the patient's oral cavity with respect to each of the upper and lower jaws of the patient. Similarly stated, in one or more embodiments, the mouthpiece 3610 is configured to matingly adapt to both the upper jaw and the lower jaw, as described herein, thus eliminating the need for a separate mouthpiece for each jaw. It should be noted that although the light therapy apparatus 3600 generally, and the mouthpiece 3610 specifically, can be described as being in the upright position when configured to be oriented with respect to the upper jaw and in the inverted position when configured to be oriented with respect to the lower jaw, in other embodiments, the light therapy apparatus 3600 and the mouthpiece 3610 are in the upright position when configured to be oriented with respect to the lower jaw of the patient, and in the inverted position when configured to be oriented with respect to the upper jaw of the patient.

The mouthpiece 3610 can be similar in one or more respects, and comprise components similar in one or more respects to the intra-oral housings described herein, including, for example, the intra-oral housings or mouthpieces described herein with reference to FIGS. 35-37, 52-59, 65-72, 84-113, 126-127, and 134-135. The mouthpiece 3610 comprises a bite tray 3612, flanges 3622, 3621, 3623, 3625, light arrays 3642, 3643 (see, e.g., FIG. 139). The mouthpiece 3610 can optionally comprise a support plate (not shown in FIGS. 136-140) similar or identical to the support plate 2554 of mouthpiece 2510. The bite tray 3612 is configured to receive at least a portion of the patient's teeth of the upper and/or lower jaw. As such, the bite tray 3612 is generally U-shaped, as shown in FIG. 139. The bite tray 3612 is configured to facilitate proper positioning of the mouthpiece 3610 within the patient's mouth. The bite tray 3612 generally comprises the lower portion of the mouthpiece 3610. The bite tray 3612 comprises a bite pad 3614 with an inner perimeter (or side wall) 3615 and an outer perimeter (or side wall) 3617 (see FIG. 139).

Flanges 3622, 3621, 3623, 3625, described in more detail herein, generally define an upper portion of the mouthpiece 3610. The outer flange 3622 is coupled to the outer perimeter 3617 of the bite pad 3614. Inner flanges 3621, 3623, 3625 are coupled to the inner perimeter 3615 of the bite pad 3614. The flanges 3622, 3621, 3623, 3625 of the mouthpiece 3610 each extend and/or protrude from the bite pad 3614 in a first direction. As such, when the mouthpiece 3610 is disposed within the patient's mouth, the bite tray 3612 is positioned within the mouth such that the bite pad 3614 is adjacent the occlusal surface of one or more teeth, the outer flange 3622 is disposed between the one or more teeth and buccal tissue, and the inner flanges 3621, 3623, 3625 are disposed between the one or more teeth and the tongue and/or palate. Similarly stated, the bite tray 3612 is configured such that when the mouthpiece 3610 is disposed within a mouth, a least a portion of one or more teeth are positioned between the outer flange 3622 and the inner flanges 3621, 3623, 3625.

The bite tray 3612 can be similar in many respects, or identical, to the bite tray 2512 described with respect to FIGS. 84-113, and thus is not described in detail with respect to mouthpiece 3610. For example, the bite pad 3614 the bite tray 3612 can have any thickness suitable for receiving a bite force thereon, including a constant or spatially varied thickness as described with respect to bite pad 2514. In another example, the bite tray 3612 (and/or bite pad 3614) can be of any suitable dimensions, including those described herein with respect to bite tray 2512 (and/or bite pad 2514, respectively), and can be constructed of any suitable material, including those described herein with respect to bite tray 2512 (and/or bite pad 2514).

As shown in FIG. 136, in one or more embodiments, an upper surface of the bite pad 3614 comprises a ridge 3618. The ridge 3618 is disposed along a midline M′ of the mouthpiece 3610 and is elevated with respect to the upper surface of the bite tray 3612 and/or bite pad 3614. The ridge 3618 can extend between the inner perimeter 3615 of the bite pad 3614 and the outer perimeter 3617 of the bite pad 3614. The ridge 3618 facilitates positioning of the mouthpiece 3610 within the patient's oral cavity. For example, the mouthpiece 3610 is configured to be positioned within the patient's oral cavity such that the ridge 3618 is disposed between the patient's front central incisors (on either the upper jaw or the lower jaw). Proprioception of the patient related to the teeth and periodontium can produce sensory feedback to the patient regarding the position of the ridge 3618 of the mouthpiece 3610.

In this manner, the ridge 3618 facilitates centering of the mouthpiece 3610 within the oral cavity, thus promoting symmetry of a light therapy treatment on the alveolar mucosa, or other oral tissue, on both sides of the patient's mouth. In other words, in order to promote the symmetrical administration of light therapy to the root area, the mouthpiece 3610 can be positioned with the midline M′ of the mouthpiece 3610 seated along the sagittal plane or within (i.e., plus or minus) 5 degrees of the sagittal plane, and the ridge 3618 can facilitate such positioning in use. The ridge 3618 can have any suitable shape, including, for example, the shape of an inverted V, such that the point of the V can be disposed between the patient's front central incisors.

As described herein, the upper portion of the mouthpiece 3610 comprises an outer flange 3622 and inner flanges. The inner flanges comprise an inner first (or left) flange 3621, an inner second (or middle) flange 3623, and an inner third (or right) flange 3625. Although the outer flange and the inner flanges are shown and described herein as including one and three flanges, respectively, in other embodiments, a mouthpiece can comprise a different number of outer and/or inner flanges.

As illustrated in FIGS. 136-140, in one or more embodiments, the flange 3622 can be relatively shorter, or have a lower profile, than the flange 3122 of the mouthpiece 3110. In one or more embodiments, the flange 3622 can be relatively more rigid than the flange 3122 and/or 3124, while still providing a comfortable feel and fit to a user/patient. As also illustrated in FIGS. 136-140, in one or more embodiments, the mouthpiece 3610 can comprise a single outer flange 3622 formed between the ends of the mouthpiece 3610, such that there is no notch present, unlike the notch 3130 in the mouthpiece 3110 between the flange 3122 and the flange 3124.

As also illustrated in FIGS. 136-140, in one or more embodiments, the light array 3642 can include a single row of light emitters such as LEDs, and is differentiated in this manner from the light array 3142, which can comprise multiple rows and columns of light emitters. The reduced number of light emitters in the mouthpiece 3610 provides for reduced power consumption when compared to the mouthpiece 3110. In one or more embodiments, the number of light emitters in the light array 3642 is about 15, about 18, about 21, about 24, about 27, about 30, about 33, about 36, about 39, about 42, about 45, including all values and subranges in between. In one or more embodiments, the number of light emitters in the light array 3642 is greater than the number of light emitters in a single row of the light array 3142. In this manner, in one or more embodiments, the mouthpiece 3610 has relatively fewer light emitters than the mouthpiece 3110, but has a relatively greater density of light emitters, such that the optical power density during use can be similar, or even greater, with the mouthpiece 3610 than with the mouthpiece 3110. In one or more embodiments, the flange 3612 is sized such that the row of light emitters of the light array 3642 is positioned above the upper gum line of the patient during use.

The upper portion (i.e., the flanges 3622, 3622, 3623, 3625) of the mouthpiece 3610 is disposed transversely with respect to the bite plate 3614. The flanges 3622, 3621, 3623, 3625 are configured to be disposed, when the mouthpiece 3610 is disposed within the patient's mouth, such that the bite tray 3612 is adjacent an occlusal surface of the patient's teeth, adjacent a portion of a side of the patient's teeth and/or adjacent the alveolar mucosa. For example, the outer flange 3622 can be disposed adjacent a portion of a buccal side of the patient's teeth and/or adjacent a buccal side of the alveolar mucosa. In this manner, a light array 3642 enclosed in the outer flange 3622 (also referred to herein as “first light array”), as described in more detail herein, can be useful for administering light to the patient's teeth and/or alveolar mucosa (e.g., towards the buccal side of the patient's teeth and/or alveolar mucosa). In another example, the inner flanges 3621, 3623, 3625 can be disposed adjacent a portion of a lingual or palatial side of the patient's teeth and/or adjacent a lingual or palatial side of the alveolar mucosa. In this manner, a light array 3643 enclosed in the inner flanges 3621, 3623, 3625 (also referred to herein as “second light array”), as described in more detail herein, can be useful for administering light to the patient's teeth and/or alveolar mucosa (e.g., towards the lingual or palatial side of the patient's teeth and/or alveolar mucosa).

The outer flange 3622 contains the first light array (single row) 3642, and is configured to be disposed between the buccal tissue and the alveolar mucosa. Thus, in use, the outer flange 3622 displaces oral soft tissue to maintain the desired position of the light array 3642 relative to the anatomy of the patient. More specifically, the outer flange 3622 is configured to displace buccal tissue away from the patient's alveolar mucosa. In one or more embodiments, an inner face 3626 of the outer flange 3622 can be spaced apart from the patient's alveolar tissue when the mouthpiece 3610 is disposed within the patient's mouth and the outer flange 3622 is displacing the buccal tissue. In one or more embodiments, at least a portion of the inner face 3626 of the outer flange 3622 can contact the patient's alveolar tissue when the mouthpiece 3610 is disposed within the patient's mouth and the outer flange 3622 is displacing the buccal tissue.

In some embodiment, the inner flange or inner flanges contain a second light array. For example, as shown in FIG. 137, the inner flanges 3621, 3623, 3625 collectively contain the second light array 3643, and are each configured to be disposed between the patient's tongue and/or palate and the alveolar mucosa. Thus, in use, the inner flanges 3621, 3623, 3625 can displace oral soft tissue to maintain the desired position of the second light array 3643 relative to the anatomy of the patient. More specifically, the inner flanges 3621, 3623, 3625 are each configured to displace lingual tissue away from, or otherwise prevent the lingual tissue from contacting, the patient's alveolar mucosa. In one or more embodiments, an inner face 3627 of the inner flanges 3621, 3623, 3625 (see FIG. 137) can be spaced apart from the patient's alveolar tissue when the mouthpiece 3610 is disposed within the patient's mouth and the inner flanges 3621, 3623, 3625 are displacing the lingual tissue. In one or more embodiments, at least a portion of the inner face 3627 of the inner flanges 3621, 3623, 3625 can contact the patient's alveolar tissue when the mouthpiece 3610 is disposed within the patient's mouth and the inner flanges 3621, 3623, 3625 are displacing the lingual tissue.

The outer flange 3622 and inner flanges 3621, 3623, 3625 of the mouthpiece 3610 are configured in one or more embodiments to be flexible and/or deformable. Similarly stated, the outer flange 3622 and the inner flanges 3621, 3623, 3625 are constructed from a material and have geometrical dimensions and/or configurations to provide the desired flexibility, as described herein. Moreover, each of the inner first, second and third flanges 3621, 3623, 3625 is independently deflectable, movable and/or deformable with respect to the mouthpiece 3610 and/or each other. In this manner, the mouthpiece 3610 can be easily disposed within the oral cavity for a variety of different patients having a variety of different anatomical structures, as described herein.

For example, the mouthpiece 3610 comprises particular geometric features (e.g., stress concentration risers, areas having a desired bending moment of inertia, etc.) to produce the desired flexibility, deformability and durability in connection with the material(s) from which the mouthpiece 3610 is constructed. As shown, the mouthpiece 3610 defines grooves 3632, 3633 configured to permit, or otherwise increase the ability of, the outer flange 3622 to deflect inwardly towards the teeth, gums, jaw, or the like (as described herein with respect to mouthpiece 2510 and FIG. 90) (and, in some embodiments including other mouthpiece embodiments described herein, additionally or alternatively, outwardly). In particular, the outer flange 3622 is configured to deflect inwardly with respect to the bite pad 3614. Similarly stated, when the mouthpiece 3610 is outside of the mouth in an undeformed state (i.e., a first configuration), the outer flange 3622 is approximately perpendicular (e.g., about 90 degrees) to the bite pad 3614. When the mouthpiece 3610 is disposed inside the mouth, the upper portion of the mouthpiece 3610 and/or the outer flange 3622 are sufficiently flexible such that an angle formed between the outer flange 3622 and the bite pad 3614 (an “outer flange angle”) is acute. This “tipping in” allows the outer flange 3622 to conform to the interior surfaces of the mouth, thereby promoting the desired alignment of the light array 3642 relative to the bone and/or teeth.

The mouthpiece 3610 defines at least one groove 3632, 3633 defined by a lower outer (or front) surface of the outer flange 3622. For example, the mouthpiece 3610 comprises the first groove 3632 and the second groove 3633, each defined by the outer or front surface 3628 of the mouthpiece 3610. The grooves 3632, 3633 can each be similar in many respects, or identical, to grooves 2532, 2533 described with respect to mouthpiece 2510 and FIGS. 84-113, and to grooves 3132, 3133 described with respect to mouthpiece 3110 and FIGS. 128-132. The grooves 3632, 3633 are each disposed at a height between the bite pad 3614 and a lower edge of a flexible circuit board 3646 (see, e.g., FIGS. 137 and 139) of the mouthpiece 3610. Stated another way, the grooves 3632, 3633 can be defined by a base portion of the outer flange 3622. The grooves 3632, 3633 each extend about the outer surface 3628 of the mouthpiece 3610 between the posterior end portion of the mouthpiece 3610 and an anterior end portion of the mouthpiece 3610, such that a first end 3634, 3635 of each groove 3632, 3633, respectively, is at or proximate to the posterior end portion of the mouthpiece 3610 and a second end 3636, 3637 of each groove 3632, 3633, respectively, is at or proximate to the anterior end of the mouthpiece 3610.

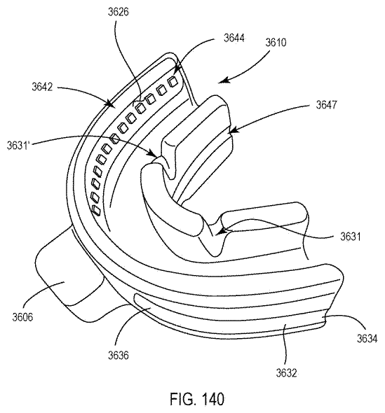

As shown in FIGS. 137 and 140, the second ends 3636, 3637 of the grooves 3632, 3633 can be spaced apart. In other words, the second ends 3636, 3637 of the grooves 3632, 3633 do not necessarily meet at the anterior end of the mouthpiece 3610. Similarly stated, the grooves 3632, 3633 are noncontiguous and/or do not share a common boundary. For example, the second ends 3636, 3637 of the grooves 3632, 3633 can be spaced apart by a width of a bridge 3606 extending from the front of the mouthpiece 3610. The grooves 3632, 3633 can have any suitable shape, including, for example, that of a semi-circle or U-shape. The grooves 3632, 3633 produce a hinge-like structure (i.e., a “living hinge”) about which the outer flange 3622 can bend and/or deflect inwardly, for example, in response to pressure from the patient's lip or inner cheek.

As such, the grooves 3632, 3633 collectively facilitate the transition of the mouthpiece 3610 between a first configuration and a second configuration. When the mouthpiece 3610 is in the first configuration, the angle formed between the outer flange 3622 and the bite pad 3614 (the “outer flange angle”) has a first value. When the mouthpiece 3610 is in the second configuration, the outer flange angle has a second value that is different from the first value. In particular, the mouthpiece 3610 can be moved to the second configuration when disposed within the patient's mouth. In one or more embodiments, the second value is less than the first value (i.e., the outer flange 3622 “tips” inwardly toward the bite plate 3612 when the mouthpiece 3610 is inserted into the mouth). In one or more embodiments, the outer flange angle is approximately 90 degrees when the mouthpiece is in the first configuration and is acute when the mouthpiece is in the second configuration. In one or more embodiments, the outer flange angle is about 80 degrees (e.g., the outer flange 3622 tips inward toward the bite plate 3612 by about 10 degrees) when the mouthpiece is in the second configuration. In other embodiments, the outer flange angle is between about 75 degrees and about 80 degrees (e.g., the outer flange 3622 tips inward toward the bite plate 3612 by between about 10 degrees and 15 degrees). In yet other embodiments, the outer flange angle is approximately 85 degrees, 75 degrees, 70 degrees, or 65 degrees (e.g., the outer flange 3622 tips inward toward the bite plate by about 5 degrees, 15 degrees, about 20 degrees and about 25 degrees, respectively) when the mouthpiece is in the second configuration.

As shown, the mouthpiece 3610 also defines notches 3631, 3631′ configured to permit, or otherwise increase the ability of, the inner flanges 3621, 3623, 3625 to deflect inwardly towards the bite plate 3612 (or outwardly towards the teeth, gums, jaw, or the like in a direction opposite to the inward deflection described herein with respect to outer flange 3622). As shown in FIGS. 137-139, the mouthpiece 3610 defines a first notch 3631 between upper portions of the first and second inner flanges 3621, 3623 of the mouthpiece, and a second notch 3631′ between upper portions of the second and third inner flanges 3623, 3625 of the mouthpiece. Each notch 3631, 3631 of the inner flanges 3621, 3623, 3625 can be positioned on the mouthpiece 3610 equidistant from the midline M′. An edge of the first and second inner flanges 3621, 3623 forms a respective side of the first notch 3631. An edge of the second inner flange 3623 (different from the edge forming a side of the first notch 3631) and an edge of the third inner flange 3623 form respective sides of the second notch 3631′.

The first and second notches 3631, 3631′ are configured to allow the independent and/or inward deflection of each of the inner first, second and third flanges 3621, 3623, 3625, for example, in response to pressure from the patient's tongue. In particular, the inner flanges 3621, 3623, 3625 are each configured to deflect inwardly with respect to the bite pad 3614. Similarly stated, when the mouthpiece 3610 is outside of the mouth in its first configuration, in an undeformed state, the inner first, second and third flanges 3621, 3623, 3625 are each approximately perpendicular (e.g., about 90 degrees) to the bite pad 3614. When the mouthpiece 3610 is disposed inside the mouth, the upper portion of the mouthpiece 3610 and/or the inner flanges 3621, 3623, 3625 are sufficiently flexible such that an angle formed between each inner flange 3621, 3623, 3625 and the bite pad 3614 (an “inner flange angle”) is acute. This “tipping in” allows the inner flanges 3621, 3623, 3625 to conform to the interior surfaces of the mouth, thereby promoting the desired alignment of the second light array 3643 relative to the bone and/or teeth.

The first and second notches 3631, 3631′ of the inner flanges 3621, 3623, 3625 can have any suitable shape and/or dimension. As shown in FIGS. 137 and 140, an upper portion of the notches 3631, 3631′ can be U-shaped. In one or more embodiments, a lower portion of the notches 3631, 3631′ can be a vertically elongate opening or slit extended from the U-shaped upper portion of each notch 3631, 3631′. In one or more embodiments, the lower portion of each notch 3631, 3631′ can comprise an unbroken portion of material disposed adjacent a lower end of the U-shaped portion. The lower portion of each notch 3631, 3631′ has a thickness less than a thickness of an adjacent inner flange (e.g., flange 3621, 3623, 3625) and is configured to separate, break, and/or otherwise tear, when a deflection force is applied to one or more of the inner flanges 3621, 3623, 3625. In this manner, the lower portion of each notch 3631, 3631′ can be configured, to separate, break, and/or otherwise tear, when the mouthpiece is in its second configuration within the patient's mouth to form a vertically elongate opening or slit extended from the lower end of the U-shaped portion of the notch.

The mouthpiece 3610 can define a groove 3638 defined by a lower outer (or rear) surface of the inner flanges 3621, 3623, 3625. For example, as shown in FIG. 139, the mouthpiece 3610 comprises the groove 3638 defined by an outer or rear surface of the mouthpiece 3610. The groove 3638 can be similar in many respects, or identical, to grooves 3632, 3631′. The groove 3638 is disposed at a height between the bite pad 3614 and a lower edge of a flexible circuit board 3647 (see, e.g., FIG. 139) of the mouthpiece 3610. Stated another way, the groove 3638 can be collectively defined by a base portion of each of the first, second and third inner flanges 3621, 3623, 3625, such that such that ends of the groove 3638 are each at or proximate to the posterior end portion of the mouthpiece 3610.

The groove 3638 can have any suitable shape, including, for example, that of a semi-circle or U-shape. The groove 3638 produces a hinge-like structure (i.e., a “living hinge”) about which the inner flanges 3621, 3623, 3625 can rotate, bend and/or deflect. In this manner, the groove 3628 and the first and second notches 3631, 3631′ collectively permit the inner flanges 3621, 3623, 3625 to deflect inwardly with respect to the bite plate (or outwardly with respect to the tongue and/or palate), for example, in response to pressure from the patient's tongue.

As such, the groove 3638 and the first and second notches 3631, 3631′ collectively facilitate the transition of the mouthpiece 3610 between its first configuration and its second configuration. When the mouthpiece 3610 is in the first configuration, the angle formed between each inner flange 3621, 3623, 3625 and the bite pad 3614 (the “inner flange angle”) has a first value. When the mouthpiece 3610 is in the second configuration, the inner flange angle has a second value that is different from the first value. In particular, the mouthpiece 3610 can be moved to the second configuration when disposed within the patient's mouth. In one or more embodiments, the second value is less than the first value (i.e., the inner flanges 3621, 3623, 3625 “tip” inwardly towards the bite plate when the mouthpiece 3110 is inserted into the mouth). In one or more embodiments, the inner flange angle is approximately 90 degrees when the mouthpiece is in the first configuration and is acute when the mouthpiece is in the second configuration. In one or more embodiments, the inner flange angle is about 80 degrees (e.g., the inner flanges 3621, 3623, 3625 tip inward towards the bite plate by about 10 degrees) when the mouthpiece is in the second configuration. In other embodiments, the inner flange angle is between about 75 degrees and about 80 degrees (e.g., the inner flanges 3621, 3623, 3625 tip inward toward the bite plate by between about 10 degrees and 15 degrees). In yet other embodiments, the inner flange angle is approximately 85 degrees, 75 degrees, 70 degrees, or 65 degrees (e.g., the inner flanges 3621, 3623, 3625 tip inward towards the bite plate by about 5 degrees, 15 degrees, about 20 degrees and about 25 degrees, respectively) when the mouthpiece is in the second configuration.

For example, some patients have a pronounced overbite and might need more or less than a 10 degree inward deflection (or “tip-in”). In such instances, the mouthpiece 3610 can conform to the internal structure and/or anatomy within the patient's mouth. As another example, as the orthodontia for a patient works over time, the patient's dental anatomy will change. Accordingly, the mouthpiece 3610 can conform to the internal structure and/or anatomy within the patient's mouth to accommodate such change without requiring new mouthpiece moldings or the like.

In one or more embodiments, the design of the mouthpiece 3610 allows for the molding and/or fabrication to be performed with a flange angle of approximately ninety degrees (or greater), while allowing for an in-use flange angle that is acute (e.g., when the mouthpiece 3610 is in the second configuration, as described herein).

The mouthpiece 3610 of the light therapy apparatus 3600 comprises an electronics assembly 3640, generally shown in FIG. 139. The electronics assembly 3640 can be similar in many respects, or identical to, the electronics assembly 2540 of mouthpiece 2510 described herein (e.g., with respect to FIGS. 93-95). As shown, a first portion of the electronics assembly 3640 of the mouthpiece 3610 is disposed primarily in the flange 3622. The first portion of the electronics assembly 3640 comprises a light array 3642 and a flexible circuit board 3646. A second portion of the electronics assembly 3640 is disposed primarily in the inner flanges 3621, 3623, 3625. The second portion of the electronics assembly 3640 comprises a light array 3643 and a flexible circuit board 3647. The light arrays 3642, 3643 each comprise one or more light emitters 3644, 3645, such as a plurality of LEDs. The light emitters 3644, 3645 are electrically and/or physically coupled to the flexible circuit boards 3646, 3647, respectively (only a portion of the flexible circuit board 3647 is shown in FIG. 139). The flexible circuit boards 3646, 3647, respectively, electrically couple the light emitters 3644, 3645, respectively, to electronic circuitry outside of the mouthpiece 3610 (e.g., in an extra-oral housing or via electrical connectors to an external controller, not shown). In this manner, the light emitters 3644, 3645, respectively, can receive power and/or a signal to produce the desired light, as described herein.

Referring to FIG. 139, the light emitters 3644 of the first portion of the electronics assembly 3640 are disposed on a first, palatial (or lingual) side of the flexible circuit board 3646 of the first portion of the electronics assembly. The light emitters 3645 of the second portion of the electronics assembly 3640 are disposed on a buccal side of the flexible circuit board 3647 of the second portion of the electronics assembly. In this manner, the light emitters 3644, 3645 are configured to emit light toward a patient's teeth and/or adjacent oral tissue when the mouthpiece 3610 is disposed within the patient's mouth. Stated another way, the light emitters 3644 are configured to emit light towards the anterior root area of an upper and/or lower jaw and/or the buccal alveolar soft tissue and the light emitters 3645 are configured to emit light towards a posterior root area of an upper and/or lower jaw and/or the lingual alveolar soft tissue.

The light emitters 3644, 3645 can be configured to emit light at any suitable intensity, wavelength and/or frequency described herein. For example, in one or more embodiments, the light emitters 3644, 3645 can be configured to emit light in the infrared or near infrared wavelength range. For example, in one or more embodiments, the light emitters 3644, 3645 are configured to emit light at a wavelength of about 850 nm. In one or more embodiments, the light emitters 3644, 3645 are configured to emit light at a wavelength from about 585 nm to about 665 nm, from about 605 nm to about 645 nm, at about 625 m, from about 815 nm to about 895 nm, from about 835 nm to about 855 nm, including all values and subranges in between. The light emitters 3644, 3645 can be configured to emit light sufficient deliver light energy to the patient's bone to facilitate and/or perform any of the methods described herein. The light emitters 3644, 3645 can be configured to emit light at less than 150 mW/cm².

The light emitters 3644, 3645 can be disposed on the flexible circuit boards 3646, 3647, respectively, and/or within the flange 3622 and the inner flanges 3621, 3623, 3625, respectively, in any suitable configuration, including any configuration described herein. For example, in one or more embodiments, the light emitters 3644, 3645 are LEDs coupled to the flexible circuit boards 3646, 3647 in two or more parallel rows and/or columns. In one or more embodiments, the light emitters 3644 are coupled to the flexible circuit board 3646 of the first portion of the electronics assembly 3640 in a single row, and the light emitters 3645 are coupled to the flexible circuit board of the second portion of the electronics assembly in two parallel rows.

The light array 3643 of the second portion of the electronics assembly 3640 can comprise about 18 or 20 light emitters 3645, or LEDS, with about 6 light emitters embedded in each of the first and third inner flanges 3621, 3625, and 6 light emitters embedded in the second (or middle) panel 3623. The 6 light emitters 3645 can be arranged in any suitable configuration, including, for example, in three evenly spaced columns with two spaced apart light emitters, or LEDs, per column. The flexible circuit boards 3646, 3647 and light emitters 3644, 3645 can have any suitable dimensions for being coupled to, or embedded in, the outer flange 3622 and the inner flanges 3621, 3623, 3625, respectively, of the mouthpiece 3610.

Although the mouthpiece 3610 has been shown as including 6 light emitters 3645 embedded in the inner flanges 3621, 3623, 3625, in other embodiments, the mouthpiece can comprise a different number of light emitters in the inner flanges, and each inner flange can comprise a different number of light emitters than another inner flange, as best illustrated in FIG. 133, described herein.

Returning to FIGS. 136-140, although the light emitters 3644, 3645 are shown as being evenly spaced within the outer flange 3622 or inner flanges 3621, 3623, 3625, in other embodiments, the light emitters can be unevenly spaced within the outer flange 3622 and/or inner flanges 3621, 3623, 3625. For example, in one or more embodiments, a mouthpiece can comprise a series of light emitters that are spaced apart by a first amount near the anterior portion of the mouthpiece and a second, different amount near the posterior portion of the mouthpiece.

The mouthpiece 3610 can be constructed of any suitable material, including, for example, any material described herein with respect to mouthpiece 2510 and/or mouthpiece 3110, and thus such material is not described in detail with respect to mouthpiece 3610. For example, the mouthpiece 3610 can be constructed of an elastomeric material (e.g., a soft silicone). In another example, the mouthpiece 3610 can be fabricated from medical-grade injection-molded, highly flexible and very low durometer silicone. In another example, the silicone and/or portions of the mouthpiece 3610 are substantially transparent, such that one or more components embedded within the silicone are visible through the silicone. Moreover, in this manner, the mouthpiece 3110′ can provide suitable optical properties for allowing the light produced and/or conveyed by the light emitters 3644, 3645 to pass through the mouthpiece 3610 to the desired target tissue. In one or more embodiments, the mouthpiece 3610, the outer flange 3622 and/or the inner flanges 3621, 3623, 3625 can comprise one or more components configured to filter, focus and/or otherwise act upon the light produced by the light emitters 3644, 3645. In other embodiments, the mouthpiece 3610 can comprise air gaps between the light emitters 3644, 3645 and the surface of the outer flange 3622 and inner flanges 3621, 3623, 3625, respectively, to facilitate focusing of the light. As shown in FIGS. 136-140, however, the mouthpiece 3610 is constructed such that the light emitters 3644, 3645 are fully encapsulated or embedded within the molded silicone such that no detectable space or air gap exists between the silicone material and the first and second portions of the electronics assembly 3640. Similarly stated, the mouthpiece 3610 is devoid of an air gap between the light emitters 3644, 3645 and the material of the mouthpiece 3610, thus no air gap lensing is needed to produce the desired optical properties of the light produced by the light emitters 3644, 3645.

In one or more embodiments, any light therapy apparatus disclosed herein (e.g., the light therapy apparatus 3100 and/or the light therapy apparatus 3600) is useful for treating patients with a history of periodontal disease, and/or with reduced periodontal support. In one or more embodiments, the light therapy apparatuses disclosed herein are useful with orthodontic treatment with an orthodontic appliance. In one or more embodiments, the light therapy apparatuses disclosed herein are useful with orthodontic treatment with an orthodontic appliance to retain teeth in their final, desired position, i.e., after treatment. In one or more embodiments, orthodontic treatment with the light therapy apparatuses disclosed herein result in initiating and/or accelerating, relative to no treatment, one or more of orthodontic tooth movement, osteoblast proliferation, collagen deposition, osteoblast activity (e.g., as measured by osteoblast activity markers), osteoclast activity (e.g., as measured by osteoclast activity markers), combinations thereof, and/or the like. In one or more embodiments, orthodontic treatment with the light therapy apparatuses disclosed herein result in initiating and/or accelerating, relative to orthodontic treatment alone, one or more of orthodontic tooth movement, osteoblast proliferation, collagen deposition, osteoblast activity, osteoclast activity, combinations thereof, and/or the like. In one or more embodiments, orthodontic treatment with the light therapy apparatuses disclosed herein in combination with orthodontic treatment result in initiating and/or accelerating, relative to no treatment, one or more of orthodontic tooth movement, osteoblast proliferation, collagen deposition, osteoblast activity, osteoclast activity, combinations thereof, and/or the like. In one or more embodiments, orthodontic treatment with the light therapy apparatuses disclosed herein in combination with orthodontic treatment result in initiating and/or accelerating, relative to orthodontic treatment alone, one or more of orthodontic tooth movement, osteoblast proliferation, collagen deposition, osteoblast activity, osteoclast activity, combinations thereof, and/or the like.

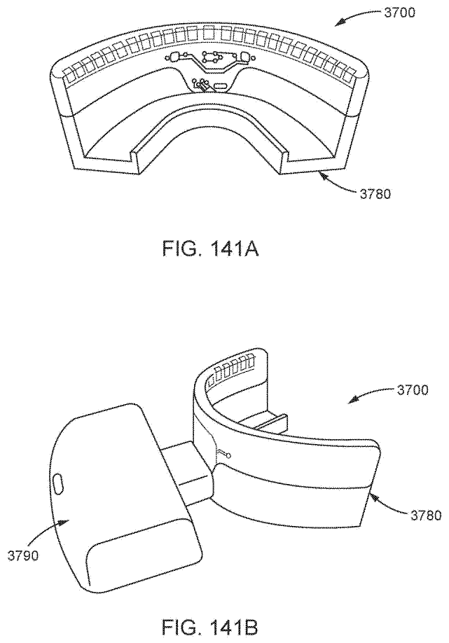

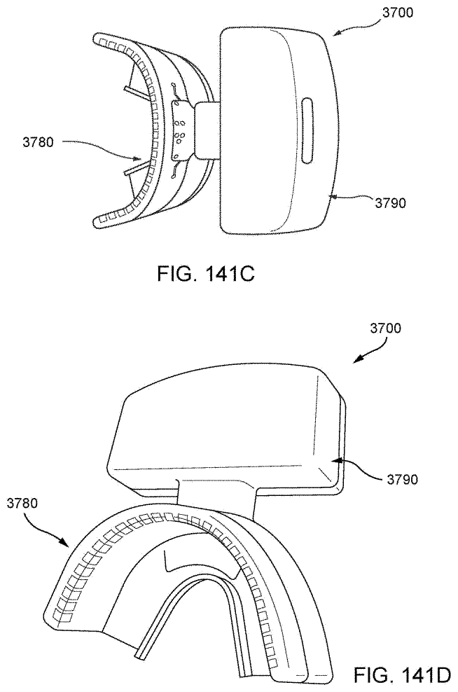

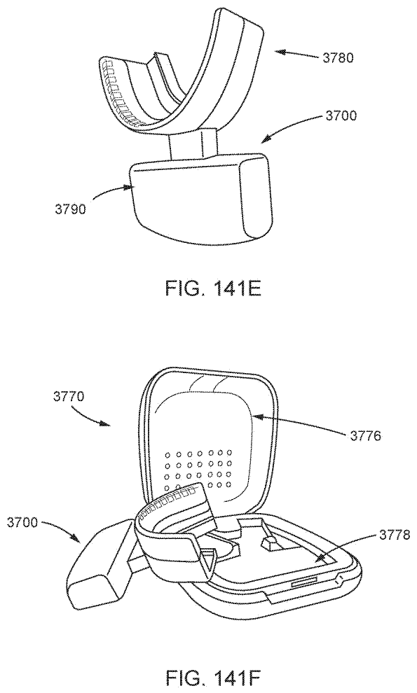

FIGS. 141A-141F illustrate an intra-oral apparatus 3700, according to an embodiment of the invention. The apparatus 3700 can comprise components of other intra-oral apparatuses described herein. The intra-oral apparatus 3700 comprises an intra-oral housing/mouthpiece 3780 configured to be disposed in an oral cavity (e.g., in the mouth) of a patient and an extra-oral housing 3790 (also referred to herein as a “bill”) coupled to the intra-oral housing 3780.

In one or more embodiments, the intra-oral housing 3780 can be the same as or similar in many respects to, or comprise components the same as or similar in many respects to, the intra-oral apparatuses described herein. For example, and as illustrated in FIGS. 141A-141F, the intra-oral housing 3780 can be structurally and/or functionally similar to the mouthpiece 3310. For example, the intra-oral housing 3780 can comprise, similar to the mouthpiece 3310, a bite tray (similar to the bite tray 3312, a single outer flange without a notch (similar to the flange 3322), an electronics assembly (similar to the electronics assembly 3340) including a light array (similar to the light array 3342) and a flexible circuit board (similar to the flexible circuit board 3346). In one or more embodiments, the intra-oral housing 3780 can be structurally and/or functionally similar to the mouthpiece 3610. For example, the intra-oral housing 3780 can comprise, similar to the mouthpiece 3610, a bite tray (similar to the bite tray 3612), an outer flange without a notch (similar to the outer flange 3622), inner flanges (similar to the inner flanges 3621, 3623, 3625), and light arrays (similar to the light arrays 3642, 3643).

FIGS. 141A-141F depict the extra-oral housings 3780 and 3790. In one or more embodiments, the extra-oral housing 3790 can comprise a microprocessor (not shown) (e.g., similar to the microprocessor 2196) configured to store and execute a treatment protocol specific to the nature/capabilities of the intra-oral housing 3780. In some embodiments, the microprocessor of the extra-oral housing 3790 can be configured to execute a treatment protocol that accounts for the single row of light emitters on the intra-oral housing 3780.

Referring to FIG. 141F in particular, the extra-oral housing 3790 can be configured to be disposed on or otherwise coupled to an external station 3770, for example, when the apparatus 3700 is not in use by the patient. The external station 2170 can be, for example a carrying case, charging caddy or station, or the like, or a combination of the foregoing. In one or more embodiments, the external station 3770 can comprise a base 3778 and a lid 3776 (e.g., similar to the base 2178 and the lid 2176, respectively) and defines a cavity configured to hold the intra-oral apparatus 3700.

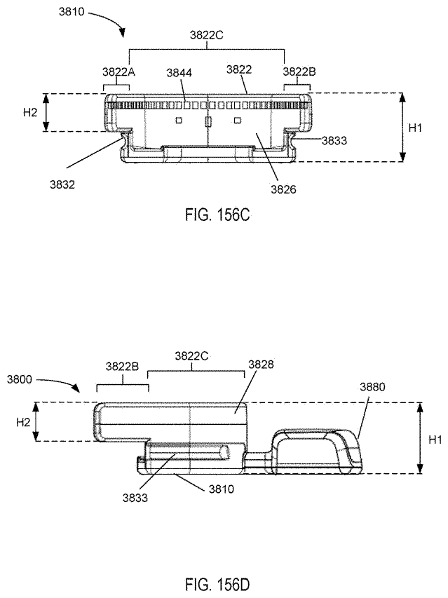

A light therapy apparatus 3800 according to an embodiment is illustrated in FIGS. 156A-156D. The light therapy apparatus 3800 can be included in a light therapy system that is similar in many respects to the light therapy systems described herein, such as with respect to FIGS. 84-113 and 126-141. The light therapy apparatus 3800 is configured to irradiate light in any suitable manner described herein, including, for example, to irradiate the alveolar mucosa and/or tooth root area of the patient, such as via the oral mucosa, for example. Similarly stated, the light therapy apparatus 3800 is configured to administer light therapy to the root of a patient's teeth and/or oral mucosa. More specifically, the light therapy apparatus 3800 is configured to administer light to the patient's teeth and/or oral mucosa sufficient to reduce the overall treatment time for the patient when using one or more appliances that exert a force on one or more of the patient's teeth.

The light therapy apparatus 3800 (and any light therapy apparatus described herein) is useful in combination with traditional orthodontic treatment with an orthodontic appliance, such as brackets and wires, an aligner, or the like. In some embodiments, the light therapy apparatus 3800 is useful prior to treatment with an orthodontic appliance. In some embodiments, the light therapy apparatus 3800 is useful simultaneously with treatment with an orthodontic appliance. For example, the light therapy apparatus 3800 can be disposed within the patient's mouth concurrently with the orthodontic appliance being disposed within the patient's mouth. In some embodiments, the light therapy apparatus 3800 can be disposed within the patient's mouth such that the light therapy apparatus generally is disposed around or over at least a portion of the orthodontic appliance. In some embodiments, the light therapy apparatus 3800 is useful after treatment with an orthodontic appliance. For example, the light therapy apparatus 3800 can be disposed within the patient's mouth for a period of time during which the orthodontic appliance is removed from or otherwise not present within the patient's mouth. In some embodiments, for example, the light therapy apparatus 3800 is useful during a period of time when a patient has removed a removable appliance (such as an aligner) from the patient's mouth. Furthermore, in one or more embodiments, any light therapy apparatus shown and described herein can be useful (e.g., used in combination) with any suitable orthodontic appliance, including, but not limited to, one or more substantially transparent aligners. Such aligners are orthodontic appliances useful for moving a patient's teeth and can adhere, be disposed over, and/or generally conform to one or more of the patient's teeth. In some embodiments, the aligners comprise one or more substantially transparent, removable trays that fit over one or more of the patient's teeth. Each aligner from the set of aligners can be worn by the patient in a predetermined sequence or order, and sometimes for a specified amount or period of time. In particular instances, the aligner (or tray) generally conforms to a patient's teeth but is slightly out of alignment with the starting (e.g., initial) tooth configuration. The aligner can exert a force on one or more teeth (e.g., due to the slight misalignment of the aligner with respect to the one or more teeth).

The orthodontic appliance (e.g., an aligner or brackets and wires) can be configured to exert a force on one or more of the patient's teeth in an amount (or magnitude) effective to move at least one tooth of the patient towards alignment. In some embodiments, the orthodontic appliance is configured to exert a force to move a patient's teeth laterally, e.g., to minimize or close a gap or space between the patient's teeth. In some embodiments, the orthodontic appliance is configured to exert a force to move a patient's teeth rotationally, e.g., to align the patient's teeth. For example, the orthodontic appliance can be configured to exert an orthodontic force, a less-than-orthodontic force, or a heavy force, as described in detail herein, or a combination thereof, on one or more of the patient's teeth in an amount (or magnitude) effective for tooth movement (e.g., towards alignment or to minimize or close a gap between the patient's teeth).

The light therapy apparatus 3800 comprises an intra-oral housing 3810 (also referred to herein as a “mouthpiece”). The intra-oral housing 3810 of the light therapy apparatus 3800 is configured to be disposed in an oral cavity (e.g., in the mouth, not shown in FIGS. 156A-156D) of a patient. The intra-oral housing 3810 can be configured to be electronically and/or physically coupled to an external controller. In one or more embodiments, for example, the intra-oral housing 3810 is configured to be coupled to an extra-oral housing 3880 (also referred to as an “external housing”) that is disposed externally to the patient's mouth when the intra-oral housing (or mouthpiece) 3810 is disposed within the patient's mouth. The extra-oral housing 3880 can be similar in many respects, or identical, to any extra-oral housing described herein (e.g., the extra-oral housing 2560 described herein with respect to FIGS. 84-113), and thus is not described in detail with respect to light therapy apparatus 3800. In other embodiments, the intra-oral housing 3810 is configured to be coupled to the controller in a different manner, such as via a wire, cable connector, or the like (not shown in FIGS. 156A-156D).

The light therapy apparatus 3800 is configured to administer light therapy to the upper jaw and/or the lower jaw of the patient. In other words, the light therapy apparatus 3800 can be configured to administer light therapy with respect to the patient's upper jaw when the apparatus is in a first (e.g., an upright position) in which the light therapy apparatus is disposed within the patient's mouth with respect to the upper jaw (e.g., as shown in FIG. 156C), and can be configured to administer light therapy with to the patient's lower jaw when the apparatus is in a second (e.g., an inverted) position in which the light therapy apparatus is disposed within the patient's mouth with respect to the lower jaw (not shown in FIGS. 156A-156D). As such, the mouthpiece 3810 can be selectively disposed within the patient's oral cavity with respect to each of the upper jaw and the lower jaw of the patient. Similarly stated, the mouthpiece 3810 is configured to be positioned within the mouth such that light therapy can be administered via the mouthpiece 3810 to the upper jaw and the lower jaw, respectively, as described herein, thus eliminating the need for a separate mouthpiece dedicated to the upper jaw or the lower jaw, respectively. It should be noted that although the light therapy apparatus 3800 generally, and the mouthpiece 3810 specifically, can be described as being in the upright position when configured to be oriented with respect to the upper jaw and in the inverted position when configured to be oriented with respect to the lower jaw, in other embodiments, the light therapy apparatus 3800 and the mouthpiece 3810 are in the upright position when configured to be oriented with respect to the lower jaw of the patient, and in the inverted position when configured to be oriented with respect to the upper jaw of the patient.

Generally described with respect to FIGS. 156A-156D, the mouthpiece 3810 comprises a bite tray 3812, a flange 3822, and one or more light emitters 3844. The mouthpiece 3810 can optionally comprise a support plate (not shown in FIGS. 156A-156D) similar or identical to the support plate 2554 of mouthpiece 2510.

The bite tray 3812 is configured to facilitate proper positioning of the mouthpiece 3810 within the patient's mouth. The bite tray 3812 generally defines a lower portion of the mouthpiece 3810. The bite tray 3812 is substantially U-shaped, as seen in FIG. 156B. The bite tray 3812 comprises a bite pad 3814, posterior ends 3812A, 3812B (e.g., the “free” ends of the U shape) and an anterior end 3812C. In some embodiments, the posterior ends 3812A, 3812B each extend from about 1 cm to about 3 cm (including all values and subranges in between) beyond the bite pad 3814, and are useful for latching, hooking, and/or generally securing the tray to the patient's posterior teeth (e.g., each end 3812A, 3812B can hook onto a terminal posterior tooth). The extent/degree of flexibility and/or the nature of flexibility (e.g., inward, towards the patient's mouth) of the posterior ends 3812A, 3812B can be the same as or different than the rest of the bite tray 3812. The bite pad 3814 of the bite tray 3812 is configured to be disposed adjacent to and/or contact an occlusal surface of at least one tooth of the patient when the mouthpiece 3810 is disposed within the patient's mouth. In some embodiments, the bite tray 3812 is configured to receive at least a portion of one or more of the patient's teeth. Stated another way, in some embodiments, the bite tray 3812 defines a recess within which at least a portion of one or more teeth can be disposed or received when the mouthpiece is disposed within the patient's mouth. The posterior ends 3812A, 3812B generally delineate posterior edges of the bite tray 3812 in a posterior portion of the mouthpiece 3810.

The bite tray 3812 can be similar in many respects to the bite tray 2512 described with respect to FIGS. 84-113, and thus is not described in detail with respect to the mouthpiece 3810. For example, the bite pad 3814 of the bite tray 3812 can have any thickness suitable for receiving a bite force thereon, including a constant or spatially varied thickness as described with respect to bite pad 2514. In another example, the bite tray 3812 (and/or bite pad 3814) can be of any suitable dimensions, including those described herein with respect to bite tray 2512 (and/or bite pad 2514, respectively), and can be constructed of any suitable material, including those described herein with respect to bite tray 2512 (and/or bite pad 2514).

As shown in FIGS. 156A-156B, a surface (e.g., an upper) surface of the bite pad 3814 comprises a ridge 3818. The ridge 3818 is disposed along a midline of the mouthpiece 3810 and is elevated with respect to the surface of the bite tray 3812 and/or bite pad 3814. The ridge 3818 can be extended or disposed across a width (or a portion thereof) of the bite pad 3814 (e.g., from an outer perimeter portion of the bite tray 3812 to an inner perimeter portion of the bite tray 3812). The ridge 3818 facilitates positioning of the mouthpiece 3810 within the patient's oral cavity. For example, the mouthpiece 3810 can be configured to be positioned within the patient's oral cavity such that the ridge 3818 is disposed between the patient's front central incisors (on either the upper jaw or the lower jaw). Proprioception of the patient related to the teeth and periodontium can produce sensory feedback to the patient regarding the position of the ridge 3818 of the mouthpiece 3810.

In this manner, the ridge 3818 facilitates centering of the mouthpiece 3810 within the oral cavity, thus promoting symmetry of a light therapy treatment on the alveolar mucosa, or other oral tissue, on both sides of the patient's mouth. In other words, in order to promote the symmetrical administration of light therapy to the root area, the mouthpiece 3810 can be positioned in the patient's mouth with the midline of the mouthpiece 3810 seated along the sagittal plane or within (i.e., plus or minus) 5 degrees of the sagittal plane, and the ridge 3818 can facilitate such positioning in use. The ridge 3818 can have any suitable shape, including, for example, the shape of an inverted V (when seen in the view of FIG. 156B), such that the point or center of the V can be disposed between the patient's front central incisors.

The flange 3822, described in more detail herein, generally defines an upper portion of the mouthpiece 3810. The flange 3822 is coupled to the bite tray 3812. In some embodiments, at least a portion of the flange 3822 is coupled to the outer perimeter portion of the bite pad 3814. The flange 3822 is extended or protruded from the bite tray 3812, as illustrated in FIGS. 156A-156D and described in more detail herein. The upper portion (e.g., the flange 3822) of the mouthpiece 3810 is disposed transversely with respect to the bite pad 3814. As such, when the mouthpiece 3810 is disposed within the patient's mouth, the bite tray 3812 is positioned within the mouth such that the bite pad 3814 of the bite tray 3812 is adjacent the occlusal surface of one or more teeth, and the flange 3822 is disposed between the one or more teeth and buccal tissue. Stated another way, the flange 3822 is configured to be disposed adjacent a portion of a side of the patient's teeth and/or adjacent the alveolar mucosa, when the mouthpiece 3810 is disposed within the patient's mouth, such that the bite tray 3812 is adjacent an occlusal surface of the patient's teeth. For example, the flange 3822 can be disposed adjacent a portion of a buccal side of the patient's teeth and/or adjacent a buccal side of the alveolar mucosa. In this manner, one or more light emitters 3844 enclosed in the flange 3822, as described in more detail herein, are configured to administer light to the patient's teeth and/or alveolar mucosa (e.g., towards the buccal side of the patient's teeth and/or alveolar mucosa). Although the flange 3822 is shown and described herein as a single flange, in other embodiments, the mouthpiece 3810 can comprise two or more flanges.

The flange 3822 is generally U-shaped, e.g., in a manner similar to the bite tray 3812. The flange 3822 comprises two posterior portions 3822A, 3822B and an anterior portion 3822C. The posterior portions 3822A, 3822B of the flange 3822 can be characterized as the portions of the flange 3822 that extend, protrude, project, and/or jut out beyond the posterior ends 3812A, 3812B, respectively, of the bite tray 3812. Generally, the anterior portion 3822C of the flange 3822 can be characterized as the portion of the flange that is coupled to the bite tray 3812. Stated another way, the bite tray 3812 has a U-shaped outer perimeter portion and a U-shaped inner perimeter portion. The outer perimeter portion of the bite tray 3812 is coupled to the flange 3822. The length of the outer perimeter portion of the bite tray 3812 is less than the length of the flange 3822 from each opposing end of the posterior portions 3822A and 3822B of the flange 3822.

During use, in some embodiments, the posterior portions 3822A, 3822B of the flange 3822 can be disposed adjacent one or more posteriorly-located teeth, such as one or more molars and/or one or more bicuspids. In this manner, the posterior portions 3822A, 3822B are useful for treating tissue in the narrower/smaller posterior areas of the oral cavity that cannot otherwise be reached by virtue of the presence of the bite tray 3812, such as in the region of the molars, in the region of the second molar, and/or the like. In some embodiments, each posterior portion 3822A, 3822B of the flange 3822 extends distally beyond the respective posterior end 3812A, 3812B of the bite tray 3812 by any suitable distance such as, but not limited to, a non-zero distance that is less than about 1 cm, about 1 cm, about 2 cm, about 3 cm, about 4 cm, including all subranges and values in between.

In some embodiments, as illustrated in FIGS. 156C-156D, the anterior portion 3822C of the flange 3822 has a height H1, and the posterior portions 3822A, 3822B each have a height H2. In some embodiments, the height H2 of the posterior portions 3822A, 3822B is less than the height H1 of the anterior portion 3822C. In some embodiments, the height H1 of the anterior portion 3822C of the flange 3822 can be about 20 mm, about 25 mm, about 30 mm, about 35 mm, including all subranges and values in between. In some embodiments, the height H2 of each of the posterior portions 3822A, 3822B of the flange 3822 can be about 15 mm, about 12 mm, about 10 mm, about 8 mm, about 5 mm, including all subranges and values in between. While shown in FIG. 156D as a sharp decrease in height from H2 to H1, in other embodiments, any suitable transition in height between the anterior portion and the posterior portions is possible including, but not limited to, a stepwise transition, a gradual (e.g., sloped or curved) transition, and/or the like. In this manner, the narrowing of the flange 3822 in the posterior portions 3822C enhances quality of fit and use of the apparatus 3800.

The flange 3822 is configured to be disposed between the buccal tissue and the alveolar mucosa. Thus, in use, the flange 3822 displaces oral soft tissue to maintain the desired position of the light emitters 3844 relative to the anatomy of the patient. More specifically, the flange 3822 is configured to displace buccal tissue away from the patient's alveolar mucosa. In one or more embodiments, an inner face 3826 of the flange 3822 can be spaced apart from the patient's alveolar tissue when the mouthpiece 3810 is disposed within the patient's mouth and the flange 3822 is displacing the buccal tissue. In one or more embodiments, at least a portion of the inner face 3826 of the flange 3822 can contact the patient's alveolar tissue when the mouthpiece 3810 is disposed within the patient's mouth and the flange 3822 is displacing the buccal tissue.

The flange 3822 of the mouthpiece 3810 is configured to be flexible and/or deformable. Similarly stated, the flange 3822 is constructed from a material and have geometrical dimensions and/or configurations to provide the desired flexibility, as described herein. In this manner, the mouthpiece 3810 can be easily disposed within the oral cavity for a variety of different patients having a variety of different anatomical structures, as described herein.

In some embodiments, the flexibility of the anterior portion 3822C is different than that of the posterior portions 3822A, 3822B. Stated another way, the flange 3822 can have differential flexibility from the anterior portion to the posterior portions, and vice versa. In some embodiments, the posterior portions 3822A, 3822B can exhibit greater flexibility than the anterior portion 3822C, while in other embodiments, the anterior portion 3822C has greater flexibility than the posterior portions 3822A, 3822B. In some embodiments, the change in flexibility between the anterior portion 3822C and the posterior portions 3822A, 3822B can be discrete at the boundary therebetween. For example, the anterior portion 3822C can be composed of a first material having a first measure of flexibility (e.g., of flexural modulus), and the posterior portions 3822A, 3822B can be composted of a second material having a second measure of flexibility different than the first measure of flexibility. As another example, both the anterior portion 3822C and the posterior portions 3822A, 3822B can be made of the same material, but have different densities. In some embodiments, the change in flexibility between the anterior portion 3822C and the posterior portions 3822A, 3822B can be continuous and/or gradual, i.e., the rate of change of flexibility from one portion to another can be below a predetermined threshold. For example, both the anterior portion 3822C and the posterior portions 3822A, 3822B can be made of the same materials, and a density gradient exists that results in a gradient in flexibility from one portion to another.

In some embodiments, the flexibility of the posterior portions 3822A, 3822B can be greater than that of the anterior portion 3822C. In some embodiments, the flexibility of the posterior portions 3822A, 3822B permits deflection of the posterior portions 3822A, 3822B in multiple ways, including: a) in a manner similar to that of the anterior portion 3822C deflecting towards and away from the bite tray 3812 (movement denoted by arrow Fa in FIG. 156A); b) in a torsional/twistable manner (e.g., up to a torsion angle of about 30 degrees), about the point of attachment/coupling/formation with the anterior portion 3822C (movement denoted by arrow Fₜ in FIG. 156A); and/or c) in a “flapping”/flappable manner (e.g. up to an angle of about 30 degrees), towards or away from a midline of the oral cavity (or away from the tongue towards buccal tissue) of the patient during use (movement denoted by arrow Fr in FIG. 156A). In this manner, the posterior portions 3822A, 3822B provide enhanced flexibility for conforming to the patient's mouth in the relatively narrow reaches of the posterior portions of the patient's mouth, near and/or posterior to the molars.

The mouthpiece 3810 defines at least one groove 3832, 3833 defined by a lower outer (or front) surface of the flange 3822. For example, the mouthpiece 3810 comprises the first groove 3832 and the second groove 3833, each defined by the outer, anterior, or front surface 3828 of the mouthpiece 3810. The grooves 3832, 3833 can each be similar in many respects, or identical, to grooves 2532, 2533 described with respect to mouthpiece 2510 and FIGS. 84-113. In some embodiments, the grooves 3832, 3833 are each disposed at a height between the bite pad 3814 and a lower edge of a flexible circuit board disposed within the flange 3822, described in more detail below (not shown in FIGS. 156A-156D). Stated another way, the grooves 3832, 3833 can be defined by a base portion the flanges 3822. The grooves 3832, 3833 each extend about the outer surface 3828 of the mouthpiece 3810 between the posterior end portion of the mouthpiece 3810 and an anterior portion of the mouthpiece 3810. As illustrated, in some embodiments, the grooves 3832, 3833 are formed on the anterior portion 3822C of the flange 3822A, 3822B and are absent from the posterior portions 3822A, 3822B. In other embodiments, however, the grooves 3832, 3833 can be formed in a manner where they are defined by or extend at least partially along their respective posterior portions 3822A. 3822B. In some embodiments, a portion of the grooves 3832, 3833 formed in the posterior portions 3822A, 3822B can be relatively narrower and/or have a different cross-sectional profile than a portion of the grooves 3832, 3833 formed in the anterior portion 3822C.

As best seen in FIG. 156D, the grooves 3832, 3833 can be spaced apart at the anterior end portion of the mouthpiece 3810. In other words, the grooves 3832, 3833 do not necessarily meet at the anterior end of the mouthpiece 3810. For example, a first portion of the anterior portion 3822C of the flange 3822 can exclude the grooves 3832, 3833 and a second portion of the anterior portion 3822C of the flange 3822 can comprise the grooves 3832, 3833. The grooves 3832, 3833 can be noncontiguous and/or do not share a common boundary. The grooves 3832, 3833 can have any suitable shape, including, for example, that of a semi-circle or U-shape. The grooves 3832, 3833 produce a hinge-like structure (i.e., a “living hinge”) about which the flange 3822 can rotate, bend and/or deflect. In this manner, the grooves 3832 permits the flanges 3122, 3124 to deflect inwardly (e.g., generally towards a midline of the apparatus 3800, or more specifically towards a midline of the bite tray 3812), for example, in response to pressure from the patient's lip or inner cheek.

As such, the groove 3832 facilitates the transition of the mouthpiece 3810 between a first configuration and a second configuration. When the mouthpiece 3810 is in the first configuration, the angle formed between the anterior portion 3822A. 3822B of the flange 3822 and the bite pad 3814 (the “outer flange angle”) has a first value. When the mouthpiece 3810 is in the second configuration, the outer flange angle has a second value that is different from the first value. In particular, the mouthpiece 3810 can be moved to the second configuration when disposed within the patient's mouth. In one or more embodiments, the second value is less than the first value (i.e., the anterior portion 3822C of the flange 3822 “tips” inwardly toward the bite plate 3812 when the mouthpiece 3810 is inserted into the mouth). In one or more embodiments, the outer flange angle is approximately 90 degrees when the mouthpiece is in the first configuration and is acute when the mouthpiece is in the second configuration. In one or more embodiments, the outer flange angle is about 80 degrees (e.g., the flange 3822 tips inward toward the bite plate 3812 by about 10 degrees) when the mouthpiece is in the second configuration. In other embodiments, the outer flange angle is between about 75 degrees and about 80 degrees (e.g., the flange 3822 tips inward toward the bite plate 3812 by between about 10 degrees and 15 degrees). In yet other embodiments, the outer flange angle is about 85 degrees, about 75 degrees, about 70 degrees, or about 65 degrees (e.g., the flange 3822 tips inward toward the bite plate by about 5 degrees, about 15 degrees, about 20 degrees and about 25 degrees, respectively) when the mouthpiece is in the second configuration.

The flexibility of the mouthpiece 3810, and the differential flexibility of the various portions of the flange 3822 in particular, provides significant advantages. For example, in contrast to mouthpieces constructed of a hard plastic and/or with a permanent set (or shape), the current arrangement allows for easier insertion and better conformance to the oral tissue of the patient. The flexibility of the mouthpiece 3810 also accommodates variation in patient anatomy (whether between two different patients or for the same patient as that patient's anatomy changes over time). For example, some patients have a pronounced overbite and might need more or less than a 10 degree inward deflection (or “tip-in”). In such instances, the mouthpiece 3810 can conform to the internal structure and/or anatomy within the patient's mouth. As another example, over the course of orthodontic treatment, the patient's dental anatomy is expected to change. Accordingly, the mouthpiece 3810 can conform to the internal structure and/or anatomy within the patient's mouth to accommodate such change without requiring new mouthpiece moldings or the like. Further, the flexible design of the mouthpiece 3810 provides greater comfort for the patient than would be provided by mouthpieces constructed of a hard plastic.

Additionally, the flexible nature of the mouthpiece 3810 and/or the flange 3822 provides manufacturing benefits. In particular, fabrication and/or molding of a mouthpiece having an acute angle between the bite surface and the side surface of the flange (i.e., the internal angle of the flange or the “flange angle”) can be difficult. The design of the mouthpiece 3810, however, allows for the molding and/or fabrication to be performed with a flange angle of approximately ninety degrees (or greater), while allowing for an in-use inner flange angle that is acute (e.g., when the mouthpiece 3810 is in the second configuration, as described herein).

The mouthpiece 3810 of the light therapy apparatus 3800 can comprise an electronic assembly. The electronics assembly is disposed primarily in the flange 3822, and comprises a flexible circuit board (not shown) and comprises or is otherwise coupled to the one or more light emitters 3844. The one or more light emitters 3844 are coupled to the flange 3844 of the mouthpiece 3810. In some embodiments, one or more light emitters 3844 are disposed on, disposed in, or embedded in the flange 3822, as described in more detail herein. In some embodiments, light emitter(s) 3844 protrude through and/or are generally visible on a posterior (or lingual-facing) surface of the flange 3822.

In some embodiments, and as illustrated in FIGS. 156A-156D, the one or more light emitters 3844 can comprise multiple light emitters disposed on or at least partially in each of the posterior portions 3822A, 3822B and the anterior portion 3822C of the flange. In other embodiments, the light emitters 3844 can be disposed on the anterior portion 3822C, and be absent from the posterior portions 3822A, 3822B. In some embodiments, at least one light emitter 3844 is disposed on one or more of the posterior portions 3822A, 3822B. In some embodiments, the light emitter(s) disposed on the posterior portions 3822A, 3822B are not co-linear with the light emitter(s) disposed on the anterior portion 3822C (as illustrated), but are offset in a direction towards or away from the bite tray 3812. For example, the light emitter(s) coupled to the posterior portions 3822A, 3822B of the flange 3822 can be arranged in one or more rows that are parallel to, but not co-linear to, the one or more rows of light emitter(s) 3844 coupled to the anterior portion 3822C of the flange 3822.

The light emitter(s) can be any suitable light emitter described herein, such as a plurality of LEDs. The light emitters 3844 are electrically and/or physically coupled to the flexible circuit board, which in turn electrically couples the light emitters 3844 to electronic circuitry outside of the mouthpiece 3810 (e.g., in the extra-oral housing 3880 or via electrical connectors to an external controller, not shown). In this manner, the light emitters 3844 can receive power and/or a signal to produce the desired light, as described herein.

The light emitters can be disposed on the flexible circuit board in a manner as already described for FIG. 131, and will not be further detailed herein. In this manner, the light emitters 3844 are configured to emit light toward a patient's teeth and/or adjacent oral tissue when the mouthpiece 3810 is disposed within the patient's mouth. Stated another way, the light emitters 3844 are configured to emit light towards the anterior root area of an upper and/or lower jaw and/or the buccal alveolar soft tissue.

The light emitters 3844 can be configured to emit light at any suitable intensity, wavelength and/or frequency described herein. For example, in one or more embodiments, the light emitters 3844 can be configured to emit light in the red, the infrared, or near infrared wavelength range. For example, in one or more embodiments, the light emitters 3844 are configured to emit light at a wavelength of about 850 nm. In one or more embodiments, the light emitters 3844 are configured to emit light at a wavelength of 850 nm±5 nm. In some embodiments, one or more of the light emitters 3844 are configured to emit light at a first wavelength, and the remainder of the light emitters 3844 are configured to emit light at a second wavelength. In some embodiments, the first wavelength is about 850 nm, and the second wavelength is about 625 nm.

In some embodiments, at least about 10%, at least about 20%, at least about 30%, at least about 40%, at least about 60%, at least about 80%, at least about 90% of the light emitters 3844 emit light at the first wavelength (including all values and sub-ranges in between), and the remainder emit light at the second wavelength. In some embodiments, about two-thirds of the light emitters 3844 can emit light at the first wavelength, and the remaining one-third of the light emitters 3844 can emit light at the second wavelength. It is understood that while explained herein with reference to the light emitters emitting two different wavelengths, any suitable number of wavelengths can be employed in this manner.

The light emitters 3844 can be configured to emit light sufficient deliver light energy to the patient's bone to facilitate and/or perform any of the methods described herein. The light emitters 3844 can be configured to emit light at less than 150 mW/cm².

In some embodiments, the light emitters 3844 are coupled to the electronics assembly such that zones of the light emitters are individually addressable or controllable. One or more of the light emitters 3844 (e.g., one or more LEDs) can constitute a zone of light emitters. In one or more embodiments, from about 1 to about 20, from about 1 to about 15, from about 1 to about 10, or from about 1 to about 9, or from about 1 to about 8, or from about 1 to about 7, or from about 1 to about 6, or from about 1 to about 5, or from about 1 to about 4 light emitters are included in a zone of light emitters. It will be understood by one of skill in the art that the number of light emitters that actually irradiate light during light therapy will vary depending on patient presentation and ultimate treatment protocol. In some embodiments, light emitters disposed on each posterior portion 8422A can constitute one or more zones of light emitters, and light emitters disposed on the anterior portion 8422B can constitute a separate zone or zones of light emitters. In some embodiments, light emitters disposed on the anterior portion 8422B can constitute two or more zones of light emitters. In some embodiments, each zone is independently and selectively operable, such that any of the following example scenarios is possible: 1) a first zone of light emitters can emit light at a first time, and a second zone of light emitters can emit light at a second time that does or does not overlap with the first time; and 2) multiples zones of light emitters can be operated to emit light in a predetermined sequence and/or pattern. In some embodiments, the light emitters within a zone can emit light at a wavelength (e.g., at about 850 nm) that is different than that emitted by light emitters in a different zone (e.g., at about 625 nm). In other embodiments, the light emitters within a single zone can emit light at two or more different wavelengths.

The light emitters 3844 can provide any suitable power density effective for treatment. In some embodiments, the power density is from about 30 mW/cm² to about 150 mW/cm², including all values and sub-ranges in between. In some embodiments, the light emitters 3844 can provide a power density of about 60 mW/cm². In some embodiments, the light emitters 3844 can provide a power density of about 120 mW/cm².

The light emitters 3844 can be disposed on the mouthpiece 3810 in any suitable configuration, including any configuration described herein. For example, in one or more embodiments, the light emitters 3844 comprise LEDs disposed in one or more parallel rows and/or columns. In one or more embodiments, at least some of the light emitters 3844 are disposed on the mouthpiece 3810 in a single row, as illustrated in FIGS. 156A-156C. In some embodiments, the mouthpiece 3810 comprises additional light emitters e.g., structurally and/or functionally similar to the light emitters 3050, 3052.

Although the light emitters 3844 are shown as being evenly spaced on the flange 3822, in other embodiments, the light emitters can be unevenly spaced. For example, in one or more embodiments, a mouthpiece can comprise a series of light emitters that are spaced apart by a first distance in or near the anterior portion of the mouthpiece and spaced apart by a second, different distance in or near the posterior portion of the mouthpiece.

The mouthpiece 3810 can be constructed of any suitable material, including, for example, any material described herein with respect to mouthpiece 2510, and thus such material is not described in detail with respect to mouthpiece 3810. For example, the mouthpiece 3110 can be constructed of an elastomeric material (e.g., a soft silicone). In another example, the mouthpiece 3810 can be fabricated from medical-grade injection-molded, highly flexible and very low durometer silicone. In another example, the silicone and/or portions of the mouthpiece 3810 are substantially transparent, such that one or more components embedded within the silicone are visible through the silicone. Moreover, in this manner, the mouthpiece 3810 can provide suitable optical properties for allowing the light produced and/or conveyed by the light emitters 3844 to pass through the mouthpiece 3810 to the desired target tissue. In one or more embodiments, the mouthpiece 3810 and/or the flange 3822 can comprise one or more components configured to filter, focus and/or otherwise act upon the light produced by the light emitters 3844. In other embodiments, the mouthpiece 3810 can comprise air gaps between the light emitters 3844 and the surface of the flange 3822 to facilitate focusing of the light. The mouthpiece 3810 can constructed such that the light emitters 3844 are fully encapsulated or embedded within the molded silicone such that no space or air gap exists between the silicone material and the emitters 844. Similarly stated, the mouthpiece 3810 can exclude an air gap between the light emitters 3844 and the material of the mouthpiece 3810, thus no air gap lensing is needed to produce the desired optical properties of the light produced by the light emitters 3844.

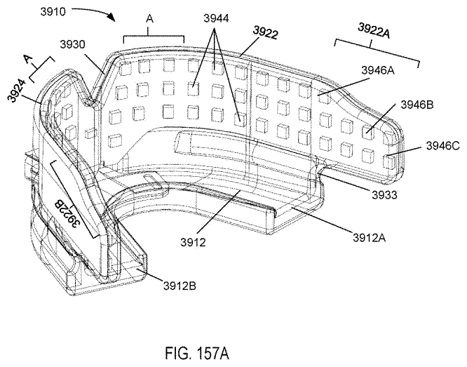

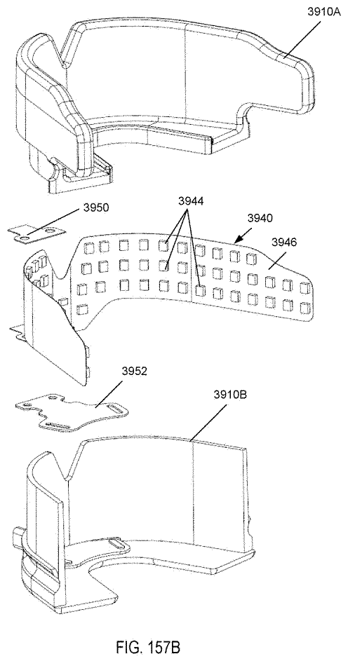

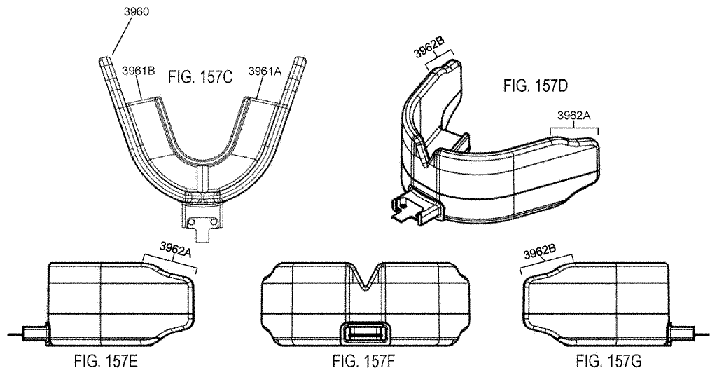

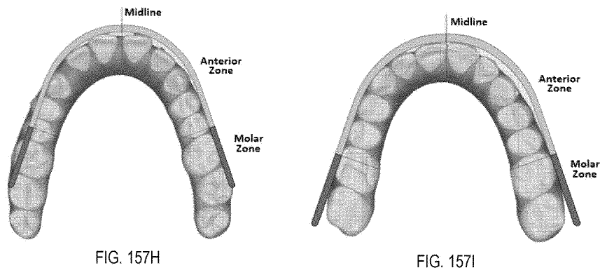

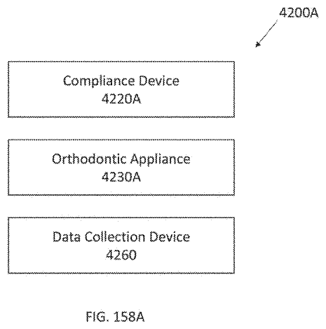

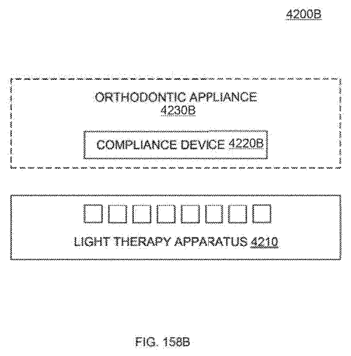

An intra-oral housing 3910 (also referred to herein as a “mouthpiece”) according to an embodiment, being sized and/or shaped to be fully or substantially fully disposed within a patient's mouth, is illustrated in FIGS. 157A-157B. Unless explicitly noted otherwise, components of the mouthpiece 3910 can be structurally and/or functionally similar or identical to similarly named and numbered components of the mouthpiece 3810, or any intra-oral housing or mouthpiece described herein. For example, the intra-oral housing 3910 can comprise light emitters 3944 that can be similar to the light emitters 3844, the intra-oral housing 3910 can comprise a posterior end 3912A of a bite tray 3912 that can be similar to the posterior end 3812A of the bite tray 3812, and so on. As shown in FIG. 157A, the bite tray 3912 comprises two posterior ends: 3912A and 3912B.

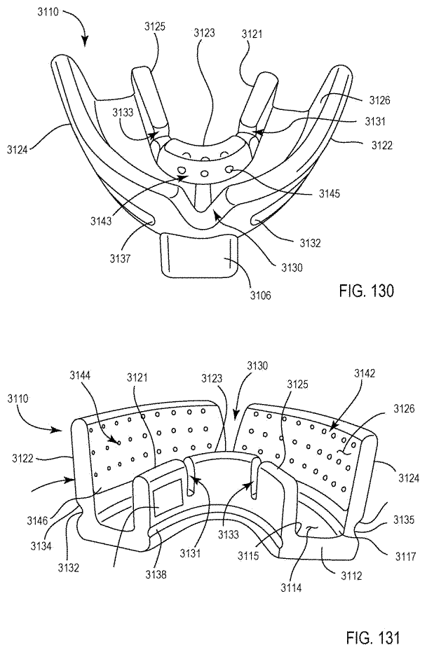

As illustrated in FIG. 157A, the mouthpiece 3910 can comprise a notch 3930, which can be similar to the notch 2530, described herein with respect to FIG. 90, or to notch 3130, described herein with respect to FIG. 130. The notch 3930 can be V-shaped, generally dividing the anterior portion of the mouthpiece into two flanges 3922, 3924, compared to the flange 3822, which has a continuous top edge with no notch formed therein. In this manner, the deflectability (e.g., with respect to the bite tray) of the flanges 3922, 3924 can be enhanced (e.g., as described herein with respect to FIG. 90). In some embodiments, the deflectability of the flange 3922 can be different than the deflectability of the flange 3924.

The light emitters 3944 are disposed along multiple rows and columns, in contrast to the light emitters 3844, which are illustrated as being disposed in a single row. In some embodiments, and as illustrated in FIG. 157A, one or more rows of light emitters 3944 are noncontiguous in the vicinity of the notch 3930, either by virtue of the presence of the notch, or by design. In some embodiments, one or more rows of light emitters 3944 can extend to different extents to the posterior portions 3922A, depending on the design of the posterior portion. For example, FIG. 157A illustrates a tapered design for the posterior portion 3922A, such that a row 3946A of light emitters extends into the posterior portion 3922A to a lesser degree than the rows 3946B, 3946C of light emitters. Stated another way, the number of light emitters in the row 3946A that are disposed in the posterior portion 3922A is fewer than the number of light emitters in the row 3946B of light emitters or in the row 3946C of light emitters that are disposed in the posterior portion 3922A.

As shown in FIG. 157A, posterior portions of the flange (3922A and 3922B) extend (e.g., by a distance of from about 1 mm to about 24 mm, or about 10 mm to about 24 mm, or about 1 mm to about 12 mm, or about 12 mm to about 24 mm, or about 5 mm to about 15 mm, or about 5 mm to about 20 mm, or about 2 mm to about 18 mm, or about 5 mm to about 18 mm, or about 1 mm to about 5 mm, or about 5 mm to about 10 mm) beyond the posterior ends 3912A and 3912B (respectively) of the bite tray. One or both posterior portions of the flange (3922A and 3922B) can have a height (i.e., a measurement from a bottom edge thereof to a top edge thereof, as oriented in FIG. 157A and in a non-deflected state) that is less than a maximum height (i.e., a measurement from a bottom edge thereof to a top edge thereof, taken in a region other than the notched region) of an anterior portion (“A”) of the flange. In some embodiments, a first posterior portion of the flange (3922A) is substantially perpendicular to a first posterior end of the bite tray (3912A) and/or a second posterior portion of the flange (3922B) is substantially perpendicular to a second posterior end of the bite tray (3912B).

In some embodiments, one or both of the posterior portions of the flange (3922A and 3922B) are twistable with respect to an anterior portion (“A”) of the flange. Alternatively or in addition, one or both of the posterior portions of the flange (3922A and 3922B) are flappable with respect to an anterior portion (“A”) of the flange. Although the posterior portions 3922A, 3922B shown and described with reference to FIG. 157A as being tapered or “sloped” (i.e., having a height that gradually reduces along a direction extending from an anterior portion “A” of the flange towards a posterior portion (3922A, 3922B) of the flange), other height variations are also within the scope of the invention. For example, the posterior portions 3922A, 3922B can each have a height that changes in a stepwise manner along a direction extending from an anterior portion of the flange towards the posterior portion of the flange. The height variation can occur along an entire flange (i.e., spanning the distance from a front/anterior of the overall mouthpiece to a rear/posterior of the overall mouthpiece), or can be confined to the posterior portions (3922A, 3922B) of the flange.

The light emitters 3944 can provide any suitable power density effective for treatment. In some embodiments, the power density is from about 30 mW/cm² to about 150 mW/cm², including all values and sub-ranges in between. In some embodiments, the light emitters 3844 can provide a power density of about 60 mW/cm². In some embodiments, the light emitters 3844 can provide a power density of about 120 mW/cm².

FIG. 157B is an exploded view showing components of the mouthpiece 3910. The mouthpiece 3910 comprises an inner layer 3910A, an electronic assembly 3940, and an outer layer 3910B. The mouthpiece also comprises connectors 3950, 3952 with slots formed therein for coupling the layers 3910A, 3910B, and the electronic assembly 3940, as well as for electrical/electronic coupling of the mouthpiece 3910 to an external housing (not shown).

The electronics assembly 3940 comprises a flexible circuit board 3946, and the light emitters 3944. The flexible circuit board 3946 can be formed of any suitable material as any flexible circuit board described herein, such as flexible circuit boards 2546, 3046, 3146, and 3646. The light emitters 3944 can be any suitable light source described herein, including, for example, LEDs.

The inner layer 3910A of the mouthpiece 3910 and the outer layer 3910B of the mouthpiece 3910, in some embodiments, can be formed of substantially the same material such as, for example, any material described herein (e.g., with respect to mouthpiece 2510), and thus such material is not described in detail with respect to mouthpiece 3810. For example, the layers 3910A, 3910B can be constructed of an elastomeric material (e.g., a soft silicone, a silicone rubber such as the Silbione® liquid silicone rubber, and/or the like). In another example, the layers 3910A, 3910B can be fabricated from medical-grade injection-molded, highly flexible and very low durometer silicone. In some embodiments, the layers 3910A, 3910B can be made of the same material but have differing flexibility/rigidity. In some embodiments, the layers 3910A, 3910B can be made of different materials but have substantially the same flexibility/rigidity. In some embodiments, the layers 3910A, 3910B can be made of different materials having differing flexibility/rigidity.

As a non-limiting example of manufacture and assembly of the mouthpiece 3910, in some embodiments, the inner layer 3910 can be formed via injection molding of an elastomeric material. The flexible circuit board 3946 can then be placed inside a volume formed by the inner layer 3910 via an opening in the inner layer 3910. Thereafter, additional elastomeric material can be poured into any residual volume in the inner layer 3910, and can also be used to create the bite tray 3912. The outer layer 3910B can then be coupled to the inner layer 3910A via the connectors 3950, 3952 as described herein.

FIG. 157C is a top view of a light therapy apparatus according to an embodiment. FIGS. 157D-G are perspective, first side, rear, and second side views, respectively, of the light therapy apparatus of FIG. 157C. As shown in FIG. 157C, the light therapy apparatus 3960 comprises a bite tray 3961 having a first posterior end 3961A and a second posterior end 3961B. Visible in each of FIGS. 157D-157G are first and second posterior flange portions 3962A and 3962B, which are similar to posterior portions 3922A, 3922B of FIG. 157A in that they protrude beyond the posterior ends of the bite tray (3961A and 3961B, respectively, but differ in that they have a tapered or “sloped” bottom portion as well as top portion. In some embodiments, a first (upper/top) edge of each of the posterior portions has a taper, shape or contour that mirrors (i.e., has the same shape as, but is inverted with respect to) a taper, shape or contour of a corresponding second (lower/bottom) edge of each of the posterior portions. In alternative embodiments, a first (upper/top) edge of each of the posterior portions has a taper, shape or contour that is different from a taper, shape or contour of a corresponding second (lower/bottom) edge of each of the posterior portions. Although the posterior portions 3962A, 3962B shown and described with reference to FIGS. 157D-G as being tapered or “sloped” (i.e., having a height that gradually reduces along a direction extending from an anterior end of the flange towards a posterior end of the flange), other height variations are also within the scope of the invention. For example, each of the posterior portions 3962A, 3962B can each have a first (upper/top) edge that reduces in height (with respect to a midline of the light therapy apparatus when viewed as shown in FIG. 157D) in a stepwise manner along a direction extending from an anterior end of the flange towards the posterior end of the flange, in which case the corresponding second (lower/bottom) edge of each of the posterior portions can have a shape that either mirrors that of the first edge, or that differs from that of the first edge. For example, the first edge can taper (e.g., downward) in a stepwise manner, while the second edge tapers (e.g., upward) in a sloped manner.

In some embodiments, a method for regulating tooth movement comprises administering light (e.g., using a light therapy apparatus similar to that of shown in any of FIGS. 157A-157G) to a region of alveolar mucosa overlying a root of a tooth (e.g., a molar) of a patient such that a rate of movement of the tooth is increased (in the presence of the light), relative to a rate of movement of the tooth in the absence of the light. The region of alveolar mucosa can include alveolar mucosa overlying a tooth root of a tooth such as a molar. In some such embodiments, the tooth is a first tooth, and the rate of movement is associated with a movement of the first tooth relative to a second tooth of the patient. The method can also comprise removably coupling an orthodontic appliance to the tooth.

FIGS. 157H-I show example views of a patient's mouth (upper dental arch and lower arch), with an overlaid outline of a light therapy apparatus disposed therein, and illustrating the positioning of a midline, an anterior zone, and a molar zone of the light therapy apparatus. The light therapy apparatus of FIGS. 157H and 157I can be any intra-oral light therapy apparatus described herein. A “molar zone” can comprise one or more light emitters, and in some embodiments, one or both of the molar zone regions of the light therapy apparatus can be controlled separately from other light-emitting portions of the light therapy apparatus. The precise positioning of the light therapy apparatus within the patient's mouth can vary, depending, for example, of the patient's anatomy (e.g., tooth size, age, etc.), however, as shown in FIGS. 157H and 157I, the molar zone of the light therapy apparatus can span at least the entirety of the length of a first molar and a portion (e.g., at least 50% coverage) of the second molar, such that, during use, light from the light therapy apparatus is directed to alveolar mucosa overlying tooth roots of the associated molars. Such embodiments can enhance clinical efficacy in terms of inducing accelerated movement of the teeth orthodontically, as compared with treatments that do not involve such illumination. In some embodiments, light emitted from the molar zone to alveolar mucosa overlying tooth roots of the associated molars can spread in the jaw bone.

In some embodiments, a light therapy apparatus (or a flange thereof) comprises two controllable light-emitting zones: a molar zone (e.g., as described herein with reference to FIGS. 157H-I), and an anterior zone that excludes the molars (or associated alveolar mucosa). The molar zone can comprise one or more light emitters (e.g., LEDs) configured to emit light to a patient's molars or anatomy associated with the molars (e.g., to alveolar mucosa overlying tooth root associated with molars), for example, for orthodontic treatment, e.g., using one or more aligners, and/or to distalize molars (i.e., sequentially move molars posteriorly) for the treatment of malocclusion. The controllability of the light-emitting zones can be accomplishes, for example, via a Bluetooth connection and/or one of a mobile software application (“app”) or web-based app. In some embodiments, the light therapy is controllable remotely (e.g., via an application programing interface, “API”), for example via the mobile or web-based app, such that the molar zone and anterior zone can be programmed to emit light according to a predetermined schedule, such as a tooth movement plan.

In some embodiments, each molar zone resides solely within a posterior portion, such as posterior portions 3922A, 3922B of FIG. 157A. In other embodiments, each molar zone resides partially within a posterior portion of an associated flange of the light therapy apparatus, such as posterior portions 3922A, 3922B of FIG. 157A, and partially within another portion of the associated flange. In other words, the molar zones can each “straddle” protruding and non-protruding portion of the flange. The molar zones can be configured to emit light to molar ara (i.e., portions of the patient's mouth associated with the molars, such as alveolar mucosa overlying a tooth root of a molar, and/or the molars themselves), for example to accelerate distalization or any other significant molar movement that is planned as part of an orthodontic treatment plan. In some embodiments, the posterior portions 3922A, 3922B of the flange can one or more of: improve the positioning of the light therapy apparatus within a patient's mouth during use, reduce the need for a patient to bite down on the bite tray for positioning purposes, or increase a surface area of regions within the patient's mouth that are irradiated during treatment with the light therapy apparatus.

Although apparatuses or portions thereof (e.g., apparatus 2100, 2500, 3000, 3100, 3300, 3600, 3700, 3800, mouthpiece 3910) are each separately described herein via different figures, aspects of any of the apparatuses, or portions thereof, (e.g., apparatus 2100, 2500, 3000, 3100, 3300, 3600, 3700, 3800, and mouthpiece 3910) can be combined with one or more aspects of any one or more of another of apparatus (e.g., apparatus 2100, 2500, 3000, 3100, 3300, 3600, 3700, 3800, and mouthpiece 3910). For example, the signal processing described for apparatus 2500 can be included or used in apparatus 3100 and/or 3600 as well. In another example, one or more sensors (e.g., the light emitters 3050, 3052) described with respect to apparatus 3000 can be included in apparatus 3800 or mouthpiece 3910.

In some embodiments, a method for regulating tooth movement can comprise intra-orally administering, to one or more teeth of a patient in need thereof, an effective amount of light from one or more light emitters of a light therapy apparatus (e.g., apparatus 2100, 2500, 3000, 3100, 3300, 3600, 3700, 3800, mouthpiece 3910). In some embodiments, the one or more light emitters can be associated with the one or more teeth during use of the light therapy apparatus. For example, a user or practitioner can specify that the one or more light emitters can substantially overlay the one or more teeth of the patient. The one or more light emitters can be selectively employed to deliver the light therapy to the targeted teeth. As another example, the light therapy apparatus can be pre-programmed to associate certain light emitters with certain teeth. In some embodiments, the one or more teeth include one or more molars of the patient. In this manner, aspects of the method are useful for selectively increasing the rate of tooth movement in the one or more teeth (e.g., one or more molars) relative to other teeth of the patient (e.g., relative to one or more incisors and/or canines). The rate of tooth movement for the patient's teeth that are not administered light can be substantially unchanged.

The light emitters can provide any suitable power density effective for treatment, as described herein. In some embodiments, the power density is from about 30 mW/cm² to about 150 mW/cm², including all values and sub-ranges in between. In some embodiments, the light emitters can provide a power density of about 60 mW/cm². In some embodiments, the light emitters can provide a power density of about 120 mW/cm².

In some embodiments, the one or more light emitters can constitute a first zone of illumination of multiple zones of illumination (e.g., two, three, four or more zones of illumination) of the light therapy apparatus (as generally described herein with respect to the mouthpiece 3810). In some embodiments, the one or more light emitters in the first zone of illumination can be at least partly or wholly disposed on a posterior portion of a flange of a mouthpiece of the apparatus that posteriorly extends from or beyond a posterior end of a bite tray of the mouthpiece. For example, as described herein with reference to the apparatus 3800, the light emitters 3844 can comprise multiple light emitters disposed on the posterior portion 3822A that extends beyond the posterior end of the bite tray 3812. In this manner, the light emitters in the first zone of illumination are configured to overlay and emit light to one or more of: a targeted region of alveolar mucosa, or a targeted region of a tooth or teeth, such as one or more molars. Each zone of illumination can be independently controlled by a controller of an electronics assembly of the light therapy apparatus. In some embodiments, the electronics assembly can be configured to receive and/or store (e.g., in memory) an indication (e.g., identifiers, a mapping, etc.) of a plurality of zones of illumination associated with the light therapy apparatus, each zone of illumination comprising a subset of light emitters from a plurality of light emitters of the overall light therapy apparatus. The electronics assembly can be further configured to independently control an operational state (e.g., on, off, standby, sleep mode) of all light emitters (collectively) within a given zone of illumination from the plurality of zones of illumination. As used herein, a “zone of illumination” can refer to a region or portion of the light therapy apparatus that is configured to emit light to a region within a mouth of patient (e.g., to alveolar mucosa above the patient's gums).

It is understood that while described herein with respect to light emitters in a first zone of illumination affecting rate of tooth movement in some teeth, the approach is extendible to treating any desirable tooth or teeth, using one or more zones of illumination.

In some embodiments, methods according to embodiments comprise administering the effective amount of light via one or more light emitters in the first zone of illumination in conjunction with the use of one or more orthodontic appliances, such as transparent aligner(s). In some embodiments, the methods comprise administering light using the light therapy apparatus for a predetermined time period. More specifically, during a particular session of a treatment program, the patient (or other administrator or healthcare professional) can use the light therapy apparatus to administer light therapy via one or more light emitters (e.g., in the first or a different zone of illumination) for a predetermined duration of time, i.e., the predetermined time period. In other words, the predetermined time period can be associated with an individual treatment session. In some embodiments, the predetermined time period is less than 1 minute, is about 1 minute, is about 2 minutes, is about 4 minutes, is about 6 minutes, is about 8 minutes, is about 10 minutes, including all values and sub-ranges in between. In some embodiments, the predetermined time period is about 5 minutes. In some embodiments, the predetermined time period is about 2.5 minutes. In some embodiments, the methods comprise administering light via a light therapy apparatus during a first session having a first predetermined time period and a during a second session having a second predetermined time period, where the second session is non-consecutive with the first session, and the duration of the first predetermined time period is independent of the duration of the second predetermined time period (note that the first predetermined time period can have a duration the same as or different from a duration of the second predetermined time period).

In some embodiments, the power density of the LED emitters, for example, during a particular treatment session or portion thereof, is about 60 mW/cm², and the predetermined time period is about 5 minutes. In some embodiments, the power density of the LED emitters is about 120 mW/cm², and the predetermined time period is about 5 minutes. In some embodiments, the power density of the LED emitters is about 60 mW/cm², and the predetermined time period is about 2.5 minutes. In some embodiments, the power density of the LED emitters is about 120 mW/cm², and the predetermined time period is about 2.5 minutes.

In some embodiments, the methods comprise removably coupling an orthodontic appliance to the one or more teeth of the patient prior to use of the light therapy apparatus as described herein, i.e., prior to the predetermined time period. The orthodontic appliance can include, for example, a removable aligner. In some embodiments, the methods comprise removing the orthodontic appliance prior to the predetermined time period. In some embodiments, the orthodontic appliance is or remains removably coupled to the teeth of the patient during the predetermined time period, and can be removed after expiration of the predetermined time period. In some embodiments, the methods comprise applying and subsequently removing the orthodontic appliance after the predetermined time period.

In some embodiments, the orthodontic appliance is one of a set of orthodontic appliances (e.g., is a first orthodontic appliance) that the patient has been prescribed to use. For example, the set of orthodontic appliances can include a set of removable and/or transparent aligners useful for moving one or more teeth of the patient towards alignment upon successive and compliant use of the set of aligners by the patient. In some embodiments, the method can comprise, subsequent to removably coupling the first orthodontic appliance to the teeth of the patient, removing the first orthodontic appliance, and removably coupling a second orthodontic appliance of the set of orthodontic appliances of the patient. In some embodiments, for example, light therapy can be administered using the light therapy apparatus when the first orthodontic appliance is coupled to the teeth (or otherwise disposed within the mouth of) the patient, and light therapy subsequently can be administered using the light therapy apparatus when the second orthodontic appliance is coupled to the teeth (or otherwise disposed within the mouth of) the patient. The timing for the coupling and/or subsequent removal of the second orthodontic appliance can be before, during, or after the predetermined time period in a manner consistent with the prior use of the first orthodontic appliance. Stated another way, in some embodiments, the second orthodontic appliance can be used in a manner similar to the first orthodontic appliance

Methods for Regulating Tooth Movement

Methods for regulating tooth movement are provided herein. Such methods comprise administering an effective amount of light to a patient, wherein the effective amount of light is irradiated from the emitter of an apparatus of the invention. In one or more embodiments, at least a portion of the apparatus contacts the alveolar soft tissue (e.g., the alveolar mucosa) when the light is administered. In one or more embodiments, at least a portion of the apparatus does not contact, but is at a particular distance (e.g., from 0.1 cm to 3 cm), from the alveolar soft tissue when the light is administered. As is described in more detail herein, the light is administered using any one of the intra-oral apparatuses of the invention. In one or more embodiments, the methods also comprise allowing a force, in one or more embodiments, a heavy force, to be exerted on one or more teeth of the patient in need thereof, wherein the light is administered before, during or after the force is exerted.

Other embodiments of the invention provide methods for reducing, minimizing or preventing tooth root resorption (e.g., apical root resorption), comprising administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods also comprise allowing a force, in one or more embodiments, a heavy force, to be exerted on one or more teeth of the patient in need thereof, wherein the light is administered before, during, or after the force is exerted. Methods for reducing bone resorption or inflammatory dentin or cementum resorption of the tooth root or periodontium are further provided in accordance with another aspect of the invention. Such methods comprise administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods also comprise allowing a force, in one or more embodiments, a heavy force, to be exerted on one or more teeth of the patient in need thereof, wherein the light is administered before, during, or after the force is exerted.

Another aspect of the invention provides methods for preventing or minimizing inflammation of tissue surrounding one or more teeth upon which forces, force, in one or more embodiments, heavy forces, are or were exerted, comprising administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods also comprise allowing a force, in one or more embodiments, a heavy force, to be exerted on one or more teeth of a patient in need thereof, wherein the light is administered before, during or after the force is exerted.

Another aspect of the invention provides methods for regenerating maxillary or mandibular alveolar bone, comprising administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods also comprise allowing a force, in one or more embodiments, a heavy force, to be exerted on one or more teeth of a patient in need thereof, wherein the light is administered before, during, or after the force is exerted.

In one or more embodiments, the methods further comprise allowing a functional appliance to exert a force on one or more teeth of the patient in need thereof; wherein the functional appliance exerts the force before, during or after the force, in one or more embodiments, the heavy force, is exerted and/or the light is administered. In this manner, the force can be exerted in combination with or in lieu of the force exerted by the functional appliance. In one or more embodiments, the methods comprise further administering an effective amount of vitamin D to the patient. The vitamin D can be administered before, during or after the force is exerted, the functional appliance exerts a force, and/or the light is administered. In this manner, the vitamin D can be administered to the patient in combination with or in lieu of the force being exerted or the functional appliance exerting a force.

Exerting Forces

As indicated herein, in one or more embodiments, a force is allowed to be exerted on one or more teeth of a patient in need thereof. In one or more embodiments, the force is allowed to be exerted on one or more teeth of the patient prior to, subsequent to or during administration of light from an apparatus of the invention. In one or more embodiments, the force can be an orthopedic force. For example, in one or more embodiments, the orthopedic force includes a force applied to one, two, or more teeth sufficient to cause movement in one or more bones underlying the tooth (or teeth). In one or more embodiments, an orthopedic force is a force having a magnitude of greater than about 300 grams of force. In other embodiments, an orthopedic force is a force having a magnitude of greater than or equal to about 350 grams of force, greater than or equal to about 400 grams of force, greater than or equal to about 450 grams of force, greater than or equal to about 500 grams of force, greater than or equal to about 550 grams of force, or greater than or equal to about 600 grams of force. In other embodiments, an orthopedic force is a force having a magnitude of less than or equal to about 500 grams of force, less than or equal to about 550 grams of force, less than or equal to about 600 grams of force, less than or equal to about 650 grams of force, less than or equal to about 700 grams of force, less than or equal to about 800 grams of force, less than or equal to about 900 grams of force, or less than or equal to about 1000 grams of force. In other embodiments, an orthopedic force ranges from about 300 grams of force to about 1000 grams of force. In other embodiments, an orthopedic force's lower range is about 300 grams of force, about 350 grams of force, about 400 grams of force, about 500 grams of force, about 600 grams of force or about 700 grams of force. In other embodiments the orthopedic force's upper range is about 500 grams of force, about 550 grams of force, about 600 grams of force, about 650 grams of force, about 700 grams of force, about 800 grams of force, about 900 grams of force, or about 1000 grams of force. In other embodiments, a force that is less than an orthopedic force is exerted on one or more of a patient's teeth. In this embodiment, the force has a magnitude of less than 100 grams of force, for example, a magnitude of about 200 grams of force or about 300 grams of force.

In one or more embodiments, the force is a less-than-orthopedic force. In one or more embodiments, a less-than-orthopedic force is a force having a magnitude of greater than about 30 grams of force. In other embodiments, a less-than-orthopedic force is a force having a magnitude of greater than or equal to about 50 grams of force, greater than or equal to about 75 grams of force, greater than or equal to about 100 grams of force, greater than or equal to about 150 grams of force, greater than or equal to about 200 grams of force, or greater than or equal to about 250 grams of force. In other embodiments, a less-than-orthopedic force is a force having a magnitude of less than or equal to about 50 grams of force, less than or equal to about 75 grams of force, less than or equal to about 100 grams of force, less than or equal to about 150 grams of force, less than or equal to about 200 grams of force, less than or equal to about 250 grams of force, or less than or equal to about 275 grams of force. In other embodiments, a less-than-orthopedic force ranges from about 30 grams of force to about 300 grams of force. In other embodiments, a less-than-orthopedic force's lower range is about 30 grams of force, about 50 grams of force, about 75 grams of force, about 100 grams of force, about 150 grams of force, about 200 grams of force, or about 250 grams of force. In other embodiments the less-than-orthopedic force's upper range is about 50 grams of force, about 75 grams of force, about 100 grams of force, about 150 grams of force, about 200 grams of force, about 250 grams of force, or about 275 grams of force.

In one or more embodiments, the force is a heavy force. The phrase “heavy force” as used herein refers to a force that ranges from about 150 grams of force to about 1000 grams of force, and that is exerted on a tooth. For example, in one or more embodiments, a heavy force is a force having a magnitude of greater than about 150 grams of force. In other embodiments, a heavy force is a force having a magnitude of greater than or equal to about 175 grams of force, greater than or equal to about 190 grams of force, greater than or equal to about 200 grams of force, greater than or equal to about 210 grams of force, greater than or equal to about 225 grams of force, or greater than or equal to about 250 grams of force. In other embodiments, a heavy force is a force having a magnitude of less than or equal to about 300 grams of force, less than or equal to about 350 grams of force, less than or equal to about 400 grams of force, less than or equal to about 450 grams of force, less than or equal to about 500 grams of force, less than or equal to about 550 grams of force, or less than or equal to about 600 grams of force, and so on up to less than or equal to about 1000 grams of force. In other embodiments, however, a heavy force ranges from about 150 grams of force to about 600 grams of force. In other embodiments, the heavy force's lower range is about 175 grams of force, about 190 grams of force, about 200 grams of force, about 210 grams of force, about 225 grams of force or about 250 grams of force. In other embodiments, the heavy force's upper range is about 300 grams of force, about 350 grams of force, about 400 grams of force, about 450 grams of force, about 500 grams of force, about 550 grams of force, or about 600 grams of force, and so on up to about 1000 grams of force. In one or more embodiments, the heavy force ranges from about 200 grams of force to about 500 grams of force. In other embodiments, the heavy force ranges from about 250 grams of force to about 450 grams of force. In one or more embodiments, the heavy force ranges from about 150 grams of force to about 300 grams of force.

In one or more embodiments, a heavy force is exerted on one or more teeth of the patient. For example, a heavy force can be exerted on one or more of the patient's teeth before, during, or after being administered with an effective amount of light to a region of the patient's gum (e.g., the alveolar soft tissue). In other embodiments, however, a force that is less than a heavy force is exerted on one or more of a patient's teeth. In this embodiment, the force has a magnitude of less than 150 grams of force, for example, a magnitude of about 100 grams of force or about 125 grams of force.

In one or more embodiments, the force exerted on one or more teeth of the patient can be a less-than-heavy force. Such a force can be exerted, for example, by a functional appliance or an orthodontic appliance. In one or more embodiments, a less-than-heavy force is a force having a magnitude of greater than about 10 grams of force. In other embodiments, a less-than-heavy force is a force having a magnitude of greater than or equal to about 20 grams of force, greater than or equal to about 30 grams of force, greater than or equal to about 40 grams of force, greater than or equal to about 50 grams of force, greater than or equal to about 75 grams of force, greater than or equal to about 100 grams of force, or greater than or equal to about 125 grams of force. In other embodiments, a less-than-heavy force is a force having a magnitude of less than or equal to about 30 grams of force, less than or equal to about 40 grams of force, less than or equal to about 50 grams of force, less than or equal to about 75 grams of force, less than or equal to about 100 grams of force, or less than or equal to about 150 grams of force. In other embodiments, a less-than-heavy force ranges from about 10 grams of force to about 150 grams of force. In other embodiments, a less-than-heavy force's lower range is about 10 grams of force, about 20 grams of force, about 30 grams of force, about 50 grams of force, about 75 grams of force, about 100 grams of force, or about 125 grams of force. In other embodiments the less-than-heavy force's upper range is about 30 grams of force, about 40 grams of force, about 50 grams of force, about 75 grams of force, about 100 grams of force, or less than about 150 grams of force.

The phrase “magnitude of force” as used herein refers to the amount of force exerted per tooth. Alternatively, the “magnitude of force” can refer to the amount of force exerted on a plurality of teeth. The magnitude of force exerted per tooth in the latter instance is the total magnitude of force divided by the number of teeth. For example, if about 300 grams of force are exerted on to two teeth, then the force exerted on each tooth is about 150 grams. The phrase “gram of force” as used herein refers to a unit of force equal to the magnitude of force exerted on one gram of mass by a force of 9.80665 m/s2 (i.e., standard gravity). In one or more embodiments, the magnitude of force is a gram of force that is exerted on a tooth. In other embodiments, the magnitude of force is a gram of force that is exerted on a plurality of teeth.

In one or more embodiments, a force is a force of sufficient magnitude to cause at least some amount of tooth-root resorption. In one or more embodiments, an amount of tooth-root resorption caused by a force is correlated to the amount of force exerted such that an increase in the force exerted causes an increase in the amount of tooth-root resorption. In one or more embodiments, a force has sufficient magnitude to have pathophysiological effects, to create a hyalinized zone or tissue death, to cause cell death, or to cause tissue inflammation when the force is exerted without any other form of treatment, such as light treatment. The force can be an excessive pathophysiological force. A pathophysiological force can cause necrosis or root resorption. The force can also cause pressure on the periodontium that can result in ischemia, decreased blood flow, or cell death.

A force can be exerted on a tooth in any suitable manner. For example, in one or more embodiments, the force is exerted normal (e.g., orthogonal or at a 90 degree angle) relative to a side of one or more teeth. In one or more embodiments, the force is exerted at an angle relative to a side of one or more teeth. For example, the force can be exerted at an angle of about 45 degrees, about 60 degrees, about 70 degrees, about 75 degrees, about 80 degrees, about 85 degrees, about 90 degrees, about 95 degrees, about 100 degrees, about 105 degrees, about 110 degrees, about 120 degrees, or about 135 degrees relative to a side of one or more teeth. A force can be exerted normal (e.g., orthogonal or at a 90 degree angle) to, downwards to, or upwards to one or more teeth at any angle. In one or more embodiments, a proximal force is applied to one or more teeth. In some other embodiments, a distal force is applied to one or more tooth. In one or more embodiments, the force is coronal pressure, e.g., a pressure exerted in the direction of or on the crown of the tooth, which is useful to intrude teeth; in other embodiments, the force is apical pressure, e.g., a pressure exerted in the direction of or on the root, which is useful to extrude teeth. In one or more embodiments, a force is exerted on a mesial (e.g., side of tooth towards front of mouth) side of the tooth. In one or more embodiments, a force is exerted on a distal, e.g., side of tooth towards back of mouth) side of the tooth. A force can be exerted on a buccal, e.g., side of tooth towards cheek, side of the tooth, or a force can be exerted on a lingual, e.g., side of tooth towards tongue, side of the tooth. A force can be exerted on an occlusal surface of a tooth. A force can be exerted on an incisal surface of a tooth. A force can be exerted on a proximal surface of a tooth, e.g., mesial or distal surfaces in between teeth. A force can be exerted on an apical, e.g., toward a root end, surface of a tooth. In one or more embodiments, a force exerted on a tooth is translated to be exerted on the mandibular bone or maxillary bone. The force can be exerted by a functional appliance for regulating oral or maxillofacial bone remodeling. In one or more embodiments, the force can be exerted by an orthodontic appliance for regulating tooth movement.

A force can be directed to push one or more teeth toward one another. A force can be directed to push one or more teeth apart. A force can be directed to move one or more teeth toward a side. In one or more embodiments, a force can shift a tooth sideways along a maxilla or mandible. Alternatively, a force can move a tooth forwards or backwards relative to a maxilla or mandible. In one or more embodiments, a force can be directed to move a mandibular bone or maxillary bone forward in an anterior direction. A force can be directed to move a mandibular bone or maxillary bone backward in a posterior direction. A force can be directed to adjust an angle of a mandibular bone or maxillary bone. For example, the angle of a mandibular bone can be adjusted by moving a right side or a left side of a mandibular bone forward or backward. If a right side of a mandibular bone is moved forward or lengthened, while the left side of the mandibular bone maintains the same position or is moved backward or shortened, the mandibular bone can be angled more leftward (e.g., shifted sideways or to the left side). In other words, a force can be directed to move one or more teeth toward a side.

In one or more embodiments, a force is exerted at any point or region along a side of one or more teeth and/or along a side of an oral or maxillofacial bone, muscle, or soft tissue. In one or more embodiments, a force is exerted at or near the top of one or more teeth, i.e., the side of a tooth opposite its root or roots. In one or more embodiments, a force is exerted at or near the middle of the clinical crown, e.g., exposed to the air, above the gums, of one or more teeth. In other embodiments, a force is exerted at or near the bottom of the clinical crown of one or more teeth, i.e., the clinical crown of a tooth closer to its root. In one or more embodiments, the force is applied to the root of the one or more teeth. A force can be exerted on one or more of the points or regions described herein on one or more teeth. In one or more embodiments, a force is exerted along the side of the tooth. Depending on where or for how long the force is exerted, some or no tipped movement can occur to the tooth. Tipped movement is described in more detail herein.

In one or more embodiments, however, a force is exerted at or near a temporomandibular joint, condyle, or glenoid fossa. In one or more embodiments, a force is exerted on one or more of the right temporomandibular joint, right condyle, or right glenoid fossa; one or more of the left temporomandibular joint, left condyle, or left glenoid fossa; or one or more of both right and left temporomandibular joints, both right and left condyles, and both right and left glenoid fossa. In one or more embodiments, the force is exerted on the right temporomandibular joint without being exerted on the left temporomandibular joint, the right condyle without being exerted on the left condyle, the right glenoid fossa without being exerted on the left glenoid fossa, the left temporomandibular joint without being exerted on the right temporomandibular joint, the left condyle without being exerted on the right condyle, or the left glenoid fossa without being exerted on the right glenoid fossa. In one or more embodiments, the force is exerted on mandibular or maxillary alveolar bone. In one or more embodiments, the force is exerted on an anterior portion of the maxillary bone, mandibular bone, or temporal bone.

Depending on where or for how long the force is exerted, some or no tooth tipped movement can occur. In one or more embodiments, a force can increase the velocity of tooth movement as compared to where no force or a lighter force is exerted. In these embodiments, in other words, the force reduces the amount of time it takes for the tooth to move to its desired position within the gum. Exertion of a force on the maxillary bone, mandibular bone, temporal bone, or one or more of a patient's teeth, particularly where the patient is administered with an effective amount of light to his or her maxillary bone, mandibular bone, or one or more teeth, can further reduce the amount of time of orthodontic treatment that a patient might undergo.

In one or more embodiments, a force is exerted on one or more teeth of a patient by one or more orthodontic appliances. Accordingly, in one or more embodiments, an orthodontic appliance can exert a force on one or more of the patient's teeth to facilitate tooth movement. In one or more embodiments, a functional appliance exerts a force on oral or maxillofacial bone, muscle, soft tissue, or one or more teeth. The functional appliance can exert a force on only the mandibular bone of the patient. Alternatively, the functional appliance can exert a force only the maxillary bone of the patient. In one or more embodiments, the functional appliance exerts a force on only the temporal bone of the patient. The functional appliance can exert a force on both the mandibular bone and maxillary bone of the patient. The functional appliance can optionally exert a force on a maxillary bone, mandibular bone, or temporal bone by exerting a force on one or more tooth of the patient. The functional appliance can exert a force on only the jaw muscle. The functional appliance can exert a force on only the jaw soft tissue.

In one or more embodiments, the orthodontic appliance can be present on one or more of the patient's teeth, other oral regions of the patient, or the patient's head or face. In one or more embodiments, the patient wears two or more orthodontic appliances and less than all of these appliances exert a force on one or more of the patient's teeth. For example, the orthodontic appliance can exert a force on only one tooth of the patient or, alternatively, the orthodontic appliance can exert a force on a plurality of teeth of the patient. In another embodiment, the orthodontic appliance can selectively exert a force on less than all the teeth of the patient. Orthodontic appliances that exert forces can also include other intra-oral appliances and/or extra-oral appliances.

In one or more embodiments, the orthodontic appliance for exerting a force can be useful for external anchorage, and can be the form of a temporary anchorage apparatus or in the form of headgear. For example, a patient that uses the intra-oral apparatus can concurrently wear a second orthodontic appliance, e.g., in the form of headgear, for temporary period of time, e.g., at night. In one or more embodiments, the externally worn headgear can physically or electronically communicate with an intra-oral apparatus to facilitate tooth movement. External anchorage can be useful for facilitating the exertion of forces to prevent untoward movement of anchorage teeth during use of forces.

As is described in more detail herein, the patient can wear an orthodontic appliance that exerts forces subsequent to initiating the administration of light. For example, the patient can wear an orthodontic appliance that exerts forces after one or more light treatment sessions are completed while using an intra-oral apparatus. In this manner, a force can be exerted on one or more teeth of the patient by the orthodontic appliance(s) subsequent to initiating the administration of light via an intra-oral apparatus. In one or more embodiments, however, a force is exerted on one or more teeth of the patient prior to or during the administration of light. In such an embodiment, the patient wears an orthodontic appliance and uses an intra-oral apparatus at the same time. In other embodiments, a force is exerted on one or more teeth of the patient prior to and during the administration of light. In one or more embodiments, a force is exerted on one or more teeth of the patient and the intra-oral apparatus emits light during the alignment phase of orthodontic treatment. In another embodiment, the patient uses a single intra-oral apparatus that both administers light and exerts a force. In other embodiments, a force is exerted on one or more teeth of the patient prior to initiating the administration of light. The patient, for example, could wear his or her orthodontic appliance for any length of time before beginning the light treatment.

In some instances, heavy forces can cause a periodontal ligament to compress, which can eventually lead to ischemia or cell death. To prevent ischemia or eventual cell death, the heavy force is exerted with the light treatment as described herein. In one or more embodiments, however, the heavy force is exerted after the light treatment has started. In one or more embodiments, the heavy force is exerted minutes, hours, or days after light treatment has started. In this manner, the light treatment can provide additional adenosine-5′-triphosphate (ATP) energy to tissue cells that will become stressed and could potentially become ischemic as a result of the heavy force. Illustrative frequencies of light treatment used by an intra-oral apparatus are described herein. In one or more embodiments, the heavy force is exerted concurrently with administration of light. In other embodiments, the heavy force is exerted subsequent to administration of light.

As described herein, a heavy force can be exerted on one of more teeth from any direction. More particularly, in one or more embodiments, the force pushes two or more teeth together or apart, or pushes one or more teeth to one side or area of a patient's mouth. For example, in one or more embodiments, the force can push two or more teeth toward the front of the patient's mouth, to the back of the patient's mouth, to the left of the patient's mouth, or to the right of the patient's mouth.

Regulating oral or maxillofacial bone remodeling can comprise changing the position of the mandibular bone or maxillary bone relative to one another or to the skull of the patient. Regulating oral or maxillofacial bone remodeling can also comprise controlling the position (e.g., forward, backward, sideways or angle) of the mandibular bone or maxillary bone, lengthening or shortening the mandibular bone or maxillary bone, lengthening or shortening a side of the mandibular bone or maxillary bone, altering the shape or dimensions of the mandibular bone or maxillary bone, or regulating (e.g., increasing, decreasing or maintaining) the velocity of the movement of the mandibular bone or maxillary bone relative to one another. For example, regulating oral or maxillofacial bone remodeling can comprise increasing the velocity of oral or maxillofacial bone remodeling.

By repositioning a mandibular bone forward or backwards, muscle tension can be caused on the joint area of the mandibular bone, or other parts of the mandibular bone. This tension can stimulate osteoblastic activity or bone remodeling, which can lengthen the mandibular bone through bone deposition on the condylar head and glenoid fossa of the temporal bone of the skull. Also, the tension can effect dental movement forward of the entire lower arch. In some cases, antagonistic force on the maxillary bone can retard the growth of the maxillary bone and cause remodeling and dental movement posteriorly. This can be desirable in situations where the oral or maxillofacial bone remodeling is regulated in order to remodel the maxillary bone posteriorly. Malocclusion can exist when there is a misalignment of teeth or the upper dental arch and the lower dental arch do not line up. The antagonist force on the maxillary bone can be more or less desirable depending on the severity of the malocclusion and whether the maxillary bone is protrusive. If the maxillary bone is protrusive, it can be desirable to retard maxillary forward growth or even retrude maxillary teeth and the jaw bone. A maxillary headgear can be useful to retard or decrease the growth of the maxilla forward. In one example, a functional appliance can be useful for repositioning a mandibular bone forward while utilizing upper teeth or the maxillary bone as anchorage. An equal and opposite force can be exerted on the maxillary bone, which can lead to dental orthodontic movement and bone remodeling on the maxillary bone.

Some functional appliances (e.g., Bionator or Frankel), can prevent antagonist muscles from pushing on the bone and teeth. This can permit opposite agonist muscles to push on the bone and teeth. Thus, in one or more embodiments, allowing a force to be exerted on an oral or maxillofacial bone, muscle, or soft tissue, or one or more teeth, can comprise preventing a first group of muscles from exerting a force on the oral or maxillofacial bone, muscle, or soft tissue, or one or more teeth, thereby allowing a second group of muscles to exert the force. Some examples of muscles whose forces can be withheld, include cheek and lip (peri-oral) muscles. Examples of such muscles can include masseters, buccinators, mentalis muscle and orbicularis. This can allow other muscles, such as the tongue, to exert a force on the oral or maxillofacial bone, muscle, or soft tissue, or one or more teeth. In some cases, it can be desirable to prevent the tongue from interfering with and pushing on teeth, so a functional appliance or an orthodontic appliance can be inserted to prevent the tongue from pushing on the front teeth during swallowing. This could allow cheek and lip muscles to push on teeth and bone to retract and allow teeth to erupt into a normal position previously presented by an overactive and poorly positioned tongue. In one example, a Frankel appliance can hold the cheek and lip muscles away from the teeth to allow them room to grow into the correct position. While the cheek and lip muscles (opposing muscles) are held away from the teeth, the tongue (an agonist muscle pushing against the teeth from the inside) can push on the teeth, thereby allowing a lower arch, upper arch, or both lower and upper arch to expand without interference from the opposing cheek and lip muscles.

In one or more embodiments, the force exerted by a functional appliance can prevent muscles of a first group from exerting a first force, or can substantially reduce the amount of the first force (e.g., by 25% or more), allowing muscles in a second group to exert a second force, which can result in bone remodeling caused by the second force. The muscles in the first group and the muscles in the second group can typically exert forces in different directions. For example, muscles can exert forces anteriorly, posteriorly, laterally to the left, laterally to the right, radially inward, radially outward, upward, or downward. In one or more embodiments, the muscles of the first group and the muscles of the second group can exert forces in a substantially opposite direction (e.g., in directions different by about 180 degrees). The muscles in the first group and the muscles in the second group can exert forces in different directions. Alternatively, the force exerted by the functional appliance can alter the angle of the overall force applied to the region by increasing the relative effect of the second force, which can result in bone remodeling caused by the increased magnitude on the second force relative to the first force. Any number of muscle groups (e.g., 1, 2, 3, 4, 5, 6, or more) can exert force in any direction. The force exerted by the functional appliance can prevent one or more of the muscle groups from exerting a force or can reduce the amount of force exerted by one or more groups.

In one or more embodiments, a functional appliance can keep muscles away from the teeth so that the muscles that oppose those that are withdrawn via the functional appliance then can exert forces on the teeth to cause tooth movement and possible bone remodeling due to “imbalance” of previously balanced muscular pressure. In one or more embodiments, the functional appliance exerts a force on the oral or maxillofacial muscle or soft tissue in order to keep the muscles away.

The phrase “regulating tooth movement” as used herein refers to and includes one or more of the following functions and/or operations. For example, regulating tooth movement can comprise regulating, in one or more embodiments, aligning, the position of one or more teeth relative to a supporting tissue. Regulating tooth movement can also comprise increasing, decreasing or maintaining the velocity of tooth movement relative to a supporting tissue. For example, regulating tooth movement can comprise increasing the velocity, or speed, of tooth movement. Regulating tooth movement can also comprise increasing, decreasing or maintaining the degree of bodily movement, e.g., relative to the degree of tipped movement, of one or more teeth. Regulating tooth movement can comprise moving one or more teeth bodily. “Bodily” movement means generally perpendicular tooth movement relative to the supporting tissue. “Tipped” movement means that the crown or coronal region of the tooth advances more quickly than the root or apical region of the tooth. Bodily tooth movement can occur without causing significant tipped movement of the tooth. By “significant tipped movement” is meant that about 20% of the tooth does not move in the same lateral direction as the remaining about 80%; in another embodiment about 10% of the tooth does not move in the same lateral direction as the remaining about 90%: in another embodiment about 5% of the tooth does not move in the same lateral direction as the remaining about 95%. Tooth movement can include lateral displacement, rotation, extrusion or intrusion of one or more teeth. Regulating tooth movement can comprise inducing the tilting or tipped movement of one or more teeth, minimizing or preventing the tilting or tipped movement one or more teeth, or maintaining or inducing alignment or orientation of the one or more teeth. Regulating tooth movement can also comprise stabilizing, retarding the rate of or preventing tooth movement. In some instances, regulating tooth movement can comprise causing one or more teeth to maintain their position. In one or more embodiments, regulating tooth movement can comprise causing one or both of (i) the displacement of one or more teeth and (ii) the maintenance of one or more other teeth in their position. In one or more embodiments, regulating tooth movement occurs prior to, subsequent to or during orthodontic treatment with an orthodontic appliance. In one or more embodiments, regulating tooth movement occurs prior to, subsequent to or during the alignment phase of orthodontic treatment. In one or more embodiments, tooth movement occurs during bone remodeling.

In one or more embodiments, regulating tooth movement occurs during an alignment phase of orthodontic treatment. Tooth movement during this phase can include the tipping movement of one or more teeth, the rotation of one or more teeth, and/or the extrusion or intrusion of one or more teeth. The extrusion or intrusion of one or more teeth can be a bodily movement that occurs during the alignment phase. In general, however, bodily movement does not usually occur during the alignment phase of orthodontic treatment. Rather, bodily movement typically occurs after the alignment phase of orthodontic treatment, when the teeth are aligned and crowding of the teeth is minimized. In one or more embodiments, regulating tooth movement occurs after the alignment phase of orthodontic treatment. In other embodiments, regulating tooth movement occurs prior to the alignment phase of orthodontic treatment.

In one or more embodiments, the type or shape of wire used in an orthodontic appliance can assist in regulating tooth movement. For example, an orthodontic appliance comprising a round wire worn by a patient during an alignment phase of orthodontic treatment can exert a force that increases the velocity of tipping movement, rotation, extrusion or intrusion of one or more teeth. An orthodontic appliance comprising a rectangular wire is generally worn after the alignment phase, e.g., during the finishing or detailing phase and/or the retention phase, or when the one or more teeth of the patient are aligned from zero (0) mm to less than 1 mm in the same horizontal plane, such that crowding is minimal. A rectangular wire is generally stiffer than a round wire and can facilitate the bodily movement of one or more teeth.

In one or more embodiments, tooth movement is measured by a change in Little's Irregularity Index (LII). LII measures the degree of discrepancy between teeth and is the sum of the five (5) linear distances from one contact point to an adjacent contact point of the six (6) anterior teeth. An LII score of zero (0) indicates that the teeth are perfectly aligned. In one or more embodiments, one or more teeth of one or both of the upper arch or the lower arch are moved into alignment to the extent that the one or both of the upper-arch LII or the lower-arch LII ranges from zero (0) mm to less than 1 mm. An LII score of 1 mm or greater indicates that the teeth are misaligned and that tooth movement is needed for correction. A contact point measurement of 0 (zero) mm indicates perfect alignment of the two adjacent teeth. A contact point measure of, for example, 0.22 mm indicates that there is a discrepancy of 0.22 mm between the two adjacent teeth. In general, the higher the LII score, the less aligned are the teeth. For example, an LII score greater than 10 mm general indicates a severe misalignment. Additional details regarding LII and its calculations can be found in the following publication, which is incorporated by reference herein in its entirety: Little, R. M., “The irregularity index: a quantitative score of mandibular anterior alignment,” Am. J. Orthod., 1975 November, 68(5): 554-63. The contact point measurements can be taken using a fine-tip digital caliper, such as a Tresna® Point Digital Caliper, Series SC02 commercially available from Guanglu Measuring Instrument Co., Ltd. of China. The caliper can be configured to measure the contact point to the nearest 0.1 mm. Contact point measurements can be taken using models of the patient's teeth.

Orthodontic Appliances

The present methods can be performed on a patient prior to being applied with one or more functional appliances and/or orthodontic appliances, during a time when the patient wears one or more functional appliances and/or orthodontic appliances, or after one or more functional appliances and/or orthodontic appliances has been removed from the patient. The one or more functional appliances and/or orthodontic appliances can be used in addition to the intra-oral apparatus (e.g., any light therapy apparatus described herein, including, but not limited to, light therapy apparatuses 2500 and 3500). A functional appliance or orthodontic appliance can be fixed or movable with respect to a patient's teeth and/or the intra-oral apparatus. As is disclosed herein, orthodontic appliances can include, for example, fixed active appliances such as pin and tube appliances, appliances using wires or brackets or springs, ribbon arch appliances. Begg lightwire appliances, edgewise appliances, pre-adjusted edgewise appliances, self-ligating edgewise appliances, bi-helix, tri-helix, quad-helix, rapid maxillary expansion appliance (RME); removable active appliances such as expansion and labial segment alignment appliance INVISALIGN™; substantially transparent aligners: orthodontic headgear including reverse headgear and conventional headgear, and other types of orthodontic apparatus. In one or more embodiments the orthodontic appliance comprises wires and brackets (examples of which are described herein, with respect to the section entitled “Examples”).

In one or more embodiments, the orthodontic appliance is fixed. Examples of fixed orthodontic appliances include pin and tube appliances, ribbon arch appliances, Begg Lightwire appliances, edgewise appliances, pre-adjusted edgewise appliances, self-ligating edgewise appliances, hi-helix appliances, tri-helix appliances, quad helix appliances, rapid maxillary expansion appliances (RME), or pin stripe appliances. Fixed orthodontic appliances can be fixed to the patient's teeth during orthodontic treatment. In one or more embodiments, the orthodontic appliance is fixed and comprises wires and brackets. In another embodiment, the orthodontic appliance is removable. Examples of removable orthodontic appliances include Active Hawley appliances, INVISALIGN™ aligners, aligners, fan expanders, or sagittal appliances.

In one or more embodiments, the functional appliance is a mandibular repositioner or any other intra-oral apparatus that repositions the mandible to create tension on tissue to stimulate bone remodeling or tooth movement. Some examples of mandibular repositioners are Herbst, Twin Block, Fixed Twin Block, Bonded Twin Block, Biobloc, Forsus Fatigue (e.g., EZ2), Xbow (Crossbow), mandibular anterior repositioning appliance (Mara), Bass Dynamax, Bionator, Open Face Activator, Activator, Woodside Activator, Frankel (e.g., Frankel I, II, III, IV, V), Teuscher appliance, Andreson appliance, 3-Way Sagittal, Lower Schwartz, 3 Way Expander, jaw repositioning appliances, removable orthotic appliances, Series 2000®, BioPedic Appliance. Rick-A-Nator™, Ritto Appliance, Eureka Spring™, Twin Force Bite Corrector™, Alpern Class II Closers, Rapid palatal expander. Tandem™, facemask, reverse pull headgear, and conventional orthodontic headgear.

In one or more embodiments, the functional appliance is fixed. A fixed functional appliance can be cemented, for example, on one or more teeth. Some examples of fixed functional appliances include Herbst, Fixed Twin Block, Bonded Twin Block. Forsus Fatigue (e.g., EZ2), Xbow (Crossbow), Series 2000®, BioPedic Appliance, Rick-A-Nator™, Ritto Appliance, Eureka Spring™, Twin Force Bite Corrector™, Alpern Class II Closers, and Rapid palatal expander. In another embodiment, the functional appliance is removable. Some examples of removable functional appliances include Twin Block, Biobloc, mandibular anterior repositioning appliance (Mara), Bass Dynamax, Bionator, Open Face Activator, Activator, Woodside Activator, Frankel (e.g., Frankel I, II, III, IV, V), Teuscher appliance, Andreson appliance, 3-Way Sagittal, Lower Schwartz, 3 Way Expander, jaw repositioning appliances, and removable orthotic appliances. In one or more embodiments, the functional appliance is a combination fixed-removable functional appliance. A combination fixed-removable functional appliance can comprise one or more components that are fixed to a patient's teeth and one or more component that is removable from the fixed component. Some examples of combination fixed-removable functional appliances include Tandem™, a facemask, reverse pull headgear, and conventional orthodontic headgear.

In one or more embodiments, the functional appliance is a Class II corrector. Some examples of Class II correctors include Herbst, Twin Block, Forsus Fatigue, and Mara. In other embodiments, the functional appliance is a Class I corrector that is useful for creating and bony and dental expansion of crowded and lower arches. In other embodiments, the functional appliance is a Class III corrector that is useful for stimulating maxillary forward growth, or retruding or limiting mandibular growth.

In one or more embodiments, the functional appliances reposition a patient's mandibular bone anteriorly. The functional appliance can be a fixed functional mandibular repositioner. Examples of such functional appliances are a Herbst, Twin Block, Bonded Twin Block, Biobloc, and Bass Dynamax. In one or more embodiments, the functional appliances expand the jaw (e.g., using muscular pressure or lack of muscular forces to allow teeth to move and/or bone to remodel). Examples of such functional appliances can include Bionator, Open Face Activator, Activator, Woodside Activator, or Frankel. Light can be administered to the alveolar soft tissue and/or alveolar bones and teeth, as these appliances can cause orthodontic movement of teeth as well as bone remodeling. In one or more embodiments, the functional appliances control growth of the maxillary bone or mandibular bone. Examples of such functional appliances can include a facemask, or reverse pull headgear. Light can be administered to apical areas of the jaw, which can cause some orthodontic movement, but primarily remodels and provides anterior movement of maxillary bone. In one or more embodiments, the functional appliances exert a force on, or cause bone remodeling at, a temporomandibular joint, condyle, or glenoid fossa of a patient.

A functional appliance functions by exerting a force that causes muscle or tissue to exert a force directly on, for example, a tooth such that some aspect of the tooth changes as a result of said force from the muscle or tissue. In one specific example, a patient can wear a functional appliance to reposition his or her jaw, and the resultant position of the jaw exerts a force on surrounding tissue thereby allowing remodeling to occur. Functional changes can include changes in the maxillary bone, the mandibular bone, tooth position, bite and jaw function, and chewing. In contrast to functional appliances, orthodontic appliances function by exerting a force directly on, for example, a tooth to change some aspect of the tooth (e.g., to change the position of the tooth relative to another tooth).

Orthodontic appliances are commercially available and can include specifications (or other documentation) that specify the magnitude of force that the appliance is capable of exerting on one or more teeth. In one or more embodiments, an orthodontic appliance comprises steel wires, nickel titanium wires, or titanium molybdenum wires. In one or more embodiments, an orthodontic appliance comprises wires or springs that are of a high gauge. Some examples of wires that an orthodontic appliance can comprise are stainless steel or nickel-titanium wires having wire dimensions of one of the following:

| 0.0160″ square | |
|---|---|
| 0.406 mm square | |
| 0.0160″ × 0.0220″ | 0.406 mm × 0.559 mm |
| 0.0170″ square | 0.432 mm square |
| 0.0170″ × 0.0220″ | 0.432 mm × 0.559 mm |
| 0.0170″ × 0.0250″ | 0.432 mm × 0.635 mm |
| 0.0180″ square | 0.457 mm square |
| 0.0180″ × 0.0220″ | 0.457 mm × 0.559 mm |
| 0.0180″ × 0.0250″ | 0.457 mm × 0.635 mm |
| 0.0190″ square | 0.483 mm square |
| 0.0190″ × 0.0250″ | 0.483 mm × 0.635 mm |
| 0.0200″ square | 0.508 mm square |
| 0.0210″ × 0.0250″ | 0.533 mm × 0.635 mm |

A practitioner installing the wires on a patient's teeth can select the appropriate wire(s) for the patient's orthodontic treatment program. In one or more embodiments, a first wire with a first dimension and/or first strength is installed at the time brackets are initially bonded to the patient's teeth, and a second wire with a second dimension and/or second strength is installed a time subsequent to bonding of the brackets, such as during a follow-up visit to the practitioner and, for example, after one or more light therapy treatment session have been administered to the patient's teeth with the orthodontic appliance installed thereon.

An orthodontic appliance can comprise brackets and wires. Commercially available brackets include those offered by SPEED System (www.speedsystem.com), DENTSPLY GAC International (www.gacinovation.com), brackets offered by Ormco Corporation (www.ormco.com) (e.g., Mini-Diamond® brackets), In-Ovation L Straightwire system brackets, or brackets offered by Orthodontic Design and Production, Inc. (e.g., Agility® self-ligating brackets). Wires can be nickel titanium and can have a diameter of 0.012 inch, 0.014 inch or 0.016 inch. In one or more embodiments, the wires are square or rectangular. In one or more embodiments, the wires are square and have a dimension of 0.015 inch×0.015 inch. In one or more embodiments, the wires have a dimension of 0.016 inch×0.016 inch. In another embodiment, the wire is rectangular and have a dimension of 0.017 inch×0.025 inch. In one or more embodiments, the wires are 0.016 inch Supereable nickel titanium wires.

Nickel-titanium closed or open-coil springs can be used. Some examples can include an elastomeric power chain, which can be capable of providing 100-800 grams of force, or intra-arch elastics. In one or more embodiments, the orthodontic appliance comprises an elastic material. An orthodontic appliance can exert a force on one or more teeth of the patient in addition to or in lieu of the intra-oral apparatus exerting a force on one or more teeth. For example, in one or more embodiments, the orthodontic appliance can exert or be configured to exert a heavy force on one or more teeth of the patient in addition to or in lieu of the intra-oral apparatus exerting a heavy force on one or more teeth. The orthodontic appliance can cause one or more teeth to move or maintain its position. In one or more embodiments, an orthodontic appliance causes bone remodeling of an oral or maxillofacial bone, or one or more tooth, such as a mandibular bone, maxillary bone, or temporal bone. In one or more embodiments, an apparatus of the invention does not exert a force on a patient's teeth. In one or more embodiments, an apparatus of the invention does not exert a heavy force on a patient's teeth.

A force, such as a heavy force, can be measured using a dynamometer or any similar device. For example, a dynamometer can measure the force that a wire, spring or similar mechanism from an orthodontic appliance exerts on one or more teeth or gums. In one example, the dynamometer (or similar device) can measure the force that the wires 12 from the intra-oral apparatus depicted in FIG. 3A exert on one or more teeth or gums. The measured force can depend on any number of parameters such as, for example, the gauge of the wire or the stiffness of the wire. In this manner, in one or more embodiments, a force can be calculated, in part, by measuring the tension or stiffness of the appliance's wire (or spring or similar mechanism), e.g., when such force is exerted on one or more teeth. Furthermore, in one or more embodiments, the appliance's wire (or spring or similar mechanism) is constructed from a material that is sensitive to temperature such that the stiffness of the wire, and therefore the heavy force exerted by that wire, can change based on the temperature of the wire. For example, in one or more embodiments, the stiffness of the wire (or spring or similar mechanism) increases when the wire temperature increases, and decreases when the wire temperature decreases. Thus, in some such embodiments, a force can be calculated, in part, by measuring the temperature of the wire (or spring or similar mechanism) or estimating its temperature when present in a patient's oral cavity. With respect to the gauge of the wire, it is generally well known in the art that increasing the gauge (or cross-section) of a wire can increase the stiffness of the wire which ultimately increases the heavy force that the wire exerts on one or more teeth.

Although the methods are described herein as being performable on a patient (1) prior to the patient being applied with one or more functional appliances and/or orthodontic appliances, (2) during a time when the patient wears one or more functional appliances and/or orthodontic appliances, or (3) after one or more functional appliances and/or orthodontic appliances has been removed from the patient, in one or more embodiments, the methods described herein can be performed on the patient independently of or without usage of a functional appliance and/or orthodontic appliance.

In one or more embodiments, a patient can use an intra-oral apparatus of the invention prior to, subsequent to or during a time that the patient wears an orthodontic appliance. In one or more embodiments, the patient begins use of the intra-oral apparatus on the same date the orthodontic appliance is installed on the patient's teeth. The orthodontic appliance installation can include bonding brackets to the patient's teeth and installing wire with the brackets. In other embodiments, the patient discontinues use of the intra-oral apparatus no more than about one or more days (e.g., one, two, or three days) or one or more weeks (e.g., one or two weeks) before or after the orthodontic appliance is installed. In one or more embodiments, the patient discontinues use of the intra-oral apparatus on the same day that the orthodontic appliance is uninstalled from the patient's teeth. In other embodiments, the patient discontinues use of the intra-oral apparatus no more than about one or more days (e.g., one, two, or three days) or one or more weeks (e.g., one or two weeks) before or after the orthodontic appliance is uninstalled.

In one or more embodiments the invention provides methods for regulating tooth movement, maintaining oral tissue health or improving oral tissue health, comprising administering to a patient in need thereof an effective amount of light from the emitter of an intra-oral apparatus of the invention. In one or more embodiments, at least a portion of the apparatus is configured to contact the patient's alveolar soft tissue. In one or more embodiments, the patient wears an orthodontic appliance that exerts a force on one or more teeth of the patient. In one or more embodiments, the light is administered during the alignment phase of orthodontic treatment. In one or more embodiments, the light is administered during only the alignment phase of orthodontic treatment. In one or more embodiments, the alveolar soft tissue is alveolar mucosa. In one or more embodiments, the force is a heavy force. In one or more embodiments, the patient wears the same or a different orthodontic appliance that exerts a force on one or more teeth of the patient during the retention phase of orthodontic treatment. In one or more embodiments, the force exerted during the retention phase is a heavy force.

In one or more embodiments the invention provides methods for orthodontic treatment, comprising administering to a patient in need thereof an effective amount of light from the emitter of an intra-oral apparatus of the invention. In one or more embodiments, at least a portion of the apparatus is configured to contact the patient's alveolar soft tissue. In one or more embodiments, the patient wears an orthodontic appliance that exerts a force on one or more teeth of the patient. In one or more embodiments, the light is administered during the space or gap closure phase of orthodontic treatment. In one or more embodiments, the light is administered during the alignment phase of orthodontic treatment. In one or more embodiments, the light is administered during only the alignment phase of orthodontic treatment. In one or more embodiments, the alveolar soft tissue is alveolar mucosa. In one or more embodiments, the force is effective for regulating tooth movement, maintaining oral tissue health or improving oral tissue health. In one or more embodiments, the force is a heavy force. In one or more embodiments, the patient wears the same or a different orthodontic appliance that exerts a force on one or more teeth of the patient during the retention phase of orthodontic treatment. In one or more embodiments, the force exerted during the retention phase is a heavy force.

In one or more embodiments the invention provides methods for orthodontic treatment, comprising administering to a patient who wears an orthodontic appliance or is in need of orthodontic treatment an effective amount of light from the emitter of an intra-oral apparatus of the invention (e.g., light therapy apparatus 2500 or 3500). In one or more embodiments, at least a portion of the apparatus is configured to contact the patient's alveolar soft tissue. In one or more embodiments, the patient wears an orthodontic appliance that exerts a force on one or more teeth of the patient. In one or more embodiments, the light is administered during the alignment phase of orthodontic treatment. In one or more embodiments, the light is administered during only the alignment phase of orthodontic treatment. In one or more embodiments, the alveolar soft tissue is alveolar mucosa. In one or more embodiments, the force is effective for regulating tooth movement, maintaining oral tissue health or improving oral tissue health. In one or more embodiments, the force is a heavy force. In one or more embodiments, the patient wears the same or a different orthodontic appliance that exerts a force on one or more teeth of the patient during the retention phase of orthodontic treatment. In one or more embodiments, the force exerted during the retention phase is a heavy force.

In one or more embodiments the invention provides methods for orthodontic treatment, comprising administering to a patient who wears an orthodontic appliance and is in need of orthodontic treatment an effective amount of light from the emitter of an intra-oral apparatus of the invention. In one or more embodiments, at least a portion of the apparatus is configured to contact the patient's alveolar soft tissue. In one or more embodiments, the orthodontic appliance exerts a force on one or more teeth of the patient. In one or more embodiments, the light is administered during the space or gap closure phase of orthodontic treatment. In one or more embodiments, the light is administered during the alignment phase of orthodontic treatment. In one or more embodiments, the light is administered during only the alignment phase of orthodontic treatment. In one or more embodiments, the alveolar soft tissue is alveolar mucosa. In one or more embodiments, the force is effective for regulating tooth movement, maintaining oral tissue health or improving oral tissue health. In one or more embodiments, the force is a heavy force. In one or more embodiments, the patient wears the same or a different orthodontic appliance that exerts a force on one or more teeth of the patient during the retention phase of orthodontic treatment. In one or more embodiments, the force exerted during the retention phase is a heavy force.

Vitamin D

As described herein, the present methods can further comprise administering vitamin D to the patient. Vitamin D is essential for normal bone metabolism—it promotes calcium absorption and bone resorption and maintains the necessary calcium and phosphate levels for bone formation. Patients deficient in vitamin D have an increased risk of bone loss and bone fracture, among many other risks. Insufficient vitamin D levels can also interfere with osteoclastic activity, which is essential to tooth movement, resulting in slower tooth movement. Thus, administering vitamin D can be an important part of orthodontic treatment.

The vitamin D can be, for example, vitamin D1, D2, D3, D4, D5, 1,25-dihydroxycholecalciferol, or mixtures thereof. In one or more embodiments, the vitamin D supplements other vitamin D sources for the patient.

The vitamin D can be administered in any suitable manner. For example, the vitamin D can be administered orally, via transdermal gel, by a patch, by a cream, by injection, by electrophoresis, or by insolation. Where the present methods further comprise administering vitamin D, in one or more embodiments, the vitamin D is not administered by insolation. In one or more embodiments, the vitamin D is administered via a vitamin D conveyance. For example, the vitamin D can be present in a composition suitable for oral administration, for example, a pill, capsule, tablet, chewable, gel, or liquid. In other embodiments, the vitamin D is administered transdermally. In one example, the vitamin D can be administered transdermally via a transdermal gel, cream, ointment, liquid, or paste that can be applied to the skin, gums, or any soft tissue. In another example, vitamin D can be administered transdermally via insolation, such as exposure to ultraviolet (UV) rays from the sun or artificially through tanning beds. The vitamin D can also be administered transdermally via a patch or microneedle on the skin, gums, or other soft tissue of the patient. In one or more embodiments, the vitamin D is be administered by injection using a syringe or needle at the skin, gums, or other soft tissue (such as, for example, oral tissue) of the patient. The injection can be intradermal, subcutaneous, intramuscular, intravenous, intraosseous, or intraperitoneal. In one or more embodiments, the vitamin D is administered electrophoretically. The vitamin D can be applied, for example, to the surface of the skin, gums, or any other soft tissue, and a weak electrical current can drive the compound through the tissue.

Any combination of the various vitamin D administration techniques described herein can be employed. For example, a patient can be orally administered with vitamin D and also receive an injection of vitamin D as part of the administration process. In one or more embodiments, the administered vitamin D increases or maintains the vitamin D blood serum levels. In other embodiments, the administered vitamin D increases or maintains local vitamin D levels where the vitamin D is administered.

In one or more embodiments, the vitamin D is administered to a region, or in the proximity of a region. The region can be, for example, an oral region. The region can be, for example, on or in the proximity of oral or maxillofacial bone, muscle, or soft tissue. The region can be on or in the proximity of one or more tooth, the mandibular bone, the maxillary bone, or the temporal bone. In one or more embodiments, the vitamin D is orally administered, for example, via an oral composition that comprises vitamin D. In other embodiments, the vitamin D is administered locally to a region. The region can be on the skin of the patient overlying the patient's face, jawbone, lips, cheek, or chin. The region can be on the right side, the left side, a central region, or any combination thereof, of the patient's body such as, for example, the patient's face. The region can be within the patient's oral cavity. For example, the region can be the gums of the patient, or any other oral soft tissue. The region need not be an oral region; rather, the region can be, for example, on the neck, arm, leg, or torso of the patient. In one or more embodiments, the vitamin D can be administered systemically to the patient. For example, the vitamin D can be administered via insolation through a tanning bed that surrounds the patient's body. The region can include any area previously described.

In one or more embodiments, the vitamin D is administered to a region that is the same as or in the proximity of a region that is administered with light. In one or more embodiments, the vitamin D is administered to the same region that is administered with light. In some other embodiments, the vitamin D is administered to a region having the same, greater, or smaller size than the region administered with light. The vitamin D can be administered to a region adjacent to a region administered with light. In one or more embodiments, vitamin D is administered to a region no more than about 1 mm, about 2 mm, about 3 mm, about 5 mm, about 7 mm, about 10 mm, about 15 mm, about 20 mm, about 25 mm, about 30 mm, about 40 mm, about 50 mm, about 60 mm, about 70 mm, about 10 cm, about 15 cm, about 20 cm, about 30 cm or about 50 cm from a region that is administered with light. In other embodiments, the vitamin D is administered to a region that is different from the region that is administered with light. In one or more embodiments vitamin D is not administered to a region that is administered with light. In one or more embodiments, vitamin D is administered to a region other than the region that is administered with light. In one or more embodiments, vitamin D is administered systemically, which can encompass the region administered with light. In some instances, the vitamin D is administered systemically, raising overall vitamin D levels, which can include vitamin D levels in the region administered with light.

In one or more embodiments, the vitamin D is administered to a region that is proximate to a region upon which a force is exerted. The force can be, for example, a heavy force, a force exerted by an orthodontic appliance, or a force exerted by a functional appliance. In one or more embodiments, the vitamin D is administered to the same region upon which a force is exerted. In one or more embodiments, the region where the vitamin D is administered and the region upon which the force is exerted are the same size. In other embodiments, however, the size of the region where the vitamin D is administered is different from the size of the region upon which the force is exerted. The region where the vitamin D is administered can be, for example, smaller or larger than the region upon which the force is exerted. In one or more embodiments, the vitamin D is administered to a region adjacent to a region upon which a force is exerted. The vitamin D can be administered to a region, for example, nor more than about 1 mm, about 2 mm, about 3 mm, about 5 mm, about 7 mm, about 10 mm, about 15 mm, about 20 mm, about 25 mm, about 30 mm, about 40 mm, about 50 mm, about 60 mm, about 70 mm, about 10 cm, about 15 cm, about 20 cm, about 30 cm or about 50 cm from a region upon which a force is exerted.

In one or more embodiments, the vitamin D is administered to a region that is different from the region upon which a force is exerted. In other words, the vitamin D is not administered to a region upon which a force is exerted. In one or more embodiments, vitamin D is administered systemically and can encompass the region upon which a force is exerted. For example, in some instances, the vitamin D is administered systemically and raises overall vitamin D levels, including the vitamin D levels in the region upon which a force is exerted.

The present methods can comprise administering an effective amount of vitamin D to a patient in need thereof, and administering an effective amount of light to, for example, the alveolar soft tissue, or any other oral or maxillofacial bone, muscle, or soft tissue, or one or more teeth of the patient. In one or more embodiments, the effective amount of vitamin D is administered to an oral region of the patient. Alternatively, the effective amount of vitamin D can be administered systemically to the patient. In one or more embodiments, the method further comprises testing the patient to determine his or her vitamin D level. For example, the patient can undergo blood testing to determine the patient's vitamin D level. If necessary, a patient can receive a vitamin D supplement or treatment. Light can be administered to the alveolar soft tissue and/or teeth in combination with orthodontic treatment and normal or higher vitamin D levels, which can accelerate orthodontic tooth movement.

The present methods can comprise administering an effective amount of vitamin D to a patient and providing any intra-oral light therapy apparatus described herein with reference to FIGS. 1-8. The method can optionally comprise determining whether the patient is vitamin D deficient. The method can optionally comprise measuring the patient's vitamin D blood serum level. In one or more embodiments, if the patient's vitamin D blood serum level is below a predetermined threshold, the patient can administer or be administered with a dosage of vitamin D. In one or more embodiments, the dosage of vitamin D is determined based on the patient's blood serum level and administered to the patient. The dosage of vitamin D to be administered to the patient can be determined, for example, based on the patient's blood serum level, so that the patient is administered with an effective amount of vitamin D. For example, if the patient is very deficient in vitamin D (i.e., has very low vitamin D blood serum levels), the patient can receive a greater dosage of vitamin D than if the patient is only slightly deficient in vitamin D (i.e., has higher vitamin D blood serum levels). In other embodiments, regardless of the vitamin D blood serum level, if the patient is vitamin D deficient, the patient receives the same vitamin D dosage. In yet other embodiments, a dosage of vitamin D is administered to the patient even if the patient is not vitamin D deficient. In embodiments where the patient is vitamin D deficient, the length of vitamin D treatment can vary depending on the degree of vitamin D deficiency.

The vitamin D can be administered in one or more dosages. In one or more embodiments, as described herein, a dosage of vitamin D is an effective amount of vitamin D. In other embodiments, a single dosage of vitamin D can be greater than or less than an effective amount of vitamin D. A dosage of vitamin D can be provided for a period of time. For example, the vitamin D can be administered daily. In one or more embodiments, the vitamin D is administered every hour, several times a day, once a day, once every several days, once a week, once every few weeks, once a month, once every few months, once a quarter, or with any other frequency. Vitamin D can be administered on a regular basis (e.g., every 6 hours, every day, every 10 days), or can be provided at irregular intervals (e.g., twice one day, skip a day, once the next day). In one or more embodiments, vitamin D is administered on an as-needed basis.

In one or more embodiments, the dosage is greater than about, is less than about, or is about 100 IU, about 200 IU, about 400 IU, about 500 IU, about 600 IU, about 800 IU, about 1,000 IU, about 1,200 IU, about 1,500 IU, about 1,600 IU, about 2,000 IU, about 2,500 IU, about 3,000 IU, about 4,000 IU, about 5,000 IU, about 6,000 IU, about 7,000 IU, about 8,000 IU, about 9,000 IU, about 10,000 IU, about 12,000 IU, about 15,000 IU, about 17,000 IU, about 20,000 IU, about 25,000 IU, about 30,000 IU, about 40,000 IU, about 50,000 IU, about 70,000 IU, about 100,000 IU, about 150,000 IU, about 200,000 IU, about 300,000 IU, about 400,000 IU, about 500,000 IU, about 600,000 IU, or about 800,000 IU. In one or more embodiments, the dosage amount varies each time the vitamin D is administered to the patient. In other embodiments, the dosage amount is a daily amount of vitamin D administered to the patient. In other embodiments, the dosage amount is the total vitamin D amount administered for a treatment regimen. For example, a daily oral dosage of vitamin D can range from 400 IU to 6,000 IU per day. In another example, a daily oral dosage of vitamin D can range from 2,000 IU to 6,000 IU per day. The dosage of vitamin D can be a single dose of 600,000 IU of oral vitamin D. Based on one clinical trial, a single dose of 600,000 IU of oral vitamin D was comparable to a dose of 20,000 IU per day of oral vitamin D for 30 days. In another embodiment, the dosage is 20,000 IU per day of oral vitamin D for 30 days.

The dosage of vitamin D can be sufficient to raise the vitamin D blood level from about 40 to about 60 ng/mL of venous blood. The dosage of vitamin D can be sufficient to raise vitamin D blood level to at least about, no more than about, or to about 20 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, or about 80 ng/mL. In one or more embodiments, the dosage of vitamin D is sufficient to raise the vitamin D blood level by any amount. For example, the dosage of vitamin D can be sufficient to raise the vitamin D blood level by about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, or about 60 ng/mL. The vitamin D blood level can be raised to a desired level or by a desired amount within a period of time. For example, the period of time can be no more than about one or more days, one or more weeks, one or more months, or one or more years. For example, a dosage of vitamin D administered daily can raise vitamin D blood serum levels to a desired level no more than about 30 days, or no more than about 3 months following administration of the dosage.

Vitamin D can be administered to the patient prior to, concurrently with, or subsequent to administering light therapy to the patient. Vitamin D can be administered to the patient prior to initiation of the light therapy administration, or prior to the completion of the light therapy administration. In one or more embodiments, a dosage of vitamin D is administered at a period of time (e.g., seconds, minutes, hours, days, weeks, months) prior to initiation of the light therapy administration or prior to completion of the light therapy administration. In one or more embodiments, a dosage of vitamin D is administered at a period of time (e.g., seconds, minutes, hours, days, weeks, months) subsequent to initiation of the light therapy administration or subsequent to completion of the light therapy administration. In one or more embodiments, a vitamin D treatment regimen (which can span one or more doses of vitamin D) is initiated or completed prior to initiation of light therapy administration or prior to completion of light therapy administration. In other embodiments, the vitamin D treatment regimen is initiated or completed subsequent to the initiation of light therapy administration or subsequent to completion of light therapy administration. The vitamin D treatment regimen can be in progress during light therapy administration.

Vitamin D can be administered to the patient prior to, currently with, or subsequent to engaging an intra-oral light therapy apparatus with the patient. The intra-oral light therapy apparatus can be any of the apparatus depicted in FIGS. 1-8. Vitamin D can also be administered to the patient prior to removing the intra-oral light therapy apparatus from the patient. In one or more embodiments, a dosage of vitamin D can be administered at a period of time (e.g., seconds, minutes, hours, days, weeks, months) prior to engaging the intra-oral light therapy apparatus with the patient or prior to removing the intra-oral light therapy apparatus from the patient. In one or more embodiments, a dosage of vitamin D is administered at a period of time (e.g., seconds, minutes, hours, days, weeks, months) subsequent to engaging the intra-oral light therapy apparatus with the patient or subsequent to removing the intra-oral light therapy apparatus from the patient. In one or more embodiments, a vitamin D treatment regimen (which can span one or more doses of vitamin D) is initiated or completed prior to engaging the intra-oral light therapy apparatus with the patient or prior to removing the intra-oral light therapy apparatus from the patient. In other embodiments, the vitamin D treatment regimen is initiated or completed subsequent to engaging the intra-oral light therapy apparatus with the patient or subsequent to removing the intra-oral light therapy apparatus from the patient. The vitamin D treatment regimen can be in progress during light therapy administration.

Vitamin D can be administered to the patient prior to, currently with, or subsequent to exerting a force on one or more teeth of the patient. The force can be, for example, a heavy force, a force exerted by an orthodontic appliance, or a force exerted by a functional appliance. In one or more embodiments, the force can be less than a heavy force. In one or more embodiments, the vitamin D is administered to the patient prior to initiation of exerting a force on one or more teeth of the patient, or prior to the completion of exerting a force on one or more teeth of the patient. In one or more embodiments, a dosage of vitamin D is administered at a period of time (e.g., seconds, minutes, hours, days, weeks, months) prior to initiation of exerting a force on one or more teeth of the patient or prior to completion of exerting a force on one or more teeth of the patient. In other embodiments, a dosage of vitamin D is administered at a period of time (e.g., seconds, minutes, hours, days, weeks, months) subsequent to initiation of exerting a force on one or more teeth of the patient or subsequent to completion of exerting a force on one or more teeth of the patient. In one or more embodiments, a vitamin D treatment regimen (which can span one or more doses of vitamin D) is initiated or completed prior to initiation of exerting a force on one or more teeth of the patient or prior to completion of exerting a force on one or more teeth of the patient. In other embodiments, the vitamin D treatment regimen is initiated or completed subsequent to the initiation of exerting a force on one or more teeth of the patient or subsequent to completion of exerting a force on one or more teeth of the patient. The vitamin D treatment regimen can be in progress while exerting a force on one or more teeth of the patient.

Vitamin D can be administered to the patient prior to, concurrently with, or subsequent to installing one or more orthodontic appliances on the patient's teeth or functional appliances in the patient's oral cavity. In one or more embodiments, the vitamin D is administered to the patient prior to removing one or more orthodontic appliances from the patient's teeth. In one or more embodiments, a dosage of vitamin D is administered at a period of time (e.g., seconds, minutes, hours, days, weeks, months) prior to installing one or more orthodontic appliances on the patient's teeth or prior to removing one or more orthodontic appliances from the patient's teeth. In other embodiments, a dosage of vitamin D is administered at a period of time (e.g., seconds, minutes, hours, days, weeks, months) subsequent to installing one or more orthodontic appliances on the patient's teeth or subsequent to removing one or more orthodontic appliances from the patient's teeth. In one or more embodiments, a vitamin D treatment regimen (which can span one or more doses of vitamin D) is initiated or completed prior to installing one or more orthodontic appliances on the patient's teeth or prior to removing one or more orthodontic appliances from the patient's teeth. In other embodiments, the vitamin D treatment regimen is initiated or completed subsequent to the installing one or more orthodontic appliances on the patient's teeth or subsequent to removing one or more orthodontic appliances from the patient's teeth. The vitamin D treatment regimen can be in progress while an orthodontic appliance is installed on the patient's teeth.

The administration of vitamin D can increase the amount of tooth movement compared to treatment methods where vitamin D is not administered. The administration of vitamin D can also increase the rate of tooth movement compared to treatment methods where vitamin D is not administered. In one or more embodiments, the administration of vitamin D increases the velocity of tooth movement by about 5%, about 10%, about 15%, about 20%, about 25%, about 30%, about 40%, about 50%, about 60%, about 70%, about 80%, about 90%, or by any percentage from about 1% to about 90%, relative to treatment methods for regulating tooth movement that do not comprise administering vitamin D. In one or more embodiments, the administration of vitamin D increases the rate of bone remodeling compared to treatment methods where vitamin D is not administered. In one or more embodiments, the administration of vitamin D increases the velocity of bone remodeling by about 5%, about 10%, about 15%, about 20%, about 25%, about 30%, about 40%, about 50%, about 60%, about 70%, about 80%, about 90%, or by any percentage from about 1% to about 90%, relative to treatment methods for regulating bone remodeling that do not comprise administering vitamin D.

The administration of vitamin D can reduce the amount of time that the patient undergoes orthodontic treatment. The administration of vitamin D can also reduce the amount of time that a force is exerted on one or more teeth of the patient. In one or more embodiments, the administration of vitamin D reduces the amount of time that a patient undergoes orthodontic treatment or that a force is exerted on one or more teeth of the patient by about 5%, about 10%, about 15%, about 20%, about 25%, about 30%, about 40%, about 50%, about 60%, about 70%, about 80%, about 90%, or by any percentage from about 1% to about 90%, relative to treatment methods that do not comprise administering vitamin D.

The administration of vitamin D can increase the rate of bone remodeling compared to treatment methods where vitamin D is not administered. The administration of vitamin D can also increase the rate of one or both of bone deposition and resorption compared to treatment methods where vitamin D is not administered. In one or more embodiments, the administration of vitamin D increases the rate of one or both of bone deposition or resorption by about 5%, about 10%, about 15%, about 20%, about 25%, about 30%, about 40%, about 50%, about 60%, about 70%, about 80%, about 90%, or by any percentage from about 1% to about 90%, relative to treatment methods that do not comprise administering vitamin D.

In one example, an adult patient's Vitamin D3 blood-serum level is measured during his routine orthodontic-examination and -records appointment. Laboratory results can indicate that the patient's vitamin D3 serum levels are at 20 ng/ml, which is considered to be deficient and abnormal. In this example, the patient's orthodontic diagnosis is Class I mild crowding with 4 mm of crowding on the upper arch and 4 mm on the lower arch. An orthodontic treatment plan can be formulated to include the installation of a fixed orthodontic appliance with some mild expansion of the upper and lower arches.

In this example, the patient self-administers oral oil-based vitamin D3 capsules at an amount of 6000 IU per day for 3 months to increase and normalize his vitamin D3 serum levels. Laboratory serum testing can optionally be performed again after 3 months of vitamin D3 supplementation. The patient can maintains or adjusts his oral dose of vitamin D3 based on his subsequent lab results. It should be understood that there are a number of alternative oral and/or systemic dosing protocols for vitamin D administration that could be followed to achieve similar results. The dosing protocol outlined above is merely one of many approaches.

In this example, orthodontic treatment can start either after the 3 month period or no more than about three months prior. The orthodontic treatment can comprise placing on the patient's teeth conventional fixed orthodontic brackets and/or bands. Light can be administered to the patient on a daily basis, for example, for 20 minutes at an intensity of 50 mW/cm² at wavelength of about 850 nm using an intra-oral light therapy apparatus, such as the one shown in FIG. 1. The orthodontic treatment can continue with the finishing of teeth once the arches have been expanded. In this example, it is believed that the active orthodontic treatment would be completed in 50% to 75% less time than orthodontic treatment without light therapy due to the combination of daily administration of light and vitamin D3 supplementation.

At a passive stage of orthodontic treatment, i.e., retention phase, a fixed retention orthodontic appliance can be installed on the patient's teeth. In one or more embodiments, a Hawley retainer, which is a removable appliance that is designed to maintain tooth position of the anterior teeth, is installed on a patient's anterior teeth. In one or more embodiments, a fixed retainer appliance, such as one including orthodontic brackets, is bonded to the upper 6, lower 6, or upper 6 and lower 6, anterior teeth. The patient can continue with vitamin D3 supplementation. In some examples, the patient self-administers 2000 IU per day to 12,000 IU orally per day. The dosage can be determined based on vitamin D blood serum levels which can be measured periodically to determine dosing. As a result, alveolar bone density around the teeth can be increased during the passive stage. During the passive stage, the patient can be administered once per week with light having a wavelength of about 625 nm using an intra-oral light therapy apparatus, such as the light therapy apparatus shown in FIG. 23 and/or FIG. 28, in areas of the upper and lower arch.

Administering Light Treatment

Light can be administered to the patient using an intra-oral apparatus (including, but not limited to, any intra-oral apparatus or light therapy apparatus, such as apparatus 2500, 3500, described herein) in any of the following ways.

Light can be administered to a region of the patient's mouth. Some examples of these regions include, but are not limited to, one or more teeth (e.g., incisor, canine, premolar, or molar, such as a maxillary central incisor, maxillary lateral incisor, maxillary canine, maxillary first premolar, maxillary second premolar, maxillary first molar, maxillary second molar, maxillary third molar, mandibular central incisor, mandibular lateral incisor, mandibular canine, mandibular first premolar, mandibular second premolar, mandibular first molar, mandibular second molar, or mandibular third molar), a root of one or more teeth (e.g., wherein a root of a tooth can include a portion of one or more roots supporting the tooth, one root supporting the tooth, a plurality of roots supporting the tooth, or all of the roots supporting the tooth), tissue supporting one or more teeth, a portion of the maxilla (e.g., portion of the patient's maxillary alveolar bone), a portion of the mandible (e.g., portion of the patient's mandibular alveolar bone), alveolar mucosa, basal tissue, gingiva (e.g., alveolar soft tissue), periodontal ligaments, cementum, periodontium, a region of a jaw bone or tissue, or at least a portion of the patient's other oral soft tissue or bone tissue. The region can be located on a left side or right side of the patient's mouth. In one or more embodiments, one or more regions are located on both the left and right side of the patient's mouth. In one or more embodiments, the region can be located in the front of the patient's mouth. The region can comprise one, two, three, four, five, six, seven, eight, or more teeth, or tissue surrounding or supporting the teeth. The region can comprise one or more roots of one, two, three, four, five, six, seven, eight, or more teeth, or periodontium of teeth. Regions can comprise tissue (e.g., alveolar or basal tissue) surrounding or supporting any of the teeth specifically described with or without including the tooth itself. Regions can comprise teeth or tissue supported by the maxilla or teeth supported by the mandible. One or more regions can be adjacent to one another, continuous with one another, or separate from one another. Any description herein of regions or examples of regions can apply to any other region or examples of treatment regions provided herein.

In one or more embodiments, light irradiates a region that can comprise a portion of tissue (e.g., bone tissue, or soft tissue) or other regions within the patient's oral cavity without irradiating one or more other portions of the patient's oral cavity. For example, light can irradiate the mandibular first molar on the right side of the patient's oral cavity without irradiating the mandibular third molar that is also located on the right side of the patient's oral cavity. In one or more embodiments, light is administered to one or more roots of only one tooth root and to only one periodontium. Alternatively, light is administered to one or more roots of a plurality of teeth and to a plurality of periodontia. Light can be administered to one or more roots of all or less than all the teeth and periodontia in the patient's oral cavity. One or more selected teeth, roots or periodontia can be irradiated with light. For example, the mandibular first molar and the mandibular third molar on the right side of the patient's oral cavity can be irradiated without the mandibular second molar being irradiated.

In one or more embodiments, light is administered to a patient's alveolar soft tissue, wherein an effective amount of light is irradiated from one or more emitters of an apparatus of the invention. In one or more embodiments, the alveolar soft tissue is alveolar mucosa.

In one or more embodiments, light from an intra-oral apparatus can irradiate a region that comprises a portion of tissue (e.g., bone tissue, or soft tissue) at a much greater intensity than it irradiates other portions of the patient's tissue within the mouth. For example, light can irradiate a first tissue region (e.g., the region of tissue covered by panel 2 shown in FIG. 23) at an intensity that is 3×, 5×, 10×, 20×, 50×, or 100× greater than the intensity that irradiates any other region or portion of the patient's tissue (e.g., the regions of tissue covered by remaining panels 1 and 3-6 shown in FIG. 23). In one or more embodiments, light can irradiate a portion of a patient's alveolar soft tissue at a greater intensity than that of light that irradiates any of the patient's teeth. In another embodiment, light can irradiate or be focused with a greater intensity on the one or more teeth upon which heavy forces are optionally applied (that are desired to be moved), relative to the one or more teeth on which heavy forces are not exerted. Teeth with lower forces or anchorage teeth can be selectively shielded from light or irradiated at lower light intensity so that they can move less and the anchorage effect can be enhanced. In one or more embodiments, this is achieved by applying to the intra-oral apparatus, or adjusting within the intra-oral apparatus, one or more masks that shield from light one or more non-regions, as described with respect to FIG. 27. In one or more embodiments, light reaching a region can have an intensity that is greater than a threshold value. In one or more embodiments, this is achieved by applying to the intra-oral apparatus, or adjusting within the intra-oral apparatus, the density of emitters adjacent one or more regions, as described with respect to FIG. 41. In one or more embodiments, the threshold value can have an intensity as described elsewhere herein.

In one or more embodiments, the region can be close to a surface within the patient's mouth, or within a soft tissue or bone tissue. The region can be at a depth from the surface within the patient's mouth. For example, the region can be about 1 μm, about 1 μm, about 10 μm, about 50 μm, about 100 μm, about 200 μm, about 300 μm, about 500 μm, about 750 μm, about 1 mm, about 2 mm, about 3 mm, about 4 mm, about 5 mm, about 7 mm, about 10 mm, about 15 mm, about 20 mm, about 25 mm, about 30 mm, about 40 mm, about 50 mm, about 60 mm, or about 70 mm from the surface within the patient's mouth. Light can irradiate a region, which can have an area greater than, less than, or about 1 nm2, about 1 μm2, about 0.1 mm2, about 0.2 mm2, about 0.3 mm2, about 0.4 mm2, about 0.5 mm2, about 0.7 mm2, about 1 mm2, about 10 mm2, about 0.2 cm², about 0.5 cm², about 1 cm², about 2 cm², about 3 cm², about 5 cm², about 7 cm², about 10 cm², about 15 cm², about 20 cm², about 25 cm², about 30 cm², about 35 cm², about 40 cm², about 50 cm², about 60 cm², about 80 cm², about 100 cm², about 120 cm², about 140 cm², about 160 cm², about 180 cm² or about 200 cm². Light can irradiate one area, a plurality of areas, a point, or a plurality of points. In one or more embodiments, light irradiates a particular area without irradiating with significant intensity surrounding areas. For example, light can irradiate a particular tooth or set of teeth without significant amounts of light irradiating adjacent teeth. In one or more embodiments, irradiating a tooth comprises irradiating an exposed surface of the tooth, a tooth root, or a periodontium of the tooth (see, for example. FIG. 26 and associated description).

The light administered by an intra-oral apparatus can be emitted from multiple light sources (e.g., emitters 32 shown in FIG. 25A). Light can irradiate a continuous region or one or more discrete regions based on the location of the light sources or emitters within the intra-oral apparatus, as described herein. Light can irradiate various regions from different directions. For example, light can be administered from a right side of a patient's mouth (e.g., from panel 1 shown in FIG. 23) and from a left side of a patient's mouth (e.g., from panel 3 shown in FIG. 23). The light sources or emitters can be adjusted within an intra-oral apparatus such that the administered light is angled upward toward a region, or angled downward to toward a region. The light source or emitters can be displaced, can be angled, can be rotated, or any combination thereof within the intra-oral apparatus.

As described herein, an effective amount of light can be administered via the intra-oral apparatus. An effective amount of light is an amount of light that is effective for regulating tooth-movement; reducing, preventing or minimizing tooth-root resorption; reducing bone resorption, inflammatory dentin resorption or cementum resorption; preventing or minimizing inflammation, or remodeling of tissue surrounding one or more teeth upon which heavy forces are or were exerted; regenerating maxillary or mandibular alveolar bone; or for other methods disclosed herein. The light's properties can include, but are not limited to: its intensity, wavelength, coherency, range, peak wavelength of emission, energy density, continuity, pulsing, duty cycle, frequency or duration.

In one or more embodiments, a method for regulating tooth movement can further comprise determining an effective amount of light. The determination can be based on an intended tooth movement regulation effect. The method can further comprise selecting one or more light properties to provide the effective amount of light. The method can further comprise receiving instructions from a controller, and emitting light having particular properties. The controller can be, for example, controller 430 shown in FIG. 29, the external electronic device described with respect to FIG. 39, or any other controller described herein. The controller can implement any of the steps described herein.

Light can be administered from one or more light source within an intra-oral apparatus capable of irradiating light having intended properties. As described herein, the intra-oral apparatus can emit light from one or more light emitters, such as emitters 32, 132, 232, and/or 332. In one or more embodiments, the intra-oral apparatus comprises about 10 to about 15 emitters, about 15 to about 20 emitters, about 20 to about 30 emitters, about 30 to about 40 emitters, about 40 to about 50 emitters, about 50 to about 70 emitters, or about 70 emitters to about 100 emitters. For example, light can be administered from one or more of the following emitters: a light-emitting diode (LED), which can be present in an array; and a laser, for example, a vertical cavity surface emitting laser (VCSEL) or other suitable light emitter such as an Indium-Gallium-Aluminum-Phosphide (InGaAlP) laser, a Gallium-Arsenic Phosphide/Gallium Phosphide (GaAsP/GaP) laser, or a Gallium-Aluminum-Arsenide/Gallium-Aluminum-Arsenide (GaAlAs/GaAs) laser. In one or more embodiments, the intra-oral apparatus comprises a plurality of lasers. A plurality of light emitters can emit light at one or more different wavelengths. Alternatively, one or more light emitters can emit light at the same wavelength. The one or more light emitters can be arranged on or within the intra-oral apparatus in any manner, such as a linear array or another arrangement described herein.

An effective amount of light can have an intensity that is effective for regulating tooth movement. In one or more embodiments, the light intensity is at least about 10 mW/cm². In other embodiments, the light intensity is about 1 mW/cm² or greater, about 3 mW/cm² or greater, about 5 mW/cm² or greater, about 7 mW/cm² or greater, about 12 mW/cm² or greater, about 15 mW/cm² or greater, about 20 mW/cm² or greater, about 30 mW/cm² or greater, about 50 mW/cm² or greater, about 75 mW/cm² or greater, about 100 mW/cm² or greater, about 200 mW/cm² or greater, about 500 mW/cm² or greater, or about 1 W/cm² or greater. In other embodiments, the light intensity is about 20 mW/cm² or less, about 30 mW/cm² or less, about 50 mW/cm² or less, about 75 mW/cm² or less, about 100 mW/cm² or less, about 200 mW/cm² or less, about 500 mW/cm² or less, about 1 W/cm² or less, about 2 W/cm² or less, about 5 W/cm² or less, or about 10 W/cm² or less. In one or more embodiments the light intensity ranges from about 1 mW/cm² to about 10 W/cm². In another embodiment, the light intensity's lower range is about 3 mW/cm², about 5 mW/cm², about 7 mW/cm², about 12 mW/cm², about 15 mW/cm², about 20 mW/cm², about 30 mW/cm², about 50 mW/cm², about 75 mW/cm², about 100 mW/cm², about 200 mW/cm², about 500 mW/cm², or about 1 W/cm². In another embodiment, the light intensity's upper range is about 20 mW/cm², about 30 mW/cm², about 50 mW/cm², about 75 mW/cm², about 100 mW/cm², about 200 mW/cm², about 500 mW/cm², about 1 W/cm², about 2 W/cm², about 5 W/cm², or about 10 W/cm². In yet another embodiment, the light intensity is at 15 mW/cm². Light can be administered having an intensity having a range determined by any of the intensities described herein. In one or more embodiments, the intensity is an average intensity. In one or more embodiments, the light has an intensity in the range of about 10 mW/cm² to about 60 mW/cm², or about 20 mW/cm² to about 60 mW/cm². In such embodiments, the peak light intensity can about 50 mW/cm² or greater. A peak wavelength is the wavelength at which the highest intensity of light is emitted. In one or more embodiments, light can be pulsed. In other embodiments, the output of light is continuous. In one or more embodiments, the light intensity can vary over time in a cyclical or non-cyclical fashion. The light intensity can vary with or without pulsing. In one or more embodiments, pulse width modulation can be useful for affecting a desired light intensity. If one or more wavelengths of light are administered, then each wavelength can be administered at its own intensity. In one or more embodiments, administering an effective amount or dosage of light can comprise administering light having an intensity of about 15 mW/cm² for less than or up to three minutes duration. Additional details regarding effective amounts or dosages of light are described herein.

In one or more embodiments, an effective amount of light can include light having a wavelength that is of a particular range, or light of a range of wavelengths. The light is not necessarily visible light. For example, the light can include infrared light or near-infrared light. The light can also be provided in the visible light region. Light can be administered having one or more wavelengths ranging from about 620 nm to about 1000 nm. In one or more embodiments, administered light has one or more wavelengths ranging from about 585 nm to about 665 nm, about 815 nm to about 895 nm, about 640 nm to about 680 nm, or about 740 nm to about 780 nm, or any particular wavelength or range of wavelengths, such as, for example, about 625 nm or about 855 nm, or about 605 nm to about 645 nm, or about 835 nm to about 875 nm. In one or more embodiments, the administered light has one or more wavelengths from about 605 nm to about 645 nm, or from about 835 nm to about 875 nm. In one or more embodiments, the administered light has one or more wavelengths from about 615 nm to about 635 nm, or from about 845 nm to about 865 nm. In one or more embodiments, the wavelengths of the administered light are about 625 nm or about 855 nm. In additional embodiments, the administered light has one or more wavelengths ranging from about 400 nm to about 1200 nm. In particular embodiments, the administered light has one or more wavelengths ranging from about 500 nm to about 700 nm, about 585 nm to about 665 nm, about 605 nm to about 630 nm, about 620 nm to about 680 nm, about 815 nm to about 895 nm, about 820 nm to about 890 nm, about 640 nm to about 680 nm, or about 740 nm to about 780 nm. In one or more embodiments the administered light has one or more wavelengths in one or both of the following wavelength ranges: about 820 to about 890 nm and about 620 to about 680 nm. In one or more embodiments, the administered light has one or more wavelengths in the ranges of about 820 to about 890 nm and about 620 nm to about 680 nm. In one or more embodiments, the administered light has one or more wavelengths in the ranges of about 815 to about 895 nm and about 585 to about 665 nm. The administered light can alternatively have one or more wavelengths in one or more of the following ranges: about 613 nm to about 624 nm, about 667 nm to about 684 nm, about 750 nm to about 773 nm, about 812 nm to about 846 nm. In one or more embodiments, the light wavelength's lower range is about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm. In another embodiment, the light wavelength's upper range is about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 935 nm, about 975 nm, about 1000 nm, about 1050 nm, about 1100 nm, or about 1200 nm.

The wavelengths of light administered comprise or consist of the wavelength values described herein.

For example, in one or more embodiments, light administered to a region does not comprise one or more wavelengths exceeding one or more of the following: about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm. For example, in one or more embodiments, no light exceeding about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm is administered to a selected region. In one or more embodiments, light administered to a region does not comprise one or more wavelengths below one or more of the following: about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm. For example, in one or more embodiments, no light below about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm is administered to a selected region. In one or more embodiments, the light administered does not comprise a wavelength of about 600 nm or less. In one or more embodiments, the light administered does not comprise a wavelength of about 1000 nm or greater. In one or more embodiments, the light administered does not comprise a wavelength of about 600 nm or less and does not comprise a wavelength of about 1000 nm or greater.

In one or more embodiments, light administered to a region with a sufficient intensity to be an effective amount in the present methods does not comprise one or more wavelengths exceeding one or more of the following: about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm. For example, in one or more embodiments, no light having a sufficient intensity to be an effective amount for oral or maxillofacial bone remodeling and exceeding about 585 nm, about 605 nm, about 624 nm, about 630 nm, about 635 nm, about 645 nm, about 655 nm, about 660 nm, about 665 nm, about 680 nm, about 684 nm, about 700 nm, about 725 nm, about 755 nm, about 773 nm, about 780 nm, about 795 nm, about 815 nm, about 830 nm, about 846 nm, about 855 nm, about 865 nm, about 875 nm, about 890 nm, about 895 nm, about 905 nm, about 910 nm, about 915 nm, about 920 nm, about 935 nm, about 975 nm, about 1000 nm, about 1050 nm, about 1100 nm, or about 1200 nm is administered to a selected region. In one or more embodiments, light administered to a region with a sufficient intensity to be an effective amount in the present methods does not comprise one or more wavelengths exceeding one or more of the following: about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm. For example, in one or more embodiments, no light having a sufficient intensity to be an effective amount in the present methods and below about 400 nm, about 450 nm, about 500 nm, about 550 nm, about 585 nm, about 595 nm, about 605 nm, about 613 nm, about 615 nm, about 620 nm, about 624 nm, about 625 nm, about 640 nm, about 650 nm, about 667 nm, about 680 nm, about 710 nm, about 740 nm, about 750 nm, about 770 nm, about 812 nm, about 815 nm, about 820 nm, about 835 nm, about 845 nm, or about 860 nm is administered to a selected region. In one or more embodiments, the light administered does not comprise a wavelength of about 600 nm or less having a sufficient intensity to be an effective amount for the present methods. In one or more embodiments, the light administered does not comprise a wavelength of about 1000 nm or greater having a sufficient intensity to be an effective amount for the present methods. In one or more embodiments, the light administered does not comprise a wavelength of about 600 nm or less having a sufficient intensity to be an effective amount for the present methods and does not comprise a wavelength of about 1000 nm or greater having a sufficient intensity to be an effective amount for the present methods.

In one or more embodiments, particular treatments respond better to specific wavelength ranges. For example, in one or more embodiments, tooth movement (or, more particularly, rapid tooth movement) is more effective when the amount of light administered has a wavelength from about 700 nm to about 900 nm. For example, the effective amount of light can have a wavelength of about 850 nm. In one or more embodiments, the intra-oral apparatus irradiates light having a wavelength of about 850 nm and with an intensity of less than 100 mW/cm² continuous wave. In one or more embodiments, bone healing or bone grafting is more effective when the amount of light administered has a wavelength from about 600 nm to about 700 nm.

In one or more embodiments, light is administered at a wavelength sufficient to produce a bactericidal and/or bacteriostatic effect on the patient's teeth and/or oral mucosa. In other words, light can be administered at a wavelength sufficient to kill and/or prevent reproduction of bacteria on the patient's teeth and/or oral mucosa. For example, the light can be administered at a blue or similar wavelength, such as a wavelength of from about 450 nm to about 495 nm. In one or more embodiments, the bactericidal- and/or bacteriostatic-effective light can be administered concurrently with administration of light effective to accelerate tooth movement. For example, in one or more embodiments, light can concurrently be administered to the patient at a blue wavelength, for the resulting bactericidal and/or bacteriostatic effect, and at a red to infrared wavelength to accelerate tooth movement (e.g., towards alignment). In one or more embodiments, the bactericidal- and/or bacteriostatic-effective light can be administered prior to administration of light effective to accelerate tooth movement. In one or more embodiments, the bactericidal- and/or bacteriostatic-effective light can be administered subsequent to administration of light effective to accelerate tooth movement.

In one or more embodiments, light is administered at one, two, or more of the light ranges described. In some instances, light is not administered outside of one, two, or more of the light ranges described. In other embodiments, administered light has other wavelengths, as desired for a particular application. In one or more embodiments, light having a first set of characteristics (e.g., wavelength, intensity, pulsing, timing) can be administered to a first region (e.g., the region at the panel 1 shown in FIG. 23), and light with a second set of characteristics can be administered to a second region (e.g., the region at panel 3 shown in FIG. 23). The first region and the second region can be the same region, can partially overlap, or can not overlap. The first set of characteristics can be the same as the second set of characteristics, can partially overlap with the second set, or can all be different from the second set. In one or more embodiments, one region of a jaw can receive light having a wavelength of a first wavelength range, while another region of the jaw can receive light having a wavelength of a second wavelength range. The first and second wavelengths can overlap. Alternatively, in other embodiments, the first and second wavelengths do not overlap.

In one or more embodiments, one or more wavelengths of light can be sequentially or simultaneously administered to the patient. For example, an intra-oral apparatus of the invention can comprise a first emitter that emits light having a wavelength of about 850 nm and a second emitter that sequentially or simultaneously emits light having a wavelength of about 620 nm. In one or more embodiments, the first emitter can be configured to emit light during a first period of time and the second emitter can be configured to emit light during a second period of time following the first period of time. Stated another way, the second emitter emits light having a wavelength of about 850 nm after the first emitter begins emitting light having a wavelength of about 620 nm, or the second emitter emits light having a wavelength of about 620 nm after the first emitter begins emitting light having a wavelength of about 850 nm. In one or more embodiments, light having a wavelength of about 850 nm is administered daily to a patient until one or more of the following orthodontic treatment phases are complete or almost complete: the alignment phase, the space-closure phase, the finishing-or-detailing phase or the retention phase. Once one or more of these phases are complete or almost complete, the patient can begin receiving a blended light treatment, which comprises administering light having a wavelength of, e.g., about 850 nm and about 620 nm. The about 850 nm wavelength of light can be administered to the patient sequentially or simultaneously with the about 620 nm wavelength of light. Once the teeth have moved into their final position, the passive stage of orthodontic treatment can begin and the patient can begin receiving light having a wavelength of about 620 nm only.

Although examples of light wavelength ranges are provided below for different applications, light having any other light wavelength value, which can include those described herein, can be administered for those applications.

In one or more embodiments, administering light having a wavelength in the range of about 815 nm to about 895 nm, such as about 835 nm to about 875 nm, or about 855 nm, is useful in the present methods, in one or more embodiments, for increasing the rate of movement of teeth. In one or more embodiments, increasing the rate of tooth movement does not increase the tipping motion of teeth beyond that which is experienced by orthodontic patients who are not provided with light via the intra-oral apparatus. In one or more embodiments, administering light having a wavelength in the range of about 585 nm to about 665 nm, such as about 605 nm to about 645 nm, or about 625 nm, is likewise useful in the present methods, in one or more embodiments, for increasing the rate of movement of teeth. In one or more embodiments, administering light having any of the aforementioned wavelengths, in combination with using a functional appliance, exerting a heavy force and/or administering vitamin D, is useful in the present methods, in one or more embodiments, for increasing the rate of movement of teeth.

In one or more embodiments, administration of light having a wavelength in the range of about 585 nm to about 665 nm increases the amount or extent of bodily tooth movement to a greater degree than administration with light having a wavelength in the range of about 815 nm to about 895 nm. In such embodiments, administering light having a wavelength in the aforementioned ranges, in combination with a using functional appliance, exerting a heavy force and/or administering vitamin D can further increase the amount or extent of bodily tooth movement to a greater degree than administering light alone. Administering light having a wavelength in the range of about 585 nm to about 665 nm (e.g., about 625 nm) can result in about 10% to about 50% less tipped movement than the administration of light having a wavelength in the range of about 815 nm to about 895 nm (e.g., about 855 nm). For example, about 10%, about 15%, about 20%, about 25%, about 30%, about 35%, about 40%, about 45%, or about 50% less tipped movement can occur. Particular wavelengths of light can minimize tipped movement. In one or more embodiments, particular wavelengths administered in combination with using a functional appliance, exerting a heavy force and/or administering vitamin D can further minimize tipped movement of teeth.

Thus, in one or more embodiments, administration of light having a wavelength in the range of about 605 nm to about 645 nm, such as about 625 nm, is useful in the present methods, in one or more embodiments, for facilitating the bodily movement of teeth in orthodontic treatment and optionally increasing bone regeneration. In one or more embodiments, the methods further comprise increasing bone regeneration. In another embodiment, administration of light having a wavelength in the range of about 835 to about 875 nm, such as about 855 nm, is useful in the present methods, in one or more embodiments, for increasing the rate of movement of teeth for which some degree of tipped movement is desirable or acceptable and optionally increasing bone regeneration. In the aforementioned embodiments, administering light of these respective ranges, in combination with using a functional appliance, exerting a heavy force and/or administering vitamin D, can be useful, for example, for facilitating the bodily movement of teeth in orthodontic treatment and optionally increasing bone regeneration.

In other embodiments, administration of light having a wavelength as described herein, in one or more embodiments in the range of about 605 nm to about 645 nm, such as about 625 nm, is useful for increasing the quality or degree of bone remodeling. Accordingly the present invention further relates to methods for increasing the quality or degree of bone remodeling, comprising administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods for increasing the quality or degree of bone remodeling further comprise allowing a heavy force to be exerted on one or more teeth of a patient in need thereof and/or administering vitamin D to a patient.

Bone remodeling can include changes in any bone characteristic, such as, but not limited to, bone shape, bone volume, bone density, or bone mineral content. In one or more embodiments, bone remodeling can include bone growth or resorption. Effecting bone growth or bone resorption can result in altering bone shape or position (i.e., tooth movement). Increasing the quality or degree of bone remodeling can aid in adjusting the shape or position of bone (such as a mandibular bone or maxillary bone), or can aid in increasing the retention of teeth in a particular position, for example, in a position resulting from orthodontic treatment, such as an appliance of one or more orthodontic appliances, decreasing the potential for teeth to move back to a previous position, for example, a position prior to orthodontic treatment, such as any appliance of one or more orthodontic appliances. Thus, administration of light having a wavelength in the range of about 585 nm to about 665 nm, or about 605 nm to about 645 nm, or about 615 am to about 635 nm, or about 625 nm, optionally also with light in the range of 815 nm to 895 nm, can be useful in the present methods, for example, for stabilizing the movement of teeth prior to, subsequent to or concurrently with orthodontic treatment. Accordingly, in other embodiments, the present methods further comprise performing orthodontic treatment, such as installing one or more orthodontic appliances on the patient, prior to, subsequent to or concurrently with the administration of light via the intra-oral apparatus. The intra-oral apparatus can be configured to be worn over the orthodontic appliance, or at least a portion of the orthodontic appliance, during the administration of light. In one or more embodiments the orthodontic appliance is a retainer device or a passive orthodontic appliance. Other suitable orthodontic appliances can include, for example, removable retainers such as a Hawley retainer or a vacuum formed retainer, or fixed retainers such as a bonded lingual retainer. These appliances can assist in maintaining tooth position prior to, subsequent to or concurrently with the administration of light, for example, by stimulating bone regeneration or remodeling. In one or more embodiments, the present methods further comprise regulating oral or maxillofacial bone remodeling, such as installing one or more functional appliances to a patient prior to, subsequent to or concurrently with the administration of light. Administration with light having a wavelength in the range of about 815 nm to about 895 nm, or about 835 nm to about 875 nm, or about 845 nm to about 865 nm, or about 855 nm, can also be useful for stabilizing tooth movement, in one or more embodiments prior to, subsequent to or concurrently with orthodontic treatment. In one or more embodiments administration of light having wavelengths in the range of about 585 nm to about 665 nm increases bone regeneration or remodeling to a greater degree or extent that does administration of light having wavelengths in the range of about 815 nm to about 895 nm.

Tooth-root resorption can include breakdown or destruction, or subsequent loss, of the root structure of a tooth. Tooth-root resorption can be caused by differentiation of macrophages into osteoclasts in surrounding tissue which, if in close proximity to the root surface can resorb the root surface cementum and underlying root dentine. Tooth-root resorption can be exacerbated by heavy or supra-physiologic orthodontic forces that exert on periodontal tissue pressure that is higher than the normal physiologic capillary and interstitial pressure. This prevents normal blood flow, which can cause schema (lack of blood supply) and ultimately cell death of soft tissue and bone in the periodontium. These dead tissues, also known as a “hyalinized zone,” are removed through multi-nucleated cells and undermining respiration process and in many cases healthy bone, cementum and dentin are resorbed through this process.

Accordingly, administering light having a particular wavelength, is useful for modulating the speed, quality and type of tooth movement, e.g., bodily versus tipped, and for increasing or stabilizing tooth movement. In one or more embodiments, stabilizing tooth movement can comprise moving one or more teeth with less tipped movement. Stabilizing tooth movement can also comprise retarding or arresting tooth movements in particular ways. For example, this can include minimizing the amount of, or eliminating, slanting (or tipped movement). Administration of light can also be useful for increasing (or inducing) bone regeneration or remodeling. Administration of light can also be useful for reducing, minimizing, or preventing tooth root resorption, bone resorption, inflammatory dentin or cementum resorption, or inflammation of tissue. Administering light, in combination with using a functional appliance, exerting a heavy force, and/or administering vitamin D, can further be useful for these purposes.

In one or more embodiments, the light can be administered to at least a portion of a patient's alveolar soft tissue or other oral tissue, or to it entirely. Alternatively, using an intra-oral apparatus, light of one or more particular wavelengths can be administered to different selected regions of a patient's alveolar soft tissue in order to effect movement of teeth (e.g. anchor (no movement), bodily, or tipped) in one or more regions of a patient's mouth. For example, one or more regions in which it is desired that the teeth not be moved, or that the teeth serve as an anchor to facilitate movement of teeth in other selected regions of a patient's jaw, can be optionally screened or masked such that they receive no light, as described herein with reference to FIG. 5. Alternatively, the one or more regions in which it is desired that the teeth not be moved, or that the teeth serve as an anchor to facilitate movement of teeth in other selected regions of a patient's jaw, do not receive light as the light emitters over such regions are turned off Regions in which it is desired that the teeth be moved bodily can be administered with light having a wavelength in the range of about 585 nm to about 665 nm, in the range of about 605 nm to about 645 nm, about 615 nm to about 635 nm, or about 625 nm. Regions in which it is desired to increase tooth movement but permit some tipped movement of the teeth can be administered with light having a wavelength in the range of about 815 nm to about 895 nm, about 835 nm to about 875 nm, about 845 nm to about 865 nm, or about 855 nm. Tooth movement can be selectively regulated by administering an effective amount of light having one wavelength to one or more selected regions of a patient's alveolar soft tissue, and by administering an effective amount of light having a different wavelength to one or more different selected regions of the mucosa.

In one or more embodiments, light can be administered at a wavelength of a narrow range of wavelengths (e.g., 50 nm or less, 30 nm or less, 20 nm or less, 10 nm or less, 5 nm or less), or at a single wavelength. In one or more embodiments, light is administered at a limited wavelength range (e.g., 1000 nm or less, 700 nm or less, 600 nm or less, 500 nm or less, 400 nm or less, 300 nm or less, 250 nm or less, 200 nm or less, 150 nm or less, 100 nm or less, or 75 nm or less). In one or more embodiments, the light administered does not comprise wavelengths beyond the narrow or limited range of wavelengths. The narrow or limited range of wavelengths can have any of the upper or lower limits of wavelength as described previously. In one or more embodiments, however, the light administered does not comprise light having a sufficient intensity to constitute an effective amount having wavelengths beyond the narrow or limited range of wavelengths.

In one or more embodiments, light can be emitted at one, two, or more peak wavelengths of emission. A peak wavelength is the wavelength at which the highest intensity of light is emitted. In one or more embodiments, light can be administered at a range of wavelengths that comprises a peak wavelength having the highest intensity of the range. In one or more embodiments, a peak wavelength can be at about 620 nm, about 640 nm, about 650 nm, about 655 nm, about 660 nm, about 665 nm, about 670 nm, about 680 nm, about 690 nm, about 800 nm, about 820 nm, about 830 nm, about 835 nm, about 840 nm, about 845 nm, about 850 nm, about 860 nm, about 870 nm, about 890 nm, about 910 or about 930 nm. In one or more embodiments, the administered light does not have wavelengths that vary from the peak wavelength by more than about 1 nm, about 2 nm, about 3 nm, about 5 nm, about 10 nm, about 15 nm, about 20 nm, about 30 nm, about 40 nm, about 50 nm, about 75 nm, about 100 nm, about 150 nm, about 200 nm, about 250 nm, about 300 nm, about 400 nm, or about 500 nm.

Where two or more light wavelengths are administered, the light can be administered at any ratio of each wavelength's intensity. For example, light administered at a first wavelength can have an intensity that is about 1.1×, 1.2×, 1.3×, 1.5×, 1.7×, 2.0×, 2.5×, 3.0×, 3.5×, 4.0×, 5.0×, 10×, 12×, 15×, 20×, 30×, 50×, 100× that of light administered at a second wavelength. In one or more embodiments, the administered light is emitted from one or more light emitters, in another embodiment, from one or more light emitters having a first set of properties and, optionally, from a second set of light emitters having a second set of properties. In other embodiments, the number of light emitters having a first set of characteristics exceeds that of the light emitters having a second set of characteristics. For example, the number of light emitters having the first set of characteristics can be about 1.1×, 1.2×, 1.3×, 1.5×, 1.7×, 2.0×, 2.5×, 3.0×, 3.5×, 4.0×, 5.0×, 10×, 12×, 15×, 20×, 30×, 50×, 100× the number of light emitters having the second set of characteristics, or vice versa.

The light can optionally be monochrome or substantially monochrome, e.g., having a wavelength from about 10 nm less than to about 10 nm greater than a specific wavelength. When light is “substantially monochrome” it has a single wavelength, or comprises light of the single and light of one or more other wavelengths that are emitted at an intensity that is ineffective in the present methods, including for regulating oral or maxillofacial bone remodeling when administered to the oral or maxillofacial bone, muscle, or soft tissue, or one or more teeth of a patient, with or without allowing a functional appliance to exert a force on oral or maxillofacial bone, muscle, or soft tissue, or one or more teeth of the patient. In one or more embodiments, substantially monochrome light is emitted at a narrow range of wavelengths without being emitted at other wavelengths outside the range or without an effective intensity of light being emitted at other wavelengths outside the range. In one or more embodiments, substantially monochrome light comprises light of wavelengths within about 5 nm, about 10 nm, or about 20 nm of a specific wavelength, and either (1) does not comprise light of other wavelengths outside such a range, or (2) does not comprise an effective intensity of light of other wavelengths outside the range. By way of example, substantially monochrome 850 nm light can comprise light of wavelengths ranging from about 830 nm to about 870 nm. Administering light from light emitters that emit at multiple wavelengths can allow for irradiation over multiple wavelengths or greater selectivity and precision in administration. The light can optionally comprise incoherent light. In one or more embodiments, light can be administered at a single frequency, light can have a phase that drifts relatively quickly, a pulse of light waves can have an amplitude that changes quickly, or a light wave can encompass a broad range of frequencies.

Light can be administered directly from a light emitter to a region in the patient's mouth. In one or more embodiments, light can be modified by optics before reaching or traveling through the surface within the patient's mouth (e.g., the alveolar soft tissue). For example, light can be diffused, focused, parallel, reflected, redirected, or filtered after it is emitted and before it reaches or travels through the surface within the patient's mouth. Such modification can be achieved, for example, by using a foil or other suitable material with the intra-oral apparatus, such as the reflective backing 20 depicted in FIG. 3A. In one or more embodiments, light of one or more wavelengths can be selectively blocked or partially filtered before reaching the surface within the patient's mouth. In one or more embodiments, light can diverge or converge from an emission source before reaching the region. For example, light can diverge in a beam having an included angle θ in the range of about 45-60°. The emitted light diverge to have an included angle θ of 0 to about 15°, 0 to about 30°, 0 to about 45°, 0 to about 60°, 0 to about 75°, 0 to about 90°, or 0 to about 120°.

In one or more embodiments, industry standard LEDs are used to produce the light. The LEDs can comprise one or more emitter arrays arranged on a series of treatment arrays to cover the target area of the alveolar mucosa of both the maxilla and mandible.

Light that irradiates the region can optionally have the same or about the same characteristics as light that is emitted. In one or more embodiments, light that reaches the region does not have the same characteristics as the light that is emitted. One or more of the light characteristics can optionally be altered prior to administration or when it passes through the oral tissue of the patient. One or more of the light characteristics can optionally be altered when it passes through optics, such as one or more lenses or mirrors that are coupled to or disposed within the intra-oral apparatus. For example, one or more of the light characteristics can be altered in the range of about ±20% or less, about ±15% or less, about ±10% or less, about ±5% or less, about ±3% or less, about ±1% or less, about ±0.5% or less, or about ±0.1% or less.

The dosage or effective amount of light that irradiates from, for example, the light emitters, can range from about 24 J/cm² to about 200 J/cm². The effective dosage of light can be administered once or repetitively. In one or more embodiments, the effective amount can have an irradiated light energy density that is from about 30 J/cm² to about 100 J/cm². In other embodiments, the effective amount can be about 5 J/cm² or less, about 10 J/cm² or less, about 20 J/cm² or less, about 30 J/cm² or less, about 50 J/cm² or less, about 75 J/cm² or less, about 100 J/cm² or less, about 125 J/cm² or less, about 150 J/cm² or less, about 175 J/cm² or less, or about 200 J/cm² or less. The effective amount of irradiated light can be about 1 J/cm² or more, about 5 J/cm² or more, about 10 J/cm² or more, about 20 J/cm² or more, about 25 J/cm² or more, about 30 J/cm² or more, about 40 J/cm² or more, about 50 J/cm² or more, about 60 J/cm² or more, about 75 J/cm² or more, about 100 J/cm² or less, about 125 J/cm² or more, about 150 J/cm² or more, or about 175 J/cm² or more. The effective amount of irradiated light can be in a range bounded by any of the energy density values described herein. The effective amount of irradiated light can be increased, for example, by using a light source that emits light having a relatively higher average intensity, or by increasing the duration of administration of light.

An effective amount of light can have an energy density that reaches a region, such as the mandibular bone or maxillary bone. For example, an effective amount of light that reaches a region can be from about 0.5 J/cm² to about 100 J/cm². The effective amount of light that reaches the region can be administered once or repetitively. In some other embodiments, the effective amount has an irradiated light energy density that is from about 1 J/cm² to about 50 J/cm². In other embodiments, the effective amount of light is about 0.5 J/cm² or less, about 1 J/cm² or less, about 2 J/cm² or less, about 5 J/cm² or less, about 10 J/cm² or less, about 15 J/cm² or less, about 20 J/cm² or less, about 30 J/cm² or less, about 40 J/cm² or less, about 50 J/cm² or less, about 70 J/cm² or less, about 80 J/cm² or less, about 90 J/cm² or less, or about 100 J/cm² or less. The effective amount of light can be about 0.5 J/cm² or more, about 1 J/cm² or more, about 2 J/cm² or more, about 3 J/cm² or more, about 5 J/cm² or more, about 10 J/cm² or more, about 15 J/cm² or more, about 20 J/cm² or more, about 30 J/cm² or more, about 40 J/cm² or more, about 50 J/cm² or less, about 60 J/cm² or more, about 70 J/cm² or more, or about 80 J/cm² or more. The effective amount of light that reaches the region can be in a range bounded by any of the energy density values described herein.

The duration over which the effective amount, which is optionally repetitive, is administered via the intra-oral apparatus can range from about 10 to about 40 minutes. In other embodiments, dosage can be administered in a period of time of about 30 seconds or more, about 1 minute or more, about 2 minutes or more, about 3 minutes or more, about 5 minutes or more, about 7 minutes or more, about 10 minutes or more, about 15 minutes or more, about 20 minutes or more, about 25 minutes or more, about 30 minutes or more, about 40 minutes or more, about 50 minutes or more, about 1 hour or more, about 1 hour 15 minutes or more, about 1 hour 30 minutes or more, or about 2 hours or more. The effective amount can be administered in a period of time of about 3 minutes or less, about 5 minutes or less, about 10 minutes or less, about 15 minutes or less, about 20 minutes or less, about 25 minutes or less, about 30 minutes or less, about 35 minutes or less, about 40 minutes or less, about 50 minutes or less, about 1 hour or less, about 1 hour 15 minutes or less, about 1 hour 30 minutes or less, about 2 hours or less, or about 4 hours or less. The effective amount can be administered for a range of time based on any of the time values described herein. In one or more embodiments, one or more light blocking masks or shades can be used with the intra-oral apparatus. An oral mask can block one or more wavelengths of light, or can reduce the intensity of one or more wavelengths of light, from reaching a region covered by the oral mask. This can include an upper arch (e.g., maxillary teeth), or lower arch (e.g., mandibulary teeth). In one or more embodiments, the oral mask contacts oral tissue or one or more teeth of a patient.

Any time period can be provided between dosages of light. For example, the time period between dosages can be on the order of seconds, minutes, hours, days, weeks, months, quarter of a year, or years.

The effective amount, which in one or more embodiments is repetitive, can be administered with any desired frequency, e.g., four times daily, three times daily, twice daily, daily, every second day, weekly, biweekly, monthly, or quarterly. In one or more embodiments, dosage can be administered at regular intervals (e.g., daily), while in other embodiments, the dosage is not administered at regular intervals (e.g., administration can occur 2 times a week at any time during the week). In one or more embodiments, light can be administered in the morning and at night. Light can be administered throughout the time period that a patient is undergoing bone remodeling or tooth movement. In one or more embodiments, a patient undergoes orthodontic treatment in addition to undergoing bone remodeling or tooth movement. Orthodontic treatment can occur prior to, subsequent to, or concurrently with oral or maxillofacial bone remodeling. Light can be administered throughout the time period that a patient is undergoing orthodontic treatment, or following treatment to stabilize tooth movement. For example, light can be administered after a functional appliance or an orthodontic appliance is applied, removed, adjusted, after an appointment, or after an active stage, as described herein. It can be desirable to administer light with greater frequency, e.g. four times daily, three times daily, twice daily, daily or every second day, while a patient is undergoing orthodontic treatment. Where light is being administered, for example, to stabilize tooth movement or reduce tooth-root resorption, treatments of reduced frequency, e.g. weekly, biweekly, monthly, or quarterly, can be used to minimize inconvenience to patients. In one or more embodiments, the effective amount of light maintains the ATP energy levels of tissue cells, e.g., ischemic tissue cells, to prevent cell death, as described herein. In one or more embodiments, light is administered no less than about every second day. In one or more embodiments, a patient receives light treatment at least three or four times a week.

Light can be administered for any length of time. In one or more embodiments, light can be administered on the order of seconds, minutes, hours, days, weeks, months, quarters, or years. For example, light can be administered while an orthodontic appliance or a functional appliance exerts a force. One or more dosages of light can be administered over a period of time during which a patient is undergoing oral or maxillofacial bone remodeling during which an orthodontic appliance or a functional appliance exerts a force. In one or more embodiments, one or more dosages of light can be administered over a period of time during which a force is exerted on one or more teeth, during which a patient is wearing an orthodontic appliance that itself can exert a force, such as a heavy force, or during which a patient is undergoing orthodontic treatment during which a force, such as a heavy force can be applied. In one or more embodiments, while a patient is undergoing orthodontic treatment or is wearing a secondary appliance, the patient is administered with light via the intra-oral apparatus. For example, a portion of the intra-oral apparatus (e.g., a mouthpiece) can be configured to be disposed over the orthodontic appliance, such as an orthodontic appliance that can exert a force effective to move one or more teeth, during the light administration. In other embodiments, the intra-oral apparatus exerts a heavy force on one or more teeth such that no secondary appliance is necessary. Administration of light, which can include regular, irregular, continuous or discontinuous administration of light, can be on the order of days, weeks, months, quarters, or years. In one or more embodiments, light is administered over a plurality of days, weeks, months, quarters, or years. In one or more embodiments, light is administered over a plurality of sessions. In one or more embodiments, one or more hours, days, weeks, months, quarters, or years occur between sessions.

If the light emitters are pulsed, then their duty cycle can be adjusted as desired; e.g., light can be administered with a duty cycle of about 10%, about 20%, about 30%, about 40%, about 50%, about 60%, about 70%, about 80%, or about 90%. The pulsing can occur with any frequency. For example, light can be pulsed every picosecond, nanosecond, microsecond, millisecond, second, multiple seconds, or minutes. Frequencies can include, but are not limited to, about 1 mHz, about 10 mHz, about 50 mHz, about 100 mHz, about 500 mHz, about 1 Hz, about 2 Hz, about 5 Hz, about 10 Hz, about 15 Hz, about 20 Hz, about 25 Hz, about 30 Hz, about 35 Hz, about 40 Hz, about 50 Hz, about 70 Hz, about 100 Hz, about 200 Hz, about 500 Hz, or about 1 kHz. Any of the aforementioned characteristics of light emission (e.g., whether the light is on or off, continuous or pulsed, duty cycle, frequency, intensity, wavelength) can be varied or maintained. Where the light is emitted from a source having a controller, any characteristics of light emission can be varied or maintained in accordance with instructions from its controller.

In one or more embodiments, the emitters of the intra-oral apparatus can be controlled so that the number of lights that are on or off at a given period can be individually controllable. For example, a light source or emitter can be turned on or off relative to other light sources or emitters (such as, e.g., the apparatus depicted in FIG. 40C). Various light sources can be modulated individually (e.g., one or more properties of a particular light source can be varied) or otherwise individually controlled, to expose individual sections of a patient's alveolar soft tissue or other regions to a desired energy density. This can be desirable when it is desirable to administer light to different regions (e.g., via the various panels of the intra-oral apparatus). Thus, the position of light being administered can be varied. In another embodiment, different types of light sources can be turned on or off relative to other light emitters. For example, at some times, light emitted in a first wavelength range can be turned on while light emitted in a second wavelength range can be turned off, vice versa, or both types of light emitters can be turned on or off. Thus, the wavelength of light being administered can be varied. In one or more embodiments, the intensity of light being administered can be varied (e.g., by turning some light sources on or off, or varying the intensity emitted by the light sources). Administering light selectively can enable an increased anchorage effect (by reason of lower tooth mobility) of teeth which are not exposed to any light, which can thereby permit for more precise bone remodeling or movement of teeth to which light is administered.

In one or more embodiments, particularly where infrared light is administered to a patient, the present methods further comprise providing emission of a visible light. In one or more embodiments the visible light is bright enough to aid in the apparatus's positioning if performed by a person other than the patient. The visible light can be, but is not necessarily, of a wavelength or wavelength range that is beneficial for light treatment or regulation of tooth movement. In one or more embodiments, the ratio of the intensities of the visible and infrared components of the light is 1 part or less visible light to 5 parts or more infrared light. In other embodiments, the ratio of the intensities of visible and infrared components is about 1 part or more visible light to 5 parts or more infrared light, 1 part or more visible light to 3 parts infrared light, 1 part or more visible light to 2 parts infrared light, 1 part or more visible light to 1 part infra-red light, 2 parts or more visible light to 1 part infrared light, 3 parts or more visible light to 1 part infrared light, 5 parts or more visible light to 1 part infrared light, 10 parts or more visible light to 1 part infrared light, or no infrared light. In one or more embodiments, light can be emitted at a range that can comprise wavelengths less than an order of magnitude relative to one another. Alternatively, the range can comprise wavelengths emitted at one, two, three or more orders of magnitude relative to one another.

The region and desired light characteristics can vary from patient to patient. A physician, dentist, other health-care provider or patient can determine a light treatment regimen for a patient wearing an intra-oral apparatus.

In some instances, it can be desirable to administer light to less than all regions of the patient's alveolar soft tissue, for example, if it is desired that teeth in other regions do not need to be moved (e.g. it can be desired to regulate the movement of only the upper teeth of a patient, or only the lower teeth, or to use particular teeth as an anchor when regulating the movement of other teeth by administering no light to, e.g., blocking light from, the anchor teeth). Administering light to selected regions of the patient's alveolar soft tissue can comprise causing light to irradiate one or more selected tooth roots through the tissue or mucosa.

In one or more embodiments, light is selectively administered to less than all regions of the patient's alveolar soft tissue before, during, or after the exertion of heavy forces. In one or more embodiments, light is not administered to an anchor tooth. In this embodiment, a secondary appliance, such as a functional appliance, is located between the anchor region or tooth and one or more selected bone region sought to be remodeled. The secondary appliance can exert a force on the selected bone region, for example, on another tooth. In one or more embodiments, the force is a heavy force. In one or more embodiments, an effective amount of light is administered to the selected bone region or other tooth and not to the anchor region or anchored tooth via the intra-oral apparatus. The administration of light can increase the rate of the selected bone remodeling region or velocity (or rate of movement) of the other tooth and reduce, minimize, or prevent root resorption of the other tooth, while not increasing the rate of bone remodeling of the non-selected regions or velocity of the anchor tooth.

It can also be desirable to administer light of different wavelengths to different regions of the patient's alveolar soft tissue, if it is desired to differentially manipulate the movement of a patient's teeth, as described herein. For example, light of a first wavelength can be administered to a first region and light of a second wavelength can be administered to a second region. The first and second wavelengths can comprise any wavelengths described elsewhere herein, such as for example, about 585 nm to about 665 nm, or about 815 nm to about 895 nm.

Light can be administered over an area (also referred to herein as a “light irradiation area”). For example, light can be administered to a region with an area. In one or more embodiments, light characteristics (e.g., light intensity) can remain uniform over the area. In other embodiments, light characteristics can vary over the area. For example, light intensity can be uniform or can vary over an area of a region. The area of light administration can have any shape or size.

Light can be administered to a light irradiation area of any size and shape. For example, a region, such as a specified region of the patient's alveolar soft tissue, can have any size or shape. The light irradiation area can have one or more dimensions (e.g., length, width, diameter) that range from about 1 to about 80 mm or from about 1 to about 70 mm. In one or more embodiments, one or more dimensions (e.g., length, width, diameter) of a light irradiation area can range from about 1 to about 3 mm, about 3 to about 5 mm, about 5 to about 7 mm, about 7 to about 10 mm, about 10 to about 15 mm, about 15 to about 20 mm, about 20 to about 25 mm, about 25 to about 30 mm, about 30 to about 35 mm, about 35 to about 40 mm, about 40 to about 50 mm, about 50 to about 60 mm, or about 60 to about 80 mm.

A light-irradiation area can have any shape, which can include, but is not limited to, a rectangular shape, square shape, triangular shape, hexagonal shape, octagonal shape, trapezoidal shape, circular shape, elliptical shape, crescent shape, cylindrical shape or half-circle. In one or more embodiments, the dimensions of a light emitter can be about the same as dimensions for a light irradiation area. In other embodiments, the dimensions of a light source can be greater than the dimensions of a light irradiation area. Alternatively, the dimensions of a light source can be less than the dimensions of the light irradiation area. The relative areas of a light source and light irradiation area can depend on any angle, which can be a parallel, convergence, or divergence angle, at which light is emitted.

In one or more embodiments, an effective amount of light can be provided in a treatment regimen using the intra-oral apparatus. The treatment regimen can be used in the present methods.

In one or more embodiments, a typical treatment regimen provides a dose of light daily. Each of the daily doses of light can be administered over a period lasting from a few minutes to about an hour when the patient is using the intra-oral apparatus. For example, daily ½ hour doses of light can be effective and are not unduly inconvenient for patients. A single daily dose can be as effective as dividing the same dose into multiple sessions administered at different times during the day. Some treatment regimens can comprise administering light in 5 treatments per week for 12 weeks. Each treatment can last ½ hour and irradiate the patient's oral tissue with light having wavelengths of 660 nm and 840 nm. The 660 nm light can have an intensity of about 20 mW/cm² at the skin's surface. The 840 nm light can have an intensity of about 10 mW/cm² at the skin's surface. These treatment regimens can enhance bone density.

Other treatment regimens can comprise administering light in daily treatments for 21 days. Each treatment lasts from about 20 minutes to about one hour and irradiates the tissues of a patient's mouth with light having a wavelength of 618 nm and an intensity of 20 mW/cm² at the skin's surface. These treatment regimens can accelerate healing of bone grafts.

Another treatment regimen can comprise a twice-daily administration of light for six months. Light can be administered, via the intra-oral apparatus, at a wavelength of about 660 nm or about 840 nm, or at both wavelengths. The intensity of the light can be about 20 mW/cm² at the target surface within the patient's mouth. An orthodontic appliance or a functional appliance can be present in the patient's mouth while the light is administered. Subsequent to the first 6 month period, a second 6 month period can be provided where light is administered once every other day. The same functional appliance or one or more orthodontic appliances can be present in the patient's mouth at this time. The administration of light can optionally become less frequent or be administered at a lower intensity as treatment progresses. In one or more embodiments, the same intra-oral apparatus is useful throughout the treatment regimen. In other embodiments, however, one or more different intra-oral apparatus are useful throughout the treatment regimen.

Another treatment regimen can comprise administering light to a patient having an orthodontic appliance or a functional appliance, and then subsequently adjusting the appliance. In one such embodiment, the patient uses both the intra-oral apparatus and the appliance (e.g., braces). The intra-oral apparatus can, for example, fit over the secondary appliance such that the intra-oral apparatus and orthodontic appliance can be used simultaneously. In one or more embodiments, two or more other orthodontic appliances can be used with the intra-oral apparatus. In other embodiments, an orthodontic appliance and a functional appliance are used with the intra-oral apparatus. The orthodontic appliance can be installed on the patient's teeth prior to, subsequent to, or concurrently with the installation of a functional appliance. In one or more embodiments, the orthodontic appliance worn and adjusted is the intra-oral apparatus, which also administers the light. In one or more embodiments, adjusting an orthodontic appliance can increase or alter the magnitude of a force applied on one or more teeth. Adjusting an orthodontic appliance can alter the direction of a force applied on one or more teeth. Light can be administered to one or more selected teeth for up to an hour prior to adjusting an orthodontic appliance. Adjusting the orthodontic appliance can cause a heavy force to be exerted on the one or more teeth. Adjusting the appliance can change the magnitude or direction, or both, of the force exerted. Adjusting the appliance can comprise tightening, loosening or replacing one or more of the appliance's wires, springs or elastic devices. Different sizes, materials, or shapes of such components can be used. Light can then be administered daily to the one or more selected teeth, until the next adjustment of the appliance. This administration of light can reduce, minimize, or prevent tooth-root resorption, bone resorption, tissue inflammation, periodontium resorption or cementum resorption.

Another treatment regimen can comprise administering vitamin D to a patient; administering light to a region of the alveolar soft tissue, or the mandibular bone, maxillary bone, or one or more teeth: installing a functional appliance or an orthodontic appliance; and subsequently adjusting the orthodontic appliance or functional appliance. In one or more embodiments, adjusting a functional appliance or an orthodontic appliance increases or decreases the magnitude of a force exerted on one or more teeth, mandibular bone, maxillary bone, or temporal bone. Adjusting a functional appliance also can alter the direction of a force exerted. Light can be administered to one or more selected regions for up to an hour prior to adjusting a functional appliance or an orthodontic appliance. Adjusting the functional appliance or orthodontic appliance can cause a force to be exerted on the one or more teeth, mandibular bone, maxillary bone, or temporal bone. Adjusting the functional appliance or orthodontic appliance can change the magnitude or direction, or both, of the force exerted. Adjusting the functional appliance or orthodontic appliance can comprise tightening, loosening or replacing one or more of the appliance's wires, springs or elastic devices. Different sizes, materials, or shapes of such components can be used. Light can then be administered, for example, daily, to the one or more selected regions, until the next adjustment of the functional appliance or orthodontic appliance. This administration of light can regulate oral or maxillofacial bone remodeling. In one or more embodiments, the administration of light regulates tooth movement. For example, the administration of vitamin D and administration of light can increase the rate of bone remodeling or tooth movement. This can decrease the amount of time that a functional appliance or an orthodontic appliance is worn or needs to be worn by a patient.

The present methods can further comprise controlling temperature of the apparatus of the invention, the patient's mouth, the patient's alveolar soft tissue or of any light source that is directed at or that contacts the patient's mouth or region thereof. As described herein, the intra-oral apparatus can comprise a temperature sensor (or other like sensor) that monitors the temperature of the patient's mouth, the patient's alveolar soft tissue and/or light emitters. The method can comprise cooling, heating, or maintaining the temperature at a patient's mouth. A patient's mouth, for example, the patient's alveolar soft tissue, can be contacted with a temperature control mechanism, which can cause the removal or provision of heat. In one or more embodiments, such a temperature control mechanism is coupled to or disposed within the intra-oral apparatus. In one or more embodiments, the temperature of the light source can be controlled. The temperature control mechanism can communicate with the light source. Heat can be removed from or provided to the light source. Any embodiments for temperature regulation described herein can be employed in the method. The method can further comprise measuring a temperature of the patient's mouth, measuring a temperature at a particular surface region within the patient's mouth, e.g., the alveolar soft tissue, or measuring a temperature at one or more of the light emitters. Temperature regulation can optionally occur in response to one or more temperature measurements made.

In one or more embodiments the present methods are performed prior to, subsequent to or concurrently with orthodontic treatment of a patient. In one or more embodiments the administration of light is repetitive.

Oral or maxillofacial bone remodeling can occur at the mandibular bone, maxillary bone, or temporal bone. In one or more embodiments, oral or maxillofacial bone remodeling can occur at a joint, such as the temporomandibular joint. The one or more embodiments, oral or maxillofacial bone remodeling can occur at a condyle or glenoid fossa. The regulation of oral or maxillofacial bone remodeling can result in the repositioning of the mandibular bone or maxillary bone, the lengthening or shortening of the mandibular bone or maxillary bone, or altering the angle, shape, or dimensions of the mandibular bone or maxillary bone.

Oral or maxillofacial bone remodeling can comprise installing a functional appliance in a patient. A functional appliance can be present on one or more teeth of a patient. The methods can comprise installing a functional appliance in a patient, such as installing the appliance on one or more teeth, the patient's gums, the patient's maxillary or mandibular bone, or other oral or maxillofacial features of the patient, adjusting a functional appliance of the patient, or can comprise removing a functional appliance from the patient. A treatment for oral or maxillofacial bone remodeling can include a period of time during which the functional appliance is installed in the patient. In one or more embodiments, treatment for oral or maxillofacial bone remodeling can include a period of time after the functional appliance has been installed in or removed from the patient. In one or more embodiments, treatment for oral or maxillofacial bone remodeling can include a period of time preceding the installation of a functional appliance. In other embodiments treatment for oral or maxillofacial bone remodeling includes a period of time prior to, during, or subsequent to the exertion of a force on oral or maxillofacial bone, muscle, soft tissue, or one or more teeth, such as mandibular bone, maxillary bone, temporal bone, or on one or more oral muscles that can prevent the oral muscles from exerting a force on the one or more teeth, mandibular bone, maxillary bone, temporal bone. Treatment for oral or maxillofacial bone remodeling can include a period of time while a patient is seeing or consulting with an orthodontist or other dental specialist.

Treatment for oral or maxillofacial bone remodeling, including methods for regulating such remodeling, can comprise an active stage and a passive stage. An active stage can include some time during which a functional appliance is installed in and/or on the patient. In one or more embodiments, an active stage includes a time during which a force is exerted on a tooth, mandibular bone, maxillary bone, temporal bone. An active stage can include a period during which the patient is undergoing one or more adjustments to the patient's functional appliance. In one or more embodiments, the active stage comprises the alignment phase of orthodontic treatment. A passive stage can include a period after a functional appliance has been removed from the patient. In one or more embodiments, a passive stage includes a period during which a functional appliance is installed, but is no longer undergoing adjustments. In one or more embodiments, a passive stage includes a period during which there is no further muscular tension on the jaw or teeth when the functional appliance is in position, which typically occurs after a period of treatment and bone remodeling. In one or more embodiments, a passive stage includes a period during which a functional appliance is not providing force to effect bone remodeling. Instead, the passive stage can include a period during which a functional appliance is installed in a patient and that maintains the maxillary bone or mandibular bone in its position. Any stage of oral or maxillofacial bone remodeling can last on the order of days, weeks, months, quarters, or years.

An orthodontic treatment can cause one or more teeth to move or maintain its position relative to a supporting maxilla or mandible, or can comprise regulation of tooth movement. In one or more embodiments, orthodontic treatment can comprise reducing or closing the space or gap between teeth, for example, that resulting from an injury, extraction or the like. In some instances, orthodontic treatment can comprise aligning teeth. Orthodontic treatment can comprise treating malocclusion, which can occur when teeth fit together improperly, for example, as a result of their individual positions or positions of underlying jaw bone as they relate to one another. Malocclusion can be treated using light treatment or tooth movement regulation according to the methods described herein. Accordingly, the present invention further relates to methods for treating or preventing malocclusion, comprising administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods further comprise allowing a heavy force to be exerted on one or more teeth of a patient in need thereof. In one or more embodiments, the light is administered before, during or after the heavy force is exerted.

An orthodontic treatment can comprise installing an orthodontic appliance in a patient. An orthodontic appliance can be present on one or more teeth of a patient. The methods can comprise installing an orthodontic appliance in a patient, such as installing the appliance to one or more teeth of the patient, adjusting an orthodontic appliance of the patient, or can comprise removing an orthodontic appliance from the patient. In one or more embodiments, an orthodontic appliance can be installed or removed prior to, subsequent to, or concurrently with the installation or removal of a functional appliance. Orthodontic treatment can include a period of time during which the orthodontic appliance is applied to the patient. In one or more embodiments, orthodontic treatment can include a period of time after the orthodontic appliance has been applied or removed from the patient. In one or more embodiments, orthodontic treatment can include a period of time preceding the application of an orthodontic appliance. In other embodiments orthodontic treatment includes a period of time prior to, during, or subsequent to the exertion of a heavy force on one or more teeth. Orthodontic treatment can include a period of time while a patient is seeing or consulting with an orthodontist.

In one or more embodiments, orthodontic treatment can comprise an active stage and a passive stage. An active stage can include time during which an orthodontic appliance is installed in the patient. In some instances, an active stage can include time during which a force is applied to one or more teeth to effect tooth movement. In one or more embodiments, the force applied to one or more teeth during an active stage is a heavy force. An active stage can include a period during which the patient is undergoing one or more adjustments to the patient's appliance. In one or more embodiments, the active stage includes one or more of the following phases of orthodontic treatment: the alignment phase, a space-closure phase, and a finishing-or-detailing phase. The alignment phase is described herein. The space-closure phase typically occurs after the alignment phase, if the alignment phase is needed. In general, during the space-closure phase, one or more teeth are moved so that any gaps between the teeth are minimized. The finishing-or-detailing phase typically occurs after the space-closure phase, if the space-closure phase is needed. In general, during the finishing-or-detailing phase, square or rectangular wires are installed on one or more teeth of the patient, in one or more embodiments, as part of an orthodontic appliance that comprises brackets, and useful for torqueing one or more of the teeth so that the teeth are set to a final, corrected position. Bodily movement of one or more teeth typically occurs during one or both of the space-closure phase and the finishing-or-detailing phase. Light treatment can be administered to the patient during any one or more of the phases of the active stage or during the passive stage.

A passive stage, which comprises the retention phase, can include a period after an appliance has been removed from the patient. In some instances, a passive stage can include a period during which an appliance is installed but is no longer undergoing adjustments. In some instances a passive stage can include a period during which an orthodontic appliance no longer exerts a force on the teeth. In one or more embodiments, a passive stage can include a period during which, for example, an orthodontic appliance is not providing force to effect movement of a tooth. Instead, the passive stage can include a period during which an appliance is installed in a patient and that maintains one or more teeth in its position. In one or more embodiments, any stage of orthodontic treatment can last on the order of days, weeks, months, quarters, or years.

In one or more embodiments, orthodontic treatment can result in bone remodeling. In one or more embodiments, orthodontic treatment and bone remodeling or tooth movement occurs concurrently. In one or more embodiments, in addition to light treatment, force can be exerted on one or more tooth, any region of the jaw, or any other region of the mouth or head. Force can be exerted by the intra-oral apparatus and/or an orthodontic appliance. In one or more embodiments, the force is a heavy force. Bone remodeling can involve altering the position or morphology of bone, including the jaw bone. For example, a jaw bone can be moved forward, or can be lengthened. Other examples of bone remodeling, as disclosed herein, can also be applicable. In one or more embodiments, bone remodeling can occur in combination with regulating tooth movement. Accordingly, the present methods are useful for and, in one or more embodiments, result in bone remodeling. Light can be administered to a region, such as any oral bone or tissue, and is useful for bone remodeling. Accordingly, the invention further provides methods for inducing bone remodeling, comprising administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods further comprise allowing a heavy force to be exerted on one or more teeth of a patient in need thereof. In one or more embodiments, the light is administered before, during or after the heavy force is exerted. Light treatment can increase the rate of bone remodeling, and can be provided in combination with forces for bone remodeling. For example, administering an effective amount of light as described in the present methods can reduce the amount of time to achieve the same degree of bone remodeling without light by about 10%, about 20%, about 30%, about 40%, about 50%, about 60%, about 70%, about 80%, or about 90%. Light treatment can promote bone remodeling which can increase the rate of teeth movement. This can allow heavier forces to be used, which could accelerate tooth movement even more than with lighter forces. Such forces can be exerted by one or more orthodontic appliances.

Installing, adjusting, or removing of an orthodontic appliance can occur before or after administering an effective amount of light via the intra-oral apparatus. In one or more embodiments, the effective amount of light can aid in regulating or accelerating the movement of teeth during orthodontic treatment with an orthodontic appliance, or regulating or accelerating bone remodeling during oral or maxillofacial bone remodeling with a functional appliance. The effective amount of light can be useful for reducing the amount of time an orthodontic appliance is worn during an orthodontic treatment, or that a functional appliance is worn during treatment for oral or maxillofacial bone remodeling. In other words, the effective amount of light can be useful for reducing a patient's orthodontic treatment time, in comparison to the orthodontic treatment time without administration of the effective amount of light. For example, according to the methods of the present invention, the application of light can reduce treatment time (e.g., wearing a functional appliance or an orthodontic appliance) by up to about 10%, about 20%, about 30%, about 40%, about 50%, about 60%, about 70%, about 75%, about 80%, about 85%, or about 90% of the treatment time. For example, administering light having a wavelength in the range of about 585 nm to about 665 nm (e.g., about 625 nm) can reduce the amount of time that a patient wears an orthodontic appliance or a functional appliance by about 5% to about 90%, for example, by about 5%, about 10%, about 20%, about 30%, about 40%, about 50%, about 60%, about 70%, about 75%, about 80%, about 85%, or about 90%. Administering light having a wavelength in the range of about 815 nm to about 895 nm (e.g., about 855 nm) can reduce the amount of time that a patient wears an orthodontic appliance or a functional appliance by about 5% to about 90%, for example, by about 5%, about 10%, about 20%, about 30%, about 40%, about 50%, about 60%, about 70%, about 75%, about 80%, about 85%, or about 90%.

In one or more embodiments, administering an effective amount of light with desired light characteristics results in an overall reduction in the amount of time necessary for treatment. For example, a treatment can comprise installing a functional appliance, the removal of the functional appliance, and the installation or removal of an orthodontic appliance. By combining the use of a functional appliance and an orthodontic appliance, the overall treatment time can be reduced. Furthermore, increased control on the bone remodeling and tooth movement can be delivered. This can be particularly advantageous during a patient's adolescent growth phase.

Administering light having a wavelength in the range of about 585 nm to about 665 nm (e.g., about 625 nm) can result in a rate of tooth movement that is about 5% to about 90% faster than the rate of tooth movement without the administration of light. For example, the rate of tooth movement can be about 5%, about 10%, about 20%, about 30%, about 40%, about 50%, about 60%, about 70%, about 75%, about 80%, about 85%, or about 90% faster than the rate of tooth movement without the administration of light.

Administering light having a wavelength in the range of about 815 nm to about 895 nm (e.g., about 855 nm) can result in a rate of tooth movement that is about 5% to about 60% faster than the rate of tooth movement resulting from the administration of light having a wavelength in the range of 585 nm to about 665 nm (e.g., about 625 nm). For example, the rate of tooth movement can be about 5%, about 10%, about 20%, about 30%, about 40%, about 50%, about 55%, or about 60% faster than the rate of tooth movement resulting from the administration of light having a wavelength in the range of 585 nm to about 665 nm (e.g., about 625 nm).

Administering light having a wavelength in the range of about 815 nm to about 895 nm (e.g., about 855 nm) can result in a rate of tooth movement that is about 5% to about 95% faster than the rate of tooth movement without the administration of light. For example, the rate of tooth movement can be about 5%, about 10%, about 20%, about 30%, about 40%, about 50%, about 60%, about 70%, about 75%, about 80%, about 85%, about 90%, or about 95% faster than the rate of tooth movement without the administration of light.

In one or more embodiments, in addition to light treatment, orthodontic treatments, particularly those that comprise the use of an orthodontic appliance (e.g., aligners such as INVISALIGN™), can exert forces on one or more teeth. This can result in a rate of tooth movement that is about 5% to about 80% faster than the rate of tooth movement without the exertion of heavy forces. For example, the exertion of a force in one or more teeth can increase the rate of tooth movement by about 5%, about 10%, about 20%, about 30%, about 40%, about 50%, about 60%, about 70%, about 75%, or about 80%. Forces can result in tooth-root resorption, bone resorption, inflammatory resorption of dentin, cementum resorption, or tissue inflammation In another embodiment, the exertion of a heavy force in one or more teeth can increase the rate of tooth movement by about 5%, about 10%, about 20%, about 30%, about 40%, about 50%, about 60%, about 70%, about 75%, or about 80%. Heavy forces can result in tooth-root resorption, bone resorption, inflammatory resorption of dentin, cementum resorption, or tissue inflammation.

In one or more embodiments, the administration of an effective amount of light can aid in reducing, preventing or minimizing tooth-root resorption when a heavy force is allowed to be exerted on one or more tooth. The effective amount of light can be useful for reducing the amount of tooth-root resorption as compared to when a force (e.g., from an orthodontic appliance) is allowed to be exerted on one or more tooth without administering the effective amount of light. For example, according to the methods of the present invention, the administration of light can reduce tooth-root resorption (e.g., apical root resorption) by up to about 1%, about 2%, about 3%, about 5%, about 7%, about 10%, about 15%, about 20%, about 25%, about 30%, about 40%, about 50%, about 60%, about 70%, about 80%, or about 90%, about 1% or more, about 2% or more, about 3% or more, about 5% or more, about 7% or more, about 10% or more, about 15% or more, about 20% or more, about 25% or more, about 30% or more, about 40% or more, about 50% or more, about 60% or more, about 70% or more, about 80% or more, or about 90% or more. Reducing tooth-root resorption, particularly while applying a force, can allow for a reduction of the amount of time for orthodontic treatment, or the amount of time that a patient wears an orthodontic appliance. Administering an effective amount of light can reduce the amount of time that a patient wears orthodontic appliances by about 5% to about 90%, for example, by about 5%, about 10%, about 20%, about 30%, about 40%, about 50%, about 60%, about 70%, about 75%, about 80%, about 85%, or about 90%.

In one or more embodiments, the administration of an effective amount of light can aid in reducing, preventing or minimizing tooth-root resorption when a heavy force is allowed to be exerted on one or more tooth. The effective amount of light can be useful for reducing the amount of tooth-root resorption as compared to when a heavy force is allowed to be exerted on one or more tooth without administering the effective amount of light. For example, according to the methods of the present invention, the administration of light can reduce tooth-root resorption (e.g., apical root resorption) by up to about 1%, about 2%, about 3%, about 5%, about 7%, about 10%, about 15%, about 20%, about 25%, about 30%, about 40%, about 50%, about 60%, about 70%, about 80%, or about 90%. Reducing tooth-root resorption, particularly while applying heavy forces, can allow for a reduction of the amount of time for orthodontic treatment, or the amount of time that a patient wears an orthodontic appliance. Administering an effective amount of light can reduce the amount of time that a patient wears orthodontic appliances by about 5% to about 90%, for example, by about 5%, about 10%, about 20%, about 30%, about 40%, about 50%, about 60%, about 70%, about 75%, about 80%, about 85%, or about 90%.

In one or more embodiments, administration of an effective amount of light can aid in reducing, preventing or minimizing bone resorption or inflammatory dentin or cementum resorption of the tooth root or periodontium. The effective amount of light can be useful for reducing bone resorption or inflammatory dentin or cementum resorption of the tooth root and periodontium, as compared to when a heavy force is allowed to be exerted on one or more teeth without administering the effective amount of light. For example, according to the methods of the present invention, the administration of light can reduce bone resorption or inflammatory dentin or cementum resorption of the tooth root or periodontium by up to about 1%, about 2%, about 3%, about 5%, about 7%, about 10%, about 15%, about 20%, about 25%, about 30%, about 40%, about 50%, about 60%, about 70%, about 80%, or about 90%. Reducing bone resorption or inflammatory resorption of dentin or cementum resorption of the tooth root or periodontium while exerting heavy forces can reduce the amount of time for orthodontic treatment, or amount of time that a patient wears an orthodontic appliance. Administering an effective amount of light can reduce the amount of time that a patient wears orthodontic appliances by about 5% to about 90%, for example, by about 5%, about 10%, about 20%, about 30%, about 40%, about 50%, about 60%, about 70%, about 75%, about 80%, about 85%, or about 90%.

In one or more embodiments, administration of the effective amount of light can aid in reducing, preventing or minimizing inflammation of tissue surrounding one or more teeth upon which heavy forces are or were exerted. The effective amount of light can be useful for reducing the amount of inflammation of tissue surrounding one or more teeth upon which heavy forces are or were exerted, as compared to when a heavy force is allowed to be exerted on one or more tooth without administering the effective amount of light. In one or more embodiments, according to the methods of the present invention, the administration of light can reduce inflammation of tissue surrounding one or more teeth upon which heavy forces are or were exerted by up to about 1%, about 2%, about 3%, about 5%, about 7%, about 10%, about 15%, about 20%, about 25%, about 30%, about 40%, about 50%, about 60%, about 70%, about 80%, or about 90%. Reducing inflammation of tissue surrounding one or more teeth upon which heavy forces are or were exerted while applying heavy forces can reduce the amount of time for orthodontic treatment, or amount of time that a patient wears an orthodontic appliance. Administering an effective amount of light can reduce the amount of time that a patient wears an orthodontic appliance by about 5% to about 90%, for example, by about 5%, about 10%, about 20%, about 30%, about 40%, about 50%, about 60%, about 70%, about 75%, about 80%, about 85%, or about 90%.

The light can be administered via the intra-oral apparatus in accordance with a treatment regimen. In one or more embodiments, when a functional appliance or an orthodontic appliance is used in combination with the intra-oral apparatus, the functional appliance or orthodontic appliance can be installed prior to administering the light via the intra-oral apparatus, the functional appliance or orthodontic appliance can be installed concurrently with administering the light via the intra-oral apparatus, the functional appliance or orthodontic appliance can be installed subsequent to administering the light via the intra-oral apparatus, or any combination thereof. In one or more embodiments, a functional appliance or an orthodontic appliance can be removed prior to administering the light via the intra-oral apparatus, the functional appliance or orthodontic appliance can be removed concurrently with administering the light via the intra-oral apparatus, the functional or orthodontic appliance can be removed subsequent to administering the light via the intra-oral apparatus, or any combination thereof. In one or more embodiments, a functional appliance or orthodontic appliance can be adjusted prior to administering the light via the intra-oral apparatus, the functional appliance or orthodontic appliance can be adjusted concurrently with administering the light via the intra-oral apparatus, the functional appliance or orthodontic appliance can be adjusted subsequent to administering the light via the intra-oral apparatus, or any combination thereof.

The functional appliance or orthodontic appliance can exert a force on one or more teeth of the patient in addition to or in lieu of the intra-oral apparatus exerting a force on one or more teeth. A force can be exerted (e.g., by the functional appliance and/or the orthodontic appliance subsequent to, concurrently with, or prior to the administration of light via the intra-oral apparatus. A force can be exerted subsequent to, concurrently with, or prior to initiation of the administration of light. A force can be exerted subsequent to, concurrently with, or prior to the initiation of a light treatment regimen involving the intra-oral apparatus. A force can be exerted subsequent to, concurrently with, or prior to the initiation of a light treatment session involving the intra-oral apparatus. In one or more embodiments, a force can be exerted one or more seconds, one or more minutes, one or more hours, one or more days or one or more weeks subsequent to administering the light and/or one or fewer days, one or fewer weeks, or one or fewer weeks subsequent to administering the light. The light can be administered by the intra-oral apparatus for any length of time. In one or more embodiments, a force is exerted one or more seconds, one or more minutes, one or more hours, one or more days or one or more weeks subsequent to initiating light administration and/or one or fewer days, one or fewer weeks, or one or fewer weeks subsequent to initiating light administration. In one or more embodiments, a force can be exerted one or more seconds, one or more minutes, one or more hours, one or more days or one or more weeks subsequent to ending light administration and/or one or fewer days, one or fewer weeks, or one or fewer weeks subsequent to ending light administration. The force can be, for example, a heavy force.

Light can be administered for any period of time before, during, or after the exertion of a heavy force. For example, light can be administered for about 1 minute, about 2 minutes, about 3 minutes, about 5 minutes, about 10 minutes, about 15 minutes, about 20 minutes, about 30 minutes, about 45 minutes, about 1 hour, about 90 minutes, about 2 hours, about 3 hours, about 4 hours, or about 6 hours prior to, during, or after the exertion of a force, such as a heavy force. In one or more embodiments, light is administered for about 5 minutes to about 10 minutes. In one or more embodiments, light is administered at any amount of time prior to, during, or after the initiation of the exertion of a force. For example, light can be administered about 1 minute, about 2 minutes, about 3 minutes, about 5 minutes, about 10 minutes, about 15 minutes, about 20 minutes, about 30 minutes, about 45 minutes, about 1 hour, about 90 minutes, about 2 hours, about 3 hours, about 4 hours, about 6 hours, about 12 hours, about 1 day, about 36 hours, about 2 days, about 3 days, about 4 days, about 1 week, about 2 weeks, or about 1 month prior to, during, or after the initiation of the exertion of a force. In one or more embodiments, light is administered for about 5 minutes to about 10 minutes.

Administering light prior to initiating or exerting a force, as described herein, can be part of a pretreatment regimen. In one or more embodiments, however, no such pretreatment occurs and the functional appliance or orthodontic appliance exerts a force prior to any light being administered. The functional appliance or orthodontic appliance can exert a force, for example, at one or more seconds, one or more minutes, one or more hours, one or more days or one or more weeks prior to administering the light and/or one or fewer days, one or fewer weeks, or one or fewer weeks prior to administering the light. Thus, a follow-up treatment of light can be provided after the exertion of the force. In one or more embodiments, a force is exerted during the administration of light, or at one or more stages of the administration of light.

In one or more embodiments, the functional appliance or orthodontic appliance exerts the force at the same region as the region that is administered with light. In other embodiments, the functional appliance or orthodontic appliance exerts the force on a region that is different from the region that is administered with light. In one or more embodiments, allowing the functional appliance or orthodontic to exert a force on a region other than the region administered with light can result in allowing a force to be exerted to a region that is administered with light.

In one or more embodiments, the dosage or effective amount of light has a density that ranges from about 24 J/cm² to about 200 J/cm², and has a wavelength in the range of about 585 nm to about 665 nm, or about 815 nm to about 895 nm. Administration of light having a wavelength in the range of about 585 nm to about 665 nm can be useful in the present methods, in one or more embodiments, for promoting bodily movement of teeth or minimize tipped movement of teeth, or both. Administration of light having a wavelength in the range of 815 nm to about 895 nm, can also be useful in the present methods, for example, for increasing the velocity of teeth through the patient's bone. In some other embodiments, an effective dosage of light can have any of the light characteristics as described anywhere herein. Teeth in a region of the patient's maxillary or mandibular alveolar bone to which light is not administered can be used as an anchor to facilitate movement of teeth in the selected region. Light is administered directly to a specific region of the patient's mouth, e.g., the patient's alveolar soft tissue, using the intra-oral apparatus, as described herein. In some embodiments where light is administered directly to a specific region, the light reaches the specific region without first reflecting from another region.

In one or more embodiments, the present methods comprise administering to a patient in need thereof, via an intra-oral apparatus, an effective amount of light having a first wavelength to a selected first region of the patient's mouth (e.g., a first region of the alveolar soft tissue), and further comprise administering, via the intra-oral apparatus, an effective amount of light having a second wavelength to a selected second region of the patient's mouth (e.g., a second region of the alveolar soft tissue). In one or more embodiments the effective amount of light having a first wavelength is a repetitive dosage. In another embodiment the effective amount of light having a second wavelength is a repetitive dosage. Regions other than alveolar soft tissue can receive the first or second wavelength of light. In one or more embodiments, the effective amount of light can be in the range of 24 J/cm² to 200 J/cm². The first wavelength can be in the range of about 585 nm to about 665 nm, and the second wavelength can be in the range of about 815 nm to about 895 nm. In other embodiments, an effective amount of light can have any light characteristics as described anywhere herein.

In one or more embodiments, the methods further comprise installing the intra-oral apparatus and/or a secondary appliance (such as, for example, a functional appliance or an orthodontic appliance), removing the intra-oral apparatus and/or a secondary appliance, or adjusting the intra-oral apparatus and/or a secondary appliance. In other embodiments, the methods comprise administering light via the intra-oral apparatus until orthodontic treatment is complete. Orthodontic treatment can be deemed complete after appointments with an orthodontic specialist are completed, after the movement of one or more teeth has been stabilized to remain in the substantially same position, e.g., plus or minus about 1 to about 3 millimeters of a specific position, without the aid of any type of orthodontic appliance, or during a passive stage of orthodontic treatment as described in greater detail herein. Light can be administered to the region before, during, after, or any combination thereof, a secondary appliance is installed, adjusted, or removed. The secondary appliance can be applied, adjusted, or removed before, during, after, or any combination thereof, the application of light via the intra-oral apparatus. In one or more embodiments, a force, such as a heavy force, can be exerted when the orthodontic appliance is installed or adjusted, or for a period of time following such installation or adjustment.

As described herein, the speed of tooth movement, e.g., through the bone, or the quality of that movement (e.g., “bodily” or “tipped” movement) can be regulated by administration of light. In one or more embodiments the present methods are useful for effecting bone regeneration, which can occur concurrently with the present methods. Bone regeneration can be enhanced by administering light according to the present methods. Where orthodontic treatment is performed, light can be administered before, during or after orthodontic treatment using the intra-oral apparatus. The light can be emitted from the intra-oral light-therapy apparatus in any manner described herein. Bone regeneration can include bone growth or bone resorption. This can include osteoblast or osteoclast activation. Tooth movement can require osteoclastic and osteoblastic activity. In one or more embodiments, the administration of light according to the present methods stimulates osteoclasts or osteoblasts and, accordingly, stimulates osteoclastic and osteoblastic activity. The administration of light can increase the rate of tooth movement that can accompany bone remodeling.

For example, the present methods, in one or more embodiments for regulating tooth movement, can also comprise applying, adjusting or removing a tooth mask or other oral mask. The mask can be coupled to, disposed within, or otherwise part of the intra-oral apparatus. In one or more embodiments, one or more of the panels of the intra-oral apparatus can comprise a mask. In other embodiments, the mask is separate from the intra-oral apparatus but is configured to contact the intra-oral apparatus when the patient is wearing the intra-oral apparatus. A tooth mask can be applied or removed prior to, during, or after the administration of light. Light can be administered to a region before, during, after, or any combination thereof, an oral mask or tooth mask is applied, adjusted, or removed. In one or more embodiments, one or more of a patient's teeth, or other region of the patient's mouth, can be at least partially covered with a mask that can block at least some of the light. A mask can block one or more wavelengths of light. In one or more embodiments, the mask can completely block one or more wavelength of light, and in other embodiments, the mask can reduce the amount or intensity of light reaching the teeth, or other region of the patient's mouth. In one or more embodiments, the intensity of the light administered to the teeth, or other region of the patient's mouth, can be zero, or can be less than the intensity of the light emitted from a light source.

In accordance with another aspect of the invention, the present methods, in one or more embodiments for tooth-movement regulation, can regulate the bone regeneration. For example, the present methods can increase the rate of bone regeneration. In one or more embodiments, bone regeneration can facilitate tooth-movement regulation, for example, can increase the velocity or quality of movement, or can stabilize tooth movement. For example, bone regeneration can occur prior to, during or following tooth movement. Bone regeneration can include bone growth, bone strengthening or bone resorption. For example, during bone regeneration, bone mineral density (BMD) can increase, bone volume (BV) can increase, bone mineral content (BMC) can increase, and the ratio of bone volume to total volume (BV/TV) or bone density can increase. Higher BV/TV can indicate denser bone, where less bone regeneration can occur, which is desirable after tooth movement has occurred to enhance the stability of teeth. Specifically, teeth move more slowly through denser and more mineralized bone, and maintain their position longer than in less dense bone. Teeth are therefore less likely to relapse and move back to their original, misaligned state. In this manner, light treatment can increase the quality of the bone or, more specifically, increase the mineral density of the bone to prevent relapse after orthodontic treatment. In some such embodiments, the light treatment occurs after force is applied to the one or more teeth of the patient (i.e., when force is no longer being applied to the patient's teeth). In one or more embodiments, the patient wears an orthodontic appliance during such light treatment. In another embodiment, the patient does not wear an orthodontic appliance during such light treatment.

Other examples of parameters that can be affected during bone regeneration can include trabecular bone surface, bone quality, osteoclastic activity (e.g., osteoclast and preosteoclast counts), and bone resorption. Light treatment can enhance existing cellular processes. Bone regeneration can occur in any bone tissue or oral region. For example, bone regeneration can occur in a portion or all of a maxillary alveolar bone, in mandibular alveolar bone, or around one or more teeth. In one or more embodiments, bone regeneration can occur around one or more teeth, which can include a periodontium. In one or more embodiments, the region around one or more teeth can be plus or minus about 1 mm, about 2 mm, or about 3 mm from the surface of the teeth.

In one or more embodiments, light treatment according to the present methods can also result in treating or preventing jaw osteonecrosis. Accordingly, the present methods are useful for treating or preventing jaw osteonecrosis. Accordingly, the invention further provides methods for treating or preventing jaw osteonecrosis, comprising administering an effective amount of light to a patient, wherein the effective amount of light is irradiated from the emitter of an apparatus of the present invention. In one or more embodiments, at least a portion of the apparatus contacts the alveolar soft tissue when the light is administered. The methods optionally comprise allowing a heavy force to be exerted on one or more teeth of the patient, who is in need thereof. When applicable, light can be administered before, during or after the heavy force is exerted Treating or preventing jaw osteonecrosis can comprise reversing osteonecrosis through the use of light treatment according to the methods described herein. Jaw osteonecrosis can occur with respect to any bone tissue. For example, jaw osteonecrosis, can occur with respect to a portion or all of a maxillary alveolar bone, mandibular alveolar bone, or one or more teeth. In one or more embodiments, methods for treating or preventing jaw osteonecrosis further comprise administering to the patient an effective amount of vitamin D.

In one or more embodiments, light treatment according to the present methods can also result in reducing, minimizing, or preventing tooth-root resorption, bone resorption, inflammatory resorption of dentin or cementum resorption, or inflammation of tissue. Accordingly, the present methods are useful for reducing, minimizing, or preventing tooth-root resorption, bone resorption, inflammatory dentin or cementum resorption, or inflammation of tissue. Accordingly, the invention further provides methods for reducing, minimizing, or preventing tooth-root resorption (e.g., apical root resorption), bone resorption, inflammatory dentin or cementum resorption, or inflammation of tissue, comprising administering an effective amount of light to a patient, wherein the effective amount of light is irradiated from the emitter of an apparatus of the invention, and, optionally, allowing a heavy force to be exerted on one or more teeth of the patient, who is in need thereof. In one or more embodiments, at least a portion of the apparatus contacts the alveolar soft tissue when the light is administered. When applicable, light can be administered before, during, or after the heavy force is exerted. Such light-treatment methods can be useful in combination with heavy forces applied to one or more teeth.

In one or more embodiments, light treatment regulates tooth movement during an alignment phase of orthodontic treatment. In another embodiment, light treatment regulates tooth movement during a phase other than the alignment phase of orthodontic treatment, including, but not limited to, a gap or space closure phase or orthodontic treatment. In yet another embodiment, light therapy regulates tooth movement following the alignment phase of orthodontic treatment.

In one or more embodiments, the region to which light is administered is any oral tissue, such as soft tissue or bone tissue, including the alveolar soft tissue and, in one or more embodiments, the alveolar mucosa. In one or more embodiments, the oral tissue is that on which oral surgery was performed. In one or more embodiments, the oral tissue includes one or both of hard and soft tissue at the site of oral surgery within the patient's oral cavity. The present methods are useful for at least one of treating tissue and promoting soft tissue healing after one or more of oral surgery, maxillofacial surgery, craniofacial surgery and orthognathic surgery. The oral surgery can include periodontal surgery; surgery for orthodontic purposes; surgery for non-orthodontic purposes; placement of one or more dental implants; surgery relating to bone grafts; surgery relating to oral cancer; abnormal cell growth, or a tumor; surgery for tooth removal; gingival surgery; and surgery relating to connective tissue grafts, gingival grafts, or other soft tissue grafts. More specifically, the oral surgery can include surgery for tooth removal for orthodontic reasons and surgery to remove one or more teeth for non-orthodontic reasons, including, but not limited to, tooth removal because of one or more of the following conditions: supernumerary teeth, ectopic teeth (e.g., in which the canine tooth is impacted in the patient's palate), impacted wisdom teeth, periodontal disease, fractured or otherwise traumatized teeth, failing infected root canal treated teeth, unrestorable teeth, or infected teeth. The oral tissue can include one or more of: a portion or all of tissue supporting one or more teeth, the gums (i.e., gingiva), the alveolar soft tissue, a maxillary alveolar bone, mandibular alveolar bone, or one or more teeth. Accordingly, the invention further provides methods for treating oral tissue after oral surgery, comprising administering an effective amount of light to a patient, wherein the effective amount of light is irradiated from the emitter of an apparatus of the invention. In one or more embodiments, the treating comprises healing.

In one or more embodiments, light administration to the oral tissue begins no more than about 24 hours from performance of the oral surgery (including, for example, placement of a dental implant). In one or more embodiments, light administration to the oral tissue begins no more than about 6 hours from performance of the oral surgery (including, for example, placement of the dental implant). In one or more embodiments, light administration to the oral tissue begins no more than about 1 hour from performance of the oral surgery (including, for example, placement of the dental implant). In one or more embodiments, light is administered to the oral tissue in one or more treatment sessions. In one or more embodiments, the light is administered to the oral tissue for about 1 minute to about 10 minutes during the treatment session. In one or more embodiments, the light is administered for about 6 minutes during the treatment session. In one or more embodiments, the light is administered for about 3 minutes during the treatment session. In one or more embodiments, the light is administered to the patient during one treatment session per day. In one or more embodiments, the light is administered to the patient during two treatment sessions per day.

In one or more embodiments, at least a portion of the apparatus contacts, when the light is administered, a region of the patient's alveolar soft tissue on which surgery was performed. In one or more embodiments, the method also comprises allowing a heavy force to be exerted on one or more teeth of the patient, who is in need thereof. When applicable, light can be administered before, during or after the heavy force is exerted. The present methods are also useful for increasing the rate of oral-tissue healing following oral surgery. Accordingly the invention further provides methods for increasing the rate of oral-tissue healing following oral surgery, comprising administering an effective amount of light to a patient, wherein the effective amount of light is irradiated from the emitter of an apparatus of the invention. In one or more embodiments, at least a portion of the apparatus contacts, when the light is administered, a region of the patient's alveolar soft tissue on which surgery is intended to be performed. In one or more embodiments, the method also comprises allowing a heavy force to be exerted on one or more teeth of the patient, who is in need thereof. When applicable, light can be administered before, during or after the heavy force is exerted.

In one or more embodiments, the methods further comprise performing oral surgery on the oral tissue. The oral surgery can be performed prior to or subsequent to the administration of light treatment according to the present methods. In one or more embodiments, the region of light administration can be the alveolar bone or the alveolar soft tissue. In one or more embodiments, the administration occurs for about 20 minutes. In one or more embodiments, the wavelength of administered light is about 625 nm. In one or more embodiments, the wavelength of administered light is about 850 nm. In one or more embodiments, light is administered at an intensity of about 100 mW/cm². In one or more embodiments, the light can be administered following oral surgery, prior to oral surgery, or during oral surgery. In one or more embodiments, the light is administered to tissue on which oral surgery was performed, prior to oral surgery or during oral surgery. In one or more embodiments, the tissue will be or is in need of healing, e.g., as a result of the oral surgery. In one or more embodiments, the invention relates to methods for treating nerve damage or numbness, which can occur as a result of oral surgery (e.g., on a patient's jaw). In one or more embodiments, the invention relates to methods for treating or preventing periodontitis, comprising administering an effective amount of light to a patient, wherein the effective amount of light is irradiated from the emitter of an apparatus of the invention.

In one or more embodiments, the invention relates to methods for treating a site of oral surgery, comprising administering an effective amount of light to a patient, wherein the effective amount of light is irradiated from the emitter of an apparatus of the invention. In one or more embodiments, the treating comprises healing. In one or more embodiments, the invention relates to methods for healing oral tissue adjacent to one or more dental implants, for example, endosseous dental implants. More specifically, the methods can comprise healing oral tissue at a site of surgical placement of one or more dental implants, in which the site includes one or both of hard tissue and soft tissue. Healing of the oral tissue can include one or both of accelerating the growth of bone between a bore drilled into the jaw for placement of the dental implant and the surface of the dental implant and accelerating osseo-integration of endosseous dental implants. The method for healing surgical site oral tissue comprises administering an effective amount of light to a patient, wherein the effective amount of light is irradiated from the emitter of an apparatus of the invention. In one or more embodiments, at least a portion of the apparatus contacts the alveolar soft tissue when the light is administered. In one or more embodiments, the methods further comprise allowing a heavy force to be exerted on one or more teeth of the patient who is in need thereof. When applicable, light can be administered before, during or after the heavy force is exerted. In one or more embodiments, these methods can be performed according to the teachings disclosed herein for the methods for regulating tooth movement.

In one or more embodiments, the present methods can further comprise applying a substance to a region, such as an oral region, e.g., the alveolar soft tissue, or in the proximity of a region, before, during, or after the administration of light. The substance can be applied before, during, or after the intra-oral apparatus is within the mouth of the patient. In one or more embodiments the methods do not comprise applying a substance to a region, or in the proximity of a region, before, during, or after the administration of light, or before, during, or after the exertion of heavy forces. In some instances, a substance can already occur at a region naturally. The substance can enhance or inhibit the effects of the light administration. In one or more embodiments, the substance can be a visible-light- or infrared-light-absorbing substance, such as a dye. One or more light characteristics, such as wavelength, can be selected in response to the presence or application of the substance.

Although methods for treating a site of oral surgery have been described herein, in one or more embodiments, as increasing or accelerating a rate of oral tissue healing following the oral surgery, in other embodiments, it can be desirable to delay healing of the oral tissue at the site of the oral surgery. For example, an orthodontic treatment program can comprise a first phase, during which a patient's teeth are aligned and leveled, and a second phase in which gaps or spaces between teeth are closed (e.g., through retraction of one or more anterior teeth and/or mesial movement of one or more posterior teeth). In some treatment programs, one or more of the patient's teeth (e.g., one or more premolars in a crowded dental arch) are extracted before an orthodontic appliance is installed and before the first phase of the orthodontic treatment. Light therapy treatment, as described in various embodiments herein, can, in some cases, stimulate and accelerate healing of oral tissue at the site of extraction. The accelerated healing can include an increased rate of bone growth as the extraction site (e.g., tooth socket) is healed. The higher bone density at the extraction site (compared to the expected bone density of a normal rate of healing in the absence of light therapy), however, might not be preferable for patients whose orthodontic treatment program includes the second phase for gap or space closure, because the higher bone density can slow down movement of tooth roots that is needed for the gap or space closure. In other words, the tooth socket at the extraction site can include denser and more mature bone, than would otherwise occur in the absence of the light therapy for tooth movement during the first, alignment, phase, and thus slow the resorption of bone and movement of tooth roots during the gap closure phase of orthodontic treatment. Accordingly, in a method according to an embodiment, a barrier implant 2798 (see, e.g., FIG. 114) is implanted at an extraction site E following extraction of the tooth. In one or more embodiments, the barrier implant 2798 is implanted immediately after the tooth is extracted, such as during the same oral surgery session. As such, the barrier implant 2798 is also implanted before light therapy is administered. The barrier implant 2798 is configured to prevent accelerated healing at the extraction site E as a result of the light therapy administration during an alignment phase of orthodontic treatment. The barrier implant 2798 can be, for example, a resorbable collagen plug. The collagen plug 2798 can be configured to resorb as new bone is formed at the extraction site E, and such that the new bone is formed more slowly than the accelerated healing that would otherwise occur because of the light therapy administration.

One aspect of the invention provides for a light treatment kit comprising an intra-oral light-therapy apparatus as described herein and instructions for use in the present methods. The kit can further comprise a light source that is separate from the intra-oral light-therapy apparatus. The separate light source and/or the light sources of the intra-oral light-therapy apparatus can be removable and disposable, so that they can be easily replaced after a given amount of use. In one or more embodiments, a light-therapy apparatus and separate light sources can be individually packaged or can be packaged together. The separate light source can operate in combination with the light sources of the intra-oral light-therapy apparatus to aid in light administration. The separate light sources can emit light in any manner described herein and can further have any wavelength of characteristic described herein.

The kit can also comprise a programmable controller as described herein. The kit can further comprise any components useful for the controller to operate. For example, the kit can comprise a component to power the controller or the intra-oral light-therapy apparatus. The kit can also comprise a component that allows the controller to operably connect with an intra-oral light-therapy apparatus.

The kit can also comprise software, an algorithm, or one or more computer readable media that can provide instructions to a controller. The software, algorithm, or set of computer readable media can be provided on a memory medium. The memory medium can be a removable or portable, such as a CD, USB flash drive, or external hard drive

The kit can be conveniently packaged and can be commercially available. The kit can also comprise written instructions for use or maintenance of items therein.

As described herein, a light therapy apparatus according to embodiments of the invention can be electronically linked, or paired, with an external device, such as a mobile phone, including smartphones (e.g., an iPhone® or an Android™ based device), personal digital assistance, computer, tablet, portable electronic device, or the like. In this manner, the apparatus can be configured for at least one of wireless uni-directional or wireless bi-directional communication with the external device, such as via a Bluetooth® or other wireless connection. In one or more embodiments, the apparatus (e.g., apparatus 2500) is configured to transmit data associated with a patient's compliance with a prescribed treatment program, during which the patient is to use the apparatus to administer light therapy to the patient's teeth, to the external device. As such, in one or more embodiments, a light therapy system comprises the external device. The external device can be used, for example, by a dental practitioner to receive, store, and analyze the patient compliance data, in addition to other patient information, as described herein. The external device in one or more embodiments, is used by a patient to track progress through a light therapy regimen.

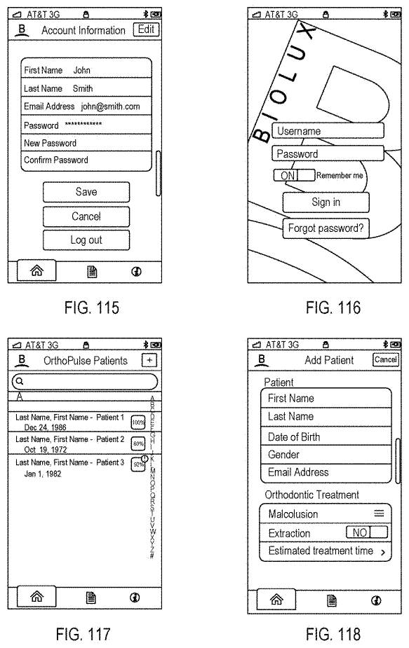

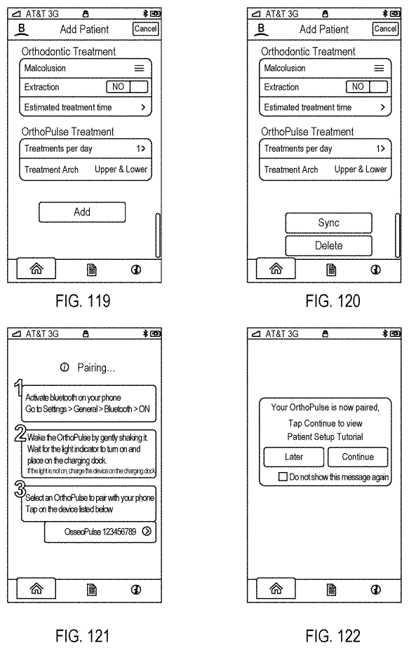

Referring to FIGS. 115-124, the external device can be configured to execute code to at least one of receive, store, or process the patient compliance data transmitted by the light therapy apparatus. For example, an application including the executable code can be loaded onto the external device. The executable code can be configured to display one or more screens on the external device to enable the dental practitioner to enter information associated with one or more patients and access the patient compliance data.

The external device can be configured for the practitioner (or a designated compliance administrator) to create a new account. For example, as shown in FIG. 115, the external device can require entry of the practitioner's name, an email address, and a password (and can comprise password reset capabilities). As shown in FIG. 116, the external device can be configured to require entry of log-in credentials to access the application, such as via a long-in screen, to maintain security of the patient information. As shown in FIG. 117, the external device can be configured to display a list of patients for whom patient information has been entered onto the external device.

As shown in FIGS. 118-120, the external device can be configured to display a screen via which information associated with a new patient or an existing patient can be added to the external device. For example, external device can be configured to receive patient information such as the patient's name, date of birth, gender, contact information (e.g., an email address). The external device can also be configured to receive information associated with an orthodontic treatment program for the patient. For example, the external device can be configured to receive information about the patient's malocclusion, whether the patient's orthodontic treatment comprises extracting a tooth, and the patient's estimated treatment time and/or treatment prescription, e.g., number of aligners and the amount of time per aligner. In one or more embodiments, the external device is configured to receive information about the patient's light therapy treatment program, including the number of treatment sessions to be administered per day, and the arch or arches (i.e., upper, lower, or upper and lower arches) to which light therapy is to be administered during the treatment session. In one or more embodiments, the external device is accessed by a clinician to alter a patient's light therapy treatment program. The external device in another embodiment is used to program the light therapy apparatus, for example, the number of light emitters to use for a particular treatment, and/or the number of distinct light emitter zones to use. As described herein, a zone can include one or more light emitters (e.g., one or more LEDs).

As shown in FIG. 121, the external device is configured to be paired with a light therapy apparatus, e.g., a light therapy apparatus according to an embodiment described herein. In one or more embodiments, the external device is configured to display instructions on a pairing screen for pairing the external device and the light therapy apparatus. The external device can be configured to display a confirmation indicating whether or not the external device and the light therapy apparatus were successfully paired, as shown in FIG. 122. The external device can also be configured to provide a tutorial, for example, to assist the practitioner with entering the patient information as shown in FIGS. 96-98. The external device can be configured to be paired with the light therapy apparatus before or after the patient information is entered into the external device. After the external device and light therapy apparatus are paired, the external device can receive the patient compliance data from the light therapy apparatus.

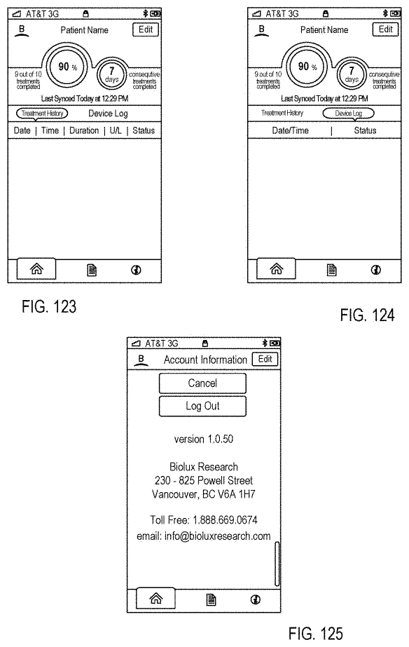

The external device can be configured to display at least a portion of the patient compliance data. For example, as shown in FIGS. 123 and 124, the external device can be configured to display a number of treatments sessions completed by and prescribed for the patient (e.g., “9 out of 10”) and/or a percentage of the total number of prescribed treatments sessions completed by the patient (e.g., 90%). The external device can be configured to display a number of consecutive treatment sessions completed by the patient (e.g., “7 days of consecutive treatments completed”). The external device can be configured to display the last incidence (e.g., date and time) patient compliance data was received from the light therapy apparatus. In other words, the external device can display when the patient compliance data stored on the external device was last synced with the patient compliance data stored on the light therapy apparatus. As shown in FIG. 120, the external device can be configured to synchronize patient compliance data with the light therapy apparatus, if, for example, such synchronization was not already initiated by the light therapy apparatus itself.

The clinician in one or more embodiments reassesses the patient's treatment protocol depending on the compliance data. For example, the clinician determines based on the compliance data whether to advance the patient to another aligner, to turn on different light emitter zones during light therapy, to increase the number of light emitters turned on in a zone during a light therapy session, etc.

The external device can also be configured to display a log of the patient's usage of the light therapy apparatus, including a listing of one or more of the date a treatment session was administered using the light therapy apparatus, the time of the treatment session, the duration of the treatment session, whether the treatment session was administered with respect to the patient's upper or lower arch, and a status of the treatment session or light therapy apparatus. As shown in FIG. 124, the external device can also be configured to display a log associated with the light therapy apparatus itself, including a status of the apparatus and a date and time of the status. The status log can, for example, list the light therapy apparatus' status history indicating each time the apparatus was in one or more of the apparatus statuses (or states), such as sleep, ready, charge, communication, or error statuses, described herein. Finally, as shown in FIG. 125, the external device can be configured to provide a log out option to exit the application. The external device can also be configured to display contact information, such as for a manufacturer of the light therapy apparatus, as shown in FIG. 125.

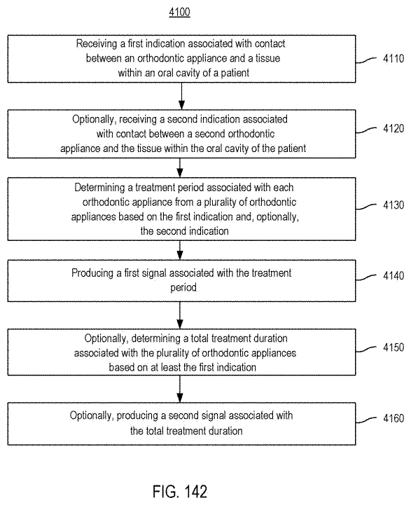

As shown in FIG. 142, a method 4100 according to an embodiment can comprise determining a rate by which a patient should transition from use of one orthodontic appliance applied to one or more teeth of the patient to use of another orthodontic appliance applied to one or more teeth of the patient. For example, an orthodontic treatment regimen for the patient can comprise using a plurality of orthodontic appliances, one at a time and in a predetermined or orthodontic-prescribed sequence. Stated another way, the regimen can provide for sequentially removably coupling each orthodontic appliance from the plurality of orthodontic appliances to one or more teeth of the patient. More specifically, in one or more embodiments, each orthodontic appliance can be an aligner from a plurality, or set, of aligners configured to be removably coupled to the teeth of the patient in a predetermined order or sequence. The plurality of aligners can comprise at least one substantially transparent aligner. Each aligner of the plurality of aligners can be substantially transparent. In other embodiments, the plurality of orthodontic appliances can comprise any other suitable orthodontic appliance described herein. The time period during which the orthodontic appliance (e.g., a first aligner) is coupled to the patient's teeth is referred to with respect to this method as a treatment “period” during the treatment regimen. The patient need not wear the orthodontic appliance and/or have the orthodontic appliance installed on the patient's teeth continuously during the period. For example, for removable orthodontic appliances, the period could span several days or weeks, during which the patient can remove the appliance for periods (e.g., to brush their teeth, etc.).

Generally, a first phase of the method 4100 comprises assessing a patient's individual rate of tooth movement during a period at which a portion of the orthodontic appliances from the plurality of orthodontic appliances are sequentially applied to the patient's teeth based on a predetermined schedule of when the orthodontic appliance is to be removed and replaced by the next orthodontic appliance in the sequence (also referred to herein as “switching” appliances), and a second phase of the method 4100 comprises determining a modified or accelerated rate (compared to the predetermined schedule) of orthodontic appliance switching for the remaining orthodontic appliances from the plurality of orthodontic appliances in the sequence. The modified or accelerated rate of orthodontic appliance switching can be based on the patient's natural rate of tooth movement and/or a rate of tooth movement during use of the portion of orthodontic appliances from the plurality of orthodontic appliances. As such, the modified or accelerated rate of orthodontic appliance switching (i.e., the period determined for the patient) is specific to the patient and can optimize the patient's tooth movement during the orthodontic treatment and reduce the patient's overall orthodontic treatment time.

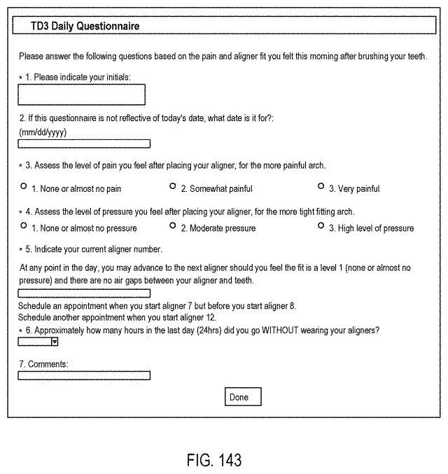

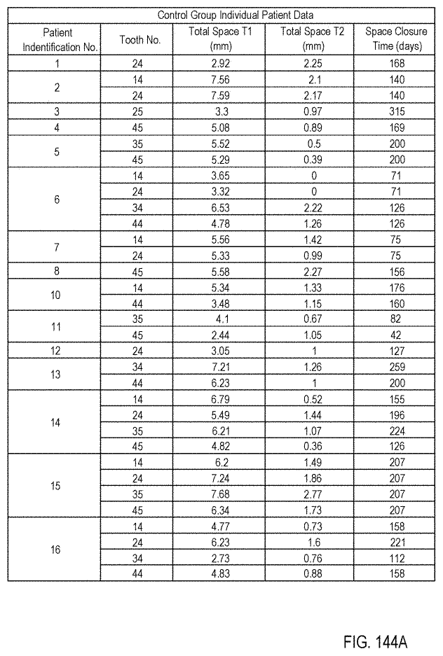

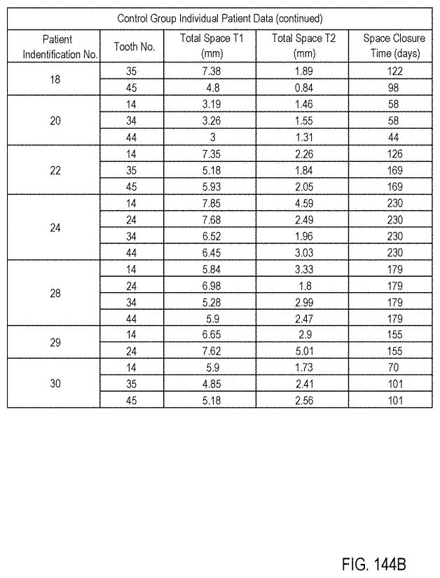

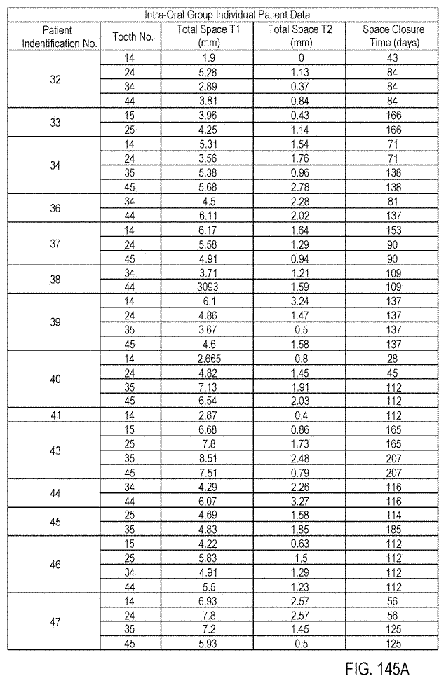

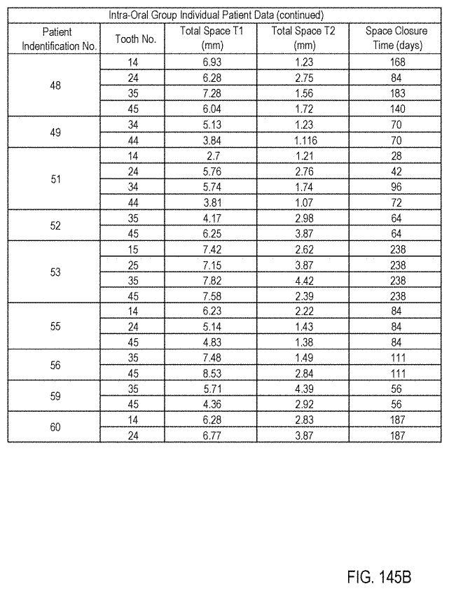

At 4110, the method 4100 comprises receiving an indication associated with a contact between the orthodontic appliance and a tissue within an oral cavity of the patient. More specifically, the indication can be associated with one or more sensations caused by at least one of a pressure exerted by the orthodontic appliance removably within the oral cavity of the patient and/or a perceived pain associated with the orthodontic appliance that is or was coupled to the patient's teeth or can include an assessment of the indication by the patient. For example, in one or more embodiments, the indication includes the number of air gaps present between the teeth and orthodontic appliance, when the orthodontic appliance is coupled to the patient's teeth, and are perceived by the patient through visual assessment. In a further embodiment, the indication includes the aforementioned assessment of air gaps and one or more additional sensations. The sensation can include one or both of pressure and pain, and in one or more embodiments the one or more sensations (e.g., pressure and pain) are self-assessed by the patient. The self-assessment in one or more embodiments includes the assessment of pain on a scale of 1 to 3, where “1” indicates no pain or almost no pain. “2” indicates some pain and “3” indicates much pain. In another embodiment, the self-assessment includes the assessment of pressure exerted by the orthodontic appliance on a scale of 1 to 3, where “1” indicates no pressure or almost no pressure, “2” indicates moderate pressure and “3” indicates a high level of pressure (see, e.g., FIG. 143). In one or more embodiments, the receiving comprises receiving a pressure indication associated with the pressure exerted by the orthodontic appliance on the tissue within the oral cavity and a pain indication associated with the orthodontic appliance being coupled to the teeth of the patient. In other embodiments, the receiving comprises receiving an indication associated with a reduction in and/or absence of a pressure exerted by the orthodontic appliance and/or a reduction in and/or absence of a pain indication associated with the orthodontic appliance being coupled to the teeth of the patient.

The indication can be received, for example, at device external to the orthodontic appliance (also referred to herein as the “external device”), such as a mobile phone, including smartphones (e.g., an iPhone® or an Android™ based device), personal digital assistance, computer, tablet, portable electronic device, or the like. In this manner, for example, an orthodontist (or dental practitioner) can, remotely from the patient, access data associated with the received indication(s). As such, the orthodontist can review and/or evaluate the data and prescribe an accelerated or otherwise modified rate of orthodontic appliance switching for a remainder of the orthodontic treatment program including the remainder of the plurality of orthodontic appliances. Such remote access to the indication data allows the patient and orthodontist to avoid having multiple office visits. In one or more embodiments, as described in more detail below, the orthodontist and/or the patient can execute code to perform at least a portion of the evaluation of the received indication(s) data and/or determining a modified or accelerated orthodontic appliance switching based on the evaluation.

The indication can be received daily, or at another suitable interval. For example, pain, pressure and/or fit in one or more embodiments, is assessed on a daily basis by the patient and/or orthodontist. In one or more embodiments, the indication is received each day during the course of treatment with the orthodontic appliance or plurality thereof. In one or more embodiments, the indication is received each day for a predetermined number of days. For example, in one or more embodiments, the predetermined number of days is from about four days to about ten days. In another example, in one or more embodiments, the predetermined number of days is six days. Although the indication is received each day for the predetermined number of days, data included in the indication can vary over the course of the predetermined number of days. In other words, and as an example only, an indication received on day one of the predetermined number of days can include an indication of a sensation of pressure but no pain, while an indication received on day two of the predetermined number of days can include an indication of a sensation of pressure and a sensation of pain. However, in other embodiments, the indication does not vary over the course of predetermined number of days (e.g., pain, pressure and fit are assessed on each day).

In one or more embodiments, the indication is input by the patient, as described in more detail herein. For example, a patient can input into an electronic device his or her answers to a series of questions related to the patient's use of the orthodontic appliance. Stated another way, the external device can be configured to receive data associated with one or more patient inputs into the patient's electronic device, including patient responses to an electronic questionnaire, such as the questionnaire shown in FIG. 143 (e.g., to assess pain, pressure and/or fit as described herein). Referring to FIG. 143, the patient can input his or her initials (or other identifying information) and, optionally, indicate the date of orthodontic appliance usage for which the questionnaire is being completed. The patient can indicate a level of pain felt by the patient after applying the orthodontic appliance to one or more of the patient's teeth. For example, the indication can be a level of pain and/or pressure felt by the patient after applying an aligner to one or more of the patient's teeth. In one or more embodiments, the indication is based on the more painful arch of the upper or lower arches. Example pain indications include (1) none or almost no pain, (2) some pain, and (3) much pain. The patient can indicate a level of pressure felt by the patient after applying the orthodontic appliance to one or more of the patient's teeth. For example, the indication can be a level of pressure felt by the patient after applying the aligner to one or more of the patient's teeth. In one or more embodiments, the indication is based on the tighter fitting arch of the upper or lower arches. Example pressure indications include (1) none or almost no pressure, (2) moderate pressure, and (3) high level of pressure. In one or more embodiments, the indication relates to the number of air gaps present between the teeth and the aligner, scored on a scale of 1-3, where 1 indicates no air gaps. The patient can indicate the orthodontic appliance that is currently being applied to one or more of the patient's teeth, or that was applied to the one or more of the patient's teeth for the date of orthodontic appliance usage for which the questionnaire is being completed. For example, the patient can indicate an aligner number for a particular aligner from a plurality, or set, of aligners. The patient can indicate approximately how many hours in the last day (or 24 hour period) that the patient did not wear any orthodontic appliance from the plurality of orthodontic appliances. The external device can also optionally be configured to receive, via patient inputs to the questionnaire using the electronic device, additional comments from the patient.

In one or more embodiments, the orthodontic appliance is a first orthodontic appliance (e.g., an aligner) of the plurality of orthodontic appliances, the predetermined number of days is a first predetermined number of days, and/or the indication is a first indication. In such embodiments, the method 4100 optionally comprises, at 4120, receiving a second indication associated with a sensation experienced by the patient and caused by a second orthodontic appliance from the plurality of orthodontic appliances. The indication can be the number of air gaps present between the patient's teeth and aligner, a sensation such as pressure or pain caused be the aligner, or a combination of the three. More specifically, the second indication can be associated with at least one of a pressure exerted by the second orthodontic appliance removably applied to the patient's teeth and/or within an oral cavity of the patient or a perceived pain associated with the second orthodontic appliance applied to the patient's teeth. In one or more embodiments, the receiving comprises receiving a pressure indication associated with the pressure exerted by the second orthodontic appliance and a pain indication associated with the second orthodontic appliance. In other embodiments, the receiving comprises receiving an indication associated with a reduction in and/or absence of a pressure exerted by the orthodontic appliance and/or a reduction in and/or absence of a pain indication associated with the orthodontic appliance being coupled to the teeth of the patient. The second indication can be received each day for a second predetermined number of days.

At 4130, the method 4100 comprises determining a treatment period associated with each orthodontic appliance from the plurality of orthodontic appliances based on the indication (e.g., the first indication and/or the second indication). In one or more embodiments, the treatment period is determined based on the first indication, the second indication or both the first and second indication. For example, where the first indication is associated with one or more air gaps between an aligner and a patient's teeth or one or more sensations, e.g., pain or fit (pressure), the treatment period for the first orthodontic appliance of the plurality of orthodontic appliances is the number of days until pain is assessed as no pain or almost no pain, pressure is assessed as no pressure or almost no pressure, and no air gaps are present. In a further embodiment, where a plurality of orthodontic appliances are used in a treatment method, the treatment period for each of the respective plurality of orthodontic appliances is the number of days until pain is assessed as no pain or almost no pain, pressure is assessed as no pressure or almost no pressure, and no air gaps are present. In one or more embodiments, a weighted transition factor can be determined based on the indication for each day and the number of hours during which the orthodontic appliance has or had been within the oral cavity of the patient (e.g., applied to the one or more teeth of the patient). For example, the indication received by the external device can include an indication of the number of hours during which the orthodontic appliance was applied to the one or more teeth of the patient. Such information can be input by the patient, as described in more detail herein. In one or more embodiments, a tooth movement factor is determined based on the indication for each day. The tooth movement factor is associated, at least in part, with an amount of movement of one or more of the patient's teeth. In one or more embodiments, a weighted light factor is determined based on the indication for each day and a light indication associated with whether the patient has received a light therapy during the period when the orthodontic appliance has or had been within the oral cavity of the patient. The weighted light factor can include and/or be associated with any other parameter associated with the light therapy, such as the duration of the light therapy per day, the wavelength of the light therapy, the power of the light used during the light therapy or the like.

At 4140, the method 4100 comprises producing a signal associated with the treatment period. For example, the signal (e.g., a first signal) can be produced after the treatment period is determined. In one or more embodiments, the signal is produced by the external device. The signal can be an electronic signal, including, but not limited to, a text message, a visual indicator, a graphical depiction. In one or more embodiments, the signal is sent to an electronic device associated with the patient, including, for example, a mobile phone, including smartphones (e.g., an iPhone®, a Google® device, or an Android™ based device), personal digital assistant, computer, tablet, portable electronic device, or the like.

At 4150, the method 4100 optionally comprises determining a total treatment duration associated with the plurality of orthodontic appliances based on the indication. The total treatment duration is the total duration of the treatment regimen during which the plurality of orthodontic appliances is removably coupled to the patient's teeth.

At 4160, the method 4100 also optionally comprises producing a second signal associated with a total treatment duration. For example, the second signal can be an electronic signal configured to be sent to the patient's device. The second signal can be configured to indicate to the patient the total period of time (duration) during which the patient sequentially has or will use the plurality of orthodontic appliances for their prescribed orthodontic treatment. As such, the second signal can also be configured to indicate when the patient's orthodontic treatment, or at least the portion of the orthodontic treatment including use of the sequence of orthodontic appliances, will end or has ended.

In one or more embodiments, the external device can be configured to execute code to at least one of receive the indication(s), determine the treatment period, and produce the signal(s). For example, an application including the executable code can be loaded onto the external device. The executable code can be configured to perform the following algorithm:

T=(N×D)−(A×D)

T2=(N×M)−(A×M)

where:

N=the total number of orthodontic appliances in the plurality of orthodontic appliances for the patient's arch with the highest number of prescribed orthodontic appliances;

D=in number of days, the frequency of orthodontic appliance switching pursuant to the predetermined schedule;

M=in number of days, a natural rate of orthodontic appliance switching after an evaluation period during the predetermined number of days;

A=the number of the orthodontic appliance in use by the patient with respect to the orthodontic appliance's placement in the sequence of the plurality of orthodontic appliances (e.g., orthodontic appliance number n in a sequence of 1 . . . n . . . 12 orthodontic appliances);

T=a time remaining for orthodontic appliance switching based on the predetermined schedule of orthodontic appliance switching;

T2=a time remaining for orthodontic appliance switching based on the modified or accelerated rate of orthodontic appliance switching.

The executable code can be configured to determine a percentage reduction in orthodontic treatment time for the patient using the modified or accelerated rate of orthodontic appliance switching based on the following algorithm: (T−T2)/T.

The method optionally comprises administering light therapy when one or more orthodontic appliances of the plurality of orthodontic appliances are sequentially coupled to the one or more teeth of the patient. For example, the first orthodontic appliance can be applied to one or more of the patient's teeth, at a first time and for a first time period. Light is administered to one or more of the patient's teeth when the first orthodontic appliance is applied to the one or more of the patient's teeth during at least a portion of the first time period. For example, light can be administered according to any suitable method described herein, including at any suitable wavelength, intensity, or for any suitable duration, as described herein. In one or more embodiments, a light therapy apparatus is disposed about or over a portion of the first orthodontic appliance when the light is administered. In one or more embodiments, the light therapy apparatus is configured to detect a number, within the sequence of the plurality of orthodontic appliances, of an orthodontic appliance applied to one or more of the patient's teeth. For example, the light therapy apparatus can be configured to detect the orthodontic appliance's number using radio frequency identification (“RFID”). In such embodiments, for example, the light therapy apparatus can comprise an RFID reader in at least one of an intra-oral housing or an extra-oral housing of the light therapy apparatus. In another example, the light therapy apparatus comprises an optical bar code reader configured to read an optical bar code of the orthodontic appliance, which can indicate the orthodontic appliance's number.

A method according to an embodiment for aligning one or more of a patient's teeth. The methods comprise disposing a first orthodontic appliance within an oral cavity of a patient such that the orthodontic appliance is removably coupled to the teeth of the patient. The first orthodontic appliance can be any suitable orthodontic appliance described herein, including, but not limited to, an aligner. The methods comprise determining a time period specific to the patient. The time period during which an orthodontic appliance (e.g., the first orthodontic appliance, such as a first aligner) is coupled to the patient's teeth is referred to with respect to this method as a “period” during a treatment regimen. The method comprises maintaining the first orthodontic appliance within the oral cavity for the period. Although the orthodontic appliance is described herein as being maintained within the oral cavity during the period, in one or more embodiments, the patient need not wear the orthodontic appliance and/or have the orthodontic appliance installed on the patient's teeth absolutely continuously during the period. For example, for removable orthodontic appliances, the period could span several days or weeks, during which the patient can remove the appliance for brief periods (e.g., to brush their teeth, etc.) and then reinstall the appliance within the oral cavity. After the period, a second orthodontic appliance is disposed within the oral cavity such that the second orthodontic appliance is removably coupled to the teeth of the patient.

The method can comprise coupling orthodontic appliances from the plurality of orthodontic appliances, in sequence, until alignment of one or more of the patient's teeth is achieved. For example, in one or more embodiments, the patient can sequentially dispose a first, a second, a third . . . up to an nth orthodontic appliance (where n is any suitable integer, for example, from four to thirty) from the plurality of orthodontic until alignment is achieved. In use, for example, the patient can wear the first orthodontic appliance for a period of time, then replace the first orthodontic appliance with the second orthodontic appliance, which is worn for a subsequent period of time, then replace the second orthodontic appliance with a third orthodontic appliance, which is worn for a subsequent period of time, and so on with repeatedly replacing (e.g., removing the most recently worn appliance and installing the next appliance in the sequence) orthodontic appliances until alignment is achieved. In one or more embodiments, alignment of one or more teeth of one or both of the upper or lower arches is achieved when the one or both of the upper arch LII score or the lower arch LII score is ranges from zero (0) mm to less than one (1) mm. In other words, the patient's teeth can be determined to be in alignment when the patient's LII score for the arch being measured is less than 1 mm. An LII score of zero (0) can indicate that the teeth are perfectly aligned.

In one or more embodiments, each orthodontic appliance from the plurality of orthodontic appliances is disposed within the oral cavity of the patient such that each orthodontic appliance is removably coupled to the patient's teeth (also referred to herein as being “worn”) by the patient, in order, for a period of time. The period of time can be, for example, a few days (e.g., 2, 3, 4, 5, or 6 days) or one or more weeks (e.g., 1, 2, or 3 weeks). The period of time that one orthodontic appliance from the plurality of orthodontic appliances is worn by the patient can be different than a period of time that another orthodontic appliance from the plurality of orthodontic appliances is worn by the patient. In other words, each orthodontic appliance from the plurality of orthodontic appliances can be worn by the patient for a period time that is independent or irrespective of a period of time for which another orthodontic appliance from the plurality of orthodontic appliances is worn by the patient. For example, the first orthodontic appliance can be worn for a first period of time, the second orthodontic appliance can be subsequently worn for a second period of time that is less than or greater than the first period of time in duration. In one or more embodiments, a third orthodontic appliance is worn subsequent to the second orthodontic appliance for a third period of time that is less or greater in duration than at least one of the first period of time or the second period of time. In other embodiments, the third orthodontic appliance is worn subsequent to the second orthodontic appliance for a third period of time that is substantially equal in duration to (e.g., plus or minus up to 5% of) the first period of time or the second period of time. Additionally, a first portion or subset of the plurality of orthodontic appliances can be worn for a substantially equal period of time (e.g., plus or minus up to 5%), and second portion or subset of the plurality of orthodontic appliances can be worn for one or more periods of time different than the period of time the first portion of plurality of orthodontic appliances is worn.

In one or more embodiments, a method for coupling orthodontic appliances from a plurality of orthodontic appliances can comprise administering a light therapy using any of the light therapy apparatuses shown and described herein. In one or more embodiments, a method for changing orthodontic appliances (replacing a first orthodontic appliance with a second, unused orthodontic appliance) can comprise administering light therapy using any of the light therapy apparatuses shown and described herein. For example, in one or more embodiments, a method can comprise disposing a first orthodontic appliance within an oral cavity of a patient such that the first orthodontic appliance is removably coupled to the teeth of the patient. The first orthodontic appliance can be any suitable orthodontic appliance from one or more removable orthodontic appliances as described herein. The method further comprises administering a light therapy when the first orthodontic appliance from the set of orthodontic appliance is within the oral cavity of the patient. The light therapy can be administered using any of the light therapy apparatuses shown and described herein.

A period specific to the patient is determined. As described herein, the period is the time period during which an orthodontic appliance from the set of orthodontic appliances is to be worn according to a treatment regimen. The period can be determined using any of the methods described herein. For example, in one or more embodiments, the period can be determined based on a light indication associated with the light therapy administered. In other embodiments, as described herein, the period can be determined based on an indication associated with the reduction of the pain, a reduction of a sensation, an assessment of whether air gaps between the teeth and orthodontic appliance exist and/or the reduction the pressure associated with the first orthodontic appliance. The first orthodontic appliance is maintained within the oral cavity for the period.

The method further comprises disposing a second orthodontic appliance within the oral cavity of the patient after the period such that the second orthodontic appliance is removably coupled to the teeth of the patient.

One or more embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code or algorithm) can be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks, carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code disclosed herein.

One or more embodiments and/or methods described herein can be performed by software (executed on hardware), hardware, or a combination thereof. Hardware modules can include, for example, a general-purpose processor (or microprocessor or microcontroller), a field programmable gate array (FPGA), and/or an application specific integrated circuit (ASIC). Software modules (executed on hardware) can be expressed in a variety of software languages (e.g., computer code), including C, C++, Java®, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

While various embodiments of the invention have been described herein, it should be understood that they have been presented by way of example only, and not limitation. For example, although apparatus (e.g., apparatus 2100) have been described herein as including a gyroscope, in other embodiments, an apparatus can comprise any suitable mechanism for detecting tilt and/or spatial orientation of the apparatus such that the mechanism can help determine whether the apparatus is in an upright or inverted position.

In another example, although apparatus (e.g., apparatus 2100) have been illustrated and described herein as including an intra-oral housing configured to be positioned within the patient's mouth for administration of light to one of the upper jaw and the lower jaw at a time, in other embodiments, an apparatus comprises an intra-oral housing comprising upper and lower flanges, each including an LED array coupled to or embedded therein. In this manner, the apparatus is configured to concurrently administer light therapy with respect to each of the upper and lower jaws.

Although the bite pad 2514 is described herein as having a thickness that varies from the anterior portion (thinner) to the posterior portion (thicker), in other embodiments, the anterior portion of the bite pads shown and described herein can be thicker than that of the posterior portion. Moreover, in one or more embodiments, a thickness of any of the bite pads shown herein can vary along any direction, for example, from a lingual (or inside portion) to the buccal (or cheek-side) portion.

Although the mouthpiece 2510 is shown as including a notch 2530, a first groove 2532 and a second groove 2534, in other embodiments, any of the mouthpieces shown and described herein can comprise any suitable geometric features and/or combinations of materials to produce the desired flexibility for placement of the light array. For example, in one or more embodiments, the mouthpiece 2510 and any of the mouthpieces shown and described herein can comprise a series of notches along the upper portion of the flanges 2522, 2524, a series of circumferential perforations about the buccal portion (i.e., the cheek-side) and/or the lingual (i.e., inside) portion, or the like.

### Orthodontic Appliance Compliance Systems, Devices, and Methods

Described herein are devices, systems, and methods for measuring compliance with use of an orthodontic appliance (e.g., an orthodontic aligner or another orthodontic appliance disclosed herein) or a light therapy apparatus, as well as methods for making and using the same. A system according to some embodiments of the invention is configured to monitor a patient's (or user's) compliance with use of an orthodontic appliance pursuant to an orthodontic treatment program. Compliance devices can measure a user's use of any suitable orthodontic appliance. For example, compliance devices can measure, detect or monitor a user's use of one or more aligners (e.g., orthodontic aligners), braces (e.g., a wire and bracket system), light therapy apparatuses, such as those described herein, or any other suitable orthodontic appliances. Compliance devices can be located in the mouth of a user, such as bonded directly to a patient's tooth. In other embodiments, the compliance devices are included in or attached to an orthodontic appliance, such as mounted to an aligner, light therapy apparatus, etc. In some embodiments, a compliance devices can be configured to communicate data to or receive data from a data collection device. For example, the compliance device can communicate data to a transceiver of an orthodontic appliance (such as an aligner or a light therapy apparatus). In other embodiments, the compliance device can communicate data to an intra-oral or extra-oral probe that collects data from the compliance device upon being brought in close proximity with the device. In some embodiments, data collection devices can additionally offload data to an external computational device (e.g., a processor of a computer, mobile phone, etc.)

### Systems

Systems for monitoring user compliance with an orthodontic appliance treatment plan can comprise a compliance monitoring device, an orthodontic appliance (such as an aligner or any suitable orthodontic appliance), a data collection device, and optionally a light therapy apparatus. In some embodiments, a compliance monitoring device comprises a component such as a sensor, power source, transceiver, memory device, processor, or power management system. The compliance monitoring device can be configured to be attached to the tooth of a user, or be at least partially embedded in an orthodontic appliance or to the surface of an orthodontic appliance. The compliance monitoring device can be configured to detect an input (such as temperature, pressure, change in capacitance, etc.) that informs whether an orthodontic appliance is present in a user's mouth.

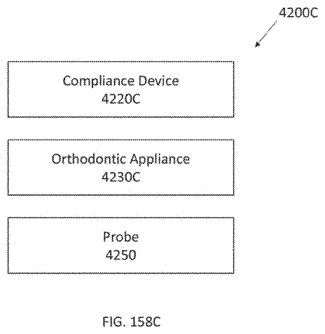

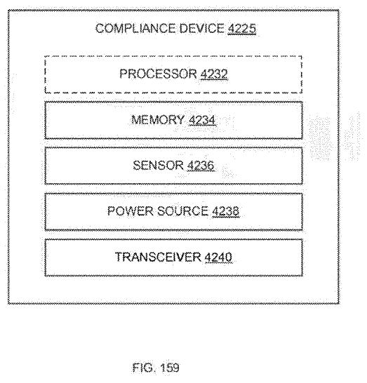

FIG. 158A illustrates a system 4200A useful for monitoring a patient's use of an orthodontic appliance during an orthodontic treatment program, according to some embodiments. More particularly, the system 4200A is configured to detect, monitor, assess, evaluate, and/or determine, or any combination thereof, placement or usage of an orthodontic appliance within a patient's mouth. The system of FIG. 158A comprises an orthodontic appliance 4230A, a compliance device 4220A, and a data collection device 4260. In some embodiments, the data collection device 4260 can be a light therapy apparatus or a probe. The compliance device can be disposed within the patient's mouth, and can be coupled to the patient's tooth, or at least partially embedded in or attached to the orthodontic appliance. The system can be configured to detect whether the orthodontic appliance is being used by (e.g., is disposed within the mouth of) the user in a manner consistent with or otherwise pursuant to a prescribed or predetermined treatment program. For example, the system 4200A is useful for assessing whether the user has disposed one or more orthodontic appliances within the user's mouth for (or for at least) a predetermined duration.

In some embodiments, such as the illustrative embodiment depicted in FIG. 158B, the system 4200B can comprise a compliance monitoring device 4220B (also referred to herein as “compliance device”), an orthodontic appliance 4230B, and a light therapy apparatus 4210. The compliance monitoring device 4220B can be configured to be disposed within a patient's mouth (or oral cavity). In some embodiments, the compliance monitoring device 4220B is coupled, and in some of these embodiments, removably coupled, to an orthodontic appliance 4230B that is configured to be at least partially disposed within the patient's mouth such that the compliance monitoring device 4220B is disposed within the mouth. In some embodiments, the compliance monitoring device 4220B is configured to be disposed within the patient's mouth independently of the orthodontic appliance. For example, the compliance monitoring device 4220B can be coupled, e.g., permanently or removably, including using any method or material for bonding described herein including with respect to bonding of an appliance such as bracket and wires, to a tooth of the patient. In some embodiments, an outer surface of the patient's tooth is etched to help facilitate bonding of the compliance device 4220B to the tooth's etched surface.

In some embodiments, such as the illustrative embodiment depicted in FIG. 158C, a system 4200C can comprise a compliance monitoring device 4220C, an orthodontic appliance 4230C, and a data collection device such as probe 4250. The compliance monitoring device 4220C can be coupled to a patient's tooth, or be at least partially embedded in or attached to an orthodontic appliance. The probe 4250 can be configured to communicate with the compliance device 4220C. For example, the probe can comprise a receiver to receive data from the compliance device 4220C. In some embodiments, the probe 4250 can comprise a processor, to process data received from the compliance device; a transmitter, to transmit information to the compliance monitoring device; and/or memory to store data received from the compliance device.

Compliance Monitoring Device

FIG. 159 depicts a schematic representation of a compliance monitoring device as described herein. As depicted there, a compliance monitoring device 4225 can comprise a sensor 4236, a memory 4234, and a transceiver 4240, and optionally a power source 4238 and/or a processor 4232. In some embodiments, the compliance monitoring device 4225 comprises a flexible circuit board. In some embodiments, one or more of the components of the compliance monitoring device 4225 can be formed or included on the flexible circuit board. In some embodiments, one or more (or all) components of the compliance monitoring device 4225 (including the flexible circuit board) can be partially or fully disposed inside a casing of the compliance device 4225, as described in more detail herein.

Processor

The processor can be, for example, a general-purpose processor, a microprocessor, a microcontroller, a field programmable gate array (FPGA), an application specific integrated circuit (ASIC), and/or the like. For example, in some embodiments, the processor is a microcontroller such as an Atmel® tinyAVR® microcontroller. The memory can include Read-Only Memory (ROM), Random-Access Memory (RAM), electrically erasable programmable read-only memory (EEPROM), and/or any suitable non-transitory processor-readable medium. The memory can be included in the processor, or can be included in a separate component configured to be in electrical communication with the processor and/or an external electronic device.

Sensor

The sensor is configured to receive an input. The input can be, for example, a physiological parameter of the patient (e.g., temperature, pulse, respiration, etc.), force or pressure, capacitance change, optical parameter, or other suitable input. The sensor can be or include any suitable sensor or combination of sensors, including those sensors described herein. In some embodiments, the sensor can be configured to send a signal including or associated with the input to the memory, and the memory can store the input as data.

The input (or data) can be used, as described in more detail herein, to determine whether an orthodontic appliance is disposed within the patient's mouth and/or within a predetermined distance (e.g., from about 0 mm to about 10 mm, from about 1 mm to about 10 mm, from about 1 mm to about 50 mm, from about 1 mm to about 1 cm, from about 50 mm to about 1 cm) of the compliance device, thereby determining or at least implicating a presumption that the orthodontic appliance is at least partially disposed within the patient's mouth. Disposition of the orthodontic appliance within (or at least partially within) the patient's mouth, as detected by the sensor, determines or at least supports an inference that the orthodontic appliance is in use within the patient's mouth, e.g., pursuant to a prescribed or predetermined orthodontic treatment program.

The sensor of the compliance monitoring device can be configured to detect from within the patient's mouth an input. For example, the compliance monitoring device can be configured to detect a physiological parameter of the patient. The physiological parameter can include, for example, a parameter or value associated with the patient's body temperature, pulse, respiration (e.g., rate, oxygen exchange), or the like. In use, for example, the sensor can detect from within the patient's mouth, e.g., the temperature of the patient's oral cavity, when the compliance monitoring device is disposed within the patient's mouth. The sensor can include, for example, a thermistor (e.g., a negative temperature coefficient (NTC) thermistor), a resistance temperature detector (RTD), a thermocouple, a semiconductor based sensor (e.g., a diode with temperature sensitive voltage or current parameters), and/or the like, or a combination of the foregoing. In some embodiments, the sensor configured to detect the physiological parameter is coupled, permanently or removably, to the orthodontic appliance. In this manner, detection by the sensor of the physiological parameter, as described herein, is indicative that that orthodontic appliance is at least partially disposed within the patient's mouth.

In some embodiments, the sensor is or includes a force sensor or pressure sensor (e.g., a force reactive resistor). In this manner, for example, the sensor can detect a force or pressure at a contact point between the sensor and an oral tissue (e.g., tooth, gum, alveolar mucosa or other suitable oral tissue), e.g., such as can be generated when the orthodontic appliance with the compliance device coupled thereto is disposed within the patient's mouth. In some embodiments, the sensor can detect a force or pressure consistent with the orthodontic appliance being removed or otherwise not positioned in the patient's mouth. In other embodiments, the sensor can detect a force or pressure generated by the orthodontic appliance being disposed in contact with the compliance device (e.g., in an embodiment in which the compliance device is coupled to the patient's tooth or other oral tissue before the orthodontic appliance is disposed in the mouth). In some embodiments, a force sensor can comprise a switch (such as a switch encapsulated in a coating, such as silicone) configured to be activated when it is depressed, such as when an aligner is seated over the compliance monitoring device. The switch can be coupled to a timer that records the amount of time the aligner is in and/or out of the patient's mouth when the switch is activated. For example, in some embodiments, the switch can activate a timer, and record the amount of time an orthodontic appliance is in a user's mouth (e.g., in increments of 5 minutes, 10 minutes, 15 minutes, 30 minutes, 60 minutes, etc.). Additionally or alternatively, the switch can activate a timer and record the amount of time an orthodontic appliance is not in a user's mouth (e.g., in increments of 5 minutes, 10 minutes, 15 minutes, 30 minutes, 60 minutes, etc.). In other embodiments, the switch can operate to deactivate a timer when it is depressed, such that the timer records an amount of time an orthodontic appliance is not disposed in a patient's mouth. In these embodiments, the timer turns off when an orthodontic appliance is seated over the compliance monitoring device, and turns on when an orthodontic appliance is removed. In some embodiments, in order to distinguish between application of one or more forces not associated with the presence of an orthodontic appliance (e.g., brushing teeth, eating, tongue or cheek movement, etc.), the force sensor can be activated only after the switch is depressed for a particular amount of time (e.g., 3 seconds, 5 seconds, 10 seconds, or any suitable amount of time) to begin recording an amount of time an appliance is in place (or, alternatively, to stop recording an amount of time that an appliance is not in place).

In some embodiments, the sensor of the compliance device can be or include a magnetic sensor or a sensor otherwise configured to detect magnetism. For example, in an embodiment in which the compliance device is coupled to a tooth independently of and prior to disposal of an orthodontic appliance within the patient's mouth, the orthodontic appliance can comprise a magnet configured to activate a silicon semiconductor of the compliance device when the magnet is disposed within a predetermined distance of the compliance device (e.g., a distance of about 1 cm or less, about 0.5 cm or less, between about 0.1 cm and about 1 cm, between about 0.1 cm and about 0.5 cm, or between about 0.5 cm and about 1 cm.). In some embodiments, the sensor can comprise a switch configured to be open or closed when the magnet (of the orthodontic appliance) is disposed within the predetermined distance of the switch. In some embodiments, a magnet can be bonded to the tooth, and the compliance device can be configured to detect the magnet's magnetism when disposed over and/or within proximity (e.g., a distance of about 1 cm or less, about 0.5 cm or less, between about 0.1 cm and about 1 cm, between about 0.1 cm and about 0.5 cm, or between about 0.5 cm and about 1 cm) of the magnet.

In some embodiments, the sensor is configured detect a change in capacitance (or the presence of capacitance change), e.g., of a semiconductor of the compliance device, in response to the orthodontic appliance being positioned within a predetermined distance (e.g., a distance of about 1 cm or less, about 0.5 cm or less, between about 0.1 cm and about 1 cm, between about 0.1 cm and about 0.5 cm, or between about 0.5 cm and about 1 cm) of the compliance device. The change in capacitance can be detected in any suitable manner, including via a sensor as described herein. In some embodiments, the sensor of the compliance device is a light sensor or optical sensor (e.g., a photodiode or proximity detector), as described herein.

The sensor can be in electrical communication with (or is electrically coupled to) the processor. In this manner, the sensor can be configured to detect the input (e.g., a physiological parameter such as temperature, force, pressure etc.), for example, pursuant to instructions executable by the processor. Such instructions can specify, for example, the frequency at which the sensor detects the input, the duration of which the sensor detects the input, whether the sensor is off or on, or the like. The sensor is configured to be in electrical communication with (or is electrically coupled to) the memory. In this manner, data associated with the detected input can be transmitted from the sensor to the memory and stored therein for later retrieval.

In some embodiments, the sensor of the compliance device is communicably coupled to the processor and configured to detect and/or determine input (e.g., temperature, force/pressure, etc.) related data during use of the compliance device when the compliance device is disposed in the patient's mouth. For example, the sensor and/or the processor can individually or collectively be configured to detect or determine any suitable input-related data including, but not limited to, instantaneous temperature of the patient, an average temperature of the patient over a predetermined time period, a moving temperature average for a predetermined time period, a rate of temperature change, an amount of time a sensor detected a certain temperature, and/or the like. In other embodiments, the sensor and/or the processor can individually or collectively be configured to detect or determine input-related data including, but not limited to, instantaneous force or pressure, average force or pressure, a rate of force or pressure change, an amount of time a force or pressure is applied, and/or the like. Such input-related data can be used to determine (e.g., by the processor, the light therapy apparatus, the probe, another electronic device such as an external processor, or a clinician/physician) whether or when the orthodontic appliance is disposed in the patient's mouth, and if so, calculate for how long, i.e., the duration of an instance of disposition within the mouth, a total duration of disposition within the mouth over a period of time, or the like, and optionally assess or compare the determination (e.g., via the processor, the light therapy apparatus, the probe, another electronic device, or a clinician/physician) with respect to a prescribed treatment program to assess the patient's compliance with orthodontic appliance usage prescribed pursuant to the treatment program.

Clock

In some embodiments, the compliance monitoring device comprises a clock or timer. The clock can be included in the processor or memory, or can be a separate component configured to be in electrical communication with the processor, the sensor, and/or the memory. In this manner, for example, the processor and/or the memory can be configured to associate with the detected input (e.g., the physiological parameter, pressure, light detection, magnetic field, semiconductor activation, switch opening or closing) date and/or time data determined or indicated by the clock, for example, where the date and/or time data correspond to the date and/or time that the input was detected by the sensor and/or to the date and/or time that data associated with the input were stored in the memory. The clock can also be configured to measure a length of time that a sensor is active or inactive. The clock can also be configured to assess, e.g., via the processor, whether a predetermined time interval has elapsed.

Power Source

The power source of the compliance monitoring device is configured to provide power to components of the compliance monitoring device including, but not limited to, the processor, memory, sensor and transceiver. The power source can be or include any suitable battery, including any suitable rechargeable battery. The power source can be a microbattery. The power source can be, for example, a lithium-ion battery, a nickel-metal hydride battery, a lithium-ion polymer battery, a sodium ion battery, a silver-zinc battery, a supercapacitor, a coin cell or button cell battery, and/or the like. In some embodiments, as described in more detail herein, the power source is rechargeable without partial or full removal of the compliance monitoring device from within the patient's mouth. For example, the power source can be chargeable using light, such as light transmitted by a light therapy apparatus, as described below. In other embodiments, the power source can be self-sustaining (e.g., has sufficient power to maintain the compliance device for the length of its use). In other embodiments, the power source comprises a non-rechargeable battery. In some of these embodiments, the non-rechargeable battery has an estimated life span of about 1 year, about 2 years, about 3 years, about 1 year to about 3 years, about 2 years to about 3 years, or more. Additionally or alternatively, the non-rechargeable battery can have an energy storage capacity of about 1 milliampere-hour and a maximum discharge rate of about 1 milliamp. In some embodiments, the non-rechargeable battery can have an energy storage capacity of about 5 milliampere-hour or of between about 1 milliampere-hour and about 5 milliampere-hour.

In some embodiments, the compliance monitoring device is a low-power device. For example, the compliance monitoring device can have a target power usage of about 0.1 mircowatts to about 0.3 microwatts when the compliance device is on and/or is in sensing mode. The compliance monitoring device can have a maximum target power usage of about 1.0 milliwatt to about 3.0 milliwatts during communication with a probe or other external device. In some embodiments, the compliance monitoring device has a target power usage of 1.0 microampere at 3.0 volts or 3 microwatts when the compliance device is on and/or is in the sensing mode.

Transceiver

The compliance device can also comprise a transceiver configured to receive and transmit signals. For example, a transceiver can transmit information associated with a detected input (e.g., physiological parameter, force, pressure, or any other detectable input) to a separate device. For example, the transceiver of the compliance device can transmit a signal indicative of data to a probe, a light therapy apparatus, a device coupled to an orthodontic appliance, or any other suitable data collection device. The transceiver of the compliance device can be in communication with and can transmit information from any of the components of the compliance device, such as the processor, sensor, or memory. For example, the sensor can collect compliance data (e.g., the amount of time an orthodontic appliance was within the mouth of a patient) and store the data in memory, and the transceiver can retrieve the data from memory and transmit the data to another device (e.g., via the processor).

In some embodiments, the transceiver can also be configured to send a signal to the light therapy apparatus or probe, which signal includes or is indicative of data associated with the input data and corresponding time and/or date data stored in the memory. The stored input data and corresponding time and/or date data can be, for example, data stored in the memory after a prior transmission of data from the transceiver to the light therapy apparatus or probe, or data stored in the memory for a predetermined period of time.

The transceiver can also be configured to receive information from external devices or processors (e.g. a light therapy apparatus, a probe, a computational device, or any other suitable data communication device). For example, the transceiver can receive a signal that includes instructions associated with monitoring patient compliance with the orthodontic treatment program. For example, the signal can include instructions for how frequently the sensor is to sense or detect (or attempt to sense or detect) the input (e.g., the physiological parameter, such as the temperature) or the duration of sensing or detecting (or attempting to sense or detect) the input (e.g., at a particular instance of detection) by the sensor.

The transceiver can communicate with an external device in any suitable manner. In some embodiments, the transceiver can receive or transmit data wirelessly via radiofrequency signals, optical signals, magnetic signals, or any other suitable signal. The transceiver of the compliance monitoring device can be, for example, a light transceiver or a light emitter. For example, in some embodiments, the transceiver is or comprises a light emitting diode (“LED”). The transceiver can be an LED that is operable, for example, as a photodiode light sensor. In some embodiments, the transceiver is configured for optical communication (e.g., via light) of an indication of the input (e.g., physiological parameter such as temperature, force/pressure, etc.) data and/or corresponding time and/or date data to the light therapy apparatus, and also for receiving information (e.g., operating instructions) from an external device (e.g., a probe, light therapy apparatus, computational device, other communication device, etc.) and/or for receiving light from an external device such as the light therapy apparatus to generate electricity to charge the battery, supercapacitor, or the like, as described herein. The light transceiver can be configured to transmit data (e.g., input data, time data, and/or date data) from the sensor or the memory, whether directly or via the processor, and/or the like. As disclosed herein, in some embodiments, the light transceiver is disposed in the proximity of, e.g., adjacent to, or is at least partially disposed within an optically transparent portion of a casing of the compliance monitoring device.

Although the compliance device is shown and described herein as comprising the transceiver, in other embodiments, a compliance device can comprise a transmitter and/or a receiver as separate components, a receiver without a transmitter, or a transmitter without a receiver.

In some embodiments, the light transceiver of a compliance monitor is operably coupled to (or is couplable to) a power source. In some embodiments, the light transceiver can be configured to charge (or recharge) the power source (e.g., battery, supercapacitor, or the like) based on at least a portion of the received light from the light therapy apparatus, as described herein. The compliance monitoring device can be configured to be recharged by the light therapy apparatus without removal of either the compliance monitoring device or the orthodontic appliance from the patient's (or user's) mouth. In other embodiments, the compliance monitoring device can be configured to be recharged by the light therapy apparatus with an orthodontic appliance removed from the mouth of the patient. In some embodiments, the compliance monitoring device optionally comprises a power management device. The power management device can, for example, be configured to control the transfer of energy (e.g., via voltage and/or current regulation, voltage and/or current level conversion, voltage and/or current scaling, inversion, or the like) from the light transceiver (e.g., with respect to the received light energy) to the power source, for example, and vice versa, to prevent the power source from being damaged during charging.

Casing

As described herein, the compliance monitoring device can be present in a casing (or housing) that can be configured to partially, e.g., substantially, or fully enclose one or more components of the compliance device. The casing can be composed of any material suitable for being disposed in the user's mouth. In some embodiments, the casing is hard and durable, e.g., for embodiments in which the compliance device is to be mounted directly to a patient's tooth. For example, the casing material can comprise a biocompatible dental composite resin. The biocompatible dental composite resin, for example, is readily bonded to tooth enamel, and thus is particularly suitable for embodiments in which the compliance device is bonded directly to the patient's tooth. For durability, a hard durable casing, such as a dental composite resin, is also suitable for a compliance device mounted in or otherwise coupled to the orthodontic appliance. In other embodiments, the casing can comprise an elastomeric material (e.g., a silicone, a silicone rubber such as the Silbione® liquid silicone rubber, and/or the like). In other embodiments, the casing can be fabricated from a medical-grade injection-molded, highly flexible and very low durometer silicone. In some embodiments, the casing can be constructed from a material similar to or the same as, or having physical characteristics in common with a material of which the orthodontic appliance is constructed (e.g., a material of which a transparent, removable aligner is constructed). In some embodiments, the casing is constructed of a hermetic material. In this manner, the compliance device is configured to be water- or moisture-proof (or water- or moisture-resistant), thereby protecting the components of the compliance device from moisture-related damage that might otherwise occur as a result of being disposed in the patient's oral cavity. In some embodiments, the casing of the compliance device can have shape-memory characteristics. In some embodiments, at least a portion of the compliance device, such as at least a portion of the casing, comprises an optically transparent material (e.g., a window or a clear panel). The optically transparent material is configured to allow, in some embodiments, optical communication from the sensor of the compliance device at least partially disposed within the casing to the light therapy apparatus, and vice versa. In some embodiments, the optically transparent material can be translucent silicone. In some embodiments, the case can comprise of more than one material. For example, a portion of the casing can comprise a hard, durable material, and another portion of the casing (for example, that covering, encasing or surrounding a force sensor configured to be depressed by an orthodontic appliance) can comprise a more flexible material such as silicone or rubber).

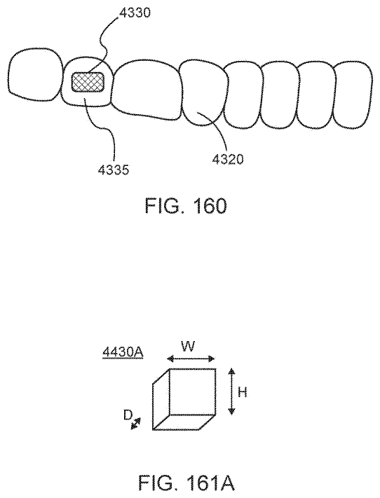

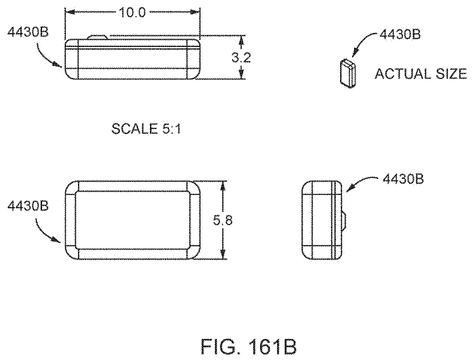

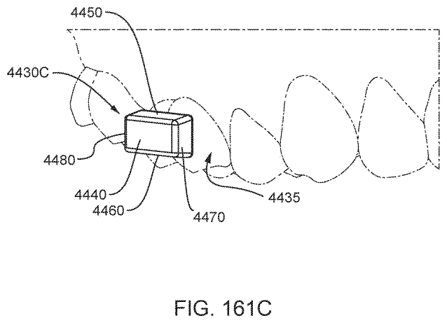

The compliance device, and in some embodiments, the casing of the compliance device, can have any suitable dimension for being disposed in the patient's mouth, coupled to a patient's tooth, and/or for being coupled to the orthodontic appliance. In some embodiments, the compliance device is of a suitable size to fit fully on the surface of a single tooth, as depicted in the schematic embodiment in FIG. 160 and the embodiment in FIG. 161C. In some embodiments, the compliance device can be significantly smaller than the surface of a tooth. For example, the compliance device can be similar in many respects, or identical to, including having a size and/or dimensions similar or identical to, compliance device described herein with respect to FIG. 161A, 161B, or 161C.

In some embodiments, the compliance device has a size, shape, dimension or other form factor that avoids or minimizes any potential deformation of the orthodontic appliance, e.g., as a result of coupling the compliance device thereto or as a result of disposing the orthodontic appliance to overlie and optionally contact the compliance device coupled to the tooth, or to avoids or minimize any difference in user experience with the orthodontic appliance in comparison to user experience with the orthodontic appliance in the absence of the compliance device. As illustrated in the schematic depiction in FIG. 161A, a compliance device 4430A in some embodiments can have a substantially cuboid form with an associated width W, height H, and depth D. In some embodiments, a compliance device can have a frustopyramidal form. In other embodiments, a compliance device can have a substantially cylindrical or spherical form.

In embodiments in which a compliance device has a cuboid form, the compliance device can have a substantially similar height and width, and can have a depth less than the height and/or width. In some embodiments, the width W can be from about 2 mm to about 12 mm, including all values and sub-ranges in between. For example, the width W can be about 4 mm. In some embodiments, the height H can be from about 2 mm to about 6 mm, including all values and sub-ranges in between. For example, the height H can be about 4 mm. In some embodiments, the depth D can be from about 2 mm to about 4 mm, including all values and sub-ranges in between. For example, the depth D can be about 3 mm. In some embodiments, the compliance device 4320 can have dimensions substantially equal to or less than about 4 mm wide, about 4 mm high and about 3 mm deep. As depicted in the illustrative embodiment in FIG. 161B, a compliance device 4430B can have a width of about 10 mm, a height of about 5.8 mm, and a depth of about 3.2 mm. As depicted in FIG. 161C, which is drawn to scale, a compliance device 4430C can have a width of about 8.5 mm, a height of about 4.9 mm, and a depth of about 2.9 mm. Although the compliance device 4430A is shown in FIG. 161A as having a substantially cuboid shape, in other embodiments, the compliance device can have any suitable shape and/or dimensions. In some embodiments, the compliance device can have a rectangular, square, circular or trapezoidal cross-sectional shape along a transverse axis of the device.

In some embodiments, the compliance monitoring device can have a form factor that readily allows an orthodontic appliance (e.g., an aligner) to be applied to and/or removed from a user's teeth while the compliance monitoring device is positioned within a user's mouth (e.g., bonded to a molar). As shown in FIG. 161C, the compliance monitoring device can have a first side 4440 (front side, outward facing when positioned in a user's mouth), a second side (back side, facing a user's tooth when positioned in a user's mouth, depicted in contact with premolar 4435) opposite and generally parallel to the first side, a third side 4450 (top side, facing upward when positioned in a user's mouth), a fourth side 4460 (bottom side, facing downward when positioned in user's mouth), a fifth side 4470 (right side, anterior facing when positioned in a user's mouth), and a sixth side 4480 (left side, posterior facing when positioned in a user's mouth). In some embodiments, one or more of the third, fourth, fifth, and sixth sides 4450, 4460, 4470, 4480 can be generally perpendicular to the first side 4440, while in other embodiments, one or more of the third, fourth, fifth, and sixth sides 4450, 4460, 4470, 4480 can be at an obtuse angle relative to the first side 4440. For example, in some embodiments, the angle formed between the first side 4440 and the third side 4450 and/or the fourth side 4460 can be between about 91 degree and about 120 degrees, including any values and sub-ranges in between, such that the compliance monitoring device can have a trapezoidal cross-sectional shape (taken along the device's transverse axis). In some embodiments, the angle formed between the first side 4440 and the third side 4450 and/or the fourth side 4460 can be about 95 degrees. Put another way, in some embodiments, one or more of the third, fourth, fifth, and sixth sides can be angled or canted. Additionally or alternatively, in some embodiments, the edges formed between the first side 4440 and any or all of the third, fourth, fifth, and sixth sides 4450, 4460, 4470, 4480 can be rounded, and/or any or of all of the corners of the compliance monitoring device can be rounded. In some embodiments, the second side can be curved (e.g., concave), which can assist is coupling the compliance monitoring device to the surface of a user's tooth.

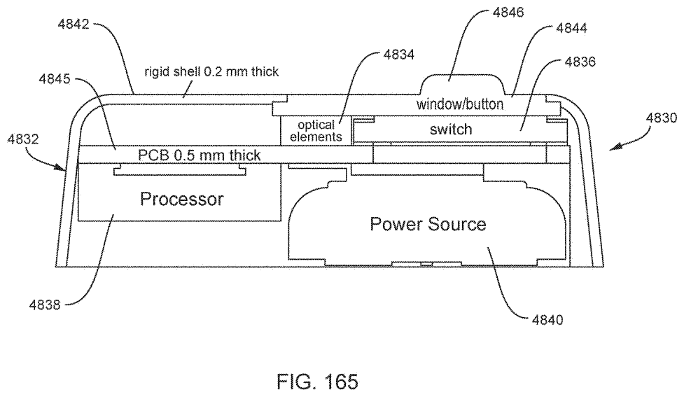

FIG. 165 depicts a cross-sectional view of an embodiment of a compliance monitoring device. As shown there, the compliance monitoring device 4830 can comprise a housing or casing 4832, a transceiver 4834, a sensor 4836, a processor 4838, a power supply 4840, and a printed circuit board 4845. The housing 4832 can comprise a rigid portion 4842 and an optical window 4844, and can comprise a frustropyramidal shape forming a cavity therein. The transceiver 4834, the sensor 4836, the processor 4838, the power supply 4840, and the printed circuit board 4842 can be disposed within the cavity. The rigid portion 4842 can be formed from any suitable rugged, biocompatible material as described herein, for example, stainless steel and/or plastic (e.g., polycarbonate plastic). The optical window 4844 can be formed in a top or outer surface (outward facing when positioned on a user's tooth) of the compliance monitoring device 4830, and can comprise a protrusion or raised portion 4846 that extends outward from an external surface of the optical window 4844 aligned with the external surface of the rigid portion 4842 (e.g., the raised portion 4846 has a greater thickness than the unraised portion of the optical window 4844). The optical window 4844 can comprise a translucent or transparent flexible material (e.g., silicone).

The housing 4832 can enclose and protect the transceiver 4834, the sensor 4836, the processor 4838, the power supply 4840, and the printed circuit board 4845. In some embodiments, one or more of the transceiver 4834, the sensor 4836, the processor 4838, the power supply 4840, and the printed circuit board 4845 can be positioned or encapsulated in an insulating material (e.g., an insulating resin), that can protect the one or more components and stabilize or otherwise fix the position(s) of the one or more components within the housing 4832. In some embodiments, all of these components (i.e., the transceiver 4834, the sensor 4836, the processor 4838, the power supply 4840, and the printed circuit board 4845) can be positioned or encapsulated in an insulating material. In some embodiments, the insulating material can form a bottom, or tooth-facing surface, of the compliance monitoring device 4830. In some embodiments, including those in which the insulating material forms the bottom surface of the compliance monitoring device 4830, the bottom surface can have a curved (e.g., concave) shape that is configured to conform to or mate with a curved (e.g., convex) surface of a user's tooth. In these embodiments, the bottom surface can be shaped using any suitable technique, for example, machining, grinding, and/or molding.

The transceiver 4834, the sensor 4836, the processor 4838, and the power supply 4840 can be any of the transceivers, sensors, processors, and power supplies described herein respectively. For example, in some embodiments, the transceiver 4834 can comprise optical elements (e.g., one or more LEDs and/or photodiodes), the sensor 4836 can comprise a pressure sensor (e.g., a pressure transducer or switch), the processor 4838 can comprise a microcontroller, and/or the power supply can comprise a non-rechargeable battery (e.g., a coin cell battery) or a rechargeable battery (e.g., a lithium-manganese battery). In some embodiments, the microcontroller can comprise a clock or timer. Additionally or alternatively, in embodiments in which the pressure sensor comprises a pressure transducer, the pressure transducer can comprise a TACT or dome switch that can be activated by external pressure applied to the surface of the switch. In other embodiments, the pressure transducer can comprise a pressure sensitive resistor or a resistor bridge array.

One or more of the transceiver 4834, the sensor 4836, the processor 4838, and the power supply 4840 can be positioned on, coupled to, or otherwise in communication with the printed circuit board 4845. The printed circuit board 4845 can be a rigid or a flexible printed circuit board comprising one or more conductive layers. Suitable attachment and interconnect technologies include solder processes and chip on board wirebonding. In some embodiments, flexible circuit or leadframe (e.g., stamped or etched leadframes) substrates and chip-and-wire or chip-on-board components can be used.

In the embodiment depicted in FIG. 165, the sensor 4836 comprises a TACT switch that is positioned adjacent to, and just below (i.e., closer to a user's tooth surface when coupled to a user's tooth), the optical window 4844, and the transceiver 4834 comprises optical elements (e.g., an LED and photodiode) also positioned adjacent to, and just below, the optical window 4844. Thus, the optical elements can be positioned aside the switch. The bottom or inner surface of the optical window 4844 can be coupled to or mate with the switch, such that, in use, the raised portion 4846 of the optical window 4844 can serve as a button that can be used to activate or deactivate the switch when the button is depressed by an orthodontic appliance (e.g., an aligner) that is placed in a user's mouth. Pressure applied to the raised portion 4846 of the optical window 4844 by an orthodontic appliance can be transferred to the switch, which can allow the compliance monitoring device 4830 to sense the presence or removal of the orthodontic appliance. The optical window 4844 can also be used to transmit light to and/or from the optical elements, thus allowing the compliance monitoring device 4830 to communicate with a probe, light therapy apparatus, or other external device.

Orthodontic Appliances

Systems can also comprise one or more orthodontic appliances. The orthodontic appliance(s) can be any suitable orthodontic appliance useful for moving one or more teeth of the patient. In some embodiments, the orthodontic appliance is removable. Examples of removable orthodontic appliances include, but are not limited to, Active Hawley appliances, transparent aligners (such as INVISALIGN™ aligners), aligners, fan expanders, or sagittal appliances. In some embodiments, the orthodontic appliance is a clear or transparent aligner, such as an INVISALIGN™ aligner. The compliance monitoring system, and the compliance device 4220A, 4220B, 4220C, 4225 specifically, is particularly useful with respect to removable orthodontic appliances, because the actual time the patient's uses or wears such a removable orthodontic appliance can vary based on individual circumstances for each patient, and such actual “wear time” (i.e., the time the patient uses or wears the orthodontic appliance) can be important in determining the effectiveness and/or structure of an orthodontic treatment program.

Although the compliance monitoring system is particularly useful with respect to removable appliances, in some embodiments, the orthodontic appliance is fixed. Such orthodontic appliances can be fixed for a predetermined period of time, e.g., by an orthodontist or other healthcare professional, to one or more of the patient's teeth during orthodontic treatment, as described herein. Examples of suitable fixed orthodontic appliances include, but are not limited to, pin and tube appliances, ribbon arch appliances, Begg Lightwire appliances, edgewise appliances, pre-adjusted edgewise appliances, self-ligating edgewise appliances, hi-helix appliances, tri-helix appliances, quad helix appliances, rapid maxillary expansion appliances (RME), or pin stripe appliances, or any other fixed orthodontic appliance described herein. In some embodiments, the patient can wear both a fixed orthodontic appliance and a removable orthodontic appliance.

Data Communication Devices

In some embodiments, systems can also comprise a data communication device. In some embodiments, the systems comprise a plurality of data communication devices. A data communication device can be or include any suitable data communication device, including a light therapy apparatus, probe, other external dental equipment, an external computational device or processor (including a mobile phone, mobile computer, external processor or cloud-based processor, or any other suitable computational device), or any other data communication device. Data communication devices can be configured to receive and/or transmit data from/to the compliance monitoring device. For example, the compliance monitoring device can be configured to communicate data received from the sensor to the data communication device (e.g., from memory, via a processor, or from the sensor directly). In another example, the data communication device can transmit data to the compliance monitoring device, such as operation instructions (e.g., sampling rate of the sensor, how data should be stored in memory, how often the compliance monitoring device should communicate with the communication device). Once data from the compliance monitoring device is received by a data communication device, the data communication device can send the data to another application or location, for example, to a software application on a mobile device or computer (e.g., an app), an electronic medical record or other user medical file, and/or a server (e.g., a cloud) for storage. The data can be uploaded on regular intervals, e.g., hourly, daily, and/or upon receipt of new data from the data communications device. In some embodiments, the data received can be used to generate real-time alerts to inform a user and/or medical providers or clinical staff (e.g., daily, weekly) of a user's compliance with a prescribed treatment program.

Probe

In some embodiments, systems comprise a probe configured to receive from and/or transmit data to the compliance monitoring device. For example, a probe can be configured to receive or transmit data when brought into close proximity with a compliance monitoring device. In some embodiments, when the probe is brought into close proximity (e.g., near a user's mouth), the compliance device automatically transmits data to the probe, or transmits data in response to a signal from the probe (e.g., a pulse of light). In some embodiments, the compliance device transmits data wirelessly (e.g., via optical signals). A probe can have any suitable dimension or configuration. In some embodiments, the probe is a small, handheld device such that it is easily placed in proximity to a compliance monitoring device. In some embodiments, the probe (e.g., the entire probe, only a portion of the probe, such as a portion comprising a photodiode, a portion comprising an LED, a portion comprising both a photodiode and an LED) is configured to be placed within a patient's mouth such that the probe can collect data from the compliance monitoring device while the compliance monitoring device is positioned within the patient's mouth.

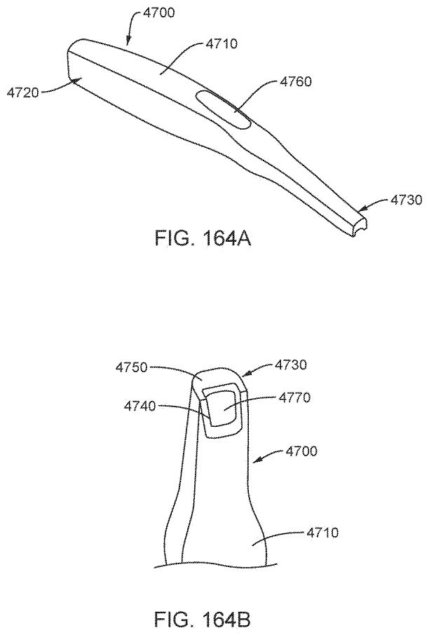

FIGS. 164A and 164B depict one embodiment of the probes described herein. FIG. 164A provides a top perspective view of the entire probe 4700, while FIG. 164B provides a bottom perspective view of a distal end of the probe 4700 depicted in FIG. 164A. The probe 4700 can comprise a housing 4710, a data collection control 4760, a processor, an LED and a photodiode. The processor, LED, and photodiode can be disposed within the housing 4710 and can be used to optically communicate with a compliance monitoring device, as described in more detail herein. The probe can further comprise a cable connection interface (e.g., a USB port), which can be used to provide power to the probe and/or to transfer data collected by the probe to an external device (e.g., a computer), via a cable (e.g., a USB cable). In some embodiments, the probe comprises a battery, e.g., a rechargeable or non-rechargeable battery, which can provide power to the electrical components (e.g., LED, photodiode, processor) of the probe 4700. Additionally or alternatively, the probe 4700 can be configured to wirelessly communicate (e.g., via Bluetooth or the like) with an external device to transfer collected data.

As shown in FIG. 164A, the housing 4710 can comprise a proximal end 4720, which is useful as a handle to hold and direct the probe 4700, and a distal end 4730, which is configured to fit within a user's mouth. The housing 4710 can further comprise an opening 4740 at the distal end 4730, which can provide for optical communication between the LED and the photodiode of the probe 4700 and a compliance monitoring device. In some embodiments, the opening 4740 can be positioned on a bottom surface or a top surface of the housing 4710 between the proximal end 4720 and the distal end 4730, and can intersect the surface forming the distal tip 4750. For example, as depicted in FIG. 164B, the opening 4740 can be formed in a bottom surface of the housing 4710 and can extend distally through the surface of the distal tip 4750 such that an open channel is formed (e.g., the bottom and distal end of channel can be open; the channel can be generally U-shaped along a longitudinal axis of the probe 4700 as depicted in FIG. 164B and the channel can be generally U-shaped along a transverse axis of the probe as is shown in FIG. 164A). This configuration can allow the opening 4740 to easily slide over and/or around a compliance monitoring device while it is coupled to a user's tooth. The opening 4740 can comprise a shape or size that accommodates, receives or corresponds to the shape or size of a compliance monitoring device, which can allow the probe to easily releasably couple to the compliance monitoring device. For example, the opening 4740 can comprise a generally open rectangular shape (e.g., U-shaped) with radiused corners such that it can receive a compliance monitoring device, such as that depicted in FIG. 161C.

The housing 4710 can further comprise an optically transparent material coupled to and positioned within the opening 4740 (e.g., forming a top surface of the channel), which can allow for optical communication between the electronic components (e.g., the LED, photodiode) positioned within the housing 4710 and a compliance monitoring device, while protecting the electronic components from, for example, moisture and contamination. That is, the optically transparent material can form an optical window 4770 in the housing 4710. In other embodiments, the opening 4740 can be formed entirely on the surface of the distal tip 4750. In some embodiments, the optically transparent material is not present, and the opening 4740 serves as the optical window.

In some embodiments, the housing 4710 is formed from two rigid, plastic housing portions (e.g., a top portion and a bottom portion) that can be attached to one another (e.g., using adhesive, fasteners, a snap fit configuration, or the like) to enclose and/or protect the processor, the LED, and the photodiode. In other embodiments, the housing 4710 is formed from a single housing portion, or from more than two housing portions (e.g., three, four, or more).

As described herein, the probe 4700 can comprise an LED, a photodiode, and a processor disposed within the housing 4710. The LED and the photodiode can be positioned at the distal end 4730 of the probe 4700 adjacent the optical window such that the LED and photodiode align with the LED and photodiode of the compliance monitoring device when the probe is positioned in proximity to (e.g., releasably coupled with) the compliance monitoring device. The LED and photodiode can be communicatively coupled to the processor. The processor can be positioned at any suitable location within the housing 4710 and can be, for example, a general-purpose processor, a microprocessor, a microcontroller, a field programmable gate array (FPGA), an application specific integrated circuit (ASIC), and/or the like. In some embodiments, the processor can comprise memory, for example, Read-Only Memory (ROM), Random-Access Memory (RAM), electrically erasable programmable read-only memory (EEPROM), and/or any suitable non-transitory processor-readable medium. In other embodiments, the memory can be, or can be included in, a separate component electrically coupled with the processor.

As depicted in FIG. 164A, the housing 4710 can comprise a data collection control 4760. The data collection control 4760 can be moveably coupled to the housing 4710 and can be configured to allow a user to control data collection from a compliance monitoring device. For example, the data collection control 4760 can be or comprise a button, a knob, a slider, a switch, or the like, that can be communicatively coupled to the LED, photodiode, and/or the processor. The data collection control 4760 can be positioned at location that is easily accessible by a user holding the housing 4710, for example, on a top, bottom, or side surface of the housing 4710. Actuation of the data collection control 4760 (e.g., in embodiments in which the data collection control 4760 is a button, depression of the button), can begin a data transfer sequence between the probe 4700 and a compliance monitoring device. In some variations, the probe 4700 further comprises an audible, visual, and/or tactile feedback generator (e.g., a beeper, a light source (e.g., LED), a vibrator, or the like), which can be coupled to the data collection control 4760 and can provide an audible, visual, and/or tactile indication that the data collection control 4760 has been actuated. In one embodiment, the probe can comprise an audible feedback generator (e.g., a beeper) and/or a visual feedback generator (e.g., an LED); however, the probe can comprise any combination of audible, visual, and tactile feedback generators.

In use, the probe 4700 can collect data from a compliance monitoring device via optical communication. For example, the distal end 4730 of the probe 4700 can be placed in close proximity to (including in direct contact with) the compliance monitoring device while the compliance monitoring device is within a user's mouth and/or after removal of the compliance device from the user's mouth. The distal end 4730 of the probe 4700 can be advanced over the compliance monitoring device such that the compliance monitoring device is received, partially or fully, within the opening 4740. A user can actuate the data collection control 4760, which can cause the probe 4700 to transmit a light pulse through the optical window 4770 of the probe 4700 to and through an optical window in the compliance monitoring device. This light pulse can “wake” the compliance monitoring device and activate the data communication mode. The probe 4700 can transmit a light pulse sequence to the compliance monitoring device and the compliance monitoring device can verify the sequence is genuine. In some embodiments, the probe 4700 can transmit a single light pulse sequence to “wake” the compliance monitoring device, activate the communication mode, and identify/verify the probe 4700, while in other embodiments, the probe 4700 can transmit different light pulses or light pulse sequences for one or more of “waking” the compliance monitoring device, activating the data communication mode, and identifying/verifying the probe 4700. The compliance monitoring device can then send data obtained by its sensor to the probe using pulsed light, as described in more detail herein. The pulsed light can be received by the probe through the optical window 4770 and the probe 4700 can optionally send a light pulse (or light pulse sequence) to the compliance monitoring device to confirm receipt of the data. The distal end 4730 of the probe 4700 can then be removed from the user's mouth and the compliance monitoring device can exit the data communication mode. After the data is transferred to the probe, it can be erased from the compliance monitoring device. The probe can store and/or transmit the collected data to another device (e.g., a computer) using a wired (e.g., USB cable) or wireless (e.g., Bluetooth) connection. The data transfer described herein can be completed quickly, for example, in less than about 5 seconds, less than about 2 seconds, between about 1 second and about 5 seconds, or between about 2 seconds and about 5 seconds.

Light Therapy Apparatus

In some embodiments, systems for compliance monitoring can comprise a light therapy apparatus and the light therapy apparatus can operate as a data communication device. The light therapy apparatus can be or comprise any light therapy apparatus disclosed herein. For example, the light therapy apparatus can be or comprise a light therapy apparatus described herein that comprises a mouthpiece configured to be disposed in a patient's mouth, including, for example, apparatus 2100, 2500, 3000, 3100, 3300, 3600, 3700, 3800, and mouthpiece 3910. As described herein, the light therapy apparatus can be configured to electrically communicate with (e.g., at least one of send or receive an electronic communication, such as a wireless signal, to or from, respectively) the compliance device 4220A. 4220B, 4220C, 4225. For example, the light therapy apparatus can be configured to receive raw data from the sensor related to use of an orthodontic appliance. In some embodiments, the light therapy apparatus can process the data from the sensor to determine user compliance with the use of an orthodontic appliance. The light therapy apparatus can be additionally or alternatively configured to recharge the power source of the compliance monitoring device. Such as, for example, by transmitting light to the compliance monitoring device.

In some embodiments, the light therapy apparatus is configured to wirelessly receive/transmit data to and from the compliance monitoring device using, for example, an additional one or more (e.g., 2, 3, 4, or more) LEDs (data communication LEDs) and an additional one or more (e.g., 2, 3, 4, or more) photodiodes (data communication photodiodes). The data communication LED(s) and photodiode(s) can be positioned in or on the light therapy apparatus such that they align with the compliance monitoring device (e.g., with an LED and photodiode in the compliance monitoring device) when the light therapy apparatus is positioned in the user's mouth. For example, in an embodiment in which the light therapy apparatus is or comprises mouthpiece 3910, the data communication LED(s) and photodiode(s) can be positioned below the array of light emitters 3944 (e.g., between the bottom row of light emitters 3944 and the bite tray 3912). Once the data communication LED(s) and photodiode(s) are aligned with the compliance monitoring device, data can be transferred from the compliance monitoring device to the light therapy apparatus using the same or a similar technique as described herein with respect to the probe. In some embodiments, simply positioning the light therapy apparatus in the user's mouth can “wake” the compliance monitoring device, while in other embodiments, the light therapy apparatus can “wake” the compliance device using one or more light pulses.

System Operations

As described herein, systems described herein can be used to measure user compliance, such as compliance with use of an orthodontic appliance. Thus, compliance devices can be situated in the mouth of a user (e.g., coupled to the tooth of a user and/or coupled to an orthodontic appliance) to track the use of orthodontic appliances. Although the compliance monitoring devices and systems are primarily described as measuring compliance with use of one or more orthodontic aligners, in some embodiments, the compliance monitoring devices and systems can measure compliance with the use of a light therapy apparatus. The compliance device can be bonded to the tooth of a patient, and can be configured to detect the presence and/or use of orthodontic appliances such as aligners and/or light therapy apparatus. In another example, the compliance device can be bonded to an orthodontic appliance. The compliance device can be configured to detect an input indicating the presence of an orthodontic aligner and/or the presence of a light therapy apparats.

In some embodiments, the compliance device can have various modes of operation, including an sensing mode, a charging mode, and a communication mode. In some embodiments, the system (and/or the compliance device) is operable in two or more modes concurrently (or, during at least overlapping time periods). For example, the system (and/or the compliance device specifically) can concurrently, or in an overlapping time period, operate in the sensing mode and the charging mode, in the charging mode and the communication mode, in the sensing mode and the communication mode, or in all three modes. In some embodiments, operation of the system (and/or the compliance device) in one or more of the operating modes can be mutually exclusive of operation of another of the operating modes. For example, in some embodiments, when the system (and/or the compliance device specifically) is operating in the communication mode, the system (or compliance device specifically) can be prevented (e.g., via circuitry, such as via the opening or closing of one or more switches) from concurrently operating in a different operating mode (e.g., the sensing mode, the charging mode, or both). In another example, in some embodiments, when the system (and/or the compliance device specifically) is operating in the sensing mode, the system (or compliance device) can be prevented from concurrently operating in a different operating mode (e.g., the charging mode, the communicating mode, or both). In still another example, in some embodiments, when the system (and/or the compliance device specifically) is operating in the charging mode, the system (or compliance device) can be prevented from concurrently operating in a different operating mode (e.g., the sensing mode, the communicating mode, or both).

Sensing Mode

The compliance monitoring device can be configured to track compliance in any suitable manner, for, example, based on sensing a particular input. Compliance monitoring devices described herein can be configured to detect various inputs. Compliance monitoring devices can be configured to detect inputs in any suitable manner. In some embodiments, the compliance monitoring device can be configured to detect an input (e.g., a physiological parameter, force, pressure, etc.) at predetermined time intervals. For example, the compliance monitoring device can be configured to detect the physiological parameter at a first time, at a second time that is a predetermined duration subsequent the first time, and, optionally, at a third, fourth, or nth time (where n is any suitable positive integer, for example, from five to eighty) that is a predetermined duration subsequent the immediately preceding instance of physiological parameter detection by the compliance device. For example, in some embodiments, the sensor is configured to periodically attempt detection of the input, for example, at a predetermined time interval of about 1 minute, about 5 minutes, about 10 minutes, about 15 minutes, about 20 minutes, about 30 minutes, about 40 minutes, about 50 minutes, or about 60 minutes. Each instance of attempted input detection by the compliance device can occur for a predetermined duration, including a short or very short duration that helps to preserve battery life for the compliance device. For example, in some embodiments, the predetermined duration is within the range of about one hundredth of a second (0.01 second) to about nine hundredths of a second (0.09 seconds) or about one tenth of a second (0.1 second) to about nine tenths of a second (0.9 seconds), which helps to preserve battery life of the compliance device. In another example, the predetermined duration is about 1 second, about 2 seconds, about 5 seconds, about 10 seconds, about 15 seconds, about 20 seconds, about 30 seconds, about 45 seconds, about 1 minute, or any suitable duration within the range of about 1 second to about 1 minute or subranges therein. The passage of the time interval or the (attempted) detection duration can be determined based on the clock. In some illustrative embodiments, the compliance monitoring device can comprise circuitry that determines the presence of an input (e.g., temperature, force pressure, etc.). For example, where the input is force or pressure, the compliance device can periodically determine if the force sensor is on or off (e.g., if the force sensor is depressed by an aligner or not). In another example, where the input the temperature, the compliance device can periodically determine if the temperature is within a pre-determined temperature range by taking one or more temperature measurements.

In some embodiments, such as when the sensor of the compliance device is a force or pressure sensor, the compliance device can measure compliance based on how long a sensor is activated by an input or is inactive. The compliance monitoring device can be configured to track the amount of time a particular input in received, or the amount of time a particular input is not received. That is, the compliance monitoring device can provide compliance information based on the amount of time a device is in use (e.g., in a user's mouth), or the amount of time the device is not in use (e.g., not in a user's mouth). In some embodiments, the sensor comprises a component that, when placed under pressure by an orthodontic appliance, is moved (e.g., pushed downward) and either activates or deactivates a timing component. In some of these embodiments, the compliance device is not configured to sense an input at predetermined time intervals, but is instead configured to trigger some type of action (e.g., turning a component such as a timer on or off) upon receipt of an input. For example, when the pressure or force sensor is depressed a timer can be stopped (or started) and the compliance monitor can therefore determine compliance based on how long the aligner is in or out of the user's mouth.

In some embodiments, the sensor comprises a switch (e.g., a micro-switch) that is positioned and configured to be depressed by an orthodontic appliance when the orthodontic appliance is in use (e.g., in a user's mouth). In some embodiments, the switch is positioned on or otherwise coupled to an external surface of the casing of the compliance device, or is positioned adjacent to an external surface of the casing. For example, the switch can be positioned adjacent to, coupled to, or otherwise form part of an optical window in the casing of the compliance device. The switch can be coupled to a circuit that can remain open when the switch is not depressed and can close when the switch is depressed, or vice versa. The circuit can be coupled to or can otherwise communicate with a timer that can track or monitor whether the circuit is open and/or closed, thus providing an indication as to whether the orthodontic appliance is and/or is not in use. The timer can continuously or intermittently (e.g., using intervals of, for example, every 5 minutes, every 10 minutes, every 30 minutes, every hour, every 2 hours) determine whether the circuit is open or closed. In some embodiments, the timer can be triggered by a change in the position of the switch, after which it can intermittently or continuously monitor whether the circuit is open or closed. The timer can determine the amount of time the circuit is open, the amount of time the circuit is closed, or both, thereby providing the amount of time the orthodontic appliance is and/or is not in use. In some embodiments in which the timer intermittently monitors whether the circuit is open or closed, the timer determines the amount of time by counting the number of time intervals the circuit is open and/or closed (i.e., the switch is depressed and/or not depressed). In one embodiment in which the sensor comprises a switch, the switch is coupled to a circuit that is closed when the switch is depressed and open when the switch is not depressed. The circuit can be coupled to a timer that can be activated when the circuit is open and the switch is not depressed, and can intermittently determine, for example, in time intervals of 30 minutes to 1 hour, whether the circuit is still open. In some embodiments, the timer is inactive when the switch is depressed. In this embodiment, the switch can be depressed when an orthodontic appliance (e.g., an aligner) is in use (e.g., fully seated on a user's teeth) and not depressed when the orthodontic appliance is not in use. The timer can track the number of intervals, and thus the amount of time, the switch is not depressed, thus allowing the compliance device (or an external device to which the data is transferred, e.g., a data communications device such as a probe or light therapy apparatus, or computer) to determine to the amount of time the orthodontic appliance is not in use. This time data (e.g., a bit sequence representing the number of intervals counted) can be stored on the compliance device and transmitted to a probe or other external device as described herein. While described in this embodiment as comprising a switch that opens a circuit when an orthodontic device is not in use, thereby tracking the amount of time the orthodontic device is not in use, the compliance device can alternatively comprise a switch that opens a circuit when an orthodontic device is in use, thereby tracking the amount of time the orthodontic device is in use, as also described herein.

Charging Mode

As described herein, compliance monitoring devices can operate in a charging mode. For example, compliance devices can have rechargeable power sources, such as batteries, which can be recharged in any suitable manner. In some embodiments, the power sources can be rechargeable using a light therapy apparatus, such as those described herein. However, a power source can be recharged in any suitable manner, and by any suitable device. It should be understood that, although the examples below teach using light emitted from a light therapy apparatus to charge a power source of a compliance monitoring device, the compliance monitoring device can be charged by light delivered from any suitable device.

The charging mode of the compliance device can be activated by the light therapy apparatus. The light therapy apparatus can activate the compliance device by switching the power switch of a circuit in the power source of the compliance monitoring device to an “on” position. For example, the charging mode of the compliance device can be activated once the light therapy apparatus begins emitting light (e.g., the compliance device is activated upon receipt of light form the light therapy apparatus). In some embodiments, the light therapy apparatus can begin to emit light once it is properly positioned in a user's mouth. In other embodiments, the light therapy apparatus can begin to emit light once it is removed from a docking or charging station. The light therapy device can begin to emit light automatically (e.g., when properly positioned, or when removed from a docking/charging station), or it can require some form of user input to begin emitting light. A user input to activate light emission from the light therapy apparatus can be any suitable input, such as pushing a button, flipping a switch, applying force to the apparatus once it is positioned in the user's mouth (e.g., pushing the apparatus into place or biting down on the apparatus), or remotely activing the apparatus (e.g., using an application on a mobile device or computer). In some embodiments, light at a particular wavelength or range of wavelengths will activate the charging mode of the compliance device, but light at other wavelengths will not. In some embodiments, the compliance device can exit the charging mode automatically, for example, upon receipt of instruction from the light therapy apparatus, or by sensing that light is no longer being delivered. In other embodiments, the compliance device can respond to user input to exit a charging mode.

In some embodiments, a power source is charged using light from the a light therapy apparatus. In some of these embodiments, the power source is a rechargeable battery comprising a circuit configured to charge the compliance monitor upon receipt of light. The circuit can comprise a power switch, which, when in a first position (e.g., open or closed), can prevent the device from being charged. When the power switch is in a second position (e.g., closed or open), the device can be charged. As described herein, the power switch can be light-activated. For example, in some embodiments, the compliance device comprises an optically transparent window with a light detector (e.g., a photosensor, an LED, or the like) positioned behind the window. Light can be transmitted through the optical window to the light detector, using, for example, an external transceiver. When the light detector receives a sufficient intensity of light, the position of the power switch can change (e.g., from the first position to the second position or vice versa), thus preparing the power source for charging. The circuit can be configured to be charged by light in any suitable manner.

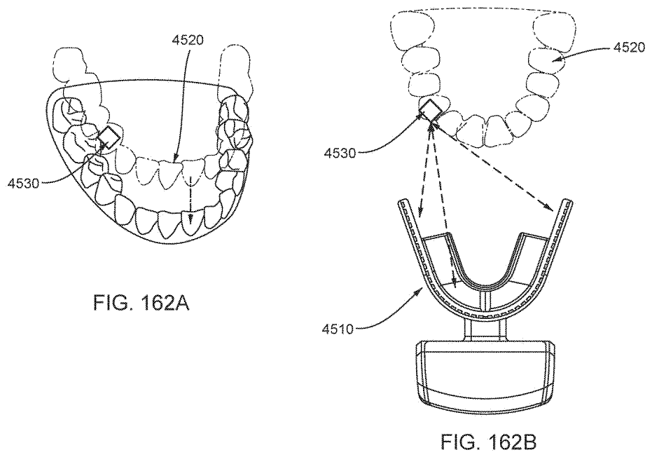

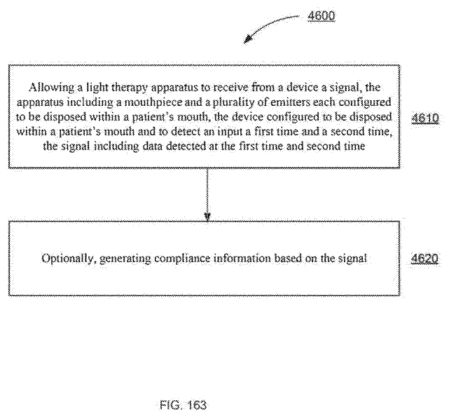

FIG. 162B depicts an illustrative embodiment of a compliance device 4530 disposed in the mouth of a user and the light therapy apparatus 4510 collectively configured to charge the compliance device 4530 by delivery or transmission of light from the light therapy apparatus 4510 to the compliance device 4530. Although FIGS. 162A and 162B depict a compliance device and an aligner in the mouth of a user, it should be understood that the system describe below to charge the compliance monitoring device is operable without an aligner in place, and the compliance monitoring device can be attached to the tooth of a user or to an aligner. The light received by the compliance device 4530, such as by a light transceiver such as light transceiver, from the light therapy apparatus 4510 can be used to charge the compliance device 4530 (e.g., to charge a power source such as power source 4238), as described herein with respect to compliance device 4220A, 4220B, 4220C, 4225. The compliance device can be configured to be charged once (e.g., upon a first use by the patient of the compliance device 4520), periodically (e.g., once a day, every other day), or on demand (e.g., upon user input provided to the light therapy apparatus). In some embodiments, the compliance device 4530 is configured to automatically enter the charging mode in response to receiving the transmission of light from the light therapy apparatus 4510. In some embodiments, the compliance device 4530 is configured to optically receive from the light therapy apparatus 4510 an instruction to enter the charging mode (e.g., prior to or substantially simultaneous with receiving light intended for charging the device). Similarly, in some embodiments, the compliance device 4530 is configured to exit the charging mode based on receipt of an instruction from the light therapy apparatus 4510. In some embodiments, the compliance device 4530 can be configured to automatically exit the charging mode when the transmission of light (for charging) ceases. In some embodiments, the compliance device 4530 is configured to exit the charging mode when the power source has reached a threshold charge. In this manner, the compliance device 4530 is configured to prevent overcharging of the power source. In some embodiments, the compliance device 4530 is configured to be in the charging mode charged during substantially an entirety of a treatment session. In some embodiments, the compliance device 4530 is configured to be in the charging mode during part of (i.e., less than an entirety of) a treatment session.

In some embodiments, any suitable light delivery wavelength and format can be employed by the light therapy apparatus for charging the compliance device. In some embodiments, the compliance device is configured to charge the power source based on receipt of light emitted from the light therapy apparatus in a manner similar to the emission of light for tooth movement, as described herein. For example, in some embodiments, the light emitted by the light therapy apparatus and received by the compliance device for charging the power source during the charging mode can have a wavelength of about 850 nm (±5 nm). In some embodiments, the compliance device is configured to charge the power source based on receipt of light emitted from the light therapy apparatus at a wavelength different from a wavelength of light emitted by the light therapy apparatus for tooth movement, as described herein. In some embodiments, a light therapy apparatus comprises multiple zones of light emitters, of which at least a first zone of light emitters is configured to emit light having a first set of light characteristics (e.g., wavelength and format) and at least a second zone of light emitters is configured to emit light having a second set of light characteristics, different from the first set of light characteristics, such that the light having at least one of the first or second set of light characteristics is suitable for charging the compliance device and at least one of the first or second set of light characteristics is suitable for tooth movement, as described herein. In some embodiments, the light emitted by the light therapy apparatus during charging mode can be a continuous wave. In some embodiments, the light emitted by the light therapy apparatus during the charging mode can be pulsed. In some embodiments, the light therapy apparatus is configured to emit pulsed light for tooth movement and is configured to emit light in a continuous wave for charging the compliance device. In some embodiments, the light therapy apparatus is configured to emit light during charging mode for a duration of from about 2 minutes to about 6 minutes, including all values and sub-ranges in between.

In some embodiments, the compliance device is configured to charge the power source based on receipt of a light emitted by the light therapy apparatus for effecting an increased rate of tooth movement, as described herein. In other words, when, in use, the light therapy apparatus administers light to the patient to increase the rate of tooth movement, the compliance device is configured to use light received during the administering to charge the power source. For example, in some embodiments, the compliance device and the light therapy apparatus are collectively configured to simultaneously charge the compliance device and to orally administer light to facilitate tooth movement. In such a “treatment mode”, the light emitted by the light therapy apparatus mode can have a wavelength of about 850 nm (+5 nm). The compliance device can be configured, e.g., pre-configured, to be charged in this manner, and/or be configured to optically receive an instruction from the light therapy apparatus providing an indication of the treatment mode prior to receiving (or converting energy from) light intended for charging the compliance device and for treating the user. In some embodiments, the light therapy apparatus is configured to emit light during the treatment mode according to embodiments described herein. For example, in some embodiments, the light therapy apparatus is configured to emit light during the treatment mode for a duration of from about 2 minutes to about 20 minutes, including all values and sub-ranges in between.

Compliance Monitoring Device Placement

Compliance monitoring devices can be situated to monitor user compliance with an orthodontic appliance (or other suitable device) in any suitable manner. For example, compliance devices can be situated in the mouth of a patient, and can be coupled to a portion of the patient's mouth, an orthodontic appliance, a light therapy apparatus, or some combination thereof.

In some embodiments, the compliance device is configured to be coupled to the tooth of a patient. The compliance device can be coupled to any suitable portion of the tooth of a patient, for example, a buccal, lingual, or inferior surface of a tooth. The compliance device can be couplable to the tooth in any suitable manner. For example, the compliance device can be adhered to the tooth via an adhesive. Suitable adhesive materials include, but are not limited to, any suitable dental composite or dental resin, including those based on bisphenol A-glycidyl methacrylate (bis-GMA) resins. In some embodiments, the casing of the compliance device comprises an adhesive layer or material to allow removable attachment of the compliance device to the tooth of a user. In some embodiments, the tooth and/or the compliance device can comprise features to facilitate the bonding of the compliance device to the tooth, such as etching, grooves, holes, recesses, etc.

Systems can further comprise an attachment template configured to facilitate the placement of a compliance monitoring device on the tooth of a patient. In some embodiments, attachment templates can be an aligner-shaped device (e.g., a mold that conforms to the shape of a user's mouth and teeth) with one or more protrusions and can be customized to a user's teeth (i.e., can be patient-specific). The attachment template can be prefabricated from, for example, 3D printed plastics. The one or more protrusions can be configured to house a compliance device. Thus, when the attachment template (with the compliance monitoring device placed in the protrustion) is placed in the user's mouth, the compliance device can be placed at a pre-determined location on the tooth of a patient. The protrusion can hold the compliance device within the attachment template (e.g., so that the compliance device remains within the protrusion until the compliance monitoring device is properly situated to be adhered to a tooth) by any suitable means. For example, the compliance device can be friction fit to the inner surface of the protrusion, the compliance device can be magnetically attached to the inner surface of the protrusion, the compliance device can be removably adhered to the inner surface of the protrusion (e.g., with a removable epoxy), etc.

In order to attach the compliance device to the tooth of a user, the compliance device can be coated with a bonding agent. For example, the compliance device can be situated within the attachment template, coated with a bonding agent, and the attachment template with the compliance monitoring device can be placed into the mouth of the user such that the bonding agent adheres the compliance monitoring device to the tooth of the user. In some embodiments, the protrusion of the attachment template can be slightly deeper than the depth of the compliance device, such that there is room for a layer of bonding agent to be placed on the tooth-side of the compliance monitoring device (i.e. the side of the compliance device that will be attached to the patient's tooth) within the protrusion. In some embodiments, the attachment template can be relatively rigid. In other embodiments, the attachment template can be flexible, or can have a portion that is flexible. For example, the protrusion of the attachment template (and any additional portions) can be flexible, such as to allow a user to apply pressure to the protrusion in order to aid in the attachment of the compliance monitoring device to the tooth of the patient and/or the removal of the compliance monitoring device from the attachment template. In some embodiments, the user can bend or invert a flexible portion of the attachment template (e.g., the protrusion, a portion of the attachment template, the entire attachment template, or any combination thereof) in order to place the compliance monitoring device on the tooth of a user and/or remove the attachment template.

In some embodiments, the compliance monitoring device is configured to be coupled to the orthodontic appliance. The compliance device can be couplable to the orthodontic appliance in any suitable manner. In some embodiments, for example, the compliance device is embedded in the orthodontic appliance. In some embodiments, the compliance device is couplable to the orthodontic appliance via, for example, an adhesive, a fastener (e.g., snap-fit, hook-and-loop, or the like), a mating engagement, a resistance fit, bonding, molding, or the like, or any combination thereof. In some embodiments, for example, the casing of the compliance device can comprise an adhesive layer or material to allow permanent attachment of the compliance device to the orthodontic appliance. Suitable adhesive materials include, but are not limited to, any suitable dental composite or dental rein, including those based on bisphenol A-glycidyl methacrylate (bis-GMA) resins. In some embodiments, the casing of the compliance device comprises an adhesive layer or material to allow removable attachment of the compliance device to the orthodontic appliance. In some embodiments, the compliance device can be received in a pocket, recess, opening or the like of the orthodontic appliance. In some embodiments, the compliance device is removably couplable to the orthodontic appliance, for example, via one or more of the foregoing coupling mechanisms. In some embodiments, as described herein, the compliance device is directly coupled, permanently or removably, to a tooth or other oral tissue (e.g., via bonding to a tooth surface, such as, but not limited to, a buccal-facing or lingual-facing surface of the tooth).

FIG. 160 schematically illustrates an illustrative placement of a compliance device 4330 overlying a premolar 4335 of a set of teeth 4320. FIG. 161C depicts an illustrative placement of a compliance device 4430C overlying a premolar 4435, drawn to scale. Although the compliance device 4330, 4430C is shown in FIGS. 160 and 161C as being placed at a position proximate to or configured to overlay a premolar, in other embodiments, the compliance device can be coupled to the orthodontic device and/or tooth of a user in a different position or location (e.g., a location configured to be proximate to, overlying, coupled to, or otherwise associated with an incisor, a canine, a premolar, a molar, a tooth root, oral mucosa, alveolar mucosa, or the like). In the embodiments shown in FIGS. 160 and 161C, the compliance device 4330, 4430C is coupled to the premolar 4335, 4435, however, in some embodiments, the compliance device 4330, 4430C can be coupled to an orthodontic appliance, such as, an aligner. In some embodiments, the compliance device 4330, 4430C is embedded or recessed in a material or surface of the aligner. In some embodiments, the compliance device 4330, 4430B is a patch or distinct component that can be applied or coupled to any suitable surface of an orthodontic appliance or tooth of a user configured to receive light from a light therapy apparatus (not shown in FIGS. 160, 161C) during use. The surface can include, for example, an outer (e.g., buccal) surface of the orthodontic appliance or tooth, or an inner (e.g., lingual or tooth-facing) surface of the orthodontic appliance or tooth (e.g., if the material of the orthodontic appliance is sufficiently transparent, translucent or otherwise permits passage of light therethrough).

As described herein, the compliance device can be sized and shaped for placement within a user's mouth, such as permanently or removeably bonded to a user's tooth (e.g., a premolar). For example, in some embodiments, the compliance monitoring device can have a width ranging from about 3 mm to about 13 mm, a height ranging from about 2 mm to about 8 mm, and/or a depth ranging from about 1 mm to about 5 mm, inclusive of all values and sub-ranges therebetween in each range. In some embodiments, the compliance monitoring device can have width, height, and depth dimensions of about 8 mm, about 5 mm, and about 3 mm, respectively. In another embodiment, for example, the embodiment depicted in FIG. 161B, the compliance monitoring device 4430B can have width, height, and depth dimensions of 10 mm, 5.8 mm, and 3.2 mm respectively. In yet another embodiment, the compliance monitoring device can have width, height, and depth dimensions of 5.8 mm, 10 mm and 3.2 mm, respectively. FIG. 161C depicts an embodiment of a compliance monitoring device 4430C coupled to a premolar 4435, drawn to scale. In the embodiment depicted in FIG. 161C, the compliance monitoring device 4430C has width, height, and depth dimensions of 8.5 mm, 4.9 mm, and 2.9 mm, respectively.

In some embodiments, one or both of the perimeter and area of the compliance device is substantially the same as that of the tooth to which the compliance device is permanently or removably coupled. In some embodiments, the compliance device is substantially transparent or has a color that is substantially the same as that of the tooth to which the compliance device is permanently or removably coupled.

FIG. 162A illustrates placement of the orthodontic appliance 4520 (illustrated in FIG. 162A as an aligner) with a compliance device 4530 coupled thereto onto teeth of a user. FIG. 162B illustrates optical communication (see, e.g., the arrows in FIG. 162B) between the light therapy apparatus 4510 and the compliance device 4530. For illustration purposes only, light therapy apparatus 4510 is shown spaced apart from orthodontic appliance 4520. In use, however, the light therapy apparatus 4510 can be physically attached to, in contact with, immediately adjacent, overlying, or in close proximity to the orthodontic appliance 4520. The light therapy apparatus 4510 can be substantially similar to the apparatus 3800 illustrated in FIGS. 156A-156D, though it is understood that any of the apparatuses 2100, 2500, 3000, 3100, 3300, 3600, 3700, 3800,4210 and mouthpiece 3910 disclosed herein can be suitable for configuration as disclosed herein.

Data Communication

In some embodiments of systems described herein, various components of the system will be configured to communicate with other components. For example, components of the compliance monitoring device (such as the processor, memory, sensor, transceiver) can be configured to communicate with each other. Further, the compliance monitoring device can be configured to communication with other system components, such as data communication device. For example, the compliance monitoring device can be configured to transmit data received by the sensor to a data communication device, such as a probe, a light therapy apparatus, an external computational device, or any other suitable data communication device. In some embodiments, the data communication device can process the raw data received from the compliance monitoring device to determine a patient's compliance with orthodontic appliance usage during a treatment program. In other embodiments, a data communication device can be configured to send a signal to an external device (e.g., wirelessly, such as via Bluetooth® or via a wired connection, such as via a USB drive), such that the signal includes the detected input data (or portion thereof, including, for example “raw” or substantially unprocessed data) and its associated time/date data received from the compliance device. The external device can be configured to determine, based at least in part on the signal received from the data communication device, a patient's compliance with orthodontic appliance usage during a treatment program, as described in more detail herein.

In some embodiments, the compliance device is configured to wirelessly communicate (e.g., optically communicate via one or more LEDs) with a data communication device such as the light therapy apparatus or probe. The compliance device can communicate data associated with one or more stored inputs such as physiological parameters (e.g., stored temperatures), force/pressure, and time and/or date data associated with when each parameter was detected (or stored), such as in the communication mode. In the communication mode, in some embodiments, for example, the compliance device is configured to optically transmit a signal of data associated with inputs and the time and/or date when such inputs were detected by the sensor. The signal can be transmitted via light transceiver of the compliance device (e.g., by transmitting visible or infrared light). The data communication device (e.g. light therapy apparatus, probe, etc.) can be configured to optically receive (e.g., via light emitters 3844) the signal from the compliance device.

Although the compliance device is described herein as being configured for optical communication with data communication devices, in other embodiments, the compliance device can be configured to wirelessly communicate with data communication devices in a different manner, for example, via radio-frequency identification (RFID), near-field communication (NFC), or other suitable wireless communication.

In some embodiments, compliance with usage of one or more orthodontic appliances pursuant to an orthodontic treatment program is determined or otherwise calculated based, at least in part, on the input received by the compliance monitoring device. In some embodiments, temperature data can determine compliance with an orthodontic treatment program. For example, the temperature of the user's oral cavity will usually be at about normal body temperature (about 98.6° F., or about 37° C., ±0.5%). As such, a detected temperature within a predetermined temperature range inclusive of 37° C. can indicate that the orthodontic appliance to which the compliance device is coupled was disposed within the patient's mouth at the time of detection. Similarly, a set of detected temperatures within the predetermined temperature range can indicate that the orthodontic appliance to which the compliance device is coupled was disposed within the patient's mouth over a period of time beginning at least at a first time when a first temperature in the set of detected temperatures was detected and ending no earlier than a second time when a second (or most recently detected temperature in a set of detected temperatures was detected. If the duration from the first time to the second time is equal to or exceeds a prescribed duration during which the patient was to wear or use the orthodontic appliance, a determination can be made that the patient complied with the orthodontic treatment program. Described another way, data that are indicative of the patient's temperature and within the predetermined temperature range at each data point collected for a duration of time could indicate that the user was wearing the orthodontic appliance for that duration of time. In another embodiment, force/pressure data can be used to determine compliance with an orthodontic treatment program. For example, a sensor of a compliance monitoring device can be configured to record when a switch (or other suitable force sensor) is depressed, indicating the presence of an orthodontic appliance. The force sensor can be coupled to a timer, which records the amount of time the sensor is depressed (indicating that the orthodontic appliance is in the patient's mouth), or not depressed (indicating that the orthodontic appliance is not in the patient's mouth). Either type of data can be used to determine a patient's compliance with a treatment program.

In an illustrative embodiment of a system configured to determine compliance with use of one or more orthodontic appliances, the compliance device (e.g., compliance device 4220A, 4220B, 4220C, 4225, 4530) that is bonded, permanently or removably, to a patient's tooth can detect when an orthodontic appliance (e.g., an aligner or other removable appliance) is disposed within the patient's mouth. The detection can be based on a change in capacitance, an optical property (or change in optical property, the optical property can include, but is not limited to, threshold detection of light, interruption in light detection, threshold light reflection), force or pressure (or a change thereof), a magnetic field (or change in magnetic field), or the like. The compliance device can stores data associated with the detection, including data that the detection occurred, time and/or date data, or the like. In some embodiments, the compliance device detects and stores information associated with the presence or absence of the orthodontic appliance at predetermined intervals over a period of time (e.g., up to about 24 hours, up to about 48 hours, or the like). The data indicate a first time during the period of time that the orthodontic appliance is detected by the compliance device (as described herein). The data can indicate a second time during the period of time, subsequent the first time that the orthodontic appliance is not (or is no longer) detected by the compliance device. The data can indicate a third time during the period of time that is subsequent the first time and prior to the second time and that is the last instance of detection by the compliance device of the orthodontic appliance prior to the second time. If the duration from the first time to the third time is equal to or exceeds a prescribed duration during which the patient was to wear or use the orthodontic appliance, a determination can be made that the patient complied with the orthodontic treatment program. If the compliance device does not detect the orthodontic appliance during the time period or if the duration from the first time to the third time is less than a prescribed duration during which the patient was to wear or use the orthodontic appliance, a determination can be made that the patient failed to comply with the orthodontic treatment program. In some embodiments, a percentage of compliance can be calculated.

In some embodiments, the compliance device can calculate a cumulative duration of orthodontic usage (and/or duration of absence of detection of the orthodontic appliance) during the time period based on the stored. In some embodiments, the compliance device is configured to compare the calculated cumulative duration with a prescribed duration of orthodontic appliance usage for the period of time pursuant to a prescribed orthodontic treatment program to assess or determine compliance.

When compared to a treatment/therapeutic protocol associated with use of the orthodontic appliance, compliance information associated with use of the orthodontic appliance can be generated. In some embodiments, the compliance information can include an indication that the patient has or has not complied with the treatment program, a percentage associated with the patient's compliance (and/or non-compliance), an amount of time of appliance usage over or under a target goal according to the protocol, a graphical or other representation of the patient's compliance (and/or non-compliance) over a predetermined time period, an indication of compliant or non-compliant time periods or instances within a predetermined time period, or the like. For example, in some embodiments, a patient is prescribed to full-time wear of an orthodontic appliance, which can include a goal for the orthodontic appliance to be worn or used by the patient for 22 hours per day. The treatment protocol can be set to default values or customized by the clinician. The compliance information can indicate a patient's percentage of compliance with the protocol. For example, if the patient wears the appliance for 20 hours for one day, and the patient's goal is 22 hours for that day, then the patient's percentage of compliance is about 90%. If the patient wears the appliance for 24 hours for another day, and the patient's goal for that day is 22 hours, then the patient's percentage of compliance is about 110%. In another example, the compliance information can be represented in a difference of the actual time of appliance usage versus a target duration. Using the foregoing examples, if the protocol specifies a target appliance usage of 22 hours per day, and the patient's actual usage is 20 hours one day, the compliance information can reflect −2 hours (or a usage of 2 hours less than the goal). Similarly, if the protocol specifies a target appliance usage of 22 hours per day, and the patient's actual usage is 24 hours another day, the compliance information can reflect +2 hours (or a usage of 2 hours more than the goal).

In some embodiments, the generation of compliance information can account for or disregard a data point as being unrelated to compliance. In some embodiments, variations in temperature can occur as a result of normal user activities. For example, a sudden change in temperature (e.g., having a rate of temperature change greater than a predetermined threshold, and/or a spike up or down in temperature for a duration shorter than a predetermined duration) can indicate that the user is consuming a hot or cold beverage or afflicted with an abnormal condition that raises the user's body temperature to an above- or a below-normal value. The generation of compliance information can be configured to disregard data associated with such a sudden change in temperature as being non-relevant to the compliance determination. Similarly, during use, an orthodontic appliance can be temporarily removed from or displaced within the patient's mouth such that the sensor does not detect the orthodontic appliance for a brief time period. This can occur, for example, when the appliance is adjusted within the user's mouth for comfort, or is temporarily (e.g., less than about 1 minute, about 3 minutes, or less than about 5 minutes) removed, e.g., for a brief cleaning of the appliance or brushing of the patient's teeth, or the like). The generation of compliance information can be configured to disregard data associated with a lack of detection of the orthodontic appliance during the temporary removal as being non-relevant to the compliance determination. In some embodiments, such as where the compliance monitoring device comprises a force or pressure sensor, the sensor can distinguish between the force applied by an aligner, and a force applied unrelated to compliance. In order to distinguish between applications of forces not associated with the presence of an orthodontic appliance (e.g., brushing teeth, eating, tongue or cheek movement, etc.), the force sensor can be activated only after the sensor (or a portion of the sensor) is depressed for a certain amount of time (e.g., 3 seconds, 5 seconds, 10 seconds, or any suitable amount of time) to begin recording an amount of time an appliance is in place (or, alternatively, stop logging the amount of time an appliance is not in place).

The compliance information generation, or any processing (e.g., determination, assessment, calculation, or the like) of compliance related data (e.g., input data, time and/or date data, or the like) described herein, can be executed by the compliance device, the light therapy apparatus, or the external device, or a combination thereof.

In some embodiments, the compliance device is configured to calculate at least a portion of the compliance information. For example, in some embodiments, the compliance device optically receives an indication of the orthodontic treatment program associated with use of the orthodontic appliance, and determines at least a portion of the compliance information based on orthodontic treatment program specifications and based on the received input (e.g., temperature, pressure, force, etc.) and, optionally, date/time data. In such embodiments, the compliance device can be configured to transmit an indication of the compliance information to the data communication device, in addition or as an alternative to the signal associated with the input and/or time/date data.

In some embodiments, the data communication device is configured to calculate at least a portion of the compliance information, in addition to or instead of the compliance device and/or the external device. For example, in some embodiments, the data communication device optically receives information associated with the orthodontic treatment program (including, for example, a prescribed or expected use of the orthodontic appliance), and calculates or otherwise generates the compliance information based on the orthodontic treatment program specifications and based on the received input or orthodontic appliance detection data and, optionally, date/time data. The data received by the data communication device from the compliance device can be “raw” or source (e.g., unprocessed) data.

In some embodiments, the external device is configured to calculate at least a portion of the compliance information, in addition to or instead of the compliance device and/or the data communication device. The external device can be or include for example, a Smartphone, a tablet, a personal computer, and/or any other suitable electronic device. The external device can be configured to be in electrical communication with the data communication device. For example, in some embodiments, the data communication device can wirelessly (e.g., via Bluetooth®) transmit a signal including data associated with the input (e.g. temperature, force, pressure, etc.) and, optionally, the date and/or time, received from the compliance device, to the external device. In another embodiment, the data communication device can transmit a signal to the external device via a wired connection (e.g., via a USB drive). The external device can be configured to calculate, assess, or compare the data included in the signal such that the external device can generate the compliance information based on specifications associated with the orthodontic treatment program and based on the received data.

Use with Multiple Aligners

As disclosed herein, aspects of the embodiments disclosed herein are particularly useful when one or more orthodontic appliances, e.g., as part of a set, are employed by the user over time (e.g., in embodiments in which the orthodontic treatment program specifies appliance progression to occur). In such embodiments, whether the same compliance device is used across multiple orthodontic appliances or a new compliance device is employed for each orthodontic appliance, each compliance device can be operated in one or more of the modes described herein, and the compliance information generated can account for the progression of orthodontic appliances. For example, the user can communicate an indication of progression or switching orthodontic appliances to a data communication device (e.g., directly, or via a computer or other electronic device in communication with the light therapy apparatus), and the data communication device can be configured to associate physiological parameter data, time/date data, and/or compliance information received from the compliance device thereafter with use of the next orthodontic appliance in a set of orthodontic appliances. In other embodiments, each orthodontic appliance in the set of orthodontic appliances and/or each compliance device of one or more compliance devices, e.g., as part of a set, can be configured to provide an identifier or signal to the data communication device identifying or otherwise indicative of which orthodontic appliance and/or compliance device is in use in the patient's mouth.

In some embodiments, an orthodontic treatment program describes, e.g., prescribes, a successive or sequential use of one or more orthodontic appliances from a set of orthodontic appliances, each designed to effect tooth movement in a progressive manner (e.g., “appliance switching” or “appliance progression,” as described and referred to herein). The system is useful for monitoring patient compliance with the prescribed orthodontic appliance, including during an orthodontic treatment program that includes appliance progression (or successive use of multiple orthodontic appliances during the orthodontic treatment program).

In some embodiments, for example where appliance progression is prescribed, the system can comprise a set of compliance devices. The set of compliance devices can comprise one or more active compliance devices. The set of compliance devices can optionally also comprise one or more passive or placebo compliance devices. The placebo compliance device can be devoid of one or more components for detecting input (e.g., physiological parameter, or temperature) data, or can otherwise be configured not to detect the input (e.g., physiological parameter, or temperature). The placebo compliance device is configured to be visually indistinguishable to the patient by casual observation (e.g., during typical use of the device). In this manner, in use, the patient is expected to be unaware of whether the active compliance device or the placebo compliance device is being used with respect to a particular orthodontic appliance, such that the presence of placebo compliance device does not negatively affect the patient's intent to comply with using the orthodontic appliance pursuant to the predetermined orthodontic treatment program.

In such embodiments, for example, when a data communication device such as the light therapy apparatus or the probe receives no communication or otherwise does not receive a signal associated with the input (e.g., physiological parameter, temperature, pressure, force etc.) data or time/date data associated with the input at a second time from a compliance device (e.g., a second compliance device) associated with a second orthodontic appliance after the data communication device has received data (e.g., input and/or time/date data), for example at a first time before the second time, from a first compliance device (e.g., compliance device) associated with a first orthodontic appliance (e.g., appliance) during a patient's orthodontic treatment program, the data collection device can be configured to determine whether a placebo compliance device is being used with the second orthodontic appliance. In such an embodiment, the data collection device can be configured to produce an indication of or otherwise determine that the user is being compliant with the predetermined (second) orthodontic appliance usage.

In another example, an external device or a clinician/physician can determine whether a placebo compliance device is being used based in part on data received from the compliance device(s) and/a data collection device, and optionally based in part on a predetermined schedule or order of active and placebo compliance devices.

In this manner, because the user might be unaware of which compliance devices (if any) are placebo devices, user compliance with the orthodontic treatment program can be improved while reducing overhead manufacturing costs associated with compliance monitoring (e.g., due to cost savings achieved by a reduction in components for the placebo compliance devices).

Although the compliance device can be configured for use with a single orthodontic appliance, in some embodiments, the compliance device can be used with multiple orthodontic appliances. For example, the compliance device, when bonded to the patient's tooth, can be operatively coupled to a progression of orthodontic appliances, each of which can be configured to activate the sensor of and/or wirelessly communicate with the compliance device. In another example, the compliance device can be removed from a first orthodontic appliance from a set of orthodontic appliances and coupled to a second orthodontic appliance from the set of orthodontic appliances (e.g., when the user progresses through appliances). In some embodiments, the system can comprise a set of compliance devices, such that a user can attach a new compliance device to each orthodontic appliance from the set of orthodontic appliances (e.g., at the time of initial use during the orthodontic treatment program of a particular orthodontic appliance from the set of orthodontic appliances).

### Methods

Methods described herein relate to the use of compliance monitoring devices to track use of orthodontic systems (e.g., orthodontic appliances, light therapy systems, etc.). Methods can comprise one or more of manufacturing a compliance monitoring device, adhering a compliance monitoring device to a user, adhering a compliance monitoring device to an orthodontic appliance, collecting compliance data using the compliance monitoring device, re-charging a power source of the compliance monitoring device (such as with a light therapy apparatus), communicating data collected by the compliance monitoring device to a data collection device (such as a probe or light therapy apparatus), determining a user's compliance with the use of an orthodontic appliance and/or light therapy apparatus, and/or removing a compliance monitoring device.

Methods for Making Compliance Monitoring Devices

Methods described herein can comprise making compliance monitoring devices configured to be placed in the mouth of a user. As described herein, a compliance monitoring device can comprise one or more various components, including a processor, transceiver, memory, power source, sensor, or any other suitable component. One or more of the components of a compliance monitoring device can comprise circuitry and/or one or more microchips. Thus, methods for making a compliance device can comprise configuring one or more circuit boards and/or microchips. Methods can also comprise placing one or more components inside a casing. In some embodiments, method can comprise encapsulating one or more components of a compliance monitoring device in a biocompatible material, such as, for example, a biocompatible plastic.

Methods for Adhering Compliance Monitoring Devices

Methods described herein can also comprise attaching compliance devices to a tooth of a user, or to an orthodontic appliance such as an aligner. In some embodiments, attaching a compliance device to a tooth can comprise the use of an adhesive (such as an epoxy or resin, as described herein). In some embodiments, light, such as blue light, can be used to cure the adhesive (e.g., to harden it to affix the compliance device to the tooth). A compliance monitoring device can be attached to a tooth in any suitable manner. For example, in some embodiments, methods for attaching a compliance device to a tooth of a user can comprise the use of an attachment template, as described herein. A user can place the compliance monitoring device in a protrusion of an attachment template, place an adhesive on the tooth-contacting side of the compliance monitoring device, and place the attachment template over the teeth of a patient. In some embodiments, methods for attaching a compliance monitoring device can comprise leaving the attachment template on the tooth of a user until the adhesive dries/hardens. In some embodiments, methods for attaching a monitoring device can further comprise manipulating the attachment template to secure the compliance device to the tooth, and/or to remove the attachment template without disturbing the placement of the compliance device. For example, attachment templates can be flexible such that a user can be able to apply pressure to the compliance device through the attachment template in order to facilitate adhesion to a patient's tooth. In some embodiments, attachment templates be flexible such that a user can first remove the portion of the attachment template in contact with the compliance device from contact (such as by stretching a portion of the template) such that removal of the attachment template does not disturb the placement of the compliance device.

In some embodiments, methods for attaching a compliance device to a tooth do not comprise using an attachment template. Compliance monitoring devices can be adhered to the tooth of a user, or an orthodontic appliance, by placing an adhesive on the compliance device, and/or the surface of the tooth or appliance, and contacting the compliance device to the surface. In some embodiments, methods for contacting the compliance device to the surface of a tooth or orthodontic appliance comprise using a specialized tool, such as an instrument or probe configured to hold the compliance device while it is attached to the surface of a tooth or aligner.

In some embodiments, methods for attaching a compliance device can comprise modifying a surface of a tooth, orthodontic appliance, or compliance monitoring device. For example, in some embodiments, a user can etch, make a groove in, make a hole in, make a recess in or otherwise modify a tooth, orthodontic appliance or compliance monitoring device (or any combination thereof), in some embodiments a surface thereof, to facilitate adhesion of the compliance monitoring device to the tooth, orthodontic appliance or compliance device, or surface thereof. In some embodiments, magnets can be used to couple the compliance monitoring device to a surface. In such embodiments, methods for attaching a compliance device can comprise painting or otherwise adhering a magnetic material onto a surface (e.g., of a tooth, orthodontic appliance, compliance device, etc.).

In some embodiments, where compliance monitoring devices are attached to an orthodontic appliance, methods can comprise placing a compliance monitoring device within an orthodontic appliance. For example, in some embodiments, methods can comprise molding a compliance device into an orthodontic appliance, placing a compliance monitoring device into a pocket or recession in an orthodontic appliance, or any suitable method.

Methods for Recharging Compliance Devices

As described herein, compliance monitoring devices can comprise a rechargeable power source. Thus, methods can comprise recharging the compliance monitoring device. In some embodiments, recharging the compliance monitoring device can comprise bringing a light source (e.g., a light source that emits visible light at a prescribed wavelength) into close proximity with the compliance monitoring device such that a transceiver of the device can receive the light and convert it into charge. In some embodiments, the light source can be a light source of a light therapy apparatus, such as those described herein. Methods for recharging the power source of a compliance monitoring device can therefore comprise placing a light therapy device in the mouth of a patient. In some embodiments, the light therapy source can automatically activate a charging mode of the compliance monitoring device. For example, the compliance monitoring device can be activated upon receipt of light at a particular wavelength, or, the light therapy apparatus can send a wireless communication to the compliance monitoring device to activate the charging mode (e.g., when in close proximity to the device). In other embodiments, a charging mode of the compliance device can be activated by a user. For example, a user can operate a mobile application to operate a charging mode. Although a light therapy apparatus is primarily referred to herein as a method for recharging a power source of a compliance monitoring device, any suitable method can be used to recharge the battery of a compliance monitoring device. For example, in some embodiments, the compliance monitoring device can be charged via an external light source not associated with a light therapy apparatus. The external light source can be a separate component, or can be coupled to a data communication device such as the probe described herein. In other embodiments, the compliance device power source can be recharged by other methods for wireless charging, such as, for example, Bluetooth, or the like. The compliance device power source can be configured to be recharged using one or more of the following: mechanical motion of a user's jaw movements (e.g., chewing), bioenergy battery systems such as enzymatic glucose biofuel cells, photovoltaic energy (e.g., from ambient light, from therapeutic light sources, and/or from a light source dedicated for recharging), and electromagnetic energy (e.g., from ambient electromagnetic fields and/or intentional electromagnetic fields such as those generated by wireless charging transmitters).

Methods for Communicating Data

Methods can further comprise communicating data to and from a compliance monitoring device. In some embodiments, collecting/transmitting data from/to a compliance monitoring device can comprise bringing an external device in close proximity to a compliance monitoring device. For example, a receiver, a transmitter, or transceiver on a probe or a light therapy apparatus can be configured to collect or transmit data from or to a compliance monitoring device. In some embodiments, a compliance monitoring device is configured to communicate with and/or collect and/or transmit data from and/or to a receiver, transmitter, or transceiver on other external dental equipment, such as a digital optical scanner or a portion thereof. Methods can comprise bringing a probe or other external dental equipment comprising a receiver or transceiver in close proximity (e.g., near or in the mouth of a user) to a compliance monitoring device. In some embodiments, bringing a probe or other dental equipment in close proximity to the compliance monitoring device (e.g., in contact with, from about 0 mm to about 5 mm, from about 0 mm to about 10 mm, from about 0 mm to about 20 mm, from about 1 mm to about 20 mm, from about 5 mm to about 20 mm, from about 10 mm to about 20 mm) can prompt the compliance monitoring device to automatically offload data to the probe or other dental equipment.

The probe other dental equipment can activate the data communication mode of the compliance monitoring device with a light pulse, to trigger the compliance monitoring device to offload data. As described herein, the compliance monitoring device can transmit data via an LED. The probe or other dental equipment can “interrogate” the compliance monitoring device via encoded light pulses in order to receive the data collected by the sensor (e.g., with an electronic handshake). For example, the probe or other dental equipment can send a first light pulse to “wake” the compliance device and place the compliance device in state in which data can be transferred from the compliance device. The probe or other dental equipment can then transmit light pulses to the compliance device that the compliance device can recognize as bona fide or genuine. In some embodiments, the probe or other dental equipment can send a single light pulse sequence to initiate and/or achieve one or more of the following: “wake” the compliance device, place the compliance device in a state in which data can be transferred from the compliance device, and confirm that the pulses are genuine. Once the compliance device receives the light pulses and recognizes and/or verifies them as genuine, the compliance device can send responsive light pulses to the probe or other dental equipment to transfer the data collected by compliance monitoring device. The compliance monitoring device, can, for example, communicate the amount of time that the sensor registered an orthodontic appliance as in the patient's mouth, or not in the patient's mouth. The probe or other dental equipment can communicate with any suitable component of the compliance monitoring device (e.g., memory, processor, transceiver, receiver, transmitter or any combination thereof). In some embodiments, the probe or other dental equipment can send a light pulse (or light pulse sequence) confirming receipt of the data from the compliance monitoring device. Additionally or alternatively, at least some, and in some embodiments all, of the data can be erased from the compliance monitoring device after it is transferred.

In some embodiments, the light therapy apparatus can collect data from the compliance device. The light therapy apparatus can operate in a similar manner as the probe to collect data from the compliance monitoring device, as described herein. That is, the light therapy apparatus can activate the communication mode of the compliance device (e.g., with a light pulse), the light therapy apparatus can interrogate the compliance monitoring device, and the compliance monitoring device can communicate (e.g., via a LED) the amount of time that the sensor registered an orthodontic appliance either in or out of the patient's mouth.

In some embodiments, a data collection device can be coupled to an aligner. For example a data collection device can be a small, inconspicuous, computational device that is configured to be coupled to a portion of the aligner. The data collection device can comprise a transceiver, memory, power supply, processor, and any other suitable components. The compliance monitoring device can be configured to communicate with a data collection device coupled to an aligner in any suitable manner (e.g., light, Bluetooth, or any suitable form of wireless communication.).

In other embodiments, data collection device can be external computational devices. For example, a compliance monitoring device can be configured to wirelessly communicate with a computational device, such as a mobile phone or a computer, or a cloud-based data storage system. The compliance monitoring device can communicate data to an external computational system using any suitable wireless communication method (e.g., Bluetooth, Near filed communication (NFC) or the like).

In some embodiments, after the compliance monitoring device has communicated information to a data collection device, the memory of the compliance monitoring device can be cleared of some or all data. For example, it might be the case that the compliance device has a limited memory capacity. Thus, clearing the memory of some or all data can make space for more data collection and storage by the compliance monitoring device. This ability can aid in permitting the compliance device to be small and inconspicuous in the mouth of a user.

Methods can further comprise transferring data from a data collection device to an additional computational device for further processing. For example, a probe or light therapy apparatus can collect data from a sensor, and subsequently offload data to a processor in a computational device (e.g., a mobile device, computer, cloud-based storage system, etc.). This can provide the benefit of transmitting data to a more powerful system for processing and determining compliance information, as described below. Further, it can be more convenient for users (such as patients, orthodontists, doctors, etc.) to view either raw or processed data on a personal computational device (e.g., a mobile phone, laptop computer, or other personal computer).

Methods for Determining Compliance

Some embodiments include methods for determining a patient's compliance with use of an orthodontic appliance, as described herein. In some embodiments, the methods can be implemented or executed by one or more components of any suitable compliance monitoring system described herein (e.g., system 4500). The methods can comprise allowing a data communication device to receive from a device a signal. The data collection device can comprise an emitter, in some embodiments a plurality of emitters, each configured to be disposed within a patient's mouth. The device can be configured to be disposed within a patient's mouth and to detect an input a first time and a second time, the signal including data detected at the first time and second time. For example, in some embodiments, the data collection device can optically receive a light signal that includes sensor input data and/or time/date data. The data included in the signal can be, for example. “raw” or source data (e.g., unprocessed data). The sensor input data can include one or more physiological parameters, force, pressure, etc. The sensor input data can include detection of an orthodontic appliance, such as by activation of the sensor by the orthodontic appliance or any other suitable input (e.g., force, light, or the like) described herein. The signal that includes the sensor input data (e.g., one or more physiological parameters, orthodontic appliance detection such as via force/pressure, or the like) and/or time/date data is received from a compliance monitoring device configured to be disposed within a patient's mouth and further configured to detect the input and/or time/date information. In some embodiments, the signal is included in light transmitted from the device to the light therapy apparatus.

Methods for determining patient compliance can comprise generating compliance information based on the signal. The compliance information can be associated with use of an orthodontic appliance, which can be coupled to the compliance device or to the tooth of a user. In some embodiments, the data communication device (e.g., light therapy apparatus, probe, etc.) generates the compliance information and transmits an indication of the compliance information to a computer or external electronic device. In some embodiments, the external electronic device is configured to process the data included in the signal to generate compliance information. For example, in some embodiments, the light therapy apparatus wirelessly transmits (e.g., via Bluetooth®, or the like) a signal that includes the detected input (e.g., force, pressure, temperature) and/or time/date data to the computer or external electronic device, which generates the compliance information.

In some embodiments, the orthodontic appliance is a first orthodontic appliance from a plurality, e.g., set, of orthodontic appliances and the compliance device is a first compliance device from a plurality, e.g., set of compliance devices. In some embodiments, the methods can comprise optically receiving, at the data communication device, from a second compliance device associated with a second orthodontic appliance from the set of orthodontic appliances, a signal (or other indication) of an input (e.g., a physiological parameter such as temperature, force, pressure, etc.) and/or time/date information detected by the second compliance device. In this manner, aspects of the methods are useful for compliance monitoring when multiple orthodontic appliances are successively employed (e.g., when appliance or aligner, switching or progression occurs).

In some embodiments, the compliance device comprises a first emitter that optically communicates the signal to the data communication device. In such embodiments, the signal can be optically received at a second emitter associated with the data communication device. In some embodiments, the method can comprise charging or recharging the compliance device, as described herein.

Methods can also comprise removing a compliance monitoring device from the tooth of a user. In some embodiments, removing a compliance device from a tooth of a user can comprise manually applying pressure to the device to detach it from the tooth. For example, a user can use a tool (such as pliers) to remove the device from the tooth. A user can additionally or alternatively wedge a tool between the compliance device and the tooth to pry the device off of the tooth. In some embodiments, the adhesive used to attach the compliance device to the tooth is dissolvable or abradable under certain conditions. Thus, detaching the compliance device from the tooth can involve the use of a substrate (e.g., by rubbing the substrate over the compliance device and/or circulating the substrate within the mouth of the user). In some embodiments, a sanding device can be used to remove the compliance monitoring device. A sanding device (e.g., a dental hand piece or bur) may be used in conjunction with any of the herein described methods to remove any residual adhesive left on the patient's tooth after the compliance device is removed. In other embodiments, the adhesive can be dissolved by an aqueous or nonaqueous solvent, or mixture thereof.

### EXAMPLES

The invention is further described with reference to the following specific examples, which are not meant to limit the invention, but rather to further illustrate it.

### Example 1

A patient presents with a 7 mm overjet between her maxillary central incisor and mandibular central incisor (i.e., the mandibular bone is retrusive). A Herbst appliance is fitted on upper and lower molars of the patient. The appliance is adjusted to provide edge-to-edge incisal positioning of the upper and lower incisors. Typically, the appliance is fitted and remains in place for about 12 months.

Light is administered transdermally to the patient's right and left temporomandibular joint. The light is administered using a light array which contacts the patient's face and which irradiates light having a wavelength of about 850 nm and an intensity of about 50 mW/cm². A first light array which contacts the patient's face administers light to the right temporomandibular joint and a second light array which contacts the patient's face administers light to the left temporomandibular joint. This light treatment is administered on a daily basis for about 20 minutes, over the patient's entire skin overlying the right and left temporomandibular joints. The light arrays are positioned and held at the desired location by a head set which uses the bridge of the patient's nose, and the patient's two cars, to maintain a repeatable position throughout the treatment regimen, over multiple sessions. The head set is one set forth in one of FIGS. 8A-8D.

Treatment continues for 3 months, at which time the Herbst appliance is disengaged without being removed, allowing the lower jaw to freely close. The patient is subsequently recalled in 3 weeks to determine the precise degree of new overjet in order to confirm the result. If the overjet is stable, treatment is discontinued. If the overjet has not been fully corrected, the Herbst appliance is re-engaged and phototherapy is continued for an additional period of time, such as about two to three months.

### Example 2

A 12-year-old male presents with a Class 2 Division 1 malocclusion with an 8 mm overjet caused by insufficient mandibular growth in a horizontal and vertical direction and slight overgrowth horizontally of maxillary bone based on cephalometric analysis.

The patient is fitted with a mandibular anterior repositioning appliance (Mara functional appliance). The Mara is intended to posture the mandible forward about 8 to about 10 mm. This causes tension on the musculature of the mandible and the temporomandibular joint and stimulates bone remodeling and growth of condyle and glenoid fossa. At the same time a light therapy device, such as the device shown in FIG. 21A modified so that the light sources overlay the temporomandibular joint, is adjusted to register on the nose and ears of the patient. A light array of the device covers the entire anatomic area of the patient's temporomandibular joints and is positioned on the patient's face directly over the right temporomandibular joint and the left temporomandibular joint, contacting the skin and applying slight pressure to the tissue.

The light treatment regimen includes 30 minutes of light administered daily at a wavelength of about 850 nm continuous wave, at an intensity on the surface of the skin of about 50 m W/cm². The treatment is applied daily throughout the treatment of the Mara. At 4 months, the patient discontinues the light therapy and has the Mara de-activated by the dentist or other health-care professional so that the mandible is in a passive position. After two weeks, the patient returns and the dentist or other health-care professional assesses the now stable overjet. If the overjet is normal the Mara is removed and light therapy is permanently discontinued. If the overjet is still excessive, an additional time period of Mara and light therapy treatment is conducted for 2 months, and then re-assessed.

### Example 3

A male adult patient is tested for Vitamin D3 blood serum levels at the same time as his routine orthodontic examination and records appointment. The patient's diagnosis is Class I mild crowding with 4 mm of crowding on the upper arch and 4 mm on the lower arch. An orthodontic treatment plan is formulated to include the installation of a fixed orthodontic appliance with some mild expansion of the upper and lower arches. Laboratory results indicate that the patient's vitamin D3 serum levels are at 20 ng/ml, which is considered to be deficient.

The patient self-administers oral oil-based vitamin D3 capsules of 6000 IU per day for 3 months to increase his vitamin D3 serum levels. Laboratory serum testing is optionally performed again after 3 months of vitamin D3 supplementation. The patient maintains or adjusts his oral dose of vitamin D3 based on his subsequent lab results.

Orthodontic treatment is started either after the 3 month period or anytime prior. The patient has conventional fixed orthodontic brackets, and bands placed on his teeth with an initial 0.016 inch NiTi wire tied in place with silicone ligatures. Light is administered to the patient on a daily basis in all regions of the maxillary bone and mandibular bone for 20 minutes at an intensity of 50 mW/cm² at wavelength of about 850 nm. The light can be administered using a light therapy apparatus, such as the one shown in FIG. 21A. The orthodontic treatment continues with the finishing of teeth once the arches have been expanded. It is believed that the active orthodontic treatment will be completed in 50% to 75% less time due to the combination of daily administration of light and improved vitamin D3 serum status.

At a passive state of orthodontic treatment. i.e., retention phase, a fixed retention orthodontic appliance is installed on the patient's teeth. In one example, a Hawley retainer is a removable appliance that is designed to maintain tooth position of the anterior teeth. The Hawley retainer can be installed on the patient's anterior teeth. In some embodiments, a fixed retainer appliance is bonded to the lower 6 anterior teeth. The patient continues with vitamin D3 supplementation. In some examples, the patient self-administers 2000 IU per day to 12,000 IU orally per day. The dosage can be determined based on vitamin D blood serum levels which can be measured periodically to determine dosing. As a result, alveolar bone density around the teeth is increased during the passive phase. During the passive stage, the patient is administered with light once per week with a light therapy apparatus, such as the light therapy apparatus shown in FIG. 21A, in all areas of the upper and lower arch at a wavelength of about 625 nm.

### Example 4

An animal study was conducted to demonstrate the effect of light treatment and functional-appliance use on mandibular condylar growth. Twenty-four rats were divided equally into six groups of four animals: Group 1 received laser light treatment, but did not wear a functional appliance; Group 2 received laser light treatment and wore a functional appliance; Group 3 received LED light treatment, but did not wear a functional appliance; Group 4 received LED light treatment and wore the same type of functional appliance as Group 2; Group 5 wore the same functional appliance as Group 2, but did not receive light treatment, and served as the positive control group; and Group 6 did not wear a function appliance, did not receive light treatment and served as the negative control group. The rat functional appliance was analogous to a human functional appliance.

The rats of Groups 1-4 received their respective light treatment for ten minutes every day for four weeks. The rats of Groups 5-6 (the control groups) received “sham” treatment, i.e., no laser or LED light, during this time. The wavelength and intensity of the laser and LED light treatment was the same—850 nm at 10 mW/cm².

At the end of four weeks, the mandibles of each of the rats were analyzed and the surface areas of the cartilaginous layers of the condyles were measured. These layers include the fibro-cartilaginous (FL) layer, the proliferative layer (PL), the chondrocyte layer (CL), and the hypertrophic layer (HL).

The results showed that Groups 1 and 2 (laser light treatment) had a statistically significant increase in the FL compared to the control groups, Groups 5 and 6. The CL of Groups 3 and 4 were also found to have significantly increased as a result of the LED light treatment compared to the control groups, Groups 5 and 6. The HL of Group 2 was found to have a greater increase than Group 5. In other words, administering laser light in combination with wearing a functional appliance was more effective for increasing the HL than simply wearing a functional appliance. Lastly, the chondroblastic layer of Group 4 was found to have greater increase than that of Group 2. These results demonstrate that administering laser light in combination with wearing a functional appliance is useful for stimulating mandibular condylar growth, lengthening mandibular bone and, accordingly, regulating oral or maxillofacial bone remodeling.

### Example 5: Use of Functional Appliance and Light Therapy

In this example, a patient is fitted with a Herbst appliance that levels after the patient's upper teeth are aligned such that her mandible is able to advance to an ideal, unobstructed incisor overjet (OJ).

Pre-Herbst Activities

The patient's OJ and maximal protrusive mandible position are measured prior to administering light therapy. Upper and lower casts of the patient's bite are made, and photographs of her oral cavity, including side pictures, occlusal pictures and anterior pictures, are taken. The casts and photographs are retained. A Herbst appliance is constructed such that after it is installed in the patient her OJ will more protrusive than ideal, but less than edge-to-edge.

Day of Herbst Delivery

The Herbst appliance is cemented in the patient's oral cavity, and her OJ is optionally measured. The patient is fitted with an extra-oral light-therapy device having one or more light-source arrays that contact her face directly about one or both of her temporomandibular joints and is irradiated with light having a wavelength ranging from about 600 nm to about 900 nm, in a specific example, about 850 nm. One or more photographs of the positioned arrays are taken, and the patient is asked to report to her orthodontist any change in OJ. The extra-oral light therapy device is removed from the patient and the patient is instructed to use the extra-oral device on a daily basis for 20 minutes per day.

Post-Herbst Insertion

The patient returns to her orthodontist two weeks later for a follow-up visit and three months later to check the position of the appliance. At the three-month visit, shims that alter the length of the appliance's telescopic arms are optionally added to the appliance. At four months following appliance insertion, the appliance is deactivated and the patient's OJ and maximal protrusive mandible position are remeasured. Two weeks later the patient's OJ is checked, and if it is less than ideal, then the appliance is reactivated. Iterations are performed every two weeks until the earlier of (i) the time when patient's OJ is ideal and (ii) two months. After the earlier of (i) and (ii), the patient's OJ and maximal protrusive mandible position are remeasured, upper and lower casts of the patient's bite are made, photographs of her oral cavity are taken and the measurements and photographs are compared to those taken during the pre-Herbst activities.

### Example 6

A male adult patient's Vitamin D3 blood-serum level is measured during his routine orthodontic-examination and records appointment. Laboratory results indicate that the patient's vitamin D3 serum levels are at 20 ng/ml, which is considered to be deficient and abnormal. The patient's orthodontic diagnosis is Class I mild crowding with 4 mm of crowding on the upper arch and 4 mm on the lower arch. An orthodontic treatment plan is formulated to include the installation of a fixed orthodontic appliance with some mild expansion of the upper and lower arches.

The patient self-administers oral oil-based vitamin D3 capsules at an amount of 6000 IU per day for 3 months to increase and normalize his vitamin D3 serum levels. Laboratory serum testing is optionally performed again after 3 months of vitamin D3 supplementation. The patient maintains or adjusts his oral dose of vitamin D3 based on his subsequent lab results.

Orthodontic treatment is started either after the 3 month period or no more than about three months prior. The orthodontic treatment includes conventional fixed orthodontic brackets and bands placed on the patient's teeth using an initial 0.016 inch NiTi wire tied in place with silicone ligatures. Light is administered to the patient on a daily basis for 20 minutes at an intensity of 50 mW/cm² at wavelength of about 850 nm using an intra-oral light therapy apparatus, such as the one shown in FIG. 1. The orthodontic treatment continues with the finishing of teeth once the arches have been expanded. It is believed that the active orthodontic treatment would be completed in 50% to 75% less time than orthodontic treatment without light therapy due to the combination of daily administration of light and vitamin D3 supplementation.

At a passive stage of orthodontic treatment, i.e., retention phase, a fixed retention orthodontic appliance can be installed on the patient's teeth. For example, a Hawley retainer, which is a removable appliance that is designed to maintain tooth position of the anterior teeth, is installed on a patient's anterior teeth. Alternatively, a fixed retainer appliance, such as one including orthodontic brackets, is bonded to the lower 6 anterior teeth. The patient continues with vitamin D3 supplementation. In some examples, the patient self-administers 2000 IU per day to 12,000 IU orally per day. The dosage can be determined based on vitamin D blood serum levels which can be measured periodically to determine dosing. As a result, alveolar bone density around the teeth can be increased during the passive stage. During the passive stage, the patient is administered once per week with light having a wavelength of about 625 nm using an intra-oral light therapy apparatus, such as the light therapy apparatus shown in FIG. 23 and/or FIG. 28, in areas of the upper and lower arch.

### Example 7

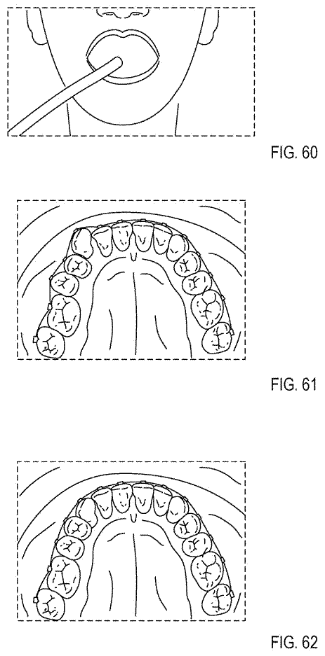

In one study, an illustrative intra-oral light-therapy apparatus of the invention, depicted in FIG. 60, was used during the alignment phase of orthodontic treatment to irradiate the maxillary anterior teeth of three (3) patients. The intra-oral light-therapy apparatus included an intra-oral housing with a light-emitting fabric panel embedded in each of a silicone buccal flange and a silicone lingual or palatial flange of the intra-oral housing, and a LED light source disposed exterior to the mouth of the patient. The fabric of the light-emitting panel was made by weaving acrylic optical fibers into a mat. Each patient was provided with his or her own intra-oral light-therapy apparatus, which was used in combination with a conventional buccal fixed orthodontic bracket treatment protocol. The treatment and results of three of the patients—Patient A, Patient B, and Patient C—are described in detail herein.

During the study, the intra-oral light-therapy apparatus was used by each patient every day until he or she achieved an LII value of 1 mm or less. When in use, the intra-oral light-therapy apparatus contacted each patient's maxillary alveolar soft tissue and irradiated the tissue with light having a wavelength of about 850 nm for about six minutes per day.

During the study, the patients visited a clinician every two (2) weeks. At each visit, the clinician performed regular orthodontic procedures, collected data that included intra-oral photographs and study models, recorded patient compliance and checked the functionality of the intra-oral light-therapy apparatus. To assess the effectiveness of the treatment, the clinician used the LII grading system to score the models produced at each appointment. Maxillary anterior intra-oral light therapy continued every day until the clinician determined that the patient's LII score decreased to 1 mm or less.

Patients were selected for participation in this study based on, at least, the following criteria: (1) the patient was eligible for full mouth fixed orthodontic treatment of the upper arch to correct crowding, misalignment and rotated teeth: (2) a presence of permanent dentition (i.e., permanent, or adult, teeth): (3) the upper-arch teeth had an LII from 5 mm to 12 mm, provided that no tooth was blocked out of alignment; and (4) the patient was from 12 to 45 years old. The following types of individuals were excluded from this study: (1) pregnant women: (2) individuals enrolled in another study with periodontally involved teeth: (3) individuals who use bisphosphonates: or (4) individuals with any compromised dental or medical conditions.

Patient A

Patient A is a thirteen (13)-year-old female who wore SPEED System™ brackets and 0.016 inch Supercable nickel titanium wires during the alignment phase of her orthodontic treatment. On day 1 of the study, Patient A's upper-arch LII was about 5.1 mm. FIG. 61 is an illustration of Patient A's upper arch on day 1 of the study. After only 30 days of using the intra-oral light-therapy apparatus for light treatment in combination with the SPEED System™ brackets and the wires, Patient A's LII was reduced to 0.5 mm. The rate of Patient A's tooth movement during that period was about 1.07 mm/week. FIG. 62 is an illustration showing the corrected orientation of the teeth in Patient A's upper arch on day 30 of the study.

Comparison to Control

The results for Patient A were compared to those from a 13-year-old control patient who also wore SPEED System™ brackets and 0.016 inch Supercable nickel titanium wires during the alignment phase of her orthodontic treatment, but who did not receive light therapy. The control patient had an upper-arch LII of about 5.2 mm on day 1. In contrast to the tooth-movement time for Patient A, it took 78 days for the control patient's upper-arch LII to reduce to 1 mm or less. The rate of the control patient's tooth movement during that period was only about 0.42 mm/week.

Patient B

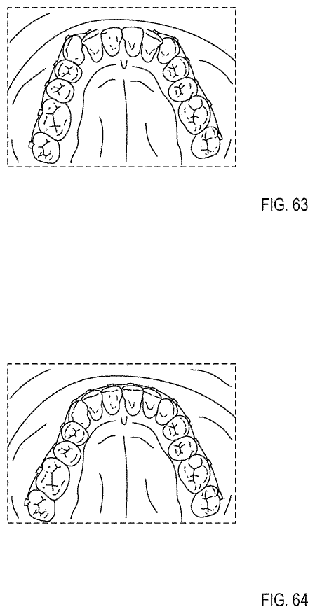

Patient B is also a thirteen (13)-year-old female who wore SPEED System™ brackets and 0.016 inch Supercable nickel titanium wires during the alignment phase of her orthodontic treatment. On day 1 of the study, Patient B's upper-arch LII was about 9.3 mm. FIG. 63 is an illustration of Patient B's upper arch on day 1 of the study. After only 41 days of using the intra-oral light-therapy apparatus for light treatment in combination with the SPEED System™ brackets and the wires, Patient B's LII was reduced to 0.8 mm. The rate of Patient B's tooth movement during that period was about 1.45 mm/week. FIG. 64 is an illustration showing the corrected orientation of the teeth in Patient B's upper arch on day 41 of the study.

Comparison to Control

The results for Patient B were compared to those from a similar-age, control patient who also wore SPEED System™ brackets and 0.016 inch Supercable nickel titanium wires during the alignment phase of orthodontic treatment, but who did not receive light therapy. The control patient had an upper-arch LII of about 8.8 mm on day 1. In contrast to the tooth-movement time for Patient B, it took 129 days for the control patient's upper-arch LII to reduce to 0.3 mm. The rate of the control patient's tooth movement during that period was only about 0.46 mm/week.

Patient C

Patient C is an eighteen (18)-year-old male who wore In-Ovation L Straightwire system brackets and both 0.012 inch and 0.016 inch nickel titanium wires during the alignment phase of his orthodontic treatment. On day 1 of the study, Patient C's upper-arch LII was about 5.02 mm. After only 42 days of using the intra-oral light-therapy apparatus for light treatment in combination with the In-Ovation L Straightwire system and the wires, Patient C's LII was reduced to zero. The rate of Patient C's tooth movement during that period was about 0.84 mm/week.

### Example 8

In one study, an intra-oral light therapy apparatus of the invention, depicted in FIGS. 65-66, was used during the alignment phase of orthodontic treatment to irradiate one or both of the maxillary and mandibular anterior teeth of nine (9) patients (Intra-Oral Group). The intra-oral light therapy apparatus included a flexible intra-oral housing with LEDs mounted on a flexible circuit and embedded in soft flexible buccal flanges of the intra-oral housing. Each patient was provided with his or her own intra-oral light therapy apparatus, which was used in combination with a conventional buccal fixed orthodontic bracket treatment protocol.

During the study, each patient wore 0.018 slot Mini-Diamond® brackets (obtained commercially from Ormco Corporation, Orange, Calif.) aligned with 0.014 or 0.016 inch nickel titanium wire, initially, and then progressed to using 0.016 inch by 0.016 inch (also referred to as “16×16”) nickel titanium wire.

During the study, the intra-oral light therapy apparatus was used by each patient every day until he or she achieved an LII value of 1 mm or less, with no single LII contact point greater than 0.25 mm. When in use, the intra-oral light therapy apparatus irradiated the tissue with light having a wavelength of about 850 nm (±5 nm). Unless otherwise noted herein, light therapy was administered to one or both of the maxillary arch or the mandibular arch of each patient for about three minutes per day, and at a light output intensity from about 60 mW/cm² to about 100 mW/cm². The mean intensity of the irradiated light for the Intra-Oral Group was about 70 mW/cm².

During the study, the patients visited a clinician every two (2) to three (3) weeks. At each visit, the clinician performed regular orthodontic procedures, collected data that included intra-oral photographs and study models, recorded patient compliance and checked the functionality of the intra-oral light therapy apparatus. To assess the effectiveness of the treatment, the clinician used the LII grading system to score the models produced at each appointment. The light therapy continued every day until the clinician determined that the patient's LII score decreased to 1 mm or less, with no single LII contact point greater than 0.25 mm. The clinician also collected dental impressions and models at T0 (i.e., day 1), which represents the day of maxillary bonding, start of orthodontic treatment (e.g., date brackets and/or wires are installed on the patient's teeth), date of patient assignment of the intra-oral light therapy apparatus, and start of the patient's daily usage of the intra-oral light therapy apparatus, and at T1, which represents the day at which the clinician determined the patient achieved an LII value of 1 mm or less, with no single contact point greater than 0.25 mm.

Patients were selected for participation in this study based on, at least, the following criteria: (1) the patient was eligible for full mouth fixed orthodontic treatment of the upper arch (and/or the lower arch) to correct crowding, misalignment and rotated teeth: (2) a presence of permanent dentition (i.e., permanent, or adult, teeth); (3) the upper-arch (and/or the lower-arch) teeth had an LII from 3 mm to 12 mm, provided that no tooth was blocked out of alignment; and (4) the patient was from 11-27 years old. The following types of individuals were excluded from this study: (1) pregnant women; (2) individuals enrolled in another study with periodontally involved teeth; (3) individuals who use bisphosphonates; or (4) individuals with any compromised dental or medical conditions.

The mean rate of tooth movement during the study period for the nine patients using the intra-oral light therapy apparatus in combination with the conventional buccal fixed orthodontic bracket treatment protocol, as described herein, was about 1.32 mm per week. The mean time to alignment for the nine patients was 41 days. The rates of tooth movement among the nine patients during the study period ranged from 0.42 mm per week to 2.35 mm per week, as shown in FIGS. 59 and 60. The treatment and results of the nine patients—Patient D, Patient E, Patient F, Patient G, Patient H, Patient I, Patient J. Patient K, and Patient L—are described in detail herein.

For any age herein that is represented to the nearest tenth, the age indicates the age in years and fraction of the year.

Patient D

Patient D is a 12.8-year-old male. On day 1 (i.e., T0) of the study, Patient D's upper-arch LII was about 5.7 mm. Light therapy was administered to Patient D's maxillary arch for about three minutes per day, and at a light output intensity of about 67 mW/cm². The patient received a light therapy dose of 12.1 J/cm² per day. After only 17 days of using the intra-oral light therapy apparatus for light treatment in combination with the Mini-Diamond® brackets and wires, Patient D's LII was reduced to 0 mm. The rate of Patient D's tooth movement during the study period was about 2.35 mm/week.

Patient E

Patient E is a 12.6-year-old female. On day 1 (i.e., T0) of the study, Patient E's upper-arch LII was about 6.0 mm. Light therapy was administered to Patient E's maxillary arch for about three minutes per day, and at a light output intensity of about 78 mW/cm². The patient received a light therapy dose of 14.0 J/cm² per day. After only 21 days of using the intra-oral light therapy apparatus for light treatment in combination with the Mini-Diamond® brackets and the wires, Patient E's LII was reduced to 0 mm. The rate of Patient E's tooth movement during the study period was about 2.0 mm/week.

Patient F

Patient F is a 13.2-year-old female. On day 1 (i.e., T0) of the study, Patient F's upper-arch LII was about 3.6 mm. Light therapy was administered for about three minutes per dental arch two times per day, and at a light output intensity of about 61 mW/cm². The patient received a light therapy dose of 22.0 J/cm² per day. After only 40 days of using the intra-oral light therapy apparatus for light treatment in combination with the Mini-Diamond® brackets and the wires, Patient F's LII was reduced to 0 mm. The rate of Patient F's tooth movement during the study period was about 0.63 mm/week. It is noted that Patient F missed a scheduled visit with the clinician on day 21, which may have resulted in the foregoing alignment rate being significantly underestimated.

Patient G

Patient G is a 14.3-year-old male. On day 1 (i.e., T0) of the study, Patient G's upper-arch LII was about 12.1 mm. Light therapy was administered for about three minutes per day, and at a light output intensity of about 78 mW/cm². The patient received a light therapy dose of 14.1 J/cm² per day. After only 50 days of using the intra-oral light therapy apparatus for light treatment in combination with the Mini-Diamond® brackets and the wires, Patient G's LII was reduced to 0 mm. The rate of Patient G's tooth movement during the study period was about 1.69 mm/week.

Patient H

Patient H is a 16.5-year-old female. On day 1 (i.e., T0) of the study, Patient H's upper-arch LII was about 5.5 mm. Light therapy was administered to Patient H's maxillary arch for about three minutes per day, and at a light output intensity of about 63 mW/cm². The patient received a light therapy dose of 11.3 J/cm² per day. After 92 days of using the intra-oral light therapy apparatus for light treatment in combination with the Mini-Diamond® brackets and wires, Patient H's LII was reduced to 0 mm. The rate of Patient H's tooth movement during the study period was about 0.42 mm/week. It is noted that Patient H missed a scheduled visit with the clinician on day 74, which may have resulted in the foregoing alignment rate being significantly underestimated.

Patient I

Patient I is a 14.2-year-old female. On day 1 (i.e., T0) of the study, Patient I's upper-arch LII was about 11.0 mm. Light therapy was administered to Patient I's maxillary arch for about three minutes per day, and at a light output intensity of about 78 mW/cm². The patient received a light therapy dose of 14.0 J/cm² per day. After 53 days of using the intra-oral light therapy apparatus for light treatment in combination with the Mini-Diamond® brackets and wires, Patient I's LII was reduced to less than 1 mm. The rate of Patient I's tooth movement during the study period was about 1.45 mm/week. It is noted that Patient I missed a scheduled visit with the clinician on day 21, which may have resulted in the foregoing alignment rate being significantly underestimated.

Patient J

Patient J is a 12.4-year-old female. On day 1 (i.e., T0) of the study, Patient J's upper-arch LII was about 5.5 mm. Light therapy was administered to Patient J's maxillary arch for about three minutes twice per day, and at a light output intensity of about 57 mW/cm². The patient received a light therapy dose of 20.4 J/cm² per day. After 49 days of using the intra-oral light therapy apparatus for light treatment in combination with the Mini-Diamond® brackets and wires, Patient J's LII was reduced to 0 mm. The rate of Patient J's tooth movement during the study period was about 0.79 mm/week.

Patient K

Patient K is a 13.9-year-old male. On day 1 (i.e., T0) of the study, Patient K's upper-arch LII was about 14.2 mm. Light therapy was administered to Patient K's maxillary arch for about three minutes twice per day, and at a light output intensity of about 58 mW/cm². The patient received a light therapy dose of 20.7 J/cm² per day. After 50 days of using the intra-oral light therapy apparatus for light treatment in combination with SPEED System™ brackets and wires, Patient K's LII was reduced to 0.8 mm. The rate of Patient K's tooth movement during the study period was about 1.88 mm/week.

Patient L

17.7-year-old female. On day 1 (i.e., T0) of the study, Patient L's lower-arch LII was about 3.0 mm. Light therapy was administered to Patient L's mandibular arch for about three minutes per day, and at a light output intensity of about 80 mW/cm². The patient received a light therapy dose of 14.4 J/cm² per day. After 22 days of using the intra-oral light therapy apparatus for light treatment in combination with the Mini-Diamond® brackets and wires, Patient L's LII was reduced to 0.8 mm. The rate of Patient L's tooth movement during the study period was about 0.70 mm/week.

Comparison to Controls

The results for Patients D-J were compared to those from two control patients, identified here as Control A and Control B. Controls A and B were selected, in part, for having a similar age and initial LII of the upper-arch as Patients D-J. The same clinician evaluated Patients D-J and Controls A and B.

Control A is a 14-year-old female control patient who wore SPEED System™ brackets, and wires as described for Patients D-J, during the alignment phase of her orthodontic treatment, but who did not receive light therapy. Control A had an upper-arch LII of about 5.9 mm on day 1. In contrast to the tooth-movement time for Patients D-L, it took 92 days for Control A's upper-arch LII to reduce to 0 mm. The rate of the Control A's tooth movement during that the study period was only about 0.45 mm/week.

Control B is an 11-year-old female who also wore one of SPEED System™ brackets, and wires as described for Patients D-J, during the alignment phase of her orthodontic treatment, but who did not receive light therapy. Control B had an upper-arch LII of about 6.6 mm on day 1. In contrast to the tooth movement time for Patients D-L, it took 105 days for Control B's upper-arch LII to reduce to 1 mm or less (i.e., Control B's LII reduced to 0.7 mm). The rate of Control B's tooth movement during the study period was only about 0.39 mm/week.

It is noted that one or more patients may have achieved an LII value of 1 mm or less before the patient progressed to using the 16×16 nickel titanium wire.

### Example 9

The results of the Intra-Oral Group of Example 3 were compared to the results of an Extra-Oral Group and a Control Group. Specifically, data regarding the rate of tooth movement (also referred to as “alignment rate”) for the Intra-Oral Group were compared to the rate of tooth movement for the Control Group, which included eight (8) patients who also wore conventional buccal fixed orthodontic brackets, i.e., one of SPEED System™ brackets or Agility® self-ligating brackets (commercially available from Orthodontic Design and Production, Inc.), and wires, as described in Example 3 herein, during the alignment phase of his or her orthodontic treatment, but did not receive light therapy. Two of the eight Control Group patients were Controls A and B of Example 8. The Control Group is a subset of a control group of patients from a multi-site, multi-investigator study. The pooled control group patients had a mean rate of tooth movement of about 0.50 mm per week. In comparison, the mean rate of tooth movement during the study period for Patients D-L was about 1.32 mm per week, which is about 2.64 times greater than expected based on the rate of tooth movement of the pooled control group patient data. The eight Control Group patients had a mean rate of tooth movement of 0.32 mm per week. In comparison, the mean rate of tooth movement during the study period for the Intra-Oral Group was about 3.6 times greater than expected based on the rate of tooth movement of the Control Group patient data. Additionally, while the mean time to alignment for the Intra-Oral Group was 41 days, the Control Group had a mean time to alignment of 115 days, thus demonstrating significant improvement with the intra-oral apparatus.

Data regarding the rate of tooth movement for the Intra-Oral Group and the Control Group were also compared to the rate of tooth movement for the Extra-Oral Group, which included thirteen (13) patients receiving extra-oral light therapy in combination with orthodontic treatment.

Specifically, in the Extra-Oral Group, each of the thirteen patients used an extra-oral apparatus (depicted in FIG. 21A of International Patent Publication No. WO 2012/0075584, which is incorporated by reference herein in its entirety, and reproduced here as FIG. 83) for light treatment and wore conventional fixed brackets. i.e., one of SPEED System™ brackets or Agility® self-ligating brackets that were aligned with 0.014 or 0.016 inch nickel titanium wire and final rectangular alignment wire of at least 0.017 inch by 0.025 inch (also referred to as “17×25”) nickel titanium wire. The extra-oral apparatus used by each patient included a set of four extra-oral light arrays, each of which includes a flexible printed circuit board and a set of LEDs. The light arrays were positioned extra-orally in contact with each patient's face. The light arrays were coupled to a handheld controller via conductive cables. A headset of the apparatus included attachment and adjustment mechanisms for positioning on the patient's face such that the light arrays were directed to a target oral tissue.

Light was administered to the Extra-Oral Group using the extra-oral apparatus which irradiated light at a near infrared wavelength of about 850 nm (±5 nm) and an intensity of ranging from about 60 mW/cm² to about 100 mW/cm². The mean intensity of the irradiated light for the Extra-Oral Group was about 70 mW/cm². Patients in the Extra-Oral Group were randomized into one of three groups receiving light treatment of 20 minutes/day, 30 minutes/day, or 1 hour/week, respectively. During the study, patients in the Extra-oral Group and the Control Group) visited a clinician every two weeks for a six week period from the beginning of the study period and then every four weeks until alignment (as defined in Example 3 herein) was achieved. At each visit, the clinician performed regular orthodontic procedures, collected data that included photographs representing the occlusal and buccal views of the patient's dentition and recorded patient compliance, which was monitored by a microprocessor in the controller of the extra-oral apparatus. The effectiveness of the treatment was assessed using the LII grading system, in a manner similar to that described in Example 3.

It should be noted that the patients in the Extra-Oral Group and the Control Group are a subset of a larger group of patients of a multi-site and multi-investigator study; however, the subset was selected for comparison with the Intra-Oral Group because its patients were from the same single site and assigned to the same investigator as the patients described in Example 8.

In total, the comparison of data from the Intra-Oral Group, the Extra-Oral Group and the Control Group included data for twenty-three (23) maxillary arches and seven (7) mandibular arches. Of the thirty (30) total arches, eighteen (18) are arches of female patients and twelve (12) are arches of male patients, and twenty-three (23) are arches of Caucasian patients and seven (7) are arches of non-Caucasian patients of various other ethnicities. The patients ranged in age from 11 years old to 27 years old, with a mean age of 14 years. Each patient had an initial LII (i.e., on day 1 of the study period for each patient) ranging from 3 mm to 12 mm.

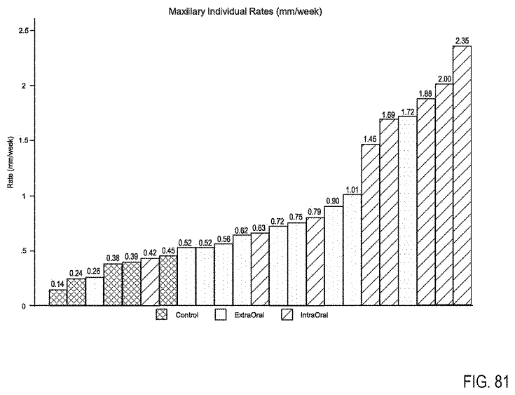

The rate of individual patient's maxillary tooth movement is represented in the bar graph in FIG. 81, in which individual patients are shown on the x-axis and the rate of tooth movement in mm per week is shown on the y-axis. As illustrated, the individual rates of maxillary tooth movement for the Intra-Oral Group ranged from 0.42 mm per week to 2.35 mm per week, for the Extra-Oral Group ranged from 0.26 mm per week to 1.72 mm per week, and for the Control Group ranged from 0.14 mm per week to 0.45 mm per week.

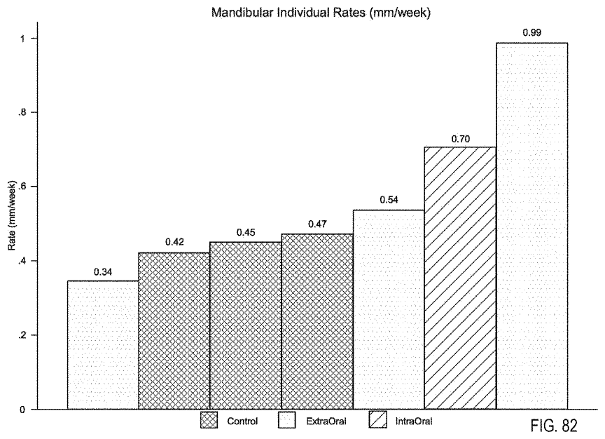

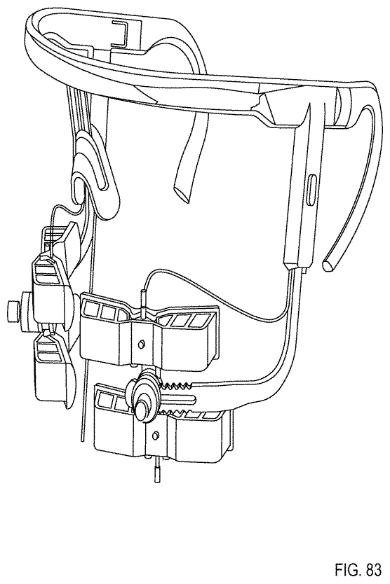

The rate of individual patient's mandibular tooth movement is represented in the bar graph shown in FIG. 82, in which individual patients are shown on the x-axis and the rate of tooth movement in mm per week is shown on the y-axis. As illustrated, the individual rate of mandibular tooth movement for the Intra-Oral Group was 0.79 mm, for the Extra-Oral Group ranged from 0.34 mm per week to 0.99 mm per week, and for the Control Group ranged from 0.42 mm per week to 0.47 mm per week.

As noted above, the mean rate of tooth movement during the study period for the Intra-Oral Group was about 1.32 mm per week. The Extra-Oral Group had a mean rate of tooth movement of about 0.73 mm per week, with a standard deviation of 0.37 mm. The Control Group had a mean rate of tooth movement of about 0.37 mm per week, with a standard deviation of 0.12 mm.

Analysis of the data in the comparative study showed that the alignment rate results achieved in the Intra-Oral Group, i.e., resulting from the patient's combined usage of the intra-oral light therapy apparatus to administer light therapy and conventional orthodontic brackets, are statistically significant (at p<0.01) when compared to the alignment rate results of the Control Group, in which no patient received light therapy during the study period. Analysis of the data in the comparative study showed that the alignment results achieved in the Extra-Oral Group, i.e., resulting from the patient's combined usage of the extra-oral apparatus to administer light therapy and conventional orthodontic brackets, are statistically significant (at p<0.1), albeit less so than in the Intra-Oral Group, when compared to the alignment rate results of the Control Group, in which no patient received light therapy during the study period.

### Example 10

In one study, a mandibular anterior intra-oral light therapy apparatus of the invention, depicted in FIG. 44, was used during a post-operative period to irradiate the root area of the lower jaw of a patient. The intra-oral light therapy apparatus included an intra-oral housing with LEDs mounted on flexible printed circuit boards, which were disposed in an interior region of soft flexible buccal flanges of the intra-oral housing. The interior region of the buccal flanges is formed between opposing outer surfaces, in which the first outer surface is disposed on the buccal side of the flange and the second outer surface is disposed on the lingual or palatial side of the flange. Specifically, during the manufacturing process, the printed circuit boards were inserted into the interior region of each of left side and a right side buccal flange from the bottom of the buccal flanges, and then the bottoms of the buccal flanges were coupled using adhesive to a bite plane portion, thereby scaling the LEDs mounted on the flexible printed circuit boards within the buccal flanges.

On day 1 of the study, six (6) dental implants were placed in the anterior portion of the patient's lower jaw. On day 2 of the study, a temporary crown was loaded on each of the six implants. The intra-oral light therapy apparatus was used by the patient every day for seven (7) days post-operatively, beginning on day 1, and on that day, within about 1 hour of placement of the dental implants. When in use, the intra-oral light therapy apparatus irradiated the root tissue of the lower jaw with light having a wavelength of about 850 nm (±5 nm). Light therapy was administered to the patient's root tissue of the lower jaw for about six minutes per day, and at a light output intensity of about 100 mW/cm².

An Osstell™ Mentor device (commercially available from Osstell AB) was used to measure stability of the implants at the time of surgery (i.e., on day 1), and again on day 7 to measure any relative change in the implants' stability. The Mentor device uses resonance frequency analysis to measure stability, presented as an implant stability quotient (ISQ). The stability measured on day 7 showed no net decrease in stability of the six implants from their stability measured on day 1. More specifically, the average ISQ value measured on day 7 was unchanged from the average ISQ value measured on day 1. Such a result stands in stark contrast to the typical decrease in stability of dental implants that follows their placement and that can occur for up to 90 days thereafter, before the implants regain stability comparable to that of the implants on the day of their placement. Without being bound by theory, the typical decrease in stability is believed to be due to inflammatory resorption of bone supporting the dental implants and believed to result from the surgical process of drilling into the bone. It is noted that the patient had a history of very poor post-operative soft-tissue healing, including unwanted exposure of underlying bone, from previous implant surgeries on the right and left posterior quadrants of the patient's lower jaw. In this case, however, the patient's soft tissue healing at day 7 was optimal compared to what the patient experienced from the previous implant surgeries with the two lower posterior dental quadrants.

### Example 11

In one study, an illustrative intra-oral light-therapy apparatus of the invention was used during the space closure phase of orthodontic treatment to irradiate the maxillary and/or mandibular arches of twenty-three (23) patients with a total of sixty-seven (67) extraction quadrants (the “Intra-Oral Group”). The intra-oral light-therapy apparatus included an intra-oral housing including a flexible circuit of light arrays of LED light emitters embedded in medical-grade silicone buccal flanges of the intra-oral housing, similar to the intra-oral housing shown in FIG. 94. The intra-oral housing was electronically coupled via a connector to a hand-held controller disposed external to the intra-oral housing. The controller included a microprocessor, an LCD screen and processor-executable code. The controller coupled the intra-oral housing with a power source. Each patient was provided with his or her own intra-oral light-therapy apparatus, which was used in combination with a conventional fixed orthodontic appliance, as described herein. The treatment and results of the patients in the Intra-Oral Group were compared to the treatment and results of patients in a Control Group, which included twenty-two (22) patients with fifty-six (56) extraction quadrants who wore conventional fixed appliances, as described herein, during the space closure phase of his or her orthodontic treatment, but did not receive light therapy. The treatment and results of the patients from the Intra-Oral Group and Control Group are described in detail herein.

During the study and prior to the start of treatment, each patient was provided full mouth scaling and polishing and was given appropriate oral hygiene instructions. Routine orthodontic records including study casts, photos, a panoramic radiograph (OPG) and lateral cephalogram were taken. When each patient reached the active space closure phase of his or her orthodontic treatment, impressions were taken to construct new models (T1).

All patients were treated with conventional fixed appliances with a slot size of 0.022×0.028, MBT prescription. Bonding methods were standardized in all patients using bonding adhesive composite Transbond XT (available from 3M Unitek). Extraction spaces were closed by sliding mechanics with the posterior anchorage segment held together by FIG. 8 steel ligature to ensure a single anchorage unit. Extraction spaces were closed using nitinol (NiTi) closed-coil springs (available from G&H Wire Co.) from the hook of the first molar to the hook of the working wire (19×25 stainless steel). One hundred and fifty (150) grams of force was applied throughout the space closure duration. This level of force was maintained, regardless of the amount of extraction spaces, by using a dynamometer and manufacturer guidelines. Active space closure was done on 0.019×0.025 inch stainless steel wires with hooks. Miniscrews were utilized for space closure in some patients, however randomization was carried out that such that the same number of patients in which miniscrews were utilized were in each group.

During the study, the intra-oral light-therapy apparatus was used by each patient in the Intra-Oral Group every day until space closure was achieved. When in use, the intra-oral light-therapy apparatus irradiated the patient's oral tissue with light having a wavelength of about 850 nm and a power output of about 33 mW/cm² for about three minutes per day per treated arch to yield a gum-line dose of about 6 J/cm². The microprocessor of the controller tracked usage of the intra-oral light therapy apparatus. Patients were expected to maintain 75% compliance or greater throughout the duration of space closure. If one or two sessions were missed, patients were advised to make up for the few lost sessions spread throughout the following day.

During the study, the patients visited a clinician every two (2) weeks for the first three visits, and then every two (2) to four (4) weeks based on the patient's compliance level and any complication regarding orthodontic appliance breakage. At each visit, the clinician performed regular space closure recall procedures, retrieved patient compliance data, checked the functionality of the intra-oral light-therapy apparatus, and visually inspected the patient for space closure. Active spaced closure was considered complete (T2) when the space that remained was 2 mm or less, as determined clinically by visual inspection, or when molar anchorage was lost in the judgment of the clinician. When T2 was reached, impressions were taken of each patient to construct a T2 model, intra-oral photographs were taken, the light therapy apparatus was returned to the clinician, and the patient was exited from the study.

The amount of extraction spaces before active space closure was measured on the T1 models (minimum of 3 mm) using a digital caliper with accuracy up to 0.01. Reference points were the free gingival margin of each end of the extraction space. Spaces present mesial to the patient's canine tooth (or teeth) were measured and added to the extraction space to be closed on both sides. Each measurement was taken by an examiner who was blind as to whether the model was for a patient in the Intra-Oral Group or Control Group. Where possible, the examiner was also blind as to whether the model was a T1 model or a T2 model. The study models were measured randomly so that T1 and T2 models of the same patient were not measured consecutively. Measurements were taken twice, at spaced intervals, for each model, and the average of the two measurements was used as the final value.

Patients were selected for participation in this study based on, at least, the following criteria: (1) a presence of permanent dentition (i.e., permanent, or adult, teeth); (2) the patient was engaged in comprehensive orthodontic therapy, which therapy included an extraction treatment plan for extraction of the patient's first or second premolar in at least one of the upper arch or lower arch, or both arches; (3) the patient had a minimum of a 3 mm extraction space to be closed in at least one quadrant of the patient's upper and/or lower arches; (4) the patient demonstrated good oral hygiene; (5) the patient was not undergoing adjunct orthodontic treatment, such as that involving extra- or intra-oral appliances; and (6) the patient was from 12 to 40 years old. The following types of individuals were excluded from this study: (1) individuals with primary or mixed dentition; (2) individuals with active periodontal disease or evidence of horizontal bone loss; (3) individuals who could not maintain a minimum compliance rate of 75% in using the intraoral light therapy apparatus; or (4) individuals with any systemic disease or using any medication that may affect bone metabolism, such as long term use of NSAIDs or bisphosphonates, among others.

Patients in the Intra-Oral Group had space closure rates in the range of about 0.41 mm/month to about 2.80 mm/month, with a standard deviation of about 0.49 mm/month. The Intra-Oral Group had a mean space closure rate of 1.07 mm per month. Patients in the Control Group had space closure rates in the range from about 0.12 mm/month to about 1.79 mm/month, with a standard deviation of 0.37 mm/month. Individual patient data for each of the Control and Intra-Oral Groups is shown in FIGS. 144A, 144B, 145A and 145B, respectively. In FIGS. 144A-B and 145A-B, data for each patient in the study includes the tooth number extracted, the total extraction space in the T1 model in millimeters, the total extraction space in the T2 model in millimeters, and the time in days to achieve space closure for the identified tooth number and patient. The tooth numbering shown in FIGS. 144A-B and 145A-B is based on the ISO and FDI World Dental Federation numbering systems. The Control Group had a mean space closure rate of 0.85 mm per month. The mean space closure rate of the Intra-Oral Group is statistically significantly faster (p-value<0.05, i.e., 0.0047) than the mean space closure rate of the Control Group.

The results regarding the patient's velocity of space closure were also analyzed by age per group. Specifically, within each group, the space closure rate data was separated by youth (11-19 years old) and adults (20-42 years old). Youth patients in the Intra-Oral Group had space closure rates in the range of 0.53 mm/month to 2.80 mm/month, with a standard deviation of 0.49 mm/month. Youth patients in the Control Group had space closure rates in the range of 0.44 mm/month to 1.79 mm/month, with a standard deviation of 0.36 mm/month. The mean space closure rate of the youth patients in the Control Group was 0.93 mm/month, and the mean space closure rate of the youth patients in the Intra-Oral Group was 1.20 mm/month. The mean space closure rate of the youth patients in the Intra-Oral Group is statistically significantly faster (p-value<0.05, i.e., 0.0068) than the mean space closure rate of youth patients in the Control Group. The youth patients in the Intra-Oral Group demonstrated a 28% improvement in the rate of space closure when compared to the youth patients in the Control Group.

Adult patients in the Intra-Oral Group had space closure rates in the range of 0.56 mm/month to 1.67 mm/month. Adult patients in the Control Group had space closure rates in the range of 0.12 mm/month to 0.87 mm/month. The mean space closure rate of the adult patients in the Control Group was 0.52 mm/month, and the mean space closure rate of the adult patients in the Intra-Oral Group was 0.77 mm/month. The mean space closure rate of the adult patients in the Intra-Oral Group is statistically significantly faster (p-value<0.05, i.e., 0.0426) than the mean space closure rate of adult patients in the Control Group. The adult patients in the Intra-Oral Group demonstrated a 48% improvement in the rate of space closure when compared to the adult patients in the Control Group.

In conclusion, the results of the study demonstrate that use of light therapy in combination with conventional orthodontic treatment is statistically significantly effective at accelerating the rate of space closure for extraction cases in both the maxilla and mandible arches in both the adult and youth population.

### Example 12

This example provides results from a prospective, crossover, single center pilot study aimed at observing the clinical efficacy of a light therapy apparatus described herein or of AcceleDent® on the rate of patient progression through INVISALIGN™ (Align Technology, Inc.) aligners and thus, the rate of tooth movement during aligner treatment.

By utilizing a crossover design, the patient becomes his or her own control, allowing comparison of the patient's natural rate of tooth movement and aligner switching with the patient's rate of aligner switching when using an interventional light therapy apparatus. In this study, interventions are conducted with a light therapy apparatus described herein or an AcceleDent device. When comparing the aligner switching rate for a patient during the non-intervention (control) period with the aligner switching rate during the intervention period (crossover design), the effect of the intervention apparatus on the rate of switching aligners can be determined.

INVISALIGN™ aligners are designed to move the teeth by a predetermined fixed amount per aligner, and their manufacturer instructs replacing these aligners on a fixed schedule, typically every 7 or 14 days. However, individual biological variation contributes to the rate at which teeth move—some patients' teeth move fast, while other patients' teeth move slower. Accordingly, each patient has his or her own unique rate of tooth movement, which translates into a unique rate of when aligners should be switched.

This example provides a method for determining a customized rate of aligner switching for each individual patient. The study utilized a daily self-evaluation for aligner fit, pressure and pain, which allows patients to switch aligners based on their natural tooth movement rate.

Inclusion:
Presence of permanent dentition
Eligible and scheduled for full mouth fixed orthodontic treatment.
Good oral hygiene, as determined by the Qualified Investigator orthodontist.
Age 12-60
Non-smoker

Exclusion:
Pregnant females
Patients currently enrolled in another clinical study
Non-steroidal Anti-Inflammatory drug (NSAID) use during study (Acetominophen acceptable)
Periodontally involved teeth
Use of bisphosphonates

The two INVISALIGN™ study groups were as follows:

Study Group 1: Patients were prescribed a series of conventional INVISALIGN™ aligners, each of which is programmed to laterally translate a tooth ¼ mm during a 14 day period, at which point it is replaced.

Study Group 2: Patients were prescribed a series of conventional INVISALIGN™ aligners, each of which is programmed to laterally translate a tooth ⅛ mm during a 7 day period, at which point it is replaced. Accordingly, the “⅛ mm” aligner is the same aligner as the “¼ mm” aligner, but is prescribed to be replaced every 7 days instead of every 14 days. A patient in the ¼ mm group uses half as many aligners as a patient in the ⅛ mm study group, if both patients cycle through aligners for the same amount of total time and both patients follow the orthodontist's prescription to replace the aligners every 7 days or every 14 days, respectively. The aligner replacement is made by the patient and assumes his or her compliance with an orthodontist's instructions.

The 3 Intervention Groups were as follows:

Intervention Group 1 (OPx1): light therapy apparatus of FIG. 132, (ORTHOPULSE™) 5 min/arch=10 min daily.

Intervention Group 2 (OPx2): light therapy apparatus of FIG. 132, (ORTHOPULSE™) 10 min/arch=20 min daily.

Intervention Group 3 (AD): AcceleDent 20 min daily, both arches.

Each patient served as his or her own control by progressing through aligners with no intervention, establishing a control period. Patients progressed through at least 6 aligners in each control period (±1 aligner). During the control period, the patient self-assessed aligner fit on a daily basis, based on pressure, pain, and number of air bubbles present (see below regarding method of self-assessment). This established a baseline aligner-change interval for each individual patient. After the control period, an intervention apparatus was used on a daily basis while the patient continued progressing through aligners, establishing an intervention period (see above for the three intervention groups). Patients progressed through at least 6 aligners in each intervention period (±1 aligner). After a light therapy apparatus patient completed a first invention period (e.g., either OPx1 or OPx2), the patient was given the opportunity to complete a subsequent light therapy intervention period. The light therapy patient was given the option regarding the intervention treatment protocol (i.e., either OPx1 or OPx2) for the subsequent intervention period, although OPx1 patients were recommended to complete an OPx2 intervention period as the subsequent period. Similarly, OPx2 patients were recommended to complete an OPx1 intervention period as the subsequent period. The baseline aligner change interval for each individual patient (control period) was compared to his or her aligner change interval during intervention. Aligners were changed by a method of self-assessment, as described below.

Each patient progressed through each aligner by a method of self-assessment, via an online daily questionnaire. Every day, the patients assessed:

(A) pain levels: 1 (no pain or almost no pain), 2 (some pain), 3 (much pain); (B) aligner fit/pressure on teeth: 1 (no pressure or almost no pressure), 2 (moderate pressure), 3 (high level of pressure); (C) air gaps present between aligner and teeth: rated 1-3, where 1 indicates no air gaps. Each patient progressed into the subsequent aligner when pain score was 1, aligner fit score was land no air gaps were present.

The average number of days that aligners were used during the control (no intervention) period was compared to the average number of days that aligners were used during the intervention period. The percent reduction of the average number of days that aligners were used was compared to conventional INVISALIGN™ aligner change frequency for each of the control, OPx1, OPx2 and AcceleDent periods.

Results

Thirteen patients have completed a control period and at least one intervention period (6 patients were treated with ¼ mm INVISALIGN™ aligners and 7 patients were treated with ⅛ mm INVISALIGN™ aligners).

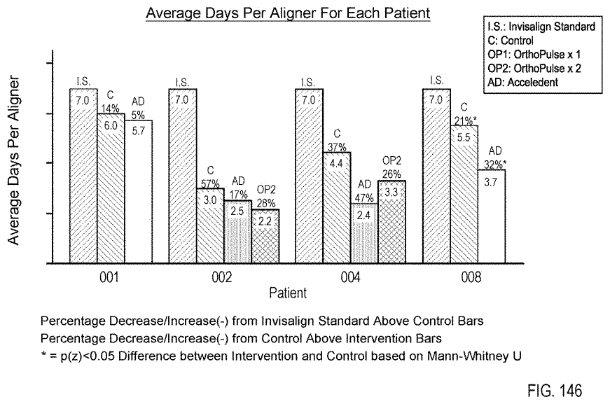

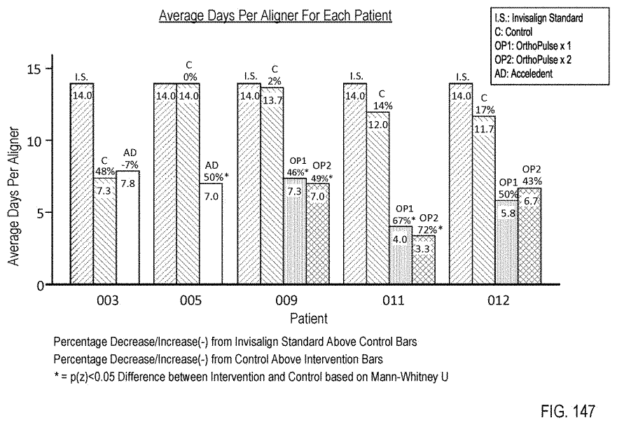

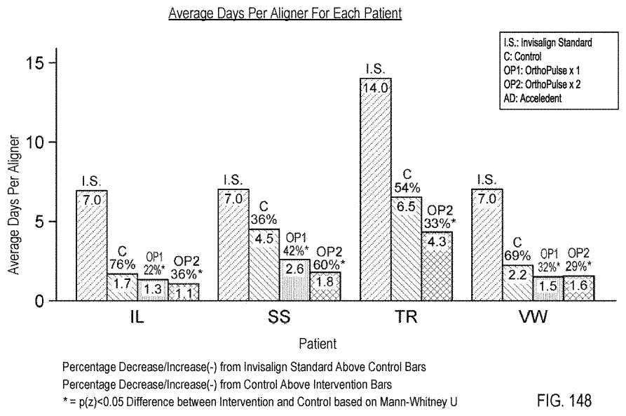

The results of the study are provided in FIGS. 146-148. In each of these figures, the percent value above each control bar represents improvement over conventional INVISALIGN™ switching frequency. The percent value above each intervention bar (OP1, OP2, AD) represents improvement over the same patient's control period. Mann-Whitney U test results are reported above intervention bars comparing intervention to control within each patient (*=p<0.05).

Days per aligners for all AcceleDent patients were compared to their control, and light therapy patients were also compared to their own control for each individual patient (FIGS. 146-148). Percentage reductions of aligner switching rate for each of the INVISALIGN™ study groups (i.e., (i) ¼ mm group and (ii) ⅛ mm aligner group) were pooled, and are presented in Table 1. Two-tailed T-tests were used to compare the significance levels of comparisons.

TABLE 1
Percent reduction for pooled ¼ mm and ⅛ mm INVISALIGN ™ aligner.

| | | | Percentage Reductions (%) | | | | | |
|---|---|---|---|---|---|---|---|---|
| | Patient | Aligner | From | p- | From | p- | From | p- |
| Intervention | N | N | prescribed | value | control* | value | AcceleDent** | value |
| Control (AD) | 4 | 19 | 21% | 0.00 | — | — | — | — |
| AcceleDent | 4 | 41 | 43% | 0.00 | 17 | 0.00 | — | — |
| Control (OP) | 9 | 46 | 46% | 0.00 | — | — | — | — |
| ORTHOPULSE ™ | 8 | 70 | 66% | 0.00 | 51% | 0.00 | 41% | 0.00 |
| OPx1 | | | | | | | | |
| ORTHOPULSE ™ | 9 | 107 | 70% | 0.00 | 56% | 0.00 | 48% | 0.00 |
| OPx2 | | | | | | | | |

*two-tailed t-tests used for comparing within patient aligner spells, i.e., the duration of time for each individual aligner (i.e., either ⅛ or ¼ mm aligner) compared to Control
**two-tailed t-tests used for comparing full pooled aligner spell groups (i.e., the duration of time for patients treated with ⅛ and ¼ mm aligners, pooled, not just within patient groups) to AcceleDent

The results indicate that by using patient self-evaluation of aligner fit and pressure, each patient's natural rate of switching aligners was able to be determined. ORTHOPULSE OPx1 and OPx2 treatments resulted in a significant reduction in the number of days required per aligner as compared to each patient's own control: 51% reduction for OPx1 and 56% reduction for OPx2. A greater reduction in number of days required per aligner was observed for OPx1 and OPx2 patients, as compared to the AcceleDent patients.

### Example 13

Study Overview and Aims

This crossover study demonstrates that daily ORTHOPULSE™ (see, e.g., the apparatus set forth in FIGS. 126 and 127) use increases the rate of orthodontic tooth movement during the alignment phase of orthodontic treatment of patients using INVISALIGN™ aligners on their mandibular arch. The study also demonstrates that patients treated with ORTHOPULSE™ do not experience root resorption beyond what is usually expected during orthodontic treatment. The study further demonstrates that patients treated with ORTHOPULSE™ do not experience gingival recession or tooth instability.

The study also demonstrates the safety of the apparatus.

A total of 21 patients (from 14 to 53 years old), suitable for INVISALIGN™ treatment, in a private practice in Chandler, Ariz., were enrolled in this study. The Principal Investigator (PI) assessed every patient that was interested in the study for eligibility based on the inclusion/exclusion criteria. The study design and all patient forms, including the study informed consent/assent forms, received ethics approval by IRB Services. Every patient was required to review and sign the informed consent/assent forms prior to being enrolled in the study.

By implementing a crossover design and aligner change protocol, patients established their own baseline rate of tooth movement through a set of 4 aligners (no ORTHOPULSE™ treatments). The ORTHOPULSE™ rate of tooth movement was determined by introducing daily ORTHOPULSE™ treatments as the patients progressed through another set of 4 aligners. ORTHOPULSE™ is a near-infrared light treatment apparatus that irradiates at a continuous 850-nm wavelength. Patients were randomly assigned to either Group A or Group B. Both Groups started with a 2-aligner initiation period. Group A began a 4-aligner period of carrying out 5-minute daily ORTHOPULSE™ treatments per arch. Group B progressed through these same aligners without ORTHOPULSE™ treatment. Both Groups performed a single-aligner washout period and then carried out a 4-aligner period without and with daily ORTHOPULSE™ treatments for Group A and B, respectively.

Perimeter measurement analysis was used to evaluate each patient's rate of tooth movement during baseline and ORTHOPULSE™ periods in their mandibular arch. A comparison of the rate of change between baseline and ORTHOPULSE™ for each patient allowed for a direct measure of the effect of ORTHOPULSE™ treatment on tooth movement during resolution of anterior crowding.

The severity of external apical root resorption was also investigated in order to determine whether resorption occurred beyond what is typical of orthodontic treatment. Root resorption was determined using panoramic dental x-rays collected before treatment and after 6-months of INVISALIGN™ treatment.

Study Population

A total of 21 patients from 14 to 53 years old received INVISALIGN™ treatment in combination with 5-minute daily ORTHOPULSE™ treatments (OP), per arch.

A total of 17 patients reached the primary outcome of the study, providing complete baseline and ORTHOPULSE™ tooth movement data. The average tooth movement rates were 0.126 and 0.231 mm/week for the baseline and ORTHOPULSE™ periods, respectively. This indicates that the rate of tooth movement for the ORTHOPULSE™ period was 1.8-fold faster than that of the baseline period, with significance (p-value=0.02). The presence of period effects were not supported. Carryover effects were also undetected, likely due to the adequate washout period utilized in this study.

The overall mean External Apical Root Resorption (EARR) was −0.673 mm, indicating marginal root elongation rather than resorption. Thus, no mean root resorption was detected after 6 months of ORTHOPULSE™ treatment. There was no gingival recession or tooth instability reported by the PI at any point during the course of the study.

There were no patients discontinued from the study due to negative adverse events. There were no negative adverse events or side effects reported in this study, and none of the patients reported using anything beyond over-the-counter (OTC) medication to alleviate tooth and mouth discomfort.

In summary. ORTHOPULSE™ is useful for increasing the rate of orthodontic tooth movement, particularly during the alignment phase of orthodontic treatment, and decreasing alignment-treatment time.

For this study, ethical approval was obtained from the Institutional Review Board (IRB) Services, ON, Canada, which approvals sites in both Canada and the United States. The study design and all patient forms, including the informed consent form, received ethics approval by IRB Services.

Apparatus Description

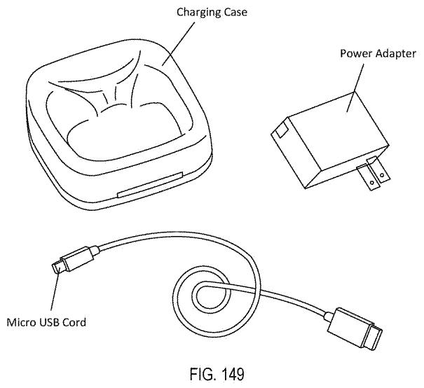

Test subjects received daily treatment with an intra-oral apparatus. ORTHOPULSE™ (Biolux Research, Vancouver, Canada), which produces near-infrared light with a continuous 850-nm wavelength and is depicted in FIG. 126 and FIG. 127. The power density of the ORTHOPULSE™ LED array is 63 mW/cm² (+/−13 mW/cm²). The ORTHOPULSE™ apparatus was used for 5 minutes per arch per day to achieve a total energy density of approximately 19.5 J/cm² at the surface of the array.
The ORTHOPULSE™ package consists of five components (FIG. 149):
Mouthpiece
Charging Case
Power Adaptor
Micro USB Cord
ORTHOPULSE™ Manual

ORTHOPULSE™ is a wireless, battery-powered, intraoral apparatus made of medical grade silicone embedded with flex circuit arrays of LED emitters. Light is generated inside the mouth and delivered through the alveolar soft tissue into the alveolar mucosa. The apparatus keeps a record of usage, which can then be interfaced with a mobile device application to log treatment compliance data. ORTHOPULSE™ is approved for sale in Canada, is considered a Class II Medical Device in the US and is a CE Mark approved Class 2a Device in Europe. For the purpose of this study, use of ORTHOPULSE™ in the protocol was determined to be a non-significant risk.

Crossover Design

By implementing a crossover design and aligner change protocol, patients established their own baseline rate of tooth movement through a set of 4 aligners (no ORTHOPULSE™ treatments). The ORTHOPULSE™ rate of tooth movement was determined by introducing daily ORTHOPULSE™ treatments as they progressed through another set of 4 aligners. ORTHOPULSE™ is a near-infrared light treatment apparatus with a continuous 850-nm wavelength. Patients were randomly assigned to either Group A or Group B. Both Groups started with a 2-aligner initiation period. Group A began a 4-aligner period of carrying out 5-minute daily ORTHOPULSE™ treatments per arch. Group B progressed through these same aligners without ORTHOPULSE™ treatment. Both Groups performed a single-aligner washout period and then carried out a 4-aligner period without and with daily ORTHOPULSE™ treatments for Group A and B, respectively.

Anterior tooth alignment was measured using an arch perimeter analysis, as described by Howe et al., Am J Orthod. 1983 May; 83(5):363-73) and used to quantify crowding. There is an expansion component and alignment component applied when crowded anterior teeth are treated with a series of aligners. Both components are the result of tooth movement. The arch perimeter analysis aims to measure the perimeter of the arch prior to expansion and alignment. When the arch perimeter is also measured after a period of time, the amount of expansion and tooth movement during that period of time can be determined. Moreover, the rate can be calculated when the amount of time between the two measurements points is factored into the change in the perimeter.

Study Objectives

This study was a prospective, interventional, crossover, open label, randomized, single-center study designed to evaluate the intended purposes of the investigation. The primary objective of this study was to determine whether daily ORTHOPULSE™ use affects the rate of orthodontic tooth movement during the alignment phase of orthodontic treatment in patients using INVISALIGN™ aligners.

The secondary objectives of this study were:
To collect confirmatory evidence on the safety of the apparatus.
To determine whether the apparatus had an effect on the amount of external apical root resorption (EARR) no earlier than 6 months after starting orthodontic treatment with INVISALIGN™ aligners.

Null Hypothesis
There is no difference in the rate of orthodontic tooth movement during alignment with INVISALIGN™ aligners utilizing the arch perimeter analysis method.
There is no difference in safety during ORTHOPULSE™ use and non-ORTHOPULSE™ use.
ORTHOPULSE™ has no effect on the amount of external apical root resorption (EAER) no earlier than 6 months after starting orthodontic treatment with INVISALIGN™ aligners.

Statistical Determination of Sample Size

A sample size calculation was performed using levels of significance and statistical power consistent with the objective of the study. The parameters used in the sample size calculation were based on conservative assumptions and findings from previous studies. The derived sample size represented an estimated value considered likely to produce statistically significant results. The sample size calculation was conducted using the Statistical Software Stata 12 (StataCorp, College Station, Tex., USA) under the following assumptions:
Outcome Variable: Rate of tooth movement rate in mm/week per perimeter analysis.
Outcome Variable Mean Value: Based on percent improvement found in the pilot study as well as the results found in other studies expecting a 50% improvement is a very conservative estimate for cases treated with intra-oral light therapy.
Outcome Variable Distribution: Normal distribution (Gaussian) with a standard deviation □ equal to the Outcome Variable Mean Value. This is a conservative assumption since in the current study treated patients serve also as their own controls, removing biological variations between patients.
Minimum Effect Clinically Important: δ=50%
Type I Error (α): the probability of erroneously rejecting the null hypothesis I=5% (i.e., α=0.05). This value is consistent with the approach commonly used in cases where the control treatment is widely used and is known to be reasonably safe and effective.
Type II Error (β): the probability of erroneously failing to reject the null hypothesis I=20% (i.e., β=0.2). Therefore, the Statistical Power (1−β)=80%. This is consistent with the approach commonly used in preliminary trials that are likely to be replicated.

Assuming a mean difference of 50% with a standard deviation equal to that of the mean and two-tailed α of 0.05, a group of 10 patients, each with crowding resolution rates under treatment and baseline will provide power of 0.80.

To satisfy these sample size requirements, account for possible patient withdrawals, and achieve a greater power, the final enrolment of this study was 21 patients.

Sample Selection

The sample consisted of patients who were eligible and scheduled for INVISALIGN™ treatment at a private practice office in Chandler, Ariz. Patients were recruited for the study given the following inclusion and exclusion criteria:

Patient Inclusion Criteria
Permanent dentition
Eligible and scheduled for INVISALIGN™ treatment
Mild to moderate crowding, with no spaces between anterior teeth
Class I or Class II by ½ cusp or less
Age 12-60 years
Good oral hygiene
Non-smoker

Patient Exclusion Criteria
Pregnant females
Patient is currently enrolled in another clinical study
Periodontally involved teeth
Use of bisphosphonates (osteoporosis drugs) during the study
Patient plans to move over the treatment period

Randomization

The Principal Investigator (PI) assessed each patient interested in the study for eligibility, based on the inclusion/exclusion criteria outlined above. If all selection criteria were met, subjects were asked to participate in this study.

All subjects were required to sign the IRB ethics approved informed consent.

Once subjects were enrolled in the study, they were assigned to either Group A or Group B according to a permuted-block randomization created by a statistician. The Group A versus Group B roster was collated with the block-randomized list, blocks of 4 and 6, to generate a list of patient IDs with randomized group assignment. This list was generated prior to the start of the study and delivered to the practitioner. The practitioner assigned patient IDs in chronological order of enrolment.

Patients were prescribed 14-day aligners for the entirety of their INVISALIGN™ treatment.

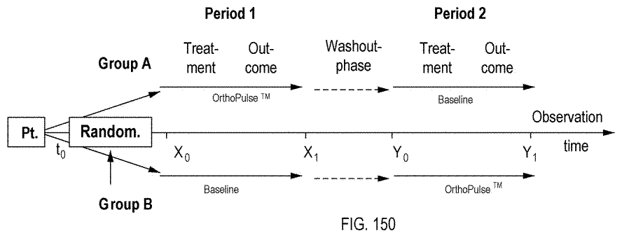

The design and structure of the Crossover Trial and Orthodontic Procedures used therein is set forth in FIG. 150. The two phases that each patient has to complete in the course of this study are referred to as test periods, each spanning the duration of 4 aligners. The first test period is defined as period X and second defined as period Y. X0 and X1 define the start and end of the period X, respectively. Y0 and Y1 define the start and end of the period Y, respectively.

Each patient was assigned to Group A or Group B in random order, as disclosed herein. Group A patients had daily ORTHOPULSE™ treatments during the first test period and no ORTHOPULSE™ treatments during the second test period. Conversely, Group B patients established their baseline during the first test period and had daily ORTHOPULSE™ treatments during second test period.

The efficacy of treatment periods X and Y was assessed on the basis of within-subject difference between the two treatments with regard to the outcome variable.

The INVISALIGN™ treatment process involved scanning all patients using the iTero® digital scanner by Cadent, a subsidiary of Align Technology. Using the PI's treatment goals, INVISALIGN™ technicians created a treatment plan. Once the PI approved the plan, the computer model created stages between the current and desired tooth positions that were used to create a series of INVISALIGN™ aligners. The PI also captured pre-treatment panoramic dental radiographs at this time.

During the first appointment, ORTHOPULSE™ was demonstrated and delivered to each patient. Patients were taught how to use, clean, store, and maintain the apparatus, and were informed that they were expected to maintain at least 80% compliance (6 out of 7 days) to daily ORTHOPULSE™ use during their ORTHOPULSE™ treatment period.

The patients had a 2-aligner initiation period, of 14-days in each aligner, to become familiar with using aligners. After this initiation period, the PI prescribed to each individual patient that they change aligners based on specific criteria. Subsequent to the initiation period, patients underwent two test periods, X and then Y with 4-aligners in each period. Daily ORTHOPULSE™ treatment was administered according to whether the patient was in Group A or Group B patient (see above). One-aligner washouts periods occurred between the two test periods, and patients received a follow-up visit after each test period (see FIG. 150).

Patients completed a brief daily questionnaire, which required a self-assessment of the level of pain, aligner pressure, and presence of air gaps between their teeth and the aligner. All questions were rated on a scale between 1 and 3, with 3 being the highest perceived level of pain pressure or presence of air gaps. Patients were instructed to progress into the subsequent aligner upon a score of 1 for aligner pressure and the presence of air gaps. The PI ensured that every patient clearly understood their responsibilities and had received thorough instruction on how to progress through aligners. Patients were instructed not to progress into the next aligner without meeting the criteria.

The PI evaluated aligner pressure, fit and tracking consistent with usual and customary INVISALIGN™ clinical guidelines, after each test period.

Patients were made aware of the importance of wearing aligners 22 or more hours per day, removing them only for hygiene and meals. The PI checked that patients remained compliant with aligner wearing and ORTHOPULSE™ use at each orthodontic appointment.

Patients were advised to immediately contact their orthodontic clinic if they experienced any difficulties with their aligners or ORTHOPULSE™. Patients were also informed to immediately report any adverse events experienced at any point in time during their treatment to their orthodontic clinic.

Panoramic radiographs were taken for each patient no earlier than 6 months from the start of their orthodontic treatment, in order to assess the presence of EARR per safety of the apparatus.

Measurement Method

Assessment of Tooth Movement

As anterior teeth crowding is treated with a series of aligners, the arch perimeter increases. This increase indicates alignment of the anterior teeth and tooth movement, which can be expressed in millimeters per week. Arch perimeter measurements, according to the methodology of Howe et al. (1983), was used to determine the rate of tooth movement during the baseline and ORTHOPULSE™ periods.

Mandibular digital models were obtained at the beginning and end of each test period (X0, X1, Y0 and Y1) and imported into the Rhinoceros 3D modeling software (Version 5.0, Copyright D Robert McNeel & Associates), which has a measurement accuracy of 0.01 mm. 8 points were defined in the software, 2 at the distal edges of the canines, and 4 at the center of the edge of each incisor. The arch perimeter was determined by tracing a line from the buccolingual center of the distal surface of the canines around the dental arch and over the incisal edges of the anterior teeth, terminating at the corresponding distal surface of the opposite canine. The linear length of the curve was measured using Rhinoceros's dimensional analysis.

The change in perimeter length during each test period was determined by subtracting the perimeter measurement at the end of the test period from that of the start, dividing it by the number of days between X0 and X1 (or Y0 and Y1), then multiplying by 7 days per week. Sample calculation is set forth below:

$${V}_{X} = \frac{( {X}_{1 perimeter} - {X}_{0 perimeter} ) * 7 days / week}{( {X}_{1 date} - {X}_{0 date} )}$$

Where V is rate of tooth movement in millimeters per week (mm/wk)

Where X is the test first test period

Where X_{1 date}−X_{0 date} is expressed in days

Where Xₚₑᵣᵢₘₑₜₑᵣ is the arch perimeter length expressed in millimeters.

The same formula applies to test period Y

The rate of tooth movement in mm/wk is compared between test period X and Y in order to express the difference in tooth movement between baseline and ORTHOPULSE™.

Assessment of EARR:

The Planmeca Proline XC (Planmeca, Helsinki, Finland) digital imaging device was used to take both sets of panoramic radiographs. X-rays are designed for full-view patient positioning using a three laser positioning light system and a Graphic User Interface. Midsagittal, horizontal, and focal layer positioning beams situate the patient to ensure that the images are symmetric and undistorted in the left-right direction, as well as in the forward tilt of the patient's head. This ensures a high degree of consistency between the two sets of panoramic radiographs.

Initial and interim (no earlier than 5.5 months into treatment) panoramic radiographs were analyzed using a GNU Image Manipulation Program (Version 2.8.14, ©2014 The GIMP Team), with a measurement precision of 0.1 mm. The radiographic images were standardized both in resolution and magnification factor by measuring the widths of one maxillary central incisor.

Measurements were performed by the same examiner. Root and crown measurements were performed on maxillary central incisors, lateral and incisors as per the methods of Krieger et al., (2013). “Apical root resorption during orthodontic treatment with aligners. A retrospective radiometric study.” Head & Face Medicine 9:21 and Fritz et al., (2003) “Apical root resorption after lingual orthodontic therapy.” Journal of Orofacial Orthopedics 6(64):434-442, each incorporated by reference herein. A line was defined by the connection of the mesial and distal cementoenamel junction (CEJ). The crown length was measured as the distance between the incisal edge and the CEJ. The root length was measured as the distance from the root apex to the CEJ junction.

Outcome Variables

Effectiveness

Effectiveness was determined by comparing the amount of tooth movement in mm per week between the baseline and ORTHOPULSE™ periods during aligner orthodontic treatment in the mandibular arch, utilizing arch perimeter analysis.

Safety

Safety of the apparatus was determined by observing the severity of EARR as well as by freedom from any significant adverse events during the course of ORTHOPULSE™ treatment.

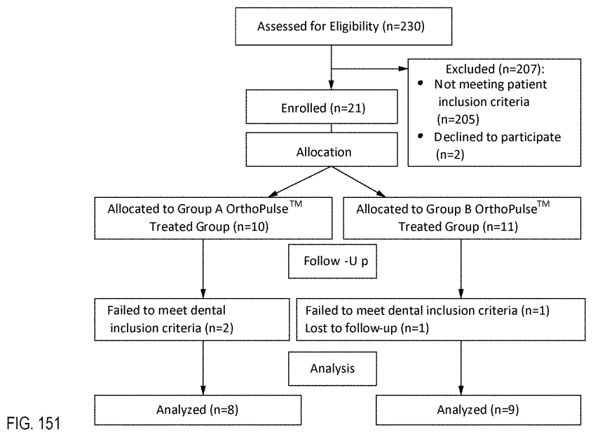

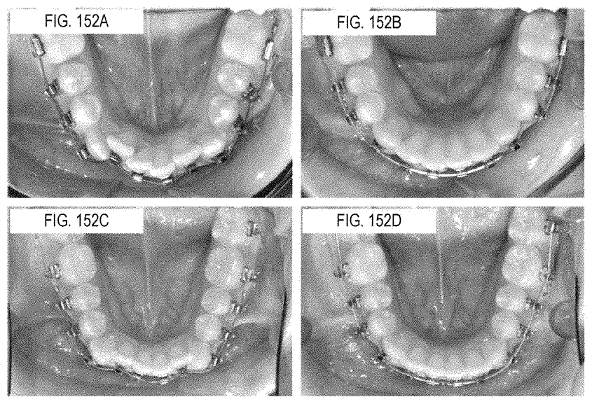

Table 2 below provides demographics statistics of the patients who were enrolled. 21 patients who were eligible for INVISALIGN™ treatment in a private practice office in Chandler, Ariz. were enrolled in the study. As shown in the consort chart at FIG. 151, 230 patients in total were screened for the study. Of them, 205 patients were not included, mainly because they did not meet the dental classification criteria. Additional reasons for non-inclusion were as follows:
PI concerned with future compliance
Patient concerned with compliance
Patient decided on braces rather than INVISALIGN™
Life schedule interfered with being able to make the more frequent appointments
Lived out of town/state and traveled to Phoenix/Chandler for appointments
Patient may be moving in near future or leaving for college

TABLE 2
Demographic characteristics of patients.

| | mean or % |
|---|---|
| N | 21 |
| Male | 8 |
| Female | 13 |
| Mean Age | 34.9 |
| StDev Age | 11 |
| Maximum Age | 53.1 |
| Minimum Age | 14.4 |
| Median Age | 35.6 |
| Caucasian | 71% |
| Hispanic | 14% |
| Indian | 5% |
| Black | 5% |
| Asian | 5% |

Ten (10) patients were enrolled in Group A, while 11 patients were enrolled in Group B. Of these 21 patients, 4 were removed proving a complete data set for 17 patients. See below for reasons for patient removal.

Raw perimeter measurement data for all patients is set forth in Table 3 below.

TABLE 3

| | | Baseline | Baseline | Baseline | OP | OP | OP | Reasons |
|---|---|---|---|---|---|---|---|---|
| | | Start | End | Time | Start | End | Time | for |
| Patient ID | Group | (mm) | (mm) | (days) | (mm) | (mm) | (days) | Removal |
| TD3- | B | 33.43 | 33.73 | 53 | 33.72 | 34.06 | 35 | |
| 001 | | | | | | | | |
| TD3- | A | 27.87 | 28.21 | 52 | 27.11 | 27.72 | 32 | |
| 002 | | | | | | | | |
| TD3- | B | 31.33 | 31.94 | 23 | 32.06 | 32.77 | 10 | |
| 003 | | | | | | | | |
| TD3- | A | 35.60 | 35.68 | 40 | 35.36 | 35.40 | 28 | |
| 004 | | | | | | | | |
| TD3- | B | 40.22 | 40.54 | 14 | 40.78 | 40.85 | 11 | |
| 005 | | | | | | | | |
| TD3- | B | 38.46 | 38.38 | 24 | 38.45 | 38.23 | 13 | |
| 006 | | | | | | | | |
| TD3- | B | 38.14 | 38.68 | 18 | 39.11 | 39.09 | 31 | |
| 007 | | | | | | | | |
| TD3- | A | 39.54 | 40.35 | 29 | 37.39 | 38.88 | 16 | |
| 008 | | | | | | | | |
| TD3- | A | 41.95 | 41.95 | 26 | 41.95 | 41.95 | 17 | Did not meet dental |
| 009 | | | | | | | | inclusion criteria. |
| | | | | | | | | Insufficient |
| | | | | | | | | initial crowding. |
| TD3- | B | 34.95 | 35.74 | 26 | 35.73 | 36.24 | 22 | |
| 010 | | | | | | | | |
| TD3- | B | 35.15 | 36.34 | 26 | 36.56 | 37.65 | 9 | |
| 011 | | | | | | | | |
| TD3- | A | 40.07 | 40.08 | 31 | 40.04 | 40.02 | 21 | Did not meet dental |
| 012 | | | | | | | | inclusion criteria. |
| | | | | | | | | Insufficient |
| | | | | | | | | initial crowding. |
| TD3- | A | 39.75 | 40.40 | 63 | 38.90 | 39.53 | 29 | |
| 013 | | | | | | | | |
| TD3- | B | 34.33 | 35.26 | 49 | | | | Lost to follow-up |
| 014 | | | | | | | | before completing OP. |
| TD3- | B | 32.66 | 32.84 | 32 | 32.87 | 33.14 | 12 | |
| 015 | | | | | | | | |
| TD3- | A | 34.54 | 34.59 | 24 | 34.25 | 34.46 | 19 | |
| 016 | | | | | | | | |
| TD3- | A | 37.45 | 37.72 | 16 | 36.32 | 37.10 | 15 | |
| 017 | | | | | | | | |
| TD3- | B | 39.93 | 39.78 | 33 | 39.63 | 39.41 | 14 | Did not meet dental |
| 018 | | | | | | | | inclusion criteria. Gaps. |
| TD3- | A | 35.03 | 35.22 | 16 | 34.90 | 35.11 | 12 | |
| 019 | | | | | | | | |
| TD3- | A | 39.26 | 39.87 | 37 | 37.48 | 38.94 | 33 | |
| 020 | | | | | | | | |
| TD3- | B | 34.04 | 35.32 | 30 | 35.67 | 36.36 | 24 | |
| 021 | | | | | | | | |

For the EARR analysis, tooth measurements at the start and no earlier than 6 months into treatment, were provided for 14 patients.

Three (3) patients were removed because they did not meet the initial dental inclusion criteria. Of these 3 patients, 2 demonstrated insufficient initial crowding, while one patient presented spaced between his anterior teeth. The fourth patient that was removed failed to continue attending her orthodontic appointments and was thus lost to follow-up.

No patient data was removed because of any major adverse event or side effects associated with the use of ORTHOPULSE™.

Panoramic radiographs at the start of treatment and no earlier than 6 months into treatment were provided for 14 patients. Interim radiographs were not provided for the 7 remaining patients since these patients had not yet reached the 6-month mark.

Adverse Events

Any unanticipated adverse device effects (UADE) were required to be reported to the Sponsor within 48 hours. A UADE is any serious adverse effect on health or safety or any life-threatening problem or death caused by, or associated with, a apparatus if that effect, problem, or death was not previously identified in nature, severity, or degree of incidence in the investigational plan or application (including a supplementary plan or application). A UADE also encompasses any other unanticipated serious problem associated with a apparatus that relates to the rights, safety, or welfare of subjects.

Events that are unfavorable to the patient and clinician are expected among patients receiving orthodontic treatment, particularly when related to oral pain. These events are minor and not considered to be important adverse events. They include:
Tooth or mouth discomfort
Jaw pain or discomfort
Mouth sores or canker sores
Minor abrasions to the oral mucosa

Tooth or mouth discomfort and jaw pain or discomfort was not considered important contributors to the outcome of this study, as they are commonly experienced during orthodontic treatment. However, the daily online questionnaires, which were intended only to guide patients with aligner progression at the appropriate time, also included a pain assessment question. Patients were required to rate, on a scale from 1 to 3, the level of pain they felt after placing their aligners. The written descriptions of each pain level were: 1—none or almost no pain, 2—somewhat painful, or 3—very painful. A rating of 3 was found rare among the pooled patients, with most patients never rating 3 at all.

As stated above, pain is expected during orthodontic treatment. Neither the PI nor the patients reported any pain beyond what was rated on the daily questionnaires, in spite of the questionnaires allotting room for comments. At no point in the duration of this study was it reported that anything beyond OTC medication used to alleviate pain.

Patients were informed to immediately contact their orthodontist if they experienced any type of apparatus difficulty. Patients were instructed to bring their apparatus to every orthodontic appointment, at which point each apparatus was thoroughly inspected for physical deformation and functionality. Nonetheless, there were no apparatus replacements or problems for any of the study patients during their ORTHOPULSE™ test period.

There were no reports of patients having any major negative experiences with the apparatus. No patients were discontinued from the study due to a negative adverse event. There were no negative adverse events or side effects reported, and none of the patients reported using anything beyond OTC medication to alleviate tooth and mouth discomfort. Furthermore, there was no gingival recession or tooth instability reported by the PI at any point during the course of the study.

Statistical Analysis and Results

Primary Data Analysis Results

Statistical testing was conducted using Stata software (version 12.1; StataCorp LP, College Station, Tex., USA). Seventeen (17) patients, who reached the primary outcome of the study, had a tooth movement rate of 0.126 and 0.231 mm/week for the baseline and ORTHOPULSE™ periods, respectively, as shown in Table 4.

Using analysis appropriate to crossover trials design ORTHOPULSE™ treatment effects were assessed while accounting for potential confounding carryover and period effects inherent in the crossover study design. Carryover effects refer to the potential carrying over of any residual effects for the treatment-first group (i.e. Group A patients that used ORTHOPULSE™ first) while period effects refer to potential learning as a result of accumulating experience from participating in the clinical trial (generally not a concern for a study such as this).

These effects were checked using values calculated from the perimeter rate changes of this study's two sequence groups (AB and BA). Two-sample t tests were used for normal data distributions, while the Wilcoxon rank sum test was used for non-normal distributions. Data normality was evaluated using a combination of visual (histogram and QQ-plots) and quantitative methods (Shapiro-Wilk test). Table 4 below provides a primer for the necessary calculations used in these Hills-Armitage checks. Carryover effects were assessed using a Wilcoxon rank sum test on the sum of the perimeter rate changes for both sequence groups (AB and BA). Period effects were evaluated using a Wilcoxon rank sum test on the difference of ORTHOPULSE™ and baseline perimeter change rates. Finally, treatment effects were tested using a two-sample t test on the difference of the first and second perimeter change rates.

TABLE 4
Hills-Armitage Prescribed Calculations for Testing

| | Sequence AB | | Sequence BA | |
|---|---|---|---|---|
| | Rate1 | Rate2 | Rate1 | Rate2 |
| Effect | Base- | ORTHOPULSE ™ | ORTHOPULSE ™ | Base- |
| | line | | | line |

| | | |
|---|---|---|
| Carryover | Rate1 + Rate2 | Rate1 + Rate2 |
| Period | Rate2 − Rate1 | Rate1 − Rate2 |
| Treatment | Rate1 − Rate2 | Rate1 − Rate2 |

Table 5 below shows the perimeter rate of tooth movement for study patients during their baseline period and ORTHOPULSE™ periods. The mean rate of tooth movement for baseline and ORTHOPULSE™ periods was 0.126 and 0.231 mm/week, respectively, with a p-value of 0.021, supporting a treatment effect. These results indicate that the rate of tooth movement for the ORTHOPULSE™ period was roughly 1.8-fold faster than that of the baseline period. Furthermore, the presence of period effects were, as expected, not supported. Carryover effects were also undetected.

TABLE 5
Perimeter rate of change

| | Perimeter Rate | | | | | Hills-Armitage | | |
|---|---|---|---|---|---|---|---|---|
| | Statistics | | | | | p-values | | |
| | Mean | | Min | Max | | Carryover | Period | Treatment |
| | Rate | | Rate | Rate | | Effect | Effect | Effect |
| | (mm/wk) | SD | (mm/wk) | (mm/wk) | N | P-value* | P-value* | P-value** |
| Baseline | 0.126 | 0.098 | 0.014 | 0.320 | 17 | | | |
| ORTHOPULSE ™ | 0.231 | 0.236 | 0.005 | 0.848 | 17 | 0.248 | 0.722 | 0.021 |

*Carryover effect and period effect p-values obtained using Wilcoxon Rank Sum Test (not normally distributed)
**Treatment Effect p-values obtained using Two-Sample T-Test (normally distributed)
p < 0.01 for significance

To assess intra-examiner reliability with respect to the perimeter measurements at X0, X1, Y0, and Y1, 5 patients were randomly selected and their arch perimeter measured at these four times points. The examiner was blind to patient identifiers throughout the process.

Measurements were collated and intraclass correlations (ICC) for two-way random-effects were calculated for each measurement group, in order to assess the accuracy and reproducibility of the examiner's measurements. Pearson's Interclass Correlation Coefficient (ICC) in combination with the Mann-Whitney U demonstrated an ICC over 0.99 when comparing the 5 re-measured models to the original measurements. This reflects strong measurement reliability, since values greater than 0.75 are typically regarded as excellent (Fleiss JL: The Design and Analysis of Clinical Experiments. New York, John Wiley Sons, 1986, pp 1-31).

Table 7 below shows the results of the EARR analysis, per the four maxillary incisors for 14 patients. The overall mean EARR is −0.673 mm, the negative value indicating root growth, rather than root loss. Therefore, there was no mean root resorption present after 6 months of ORTHOPULSE™ treatment. Furthermore, no patient experienced more than 1 mm of root resorption

TABLE 6
ITT perimeter rate of change

| | | | | | | Hills-Armitage | | |
|---|---|---|---|---|---|---|---|---|
| | Perimeter Rate | | | | | p-values | | |
| | Statistics | | | | | Carryover | Period | Treatment |
| | mean | | min | max | | Effect | Effect | Effect |
| | rate | SD | rate | rate | N | P-value* | P-value* | P-value** |
| Baseline | 0.110 | 0.097 | 0 | 0.320 | 21 | | | |
| ORTHOPULSE ™ | 0.198 | 0.223 | 0 | 0.848 | 21 | 0.291 | 0.379 | 0.035 |
| as ctrl | | | | | | | | |
| Baseline | 0.110 | 0.097 | 0 | 0.320 | 21 | | | |
| ORTHOPULSE ™ | 0.202 | 0.222 | 0 | 0.848 | 21 | 0.291 | 0.526 | 0.029 |
| as tx | | | | | | | | |

*Carryover effect and period effect p-values obtained using Wilcoxon Rank Sum Test (not normally distributed)
**Treatment Effect p-values obtained using Two-Sample T-Test (normally distributed)
p < 0.01 for significance

TABLE 7
Assessment of root resorption
Average amount of root resorption (mm)

| | |
|---|---|
| N | 14 |
| Mean | −0.673 |
| Std Dev | 0.756 |
| Median | −0.666 |
| Range | −2.249 |
| P-Value | 0 |

Positive sign indicates root resorption
2-sample t test used to test group differences
1-sample mean comparison test (root resorption = 0)

Panoramic radiographs of 4 patients were randomly chosen then measured. The examiner was blind to patient identifiers throughout the process. With respect to intra-examiner reliability, an ICC of 0.96 was obtained when comparing the 4 re-measured panoramic radiographs to the original measurements, reflecting strong measurement reliability and accuracy.

Intention-to-Treat (ITT) Analysis with Imputed Data

Regardless of dropouts, every data point during the time of original enrolment was included in the Intention-to-Treat (ITT) analysis for the purpose of evaluating apparatus safety and effectiveness. The ITT analysis was based on the initial treatment assignment and not on the treatment received or on whether the data is eligible. Therefore, any patient that dropped out of the study was accounted for in the ITT analysis by imputing the data. Because imputing data involves making assumptions about the outcomes in the missing data, it is not a true representation of the data. Therefore, ITT analysis is used as the secondary analysis in this study.

Two additional sets of data, which include the 4 previously removed patients, were analyzed as a robustness check. The 4 patients were removed due to not meeting the dental inclusion criteria (3 patients), and being lost to follow-up (1 patient). The patient lost to follow-up did not complete their ORTHOPULSE™ sessions and had her or his data imputed in two different ways (hence the two additional data sets).

In the first set, the last patient had her or his ORTHOPULSE™ data imputed as her or his baseline data (i.e. ORTHOPULSE™ data were replicated from baseline data). In the second set, the last patient had her or his ORTHOPULSE™ data imputed as the mean of the ORTHOPULSE™ group (i.e. her or his mean perimeter change rate was simply the mean rate of the ORTHOPULSE™ group). Table 6 above highlights the results from these two data sets.

Since all patients that were removed were still capable of provide baseline data, the results for these two analyses per baseline rates were the same, demonstrating tooth movement rate of 0.110 mm/week. When the missing ORTHOPULSE™ period is treated as control and treated as ORTHOPULSE™, the mean tooth movement rates show to be 0.198 (p-value=0.035) and 0.202 mm/week (p-value=0.029), respectively. In both types of analyses, the ORTHOPULSE™ period shows to be approximately 2-fold faster than that of the baseline period, with significance. The presence of period effects and carryover effects were, as expected, not supported.

The distinguishing feature of a crossover trial is that each patient serves as his or her own control. As a result, it eliminates biological differences and confounding variable between patients, thereby demonstrating the true effects of ORTHOPULSE™. Furthermore, this design is advantageous in terms of the power of the statistical test carried out to determine treatment effects: In comparison to parallel-group trials, crossover trials require lower sample sizes to meet the same criteria in terms of type I and type II error risks.

The study ran each patient through a baseline (no ORTHOPULSE™ treatment) period and a period of ORTHOPULSE™ treatment, with the order of these periods randomized. In doing so, the effect of intra-oral light therapy on a patient-by-patient basis was witnessed by noting individual decreases in treatment duration in combination with ORTHOPULSE™ treatment. The data were pooled, and results that better reflect individual biological variation were presented.

The baseline rate of tooth movement for all study patients was 0.126 mm/week, while their ORTHOPULSE period showed to be a rate of 0.231 mm/week. Not only does this show a 1.8-fold difference, but a p-value of 0.021 indicates that the treatment effects were significant. A carry-over of the effects of ORTHOPULSE™ into the baseline period was also analyzed. These findings show that there was no carry-over effect for Group A patients that had ORTHOPULSE™ treatment before their baseline. This suggests that the washout period was a sufficient duration of time, such that the effects of ORTHOPULSE™ did not influence baseline data. This finding also showed no significant difference of the ORTHOPULSE™ and baseline periods between the two groups, which suggests that there is no difference as to whether a patient carries out her or his ORTHOPULSE™ period before the baseline or vice versa.

Although patients progressed through aligners by rating specific criteria and were not permitted to progress unless reaching these criteria, progression through aligners was observed and monitored under the guidance of the PI. The PI evaluated aligner pressure, fit and tracking consistent with customary Align Technology, Inc. and INVISALIGN™ clinical guidelines, after each test period. In no case did patients present poor tracking, as reported by the PI.

The majority of root resorption associated with orthodontic treatment averages between 0.4 and 1.5 mm (Nimeri, G. et al., “The effect of photobiomodulation on root resorption during orthodontic treatment”, Clinical, Cosmetic and Investigational Dentistry 2014:6 1-8; Linge B O, Linge L. “Apical root resorption in upper anterior teeth”, Eur J Orthod. 1983; 5:173-183; Lund H et al., “Apical root resorption during orthodontic treatment. A prospective study using cone beam CT”, Angle Orthod. 2012; 82:480-487; Makedonas D et al., “Root resorption diagnosed with cone beam computed tomography after 6 months and at the end of orthodontic treatment with fixed appliances”, Angle Orthod. 2013; 83(3):389-393). The occurrence of root resorption is thought to be dependent on genetics, tooth root shape, mechanics applied and duration of treatment. Lateral and central incisors are the most affected teeth, and therefore the teeth that have been considered in the analysis. Data analysis of the study patients indicates no mean root resorption present after 6 months of ORTHOPULSE™ treatment. The results indicate a marginal amount of root elongation.

Perimeter measurement is useful and commonly used tool for quantifying dental crowding and determining the rate of tooth movement since it captures expansion of the arch, which is often the main goal of resolving malocclusion. However, it also has its shortcomings. First, it is important that the aligner staging for those aligners that are under consideration, aim to express individual tooth movement. As a result, patients whose treatment plan did not aim to express individual tooth movement through either of the baseline or ORTHOPULSE™ set of aligners were not considered.

In summary, this crossover study design, where patients serve as their own controls, shows that daily ORTHOPULSE™ treatments safely and effectively increase the rate of tooth movement during aligner orthodontic treatment

### Example 14

Typical, recommended switching rates for orthodontic aligners are about 20-22 hours per day. However, many patients find that wearing aligners for almost an entire day at a time is bothersome, interferes with speech, not aesthetically pleasing (e.g., in social situations), and/or disruptive to their work and/or other daytime activities.

Here it is shown that photobiomodulation using ORTHOPULSE™ and/or other accelerative apparatuses/techniques) (e.g., see FIG. 84) can be combined with aligner use to accelerate the rate of switching, while reducing aligner use. Provided herein is an example of patients using ORTHOPULSE™ together with aligners, instructing patients to wear their aligners about 8-12 hours per day, while replacing them about every 14 days. In some cases, the patients can switch aligners even more frequently (e.g., less than every 14 days) depending on the extent of desirable orthodontic tooth movement. Additionally, patients can use a daily questionnaire to determine an optimal switching rate for aligners regardless of the number of hours per day that they are worn.

A first patient, a 17 year-old female, had a Class III malocclusion and minor crowding in her lower arch, and had previously received orthodontic treatment in the form of conventional brackets and wires. She was instructed to wear INVISALIGN™ aligners for 12 hours per day, at night, and switched to new aligners weekly while performing ORTHOPULSE treatment on both arches daily using a wired ORTHOPULSE unit, having wired connectivity for charging and electronic communication. The predicted duration of treatment with aligners alone was about 12 months. The first patient completed her treatment with aligners and the ORTHOPULSE unit, using up all her aligners, in about 6 months.

A second patient, a 54 year-old female, had a Class II malocclusion and moderate crowding in both arches. She was instructed to wear INVISALIGN™ aligners 8 hours every day, at night, switched to new aligners weekly, and perform ORTHOPULSE treatment daily. The predicted duration of treatment with aligners alone was about 12 months. The second patient completed her treatment with aligners and the ORTHOPULSE unit, using up all her aligners, in about 6 months.

### Example 15—Intraoral Light Therapy-Induced Orthodontic Tooth Alignment Study

Subjects. Nineteen orthodontic subjects with Class I or Class II malocclusion and Little's Irregularity Index (LII)≥3 mm were selected from a pool of applicants, providing 28 total arches. Subjects (6 males, 13 females; each subject ranging from 11 to 18 years old) who presented for orthodontic treatment in a private practice office in Suwanee, Ga., were recruited for the study from a pool of applicants. The following inclusion criteria were used: presence of permanent dentition, eligible and scheduled for full mouth fixed orthodontic treatment, Class I or Class II malocclusion (no more than ½ cusp in Class II), nonextraction in all quadrants, non-smoker, good oral hygiene as determined by the investigator, and no adjunct treatment such as extra- or intraoral appliances. All subjects had a Little's Irregularity Index (LII) greater than or equal to 3 mm. Subjects were selected and treated between September 2011 and September 2013. None dropped out of the trial.

The first 8 subjects were enrolled as part of the control group, providing 10 arches eligible for the collection of alignment data (3 upper and 7 lower). The subsequent 11 patients, of whom 7 were treated on both arches, provided a total of 18 treatment arches (10 upper and 8 lower). Three lower arches were excluded from the intra-oral light therapy group for not meeting the initial dental criteria, as were 5 upper arches and 1 lower arch in the control group. The subjects of the intra-oral light therapy group were taught how to properly use the ORTHOPULSE™ device (e.g., such as the device illustrated in FIG. 57, or in FIGS. 65-66), and were instructed to report any adverse events immediately to the investigating orthodontist. Only 1 upper arch was excluded from the intra-oral light therapy group, as the patient did not maintain compliance for that arch. The subjects of the intra-oral light therapy group were instructed to use the ORTHOPULSE™ device for about 10 minutes per arch, totaling about 20 minutes per day.

Orthodontic alignment of anterior teeth. Subjects in the control group started orthodontic treatment before the intra-oral light therapy group. Due to a change in the type of brackets preferred by the clinical practice, the control group was bonded with 0.018-in slot self-ligating (SL) SPEED brackets (Hespeler Orthodontics, Cambridge, ON. Canada), but most subjects in the intra-oral light therapy group were bonded with 0.018-in slot conventionally-ligated (CL) Ormco Mini-Diamond twin brackets (Ormco, Glendora, Calif. USA). Both groups progressed through alignment with NiTi arch-wires from 0.014-in through to 0.018-in (Ormco), with arch-wires changed in the same manner. Complete records were obtained, including initial intraoral photographs, model casts and panoramic radiographs. Prior to bonding, the same operator (TS) collected all records. TO defined the date of bonding and the first ORTHOPULSE™ treatment, if applicable. Intraoral photos and ORTHOPULSE™ compliance were recorded at every follow-up appointment, scheduled every 3 weeks. When a subject's LII was visually estimated to have reached ≤1 mm, T1 was recorded and a T1 model was cast. A qualified technician evaluated T1 LII, and was blinded to which subjects the models originated from.

All anterior dentition crowding (LII) was measured to the nearest 0.1 mm with a fine-tip digital caliper (Point Digital Calliper SC02, Tresna Instruments, Guilin, China) by the same qualified technician. LII is the sum of the 5 linear distances from one anatomic contact point to the adjacent contact point of the 6 anterior teeth. It has been extensively used to document the degree of anterior tooth crowding, and others concluded that LII is an accurate and valid method for measuring anterior arch length discrepancy. LII measurements were made on initial models (T0) and aligned models (T1). The weekly rate of crowding resolution was calculated as:

[(T1 LII score)−(T0 LII score)]/[(T1 date)−(T0 date)]×7 days/week=rate per week

Device Description. Test subjects used an ORTHOPULSE™ device (Biolux Research, Vancouver, Canada), which produces near-infrared light with a continuous 850-nm peak wavelength. Subjects received an average of 3.8 min of buccal-only treatment per arch per day, using an average power density of 42 mW/cm² to achieve a mean energy density of approximately 9.3 J/cm² at the surface of the device's LED array. ORTHOPULSE™ includes an intraoral appliance connected to a handheld controller. The controller houses the microprocessor, LCD screen and controls for the menu-driven software. It connects to a medically approved wall wart UL-2601 certified power supply (FW7555m/15, UL rating 2601, Certified IEC 60601-1). The mouthpiece is made of a flex circuit of LED arrays embedded in medical grade silicone. Light is delivered through the buccal alveolar soft tissue into the alveolar mucosa. Any heat created as a by-product of light generation was monitored and kept below the thresholds of electromedical device safety standards. The device recorded every full intra-oral light therapy treatment session completed. This provided compliance data for each subject in the treatment group.

Statistical analysis. During study design, a sample size calculation was performed. Its parameters were based on conservative assumptions and the findings from a previous study (Kau et al. (2013). Prog Orthod. 14:30). A mean treatment effect of 2 times control alignment rate and a standard deviation of 50% of the mean were assumed. The power analysis used a two-tailed alpha of 0.05 and statistical power of 0.80—commonly accepted cut-offs. The analysis indicated that a minimum of 8 arches in each group was sufficient for the study to be clinically significant at the given effect size.

A variety of different tests were conducted to assess differences between treatment groups. Continuous variables (initial crowding and age) were evaluated for normalcy visually and via a Shapiro-Wilks test. Both variables were insufficiently normal, so the nonparametric Mann-Whitney U test was used to assess group differences. The chi-square test was used for categorical variables (sex, ethnicity, and arch). Although the difference in the number of arch type (upper vs. lower) provided in each group appears to be large, it was not found to be statistically significant (p=0.194). However, the group sizes are small, decreasing the accuracy of chi-square results. Because of this, arch as a possible predictor in a set of post-hoc Cox proportional hazards models was included.

After identifying any demographic and clinical differences, a log-rank test for equality of the survivor functions was used to evaluate any difference between the control and intra-oral light therapy groups. This test is appropriate to the nature of this study (two-samples with right skewed distributions measured in the form of time-to-event data).

Finally, the post-hoc Cox proportional hazards models were prepared to compare crowding resolution rate ratios for treatment type while controlling for demographic and clinical variables. To assess intra-examiner reliability of LII measurement, 8 models were randomly selected from the entire model pool to be re-measured three months before the last subjects completed alignment. Pearson's Interclass Correlation Coefficient (ICC) and a Mann-Whitney U were used to assess the accuracy and reproducibility of the LII method. Reliability analysis was completed following study completion.

All analyses were performed with the Stata 12 statistical package (StataCorp, College Station, Tex.).

Results. The mean alignment rate for the intra-oral light therapy group was significantly higher than that of the control group, with an LII change rate of 1.27 mm/week (SD 0.53, 95% CI±0.26) versus 0.44 mm/week (SD 0.20, 95% CI±0.12), respectively (p=0.0002). The treatment time to alignment was significantly smaller for the intra-oral light therapy group, which achieved alignment in 48 days (SD 39, 95% CI±39), while the control group took 104 days (SD 55, 95% CI±19, p=0.0053) on average. These results demonstrate that ORTHOPULSE™ use in combination with conventional brackets and wires increased the average rate of tooth movement by 2.9-fold, resulting in a 54% average decrease in alignment duration versus control. The average ORTHOPULSE™ compliance to daily treatments was 93% during alignment.

Results. The final sample consisted of 18 ORTHOPULSE™-treated arches and 10 control arches, all non-extraction with TO LII≥3.0 mm. All significance levels were assessed using a p-value cut-off of 0.05. There were no significant differences in age, sex or ethnicity between the two groups, nor in the ratio of mandible to maxillary arches and initial crowding.

Representative intraoral photographs of control (FIGS. 152A, 152B) and intra-oral light therapy (FIGS. 152C, 152D) treated patients are shown in FIGS. 152A-D. The treated group was found to have aligned at a rate of 1.27 mm/week compared to 0.44 mm/week for the control group (p=0.0002, Mann-Whitney U). The duration required for anterior tooth alignment was significantly less for the intra-oral light therapy group than for the control group (p=0.0053), with mean alignment times of 48 and 104 days, respectively. There was no statistically significant difference in initial LII between the two groups. However, the maximum starting LII was 8.80 mm for the control group and 14.58 mm for the intra-oral light therapy group. The Cox proportional hazard model has been used to examine difference in orthodontic tooth movement rates. These results are presented over five models. The first three models are a baseline set of controls covering all available demographic and clinical characteristics. Model 4 is the fully specified model, including intra-oral light therapy. To address any concerns surrounding potential model over-fitting, the three independent variables with the lowest p-values were removed from Model 4. The crowding resolution ratios were substantively unchanged by this (from 4.661 to 4.397, p<0.01). Therefore, these findings demonstrate that in the current dataset alignment rate is not influenced by any of these parameters, except for age; older patients appear to reach arch alignment later than younger patients. Initial crowding exhibited evidence of marginal significance, with higher LII associated with longer treatment (0.10>p>0.05 in models 4 and 5).

Only when ORTHOPULSE™ was used was a significant difference in alignment rate observed between groups. The difference in treatment time of the two groups shows a clear separation as early as 20 days, which was maintained throughout the duration of the study.

Based on these results, any null hypothesis that there would be no difference in the rate of anterior orthodontic alignment between the control and intra-oral light therapy groups is rejected.

Intra-examiner reliability was strong, with an ICC of over 0.96. Even when comparing only non-zero contact points, an ICC of over 0.94 was obtained. Values greater than 0.75 are typically regarded as excellent. A p-value of 0.87 was obtained using the Mann-Whitney U to test the median difference between the first and second measurement sets.

### Example 16

This Example demonstrates that Vitamin D supplementation improves the LED-induced photobiomodulation (“PBM”)-enhanced orthodontic tooth movement.

Materials and Methods

Animal Model: 131 healthy adult CRL-CD male rats with a body weight of 350-400 g were purchased from Charles River Laboratories and acclimatized for 3 days prior to the study at the Forsyth Institute. The protocol was approved by the Institutional Animal Care and Use Committee (IACUC) at the Forsyth Institute. In addition 6 animals, which were used for baseline, 125 animals were distributed into 5 groups with 25 in each. There are 5 treatment groups and one control group:

1) “TM”: conventional/orthodontic tooth movement (TM) via application of orthodontic force;

2) “TM+PBM”: photobiomodulation with LEDS+orthodontic tooth movement via application of orthodontic force;

3) “Vitamin D”: vitamin D only;

4) “TM+VitD”: Vitamin D supplied TM/orthodontic tooth movement via application of orthodontic force;

5) “TM+PBM+VitD” group: photobiomodulation with LEDS+orthodontic tooth movement via application of orthodontic force+Vitamin D; and

6) Control group: no application of TM or Vitamin D or PBM.

All groups had 5 time points (1, 3, 7, 21, and 28 days). All animals except for those of groups 3, 4 and 5 were fed rat chowder and water ad libitum. Vitamin D group received 25 microg/100 g of 1.25 (OH)₂ D₃ in their diets. Blood was sampled at baseline and all time points and analyzed for the vitamin D metabolites 25(OH)D₃ and 1,25(OH)₂D₃ using ELISA kits.

Orthodontic Tooth Movement: Animals that received orthodontic treatment underwent mesial tooth movement of the left maxillary 1st molar in a split mouth design. An orthodontic tooth movement model in the rat that has been established was used. In brief, the animals were anesthetized, a mini screw was inserted into palatal bone lingual to the maxillary incisors, and a coil spring was ligated to the left upper maxillary molar, activated for 10 mm and ligated around the mini screw implant and incisors. The miniscrew placement was a minimally invasive procedure, and a flap reflection was not necessary. The screws that were used were 1.5-diameter 4-mm long titanium mini-screws (Neuro MD screw, KLS Martin L. P., Jacksonville, Fla.) and were inserted through the mesial eyelet of the coil spring palatal to the incisors into the maxillary bone, avoiding the palatal bony suture. This was performed by manual manipulation. In humans, the process requires no special postoperative care; in this Example, no distress or any other symptoms in rats was observed. All animals of all groups, regardless of the miniscrew placement, received buprenorphine. Shallow notches were made around the left first molar crown and the incisor teeth using a small round bur in order to enhance the stability of the ligation. Flowable composite bonding material (Henry Schein Inc., Melville, N.Y., USA) was placed over the ligature wire on the mesio-buccal-distal aspects of the incisor teeth. This was done to prevent appliance loss, and protect the animal's lips from damage by the wire.

Photobiomodulation: For groups 2 and 5, a single dose of light was given. Application of light in the first session was accomplished under anesthesia and after shaving the hair on side of irradiation (left posterior maxilla). Post anesthesia application in following session was done using a rat-holding tube through a window, allowing unfettered access of the irradiation to maxilla achieved by shaving the right cheeks to be very thin. The animals were locally irradiated for 10 minutes in each session via an LED array in a housing placed on the surface of the shaved cheeks (total irradiation dose was 10 J/cm², at 850 nm). Irradiation was provided every day. At the day of euthanasia, ˜4 mL of blood sample was collected from each animal by cardiac venipuncture under anesthesia and serum was separated for analyses of calcium, PTH, and calcitonin. After the sacrifice, the maxillae and lower skull were defleshed, dissected and placed in formalin solution for storage.

Radiographic Analyses: In order to measure the amount of tooth movement in response to the applied force and the different radiation types and doses to determine the rate of tooth movement, radiographic images were taken on the dissected maxillae by using a Faxitron® imaging setup. Then, the radiographs were scanned and the mesiodistal distance between the most mesial aspects of the second to the most distal aspect of the first molar was measured in mm on a line parallel to a line following the midpalatal suture using the Olympus MicroSuite FIVE analysis software.

Then, in order to study the bone density and volumetric changes in the alveolar bone, micro-computerized tomography (micro-CT) images were obtained. All measurements were performed for the intrarticular area of the first molar (region of interest-1; ROI-1) and the area between the distal roots of the first and the mesial roots of the second molar (ROI-2). Coronally, the ROI was determined by the slice where the five roots of the first molar were observed as separated. The apical limit was determined at the last level where all five roots could be identified. Using this standardized volume, the bone-related parameters were described as the bone volume (BV, mm³), the ratio of bone volume to the total volume (BV/TV), bone mineral density (BMD, mg HA/cm³), and bone mineral content (BMC, mg HA), where HA is hydroxyapatite.

Histological Analyses: After imaging was completed, tissues were processed for histological analyses; the maxillae were decalcified, sectioned and stained using the TRAP staining. An osteoclast/pre-osteoclast count was collected in the PDL area of the mesial root of 1st molar in three different vertical levels (most apical, middle and most coronal). Hematoxylin and eosin (“H&E”) staining was used for histomorphometric appraisal of calcified osseous tissues surrounding the 5 roots of the upper first molars.

After the radiographic examinations, the samples were decalcified in 10% EDTA in distilled water for 14 days, embedded in paraffin and transversely sectioned (5 μm-thick) through the 1^{st}, 2^{nd} and 3^{rd} molar roots using a microtome. Slides at three different vertical levels of the roots (apical, middle and coronal) were chosen and stained with H&E. Similar to the micro-CT measurements, two areas were morphometrically analyzed. ROI-1 corresponded to the area between the 1^{st} molar roots and ROI-2 corresponded to the area between the distal roots of the 1^{st} and the mesial roots of the 2^{nd} molars. Histomorphometric analyses were used to determine the amount of trabecular bone and the ratio to the total interradicular area compared to baseline levels. Three different vertical levels were used to create a three-dimensional assessment. ROI-1 was defined as a pentagon-shaped grid, each angle formed by the center of the five first molar roots. This area was 1.17 mm². ROI-2 was defined by a square-shaped grid with angles at the center of the two distal roots of the first and two mesial roots of the second molar. Within these grids, the total amount of bone and PDL was recorded in square millimeters and calculated as percentage of the total measured area. All drawings were performed using the Olympus MicroSuite FIVE analysis software.

In order to study the catabolic activity in the alveolar bone, slides at three different levels were stained with tartrate-resistant acid phosphatase (TRAP). First, images of the first and third molars were captured at 100× magnification centered within the geometrical center of the molar roots. Then, the number of TRAP-stained osteoclasts and pre-osteoclasts were recorded within the captured interradicular area. Cell counts were performed on each of the three different levels per specimen and averaged. In order to study the distal penetration extent of the light application, all measurements were also performed at the control side. In parallel, bone formation was measured histologically by the osteocalcin using immunohistochemistry.

Statistical Analyses: Inter-group comparisons were made using ANOVA with Bonferroni's correction for multiple groups. Intra-group comparisons were made using t-test for repeated measurements.

Results

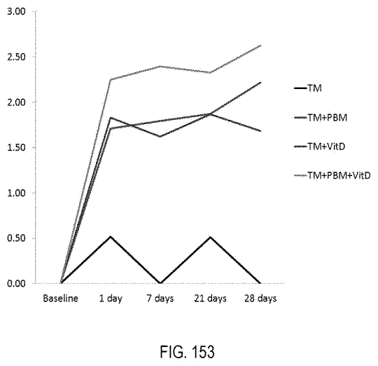

Administration of light increased the amount of conventional tooth movement by 2.8-fold over 28 days (FIG. 153, illustrating groups 1 (“TM”), 2 (“TM+PBM”), 4 (“TM+VitD”), and 5 (“TM+PBM+VitD”)). As illustrated in FIG. 153, Vitamin D and orthodontic tooth movement (group 4) enhanced the rate of tooth movement compared to conventional tooth movement alone (group 1) (p<0.05) at Day 1 and Day 21. A higher rate of tooth movement was observed with the combined administration of light and Vitamin D with conventional/orthodontic tooth movement (group 5) at all time-points compared to TM (group 1) and TM+VitD groups (group 4) (p<0.05), and at Day 1 and Day 3 compared to TM+PBM group (group 2).

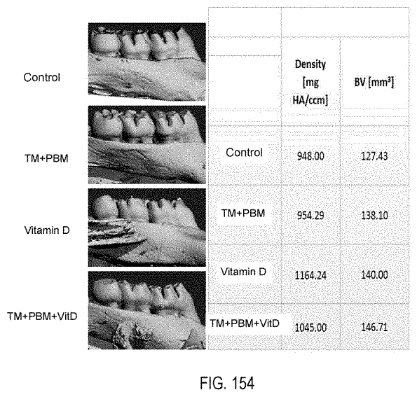

Vitamin D increased the density and volume of the bone as assessed by micro-CT with or without the administration of light (FIG. 154, illustrating groups 6 (“Control”), 2 (“TM+PBM”), 3 (“Vitamin D”), and 5 (“TM+PBM+VitD”).

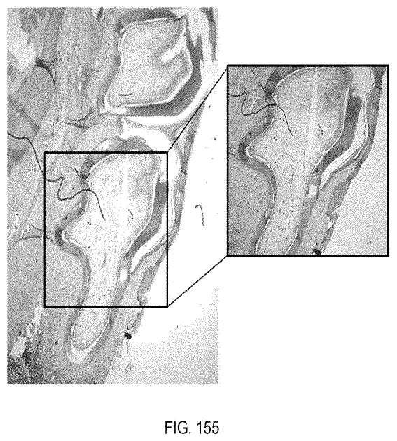

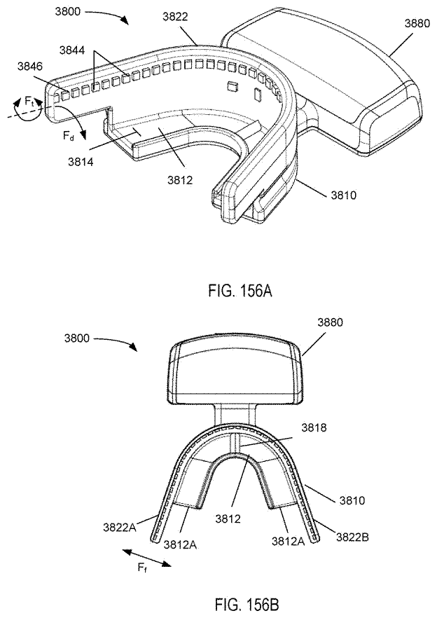

TRAP activity and Osteocalcin expression in group 3 at Days 3 and 21 were comparable to group 1, while the TM+PBM (group 2) and TM+PBM+VitD (group 5) groups saw a simultaneous activation of osteoclastic and osteoblastic activities up to 7 days followed by a reduced osteoclastic activity (FIG. 155, illustrating group 5).

Conclusion

These data show that administration of light accelerates tooth movement and stabilizes the anabolic activity of Vitamin D.

### Example 17

A patient is prescribed an orthodontic treatment program that includes the use of one or more removable orthodontic appliances. More particularly, the prescription prescribes that the patient wear the removable appliance, such as an aligner, for a predetermined number of hours (e.g., at least 22 hours) per day for at least a predetermined period of time (e.g., at least 1 week or at least 2 weeks). A compliance monitoring device is removably bonded to an outer surface of the patient's tooth. The compliance monitoring device includes a sensor that detects the presence of the orthodontic appliance when the appliance is at least partially disposed within the patient's mouth. When the orthodontic appliance is disposed at least partially within the patient's mouth, in a prescribed manner, the orthodontic appliance activates the sensor (e.g., a magnet coupled to an aligner activates a semiconductor of the compliance monitoring device). In this manner, the sensor detects the presence of the orthodontic appliance within a predetermined distance of the compliance device. One or more instances of activation of the sensor are stored as input data by the compliance device. The compliance device also stores a date and time associated with the instance of the activation.

The sensor detects the removal of the orthodontic appliance from being within the predetermined distance of the compliance device, e.g., due to a deactivation of the compliance device caused by the removal of the removable appliance. The compliance device stores one or more instances of deactivation of the sensor, such as may occur when the orthodontic appliance is removed from being disposed within the patient's mouth in the prescribed manner. The compliance device stores time and/or date data associated with each activation and subsequent deactivation of the sensor (or compliance device), thereby storing a log of activations/deactivations that occur over a period of time (e.g., about 24 hours, about 48 hours, about 72 hours, or the like). The compliance device can also store the amount of time the compliance device was within the patient's mouth and/or the amount of time the compliance device was absent from the patient's mouth, following the device's removal therefrom, based on an activation and/or deactivation of the sensor. The patient at least partially disposes a light therapy apparatus, as described herein, within the patient's mouth, for example, such that one or more light emitters embedded in a flange of the light therapy apparatus overlie the alveolar mucosa of the patient. The flange contacts the alveolar mucosa. The light therapy apparatus, when disposed within the patient's mouth, emits light to the alveolar mucosa.

During the light therapy, or within about 1 minute before or after the light therapy, the compliance device emits light via an LED to a photosensor of the light therapy apparatus. The light includes at least a portion of the stored data. The light therapy apparatus receives the optical/light signal including the stored data. The light therapy apparatus converts the light signal to electronic data representative of the stored data. The light therapy apparatus processes the data, including comparing the data to the prescribed treatment program, to determine patient compliance, and optionally transmits the data to an external computer for such data processing. More specifically, the data are processed to determine whether the orthodontic appliance was detected by the compliance device for at least the predetermined number of hours per day (or at multiple time intervals over the course of the day, thereby being representative of the predetermined number of hours per day) for at least the predetermined duration (e.g., one or two weeks). If so, the patient is determined to have complied with the treatment program, and more particularly with the prescribed removable appliance usage. If orthodontic appliance usage is detected, but the data are not indicative of total compliance, a percentage of compliance is determined. For example, a determination is made based on the data of a percentage of time for a particular date that the removable appliance was detected. Alternatively, a determination is made based on the data of a percentage of days over the total duration that the removably appliance was detected.

During, or within about 1 to 5 minutes before or after the light therapy, at least some of light emitted by the light therapy apparatus is received by the compliance device. The received light energy is converted within the compliance device to electricity that charges the battery of the compliance device.

### Example 18

A patient is prescribed an orthodontic treatment program that comprises self-administering light from one or more removable orthodontic appliances. More particularly, the program prescribes that the patient wear a removable appliance, such as an aligner, for a predetermined number of hours (e.g., at least 22 hours) per day for at least a predetermined period of time (e.g., at least 1 week or at least 2 weeks). A compliance monitoring device is removably bonded to an outer surface of the patient's tooth. The compliance device comprises a timer that is configured to track the amount of time that the orthodontic aligner is not disposed in the patient's mouth and a force sensor configured to detect the presence of an orthodontic aligner. The force sensor comprises a switch, optionally encapsulated in silicone, such that when the aligner is in place over the compliance device, the switch is activated by being depressed by the aligner. When depressed continuously (e.g., for more than 5-10 seconds), the force sensor triggers a command to turn off the timer. Thus, the compliance monitoring device is configured to detect the amount of time that an orthodontic appliance is not placed in a patient's mouth. The compliance device is configured to store time and/or date data associated with each activation and subsequent deactivation of the sensor, and can accordingly create or store a log of the number and duration of activations/deactivations that occur over a period of time (e.g., about 24 hours, about 48 hours, about 72 hours, or the like). The compliance device can also store the amount of time the compliance device was within the patient's mouth and/or the amount of time the compliance device was absent from the patient's mouth, following the device's removal therefrom, based on an activation and/or deactivation of the sensor.

Data from the compliance device is collected via a probe. The probe is a handheld appliance comprising a photodiode and an LED, as described herein. The probe is configured to be placed in close proximity to, or in direct contact with, the compliance monitoring device and to optically couple to the compliance monitoring device while the compliance monitoring device is within the patient's mouth. The probe tip, which houses the photodiode and the LED, is placed within the patient's mouth adjacent to the compliance monitoring device. The probe sends a light pulse to the compliance monitoring device to activate the data communication mode in the compliance monitoring device. Once the data communication mode is activated, the compliance monitoring device transmits data obtained by the sensor (and stored in memory) to the probe using pulsed light. The probe tip is removed from the patient's mouth, and the compliance monitoring device exits the data communication mode. The probe then communicates the data to a processor of a computer using a wired or wireless connection. The processor receives the raw data and optionally compares it to an orthodontic treatment program to determine whether the patient is in compliance with the treatment program. The computer displays information related to the user's compliance (e.g., whether the user was in overall compliance with the treatment program, and when and how long the user wore the one or more removable orthodontic appliances, e.g., aligners).

### CONCLUSION

While particular embodiments of the present invention have been shown and described herein, such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A compliance device that is configured to be disposed within a patient's mouth, the compliance device comprising a sensor configured to detect at a first time and at a second time an input associated with whether an orthodontic appliance is at least partially disposed within the patient's mouth, the second time being subsequent to the first time, the compliance device configured to be coupled to the orthodontic appliance or to a tooth of the patient, the compliance device configured to store data associated with input detected by the sensor at the first time and the second time, the compliance device comprising a transceiver or a light emitter.

2. The compliance device of claim 1, further comprising a casing configured to be bonded to the tooth.

3. The compliance device of claim 1, wherein the input is a detection of a capacitance change.

4. A system comprising:
the compliance device of claim 1, and;
a light therapy apparatus comprising a mouthpiece configured to be disposed within the patient's mouth proximate to or in contact with the compliance device, the light therapy apparatus comprising a transceiver or a light emitter, the transceiver and the light emitter being configured to receive a wireless signal from the compliance device, the signal comprising data associated with input detected by the sensor at the first time and the second time.

5. The system of claim 4, further comprising:
an orthodontic appliance configured to be at least partially disposed within the patient's mouth, the compliance device coupled to the orthodontic appliance.

6. The system of claim 4 or 5, wherein:
the signal is a first signal;
the input comprises an indication of a capacitance change, a pressure, magnetism, or a physiological parameter, and;
the light therapy apparatus is configured to wirelessly transmit a second signal to an external electronic device, the second signal comprising data associated with the input.

7. The system of claim 5, wherein the sensor is configured to detect a capacitance change produced by the orthodontic appliance when the orthodontic appliance is disposed within a predetermined distance from the compliance device.

8. The system of claim 5, wherein the compliance device includes a magnet configured to be bonded to the tooth, the sensor configured to detect magnetism produced by the appliance when the appliance is disposed within a predetermined distance of the magnet.

9. The system of any one of claims 5-8, wherein the orthodontic appliance is an aligner, and at least a portion of the aligner is optically transparent or optically translucent.

10. The system of any one of claim 5-9, wherein the orthodontic appliance is a removable aligner.

11. The system of any one of claims 5-10, wherein the orthodontic appliance is a first orthodontic appliance of a plurality of orthodontic appliances, the compliance device is a first compliance device, the system further comprising:
a second compliance device coupled to a second orthodontic appliance of the plurality of orthodontic appliances, wherein the second compliance device (i) does not comprise a sensor configured to detect the input or (ii) is configured to not detect the input at either the first time or the second time.

12. The system of claim 11, wherein:
the input is the physiological parameter, and;
the second compliance device (i) does not comprise a sensor configured to detect the physiological parameter and (ii) is configured to not detect the physiological parameter at either the first time or the second time.

13. The system of any one of claims 4-12, wherein the compliance device comprises a light emitter configured to optically communicate the signal to the light therapy apparatus.

14. The system of any one of claims 4-13, wherein:
the light emitter is a first light emitter;
the light therapy apparatus comprises a second light emitter that is different than the first light emitter, and;
the second light emitter is configured to optically receive, from the first light emitter, the signal from the compliance device.

15. The system of claim 14, wherein the light therapy apparatus is configured to provide power to the compliance device via light emitted by the second light emitter.

16. The system of claim 14 or 15, wherein the first emitter and the second emitter are light emitting diodes (LEDs).

17. The system of any one of claims 4-14, wherein the compliance device further comprises a rechargeable power source, and the compliance device is configured to use light energy received from the light therapy apparatus to charge the power source.

18. A compliance device that is configured to be disposed within a patient's mouth, the compliance device comprising a sensor, the sensor being configured to detect whether an orthodontic appliance is disposed within the patient's mouth, the compliance device comprising a power source that is configured to be recharged based on light received from a light therapy apparatus when disposed within the patient's mouth, the compliance device further comprising a transceiver that is configured to wirelessly communicate to the light therapy apparatus a signal that includes data associated with detection by the sensor.

19. The compliance device of claim 18, being configured to be coupled to the orthodontic appliance or to a tooth of the patient.

20. A system comprising the compliance device of claim 18 or 19 and a light therapy apparatus.

21. An orthodontic appliance that is configured to be disposed within a patient's mouth, the orthodontic appliance comprising a compliance device, the compliance device being configured to detect a temperature of the patient's mouth at a first time and a second time, the second time being subsequent the first time, the compliance device being configured to optically and wirelessly communicate to a light therapy apparatus when disposed within the patient's mouth a signal associated with the temperature of the patient's mouth at the first time and the temperature within the patient's mouth at the second time.

22. The orthodontic appliance of claim 21, wherein the compliance device is embedded in the orthodontic appliance.

23. A method, comprising:
allowing a light therapy apparatus to receive from a compliance device a signal, the light therapy apparatus comprising a mouthpiece and a plurality of light emitters, the mouthpiece and the plurality of light emitters being configured to be disposed within a patient's mouth, the compliance device being coupled to (i) a removable orthodontic appliance that is configured to be disposed within the patient's mouth or (ii) a tooth of the patient, the compliance device being configured to detect a temperature of the patient's mouth at a first time and a second time, the second time being subsequent the first time, the signal comprising data associated with the temperature of the patient's mouth at the first time and the second time.

24. The method of claim 23, wherein the signal is a first signal, and wherein the method further comprises allowing the light therapy apparatus to wirelessly transmit to an external electronic device a second signal, the second signal comprising data associated with the temperature of the patient's mouth at the first time and the second time.

25. The method of claim 24, wherein the orthodontic appliance is a first orthodontic appliance of a plurality of orthodontic appliances, wherein the compliance device is a first compliance device, wherein the first compliance device is coupled to the first orthodontic appliance and wherein the method further comprises:
allowing the light therapy apparatus to optically receive from a second compliance device an indication of additional temperature information, the second compliance device being coupled to a second orthodontic appliance, the second orthodontic appliance being of the plurality of orthodontic appliances, and;
generating compliance information based on the data from the first signal and second signal.

26. The method of claim 23, wherein:
the compliance device comprises a first light emitter that is configured to optically communicate the signal to the light therapy apparatus, and;
the light therapy apparatus comprises a second light emitter that is configured to optically receive tire signal.

27. The method of claim 26, wherein the compliance device comprises a rechargeable power source that is operably coupled to the first light emitter, wherein the method further comprises:
irradiating light to the first light emitter from that second light emitter such that the compliance device converts the light to energy capable of charging the rechargeable power source.

28. The method of any one of claims 23-27, wherein the removable orthodontic appliance is an optically transparent aligner.
